(19) United States
(12) Patent Application Publication
(10) Pub. No.: US 2011/0104713 A1
(43) Pub. Date: May 5, 2011

(54) PRODUCTS AND PROCESSES FOR MODULATING PEPTIDE-PEPTIDE BINDING DOMAIN INTERACTIONS

(76) Inventors: Michael B. YAFFE, West Roxbury, MA (US); Andrew E. H. Elia, Boston, MA (US); Peter Rellos, Herts (GB); Lewis C. Cantley, Cambridge, MA (US); Stephen J. Smerdon, London (GB); Isaac Manke, Cambridge, MA (US)

(21) Appl. No.: 12/893,957

(22) Filed: Sep. 29, 2010

Related U.S. Application Data
(63) Continuation of application No. 12/229,797, filed on Aug. 27, 2008, now abandoned.
Application No. 12/229,797 is a division of application No. 10/713,978, filed on Nov. 14, 2003, now abandoned.
(60) Provisional application No. 60/487,899, filed on Jul. 17, 2003.
(60) Provisional application No. 60/485,641, filed on Jul. 8, 2003.
(60) Provisional application No. 60/426,132, filed on Nov. 14, 2002.

(51) Int. Cl. G01N 33/53; C07K 1/00; G06G 7/48

(52) U.S. Cl. 435/7.8; 530/333; 703/11

(57) ABSTRACT

The present invention relates to therapeutic compounds and methods of use of these therapeutic compounds for treating cellular proliferative disorders. The invention also provides three-dimensional structures of a Polo-like kinase and methods for designing or selecting small molecule inhibitors using these structures, and the therapeutic use of such compounds. The invention also includes a method for identifying novel phosphopeptide-binding domains.

### CROSS-REFERENCE TO RELATED APPLICATIONS

This application is a continuation of U.S. patent application Ser. No. 12/229,797, filed on Aug. 27, 2008, which is a divisional application of U.S. patent application Ser. No. 10/713,978 filed on Nov. 14, 2003, which in turn claims the benefit of U.S. Provisional Patent Application Nos. 60/426,132, filed on Nov. 14, 2002, 60/485,641, filed on Jul. 8, 2003, and 60/487,899, filed on Jul. 17, 2003.

### STATEMENT AS TO FEDERALLY SPONSORED RESEARCH

The present research was supported by a grant from the National Institutes of Health-National Institute of General Medical Sciences (NIH-NIGMS; grant number GM52981). The U.S. government has certain rights to this invention.

### BACKGROUND OF THE INVENTION

The invention relates to compounds (e.g., peptidomimetics and non-peptides) that inhibit a cellular proliferative disorder and methods of treating such disorders. The invention also provides three-dimensional structures of a Polo-like kinase and methods for designing or selecting small molecule inhibitors using these structures. Desirably, these compounds have certain structural, physical, and spatial characteristics that enable the compounds to interact with specific amino acid residues.

Cyclin-dependent kinases (Cdks) have long been considered the master regulators of the cell-cycle, but an increasing number of diverse protein kinases are now emerging as critical components of cell-cycle progression. Among these are members of the Polo-like kinase family (Plks) that play key roles during all stages of mitosis and in the cell cycle checkpoint response to genotoxic stress. Many protein kinases involved in cell-cycle control function, in part, by generating phosphoserine/threonine-containing sequence motifs in their substrates that are subsequently recognized by phosphoserine/threonine-binding proteins. These include the WW and proline isomerase domain of Pin1 that regulates mitotic progression, 14-3-3 proteins that control the G2/M transition in response to DNA damage, and the WD40 repeat of Cdc4p which regulates S-phase entry.

In several instances, a phosphopeptide-binding domain and a kinase domain are combined within a single molecule, best exemplified by the SH2 domain-containing Src kinases and the Rad53p/Chk2-family of FHA domain-containing kinases. In these proteins the phosphopeptide-binding domain targets the kinase to pre-phosphorylated (primed) sites, mediates processive phosphorylation at multiple sites within a single substrate, or facilitates kinase activation. Polo-like kinases are distinguished by the presence of a conserved Ser/Thr kinase domain and a non-catalytic C-terminal region composed of two homologous ˜70-80 residue segments termed Polo-boxes.

Humans, mice and frogs each have three Plk homologues denoted Plk1, Plk2/Snk, and Plk3/Fnk/Prk, while budding yeast, fission yeast, and flies contain only a single Plk family member denoted Cdc5p, Plo1, and Polo, respectively. In addition, humans and mice have a serine/threonine kinase, Sak, that is an extremely divergent member of the Plk family, containing only a single Polo-box and lacking a canonical PBD.

The most extensively studied Polo-like kinases, Plk1 and Cdc5p, have been implicated in numerous mitotic processes including activation of Cdc25C and Cdc2-cyclinB at the G2-M transition, centrosome maturation and spindle assembly, cohesin release/cleavage during sister chromatid separation, anaphase promoting complex (APC) activation during mitotic exit, and septin regulation during cytokinesis. In contrast human Plk2 and Plk3 appear to serve different functions. Plk2 shows peak expression and activity in early G1, while Plk3 is activated by several stress response pathways, including DNA damage and spindle disruption. In fact, Plk3 plays some roles that may directly antagonize Plk1 function. For example, DNA damage directly inhibits Plk1, but activates Plk3 in an Ataxia-Telangiectasia-Mutated (ATM)-dependent manner. Consistent with these results, Plk1 overexpression causes oncogenic transformation in NIH 3T3 cells, while overexpression of Plk3 induces apoptosis.

### SUMMARY OF THE INVENTION

We have developed a proteomic approach for identifying targets downstream of kinases in signaling pathways. Our strategy involves using an immobilized library of partially degenerate phosphopeptides, biased toward a kinase phosphorylation motif, to isolate interacting effector proteins targeted by substrates of that kinase. Utilizing this approach for cyclin-dependent kinases, we discovered that the carboxy-terminal region of the cell cycle regulating kinase, Plk-1, encodes a phosphopeptide recognition domain that consists of the non-kinase region of this protein (amino acids 326-603). This phosphopeptide recognition domain, termed the Polo-box domain (PBD), binds phosphoserine and phosphothreonine residues in a sequence-specific context. Specifically, this PBD recognizes and binds to the core phosphopeptide sequence serine-phosphoserine or serine-phosphothreonine.

We performed oriented peptide library screening on the PBDs from all three human Plk homologues, as well as on the Plk1 orthologues Plx1 from Xenopus and Cdc5p from budding yeast. Despite differences in cellular function, we found that all PBDs show strong conserved selection for the core sequence S-[pSer/pThr]-P/X.

To determine the structural basis of PBD activity, the crystal structure of the human Plk1 PBD in complex with its optimal phosphothreonine-containing peptide was determined. We identified a mode of phosphopeptide binding that is unique among structurally characterized phosphodependent binding protein/modules and that is crucial for PBD targeting to substrates both in vitro and in vivo. The architecture of the Plk1 PBD differs significantly from that recently observed for homodimers of the single Polo-box from murine Sak, which lacks a formal PBD (Leung et al., Nat. Struct. Biol. 9:719-724, 2002). The Plk1 PBD represents a new protein fold. Site-directed mutagenesis based on the structural identification of critical phosphothreonine-binding residues has enabled us to demonstrate that phosphodependent substrate recognition by the PBD is necessary for proper mitotic progression. Furthermore, binding of the optimal Plk1 phosphopeptide to the PBD in full-length Plk1 enhances the in vitro activity of the kinase domain, leading to a model for Plk regulation in which intramolecular inhibition of the kinase by the PBD is relieved by PBD-ligand binding. We conclude that phosphoserine/threonine-dependent binding is a general feature of PBD activity across the Plk family and critically important for the function of this domain in Polo-like kinase targeting and regulation. These studies have identified sites that may be targeted in designing therapeutics useful in treating diseases or disorders characterized by inappropriate cell cycle regulation or inappropriate cell death.

We applied the same proteomic approach to identify phosphopeptide-binding modules mediating signal transduction events in the DNA damage response pathway. Using a library of partially degenerate phosphopeptides biased to resemble the phosphorylation motif of the phosphoinositide-like kinases ATM and ATR, we identified tandem BRCT domains in PTIP and BRCA 1 as phosphoserine (pSer)- or phosphothreonone (pThr)-specific binding modules that recognize a subset of ATM (ataxia telangiectasia—mutated) and ATR (ataxia telangiectasia—and RAD3-related)-phosphorylated substrates following γ-irradiation. PTIP tandem BRCT domains are responsible for phosphorylation-dependent protein localization into 53BP1- and phospho-H2AX (_-H2AX)—containing nuclear foci, a marker of DNA damage. These findings provide a new molecular rationale for BRCT domain function in the signaling response to DNA damage and may help to explain why the BRCA1 BRCT domain mutation Met1775 3 Arg, which fails to bind phosphopeptides, predisposes women to breast and ovarian cancer.

In one aspect, the invention generally features computer containing a processor in communication with a memory; the memory having stored therein (i) at least one atomic coordinate, or surrogates thereof, from Table 5 for each of the following residues: His-538, Lys-540, Trp-414, or Leu-491 of a Polo-box domain or atomic coordinates that have a root mean square deviation of the coordinates of less than 3 Å; and (ii) a program for generating a three-dimensional model of the coordinates. In one embodiment, the coordinate is for a heteroatom. In another embodiment, the coordinate is for a side-chain atom. In another embodiment, the coordinate is for a side-chain and a heteroatom.

In another aspect, the invention generally features a computer containing a processor in electrical communication with a memory; the memory having stored therein (i) atomic coordinates, or surrogates thereof, as shown in Table 5 for atoms of residues His-538, Lys-540, Trp-414, or Leu-491 of a Plk1 Polo-box domain or atomic coordinates that have a root mean square deviation from the coordinates of the residues of less than 1, 2, 3, 4, or 5 Å; and (ii) a program for displaying a three-dimensional model of the Polo-box domain.

In another aspect, the invention provides a computer containing a processor in communication with a memory; the memory having stored therein (i) x-ray diffraction data for at least one of the non-hydrogen atoms of residues His-538, Lys-540, Trp-414, or Leu-491 of a Polo-box domain or x-ray diffraction data for amino acids that have a root mean square deviation from the backbone atoms of the residues of less than 1, 2, 3, 4, or 5 Å; and (ii) a program for generating a three-dimensional model of the Polo-box domain.

In another aspect, the invention provides a computer containing a processor in communication with a memory; the memory having stored therein a pharmacophore model of a phosphopeptide that binds a Polo-box domain and a program for displaying the model, the model containing at least one of the following: a phosphate group on threonine that participates in at least 1 hydrogen-bonding interaction; and a serine at the pThr-1 position, where the Ser-1 side chain is directed towards the Plk1 surface. In one embodiment, the serine engages in at least two of the following (i) a hydrogen bonding interaction with Trp-414 main-chain atoms of PBD; (ii) a hydrogen bonding interaction with Leu-491 main-chain carbonyl of PBD; and (iii) a van der Waals interaction with CM from the Trp-414 indole side chain of PBD. In one embodiment, the model further comprises a Proline at the pThr+1 position, where the proline introduces a kink that allows a pThr+2 main chain amino group to contact PBD.

In another aspect, the invention provides a method of selecting or designing a candidate ligand for a Polo-box domain, the method involves the steps of: (a) generating a three-dimensional structure of a Polo-box domain having at least one atomic coordinate, or surrogate thereof, from Table 5 for each of the following residues: His-538, Lys-540, Trp-414, or Leu-491 or atomic coordinates that have a root mean square deviation from the coordinates of less than 1, 2, 3, 4, or 5 Å; and (b) selecting or designing a candidate ligand having sufficient surface complementary to the structure to bind a Polo-box domain in an aqueous solution. In another aspect, the invention provides a method for manufacturing a Polo-box domain ligand, the method involves the steps of: (a) obtaining the atomic coordinates of at least one residue of a Polo-box domain with a ligand; (b) determining one or more moieties in the ligand to be modified; where the modified ligand maintains the ability to bind the Polo-box domain; and (c) modifying the ligand based on the determination. In one embodiment, the method further involves crystallizing a Polo-box domain with a ligand. In another embodiment, the ligand specifically binds the Polo-box domain. In another embodiment, the modification increases the affinity of the ligand for the Polo-box domain. In another embodiment, the modification increases the solubility of the ligand. In another embodiment, the modification increases the half-life of the ligand in vivo.

In another aspect, the invention provides a method for manufacturing a Polo-box domain ligand, the method involves manufacturing a ligand that binds a Polo-box domain; where the ligand is designed or selected based on information obtained using a model of the atomic coordinates of at least a portion of the Polo-box domain.

In another aspect, the invention provides a method of evaluating the ability of a candidate ligand to bind a Polo-box domain, the method involves the steps of: (a) generating a three-dimensional structure of a Polo-box domain having at least one atomic coordinate, or surrogate thereof, from Table 5 for each of the following residues: His-538, Lys-540, Trp-414, or Leu-491 or atomic coordinates that have a root mean square deviation from the coordinates of less than 1, 2, 3, 4, or 5 Å; and (b) employing a means to measure the interaction between the candidate ligand and the Polo-box domain.

In another aspect, the invention provides a method of identifying a candidate ligand for a Polo-box domain, the method involves the steps of: (a) generating a three-dimensional pharmacophore model of Polo-box domain ligands using a computer of a previous aspect; and (b) selecting a candidate ligand satisfying the criteria of the pharmacophore model. In various embodiments, of any previous aspect, the method further involves determining the ability of the candidate ligand to bind the Polo-box domain in vitro or in vivo. In other embodiments, the method further involves determining the ability of the candidate ligand to alter the enzymatic activity of the Polo-box domain in vitro or in vivo. In other embodiments, the three-dimensional structure further comprises the hydrogen atoms of residues His-538, Lys-540, Trp-414, or Leu-491.

In various embodiments of the above aspects, the coordinate is for a heteroatom, or a side-chain atom, or a side-chain and a heteroatom. In other embodiments, the memory stores at least 1, 2, 3, 4, 5, 6, 7, 8, 9, or 10 coordinates or surrogates thereof for His-538; at least 1, 2, 3, 4, 5, 6, 7, 8, or 9 coordinates or surrogates thereof for Lys-540, at least 1, 2, 3, 4, 5, 6, 7, 8, 9, 10, 11, 12, 13, or 14 coordinates or surrogates thereof for Trp-414; or at least 1, 1, 2, 3, 4, 5, 6, 7, or 8 coordinates or surrogates thereof for Leu-491. In other embodiments, the coordinate is any one or all of the atomic coordinates in Table 5. In other embodiments of the previous aspect, the coordinates are for any residue required for the biological activity of a Polo box domain, or for binding a phosphopeptide or peptide mimetic. In other embodiments of any of the above aspects, root mean square deviation of the coordinates of less than 1, 2, 3, 4, 5, 6, or 7 Å.

In another aspect, the invention features a crystal of a Polo-like kinase complex containing a Polo-box domain bound to a phosphopeptide. In one embodiment, the Polo-like kinase is Plk-1 (SEQ ID NO: 1). In another embodiment, the Plk-1 comprises at least amino acids 1-603 of SEQ ID NO:1. In another embodiment, the Plk-1 comprises at least amino acids 95-603. In another embodiment, the Plk-1 comprises at least amino acids 326-603. In another embodiment, the Plk-1 comprises at least amino acids 367-603. In another embodiment, the phosphopeptide comprises the amino acid sequence [Pro/Phe]-[φ/Pro]-[φAla_{Cdc5p}/Gln_{Plk2}]-[Thr/Gln/His/Met]-Ser-[pThr/pSer]-[Pro/X] (SEQ ID NO: 2), where φ represents hydrophobic amino acids. In another embodiment, the phosphopeptide comprises the amino acid sequence MAGPMQ-S-pT-P-LNGAKK (SEQ ID NO: 3). In another embodiment, the Polo-like kinase is Plk-2 (SEQ ID NO: 4). In another embodiment, the Polo-like kinase is Plk-3 (SEQ ID NO: 5).

In another aspect, the invention provides a method of obtaining a structural model of a Polo-box domain of interest, the method involves homology modeling using at least a portion of the atomic coordinates in Table 5 and at least a portion of the amino acid sequence of the Polo-box domain of interest, thereby generating a model of the Polo-box domain of interest.

In another aspect, the invention provides a method of determining the three-dimensional structure of a Polo-box domain/phosphopeptide complex of interest, the method involves the steps of: (a) crystallizing the Polo-box domain/phosphopeptide complex of interest; (b) generating an X-ray diffraction pattern from the crystallized Polo-box domain of interest; and (c) applying at least a portion of the atomic coordinates in Table 5 to the diffraction pattern to generate a three-dimensional electron density map of at least a portion of the Polo-box domain/phosphopeptide complex of interest.

In another aspect, the invention features an isolated, less than full-length fragment of Polo-box domain containing residues 367-603 of human Plk-1 Polo-box domain) in complex with a phosphopeptide containing S-[pS/pT]-P/X, where X is any amino acid.

In another aspect, the invention features an isolated, less than full-length fragment of Polo-box domain containing residues residues 500-685 of human Plk-2 Polo-box domain in complex with a phosphopeptide containing S-[pS/pT]-P/X, where X is any amino acid.

In another aspect, the invention features an isolated, less than full-length fragment of Polo-box domain containing residues residues 421-607 of human Plk-3 Polo-box domain in complex with a phosphopeptide containing S-[pS/pT]-P/X, where X is any amino acid.

In another aspect, the invention features an isolated Polo-box domain protein or fragment thereof containing a mutation, where the mutation is (a) a mutation that enhances the ability of Polo-box domain to crystallize; (b) a mutation of a residue that is otherwise post-translationally modified in an organism used for recombinant expression; (c) a mutation of the NH₂— or COOH-terminal residue of Polo-box domain; (d) a mutation that increases or decreases the affinity of a Polo-box domain for a phosphopeptide; or (e) a mutation that alters the folding of Polo-box domain. In one embodiment, the PBD further comprises a mutation at His-538, Lys-540, Trp-414, or Leu-491. In other embodiments, the nucleic acid encodes a protein of any previous aspect.

In another aspect, the invention features a phosphopeptide containing the amino acid sequence [Pro/Phe]-[φ/Pro]-[φ/Ala_{Cdc5p}/Gln_{Plk2}]-[Thr/Gln/His/Met]-Ser-[pThr/pSer]-[Pro/X] (SEQ ID NO: 2), where φ represents hydrophobic amino acids. In one embodiment, the phosphopeptide comprises Pro-Met-Gln-Ser-pThr-Pro-Leu (SEQ ID NO: 6), where the phosphopeptide binds human Plk-1.

In another aspect, the invention features a phosphopeptide containing the amino acid sequence,

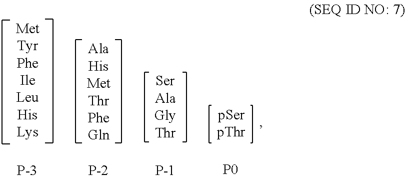

where pSer and pThr are phosphorylated serine and phosphorylated threonine, and where the amino acids designated in P-3, P-2, or P1 may be natural or unnatural amino acids. In one embodiment, the phosphopeptide of the previous aspect further contains the amino acid sequence,

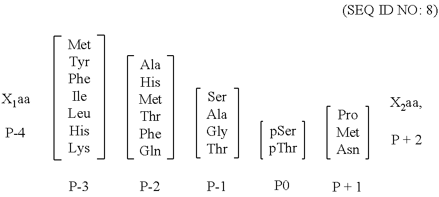

where X₁aa and X₂aa are any amino acids and where pSer and pThr are phosphorylated serine and phosphorylated threonine. In another embodiment, the X₁aa is proline and where X₂aa is any amino acid. In another embodiment, the X₁aa is any amino acid and where X₂aa is alanine, leucine, valine, isoleucine, phenylalanine, tyrosine, and tryptophan. In another embodiment, the X₂aa is leucine. In another embodiment, the amino acid at position P-3 is methionine. In another embodiment, the amino acid at position P-2 is glutamine. In another embodiment, the amino acid at position P-1 is serine. In another embodiment, the amino acid at position P0 is phosphorylated serine. In another embodiment, the amino acid at position P0 is phosphorylated threonine. In another embodiment, the amino acid at position P+1 is proline. In another embodiment, the amino acid sequence is Met-Gln-Ser-pThr-Pro-Leu or Met-Gln-Ser-pSer-Pro-Leu (SEQ ID NO: 9), where X₁aa is any amino acid and pThr is phosphorylated threonine and pSer is phosphorylated serine. In another embodiment, the phosphopeptide does not exceed 25 amino acids residues. In another embodiment, the phosphopeptide does not exceed 15 amino acids residues. In another embodiment, the phosphopeptide does not exceed 10 amino acids residues.

In another aspect, the invention features a pharmaceutical composition containing a therapeutic effective dose of any of the phosphopeptides of the previous aspects and a pharmaceutically acceptable excipient, where the pharmaceutical composition is useful for the treatment of a disorder characterized by inappropriate cell cycle regulation. In one embodiment, the cellular proliferative disorder is a neoplasm. In another embodiment, the composition further comprises a second chemotherapeutic agent. In another embodiment, the second chemotherapeutic agent is selected from the group consisting of paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, alemtuzumab, rituximab, streptozocin, tamoxifen, temozolomide, teniposide, 6-thioguanine, topotecan, trastuzumab, vincristine, vindesine, rofecoxib, celecoxib, etodolac and vinorelbine.

In another aspect, the invention features a method for treating or inhibiting a cellular proliferative disorder in a patient, the method involves administering a pharmaceutical composition of the phosphopeptide of a previous aspect, where the phosphopeptide is in an amount sufficient to treat or inhibit the cellular proliferative disorder in the patient. In one embodiment, method includes administering a second chemotherapeutic agent, the phosphopeptide and the chemotherapeutic agent are in amounts sufficient to treat or inhibit the cellular proliferative disorder in the patient, and where the chemotherapeutic agent is administered simultaneously or within 1, 2, 3, 5, 7, 10, 14, or 28 days of administering the phosphopeptide. In another embodiment, the second chemotherapeutic agent is selected from the group consisting of paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, alemtuzumab, rituximab, streptozocin, tamoxifen, temozolomide, teniposide, 6-thioguanine, topotecan, trastuzumab, vincristine, vindesine, rofecoxib, celecoxib, etodolac and vinorelbine, or any other chemotherapeutic known in the art. In other embodiments, the cellular proliferative disorder is a neoplasm.

In another aspect, the invention features a method for identifying a peptidomimetic compound that modulates Polo-like kinase biological activity, the method involves the steps of: a) contacting the phosphopeptide of a previous aspect and a Polo-box domain (PBD) polypeptide to form a complex between the phosphopeptide and the PBD; b) contacting the complex with a candidate compound; and c) measuring the displacement of the phosphopeptide from the PBD, where the displacement of the phosphopeptide from the PBD indicates that the candidate compound is a peptidomimetic compound that modulates Polo-like kinase biological activity.

In another aspect, the invention provides a method for identifying a peptidomimetic compound that modulates Polo-like kinase biological activity, the method involves the steps of: a) contacting the phosphopeptide of a previous aspect and a PBD in the presence of a candidate compound; and b) measuring binding of the phosphopeptide and the PBD, where a reduction in the amount of binding relative to the amount of binding of the phosphopeptide and the polypeptide in the absence of the candidate compound indicates that the candidate compound is a peptidomimetic compound that modulates Polo-like kinase biological activity. In one embodiment, the phosphopeptide or the PBD is detectably labeled. In another embodiment, the phosphopeptide and the PBD are differentially labeled. In another embodiment, the PBD is selected from a group consisting of the PBDs of CdcS, Plo-1, Polo, Plx-1, Plx-2, Plx-3, Plk-1, Prk/Fnk, Snk, and Cnk. In another embodiment, the PBD is Plk-1 PBD. In another embodiment, the Plk-1 PBD is human Plk-1 PBD.

In another aspect, the invention provides a method for identifying a binding pair consisting of a peptide and a peptide-binding domain, the method involes the steps of: a) providing a biased peptide library containing a collection of peptides fixed to a solid support, each peptide having at least two known amino acid residues whose position is invariant; b) providing a pooled cDNA library, where the cDNA library is positioned for protein expression; c) expressing the pooled cDNA library in the presence of a detectable label; d) contacting the peptide library and the expressed cDNA library; and e) detecting a peptide and peptide-binding domain interaction, where an interaction identifies a peptide and peptide-binding domain binding pair. In one embodiment, the biased peptide library is covalently bound to a solid support. In another embodiment, the biased peptide library is noncovalently bound to a solid support. In another embodiment, the peptide is a phosphopeptide and the peptide binding domain is a phosphopeptide binding domain.

In another aspect, the invention provides a method for identifying a binding pair containing a phosphopeptide and a phosphopeptide binding domain, the method involves the steps of: a) providing a biased phosphopeptide library, containing a collection of peptides fixed to a solid support, each peptide having at least two known amino acid residues whose position is invariant; where each phosphopeptide is covalently linked to a biotin group at the amino terminus; b) providing a pooled cDNA library, where the pooled cDNA library is positioned for protein expression; c) expressing the pooled cDNA library in the presence of a detectable label; d) contacting the phosphopeptide library and the expressed cDNA library; and e) detecting a phosphopeptide and the phosphopeptide binding domain interaction, where the presence of an interaction identifies a phosphopeptide and phosphopeptide binding domain. In one embodiment, method further comprises the steps of f) providing a non-phosphorylated peptide of step a), and g) detecting a peptide and phosphopeptide-binding domain interaction, where the absence of an interaction indicates the phosphopeptide and phosphopeptide binding domain interaction is authentic.

In another aspect, the invention provides a method for identifying a binding pair consisting of a peptide and a peptide-binding domain; the method involves the steps of: a) providing a biased peptide library containing a collection of peptides fixed to a solid support, each peptide having at least two known amino acid residues whose position is invariant; b) contacting the biased peptide library with a detectably labeled peptide library; and c) detecting a biased peptide and detectably labeled peptide interaction, where an interaction identifies a peptide and peptide-binding domain binding pair.

In another aspect, the invention features a method to identify phosphopeptide-binding modules, the method involves the steps of: (a) providing an immobilized phosphopeptide library and an immobilized peptide library; (b) contacting the libraries with a polypeptide or polypeptide fragment; and (c) detecting preferential binding, where preferential binding to the phosphopeptide library in comparison to the peptide library identifies the polypeptide or polypeptide fragment as a phosphopeptide binding module.

In another aspect, the invention provides a method to identify non-phosphopeptide-binding modules, the method involves the steps of: (a) providing an immobilized degenerate phosphopeptide library and an immobilized peptide library; (b) contacting the libraries with a polypeptide or polypeptide fragment; and (c) detecting preferential binding, where preferential binding to the peptide library in comparison to the phosphopeptide library identifies the polypeptide or polypeptide fragment as a non-phosphopeptide binding module.

In another aspect, the invention provides a method to identify phosphopeptide-binding modules in the DNA damage response pathway, the method involves the steps of: (a) providing an immobilized pSer or pThr degenerate phosphopeptide library and an immobilized Ser or Thr peptide library; (b) contacting the libraries with a polypeptide or polypeptide fragment; and (c) detecting differential binding, where preferential binding to the phosphopeptide library in comparison to the peptide library identifies the polypeptide or polypeptide fragment as a phosphopeptide binding module. In one embodiment, the phosphopeptide or peptide libraries do not have the amino acids Arg, Lys, or His in a degenerate position in the libraries. In another embodiment, the polypeptides or polypeptide fragments are in vitro translated (IVT) polypeptides.

In another aspect, the invention features a degenerate phosphopeptide containing a pSer or pThr that binds a BRCT domain. In one embodiment, the phosphopeptide further comprises an aromatic or aliphatic residue in the pSer or pThr +3 position; aromatic or aliphatic residues in the pSer or pThr +3 or +5 positions; a Gln or an aromatic or an aliphatic residue in the +1 position; or the amino acid sequence Y-D-I-(pSer or pThr)-Q-V-F—P—F (SEQ ID NO: 10).

In another aspect, the invention features a phosphopeptide binding module containing a BRCT tandem domain. In one embodiment, the BRCT tandem domain comprises at least 100 amino acids of the 3rd and 4th BRCT domains of PTIP. In another embodiment, the BRCT pair comprises at least 100 amino acids of the BRCT domains of BRCA 1. In another embodiment, the tandem domain functions as a single module in phosphopeptide binding.

In another aspect, the invention features an isolated fragment (e.g, 50, 100, 150, 200, 250, or 300 amino acids) of tandem BRCT domains of PTIP or BRCA1 in complex with a phosphopeptide containing a pSer or pThr amino acid.

In another aspect, the invention features a complex containing a tandem BRCT phosphopeptide binding module and a phosphopeptide containing a pSer or pThr. In one embodiment, the tandem BRCT phosphopeptide binding module is a fragment of PTIP in complex with a phosphopeptide. In another embodiment, the phosphopeptide further comprises an aromatic or aliphatic residue in the (pSer or pThr)+3 position; an aromatic or aliphatic residues in the (pSer or pThr)+3 or +5 positions a Gln, or an aromatic or aliphatic residue in the +1 position; or the amino acid sequence Y-D-I-(pSer or pThr)-Q-V-F—P—F (SEQ ID NO: 10). In another aspect, the invention provides a method for identifying a candidate compound for the treatment or prevention of a neoplasia, the method containing detecting binding of the phosphopeptide binding module to a phosphopeptide in the presence of the candidate compound, where a candidate compound that modulates the binding is a compound useful for the treatment or prevention of a neoplasia. In one embodiment, binding is detected using an immunological assay, an enzymatic assay, or a radioimmunoassay. In another embodiment, the phosphopeptide binding module or fragment thereof is an isolated phosphopeptide binding module. In another embodiment, the phosphopeptide binding module or fragment thereof is an isolated phosphopeptide containing a pSer or pThr. In one embodiment, phosphopeptide is fixed to a solid support. In another embodiment, the phosphopeptide binding module is a tandem BRCT binding domain. In another embodiment, the phosphopeptide binding module is fixed to a solid support. In another embodiment, the binding is assayed using an immunological assay, an enzymatic assay, or a radioimmunoassay. In another embodiment, the candidate compound is preincubated with the phosphopeptide binding module. In another embodiment, the candidate compound is preincubated with the phosphopeptide. In another embodiment, the phosphopeptide binding module and the phosphopeptide form a complex prior to being contacted with the candidate compound. In another embodiment, the candidate compound, the phosphopeptide and the phosphopeptide binding module are contacted concurrently.

In another aspect, the invention features a method for identifying a candidate compound useful in treating or preventing a neoplasia in a subject, the method involves: (a) providing a cell expressing a phosphopeptide binding module or fragment thereof and a phosphopeptide containing a pSer or pThr; (b) contacting the cell with a candidate compound; and (c) comparing binding of the phosphopeptide binding module and the phosphopeptide in the cell contacted with the candidate compound to the binding in a control cell, where a modulation of the binding identifies the candidate compound as a compound useful to treat or prevent a neoplasia in a subject. In one embodiment, phosphopeptide binding moduleand the phosphopeptide are expressed in a prokaryotic or a eukaryotic cell in vitro. In another embodiment, the phosphopeptide binding module is expressed endogenously by the cell. In another embodiment, the phosphopeptide binding module is expressed as a recombinant protein. In another embodiment, the cell is a neoplastic cell. In another embodiment, the neoplastic cell is a mammalian cell. In another embodiment, the neoplastic cell is a human cell. In another embodiment, the candidate compound decreases the affinity of the binding.

In another aspect, the invention features a pharmaceutical composition containing (i) a phosphopeptide containing a pSer or pThr and (ii) a pharmaceutically acceptable carrier, where the phosphopeptide is present in amounts that, when administered to a subject, ameliorates a neoplastic disease. In one embodiment, the compositions comprises a second chemotherapeutic agent. In another embodiment, the second chemotherapeutic agent is selected from the group consisting of paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, alemtuzumab, rituximab, streptozocin, tamoxifen, temozolomide, teniposide, 6-thioguanine, topotecan, trastuzumab, vincristine, vindesine, rofecoxib, celecoxib, etodolac and vinorelbine.

In another aspect, the invention provides a method for treating or inhibiting a cellular proliferative disorder in a patient, the method involves administering a pharmaceutical composition of the phosphopeptide of a previous aspect, where the phosphopeptide is in an amount sufficient to treat or inhibit the cellular proliferative disorder in the patient. In one embodiment, the method includes administering a second chemotherapeutic agent, the phosphopeptide and the chemotherapeutic agent are in amounts sufficient to treat or inhibit the cellular proliferative disorder in the patient, and where the chemotherapeutic agent is administered simultaneously or within fourteen days of administering the phosphopeptide. In another embodiment, the second chemotherapeutic agent is selected from the group consisting of paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, alemtuzumab, rituximab, streptozocin, tamoxifen, temozolomide, teniposide, 6-thioguanine, topotecan, trastuzumab, vincristine, vindesine, rofecoxib, celecoxib, etodolac and vinorelbine. In another embodiment, the cellular proliferative disorder is a neoplasm.

In another aspect, the invention features a method for identifying a peptidomimetic compound that modulates BRCT biological activity, the method involves the steps of a) contacting the phosphopeptide of claim a previous aspect and a BRCT binding domain domain polypeptide to form a complex between the phosphopeptide and the BRCT; b) contacting the complex with a candidate compound; and c) measuring the displacement of the phosphopeptide from the BRCT binding domain, where the displacement of the phosphopeptide from the BRCT binding domain indicates that the candidate compound is a peptidomimetic compound that modulates BRCT binding domain biological activity.

In another aspect, the invention features a method for identifying a peptidomimetic compound that modulates BRCT binding domain biological activity, the method involves the steps of: a) contacting the phosphopeptide of a previous aspect and a BRCT binding domain in the presence of a candidate compound; and b) measuring binding of the phosphopeptide and the BRCT binding domain, where a reduction in the amount of binding relative to the amount of binding of the phosphopeptide and the polypeptide in the absence of the candidate compound indicates that the candidate compound is a peptidomimetic compound that modulates BRCT binding domain biological activity. In one embodiment, the phosphopeptide or the BRCT binding domain is detectably labeled. In another embodiment, the phosphopeptide and the BRCT binding domain are differentially labeled. In other embodiments, the BRCT binding domain is BRCA 1 or PTIP. In another embodiment, the BRCT binding domain is of human BRCA1. In one embodiment, BRCT binding domain is of human PTIP.

In another aspect, the invention features a kit containing (i) a small molecule that binds a BRCT binding domain and (ii) instructions for administering the small molecule to a patient diagnosed with or having a propensity to develop a neoplasia. In one embodiment, the kit further comprises a second chemotherapeutic compound.

In another aspect, the invention features a method of assessing a patient as having, or having a propensity to develop, a neoplasia, the method involves determining the level of expression of an a BRCT binding domain nucleic acid molecule or polypeptide in a patient sample, where an increased level of expression relative to the level of expression in a control sample, indicates that the patient has or has a propensity to develop a neoplasia. In one embodiment, the patient sample is a blood or tissue sample. In another embodiment, the method comprises determining the level of expression of the BRCT binding domain nucleic acid molecule. In another embodiment, the method comprises determining the level of expression of the BRCT binding domain polypeptide. In another embodiment, the level of expression is determined in an immunological assay. In another embodiment, the method is used to diagnose a patient as having neoplasia.

In another aspect, the invention features a method to identify a peptide-binding module, the method involves the steps of: (a) providing an immobilized modified peptide library and an immobilized peptide library; (b) contacting the libraries with a polypeptide or polypeptide fragment; and (c) detecting preferential binding, where preferential binding to the modified peptide library in comparison to the peptide library identifies the polypeptide or polypeptide fragment as a modified peptide binding module.

In another aspect, the invention features a method for identifying a binding pair consisting of a modified peptide and a peptide-binding domain, the method involves the steps of: a) providing a biased peptide library containing a collection of modified peptides fixed to a solid support, each peptide having one amino acid residues whose position is invariant; b) providing a pooled cDNA library, where the cDNA library is positioned for protein expression; c) expressing the pooled cDNA library in the presence of a detectable label; d) contacting the peptide library and the expressed cDNA library; and e) detecting a modified peptide and peptide-binding domain interaction, where an interaction identifies a modified peptide and peptide-binding domain binding pair. In one embodiment, the amino acid contains a modification that is natural or unnatural. In another embodiment, the modification is selected from the group consisting of methylation, acetylation, ubiquitination, glycosylation, sumolation, or arsenylation, or any other modification known to the skilled artisan.

In various embodiments of any of the above aspects, the peptide includes unnatural amino acids as described herein.

By “analog” is meant a molecule that is not identical but has analogous features. For example, a peptide analog retains the biological activity of a corresponding naturally-occurring peptide, while having certain biochemical modifications that enhance the analogs function relative to a naturally occurring peptide. Such biochemical modifications might increase the analogs protease resistance, membrane permeability, or half-life, without altering, for example, ligand binding. An analog can include a non-natural amino acid.

In another example, a nucleic acid analog retains the ability to hybridize to a naturally-occurring corresponding nucleic acid sequence, while having certain biochemical modifications that enhance the analogs function relative to a naturally-occurring nucleic acid. In some nucleic acid analogs the sugar and/or the internucleoside linkage, i.e., the backbone, of the nucleotide units are replaced with novel groups. The base units are maintained for hybridization with an appropriate nucleic acid target compound. Peptide and nucleic acid modifications may be achieved by any of the techniques known in the art for derivatization of peptides or nucleic acids into fragments, analogs, or derivatives thereof. Such terms and in particular, “analog”, also specifically include peptide, non-peptide, peptide/nucleic acid hybrid molecules, small molecules and other compounds that function as Polo-like kinase nucleic acid or peptide mimics.

By “apoptosis” is meant the process of cell death where a dying cell displays at least one of a set of well-characterized biological hallmarks, including cell membrane blebbing, cell soma shrinkage, chromatin condensation, or DNA laddering.

By “biased phosphopeptide library” is meant a phosphoserine, phosphothreonine, and/or phosphotyrosine degenerate peptide library, wherein specific amino acid residues of the phosphopeptide are fixed so as to be expressed in all phosphopeptides in the specific library. For instance, a biased phosphopeptide library can be synthesized to contain the core sequence Ser-pSer-Pro or Ser-pThr-Pro. In a desirable embodiment, the amino acid residue adjacent to the phosphoserine, phosphothreonine, or phosphotyrosine residue is fixed.

By an “amino acid fragment” is meant an amino acid residue that has been incorporated into a peptide chain via its alpha carboxyl, its alpha nitrogen, or both. A terminal amino acid is any natural or unnatural amino acid residue at the amino-terminus or the carboxy-terminus. An internal amino acid is any natural or unnatural amino acid residue that is not a terminal amino acid.

As used herein, the terms “alkyl” and the prefix “alk-” are inclusive of both straight chain and branched chain groups and of cyclic groups, i.e., cycloalkyl and cycloalkenyl groups. Cyclic groups can be monocyclic or polycyclic and preferably have from 3 to 8 ring carbon atoms, inclusive. Exemplary cyclic groups include cyclopropyl, cyclopentyl, cyclohexyl, and adamantyl groups.

By “aromatic residue” is meant an aromatic group having a ring system with conjugated π electrons (e.g., phenyl or imidazole). The ring of the aryl group is preferably 5 to 6 atoms. The aromatic ring may be exclusively composed of carbon atoms or may be composed of a mixture of carbon atoms and heteroatoms. Preferred heteroatoms include nitrogen, oxygen, sulfur, and phosphorous. Aryl groups may optionally include monocyclic, bicyclic, or tricyclic rings, where each ring has preferably five or six members. The aryl group may be substituted or unsubstituted. Exemplary substituents include alkyl, hydroxyl, alkoxy, aryloxy, sulfhydryl, alkylthio, arylthio, halo, fluoroalkyl, carboxyl, carboxyalkyl, amino, aminoalkyl, monosubstituted amino, disubstituted amino, and quaternary amino groups.

By “aryl” is meant a carbocyclic aromatic ring or ring system. Unless otherwise specified, aryl groups are from 6 to 18 carbons. Examples of aryl groups include phenyl, naphthyl, biphenyl, fluorenyl, and indenyl groups.

By “heteroaryl” is meant an aromatic ring or ring system that contains at least one ring hetero-atom (e.g., O, S, N). Unless otherwise specified, heteroaryl groups are from 1 to 9 carbons. Heteroaryl groups include furanyl, thienyl, pyrrolyl, imidazolyl, pyrazolyl, oxazolyl, isoxazolyl, thiazolyl, isothiazolyl, triazolyl, oxadiazolyl, oxatriazolyl, pyridyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl groups.

By “heterocycle” is meant a non-aromatic ring or ring system that contains at least one ring heteroatom (e.g., O, S, N). Unless otherwise specified, heterocyclic groups are from 1 to 9 carbons. Heterocyclic groups include, for example, dihydropyrrolyl, tetrahydropyrrolyl, piperazinyl, pyranyl, dihydropyranyl, tetrahydropyranyl, tetrahydrofuranyl, dihydrothiophene, tetrahydrothiophene, and morpholinyl groups.

By “halide” or “halogen” or “halo” is meant bromine, chlorine, iodine, or fluorine.

The aryl, heteroaryl, and heterocyclyl groups may be unsubstituted or substituted by one or more substituents selected from the group consisting of C₁₋₅ alkyl, hydroxy, halo, nitro, C₁₋₅ alkoxy, C₁₋₅ alkylthio, trihalomethyl, C₁₋₅ acyl, arylcarbonyl, heteroarylcarbonyl, nitrile, C₁₋₅ alkoxycarbonyl, oxo, arylalkyl (wherein the alkyl group has from 1 to 5 carbon atoms) and heteroarylalkyl (wherein the alkyl group has from 1 to 5 carbon atoms).

By “biased phosphopeptide library” is meant a phosphoserine, phosphothreonine, and/or phosphotyrosine degenerate peptide library, wherein specific amino acid residues of the phosphopeptide are fixed so as to be expressed in all phosphopeptides in the specific library. For instance, a biased phosphopeptide library can be synthesized to contain the core sequence Ser-pSer-Pro or Ser-pThr-Pro. In a desirable embodiment, the amino acid residue adjacent to the phosphoserine, phosphothreonine, or phosphotyrosine residue is fixed.

By an “amino acid fragment” is meant an amino acid residue that has been incorporated into a peptide chain via its alpha carboxyl, its alpha nitrogen, or both.

A terminal amino acid is any natural or unnatural amino acid residue at the amino-terminus or the carboxy-terminus. An internal amino acid is any natural or unnatural amino acid residue that is not a terminal amino acid.

As used herein, the terms “alkyl” and the prefix “alk-” are inclusive of both straight chain and branched chain groups and of cyclic groups, i.e., cycloalkyl and cycloalkenyl groups. Cyclic groups can be monocyclic or polycyclic and preferably have from 3 to 8 ring carbon atoms, inclusive. Exemplary cyclic groups include cyclopropyl, cyclopentyl, cyclohexyl, and adamantyl groups.

By “aromatic residue” is meant an aromatic group having a ring system with conjugated π electrons (e.g., phenyl or imidazole). The ring of the aryl group is preferably 5 to 6 atoms. The aromatic ring may be exclusively composed of carbon atoms or may be composed of a mixture of carbon atoms and heteroatoms. Preferred heteroatoms include nitrogen, oxygen, sulfur, and phosphorous. Aryl groups may optionally include monocyclic, bicyclic, or tricyclic rings, where each ring has preferably five or six members. The aryl group may be substituted or unsubstituted. Exemplary substituents include alkyl, hydroxyl, alkoxy, aryloxy, sulfhydryl, alkylthio, arylthio, halo, fluoroalkyl, carboxyl, carboxyalkyl, amino, aminoalkyl, monosubstituted amino, disubstituted amino, and quaternary amino groups.

By “aryl” is meant a carbocyclic aromatic ring or ring system. Unless otherwise specified, aryl groups are from 6 to 18 carbons. Examples of aryl groups include phenyl, naphthyl, biphenyl, fluorenyl, and indenyl groups.

By “BRCA1 nucleic acid” is meant a nucleic acid, or analog thereof, that encodes BRCA1 or is substantially identical to Gene Bank Accession No: 30039658 (SEQ ID NO: 11).

By “BRCA 1 polypeptide” is meant a polypeptide, or analog thereof, substantially identical to BRCA1 Genbank Accession NO. 30039659 (SEQ ID NO: 12) and having BRCA1 biological activity.

By “BRCA1 biological activity” is meant function in a DNA damage response pathway or phosphopeptide binding.

By “BRCT nucleic acid is meant a nucleic acid, or nucleic acid analog, that encodes tandem BRCT domains. For example, a nucleic acid substantially identical to PTIP BC033781[21707457] (SEQ ID NO: 13), or NM_{—}007349 (PAX transcription activation domain interacting protein 1 mRNA) (SEQ ID NO: 14) or Gene Bank Accession No: AY273801[30039658] (SEQ ID NO: 11).

By “tandem BRCT polypeptide is meant a protein having at least 2 tandem BRCT domains. For example, a protein substantially identical to AAH33781 (SEQ ID NO: 15), NP_{—}031375 (SEQ ID NO: 16), or Genbank Accession NO. 30039659 (SEQ ID NO: 12).

By “candidate compound” is meant any nucleic acid molecule, polypeptide, or other small molecule, that is assayed for its ability to alter gene or protein expression levels, or the biological activity of a gene or protein by employing one of the assay methods described herein. Candidate compounds include, for example, peptides, polypeptides, synthesized organic molecules, naturally occurring organic molecules, nucleic acid molecules, and components thereof.

By “detectably-labeled” is meant any means for marking and identifying the presence of a molecule, e.g., a PBD-interacting phosphopeptide, a PBD, a nucleic acid encoding the same, or a peptidomimetic small molecule. Methods for detectably-labeling a molecule are well known in the art and include, without limitation, radionuclides (e.g., with an isotope such as ³²P, ³³P, ¹²⁵I, or ³⁵S) and nonradioactive labeling (e.g., chemiluminescent labeling or fluorescein labeling).

If required, molecules can be differentially labeled using markers that can distinguish the presence of multiply distinct molecules. For example, a PBD domain-interacting phosphopeptide can be labeled with fluorescein and a PBD domain polypeptide can be labeled with Texas Red. The presence of the phosphopeptide can be monitored simultaneously with the presence of the PBD.

By “diseases or disorder characterized by inappropriate cell cycle control” is meant any pathological condition in which there is an abnormal increase or decrease in cell proliferation. Exemplary diseases or disorder characterized by inappropriate cell cycle control include cancer or neoplasms, inflammatory diseases, or hyperplasias (e.g. some forms of hypertension, prostatic hyperplasia).

By “disease or disorder characterized by inappropriate cell death” is meant any pathological condition in which there is an abnormal increase in apoptosis. Exemplary diseases or disorders characterized by inappropriate cell death include neurodegenerative diseases (e.g., Alzheimer's, Huntington's, and Parkinson's disease), cardiac disorders (e.g., congestive heart failure and myocardial infarction), diabetic retinopathy, and age-related macular degeneration.

By “fragment” is meant a portion of a protein (50, 100, 150, 175, 200, 300, or 400 amino acids) or nucleic acid (50, 100, 150, 175, 200, 300, or 400 nucleic acids) that is substantially identical to a reference protein or nucleic acid, and retains at least 50% or 75%, more preferably 80%, 90%, or 95%, or even 99% of the biological activity of the reference protein or nucleic acid using a molting assay as described herein.

By “heteroaryl” is meant an aromatic ring or ring system that contains at least one ring hetero-atom (e.g., O, S, N). Unless otherwise specified, heteroaryl groups are from 1 to 9 carbons. Heteroaryl groups include furanyl, thienyl, pyrrolyl, imidazolyl, pyrazolyl, oxazolyl, isoxazolyl, thiazolyl, isothiazolyl, triazolyl, oxadiazolyl, oxatriazolyl, pyridyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl groups.

By “heterocycle” is meant a non-aromatic ring or ring system that contains at least one ring heteroatom (e.g., O, S, N). Unless otherwise specified, heterocyclic groups are from 1 to 9 carbons. Heterocyclic groups include, for example, dihydropyrrolyl, tetrahydropyrrolyl, piperazinyl, pyranyl, dihydropyranyl, tetrahydropyranyl, tetrahydrofuranyl, dihydrothiophene, tetrahydrothiophene, and morpholinyl groups.

By “halide” or “halogen” or “halo” is meant bromine, chlorine, iodine, or fluorine.

The aryl, heteroaryl, and heterocyclyl groups may be unsubstituted or substituted by one or more substituents selected from the group consisting of C₁₋₅ alkyl, hydroxy, halo, nitro, C₁₋₅ alkoxy, C₁₋₅ alkylthio, trihalomethyl, C₁₋₅ acyl, arylcarbonyl, heteroarylcarbonyl, nitrile, C₁₋₅ alkoxycarbonyl, oxo, arylalkyl (wherein the alkyl group has from 1 to 5 carbon atoms) and heteroarylalkyl (wherein the alkyl group has from 1 to 5 carbon atoms).

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “isolated polypeptide” is meant a polypeptide of the invention that has been separated from components which naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “modulate” is meant a change, such as a decrease or increase. Desirably, the change is either an increase or a decrease of at least 10%, 20%, 30%, 40%, 50%, 60%, 70%, 80%, 90% or 95% in expression or biological activity, relative to a reference or to control expression or activity, for example the expression or biological activity of a naturally occurring Polo-like kinase.

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (e.g., acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, lymphoma Hodgkin's disease, Waldenstrom's macroglobulinemia, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenriglioma, schwannoma, meningioma, melanoma, neuroblastoma, or retinoplastoma).

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

Specific examples of some preferred nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen et al. Science 199: 254, 1997). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)—CH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group.

Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines.

By “Pax2 trans-activation domain-interacting protein (PTIP) nucleic acid” is meant a nucleic acid, or analog thereof, substantially identical to Genebank Accession No:21707457 (SEQ ID NO: 13) or NM_{—}007349 (SEQ ID NO: 14).

By “Pax2 trans-activation domain-interacting protein (PTIP)” is meant a polypeptide, or analog thereof, substantially identical to Genebank Accession No: AAH33781.1 (SEQ ID NO: 15) or NP_{—}031375 (SEQ ID NO: 16), and having PTIP biological activity.

By “PTIP biological activity” is meant function in a DNA damage response pathway or phosphopeptide binding.

By “pharmaceutically acceptable excipient” is meant a carrier that is physiologically acceptable to the subject to which it is administered and that preserves the therapeutic properties of the compound with which it is administered. One exemplary pharmaceutically acceptable excipient is physiological saline. Other physiologically acceptable excipients and their formulations are known to one skilled in the art and described, for example, in “Remington: The Science and Practice of Pharmacy” (20th ed., ed. A. R. Gennaro A R., 2000, Lippincott Williams & Wilkins).

By a “peptidomimetic” is meant a compound that is capable of mimicking or antagonizing the biological actions of a natural parent peptide. A peptidomimetic may include non-peptidic structural elements, unnatural peptides, synthesized organic molecules, naturally occurring organic molecules, nucleic acid molecules, and components thereof. Identification of a peptidomimetic can be accomplished by screening methods incorporating a binding pair and identifying compounds that displace the binding pair. Alternatively, a peptidomimetic can be designed in silico, by molecular modeling of a known protein-protein interaction, for example, the interaction of a phosphopeptide of the invention and a PBD. Desirably, the peptidomimetic will displace one member of a binding pair by occupying the same binding interface. More desirably the peptidomimetic will have a higher binding affinity to the binding interface.

By “Polo-like kinase (PLK) nucleic acid molecule” is meant a nucleic acid, or nucleic acid analog, that encodes a Polo-like kinase polypeptide. For example, a Plk-1 nucleic acid molecule is substantially identical to GenBank Accession Number X73458 (SEQ ID NO: 17) or NM_{—}005030 (SEQ ID NO: 18); a Plk-2/SNK nucleic acid molecule is substantially identical to NM_{—}006622 (SEQ ID NO: 19); a Plk-3 nucleic acid molecule is substantially identical to NM_{—}004073 (SEQ ID NO: 20); a Plx-1 nucleotide sequence is substantially identical to GenBank Accession Number U58205 (SEQ ID NO: 21); and a Polo nucleic acid molecule is substantially identical to GenBank Accession Number AY095028 (SEQ ID NO: 22) or NM 079455 (SEQ ID NO: 23).

By a “Polo-like kinase” is meant a polypeptide substantially identical to a Polo-like kinase amino acid sequence, having serine/threonine kinase activity, and having at least one Polo-box domain consisting of 2 Polo-boxes. Exemplary Polo-like kinase polypeptides include, Plk-1 (GenBank Accession Number NP 005021, SEQ ID NO:1); Plk-2 (GenBank Accession Number NP 006613, SEQ ID NO:4); and Plk-3 (GenBank Accession Number NP_{—}004064, SEQ ID NO:5). Additional Polo-like kinase polypeptides include GenBank Accession Numbers P53350 (SEQ ID NO: 24), and Q07832 (SEQ ID NO: 25).

Structurally, Polo or Polo-like kinases have a unique amino terminus followed by a serine/threonine kinase domain, a linker region, a Polo-box (PB1), a linker sequence, a second Polo-box (PB 2), and a small stretch of 12-20 amino acids at the carboxy terminus (see FIG. 2A).

In desirable embodiments, Polo-like kinases include Saccaromyces cereviseae, CdcS, Schizosaccaromyces pombe, Plo-1, Drosophila melanogaster, Polo, Xenopus laevis, Plx (Plx-1, -2, -3), and mammalian Plk-1, Prk/Fnk, Snk, and Cnk. The Polo-box is approximately 70 amino acids in length and is shown in FIG. 2B (indicated by the bold lines).

By “Polo-like kinase biological activity” is meant any biological activity associated with Polo-like kinases, such as serine/threonine kinase activity. Other biological activities of Polo-like kinases include the localization of the kinase to the centrosomes, spindle apparatus, and microtubular organizing centers (MOCs).

By “polypeptide” is meant any chain of at least two naturally-occurring amino acids, or unnatural amino acids (e.g., those amino acids that do not occur in nature) regardless of post-translational modification (e.g., glycosylation or phosphorylation), constituting all or part of a naturally-occurring or unnatural polypeptide or peptide, as is described herein. Naturally occurring amino acids are any one of the following, alanine (A or Ala), cysteine (C or Cys), aspartic acid (D or Asp), glutamic acid (E or Glu), phenylalanine (F or Phe), glycine (G or Gly), histidine (H, or His), isoleucine (I or Ile), lysine (K or Lys), leucine (L or Leu), methionine (M or Met), asparagine (N or Asn), ornithine (O or Orn), proline (P or Pro), hydroxyproline (Hyp), glutamine (Q or Gln), arginine (R or Arg), serine (S or Ser), threonine (T or Thr), valine (V or Val), tryptophan (W or Trp), or tyrosine (Y or Tyr).

By “peptide” is meant any compound composed of amino acids, amino acid analogs, chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids, amino acid analog, or small and large peptides, including polypeptides.

Polypeptides or derivatives thereof may be fused or attached to another protein or peptide, for example, as a Glutathione-S-Transferase (GST) fusion polypeptide. Other commonly employed fusion polypeptides include, but are not limited to, maltose-binding protein, Staphylococcus aureus protein A, Flag-Tag, HA-tag, green fluorescent proteins (e.g., eGFP, eYFP, eCFP, GFP, YFP, CFP), red fluorescent protein, polyhistidine (6×His), and cellulose-binding protein.

By “phosphopeptide” or “phosphoprotein” means a peptide or protein in which one or more phosphate moieties are covalently linked to serine, threonine, tyrosine, aspartic acid, histidine amino acid residues, or amino acid analogs. A peptide can be phosphorylated to the extent of the number of serine, threonine, tyrosine, or histidine amino acid residues that is present. Desirably, a phosphopeptide is phosphorylated at 4 independent Ser/Thr/Tyr residues, at 3 independent Ser/Thr/Tyr residues, or at 2 independent Ser/Thr/Tyr residues. Most desirably, a phosphopeptide is phosphorylated at one Ser/Thr/Tyr residue regardless of the presence of multiple Ser, Thr, or Tyr residues.

Typically, a phosphopeptide is produced by expression in a prokaryotic or eukaryotic cell under appropriate conditions or in translation extracts where the peptide is subsequently isolated, and phosphorylated using an appropriate kinase. Alternatively, a phosphopeptide may be synthesized by standard chemical methods, for example, using N-α-FMOC-protected amino acids (including appropriate phosphoamino acids). In a desired embodiment, the use of non-hydrolysable phosphate analogs can be incorporated to produce non-hydrolysable phosphopeptides (Jenkins et al., J. Am. Chem. Soc., 124:6584-6593, 2002; herein incorporated by reference). Such methods of protein synthesis are commonly used and practiced by standard methods in molecular biology and protein biochemistry (Ausubel et al., Current Protocols in Molecular Biology, John Wiley & Sons, New York, N.Y., 1994, J. Sambrook and D. Russel, Molecular Cloning: A Laboratory Manual, 3^{rd} Edition, Cold Spring Harbor Laboratory Press, Woodbury N.Y., 2000). Desirably, a phosphopeptide employed in the invention is generally not longer than 100 amino acid residues in length, desirably less than 50 residues, more desirably less than 25 residues, 20 residues, 15 residues. Most desirably the phosphopeptide is 2, 3, 4, 5, 6, 7, 8, 9, or 10 amino acid residues long.

By “substantially identical” is meant a polypeptide or nucleic acid exhibiting at least 75%, but preferably 85%, more preferably 90%, most preferably 95%, or even 99% identity to a reference amino acid or nucleic acid sequence. For polypeptides, the length of comparison sequences will generally be at least 35 amino acids, preferably at least 45 amino acids, more preferably at least 55 amino acids, and most preferably 70 amino acids. For nucleic acids, the length of comparison sequences will generally be at least 60 nucleotides, preferably at least 90 nucleotides, and more preferably at least 120 nucleotides.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software matches similar sequences by assigning degrees of homology to various substitutions, deletions, and other modifications. Conservative substitutions typically include substitutions within the following groups: glycine, alanine, valine, isoleucine, leucine, methionine; aspartic acid, glutamic acid, asparagine, glutamine; serine, threonine; lysine, arginine; and phenylalanine, tyrosine.

By “unnatural amino acid” is meant an organic compound that has a structure similar to a natural amino acid, where it mimics the structure and reactivity of a natural amino acid. The unnatural amino acid as defined herein generally increases or enhances the properties of a peptide (e.g., selectivity, stability, binding affinity) when the unnatural amino acid is either substituted for a natural amino acid or incorporated into a peptide.

Unnatural amino acids and peptides including such amino acids are described in U.S. Pat. Nos. 6,566,330 and 6,555,522.

Other features and advantages of the invention will be apparent from the following description of the desirable embodiments thereof, and from the claims.

### BRIEF DESCRIPTION OF THE DRAWINGS

The application file contains drawings executed in color (FIGS. 10, 11, 12, 14, and 21). Copies of this patent or patent application with color drawings will be provided by the Office upon request and payment of the necessary fee.

FIGS. 1A and 1B depict a novel phospho-motif-based library vs. library screen to identify phosphoserine/threonine binding domains. FIG. 1A depicts a library of phosphothreonine-proline oriented phosphopeptides, biased toward the phosphorylation motifs for cyclin-dependent kinases and MAP kinases and toward the epitope of the monoclonal antibody MPM-2, and immobilized on Streptavidin beads. This library and its unphosphorylated counterpart were screened against 680 pools of in vitro translated ³⁵S-Met labeled proteins. pT denotes phosphothreonine. B represents a biased mixture of the amino acids P, L, I, V, F, M, W. FIG. 1B is a set of four SDS-PAGE/autoradiographs. The WW-domain containing protein Pin1 and a fragment of the mitotic kinase Plk-1, denoted by asterisks, were isolated from two pools as clones that associated preferentially with the phosphorylated form of the immobilized peptide library. In each panel, the first lane shows 10% of the input radiolabeled protein pool, while the second and third lanes show binding of proteins within this pool to the phosphorylated and unphosphorylated immobilized libraries, respectively. Identification of Pin1 and Plk1 occurred through progressive subdivision of their respective pools to single clones (panels on right). Arrowheads indicate partial translation or proteolytic breakdown products of Plk1 that exhibit more dramatic phospho-discrimination than the full-length transcript of the isolated Plk1 fragment, suggesting that the full-length transcript likely contains a smaller discrete phospho-binding domain.

FIG. 2A is a schematic diagram showing various C-terminal truncations of Plk-1, translated in vitro, and assayed for selective binding to the phosphorylated peptide library of FIG. 1A over its unphosphorylated counterpart. The two shaded regions in the C-terminus of Plk-1 correspond to its polo boxes (PB1 and PB2) as defined by Pfam. Truncated constructs were designed according to boundaries of sequence homology within the polo-like kinase family rather than boundaries of the Pfam-delineated polo boxes. Clone 407-C6 is the fragment of Plk-1 isolated from the screen depicted in FIGS. 1A and B.

FIG. 2B shows an amino acid sequence alignment of the C-terminal noncatalytic region of human Plk-1 (SEQ ID NO: 77), Xenopus Plx-1 (SEQ ID NO: 78), and Drosophila Polo (SEQ ID NO: 79). Bold lines indicate the designated polo boxes (PB1 and PB2) of Plk-1 as defined by Pfam.

FIGS. 3A-3D are histograms showing the binding ratios of the Plk-1 polo-box domain (PBD). The Polo-box Domain (PBD, residues 326-603) of Plk-1 was expressed as a GST fusion protein, immobilized on Glutathione-agarose beads, and incubated with phosphothreonine/serine-oriented degenerate peptide libraries consisting of the sequences MAXXXXpTPXXXXAKK (SEQ ID NO: 26) (3A), MAXXXXpSPXXXXAKK (SEQ ID NO: 27) (3B), MAXXXXSpTXXXXAKK (SEQ ID NO: 28) (3C), or MAXXXXSpSXXXXAKK (SEQ ID NO: 29) (3D) where X indicates all amino acids except Cys. Following extensive washing, bound peptides were eluted and sequenced. The bar graphs show the relative abundance of each amino acid at a given cycle of sequencing compared to its abundance in the starting peptide library mixture. The Plk-1 PBD selects for serine in the pThr/Ser-1 position strongly (5.9 or 8.1) and for proline in the pThr/Ser+1 position moderately (1.6 or 1.8).

FIG. 3E is an autoradiograph. Pin1 (3E) shows an absolute requirement for proline in the pThr+1 position, whereas the PBD of Plk-1 does not. Full-length Pin1 and the PBD (residues 326-603) of Plk-1 were translated in vitro in the presence of ³⁵S-methionine and tested for binding to four immobilized peptide libraries that differed by phosphorylation status and/or the presence of proline in the pThr+1 position.

pTP=biotin-ZGZGGAXXBXpTPXXXXAKKK (SEQ ID NO: 30),
TP=biotin-ZGZGGAXXBXTPXXXXAKKK (SEQ ID NO: 31),
pT=biotin-ZGZGGAXXXXpTXXXXXAKKK (SEQ ID NO: 32),
T=biotin-ZGZGGAXXXXTXXXXXAKKK (SEQ ID NO: 33),
where pT is phosphothreonine, Z indicates aminohexanoic acid, X denotes all amino acids except Cys, and B is a biased mixture of the amino acids P, L, I, V, F, M, W.

FIGS. 4A-D shows isothermal titration calorimetry results. These results show that Plk1 PBD binds its optimal phosphopeptide ligand with high affinity and high specificity.

FIG. 4E is a table. Isothermal titration calorimetry (ITC) was used to determine binding constants (K_{d}) for the association of the Plk-1 PBD (residues 326-603) with its optimal phosphopeptide ligand and with nine mutated versions of this peptide. All observed binding stoichiometries were consistent with a 1:1 complex of PBD and phosphopeptide. N.D.B indicates no detectable binding by ITC for a Plk-1 PBD concentration of at least 150 μM. pT, pS, and pY denote phosphothreonine, phosphoserine, and phosphotyrosine, respectively. The following sequences are shown: PoloBoxtide-optimal (SEQ ID NO:3), PoloBoxtide-8T (SEQ ID NO:34), PoloBoxtide-8pS (SEQ ID NO:65), PoloBoxtide-8pY (SEQ ID NO:66), PoloBoxtide-7V (SEQ ID NO:67), PoloBoxtide-7A (SEQ ID NO:68), PoloBoxtide-7G (SEQ ID NO:69), PoloBoxtide-7C (SEQ ID NO:70), PoloBoxtide-7T (SEQ ID NO:71), and PoloBoxtide-9N (SEQ ID NO:72).

FIG. 5A upper panel shows a FACS (fluorescence activated cell sorter) trace of human cells used in the pull-down assays shown below. The upper left panel shows the FACS profile of the cells arrested with aphidocolin in G1 (so the total DNA content is 1N where N=the normal amount of DNA in a diploid human cell) and verifies that the cells were in G1. The right trace shows the FACS profile of the cells arrested with nocadozole to trap them in G2/M, and shows that their DNA content is 2N, verifying that they are arrested in G2/M. FIG. 5A (lower panel) and 5B are immunoblots showing that the Plk-1 PBD associates with mitotic phosphoproteins in HeLa cells. Lysates from HeLa cells, arrested at interphase with aphidicolin or in G2/M with nocodazole, were incubated with GST, GST-Pini, and the GST-Plk-1 PBD (residues 326-603; FIG. 5A). Mitotic phosphoproteins co-precipitated with these GST fusions were detected by blotting with the pSer-Pro specific monoclonal antibody MPM-2. Interaction of the GST-Plk-1 PBD (residues 326-603) with mitotic phosphoproteins from nocodazole-arrested HeLa cells was disrupted by pre-incubation of GST-Plk-1 PBD with its optimal phosphopeptide ligand, MAGPMQ-S-pT-P-LNGAKK (SEQ ID NO: 2) (PoloBoxtide-optimal), but not with an unphosphorylated equivalent peptide, MAGPMQ-S-T-P-LNGAKK (SEQ ID NO:34) (PoloBoxtide-8T), nor a phosphopeptide whose serine at pThr-1 was mutated to valine (PoloBoxtide-7V; FIG. 5B).

FIGS. 6A, 6C, and 6D are immunoblots showing that Plk-1 PBD interacts with Thr₁₃₀ of mitosis-dependent phosphorylated Cdc25C from HeLa cells. FIG. 6A is an anti-CDC25 western blot on lysates from HeLa cells arrested in interphase with aphidicolin or in G2/M with nocodazole, incubated with a GST fusion of the Plk-1 PBD (residues 326-603). Endogenous Cdc25C from mitotic lysates was precipitated with GST-Plk-1 PBD and detected by anti-Cdc25C (Santa Cruz Biotechnology). Interaction of GST-Plk-1 PBD with Cdc25C was disrupted as in FIG. 5B by pre-incubation of GST-Plk-1 PBD with its optimal phosphopeptide ligand (PoloBoxtide-optimal) but not with the PoloBoxtides-8T or -7V. FIG. 6B is a sequence alignment showing that a consensus motif for the Polo-box Domain of Plk-1 is conserved between human (SEQ ID NO: 80) and Xenopus Cdc25C (SEQ ID NO: 81). T130 and T138 of human and Xenopus Cdc25C, respectively, are known to be phosphorylated during mitosis (FIG. 6B). Lysates were prepared from HeLa cells transfected with either wild type, T130A, or S129V HA-Cdc25C (human), arrested in G2/M with nocodazole, and normalized for equal loading of the mitotically up-shifted form. Interaction of GST-Plk-1 PBD (residues 326-603) with mitotically phosphorylated Cdc25C from these lysates was detected by pull-down with glutathione beads, separation by 11.4% SDS-PAGE and anti-HA blotting (FIG. 6C). FIG. 6D shows lysates, analyzed by 9% SDS-PAGE to enhance separation of the hyper-phosphorylated (P) form of Cdc25C from partially phosphorylated and unphosphorylated (U) forms.

FIG. 7A is a set of micrographs visualized using fluorescence microscopy. FIG. 7B is a histogram showing the ratio of centrosomal localization by the GST-PBD relative to centrosomal γ-tubulin. U2OS cells were arrested in G2/M with nocodazole and then incubated with 4 μM GST-Plk-1 PBD (residues 326-603) in cell permeabilization buffer containing 1 U/ml Streptolysin-O in the presence of no peptide (upper panel), 250 μM of the optimal phosphopeptide (optimal, middle panel), or 250 μM of the corresponding unphosphorylated analogue (8T, lower panel). Following incubation, the cells were washed extensively, fixed with paraformaldehyde, extracted with Triton X-100, immunostained for GST and γ-tubulin, and counterstained with DAPI to visualize the nucleus. Overlap of the GST (Alexa Fluor 488) and γ-tubulin (Texas Red) signals is shown in the merged figure in the far right column (FIG. 7A). The ratio of centrosomal localization by the GST-PBD relative to centrosomal γ-tubulin levels is shown in FIG. 7B.

FIG. 8 is a schematic diagram showing a model for 2-step activation of Cdc25 and Cdc2/Cyclin B auto-activation through Plk-1. Phosphorylation of a few molecules of Cdc25, either by a small amount of de-repressed Cdc2/Cyclin B or another proline-directed kinase early in mitosis, primes those Cdc25 molecules for binding of Plk-1 through its PBD. Activation of the Plk-1 kinase domain by Plkkl generates the first wave of Cdc25 activation, dephosphorylating more Cdc2/Cyclin B, which, in turn, phosphorylates additional Cdc25 molecules for interaction with the Plk-1 PBD. The net result is a positive feedback loop for Cdc2/Cyclin B activation (circled).

FIG. 9A is a table showing the conservative mutations at the pT-1 serine that abolish Plk1 PBD/peptide binding in solution. Isothermal titration calorimetry was used to determine binding affinities. The Plk1 PBD (residues 326-603) was expressed in E. coli as a GST fusion, purified on glutathione agarose, proteolytically digested from GST, and further purified by anion exchange chromatography. N.D.B. indicates no detectable binding for a Plk1 PBD concentration of at least 150 μM. pT denotes phosphothreonine. Throughout FIGS. 9A and 9B, the domains are depicted as follows: kinase: white; PC: gray; PB1: red; PB2: blue; The following sequences are shown: PoloBoxtide-optimal (SEQ ID NO:3), PoloBoxtide-7A (SEQ ID NO:68), PoloBoxtide-7G (SEQ ID NO:69), PoloBoxtide-7C (SEQ ID NO:70), and PoloBoxtide-7T (SEQ ID NO:71).

FIG. 9B is a filter array that shows binding of GST-Plk1 PBD (residues 326-603) to peptide spots, comprising single point mutants of the Plk1 PBD optimal phosphopeptide (right column). Bound GST-Plk1 was detected by blotting with HRP-conjugated anti-GST antibody.

FIG. 10A is a schematic diagram showing the boundaries of the PBD by limited proteolysis. Domain architecture of full-length Plk1 and stable fragments (left) are shown together with the time-course of V8 protease digestion (right). Molecular weight and amino acid boundaries of the limiting domain were determined by mass spectroscopy.

FIG. 10B is a schematic diagram showing the Polo-box 1 and Polo-box 2 β₆α structures, colored as in (A), are shown superimposed.

FIG. 10C is a RIBBONS representation (Carson, 1991) of the structure of the Plk1 PBD in complex with a phosphothreonine-containing peptide shown as a ball and stick representation in yellow. The Polo-boxes and Polo-cap region are colored as in (A). The phosphopeptide binds at one end of a pocket formed between the two polo boxes.

FIG. 11A shows a structure-based sequence alignment of the Polo-box Domain family. The following sequences are shown: HsPlk1 (SEQ ID NO: 82), MmPlk1 (SEQ ID NO: 83), RnPlk1 (SEQ ID NO: 84), CePlk1 (SEQ ID NO: 85), DmPolo (SEQ ID NO: 86), XlP1X1 (SEQ ID NO: 87), HpPlk1 (SEQ ID NO: 88), HsPlk2 (SEQ ID NO: 89), MmPlk2 (SEQ ID NO: 90), RnPlk2 (SEQ ID NO: 91), CePlk2 (SEQ ID NO: 92), XlP1x2 (SEQ ID NO: 93), HsPlk3 (SEQ ID NO: 94), MmPlk3 (SEQ ID NO: 95), RnPlk3 (SEQ ID NO: 96), XlP1X3 (SEQ ID NO: 97), SpPlol (SEQ ID NO: 98), and ScCdc5 (SEQ ID NO: 99). Residues with 100% conservation are shaded purple while highly conserved residues are shaded cyan.

FIG. 11B is an image of the molecular surface of the PBD based on the structure determined by X-ray crystallography. The surface positions corresponding to the conserved residues are colored as in FIG. 11A. The most highly conserved residues within the Plk1 PBD are located exclusively on the peptide-binding face of the PBD. The most highly conserved residues within the Plk1 PBD are located exclusively on the peptide binding face of the PBD. The coloring scheme is as in 11A.

FIG. 11C is a schematic diagram depicting the electrostatic potential of the PBD phosphopeptide pocket, calculated using GRASP (Nicholls et al., 1991), with the phosphopeptide superimposed in stick representation (oxygen atoms, red; nitrogen atoms, blue). Negative potential of the PBD surface is colored red and positive potential blue.

FIG. 11D is a schematic representation of the interactions between the phosphopeptide (blue) and the Plk1 PBD. Hydrogen bonds, van der Waals interactions, and water molecules are denoted by dotted lines, purple crescents, and green circles, respectively.

FIG. 11E is a schematic representation of direct and indirect hydrogen bonds (dotted lines) between the phosphate and the Plk1 PBD. Hydrogen bond lengths are given in angstroms.

FIG. 12A is a schematic diagram showing a comparison of the β-sandwich folds of the Plk1 PBD and the Sak polo-box dimer. Tertiary structures are shown on the top together with secondary structure topology (triangles, β strands; rectangles, α-helices) on the bottom. PB1 and PB2 of Plk1 are denoted by red and purple colors, respectively, while the Pc of Plk1 is shown in green. Polo-boxes from separate Sak molecules within the dimer are likewise denoted by red and purple. The Sak β sandwich involves strand swapping between separate polo-boxes within the dimer.

FIG. 12B is a sequence alignment of the Polo-boxes from Plk1 (HsPlk1_pb1, SEQ ID NO: 100; and HsPlk1_pb2, SEQ ID NO: 101) and Sak (SEQ ID NO: 102). Plk1 has a β6α secondary topology while Sak has a circularly altered β5αβ topology. β-sheet and α-helix notation follows PB1; the corresponding elements for PB2 are β7 through β12 and αC. A conserved salt-bridging interaction initially observed in the Sak structural analysis (Leung et al., Nat. Struct. Biol. 9:719-724, 2002) is shown by the blue bracket. Conserved non-polar residues are highlighted in blue and residues conserved between Sak and at least one of the Plk1 PBDs are boxed.

FIG. 13A is an autoradiograph. Wild type and mutant Plk1 PBD (residues 326-603) were translated in vitro in the presence of ³⁵S-methionine and examined for binding to an immobilized pThr-Pro-oriented library and its unphosphorylated counterpart. pTP=biotin-ZGZGGAXXBXpTPXXXXAKKK SEQ ID NO: 30, TP=biotin-ZGZGGAXXBXTPXXXXAKKK SEQ ID NO: 31, where pT is phosphothreonine, Z is aminohexanoic acid, X is all amino acids except Cys, and B denotes a biased mixture of the amino acids P, L, I, V, F, M, W.

FIG. 13B is a diagram showing isothermal titration calorimetry results. A H538A/K540M mutation of the Plk1 PBD abolishes binding to its optimal phosphopeptide as measured by isothermal titration calorimetry.

FIG. 13C is a Western blot showing that mutation of the H538/K540 pincer disrupts interaction of the isolated Plk1 PBD with Cdc25 in vivo. HeLa cells were transfected with wild type and mutant versions of a His-Xpress-tagged Plk1 PBD construct (residues 326-603) or with a control Plk1PBD construct lacking the second Polo-box (residues 326-506) and arrested in G2/M with nocodazole. The Plk PBD was pulled down with Ni²⁺ beads and bound endogenous proteins analysed by SDS-PAGE and blotted for Cdc25.

FIG. 13D is a Western blot showing that mutation of the H538/K540 pincer in the Plk1 PBD disrupts interaction of full-length Plk1 with Cdc25 in vivo. HeLa cells were transfected with wild type and mutant versions of full-length myc-tagged Plk1 and arrested in G2/M with nocodazole. Plk-myc was immunoprecipitated with anti-myc-conjugated beads and Cdc25 binding to Plk1 analyzed as in 13C.

FIG. 14 is a series of photomicrographs showing that mutation of the H538/K540 pincer sequence abolishes centrosomal localization of the Plk1 PBD in HeLa Cells. U2OS cells were arrested in G2/M with nocodazole and then incubated with 4 μM wild-type or mutant GST-Plk1 PBD (residues 326-603) in cell permeabilization buffer containing 1 U/ml Streptolysin-O. Following incubation, the cells were washed extensively, fixed with paraformaldehyde, extracted with Triton X-100, immunostained for GST and γ-tubulin, and counterstained with DAPI to visualize the nucleus. Overlap of the GST (Alexa Fluor 488) and γ-tubulin (Texas Red) signals is shown in the merged figure in the far right column.

FIG. 15 is a series of diagrams showing the results of FACS analysis. HeLa cells were transfected with wild type and mutant GFP-tagged Plk1 (residues 326-603) for 32 hours. Cells were harvested, stained with Hoechst 33342, and analyzed by FACS to determine DNA content in the total cell populations (left panels). Similar analysis limited to the transfected cell population was performed by gating only on the GFP expressing cells (right panels). G2/M population percentages are averages from three independent experiments.

FIG. 16A is a Western blot that phosphopeptide binding by full-length Plk1 is reduced relative to that for the isolated Plk1 PBD. Approximately 10% of input full length Plk1 (residues 1-603) interacted with an immobilized pThr-Pro oriented library with slight preference over the unphosphorylated library analogue. The phosphorylation-dependent component of binding arose from the PBD, as it was eliminated by mutation of the His538/K540M pincer. In contrast, phosphopeptide binding by the isolated PBD (FIG. 13A) was 10-fold greater and considerably more phospho-dependent.

FIG. 16B is a graph showing that the optimal PBD phosphopeptide stimulates full-length Plk1 kinase activity. GST-Plk1 (prepared in SF9 cells) was preincubated without peptide (closed circles), with 250 μM of the optimal PBD phosphopeptide (open squares) or with 250 μM of the non-phosphorylated optimal peptide counterpart (closed squares) for 5 minutes at room temperature prior to initiating the kinase reaction by addition of ATP. [32P]-incorporation into casein was determined by SDS-PAGE electrophoresis, autoradiography, and densitometry. Pre-incubation with the optimal PBD phosphopeptide ligand enhanced the rate of casein phosphorylation by Plk1 by a factor of 2.6 as determined from three independent experiments.

FIG. 16C is a schematic diagram depicting a model for Plk1 regulation by the PBD. PB1 and PB2 are shaded orange, kinase domain cyan, phosphopeptide purple with phosphate in red. Inhibitory interactions between the PBD and the kinase domain in the basal state (left) are relieved by phosphopeptide binding, which may also stabilize association of the two Polo-boxes (right).

FIG. 17A is an autoradiograph showing the identification of phosphoSer/Thr-binding domains using an ATM/ATR-motif library. An oriented (pSer/pThr) phosphopeptide library, biased toward the phosphorylation motifs for ATM/ATR kinases, was immobilized on Streptavidin beads. This phosphopeptide library [pSQ=biotin-ZGZGGAXXXB(pS/pT)QJXXXAKKK (SEQ ID NO:35)] and its non-phosphorylated counterpart were screened against in vitro translated ³⁵S-Met labeled proteins. (pS/pT) denotes 50% phosphoserine and 50% phosphothreonine; Z indicates aminohexanoic acid; B represents a biased mixture of the amino acids A, I, L, M, N, P, S, T, V; and J represents a biased mixture of 25% E, 75% X, where X denotes all amino acids except Arg, Cys, His, and Lys. PTIP, denoted by arrow, was isolated from pool EE11 as a clone that associated preferentially with the phosphorylated form of the immobilized peptide library. In each panel, the first and second lanes show binding of proteins within the pool to the phosphorylated and non-phosphorylated libraries, respectively. Identification of PTIP occurred through progressive subdivision of the EE11 pool to a single clone (panel on right denoted by asterisk). Longer exposures revealed partial translation or proteolytic breakdown products of PTIP that also exhibit phospho-discrimination, suggesting that the full-length transcript likely contains a smaller discrete phospho-binding domain. The uppermost band is a fusion artifact of PTIP with vector sequences resulting from translation initiation at an upstream ATG in the vector.

FIG. 17B is an autoradiograph showing deletion mapping of the phospho-binding domain of PTIP. Truncations of PTIP were translated in vitro and assayed for selective binding to the phosphorylated peptide library as in FIG. 17A. Shaded regions in the C-terminus of PTIP correspond to its BRCT domains. Truncation constructs were designed according to boundaries of sequence homology within the BRCT domain, boundaries from sequence alignments, and from the Pfam-delineated BRCT domains (Bateman et al., Nucleic Acids Res 27: 260-2, 1999).

FIG. 18A is an autoradiograph. PTIP, BRCA1, MDC1, 53BP1 and Rad9 tandem BRCT domains were translated in vitro in the presence of 35S-methionine and tested for binding to immobilized phosphopeptide and non-phosphopeptide libraries as described in FIG. 17A. The peptide libraries used were pSQ as defined in FIG. 17A. pS=biotin-ZGZGGAXXXXpSXXXXXAKKK SEQ ID NO: 36; pT=biotin-ZGZGGAXXXXpTXXXXXAKKK SEQ ID NO: 32, where pS is phosphoserine, pT is phosphothreonine, Z indicates aminohexanoic acid, and X denotes all amino acids except Cys. Both PTIP and BRCA1 tandem BRCT domains display stronger binding to the pSQ and pS libraries as compared to the non-phospho libraries. Domain boundaries: PTIP as indicated in FIG. 1 (SEQ ID NO: 15); BRCT1 and 2: amino acids 1634-1863 of SEQ ID NO: 12; BRCT1 alone: amino acids 1634-1751 of SEQ ID NO: 12; BRCT2 alone: 1725-1863 of SEQ ID NO: 12; MDC1: amino acids 1880-2089 of SEQ ID NO: 37(NP_{—}055456.1); 53BP1: amino acids 1700-1972 of SEQ ID NO: 38 (NP 005648.1); Rad9: amino acids 1025-1309 of SEQ ID NO: 39 (NP_{—}010503.1).

FIGS. 18B and C are autoradiographs showing that the PTIP and BRCA1 BRCT domains show strong selection for Phe at the (pSer/pThr)Gln +3 position (7.0 or 7.5), respectively. Tandem BRCT domains of PTIP and BRCA1 were immobilized as glutathione-S-transferase (GST) fusion proteins on glutathione beads and incubated with non-biotinylated versions of the oriented degenerate phosphopeptide libraries described in FIG. 17A (XXXB-pS/pT-QJXXX, SEQ ID NO: 118). Following extensive washing, bound peptides were eluted and sequenced. Bar graphs show the relative abundance of each amino acid at a given cycle of sequencing compared to its abundance in the starting peptide library mixture, as described (Yaffe et al., Methods Enzymol 328:157-70, 2000).

FIGS. 18D, 18E, 18F, and 18G show binding of GST-PTIP and BRCA1 tandem BRCT domains to a filter array of peptide spots, comprising single point mutants of the optimal BRCT domain phosphopeptide (left column). Bound GST-BRCT domains were detected by blotting with HRP-conjugated anti-GST antibody. The resulting consensus binding motif is indicated in the right column, SEQ ID NO: 119, SEQ ID NO: 120, SEQ ID NO: 121, and SEQ ID NO: 122 for FIGS. 18D, 18E, 18F, and 18G, respectively; wherein X denotes no dominant selection, φ denotes residues with aliphatic or aromatic side chains, and letters enclosed in square brackets are specifically de-selected. The top row indicates the amino acid that was substituted for the optimal amino acid. Substitution of pSer for pThr enhanced binding for both PTIP and BRCA1 BRCT domains, consistent with the ITC results. Substitution of pTyr for pThr eliminated binding altogether, verifying that tandem BRCT domains are pSer/pThr-specific binding modules. Replacement of pThr with Thr, Ser or Tyr abrogated tandem BRCT domain binding. The pTQ oriented blots on the left show strong selection at several positions for both PTIP and BRCA1 BRCT domains; especially for Phe in the +3 position in agreement with the oriented peptide library screening data. The pS oriented blots on the right show that the +3 position is the most important position for peptide selection.

FIG. 19A is a Western blot. Lysates from U2OS cells were obtained prior to and 2 hours after the cells were exposed to 10 Gy of ionizing radiation (IR). The lysates were incubated with GST-PTIP tandem BRCT domains, and bound proteins were detected by blotting with the anti-ATM/ATR phosphoepitope motif antibody. Interaction of the PTIP BRCT domains with these phosphoproteins from IR treated cells was disrupted by pre-incubation with the pSQ peptide library, but not with the SQ peptide library or the pTP library.

FIG. 19B is a Western blot showing that the interaction of the PTIP BRCT domains with DNA damage induced phosphoproteins from IR treated U2OS cells was disrupted by pre-treating the cells with caffeine (25 mM) prior to IR exposure or by pre-incubating the beads with an optimal BRCT-binding peptide (BRCTtide-opt), but not by preincubating the beads with the peptide's non-phosphorylated counterpart (BRCTtide-7T).

FIG. 19C is a Western blot showing that tandem BRCT domains of PTIP interact with 53BP 1 following DNA damage. Endogenous 53BP 1 from IR treated U2OS cells was precipitated with GST-PTIP tandem BRCT domains and detected by incubating with an anti-53BP1 antibody. Interaction of GST-PTIP tandem BRCT domains with HA-tagged 53BP1, was then detected by anti-HA blotting. This interaction was abolished by treating the lysates with lambda phosphatase, by pre-incubating the beads with an optimal BRCT-binding peptide (BRCTtide-opt), but not with its non-phosphorylated counterpart (BRCTtide-7T), or by preincubating the beads with the pSQ library, but not by preincubating with the SQ library or the pTP library. Treatment of the cells with 25 mM caffeine also disrupted the interaction.

FIG. 19D is a Western blot. Lysates from U2OS cells 2 hours following IR were incubated with GST-BRCA1 tandem BRCT domains. DNA damage-induced phosphoproteins were detected by blotting with the anti-ATM/ATR phosphoepitope motif antibody. The interaction of the GST-BRCA1 tandem BRCT domains with the phosphoproteins were disrupted as in panel B. These results show that tandem PTIP and BRCA1 BRCT domains associate with DNA damage-induced phosphoproteins through their phosphopeptide-binding pockets.

FIGS. 20A-C are photomicrographs showing immunofluorescence in U2OS cells demonstrating that full length PTIP forms DNA damage induced foci and co-localizes with (pSer/pThr)-Gln proteins, 53BP1, and γ-H2AX. FIG. 20A shows U2OS cells transfected with a full length PTIP-GFP construct (PTIP-FL residues 1-757). FIG. 20B shows U2OS cells transfected with a PTIP deletion construct in which the last two BRCT domains were removed (PTIP-ABRCT, residues 1-550). FIG. 20C shows U2OS cells transfected with a PTIP construct containing only the last two BRCT domains (BRCT)₂, residues 550-757). In FIGS. 20A-20C, 24 hours following transfection cells were either treated with 10 Gy of ionizing radiation or mock irradiated, allowed to recover for 2 hours, stained, and analyzed by immunofluorescence microscopy.

FIGS. 21A and B are photomicrographs showing immunofluorescence in U2OS cells demonstrating that caffeine attenuates recruitment of PTIP to DNA damage foci in response to ionizing radiation. U2OS cells transfected with full-length PTIP-GFP cDNA were mock treated or pretreated with 10 mM caffeine for 70 minutes before exposure to 10Gy ionizing radiation. (A) In response to IR, mock-treated U2OS cells formed nuclear foci containing PTIP (in green) and H2AXp (in red); these two proteins co-localize at sites of DNA damage (merge). (B) In response to IR, caffeine treated U2OS cells formed reduced numbers of nuclear foci; PTIP was mislocalized and did not form discrete nuclear foci (in green) and there were reduced numbers of H2AXp (in red) containing foci; pretreatment with caffeine effectively abolished co-localization of PTIP and H2AXp (merge).

FIG. 22 shows the PTIP amino acid sequence (SEQ ID NO: 15).

FIG. 23 shows the PTIP nucleic acid sequence (SEQ ID NO: 13).

FIG. 24 shows the BRCA1 amino acid sequence (SEQ ID NO: 12).

FIGS. 25A-C show the BRCA1 nucleic acid sequence (SEQ ID NO: 11).

FIG. 26 shows the MDC1 amino acid sequence (SEQ ID NO: 37).

FIGS. 27A-C show the MDC1 nucleic acid sequence (SEQ ID NO: 40).

FIG. 28 shows the 53BP1 amino acid sequence (SEQ ID NO: 38).

FIGS. 29A-C show the 53BP1 nucleic acid sequence (SEQ ID NO: 41).

FIG. 30 shows the Rad9 amino acid sequence (SEQ ID NO: 39).

FIGS. 31A-B show the Rad9 nucleic acid sequence (SEQ ID NO: 42).

### DESCRIPTION OF THE INVENTION

The present invention features a method for identifying kinase targets, an exemplary kinase target, the Polo box domain of the Polo-like kinase, and exemplary peptide mimetics that interfere with signaling by the Polo-like kinase.

We have developed a proteomic approach that allows us to identify virtually any peptide-binding domain by simultaneously screening a polypeptide expression library with a biased peptide library. We have used this method to identify, for example, targets downstream of kinases in signaling pathways. This strategy involves using an immobilized library of partially degenerate phosphopeptides, biased toward a kinase phosphorylation motif, to isolate interacting effector proteins targeted by substrates of that kinase. Using this approach for cyclin-dependent kinases, we identified the Polo-box Domain (PBD) of the mitotic kinase Plk-1 as a phosphoserine/threonine binding domain. Polo-like kinases (Plks) perform crucial functions in cell-cycle progression and multiple stages of mitosis. Plks are characterized by the presence of a C-terminal non-catalytic region containing two tandem Polo-boxes, termed the Polo-box domain (PBD).

In addition, we have discovered that the PBDs of human, Xenopus, and yeast Plks all recognize similar phosphoserine/threonine-containing motifs. The 1.9 Å X-ray structure of a human Plk1 PBD-phosphopeptide complex shows that the Polo-boxes β₆α structures. They associate to form a novel 12-stranded β-sandwich domain, to which the phosphopeptide-binds within a conserved, positively-charged cleft located at the edge of the Polo-box interface. Mutations designed to specifically disrupt phosphodependent interactions abolish cell-cycle dependent localization and provide compelling phenotypic evidence that PBD-phospholigand binding is necessary for proper mitotic progression. In addition, phosphopeptide-binding to the PBD stimulates kinase activity in full-length Plk1, suggesting a conformational switching mechanism for Plk regulation and a dual functionality for the PBD. Together, our data reveal a central role for PBD-phosphoprotein interactions in many, if not all, cellular functions of Plks. This finding provides a structural explanation for how Plk-1 localizes to specific sites within cells in response to Cdk phosphorylation at those sites.

Activation of signaling cascades in eukaryotic cells involves the directed assembly of protein-protein complexes at specific locations within the cell. This process is controlled by protein phosphorylation on serine, threonine and/or tyrosine residues that directly or indirectly regulate protein-protein interactions, often through the actions of modular binding domains. Historically, studies of phospho-binding domains have focused on SH2 and PTB domains, which bind to specific phosphotyrosine-containing sequence motifs. Until recently, it was thought that phosphorylation of proteins on serine and threonine residues was not responsible for direct interactions with modular binding domains but instead induced conformational changes to regulate function. However, a number of domains (14-3-3 proteins, FHA domains, WD40 repeats of F-box proteins, MH2 domains and the WW domain of the prolyl isomerase Pin1) have been identified that bind directly to short phosphoserine or phosphothreonine-containing sequences to control cell cycle progression, coordinate the response to DNA damage, and regulate apoptosis.

The vast majority of intracellular proteins are phosphorylated on serine or threonine residues at some point during their lifetime. Furthermore, known phosphoserine/threonine binding domains comprise a diverse structural group, demonstrating that many divergent tertiary folds have acquired a phospho-dependent binding function through evolution. Approximately one-third of the modular protein domains identified by Pfam and SMART on the basis of sequence homology have no known function. Our technique enables the identification of additional phosphopeptide binding modules that target serine/threonine residues.

### 2×2 Biased Library Screening

To design a general proteomic screen capable of identifying novel phosphoserine/threonine binding modules, we took advantage of the observation that protein kinases and phosphopeptide binding domains seem to have co-evolved to recognize overlapping sequence motifs (Yaffe et al., Nat. Biotechnol. 19:348-353, 2001; Obata et al., J. Biol. Chem. 275:36108-36115, 2000). For example, the basophilic protein kinase, Akt, phosphorylates substrates at sites that contain the core motif RXRSX[S/T] (SEQ ID NO: 43) and 14-3-3 proteins bind to a subset of these phosphorylated sites that have the optimal motif RSX[pS/pT]XP (SEQ ID NO: 44). Cyclin-dependent kinases (Cdks) phosphorylate substrates at [S/T]PXR (SEQ ID NO: 45) motifs, and the WW domain of the proline isomerase Pin1 recognizes the phosphorylated forms of these [pS/pT]P sites to mediate isomerization of the proline residue. Importantly, this apparent overlap between kinase and phospho-binding motifs is not perfect. Instead, limited overlap allows combinatorial interactions between substrates of particular kinases and downstream binding modules.

Our motif-based strategy for identifying pSer/Thr-binding domains involved biasing a library of partially degenerate phosphopeptides towards the phosphorylation motif of a kinase and then using an immobilized form of this library as bait in a screen for interacting proteins translated in vitro from a cDNA library.

Using a library of phosphopeptides biased towards motifs phosphorylated by cyclin-dependent kinases (Cdks), we identified the C-terminal Polo-box containing region of the human Polo-like kinase, Plk-1, as a specific phosphopeptide recognition module. It has been previously shown that this non-catalytic region is critical both for Polo kinase subcellular localization and for proper mitotic progression in yeast and human cells. Our findings provide the first description of a biochemical mechanism through which Plk-1 performs these essential mitotic functions. Furthermore, the identification of the conserved Plk-1 PBD as the latest member of the growing superfamily of pSer/Thr-binding domains suggests that phospho-specific docking may be a general mechanism for Ser/Thr kinase signaling in eukaryotic biology.

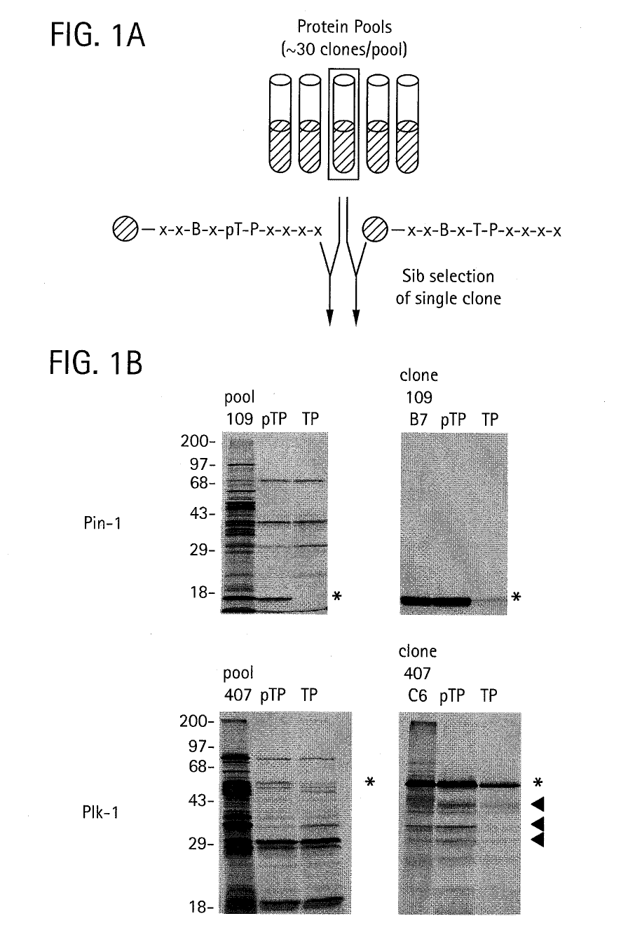

To identify pSer/Thr-binding domains involved in cell cycle regulation, we designed a pThr-Pro-oriented peptide library biased to resemble the motif that would be generated by the action of cyclin-dependent kinases and MAP kinases, as well as that recognized by the mitotic phosphoprotein-specific monoclonal antibody MPM-2, whose pSer/Thr-binding motif we had determined previously (Yaffe et al., Science 278:1957-1960, 1997). The library was constructed with a flexible linker and an N-terminal biotin tag, allowing an immobilized form of this library to be used as bait in an interaction screen against a library of proteins produced by in vitro expression cloning (Lustig et. al., Methods Enzymol 283:83-99, 1997; FIG. 1A).

This library vs. library screening approach is the reverse of a traditional peptide library screen in which a single purified domain is assayed against a degenerate peptide library to reveal the optimal binding motif. In the approach presented here, a degenerate but motif-biased peptide library is used to screen for novel binding domains. By using a collection of peptides biased towards the motif of a protein kinase superfamily, the screen casts a larger net than would be possible if only a single peptide were used as bait. To control for phospho-independent peptide binding, an identical library was constructed with Thr substituted for the fixed pThr residue (FIG. 1A).

The pThr-Pro-oriented peptide library, and its non-phosphorylated Thr-Pro library counterpart were immobilized on Streptavidin beads and screened in parallel against 680 individual pools of in vitro translated [³⁵S]-labeled proteins. Each pool contains ˜30 radiolabeled proteins/pool that are detectable by SDS-PAGE/autoradiography (FIG. 1B, “pool” lanes). As shown in FIG. 1B, proteins produced by in vitro translation often failed to bind either library at all or bound more strongly to the non-phosphorylated peptide library-containing beads. However, we identified 7 distinct pools containing radiolabeled translation products that bound preferentially to the pThr-Pro library compared with the Thr-Pro library (asterisks in FIG. 1B).

Plasmid pools containing these positively scoring hits were progressively subdivided and re-screened for phospho-binding until individual clones were isolated and sequenced. Of the 7 positive clones, 3 were successfully recovered, two of which are reported here. One of the clones, 109-B7, was found to encode the prolyl isomerase Pin1, which is known to bind and isomerize pThr-Pro motifs recognized by the monoclonal antibody MPM-2. Its isolation, therefore, validated the feasibility of our screening approach.

A second positively scoring hit, clone 407-C6, was found to encode the C-terminal 80% of the mitotic kinase Plk-1 (polo-like kinase-1, amino acids 95-603). This clone was missing critical components of the Plk-1 kinase domain, including the glycine rich loop (amino acids 60-66) and the invariant lysine (K82), implying that phosphopeptide binding was independent of Plk-1 kinase activity. Phospho-specific binding by the full-length transcript of this incomplete Plk-1 clone was less pronounced than binding by Pin1 (FIG. 1B). Partial translation products or proteolytic breakdown fragments arising from this clone (FIG. 1B, arrowheads) showed strong discrimination for the phosphorylated peptide library, suggesting that these fragments included a functional phosphopeptide binding domain.

### Identification of Polo-Box Domain as a Phosphopeptide Recognition Module

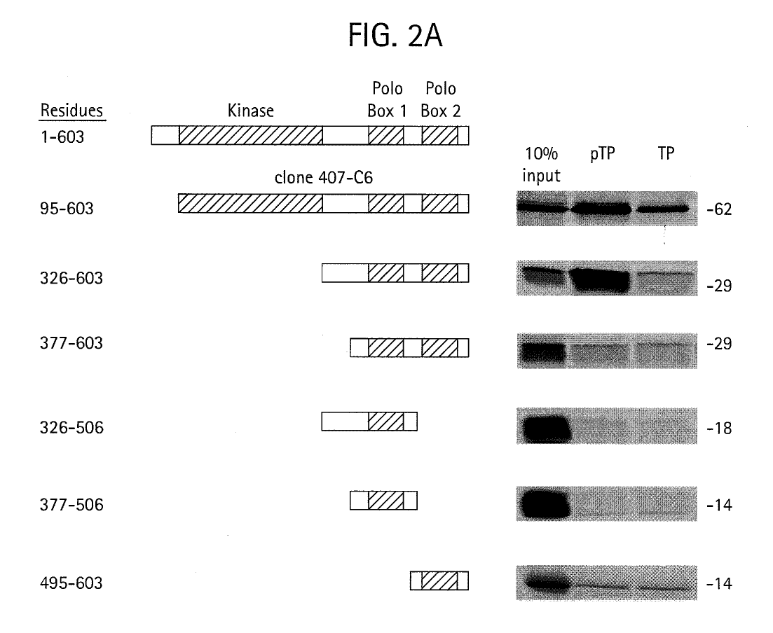

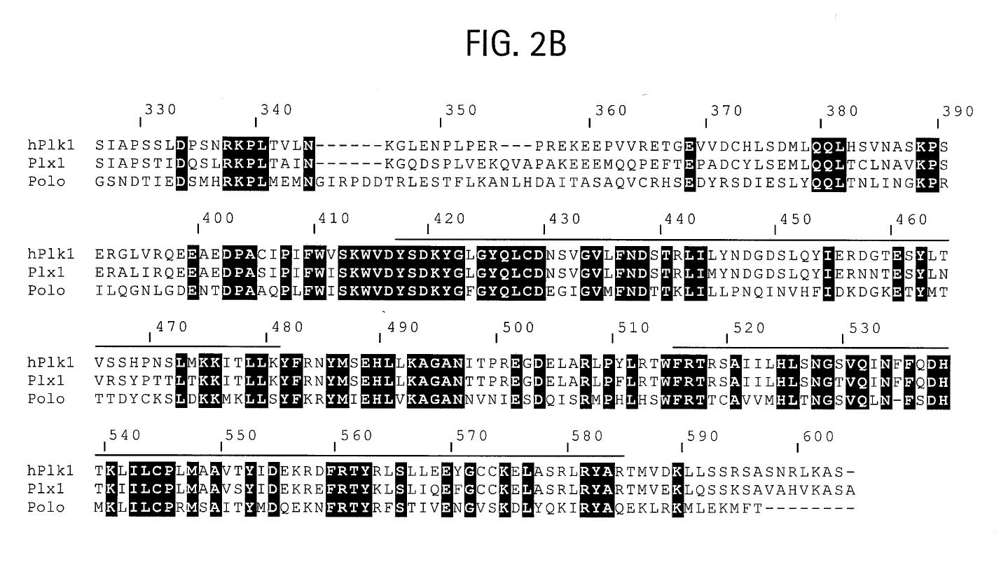

A hallmark feature of the Polo kinase family is the presence of a highly conserved C-terminal region downstream from a conserved amino-terminal kinase domain (FIGS. 2A and B). This region includes two blocks of strong homology, termed Polo Boxes. To define the limiting fragment of Plk-1 responsible for phosphospecific binding, we generated a series of deletion constructs based on an alignment of the C-terminal regions of human Plk-1, Xenopus Plx-1 and Drosophila Polo (FIG. 2B), and analyzed these deletion fragments for phosphopeptide-specific binding. As shown in FIG. 2A, a construct that began immediately after the kinase domain and extended to the last residue of the protein (residues 326-603) demonstrated strong and specific binding to the phosphothreonine-proline peptide library compared with the non-phosphorylated control. Notably, this construct was superior to the parent clone 407-C6 in discriminating for phosphopeptides. Neither of the individual Polo Boxes alone (denoted PB1 and PB2), nor a construct containing both Polo Boxes but lacking the linker region between the kinase domain and PB1, was capable of phosphopeptide binding (FIG. 2A). Furthermore, a construct that included the linker region and PB1 but not PB2 was also unable to bind phosphopeptides. Thus, it appears that the linker region together with both Polo-boxes functions together as a single phosphopeptide-binding module, and we therefore propose that this segment be called the Polo-box Domain (PBD). Intriguingly, this region encompassing both Polo-boxes has been previously shown to regulate the localization of Plk-1 to centrosomes and kinetochores during prophase and to the midbody during late stages of mitosis. Significantly, neither Polo-box alone was sufficient for this localization function, though mutations within PB1 were sufficient to disrupt it.

### The Plk-1 Polo-Box Domain Consensus Motif

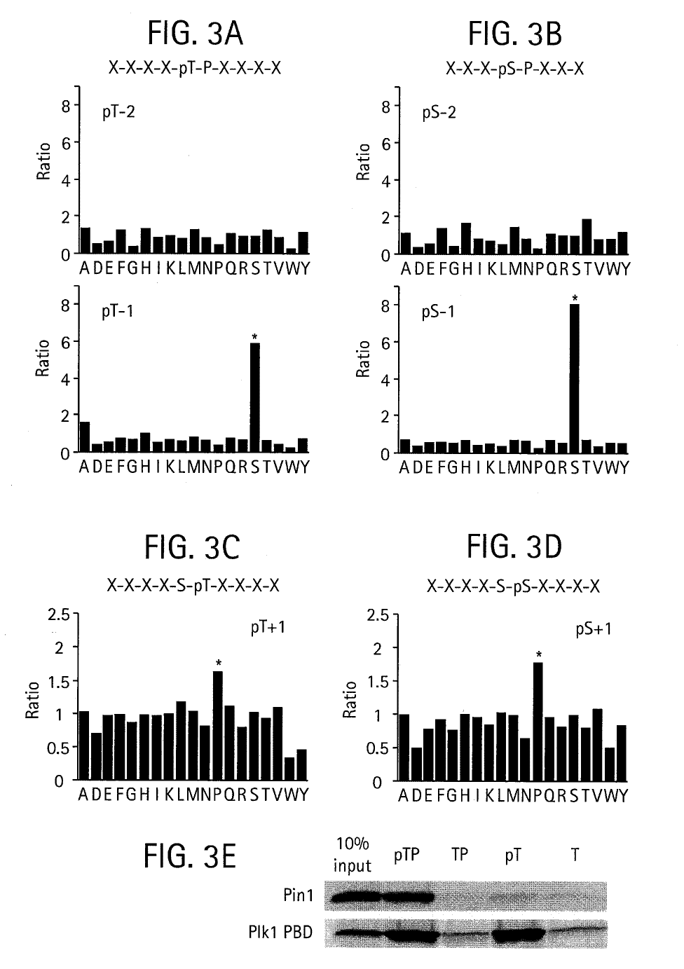

A central feature of our screen for phosphopeptide-binding domains is that any pSer/Thr-binding domain identified through interaction with phosphopeptide library-immobilized beads is amenable to subsequent determination of its optimal binding motif using a standard “forward” peptide library screening approach. A GST fusion protein of the Plk-1 PBD was therefore expressed in bacteria, immobilized on glutathione beads, and incubated with degenerate phosphopeptide libraries oriented on a fixed pThr-Pro (FIG. 3A) or pSer-Pro motif (FIG. 3B). Following extensive washing, the PBD-bound peptides were eluted and sequenced, and the amount of each amino acid in every degenerate position was compared to that present in the starting library mixture to derive amino acid selectivity ratios. Surprisingly, the Plk-1 PBD displayed an extraordinarily strong and novel selection for Ser in the pThr-1 position when the pThr-Pro library was used. Extremely strong selection for Ser was also observed in the −1 position when the PBD was assayed using the fixed pSer-Pro library. Binding of the PBD to a phosphoserine-containing peptide library is noteworthy in itself, since at least one other family of phosphopeptide-binding modules, FHA domains, appear to bind only to phosphothreonine-containing motifs. The relative selection values observed for Ser in either the pThr-1 or pSer-1 position, 5.9 and 8.1 respectively, are among the largest we have observed for any domain whose specificity has been previously determined by peptide library screening.

Since the Plk-1 PBD was isolated in a screen for domains that bind to pThr-Pro motifs, it was important to determine the relative importance of Pro in the pThr+1 position for PBD recognition. To accomplish this, peptide library screens were performed with libraries containing a fixed pThr residue, a fixed pSer residue, fixed Ser-pThr residues, or fixed Ser-pSer residues (Table 1, FIGS. 3C, and 3D). Little selection was observed for proline in the pThr/pSer+1 position when serine was not fixed in the pThr/pSer-1 position (Table 1). Inclusion of serine at this position in a Ser-pThr oriented library, however, unmasked a moderate selection (1.7) for proline at pThr+1 (FIG. 3C and Table 1). Proline selection (1.8) was also uncovered at this position when a Ser-pSer oriented library was used (FIG. 3D and Table 1). Notably, synergistic selection between serine and proline was also observed in reverse such that inclusion of a fixed Pro residue in the peptide libraries led to a higher selection for serine (Table 1).

Table 1, below, summarizes the results obtained from phosphopeptide motif selection screening (SEQ ID NO: 103).

TABLE 1
pT and pS Peptide Motif Selection by Plk-1 Polo Box Domain

| −3 | −2 | −1 | | +1 |
|---|---|---|---|---|
| M (1.3) | A (1.4) | S (5.9) | pT | P |
| Y (1.3) | H (1.4) | A (1.6) | | |
| H (1.3) | M (1.4) | | | |
| F (1.2) | T (1.3) | | | |
| K (1.2) | F (1.3) | | | |
| I (1.4) | A (1.5) | S (3.7) | pT | X |
| K (1.4) | Q (1.3) | A (1.6) | | |
| | T (1.2) | G (1.3) | | |
| M (1.5) | Q (1.5) | S | pT | P (1.6) |
| F (1.4) | A (1.5) | | | M (1.3) |
| L (1.2) | H (1.5) | | | |
| | M (1.4) | | | |
| | F (1.3) | | | |
| | T (1.2) | | | |
| M (1.7) | T (1.9) | S (8.1) | pS | P |
| Y (1.5) | H (1.7) | | | |
| H (1.4) | M (1.5) | | | |
| F (1.3) | F (1.4) | | | |
| K (1.2) | | | | |
| F (1.4) | T (1.9) | S (6.0) | pS | X |
| M (1.3) | H (1.4) | | | |
| Y (1.3) | M (1.3) | | | |
| | A (1.3) | | | |
| M (1.6) | M (1.6) | S | pS | P (1.8) |
| F (1.3) | Q (1.5) | | | M (1.3) |
| Y (1.3) | H (1.5) | | | |
| L (1.2) | A (1.3) | | | |
| | T (1.3) | | | |

A GST fusion of the Plk-1 Polo Box Domain was screened for binding to six phosphopeptide libraries, which contained the sequences MAXXXXpTPXXXXAKKK SEQ ID NO: 46, MAXXXXpTXXXXAKKK SEQ ID NO: 47, MAXXXXSpTXXXXAKKK SEQ ID NO: 48, MAXXXpSPXXXAKKK SEQ ID NO: 49, MAXXXXpSXXXXAKKK SEQ ID NO: 50, and MAXXXXSpTXXXXAKKK SEQ ID NO: 48, where X indicates all amino acids except Cys. Residues showing strong enrichment are underlined. Selection for Pro (1.4) was observed in the −4 position in the X₄SpTX₄ and X₄SpSX₄ screens. Slight selection for aliphatic and aromatic residues was observed in the +2 position in most screens. Little or no selection was observed in the −5, +3, +4, or +5 positions in any of the screens.

These results suggested that the presence of Pro in the pThr/pSer+1 position, while helpful, was not absolutely required for binding. In agreement with this, the Plk-1 PBD bound in a phospho-specific manner to bead-immobilized peptide libraries containing either a fixed pThr-Pro dipeptide or an isolated pThr alone (FIG. 3E). In contrast, the other protein isolated in our screen, full-length Pin1, bound only to the pThr-Pro peptide library beads.

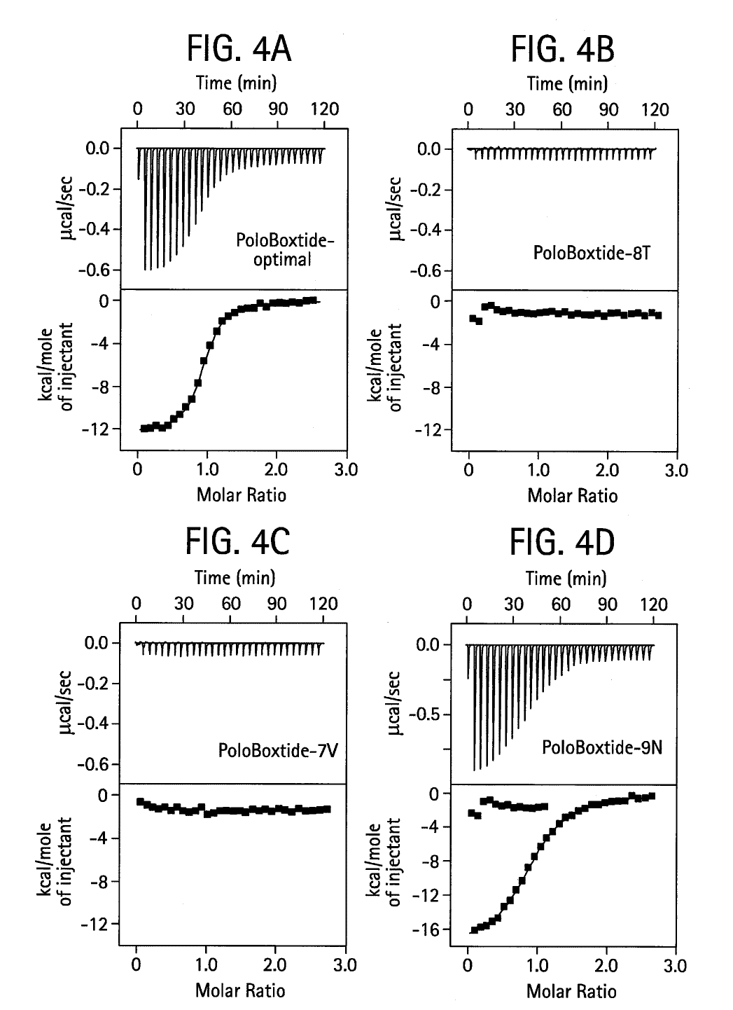

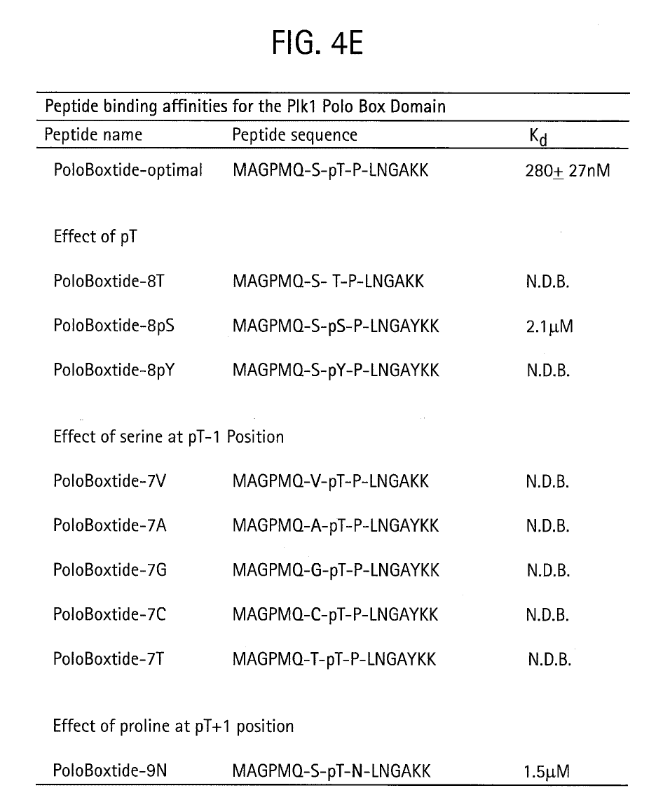

To verify the results of oriented peptide library screening, binding of individual phosphopeptides to the Plk-1 PBD was measured by isothermal titration calorimetry (FIGS. 4A and 4B). The optimal phosphopeptide ligand (PoloBoxtide-optimal), containing the core sequence Met-Gln-Ser-phoshoThr-Pro-Leu (SEQ ID NO: 51) derived from peptide library screening, bound tightly to the Plk-1 PBD with a dissociation constant of 280 nM. Furthermore, it formed a 1:1 protein/peptide complex, indicating that separate phosphopeptides were not interacting simultaneously with each of the two polo boxes within the PBD. Substitution of threonine for phosphothreonine (PoloBoxtide 8T) resulted in complete loss of binding, reiterating the absolute dependence of interaction on the presence of a phosphate group. Substitution of phosphoserine for phosphothreonine within the optimal PBD motif maintained peptide binding to the Plk-1 PBD in agreement with the peptide library screening results, albeit with a seven-fold drop in affinity. In contrast, substitution of phosphotyrosine for phosphothreonine completely abrogated binding, demonstrating conclusively that the Plk-1 PBD is a pThr/pSer-specific binding domain. The extraordinarily strong selection observed for Ser in the pThr/pSer-1 position within the Plk-1 PBD binding motif was confirmed using a series of mutant peptides. When this Ser was replaced with either of the sterically small amino acids Ala or Gly, with the hydroxyl containing amino acid Thr, or with the homologous amino acid Cys, no peptide binding was detectable. Moderate selection for Pro in the pThr/pSer+1 position was verified by a greater than five-fold increase in K_{d} when another β-turn forming residue, Asn, was substituted for Pro in this position. Based on the oriented peptide library screening data (FIG. 3, Table 1) and these ITC results, we therefore propose that the core consensus motif recognized by the Plk-1 PBD is S-[pT/pS]-(P/X) (SEQ ID NO: 52).

### Physiological Substrates of PBD

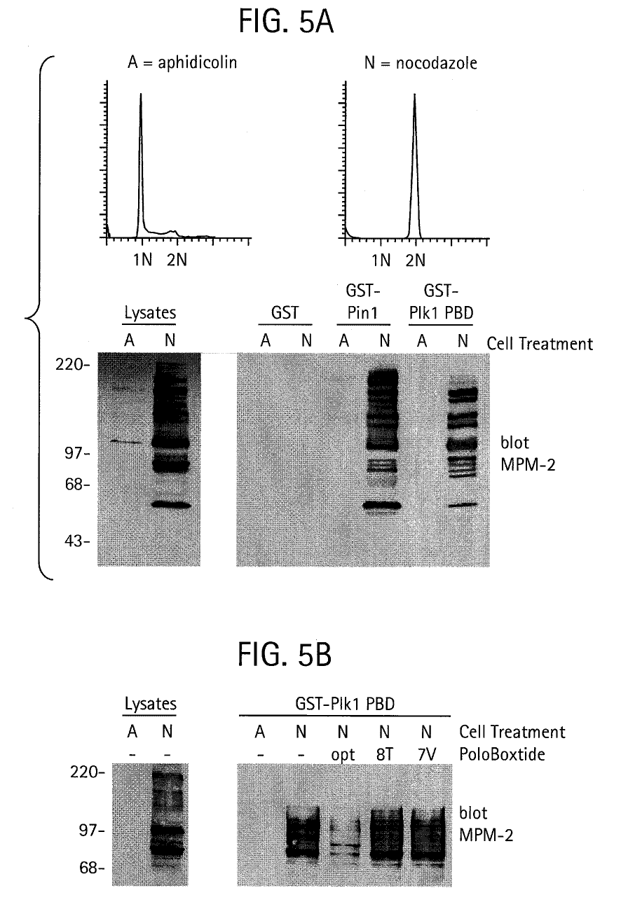

The monoclonal antibody MPM-2 (Mitotic Phosphoprotein Monoclonal-2), originally raised against mitotic HeLa cell extracts, recognizes a conserved pSer/pThr-Pro epitope present on ˜50 phosphoproteins that are localized to various mitotic structures. The initial screen from which the Plk-1 PBD was identified used a peptide library that was partially biased to resemble the MPM-2 epitope. A number of important mitotic regulators that are recognized by this antibody, including Cdc25, Weel, Mytl, Topoisomerase II alpha and inner centromere proteins (INCENP), contain one or more exact matches of the S-[pS/pT]-P PBD-binding motif We therefore investigated whether the Plk-1 PBD bound to MPM-2 reactive proteins. HeLa cells were treated with aphidocolin to induce a G1/S arrest or with nocodazole to induce a G2/M arrest and cell lysates were analyzed by immunoblotting (FIG. 5A). As expected, the number of MPM-2 reactive proteins was greatly enhanced in the mitotically-arrested cells. Many of these MPM-2 reactive mitotic phosphoproteins were specifically bound by the Plk-1 PBD, suggesting that phosphorylation of these proteins by proline-directed mitotic kinases generated a PBD-binding site. Furthermore, the Plk-1 PBD bound to a different and somewhat smaller subset of MPM-2 epitope-containing proteins than those that bound to Pin1 (FIG. 5A), which was expected given that the MPM-2 epitope motif more closely resembles the optimal consensus motif for Pin1 than that of the Plk-1 PBD.

To determine whether the Plk-1 PBD associates with MPM-2 epitopes through its phosphopeptide binding pocket, peptide competition assays were performed. Pre-incubation of the Plk-1 PBD with its optimal phosphopeptide ligand dramatically inhibited the binding of MPM-2 epitopes (FIG. 5B, ‘opt’). In contrast, the non-phosphorylated analogue (‘8T’) or a peptide with Val substituted for Ser in the pT-1 position (‘7V’) had no effect.

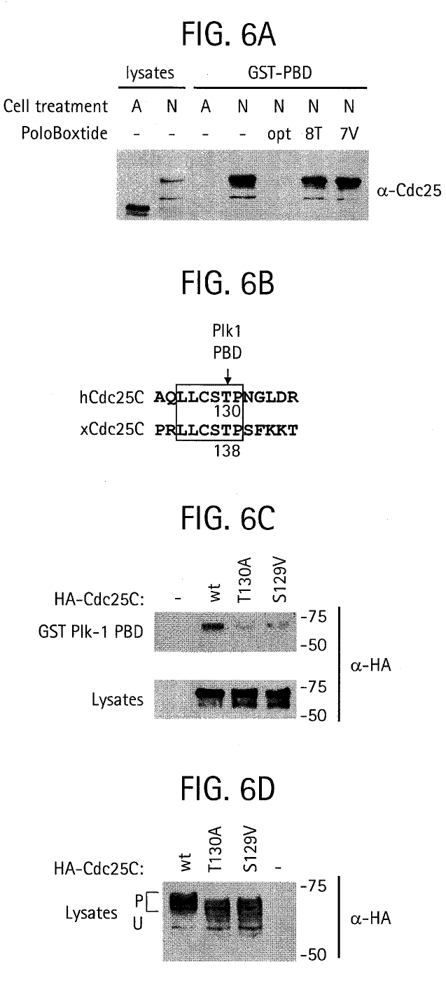

One particular MPM-2 antigen that is also known to be phosphorylated and regulated by Plk-1 and its Xenopus homologue is the cell-cycle regulated protein phosphatase Cdc25. We therefore investigated whether Cdc25C associated with the Plk-1 PBD in a cell-cycle-regulated and phospho-specific manner. During mitosis, Cdc25C undergoes a dramatic reduction in gel mobility due to extensive phosphorylation at its N-terminus. The Plk-1 PBD was found to interact only with this mitotically up-shifted form of Cdc25C (FIG. 6A). Pre-incubation of the Plk-1 PBD with its optimal phosphopeptide ligand, but not with the 8T or 7V mutant peptides, completely prevented this association, demonstrating that it was mediated through the phosphopeptide binding pocket of Plk-1. During mitosis, Cdc25C is known to be phosphorylated on five conserved Ser/Thr-Pro sites within its N-terminus. One of these sites, Thr₁₃₀ (corresponding to Thr₁₃₈ in Xenopus Cdc25C) contains a conserved Plk-1 PBD consensus motif (FIG. 6B). To investigate whether this site was important for the Cdc25C-Plk-1 interaction, HeLa cells were transfected with HA-tagged wild-type Cdc25C, or with Thr₁₃₀Ala or Ser₁₂₉Val point mutants of Cdc25C expected to disrupt the PBD-binding motif. Following mitotic arrest with nocodazole, the Plk-1 PBD bound strongly only to the wild-type protein, but only very weakly to either of the point mutants, indicating direct interaction between the Plk-1 PBD phosphopeptide-binding pocket and a mitotically-phosphorylated PBD consensus motif in Cdc25C (FIG. 6C). Furthermore, both of these point mutants had a decreased electrophoresis mobility shift when analyzed on lower percentage gels (FIG. 6D), suggesting that mutations which impair Plk-1 PBD binding result in incomplete Cdc25C phosphorylation in vivo.

### Centrosomal Localization of the Plk-1 PBD Occurs Through its Phosphopeptide-Binding Pocket.

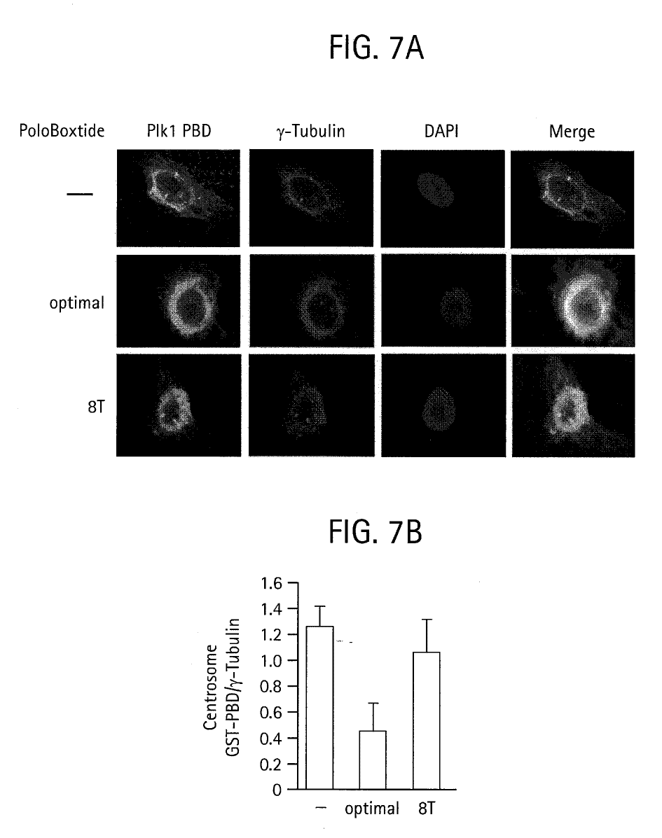

Plk-1 localizes to centrosomes and kinetochores in prophase and to the spindle mudstone during late stages of mitosis. Centrosomal localization has been shown to require both the PB1 and PB2 regions, but not kinase activity, since localization is maintained when Lys₈₂, which is mediates phosphate transfer, is mutated to Met. To investigate whether the phosphopeptide binding function of the Plk-1 PBD was critical for its centrosomal localization, U2OS cells were mitotically arrested with nocodazole, permeablized with Streptolysin-O, and incubated with GST-Plk-1 PBD in the absence or presence of peptide competitors. The Plk-1 PBD was observed to localize to the centrosomes of late prophase-arrested cells (FIG. 7A), as verified by co-staining with an anti-γ-tubulin antibody.

This centrosomal localization was significantly disrupted in the presence of an optimal Plk-1 PBD phosphopeptide but was unaffected when the assay was performed using the same concentration of the non-phosphorylated peptide analogue (FIGS. 7A and 7B). This observation, together with published data showing that the C-terminus of Polo-like kinases is essential for their function in vivo, strongly suggests that intracellular targeting of Plk-1 to critical substrates is mediated through interaction of the PBD phosphopeptide pocket with phosphorylated motifs in mitotic structures.

### The Plk-1 PBD and Regulation of Mitotic Progression by Cyclin-Dependent Kinase Priming

Our identification of the Plk-1 PBD as a novel phosphoserine/threonine-binding domain adds another member to the growing superfamily of pSer/Thr-binding modules and demonstrates the general utility of our phospho-motif-based affinity screen for discovering and functionally characterizing novel signaling domains that function downstream of protein kinases. This screening technique can be used to identify binding modules interacting with substrates of any kinase whose phosphorylation motif is known. Other techniques that identify protein-protein and protein-peptide interactions, such as yeast 2-hybrid and phage display approaches cannot be used in screens for phospho-binding domains since reliable and constitutive phosphorylation of a diverse collection of bait sequences is required. A further strength of our technique is that any domain isolated through screening with bead-immobilized peptide libraries yields an optimal consensus binding motif when the domain is subsequently analyzed by traditional peptide library screening. This allows the motif for the pSer/Thr-binding domain to be combined with that of the potential phosphorylating kinase(s) in database searching and protein sequence analysis and should facilitate the proteome-wide prediction of ligands within a common signaling pathway.

The C-terminal region of Polo-like kinases has long been recognized as essential for their in vivo function in mitosis and cytokinesis, but its structural mechanism has remained mysterious. Mutations within this region of Plk-1 and its S. cereviseae homologue, CdcS, abolish their ability to rescue a temperature-sensitive mutant of cdc5 despite the presence of a fully functional kinase domain. When expressed alone, the C-terminal domain of Polo-like kinases localizes to centrosomes and the spindle midzone similar to the full-length kinase, and its overexpression causes mitotic and cytokinetic arrest.

We have shown that the C-terminal domain of Plk-1 is a phosphoserine/threonine-binding module whose phospho-binding pocket binds to known Polo substrates and mediates localization to subcellular sites where endogenous Polo kinases are found. In the basal state the PBD binds to the kinase domain, inhibiting its phosphotransferase activity. In addition to overcoming this inhibition, maximal activation of the kinase domain also requires phosphorylation in its activation loop by upstream kinases such as xPlkk1/SLK. This requirement for both priming phosphorylation of substrates and activation loop phosphorylation provides a molecular switch that regulates Plk-1 kinase function at discrete stages of the cell cycle. In addition, it provides a potential means for mitotic checkpoint control, since neither phosphorylation of the activation loop nor substrate priming phosphorylation alone would be sufficient for proper activation of Polo kinases in vivo.

A number of striking parallels between the PBD of Plk-1, SH2 domains in Src family kinases, and FHA domains in the Rad53/Chk2 family of checkpoint kinases are apparent. Like the Plk-1 PBD, SH2 domains of Src-family kinases both inhibit kinase activity in the inactive state and facilitate substrate targeting when Src kinases have been activated by phosphorylation on their activation loops. In Src kinases, the mechanism of inhibition involves intramolecular binding of the SH2 domain to a pTyr motif at the end of the kinase domain. It remains unknown whether Polo kinase family inhibition by the PBD involves a similar interaction with internal pSer/pThr sites, or whether an alternative PBD surface is involved. Members of the Chk2 kinase family contain one or more pThr-binding FHA domains in addition to the kinase module. The FHA domain(s) are critical for proper Chk2 function in response to DNA damage and for the phospho-dependent targeting of Chk2 into larger multimolecular complexes where activation occurs.

We found the optimal motif for Plk-1 PBD binding to be S-[pS/pT]-P/X. Differences in PBD selectivity for amino acids flanking the pSer/Thr position are likely to be biologically important for the interaction of Polo kinases with their substrates in vivo. The primary role of the +1 Pro may be to link phospho-dependent PBD binding to activation of cyclin-dependent kinases that phosphorylate the motif, providing a means to temporally and spatially regulate the action of Polo-like kinases during mitosis. The absolute requirement for Ser in the −1 position provides strong discrimination for Plk-1 binding to only a limited subset of mitotic kinase substrates. In addition, we found that the motif recognized by the Plk-1 PBD partially overlaps with the proline-directed sequence motif recognized by the monoclonal antibody MPM-2 which reacts against a large number of mitotically phosphorylated proteins, and we demonstrated a direct interaction between the PBD phosphobinding pocket and MPM-2 reactive proteins in pull-down experiments with mitotic cell extracts. This finding provides an elegant explanation for the progressive accumulation of MPM-2 immuno-reactivity and Polo kinase localization observed at maturing centrosomes, and suggests that generation of MPM-2 epitopes by Cdks and other mitotic kinases triggers PBD-mediated recruitment of Polo kinases to specific mitotic structures.

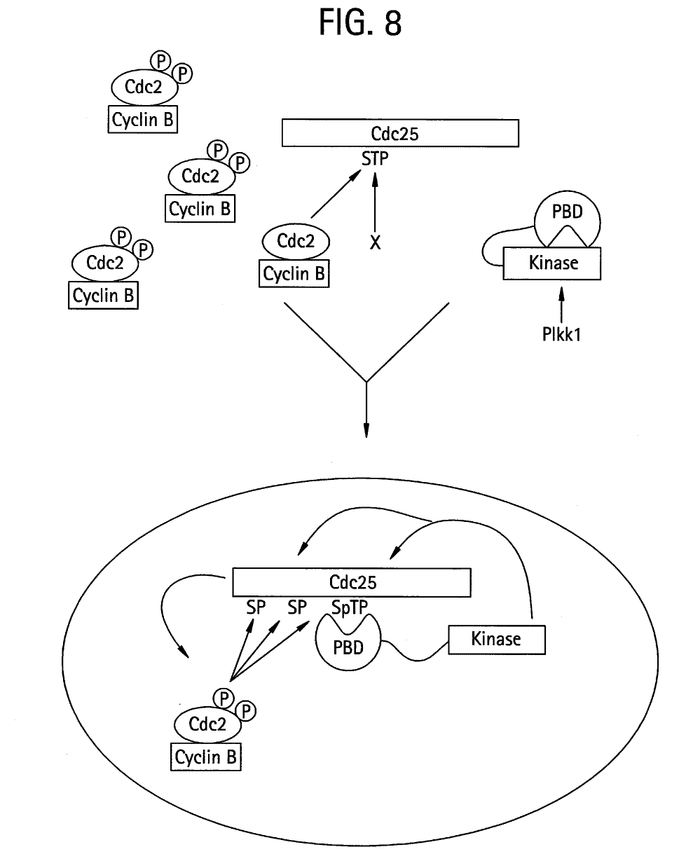

Both Cdks and Polo kinases have been implicated in activating the phosphatase Cdc25, leading to desphosphorylation and activation of Cdc2/Cyclin B and progression through mitosis. The relative roles of Cdks and Polo kinases in Cdc25 activation, however, remains controversial. Our finding that the Plk-1 PBD binds to one or more critical Cdk sites on Cdc25C suggests a molecular rationale for 2-step activation of Cdc25 that has been postulated to drive auto-amplification of Cdc2/CyclinB activity. In prophase, low levels of Cdc2/CyclinB activity are insufficient to fully activate Cdc25, but provide priming phosphorylation of Cdc25 for interaction with the PBD. Subsequent activation of Polo kinases later in mitosis by activation loop kinases such as Plkkl/SLK leads to an initial wave of Cdc25 activation, which generates more Cdc2/Cyclin B activity, primes additional Cdc25 molecules for activation by Polo-like kinases, and results in a positive feedback loop for the production of additional Cdc2/Cyclin B activity (FIG. 8). This model is able to explain the result of Toyoshima-Morimoto et al. (EMBO Rep., 3:341-348, 2002) that maximal intracellular targeting and activation of Cdc25, even in the presence of constitutively active Plk-1, still requires the co-expression of Cyclin B1.

Increased levels of Plk expression have been detected in a variety of human tumors and tumor cell lines, and high levels of expression correlate with poor prognosis. The PBD would be an attractive target for the design of anti-proliferative chemotherapeutics since its compact tripeptide binding motif may be particularly amenable to the design of small molecule peptidomimetics.

Optimal phosphopeptide-binding motifs for the PBDs from all members of the human Plk family, Xenopus Plx1 and Saccharomyces cerevesiae Cdc5p were determined by oriented peptide library screening as described above. Since we initially isolated the Plk1 PBD in a search for domains that recognize a pThr-Pro-containing motif, primary screens were performed using peptide libraries containing a fixed pThr-Pro core flanked on both sides by four degenerate positions. As seen in Tables 2 and 3, the five PBD's examined each selected for distinct but largely overlapping motifs: Plk1 (SEQ ID NO: 104), Plk2 (SEQ ID NO: 105), Plk3 (SEQ ID NO: 106), Plx 1 (SEQ ID NO: 107), and CdcS (SEQ ID NO: 108).

TABLE 2

Phosphothreonine Peptide Motif Selection by
Human Polo Kinase Family PBDs

| | | | | | | |
|---|---|---|---|---|---|---|
| −5 | −4 | −3 | −2 | −1 | +1 | +2 |

Plk1

| | | | | | | | |
|---|---|---|---|---|---|---|---|
| | M (1.5) | M (1.3) | A (1.4) | S (5.9) | pT | P | F (1.2) |
| | F (1.1) | Y (1.3) | H (1.4) | A (1.6) | | | I (1.2) |
| | | H (1.3) | M (1.4) | | | | K (1.2) |
| | | F (1.2) | T (1.3) | | | | |
| | | K (1.2) | F (1.3) | | | | |
| P (1.4) | P (1.5) | M (1.5) | Q (1.5) | S | pT | P (1.6) | L (1.2) |
| F (1.1) | F (1.3) | F (1.4) | A (1.5) | | | M (1.3) | K (1.1) |
| | M (1.3) | L (1.2) | H (1.5) | | | | V (1.1) |
| | L (1.2) | | M (1.4) | | | | |
| | I (1.1) | | F (1.3) | | | | |
| | | | T (1.2) | | | | |

Plk2

| | | | | | | | |
|---|---|---|---|---|---|---|---|
| | F (1.9) | Q (1.9) | T (2.1) | S (7.5) | pT | P | F (1.5) |
| | I (1.6) | M (1.8) | H (2.1) | | | | L (1.5) |
| | M (1.5) | H (1.6) | Q (1.2) | | | | I (1.3) |
| | L (1.4) | F (1.3) | | | | | V (1.1) |
| | P (1.1) | | | | | | |
| P (2.4) | M (1.5) | Q (1.9) | T (2.8) | S | pT | P (1.7) | K (1.5) |
| F (1.4) | F (1.5) | T (1.6) | H (2.0) | | | | L (1.2) |
| I (1.2) | P (1.4) | M (1.6) | Q (1.7) | | | | I (1.1) |
| | L (1.4) | H (1.6) | | | | | |
| | I (1.3) | F (1.2) | | | | | |
| | V (1.2) | | | | | | |

Plk3

| | | | | | | | |
|---|---|---|---|---|---|---|---|
| | I (1.5) | M (1.6) | T (1.6) | SP (3.0) | pT | P | K (1.3) |
| | L (1.4) | L (1.3) | H (1.4) | | | | V (1.2) |
| | V (1.3) | F (1.3) | | | | | F (1.2) |
| | F (1.2) | | | | | | |
| | P (1.2) | | | | | | |
| P (1.2) | L (1.2) | A (1.5) | T (2.6) | S | pT | P (1.6) | K (1.4) |
| | I (1.2) | M (1.2) | H (1.6) | | | D (1.4) | |
| | | F (1.2) | | | | E (1.3) | |
| | | I (1.2) | | | | | |

GST fusions of the Polo-box Domains (PDBs) from hPlk1, hPlk2, and hPlk3 were screened for binding to phosphopeptide libraries containing the sequences MAXXXXpTPXXXXAKKK (SEQ ID NO: 46) and MAXXXXSpTXXXXAKKK (SEQ ID NO: 48), where X indicates all amino acids except Cys. Residues showing strong enrichment are underlined.

TABLE 3

Phosphothreonine Peptide Motif Selection
by Polo Kinase PBD Orthologs

| | | | | | | |
|---|---|---|---|---|---|---|
| −5 | −4 | −3 | −2 | −1 | +1 | +2 |

Plx1

| | | | | | | | |
|---|---|---|---|---|---|---|---|
| | F (2.1) | F (1.5) | T (2.1) | S (7.3) | pT | P | I (1.6) |
| | I (1.6) | L (1.5) | H (1.7) | | | | L (1.5) |
| | L (1.3) | M (1.5) | | | | | V (1.1) |
| | M (1.2) | | | | | | |
| P (1.8) | P (1.6) | F(1.6) | T (3.0) | S | pT | P (1.9) | K (1.4) |
| F (1.4) | F (1.5) | M (1.5) | H (1.6) | | | | I (1.3) |
| | L (1.5) | L(1.4) | Q (1.3) | | | | L (1.2) |
| | I (1.4) | | | | | | |
| | M (1.3) | | | | | | |

Cdc5

| | | | | | | | |
|---|---|---|---|---|---|---|---|
| | M (1.9) | A (2.5) | T (2.4) | S (5.3) | pT | P | X |
| | L (1.5) | M (1.5) | A (1.8) | | | | |
| | I (1.4) | F (1.1) | Q (1.5) | | | | |
| | F (1.2) | | M (1.4) | | | | |
| | | | H (1.4) | | | | |
| P (2.8) | L (2.2) | A (3.4) | A (2.1) | S | pT | P (1.4) | L (1.3) |
| F (1.3) | M (1.7) | V (1.3) | Q (1.7) | | | | I (1.1) |
| | I (1.5) | I (1.2) | T (1.6) | | | | |
| | F (1.5) | | H (1.6) | | | | |
| | V (1.1) | | M (1.3) | | | | |

GST fusions of the Polo-box Domains (PDBs) from Xenopus Plx1 and S. Cerevisiae Cdc5p were screened for binding to phosphopeptide libraries containing the sequences MAXXXXpTPXXXXAKKK (SEQ ID NO: 46) and MAXXXXSpTXXXXAKKK (SEQ ID NO: 48), where X indicates all amino acids except Cys. Residues showing strong enrichment are underlined.

All of the PBDs showed unequivocal selection for Ser in the pThr-1 position with selectivity ratios (i.e. the mol % of Ser in the PBD-bound peptides at the pThr-1 position divided by the mol % of Ser in the starting library mixture at the pThr-1 position) ranging from 3.0 to 7.5. Motif similarity occurs even though these PBDs vary considerably in amino-acid sequence and the respective human Plks perform divergent cellular functions. The PBDs as a group consistently demonstrated moderate selection for Thr, His, Gln, and Met in the pThr-2 position. There was general selection amongst all PBDs for aliphatic and aromatic residues in the pThr-3, pThr-4 and pThr+2 positions, although Cdc5p showed a particularly strong and unique selection for Ala in the pThr-3 position, while Plk2 showed strong and unique selection for Gln at this position. All PBDs except Cdc5p also selected for Pro in the pThr-4 position and Lys in the pThr+2 position

Based on these data, secondary peptide libraries containing a fixed Ser-pThr core were used to further refine the motifs and investigate the relative importance of Pro in the pThr+1 position. These screens revealed modest selection for Pro at pThr+1 for all PBDs, with selectivity ratios ranging from 1.4 to 1.9 (Tables 2 and 3). Selection at other motif positions for each PBD was consistent with those obtained using the pThr-Pro library, though we were now able to observe significant and conserved selection for Pro and Phe in the pThr-5 position. (pT-5 was degenerate in the Ser-pThr library, but was a fixed Ala residue in the pThr-Pro-oriented library.) Thus, it appears that the PBDs of all Plks investigated, including all conventional human Plk homologues, select a similar motif that can be most generally represented by the consensus sequence: [Pro/Phe]-[φ/Pro]-[φ/Ala_{Cdc5p}/Gln_{Plk2}]-[Thr/Gln/His/Met]-Ser-[pThr/pSer]-[Pro/X] SEQ ID NO: 2, where φ represents hydrophobic amino acids.

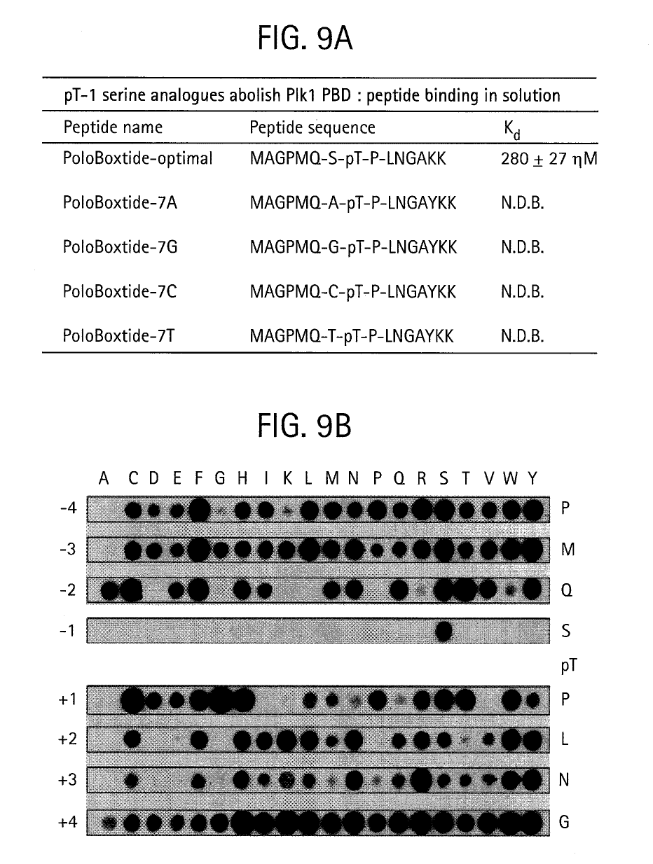

The striking selection observed for Ser in the pThr-1 position in all PBDs was examined in detail for the human Plk1 PBD, which binds to its optimal motif, Pro-Met-Gln-Ser-pThr-Pro-Leu (SEQ ID NO:6) (Table 2), with a K_{d} of 280 nM (FIG. 9A).

A variety of small side-chain amino-acids were therefore substituted in the pThr-1 position, and peptide binding to the Plk1 PBD measured using isothermal titration calorimetry (ITC) (FIG. 9A). Surprisingly, replacement of Ser with Gly, Ala, the hydroxyl-containing amino-acid Thr, or the Ser isostere Cys, completely abrogated Plk1 PBD-phosphopeptide binding. We had previously observed that replacement of Ser at the pThr-1 position with Val, the amino-acid showing the lowest selection in this position, was sufficient to eliminate peptide binding (Elia et al., Science 299:1228-1231, 2003). Nevertheless, the finding that replacement of Ser with a variety of chemically similar amino acids also completely disrupted the interaction between the PBD and free phosphopeptides in solution was unexpected.

To extend this analysis, each amino acid in the eight positions flanking the phosphothreonine within the optimal Plk1 PBD binding motif was substituted with each of the remaining nineteen naturally occurring amino acids using a solid phase array of immobilized phosphopeptides (FIG. 9B). This conclusively demonstrated that only Ser was tolerated in the pThr-1 position (FIG. 9B). Selectivities at other positions were generally consistent with the results of oriented peptide library screening. Cys and Gly, however, were selected at the pThr+1 position at least as strongly as Pro in the immobilized phosphopeptide assay. Cys is routinely omitted during construction of oriented peptide libraries to minimize cross-linking and oxidation effects. Higher relative selection for Gly in the context of immobilized peptides than in solution phase peptide library assays may be due, in part, to the greater entropic penalties associated with ordering Gly residues compared with Pro residues when both ends of a peptide are free. Alternatively, these subtle differences may reflect the fact that the peptide filter assay examines individual point mutations in the context of a single amino-acid sequence, while oriented peptide library screening samples an entire ensemble of sequence motifs simultaneously. Regardless, Pro probably represents the most ‘physiological’ amino acid in the pThr+1 position, since the phosphorylation event necessary for PBD binding is likely to be catalyzed primarily by Pro-directed kinases such as Cdks and MAP kinases.

### Overall Structure of the Plk1 PBD

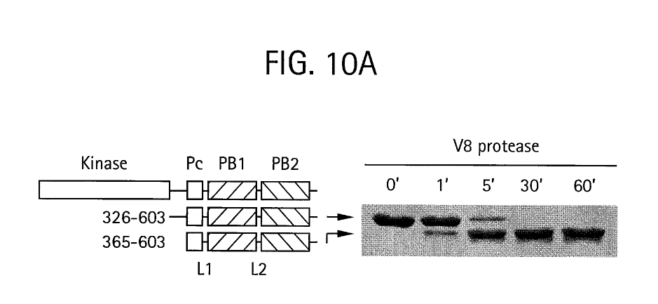

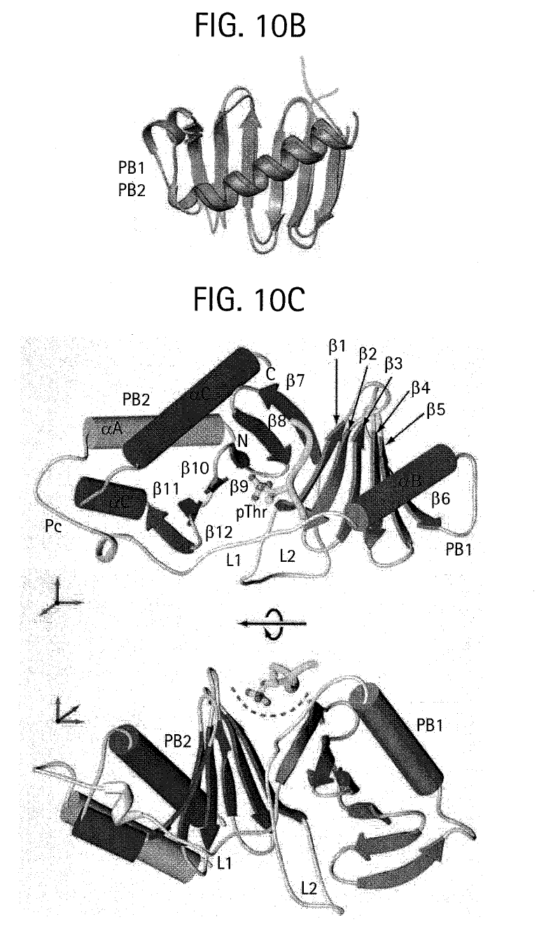

The boundaries of the minimal PBD within the C-terminal regions of both Plk1 and Cdc5p were determined using limited proteolysis and mass-spectrometry. Studies using V8 protease (FIG. 10A) and trypsin (data not shown) indicated that only the last 45 residues of the linker between the kinase domain and the first Polo-box were structured as part of the PBD (FIG. 10A). Similar results were obtained using the C-terminal segment of Cdc5p (data not shown). We refer to the beginning of this additional region as the Polo-cap (Pc). For both Plk1 and Cdc5p, we found no significant difference in the phosphopeptide-binding affinities of fragments encompassing the entire C-terminal regions or the proteolytically-defined PBDs, indicating that the first ˜40 amino acids between the kinase and the Pc plays no major role in peptide binding. Shorter fragments of both Plk1 and Cdc5p encompassing just the Polo boxes, but lacking the Pc, were insoluble in E. coli, indicating a clear structural role for the Pc in both proteins, despite the absence of any extensive sequence homology between the two proteins in this region.

The X-ray structure of a recombinant form of the proteolytically-defined Plk1 PBD (residues 367-603) in complex with its ‘optimal’ phosphopeptide was solved by multiwavelength anomalous diffraction (MAD) using Se-Met-containing protein, and refined against native data extending to 1.9 Å resolution (Table 4).

TABLE 4
Crystallographic analysis
Data Collection

| Dataset (λÅ) | Native (0.98) | Se (0.97838) | Se (0.97887) | Se (0.95) |
|---|---|---|---|---|
| | 14.1 - SRS | | 14.2 - SRS | |
| d (Å) | 20.0-1.9 | 20.0-3.5 | 20.0-3.5 | 20.0-3.5 |
| Completeness (%) | 97.7 | 99.9 | 99.0 | 99.2 |
| Redundancy¹ | 3.6 | 3.7³ | ~1.9³ | ~1.9³ |
| R [symbol] (%)² | 5.3 | 5.4³ | 5.2³ | 4.9³ |

Phasing analysis

| | | | | | | | | |
|---|---|---|---|---|---|---|---|---|
| Resol bin (Å) | 20-11.2 | 11.2-7.5 | 7.5-6.0 | 6.0-5.2 | 5.2-4.6 | 4.6-4.2 | 4.2-3.9 | 3.9-3.6 |
| FOM | 0.79 | 0.83 | 0.79 | 0.70 | 0.59 | 0.53 | 0.48 | 0.44 |
| Mean FOM | 0.60 | | | | | | | |

Refinement

| R [symbol] (%)⁴ | R [symbol] (%)⁵ | [symbol] (Å) | rmx [symbol] (deg.) |
|---|---|---|---|
| 24.0 | 26.8 | 0.007 | 1.2 |

¹N [symbol] /N [symbol]
²R [symbol] − Sⱼ|<I> − Iⱼ|/S<I> where Iⱼ is the intensity of the jth reflection and <I> is the average intensity.
³Calculated with Bijvoets seperated
⁴R [symbol] = S [symbol] |Fᵣₑₛ − F_{calc}|/S [symbol] F [symbol]
⁵R [symbol] = as for R [symbol] but calculated on 5% of the data excluded from the refinement calculation. [symbol]
indicates data missing or illegible when filed

The structure (FIG. 10B) shows that the PBD contains two β₆α motifs that comprise the two Polo-box regions (PB1 & 2) identified by sequence profiling. The atomic structural coordinates of this structure are provided in Table 5. In spite of the fact that the amino-acid sequences of the two Polo-boxes within any one Plk exhibit only ˜20-25% sequence identity, the structures of the two motifs are quite similar (root mean square (rms) deviation of 77 Cα atoms of 1.6 Å; FIG. 10B). The two Polo-boxes pack together to form a 12-stranded β-sandwich flanked by three α-helical segments (FIG. 10C). Although motifs resembling the Polo-box structure are represented in the Protein Databank, the overall domain structure represents a new protein fold.

The Pc consists of an α-helical segment αA, loop, and short 3₁₀ helix which connects to the N-terminal β-strand of Polo-box 1 (β1) through a ˜10 residue linker region (L1). The Pc wraps around Polo-box 2 like a hook tethering it to Polo-box 1. αA packs against αC from PB2 in an anti-parallel coiled-coil arrangement, while the 3₁₀ helix packs against the shorter αC′. The two Polo-boxes are connected by a second ˜30 residue linker sequence (L2) that is partially conserved. L1 and L2 run in anti-parallel directions between the two Polo-box β-sheets. Thus, the hydrophobic core is formed from direct interactions of highly conserved non-polar residues predominantly located on β1/β2 from PB1 and β6/β7 from PB2, together with an array of interactions with the intercalating linker regions.

### Novel PBD-Phosphopeptide Interactions are Crucial for Specificity

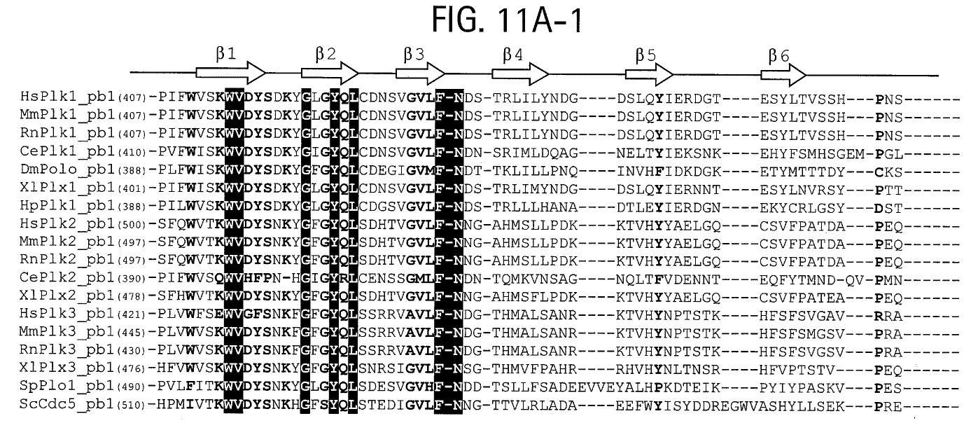

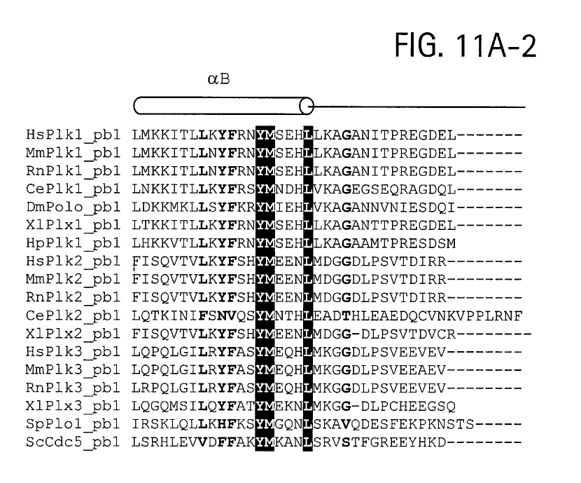

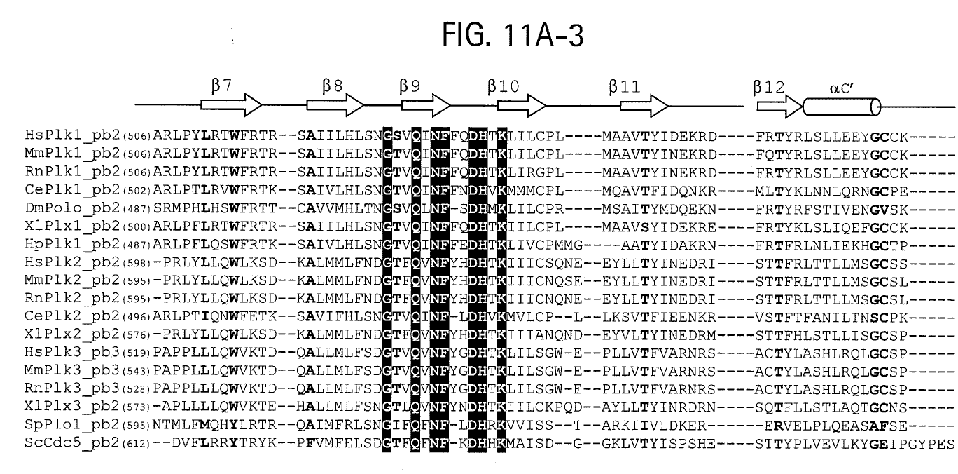

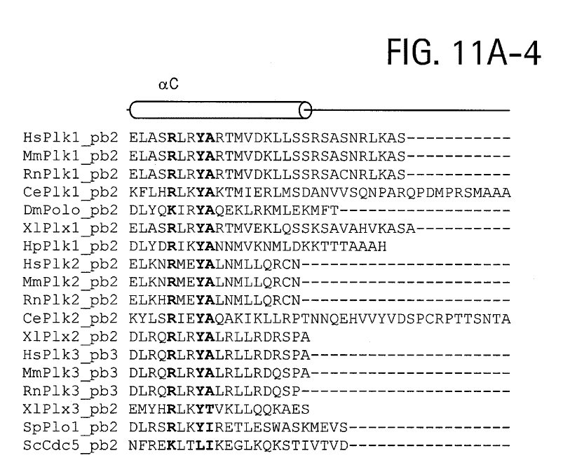

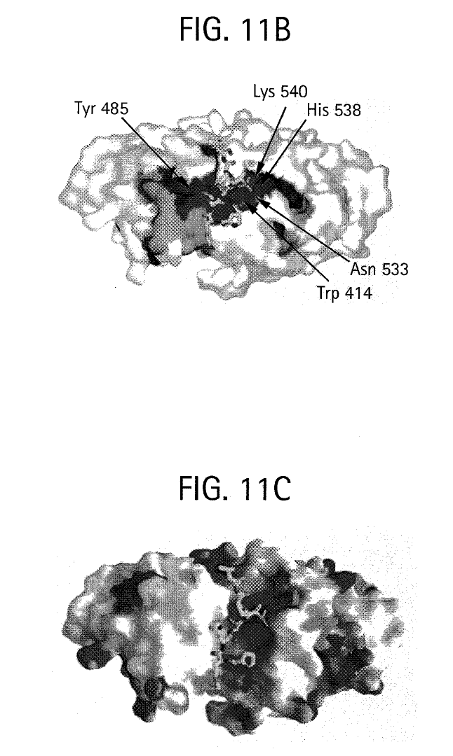

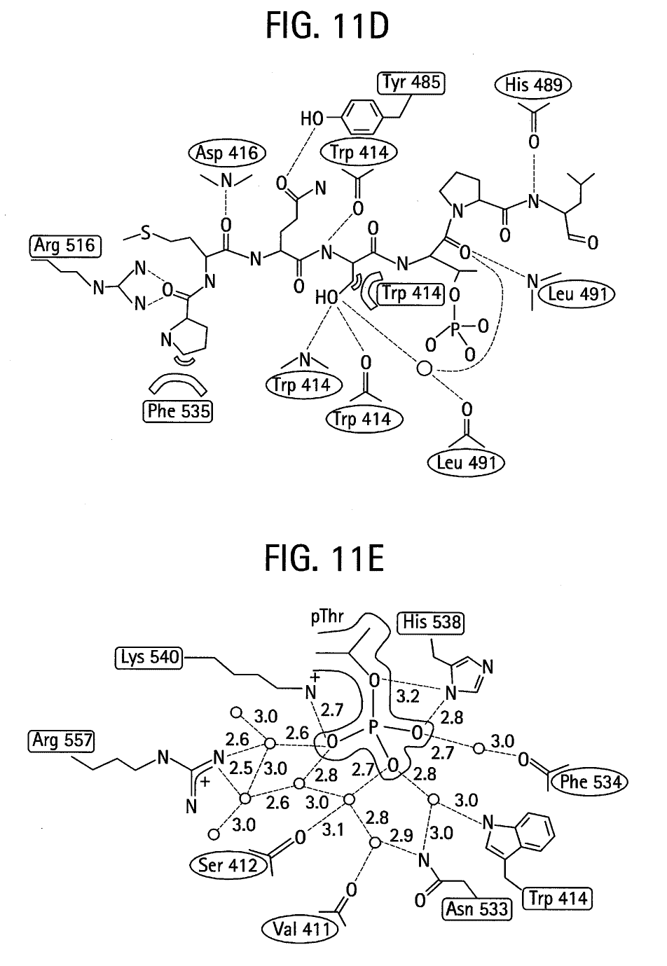

The phosphopeptide binds in a largely extended conformation to a region of positive charge, located at one end of a shallow cleft formed between the two Polo-boxes (FIG. 10). In all, ˜1000 Å² of solvent accessible surface are buried by binding of the seven phosphopeptide residues that are visible in our electron density maps. Binding involves part of an extensive, highly conserved surface that is located exclusively on the peptide-binding face of the PBD (FIG. 11A, 11B). This conserved surface coincides with the only significant region of positive electrostatic potential within the entire PBD (FIG. 11C). Overall, the phosphopeptide interacts predominantly with β1 from PB1, the N-terminal end of L2 and β8 and 9 from PB2. Hydrogen bonding interactions formed with the peptide side- and main-chain atoms alternate to some degree between residues within the two Polo-boxes, forming a zipper-like structure at the edge of the PB1/PB2 interface (FIG. 11D).

PBD binding to the phosphate moiety involves a combination of direct contacts with protein side-chains together with extensive indirect interactions through a well-defined lattice of water molecules, many of which are fully hydrogen-bonded (FIG. 11E). In total, the phosphate group participates in eight hydrogen-bonding interactions explaining the critical dependence on peptide phosphorylation for binding (Elia et al., Science 299:1228-1231, 2003). The only residues that contact the phosphate group directly are His-538 and Lys-540 from PB2, whose side chains form a pincer-like arrangement that chelates the O1, O3, and Oγ phosphate oxygens.

The structural basis for the extraordinarily high selectivity for serine at the pThr-1 position results from a major difference in orientation of the bound phosphopeptide when compared with phosphopeptide complexes of 14-3-3 proteins and FHA domains, the two major classes of pSer/pThr binding proteins (Durocher et al., Mol. Cell. 6:1169-82, 2000; Yaffe et al., Cell 91:961-971, 1997). In these structures, the pThr-1 side-chain is solvent exposed and little selection is observed at this position. In contrast, the peptide orientation in the Plk1 complex is inverted such that the Ser-1 side-chain is directed towards the Plk1 surface (FIG. 11B). In this orientation, it engages in two hydrogen bonding interactions with Trp-414 main-chain atoms, and one with the Leu-491 main-chain carbonyl via a water molecule (FIG. 11C). Significantly, the Ser-1 Cβ atom makes favourable van der Waals interactions with Cδ1 from the Trp-414 indole side-chain. This explains why even a conservative replacement of Ser with Thr at this position abrogates peptide binding (FIG. 9A), presumably due to a steric clash of the threonine γ-methyl substituent with Trp-414.

The critical role of Trp-414 in ligand binding revealed by our crystal structure (FIG. 11D) explains the observation that a W414F mutation eliminates both centrosomal localization of Plk1 and its ability to complement the cdc5-1 is mutation (Lee et al., Proc. Natl. Acad. Sci. USA 95:9301-9306, 1998). Both of these effects are likely to be at least partly attributable to disruption of critical Ser-1 interactions with the PBD. In agreement with this, a mutant PBD containing the W414F substitution is severely compromised in phosphopeptide binding, with an affinity of >100 μM as determined by ITC. Loss of binding is unlikely to result from gross structural perturbation of the Polo-box fold, since the mutant PBD exhibits similar secondary structural content to the wild-type protein as judged from far UV CD spectra (data not shown). Furthermore, Trp-414 in Polo-box 1 is replaced by tyrosine in PB2 of both wild-type S. pombe Plol and S. cerevisiae Cdc5p PBD's, (FIG. 11A), showing that similar substitutions are naturally tolerated in a related structural context.

Consistent with the oriented library selection, the protein-peptide interface is dominated by interactions of the PBD with the pThr and Ser-1 (FIG. 11C, 11D). Although we observed modest selection for Pro at the pThr+1 position, it appears from the structure that it does not contribute greatly to the binding interface, and multiple substitutions at this position are tolerated for peptide binding (FIG. 9B). In the PBD structure, the trans-proline introduces a kink after the Ser-pThr directing the peptide backbone back toward the binding surface, allowing the pThr+2 main chain amino group to contact the PBD. Thus, the +1 Pro likely increases binding affinity by diminishing the entropic penalty for making this favorable backbone contact. This contrasts with structures of pSer-Pro peptide complexes of both the Pin1 WW and the Cdc4 WD40 domains in which the Pro+1 side chain inserts into a hydrophobic pocket and makes coplanar interactions with a buried tryptophan (Leung et al., Nat. Struct. Biol. 9:719-724, 2002; Verdecia et al., Nat Struct Biol 7:639-643, 2000).

### Plk1 and Sak Polo-boxes are Structurally Distinct—One Motif, Two Folds

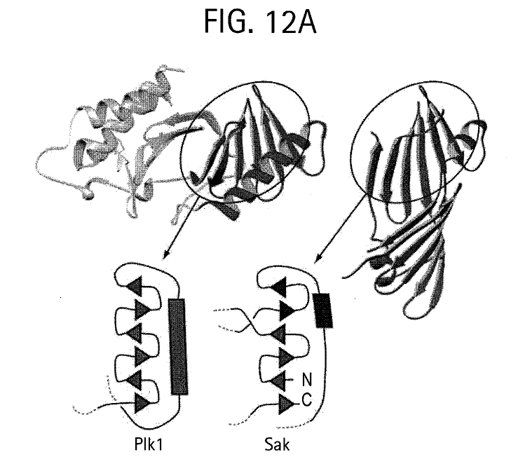

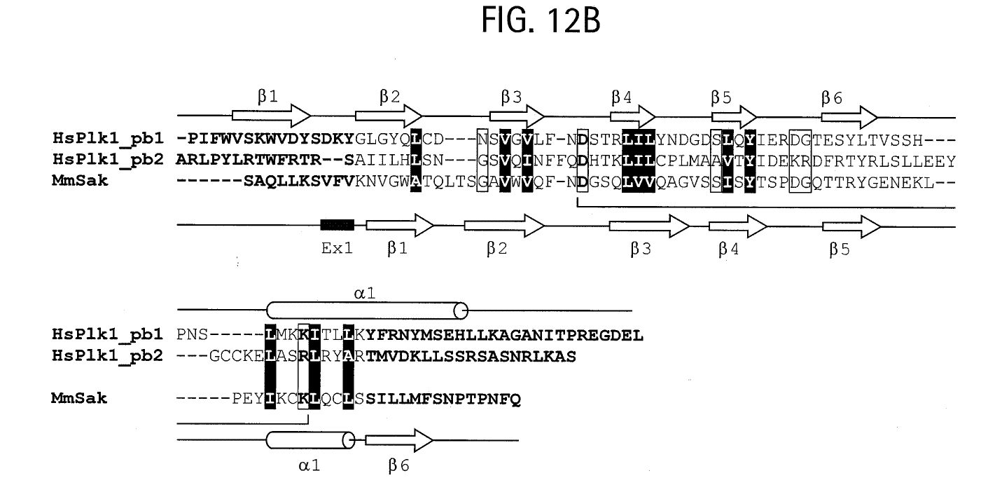

The human Plk family encompasses the canonical kinases (Plks 1-3) and Sak, which contains a highly homologous Ser/Thr kinase domain but only a single divergent Polo-box. Recent structural data has shown that the isolated Polo-box from murine Sak forms an intermolecular dimer, leading to the suggestion that tandem Polo-boxes in Plk1-related Plks may form a related, intra-molecular ‘dimeric’ architecture (Leung et al., Nat. Struct. Biol. 9:719-724, 2002). Our structure shows that this notion is broadly correct. In each case, the Polo-box repeat comprises a six-stranded β-sheet and α-helix. This structural unit associates with a second Polo-repeat via intra- or intermolecular interactions in Plk1 and Sak respectively, to form β-sandwich domain structures. However, closer examination reveals profound differences between the organizations of the two structures (FIGS. 12A and 12B). The β₆α topology of the Plk1 Polo-box is replaced by a circularly-permuted β₅αβ topology in Sak. Consequently, Plk1 β1 has no equivalent in the Sak Polo-box sequence, and instead overlaps structurally with Sak β6. In addition, the Sak β-sheet is completed by a ‘segment-swap’ of β4 & 5 between monomers. Most strikingly, the association of the two Polo-boxes differs completely such that residues forming the interface between Polo-repeats in the Sak homodimer are located largely on the exterior of the Plk1 β-sandwich, where they partially form the interface with the flanking α-helical segments.

Mutation of the His-Lys Pincer Abolishes Phosphopeptide Binding in vitro, Cdc25 Binding in vivo, and Centrosomal Localization of the Plk1 PBD

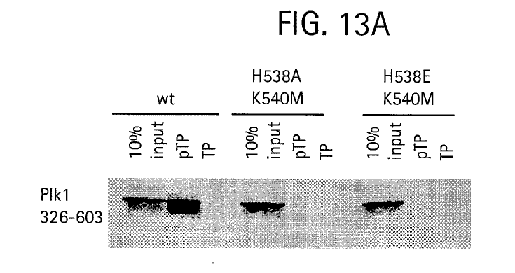

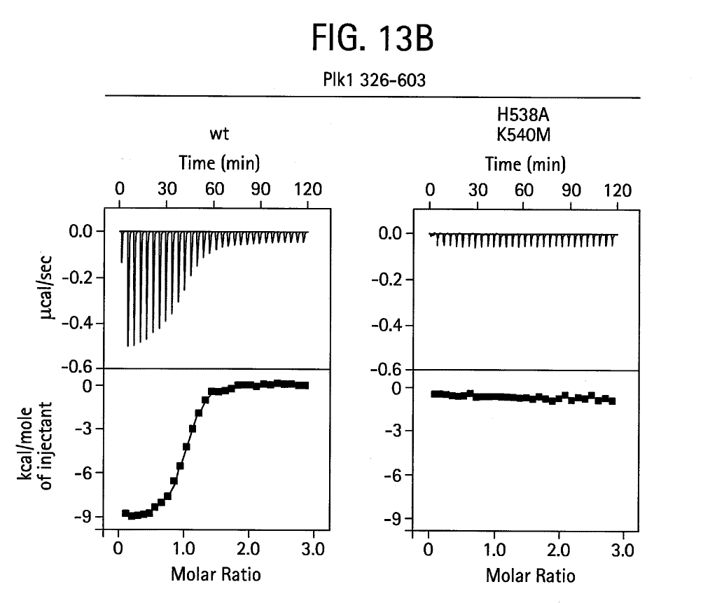

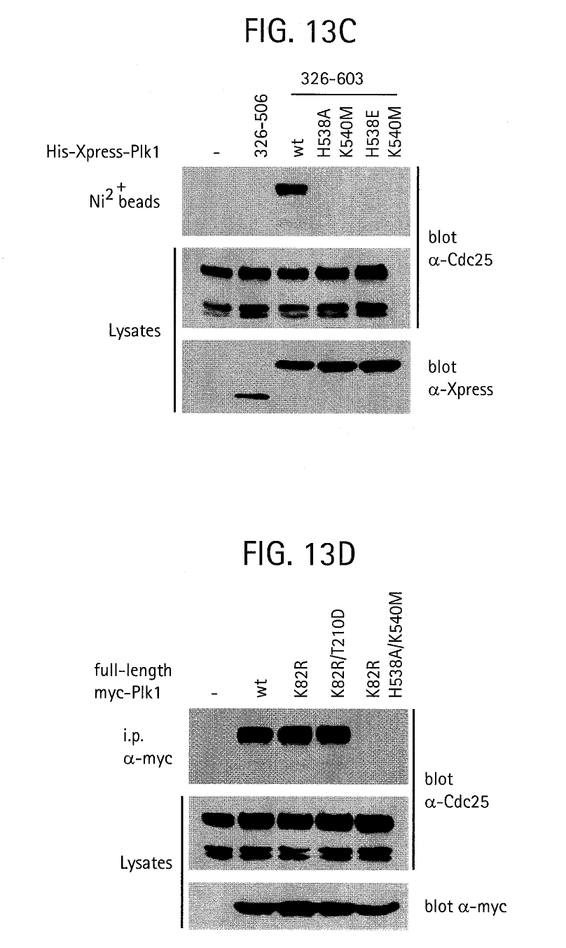

To verify that the key phosphothreonine-interacting residues identified in the X-ray crystal structure were indeed responsible for mediating phospho-dependent interactions in vitro and in vivo, we mutated His-538 and Lys-540 of the pThr pincer motif, to either Ala and Met, or Glu and Met, respectively. These mutations severely disrupt phosphopeptide binding in solution as judged by the reduced binding of in vitro translated Plk1 PBD to a bead-immobilized pThr-Pro oriented library (FIG. 13A) and by ITC (FIG. 13B).

During mitotic entry, Cdc2/Cyclin-B and Plk1 cooperate to activate the dual specificity phosphatase Cdc25 through extensive phosphorylation of its N-terminus as part of an amplification loop for Cdc2/Cyclin-B activation (Abrieu et al., J. Cell. Sci. 111:1751-1757, 1998; Hoffmann et al., EMBO J. 12:53-63, 1993; Izumi et al., Mol. Biol. Cell 4:1337-1350, 1993; Izumi et al., Mol. Biol. Cell 6:215-226, 1995; Kumagai et al., Cell 70:139-151, 1992; Kumagai et al., Science 273:1377-1380, 1996; Qian et al., Mol. Cell. Biol. 19:8625-8632, 1999; Qian et al., Mol. Biol. Cell 12:1791-1799, 2001). Mitotically phosphorylated Cdc25C exhibits a large mobility shift on SDS-PAGE (Kumagai et al., Cell 70:139-151, 1992). Cdc25C is phosphorylated on at least five Ser/Thr-Pro sites by Cdc2/Cyclin-B in vitro (Izumi et al., Mol. Biol. Cell 4:1337-1350, 1993; Strausfeld et al., J. Biol. Chem. 269:5989-6000, 1994). One of these sites, Thr-130, occurs within a near-optimal PBD binding motif, Leu-Leu-Cys-Ser-pThr-Pro-Asn (SEQ ID NO: 53). We previously observed that a GST-fusion of the isolated PBD could pull-down wild-type Cdc25C, but not a T130A or S129V Cdc25C mutant, from mitotically-arrested HeLa cell lysates. These data strongly suggested that Cdk priming of Thr-130 generates a binding site for the Plk1 PBD to facilitate full activation of Cdc25C by subsequent Plk1-mediated phosphorylation (Elia et al., Science 299:1228-1231, 2003). As shown in FIG. 13C, expression of His-Xpress-tagged wild-type Plk1 PBD in vivo results in a strong interaction with the mitotically phosphorylated form of endogenous Cdc25C in nocodazole-arrested HeLa cells. However, expression of the His-538/Lys-540 pincer mutants eliminates Cdc25C binding as also observed in cells transfected with a PBD construct lacking the second Polo-box.

To investigate whether the PBD plays a similar substrate-targeting role in the context of full-length Plk1, HeLa cells were transfected with myc-tagged wild-type or mutant constructs of full-length Plk1, and interactions between Plk1 and endogenous Cdc25C examined in nocodazole-arrested cells using immunoprecipitation and Western blotting (FIG. 13D). We observed a strong in vivo interaction between the mitotically upshifted form of endogenous Cdc25C with full-length Plk1 in arrested cells that, somewhat surprisingly, was not increased when a kinase-dead Plk1 mutant (K82R) or a double mutant incorporating a T210D mutation in the T-loop to further expose the kinase-binding cleft were employed as substrate traps. Conversely, mutation of the His-538/Lys-540 phosphate pincer mechanism in full-length Plk1 completely disrupted the in vivo interaction between Plk1 and Cdc25C demonstrating that the interaction of full-length Plk1 with full-length Cdc25 in G2/M-arrested cells is mediated primarily through the PBD, rather than its associated the kinase domain. This result is important since it directly demonstrates a requirement for PBD phosphopeptide-binding in substrate targeting in the context of the full-length Plk1 molecule.

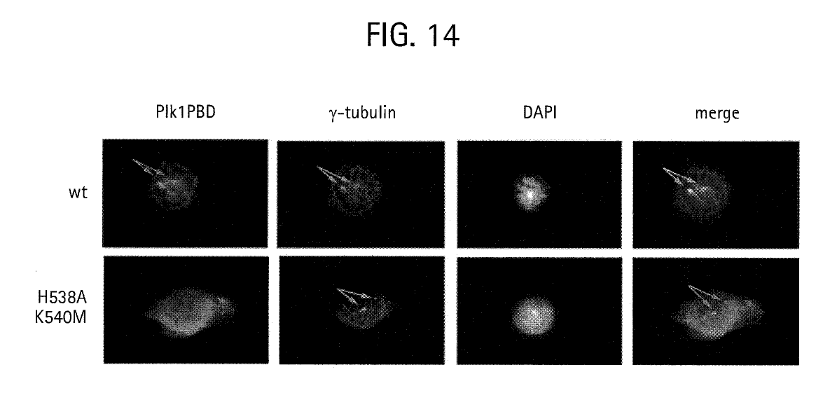

Finally, we observed that mutation of the His-538/Lys-540 pincer eliminates targeting of the Plk1 PBD to centrosomes in permeabilized prophase-arrested cells (FIG. 6). This finding suggests that the localization of Plk1 to centrosomes observed in vivo (Jang et al., Proc. Natl. Acad. Sci. USA 99:1984-1989, 2002; Lee et al., Proc. Natl. Acad. Sci. USA 95:901-9306, 1998) results from direct interactions between the PBD and phosphorylated centrosomal components. In summary, the results in FIGS. 13 and 14 show conclusively that the structurally defined His-538/Lys-540 pincer mechanism that is responsible for mediating phosphopeptide binding in vitro, plays a similar critical role in substrate targeting in vivo.

### Phosphodependent Substrate Recognition is Necessary for the Disruption of Mitotic Progression by the Isolated Plk1 PBD

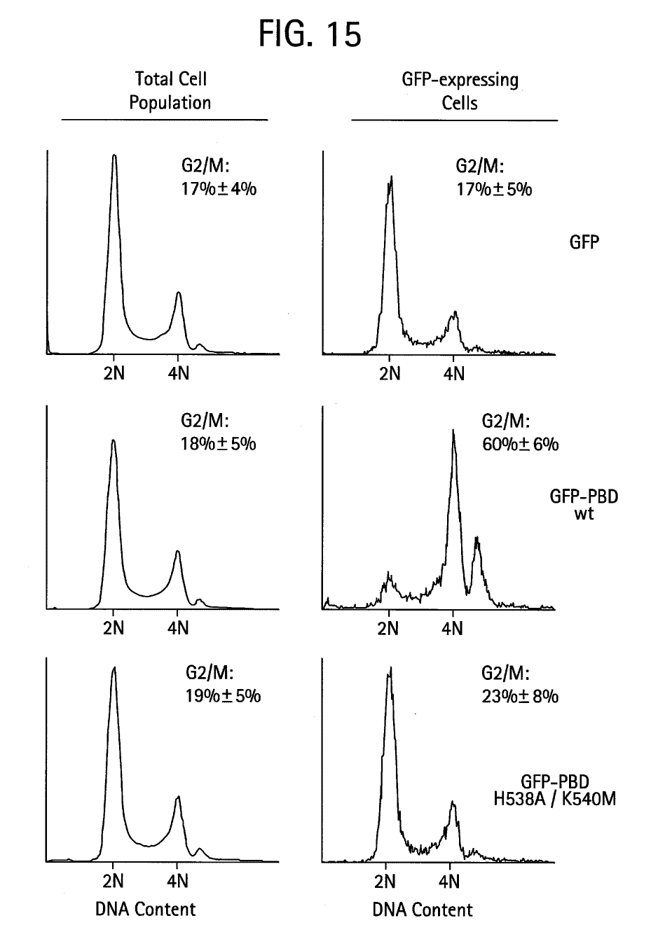

Since the PBD is necessary for targeting Plk1 to primed substrates, its overexpression might be expected to act in a dominant-negative fashion to inhibit correct localization of endogenous Plk1 and, therefore, disrupt Plk1 function in vivo. Indeed, overexpression of the C-terminus of Plk1 has been shown to cause mitotic arrest and induce formation of randomly oriented, disorganized spindles (Jang et al., Proc. Natl. Acad. Sci. USA 99:1984-1989; Seong et al., J. Biol. Chem. 277:32282-32293, 2002). The X-ray structure of the PBD-phosphopeptide complex now enables us to dissect the role of phospho-specific binding in this phenotype. In agreement with previous studies, we found that overexpression of a GFP-fusion of the Plk1 PBD in HeLa cells caused a dramatic increase in the population of cells in G2/M (60% for PBD-GFP— vs. 17% for GFP-expressing cells) (FIG. 15). Importantly, this accumulation of mitotic cells was abolished by mutation of His-538 and Lys-540 (23% in G2/M). In addition, expression of the wild-type PBD-GFP construct induced aneuploidy in HeLa cells, evident as a peak of cells with DNA content >4N, in agreement with anti-Plk1 antibody microinjection studies reported by Lane and Nigg (Lane et al., J. Cell. Biol. 135:1701-1713, 1996). However, this effect was completely lost when the His/Lys pincer mutant was employed. The dominant negative effects strongly suggest that phosphopeptide-binding by the PBD in full-length Plk1 normally plays a role in both proper mitotic progression and in the establishment of a functional bipolar spindle to ensure equal chromosome segregation.

### Phosphopeptide Binding to the PBD Stimulates Plk1 Kinase Activity

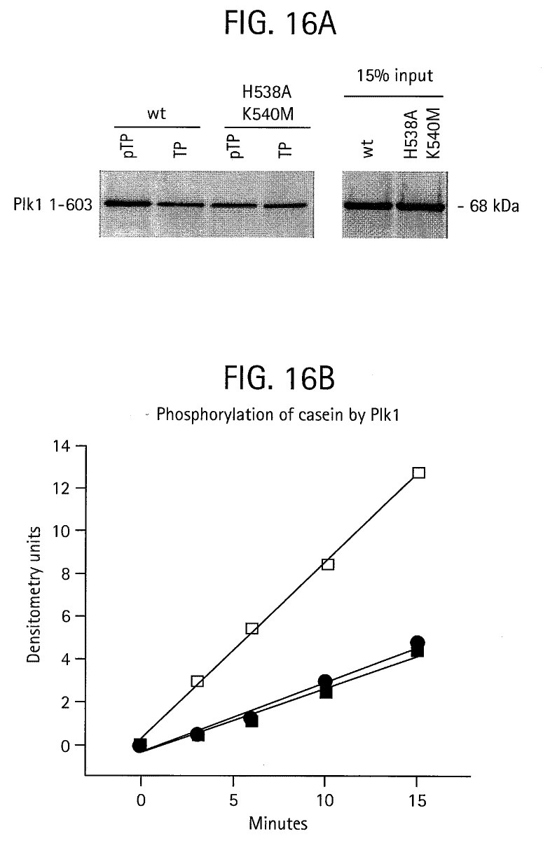

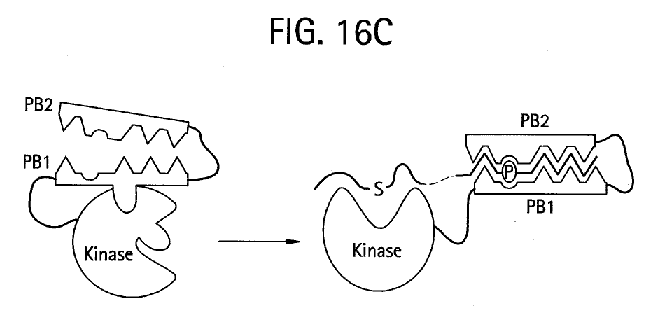

Lee and Erikson (Lee et al., Mol. Cell. Biol. 17:3408-3417, 1999) and Mundt et al. (Biochem. Biophys. Res. Commun. 239:377-385, 1997) observed that deletion of the C-terminus of Plk1 increased the kinase activity ˜3-fold while Jang et al (Jang et al., Proc. Natl. Acad. Sci. USA 99:1984-1989, 2002) found that the isolated Plk1 C-terminus interacts with and inhibits the activity of the isolated kinase domain towards the exogenous substrate casein. We observed the complementary result, namely that the kinase domain appears to inhibit phosphopeptide binding by the PBD. While the isolated Plk1 PBD binds strongly and specifically to pSer/pThr-containing peptides (FIG. 13A), phosphopeptide binding by the PBD within full-length Plk1 is reduced at least 10-fold, and is considerably less phospho-dependent (FIG. 16A, wt lanes). The phospho-specific binding component of full-length Plk1 is clearly mediated by the PBD (FIG. 16A, compare wt pTP and TP lanes with H538A/K540M pTP and TP lanes). This suggested that a mutually inhibitory interaction exists between the Plk1 PBD and the kinase domain in full-length Plk1.

We wondered whether binding of the PBD to phosphopeptides was sufficient to relieve this intramolecular interaction and stimulate the activity of the kinase domain towards exogenous substrates. Baculovirally-produced Plk1 was therefore incubated with either the optimal PBD phosphopeptide or its non-phosphorylated counterpart and kinase activity towards casein measured by SDS-PAGE/autoradiography. As shown in FIG. 16B, addition of the optimal PBD phosphopeptide increased Plk1 kinase activity by a factor of 2.6, while addition of the non-phosphorylated peptide had no effect. This result compares quite favourably with the ˜2.5-fold stimulation of Src and Hck kinase activity that is observed when these full-length Src family kinases are incubated with their optimal SH2-binding phosphotyrosine peptides to relieve SH2-mediated inhibition of the kinase domain (Liu et al., Oncogene 8:1119-1126, 1993; Moarefi et al., Nature 385:650-653, 1997). Thus, our results for Plk1 suggested that binding of the PBD to primed phosphorylation sites not only serves to target the kinase domain to substrates but also simultaneously activates the kinase domain for substrate phosphorylation by relieving an inhibitory intramolecular interaction (FIG. 16C).

In this study, we have elucidated a conserved phosphopeptide-binding motif that is recognized by the PBDs of all canonical members in the human Plk family, Xenopus Plx1 and S. cerevesiae Cdc5p. The high-resolution X-ray structure of the Polo-box domain bound to an optimal phosphothreonine peptide, provides a molecular rationale for motif selection, defines a new protein fold, and illustrates a unique mechanism for phospho-dependent ligand binding involving the participation of ordered solvent molecules, together with a conserved His/Lys pincer motif. We have identified a pSer/Thr-dependent mechanism of Plk activation in which intramolecular inhibition of the kinase by the PBD is relieved by PBD interaction with pre-phosphorylated binding targets.

### Structural Definition of the Polo-Box Domain: A General Phosphoprotein Recognition Module

Previous reports have described the presence of 1-3 Polo-boxes within the C-terminal regions of Polo-like kinases (Glover et al., Genes Dev. 12:3777-3787, 1998; Glover et al., J. Cell. Biol. 135:1681-1684, 1996; Nigg, Curr. Opin. Cell. Biol. 10:776-783, 1998; Seong et al., J. Biol. Chem. 277:32282-32293, 2002). Our structure now definitively shows that the PBD consists of two structurally homologous regions corresponding to two conserved Polo-box sequences. Phosphopeptide binding occurs at the interface of the two Polo-boxes, rationalizing both the observed 1:1 stoichiometry of PBD/ligand binding (FIG. 5B) and the requirement for both Polo-boxes for efficient subcellular localization of Plk1 in vivo (Seong et al., J. Biol. Chem. 277:32282-32293, 2002). Polo-box Domains (PBDs) now join an expanding family of phosphoserine/phosphothreonine binding domains that includes 14-3-3 proteins, WW, FHA, WD40, and Smad MH2 domains (Yaffe et al., Curr Opin Cell Biol 13:131-138, 2001; Yaffe et al., Structure 9:R33-38, 2001). In contrast to other more ubiquitous phosphodependent binding modules, PBDs occur only in Polo-like kinases where they localize Plks to specific subcellular organelles and mitotic structures (Jang et al., 2002; Lee et al., Proc. Natl. Acad. Sci. USA 95:9301-9306, 1998; (Lee et al., Mol Cell Biol 17, 3408-3417, 1999) and target the kinase to substrates that have been primed by prior phosphorylation.

### Common Phosphopeptide Motif Selection by the PBD Family

In higher eukaryotes, different Plk family members function at different points in the cell cycle (Donaldson et al., 2001; Glover et al., Genes Dev 12:3777-3787, 1998; Glover et al., J Cell Biol 135, 1681-1684, 1996; Ma et al., Mol Cancer Res 1, 376-384, 2003; Nigg, Curr Opin Cell Biol 10:776-783, 1998) or play antagonistic roles in response to DNA damage (Bahassi et al., Oncogene 21, 6633-6640, 2002; Smits et al., Nat Cell Biol 2:672-676, 2000; Xie et al., Cell Cycle 1:424-429, 2002). Given the similarity in the selected motifs with a Ser-pSer/pThr-Pro/X core for these three proteins, potential mechanisms to separate Plks within a single organism achieve substrate specificity might include different substrate selectivities by their respective kinase domains, spatially and temporally restricted activation of Plks by upstream kinases, or the well documented cell-cycle regulation of Plk1 and 2 expression (Golsteyn et al., Cell Sci 107:1509-1517, 1994; Lee et al., 1995; Ma et al., Mol Cancer Res 1:376-384, 2003). One pathway in which such specificity must be vital is the DNA damage response, since Plk1 is inhibited by DNA damage (Smits et al., Nat Cell Biol 2:672-676, 2000), while Plk3 appears to be activated (Xie et al., Cell Cycle 1:424-429, 2002).

In addition to pThr-1 selectivity for serine, all PBDs that we have examined exhibit moderate specificity for proline at the pThr+1 position, emphasizing a central role for CDKs and other proline-directed kinases in priming substrates for Plk1 targeting. Several lines of evidence support this model. For example, maximal Plk1-induced activation and nuclear translocation of Cdc25 has been shown to require cyclin B coexpression (Toyoshima-Morimoto et al., EMBO Rep. 3:341-348, 2002). Furthermore, full reconstitution of purified APC activity requires prior synergistic phosphorylation of the APC by both Cdc2 and Plk1 (Golan et al., J. Biol. Chem. 277:15552-15557, 2002). Interestingly, the backbone torsion angles of the trans-proline in the Plk1-bound phosphopeptide are very similar to those of the equivalent Pro residue in the ternary cyclinA3/CDK2/peptide complex structure (Brown et al., Nat. Cell. Biol. 1:438-443, 1999). Thus, the conformation of the peptide in the PBD complex reflects not only the structural requirements for Plk interaction but also the requirements for the initial priming phosphorylation.

Nevertheless, a clear tolerance for residues other than proline demonstrates that other mitotic kinases may also serve as priming agents. In this regard, the NIMA-related kinase Finl has been recently shown to increase Plol affinity for spindle pole bodies in S. pombe (Grallert et al., EMBO J. 21:3096-3107, 2002). Identification of substrates for Plk family members, as well as the kinases involved in substrate priming is, therefore, important.

### The Structural Basis of Phosphopeptide Binding

The PBD binds to phosphorylated epitopes in a way that is distinct from that observed previously in structures of other protein-phosphopeptide complexes (Yaffe et al., Structure 9:R33-38, 2001). These differences include the His/Lys pincer, a significant contribution from bridging water molecules and an unusual orientation of the pThr-1 residue that is directed toward the protein-binding surface. Although stereospecific, solvent-mediated binding has been described in other systems, ‘solvent-bridged’ interactions with the phosphoryl group have not been observed in any structures of protein-phosphopeptide complexes reported to date. Rather, the phospho moiety is always held by direct interactions, most often with highly conserved arginine side-chains (Eck et al., Nature 362:87-91, 1993; Waksman et al., Nature 358:646-653, 1992; Yaffe et al., Structure 9:R33-38, 2001). The importance of the His/Lys pincer in the Plk1 PBD structure is exemplified by our observations that its mutation abrogates phosphopeptide binding by the PBD in vitro, targeting of Plk1 to Cdc25C in vivo, and centrosomal localization, as well as disrupt the ability of the isolated PBD to induce G2/M arrest and aberrant spindle function.

Structure-based sequence alignments (FIG. 12B) show that the binding surface formed at the interface of the two Polo-boxes is the only totally conserved region in the PBD, further supporting our finding that the PBDs from different Plks generally select very similar optimal phosphopeptide binding motifs. Crucial hydrogen-bond interactions and van der Waals contacts with Trp-414 of Plk1 rationalize both the strong serine selection at the (pThr/pSer)-1 position and the fact that mutation of Trp-414 disrupts Plk1 function in vivo (Lee et al., Proc. Natl. Acad. Sci. USA 95:9301-9306, 1998). The absolute conservation of Trp-414 predicts that all family members should exhibit the same serine preference, and we now show that this is the case. Historically, the 10 amino acid sequence surrounding Trp-414 was considered the signature motif for the non-catalytic region of Polo-family kinases (Golsteyn et al., Cell Sci. 107:1509-1517, 1994).

### Comparison of the Plk1 PBD and Sak Polo-Box Structures

The Plk1 PBD and Sak Polo-box structures emphasize how related sequence motifs are able to form markedly different protein folds. Significant structural differences between homologous proteins have been observed only rarely and most prominently in the KH family of small RNA-binding domains (Grishin, Nucleic Acids Res. 29:638-643, 2001 and references therein). In this case, two distinct sub-families of structures are distinguishable by different topologies of α and β secondary structural elements although all share a related hydrophobic core and similar overall tertiary structure. The differences between the Plk1 PBD and Sak Polo-box are more extreme and emphasize how related sequence motifs are able to form markedly different protein folds. This, in turn, has considerable implications for both motif-based structure prediction and efforts to delineate biological function from structures of apparently homologous proteins.

How do these unexpected structural differences relate to PBD function in Plk1 and Polo-box function in Sak subfamily Plks? The grossly different architectures argue against conservation of the phosphoprotein-binding function since residues most intimately involved in phosphopeptide binding by Plk1 (e.g. His-538/Lys-540, Trp-414) are not conserved in Sak. Furthermore, examination of the electrostatic potential surface of the Sak Polo-box dimer shows no significant regions of positive charge (data not shown), a property otherwise common to phospho-dependent binding proteins.

### A Model for Phospholigand-Induced Stimulation of Plk Kinase Activity

Two alternative models for intramolecular regulation of kinase activity by a phosphopeptide binding domain are exemplified by the mechanisms of SH2 domain-mediated inhibition in Src family kinases and SHP-family tyrosine phosphatases. In the Src-type model, the phosphopeptide binding cleft of the SH2 domain engages an internal phosphotyrosine motif at the C-terminus of the molecule to hold the kinase domain in an inactive conformation (Sicheri et al., Nature 385:602-609, 1997; Xu et al., Nature 385:595-602, 1997). We believe that Plk1 does not operate through this mechanism since it does not possess an internal optimal PBD binding site, and interaction of the PBD with the Plk1 kinase domain is not dependent on phosphorylation (Jang et al., Proc. Natl. Acad. Sci. USA 99:1984-1989, 2002). In fact, mutation of Thr-210 to Asp as a mimic of kinase activation loop phosphorylation, actually abolishes PBD binding (Jang et al., Proc. Natl. Acad. Sci. USA 99:1984-1989, 2002). Furthermore, mutation of Trp-414 in Polo-box 1 has been shown to have no effect on the basal level of Plk1 kinase activity (Lee et al., Proc. Natl. Acad. Sci. USA 95:9301-9306, 1998). Since mutations at this position disrupt phosphodependent PBD interactions, it would seem that kinase regulation occurs through a phospho-independent binding function of the PBD.

In the SHP2 model, binding of the back surface of the N-terminal SH2 domain to the phosphatase domain partially occludes the catalytic cleft and simultaneously deforms the SH2 domain's binding pocket to reduce its affinity for phosphopeptide ligands (Hof et al., Cell 92:441-450, 1998). This is entirely consistent with the reduced phosphopeptide binding that we observe for the PBD in the context of full-length Plk 1 (FIG. 8A, 8C). In the case of SHP2, high local concentrations of phosphotyrosine ligands are able to bind to the N-terminal SH2 domain, inducing a concomitant conformational rearrangement of the SH2 binding cleft that is transmitted to its phosphatase-interacting surface and releases the catalytically competent phosphatase domain. We believe Plks may be regulated by a related mechanism (FIG. 8C). Some support for the SHP-like mechanism arises from our observation that the N-terminal Polo-box of one molecule in the crystallographic asymmetric unit that is not involved in extensive lattice contacts displays significantly higher temperature factors than its C-terminal counterpart (58 Å² vs 37 Å²). This implies a rather dynamic association of the two Polo-boxes that is likely to be more pronounced in the absence of the phosphopeptide ligand. In our current model, binding of the phosphopeptide between the N- and C-terminal Polo motifs acts as a structural switch, stabilizing a conformation of the PBD that is inappropriate for association with the kinase domain. Subsequent T210D phosphorylation by upstream kinases would then serve to maintain the active state by preventing re-binding of the PBD to the kinase. Definitive proof of this mechanism will require the determination of structures of full-length Plk's and their complexes. This work is in progress.

It is clear that proper mitotic progression requires the highly regulated interplay between CDK's and a variety of other proteins kinases such as Aurora, NIMA, and Polo-like kinases, yet the molecular events that underlie the activity of many of these enzymes are largely unknown. The results of our integrated biochemical, structural and cell-biological approach now provide a framework within which the cellular function of the Polo-box motif can be understood. Plk1 is overexpressed in a variety of human tumors (Strebhardt et al., JAMA 283:479-480, 2000; Takai et al., Cancer Lett. 169:41-49, 2001), and down-regulation of human Plk1 has been shown to inhibit proliferation of cultured tumor cells (Elez et al., Biochem. Biophys. Res. Commun. 269:352-356, 2000; Liu et al., Proc. Natl. Acad. Sci. USA 100:5789-5794, 2003), suggesting that Plks are potentially important targets for therapeutic intervention. Here, we have shown that the Plk1 PBD binds to phosphorylated epitopes in a way that is distinct from any observed previously in structures of other protein-phosphopeptide complexes. The unique pattern of interactions with the Ser-pThr dipeptide suggest this motif may be employed as a useful template for the design of anti-proliferative inhibitors specifically directed against Polo-box domains. The experiments described above were carried out using the following methods.

Phospho-Motif Screen for Phosphoserine/Threonine Binding Domains

A phospho-motif-biased peptide library and its unphosphorylated counterpart were constructed as follows: biotin-Z-Gly-Z-Gly-Gly-Ala-X-X-B-X-pThr-Pro-X-X-X-X-Ala-Lys-Lys-Lys SEQ ID NO: 30 and biotin-Z-Gly-Z-Gly-Gly-Ala-X-X-B-X-Thr-Pro-X-X-X-X-Ala-Lys-Lys-Lys SEQ ID NO: 31, where pThr is phosphothreonine, Z indicates aminohexanoic acid, X denotes all amino acids except Cys, and B is a biased mixture of the amino acids P, L, I, V, F, M, W. Streptavidin beads (Pierce, 75 pmol/μL gel) were incubated with a five-fold molar excess of each biotinylated library in 20 mM Tris/HCl (pH7.5), 125 mM NaCl, 0.5% NP-40, 1 mM EDTA and washed four times with the same buffer to remove unbound ligand. The bead-immobilized libraries (30 μL gel) were added to 6 μL of an in vitro translated [³⁵S]-labeled protein pool in 200 μL binding buffer (20 mM Tris/HCl (pH7.5), 125 mM NaCl, 0.5% NP-40, 1 mM EDTA, 1 mM DTT, 4 μg/mL pepstatin, 4 μg/mL aprotinin, 4 μg/mL leupeptin, 200 μM Na₃VO₄, 50 mM NaF). Each pool consisted of ˜30 radiolabeled proteins produced by coupled in vitro transcription/translation (Promega) of a plasmid pool containing ˜100 cDNA clones from a unidirectional and oligo dT-primed human HeLa cell library in pcDNA3.1 (Kanai et al., EMBO J. 19:6778-6791, 2000). After incubation at 4° C. for 2-3 hours, the beads were rapidly washed four times with binding buffer prior to separation on SDS-PAGE (11.4%) and autoradiography. Positively scoring hits within pools were recognized as protein bands that interacted more strongly with the phosphorylated immobilized library than its unphosphorylated counterpart. Pools containing positively scoring clones were progressively subdivided using a 96-well format and re-screened for phospho-binding until single clones were isolated and identified by DNA sequencing.

Cloning, Expression, and Purification of Plk-1 PBD Proteins

For deletion mapping of the PBD, C-terminal fragments of Plk-1 were generated by PCR and cloned into the EcoRI and XhoI sites of pcDNA3.1 (Invitrogen). For production of recombinant PBD as a GST fusion in bacteria, the 326-603 fragment of Plk-1 was ligated into the EcoRI and XhoI sites of pGEX-4T (Pharmacia), transformed into BL21, and induced in late log-phase cells at 37° C. for 3.5 hours in the presence of 0.4 mM IPTG. For measurements of peptide binding affinity by ITC, GST-Plk-1 (326-603) was isolated from bacterial lysates using glutathione agarose, cleaved from GST using thrombin (10 U/mL), and purified by anion exchange chromatography (Q Sepharose HP, Pharmacia).

Peptide Library Screening

Phosphothreonine- and phosphoserine-oriented degenerate peptide libraries containing the sequences Met-Ala-X-X-X-X-pThr-Pro-X-X-X-X-Ala-Lys-Lys-Lys SEQ ID NO: 46(theoretical degeneracy (td)=1.7×10¹⁰), Met-Ala-X-X-X-X-pThr-X-X-X-X-Ala-Lys-Lys-Lys SEQ ID NO: 47(td=1.7×10¹⁰), Met-Ala-X-X-X-X-Ser-pThr-X-X-X-X-Ala-Lys-Lys-Lys SEQ ID NO: 48(td=1.7×10¹⁰), Met-Ala-X-X-X-pSer-Pro-X-X-X-Ala-Lys-Lys-Lys SEQ ID NO: 49(td=4.7×10⁷), Met-Ala-X-X-X-X-pSer-X-X-X-X-Ala-Lys-Lys-Lys SEQ ID NO: 50(td=1.7×10¹⁰), and Met-Ala-X-X-X-X-Ser-pSer-X-X-X-X-Ala-Lys-Lys-Lys SEQ ID NO: 54(td=1.7×10¹⁰) were synthesized using N-α-FMOC-protected amino acids and standard BOP/HOBt coupling chemistry. Peptide library screening was performed using 100 μl of glutathione beads containing saturating amounts of GST-Plk-1 (residues 326-603) fusion protein (−1-1.5 mg) as described in Yaffe & Cantley (Methods Enzymol., 328:157-170, 2000). Beads were packed in a 1 mL column and incubated with 0.5 mg of the peptide library mixture for 10 minutes at room temperature in PBS (150 mM NaCl, 3 mM KCl, 10 mM Na₂HPO₄, 2 mM KH₂PO₄, pH 7.2). Unbound peptides were removed from the column by two rapid washes with PBS containing 0.5% NP-40 and two subsequent washes with PBS. Bound peptides were eluted with 30% acetic acid for 10 minutes at room temperature, lyophilized, resuspended in H₂O, and sequenced by automated Edman degradation on a Procise protein microsequencer. Selectivity values for each amino acid were determined by comparing the relative abundance (Mole percentage) of each amino acid at a particular sequencing cycle in the recovered peptides to that of each amino acid in the original peptide library mixture at the same position.

Isothermal Titration Calorimetry

Peptides were synthesized by solid phase technique with two C-terminal lysines to enhance solubility, purified by reverse phase HPLC following deprotection, and confirmed by MALDI-TOF 9 Matrix-assisted laser desorption/ionisation-time of flight mass spectrometry. Some peptides contained an additional tyrosine residue to facilitate concentration determination by optical absorbance. calorimetry measurements were performed using a VP-ITC microcalorimeter (MicroCal Inc., Studio City, Calif.). Experiments involved 10 μL injections of peptide solutions (150 μM-180 μM) into a sample cell containing 15 μM Plk-1 PBD (residues 326-603) in 50 mM Tris/HCl (pH 8.1), 200 mM NaCl, 2 mM TCEP. Thirty injections were performed with a spacing of 240 s and a reference power of 25 μCal/s. Binding isotherms were plotted and analyzed using Origin Software (MicroCal Inc. Studio City, Calif.).

Plk-1 PBD Binding to Cellular Substrates

HeLa cells were arrested in interphase or G2/M by treatment with aphidicolin (5 μg/mL) or nocodazole (50 ng/mL), respectively, for 16 hours. Cells were lysed in 25 mM Tris/HCl (pH 7.5) containing 125 mM NaCl, 0.5% NP-40, 5 mM EDTA, 2 mM DTT, 4 μg/mL pepstatin, 4 μg/mL aprotinin, 4 μg/mL leupeptin, 1 mM Na₃VO₄, 50 mM NaF, and 1 viM microcystin, and 150 μgs of lysate incubated with 10 μL of glutathione agarose beads containing 2-5 μg of GST-Plk-1 (residues 326-603), GST-Pin1, or GST for 30 minutes at 4° C. Beads were washed four times with lysis buffer. Precipitated proteins were eluted in sample buffer and detected by blotting with monoclonal MPM-2 (Upstate Biotechnology, Inc.) or polyclonal anti-Cdc25C (Santa Cruz Biotechnology, Santa Cruz, Calif.). For peptide competition experiments, GST-Plk-1 (residues 326-603) was immobilized on glutathionine beads and preincubated with 320 μM of PoloBoxtide-optimal, −8T, or −7V for 45 minutes at 4° C. For binding experiments involving mutant cdc25C, HeLa cells were transfected with wild-type and mutated versions of HA-tagged Cdc25C in pECE using Superfect (Qiagen, Valencia, Calif.). Nocodazole (50 ng/mL) was added seventeen hours after transfection and cells incubated for an additional 14 hours to arrest them in G2/M. Point mutations of Cdc25C were constructed using the QuickChange site-directed mutagenesis system (Stratagene) and verified by DNA sequencing.

Centrosomal Localization of the Plk-1 PBD

U2OS cells were cultured in 8-well chamber slides and arrested at G2/M by treatment with nocodazole (50 ng/mL) for 14 hours. After rinsing with PBS, cells were incubated with 4 μM GST-Plk-1 PBD (residues 326-603) and Streptolysin-O (1 U/ml) in permeabilization buffer (25 mM HEPES (pH 7.9), 100 mM KCl, 3 mM NaCl, 200 mM sucrose, 20 mM NaF, 1 mM NaOVO₄) for 20 minutes at 37° C. Cells were fixed in 3% paraformaldehyde/2% sucrose for 10 minutes at room temperature and extracted with a 0.5% Triton X-100 solution containing 20 mM Tris-HCl (pH 7.4), 50 mM NaCl, 300 mM sucrose, and 3 mM MgCl₂ for 10 minutes at RT. Slides were stained with Alexa Fluor 488-conjugated anti-GST (Molecular Probes, Eugene, Oreg.) and monoclonal anti-γ-tubulin (Sigma, St. Louis, Mo.) antibodies at 4° C. overnight, then stained with a Texas Red conjugated anti-mouse secondary antibody for 60 minutes at room temperature and counterstained with 4 μg/ml DAPI. Cells were examined using a Nikon Eclipse E600 fluorescence microscope equipped with a SPOT RTcamera and software (Diagnostic Instruments, Livingston, Scotland). Images were analyzed using NIH Image. For peptide competition experiments, the GST-Plk-1 PBD solution was preincubated with 250 μM of its optimal phosphopeptide ligand (PoloBoxtide-optimal) or its unphosphorylated counterpart (PoloBoxtide-8T) for 15 minutes at room temperature prior to use.

To quantitate centrosomal localization of the GST-Plk-1 PBD relative to γ-tubulin, black and white images of single cells showing comparable overall intensity for Alexa Fluor and Texas Red were selected and scaled to an average grayscale value of 200 (1=white, 255=black). The normalized intensity of centrosome-specific Alexa Fluor 488 staining (N.I._{AF488}) or Texas Red staining (N.I._{TR}) above background was defined as ([I_{centrosome}−1_{cell}]/I_{cell}) where I_{centrosome} indicates the fluorescence intensity of either Alexa-Fluor 488 or Texas Red averaged over the centrosome and I_{cell} indicates the overall fluorescence intensity averaged over the entire cell. The relative GST-PBD/γ-tubulin specific staining was then calculated as N.I._{AF488}/NI_{TR}.

### Screens to Identify Novel Binding Pairs

Novel binding pairs can be identified by the methods of the invention. For example, phosphopeptides are generated that are biased to include MAP kinase and Cell-cycle dependent kinase (Cdks) consensus phosphorylation sites (i.e., pSer-Pro), for use in screening for novel pSer-Pro binding polypeptides. Such a screen can be easily adapted to identify additional binding pairs. By taking advantage of the observation that protein kinases and phosphopeptide binding domains appear to co-evolve to recognize overlapping sequence motifs, phosphopeptides can be generated to follow specific protein kinase substrates. Thus, basophilic phosphopeptides having a core sequence including RXRSX[pS/pT] (SEQ ID NO: 55) (where R is arginine, pS is phosphoserine, pT is phosphothreonine, and X is any amino acid) can be used to identify novel binding partners dependent on the kinase, Akt. Other potential basophilic kinase substrates based on consensus phosphorylation sequences of protein kinase C (PKC), cAMP-dependent protein kinase (PKA), G-protein coupled receptor kinases such as β-ARK may also be used.

Several methods are known in the art to identify consensus kinase substrates, for example, in U.S. Pat. No. 5,532,167, U.S. Pat. No. 6,004,757, and WO 98/54577. Thus, degenerate phosphopeptides can be generated based on consensus kinase substrate peptide motifs. Exemplary kinase substrate peptide motifs that can be used include, without limitation, phosphopeptides derived from the consensus sequences of the serine/threonine kinases, Ca²⁺/calmodulin dependent kinases (CaMKs), check point kinases (e.g. CHK, Rad53), myosin light chain kinases, DRAK, Trio, casein kinase 1, cell cycle dependent kinases (CDKs, e.g., Cdc2, Cdk4, Cdk6), glycogen synthase kinases (GSK), MAP kinases (e.g., Jnk, Erk, p38), STE family kinases (e.g., PAK, GCK/MAP4K), MAP kinase activated kinases (e.g., Mnk), eIF2a kinases (e.g., PERK, PKR, HRI, GCN2), Raf kinases (e.g., A-Raf, B-Raf), casein kinase II, aurora/Polo kinases, mixed lineage kinases (e.g., MLK1, -2, -3), AKAP, Activin-receptor like kinase (Kir4), CAK, Mos, Pim, and Ksr. Other kinase substrate-derived phosphopeptide sequences that can be used in the invention include those derived from the dual specificity kinases, WEE-1, MEKs, DYRKs, Tesk, Clk, HIPK, Mps-1, TSK, and C-TAK. Dual specificity kinases also include polypeptides related to the lipid kinases FRAP, p110 PI3 Kinase, ATM, ATR, and DNA-PK.

Protein tyrosine kinase substrate peptide motifs can also be used in the invention and include phosphopeptides derived from the consensus substrate sequences of the receptor tyrosine kinases, which include the EGF-R family (e.g., EGF-R, Her2/Neu), PDGF-R, CSF—R, IGF-R, VEGF-R (e.g., Flk/Kdr, Flt), HGF-R (Met), NGF-R (e.g., TrkA, -B, -C), FGF-R, ROR, Tie-1, Tie-2/Tek, Eph (e.g., EphA₁₋₈, EphB₁₋₆), Rik, Ron, Ros, Ret, and from the cytoplasmic tyrosine kinases, which include, the Src family (e.g., Src, Lck, Lyn, Fyn, Hck, Yes), Abl, Csk, CTK, JAKs, FAK, ITK, BTK, Ack/Pyk, Tec, Tyk, Syk, Zap70, Fer, and Fes/Fps.

Binding pairs identified are not limited to those that include phosphopeptide binding domains. The methods of the invention may be used to identify virtually any peptide-binding domain in which the domain is identified by simultaneous screening of a protein/polypeptide expression library with a biased peptide library. For example, a screen for binding pairs is carried out to identify a peptide-binding domain, for example, a PDZ, SH3, or WW peptide binding domain. The “bait” peptide library contains a degenerate collection of peptides oriented around at least two or more fixed residues. A working example of such a screen is provided in the upper left panel of FIG. 9B, where there is a band at ˜24 kDa that binds the non-phosphopeptide library but not the phosphopeptide library., suggesting that it is specific for binding to BxTP motifs.

### Cloning and Expression of PBD Proteins

C-terminal fragments of human Plk1 (residues 326-603), human Plk2 (residues 355-685), human Plk3 (residues 335-646), Xenopus Plx1 (residues 317-598), and Saccharomyces cerevesiae Cdc5p (residues 357-705) were amplified from IMAGE cDNA clones or directly from S. cerevisiae chromosomal DNA by PCR and ligated into suitably digested pGEX4T-3 or pGEX-6P1 (Pharmacia). Proteins were expressed in E. coli BL21(DE3) cells and purified by glutathione-affinity chromatography. For measurements of peptide binding affinity and domain mapping experiments, proteins were cleaved from GST with either thrombin or viral protease 3C (Pharmacia-LKB, Peapack, N.J.) and further purified by anion exchange chromatography (Q Sepharose HP, Pharmacia) or gel filtration (Superdex S-75, Pharmacia, Peapack, N.J.).

### Oriented Peptide Library Screening

Phosphothreonine-oriented degenerate peptide libraries containing the sequences Met-Ala-X-X-X-X-pThr-Pro-X-X-X-X-Ala-Lys-Lys-Lys SEQ ID NO: 46(theoretical degeneracy (td)=1.7×10¹⁰) and Met-Ala-X-X-X-X-Ser-pThr-X-X-X-X-Ala-Lys-Lys-Lys SEQ ID NO: 48(td=1.7×10¹⁰) were synthesized using N-α-FMOC-protected amino acids and standard BOP/HOBt coupling chemistry. Peptide library screening was performed using 100 μl of glutathione beads containing saturating amounts (−1-1.5 mg) of GST-hPlk1, GST-hPlk2, GST-hPlk3, GST-Plx1, or GST-Cdc5p as described previously (Yaffe et al., Methods Enzymol 328:157-170, 2000).

### Peptide Binding Measurements

Peptides were synthesized by solid phase technique with two C-terminal lysines to enhance solubility. Some peptides contained an additional tyrosine residue to facilitate concentration determination by optical absorbance. Isothermal titration calorimetry was performed using a VP-ITC microcalorimeter (MicroCal Inc. Studio City, Calif.) by titration of 15-40 μM solutions of PBD proteins with 30×10 μl injections of 150-400W peptide in a starting volume of 1.4-2.0 ml. Binding isotherms were plotted and analyzed using Origin Software (MicroCal Inc. Studio City, Calif.). Binding of in vitro translated Plk1 PBD (wild type and mutants) to bead-immobilized pTP and TP peptide libraries was performed as described previously (Elia et al., Science 299:1228-1231, 2003). pTP and TP indicate the peptide libraries biotin-Z-Gly-Z-Gly-Gly-Ala-X-X-B-X-pThr-Pro-X-X-X-X-Ala-Lys-Lys-Lys SEQ ID NO: 30 biotin-Z-Gly-Z-Gly-Gly-Ala-X-X-B-X-Thr-Pro-X-X-X-X-Ala-Lys-Lys-Lys SEQ ID NO: 31, respectively, where pThr is phosphothreonine, Z is aminohexanoic acid, X denotes all amino acids except Cys, and B is a biased mixture of the amino acids P, L, I, V, F, M, W.

### Peptide Spot Array

An ABIMED peptide arrayer with a computer controlled Gilson diluter and liquid handling robot was used to synthesize peptides onto an amino-PEG cellulose membrane using N-α-FMOC-protected amino acids and DIC/HOBT coupling chemistry. The membrane was blocked in 5% milk/TBS-T (0.1%) for 2 hours at room temperature, incubated with 0.1 μM GST-Plk1 PBD (residues 326-603) in 5% milk, 50 mM Tris/HCl (pH 7.5), 150 mM NaCl, 2 mM EDTA, 2 mM DTT for 1 hour at room temperature and washed with TBS-T (0.1%). It was then incubated with anti-GST conjugated HRP in 5% milk/TBS-T (0.1%) for 1 hour at room temperature, washed with TBS-T (0.1%), and subjected to chemiluminescence.

### Domain Mapping and Protein Purification

Limited proteolysis of Plk1 (residues 326-603) and Cdc5p were performed using trypsin or endoproteinase Glu-C (Promega). N- and C-terminal limits were determined by Edman sequencing and electrospray mass spectrometry. DNA sequences encoding the proteolytically-defined domains were amplified by PCR and cloned into pGEX-6P1 (Cdc5p) or a version modified to allow ligation-independent cloning that also permits fusion-protein cleavage with TEV protease (Stols et al., Pro. Expr. Purif. 25:8-15 2002) (SJS—unpublished data). Recombinant PBDs were then expressed and purified as above.

### Crystallization and Structure Determination

For crystallization, the phosphopeptide MAGPMQSpTPLNGAYKK (SEQ ID NO: 56) was mixed with the Plk1 PBD fragment in a 1.5:1 stoichiometric excess and concentrated to ˜0.2 mM in a buffer containing 20 mM Tris.HCT pH 8.0/500 mM NaCl, 1 mM EDTA, 3 mM DTT. Crystals were grown by microbatch methods at 18° C. using a Douglas Instruments IMPAX 1-5 crystallization robot and belong to monoclinic space-group P2₁ (a=62.4 Å, b=79.5 Å, c=62.0 Å, β=93.26° with two complexes per asymmetric unit. Native data were collected on Station 14.1 at the SRS Daresbury using cryopreserved crystals at a temperature of 100° K. All data were reduced using the HKL suite of processing software (Otwinowski et al., Meth. Enzymol. 276:307-326, 1997). Phase information was derived from a three wavelength MAD experiment, using a single crystal of Se-methionine substituted PBD in complex with the phosphopeptide. Data for each wavelength were collected to a nominal 3.0 Å spacing on Station 14.2 at the SRS, Daresbury, UK. Ten Se sites corresponding to five sites per monomer in the asymmetric unit were located, and the phases refined using SOLVE (Terwilliger et al., Acta Crystallogr. D. Biol. Crystallogr 55:849-861, 1999). Phases were extended to ˜2.5 Å against the native data using real-space non-crystallographic symmetry averaging with solvent flattening in RESOLVE (Terwilliger et al., Acta Crystallogr. D. Biol. Crystallogr 55:849-861, 1999). These maps were readily interpretable allowing a partial model of the PBD, together with seven residues of the phosphopeptide to be built using ‘O’ (Jones et al., Acta Crystallogr. A 47:110-119, 1991). Subsequent refinement using native data to 1.9 Å was carried out using CNS (Brunger et al., Acta Crystallogr. D Biol. Crystallogr. 54:905-921, 1998) and REFMAC 5.0-ARP/wARP from the CCP4 suite. A summary of statistics for the structure solution and refinement are shown in Table 5 (amino acids 20-241 of Plk1, SEQ ID NO: 109). Residues in bold: His 538, Lys540, Trp414, and Leu491.

TABLE 5
Plk1-PBD.pdb

| | | ????---- | | |
|---|---|---|---|---|
| ┌ | ┘ o ~ | î [symbol] | CRYST1 | 62.352 |

| | |
|---|---|
| 79.518 | 61.993 90.00 93.26 90.00 P 1 21 1 |

| | | | | |
|---|---|---|---|---|
| SCALE1 | 0.016038 | 0.000000 | 0.000914 | 0.00000 |
| SCALE2 | 0.000000 | 0.012576 | 0.000000 | 0.00000 |
| SCALE3 | 0.000000 | 0.000000 | 0.016157 | 0.00000 |

| | | | | | | | | | | | | |
|---|---|---|---|---|---|---|---|---|---|---|---|---|
| ATOM | 1 | N | ALA | A | 209 | 36.401 | 10.634 | 1.405 | 1.00 | 33.71 | 7 | N |
| ATOM | 2 | CA | ALA | A | 20 | 37.156 | 9.417 | 1.828 | 1.00 | 32.78 | 6 | C |
| ATOM | 3 | CB | ALA | A | 20 | 38.634 | 9.615 | 1.623 | 1.00 | 32.91 | 6 | C |
| ATOM | 4 | C | ALA | A | 20 | 36.862 | 9.066 | 3.284 | 1.00 | 31.86 | 6 | C |
| ATOM | 5 | O | ALA | A | 20 | 36.468 | 9.924 | 4.069 | 1.00 | 32.15 | 8 | O |
| ATOM | 6 | N | LEU | A | 21 | 37.062 | 7.804 | 3.631 | 1.00 | 31.41 | 7 | N |
| ATOM | 7 | CA | LEU | A | 21 | 36.766 | 7.324 | 4.979 | 1.00 | 31.14 | 6 | C |
| ATOM | 8 | CB | LEU | A | 21 | 36.948 | 5.812 | 5.061 | 1.00 | 31.43 | 6 | C |
| ATOM | 9 | CG | LEU | A | 21 | 35.921 | 4.969 | 4.306 | 1.00 | 32.63 | 6 | C |
| ATOM | 10 | CD1 | LEU | A | 21 | 36.274 | 3.499 | 4.379 | 1.00 | 32.84 | 6 | C |
| ATOM | 11 | CD2 | LEU | A | 21 | 34.520 | 5.215 | 4.881 | 1.00 | 32.61 | 6 | C |
| ATOM | 12 | C | LEU | A | 21 | 37.637 | 8.010 | 6.018 | 1.00 | 31.08 | 6 | C |
| ATOM | 13 | O | LEU | A | 21 | 37.163 | 8.368 | 7.096 | 1.00 | 30.32 | 8 | O |
| ATOM | 14 | N | SER | A | 22 | 38.912 | 8.200 | 5.687 | 1.00 | 30.80 | 7 | N |
| ATOM | 15 | CA | SER | A | 22 | 39.852 | 8.854 | 6.589 | 1.00 | 31.39 | 6 | C |
| ATOM | 16 | CB | SER | A | 22 | 41.244 | 8.902 | 5.948 | 1.00 | 31.77 | 6 | C |
| ATOM | 17 | OG | SER | A | 22 | 42.200 | 8.363 | 6.833 | 1.00 | 35.33 | 8 | O |
| ATOM | 18 | C | SER | A | 22 | 39.378 | 10.264 | 6.935 | 1.00 | 31.00 | 6 | C |
| ATOM | 19 | O | SER | A | 22 | 39.403 | 10.669 | 8.094 | 1.00 | 30.62 | 8 | O |
| ATOM | 20 | N | ASP | A | 23 | 38.959 | 11.012 | 5.919 | 1.00 | 30.36 | 7 | N |
| ATOM | 21 | CA | ASP | A | 23 | 38.404 | 12.341 | 6.135 | 1.00 | 30.45 | 6 | C |
| ATOM | 22 | CB | ASP | A | 23 | 38.129 | 13.027 | 4.805 | 1.00 | 30.88 | 6 | C |
| ATOM | 23 | CG | ASP | A | 23 | 39.394 | 13.545 | 4.149 | 1.00 | 33.47 | 6 | C |
| ATOM | 24 | OD1 | ASP | A | 23 | 40.452 | 13.591 | 4.819 | 1.00 | 34.01 | 8 | O |
| ATOM | 25 | OD2 | ASP | A | 23 | 39.418 | 13.915 | 2.961 | 1.00 | 36.44 | 8 | O |
| ATOM | 26 | C | ASP | A | 23 | 37.126 | 12.293 | 6.974 | 1.00 | 29.75 | 6 | C |
| ATOM | 27 | O | ASP | A | 23 | 36.922 | 13.105 | 7.875 | 1.00 | 29.61 | 8 | O |
| ATOM | 28 | N | MET | A | 24 | 36.249 | 11.355 | 6.662 | 1.00 | 29.28 | 7 | N |
| ATOM | 29 | CA | MET | A | 24 | 35.024 | 11.224 | 7.432 | 1.00 | 28.49 | 6 | C |
| ATOM | 30 | CB | MET | A | 24 | 34.134 | 10.133 | 6.852 | 1.00 | 28.64 | 6 | C |
| ATOM | 31 | CG | MET | A | 24 | 32.785 | 10.050 | 7.547 | 1.00 | 29.20 | 6 | C |
| ATOM | 32 | SD | MET | A | 24 | 31.750 | 8.750 | 6.855 | 1.00 | 32.07 | 16 | S |
| ATOM | 33 | CE | MET | A | 24 | 31.461 | 9.420 | 5.196 | 1.00 | 29.51 | 6 | C |
| ATOM | 34 | C | MET | A | 24 | 35.335 | 10.920 | 8.897 | 1.00 | 28.13 | 6 | C |
| ATOM | 35 | O | MET | A | 24 | 34.693 | 11.451 | 9.793 | 1.00 | 27.58 | 8 | O |
| ATOM | 36 | N | LEU | A | 25 | 36.313 | 10.059 | 9.139 | 1.00 | 28.15 | 7 | N |
| ATOM | 37 | CA | LEU | A | 25 | 36.694 | 9.740 | 10.516 | 1.00 | 28.97 | 6 | C |
| ATOM | 38 | CB | LEU | A | 25 | 37.779 | 8.665 | 10.539 | 1.00 | 28.90 | 6 | C |
| ATOM | 39 | CG | LEU | A | 25 | 38.345 | 8.310 | 11.915 | 1.00 | 29.83 | 6 | C |
| ATOM | 40 | CD1 | LEU | A | 25 | 37.224 | 7.836 | 12.841 | 1.00 | 29.82 | 6 | C |
| ATOM | 41 | CD2 | LEU | A | 25 | 39.421 | 7.240 | 11.787 | 1.00 | 30.16 | 6 | C |
| ATOM | 42 | C | LEU | A | 25 | 37.158 | 10.988 | 11.261 | 1.00 | 28.74 | 6 | C |
| ATOM | 43 | O | LEU | A | 25 | 36.769 | 11.219 | 12.406 | 1.00 | 28.86 | 8 | O |
| ATOM | 44 | N | GLN | A | 26 | 37.971 | 11.812 | 10.602 | 1.00 | 28.76 | 7 | N |
| ATOM | 45 | CA | GLN | A | 26 | 38.480 | 13.026 | 11.236 | 1.00 | 28.88 | 6 | C |
| ATOM | 46 | CB | GLN | A | 26 | 39.463 | 13.764 | 10.309 | 1.00 | 29.72 | 6 | C |
| ATOM | 47 | CG | GLN | A | 26 | 40.667 | 12.948 | 9.920 | 1.00 | 33.49 | 6 | C |
| ATOM | 48 | CD | GLN | A | 26 | 41.649 | 13.722 | 9.050 | 1.00 | 38.46 | 6 | C |
| ATOM | 49 | OE1 | GLN | A | 26 | 41.310 | 14.150 | 7.939 | 1.00 | 41.83 | 8 | O |
| ATOM | 50 | NE2 | GLN | A | 26 | 42.864 | 13.898 | 9.546 | 1.00 | 39.82 | 7 | N |
| ATOM | 51 | C | GLN | A | 26 | 37.336 | 13.953 | 11.589 | 1.00 | 27.91 | 6 | C |
| ATOM | 52 | O | GLN | A | 26 | 37.307 | 14.528 | 12.675 | 1.00 | 27.39 | 8 | O |
| ATOM | 53 | N | GLN | A | 27 | 36.395 | 14.098 | 10.660 | 1.00 | 26.30 | 7 | N |
| ATOM | 54 | CA | GLN | A | 27 | 35.246 | 14.968 | 10.848 | 1.00 | 25.97 | 6 | C |
| ATOM | 55 | CB | GLN | A | 27 | 34.419 | 15.035 | 9.553 | 1.00 | 26.10 | 6 | C |
| ATOM | 56 | CG | GLN | A | 27 | 35.155 | 15.752 | 8.396 | 1.00 | 25.54 | 6 | C |
| ATOM | 57 | CD | GLN | A | 27 | 34.598 | 15.402 | 7.022 | 1.00 | 25.17 | 6 | C |
| ATOM | 58 | OE1 | GLN | A | 27 | 33.521 | 14.808 | 6.903 | 1.00 | 25.56 | 8 | O |
| ATOM | 59 | NE2 | GLN | A | 27 | 35.337 | 15.760 | 5.979 | 1.00 | 25.43 | 7 | N |
| ATOM | 60 | C | GLN | A | 27 | 34.366 | 14.489 | 12.005 | 1.00 | 25.75 | 6 | C |
| ATOM | 61 | O | GLN | A | 27 | 33.896 | 15.292 | 12.819 | 1.00 | 25.65 | 8 | O |
| ATOM | 62 | N | LEU | A | 28 | 34.135 | 13.184 | 12.055 | 1.00 | 25.72 | 7 | N |
| ATOM | 63 | CA | LEU | A | 28 | 33.317 | 12.590 | 13.121 | 1.00 | 26.50 | 6 | C |
| ATOM | 64 | CB | LEU | A | 28 | 32.975 | 11.134 | 12.778 | 1.00 | 26.13 | 6 | C |
| ATOM | 65 | CG | LEU | A | 28 | 31.914 | 10.996 | 11.687 | 1.00 | 26.32 | 6 | C |
| ATOM | 66 | CD1 | LEU | A | 28 | 31.749 | 9.549 | 11.289 | 1.00 | 25.22 | 6 | C |
| ATOM | 67 | CD2 | LEU | A | 28 | 30.580 | 11.563 | 12.173 | 1.00 | 26.83 | 6 | C |
| ATOM | 68 | C | LEU | A | 28 | 34.027 | 12.674 | 14.472 | 1.00 | 26.87 | 6 | C |
| ATOM | 69 | O | LEU | A | 28 | 33.417 | 13.019 | 15.488 | 1.00 | 27.36 | 8 | O |
| ATOM | 70 | N | HIS | A | 29 | 35.318 | 12.373 | 14.488 | 1.00 | 27.61 | 7 | N |
| ATOM | 71 | CA | HIS | A | 29 | 36.063 | 12.458 | 15.740 | 1.00 | 28.39 | 6 | C |
| ATOM | 72 | CB | HIS | A | 29 | 37.530 | 12.070 | 15.579 | 1.00 | 28.75 | 6 | C |
| ATOM | 73 | CG | HIS | A | 29 | 38.329 | 12.314 | 16.819 | 1.00 | 31.08 | 6 | C |
| ATOM | 74 | ND1 | HIS | A | 29 | 38.125 | 11.598 | 17.978 | 1.00 | 31.37 | 7 | N |
| ATOM | 75 | CE1 | HIS | A | 29 | 38.939 | 12.045 | 18.917 | 1.00 | 32.66 | 6 | C |
| ATOM | 76 | NE2 | HIS | A | 29 | 39.647 | 13.041 | 18.417 | 1.00 | 31.82 | 7 | N |
| ATOM | 77 | CD2 | HIS | A | 29 | 39.279 | 13.236 | 17.107 | 1.00 | 32.90 | 6 | C |
| ATOM | 78 | C | HIS | A | 29 | 35.989 | 13.870 | 16.283 | 1.00 | 28.68 | 6 | C |
| ATOM | 79 | O | HIS | A | 29 | 35.781 | 14.076 | 17.474 | 1.00 | 28.88 | 8 | O |
| ATOM | 80 | N | SER | A | 30 | 36.135 | 14.849 | 15.396 | 1.00 | 28.42 | 7 | N |
| ATOM | 81 | CA | SER | A | 30 | 36.122 | 16.241 | 15.810 | 1.00 | 28.05 | 6 | C |
| ATOM | 82 | CB | SER | A | 30 | 36.479 | 17.148 | 14.628 | 1.00 | 28.85 | 6 | C |
| ATOM | 83 | OG | SER | A | 30 | 36.538 | 18.498 | 15.053 | 1.00 | 30.19 | 8 | O |
| ATOM | 84 | C | SER | A | 30 | 34.811 | 16.685 | 16.452 | 1.00 | 27.75 | 6 | C |
| ATOM | 85 | O | SER | A | 30 | 34.812 | 17.298 | 17.521 | 1.00 | 26.57 | 8 | O |
| ATOM | 86 | N | VAL | A | 31 | 33.683 | 16.396 | 15.807 | 1.00 | 27.10 | 7 | N |
| ATOM | 87 | CA | VAL | A | 31 | 32.415 | 16.802 | 16.396 | 1.00 | 26.70 | 6 | C |
| ATOM | 88 | CB | VAL | A | 31 | 31.227 | 16.754 | 15.377 | 1.00 | 27.14 | 6 | C |
| ATOM | 89 | CG1 | VAL | A | 31 | 31.125 | 15.396 | 14.732 | 1.00 | 26.15 | 6 | C |
| ATOM | 90 | CG2 | VAL | A | 31 | 29.904 | 17.116 | 16.063 | 1.00 | 26.78 | 6 | C |
| ATOM | 91 | C | VAL | A | 31 | 32.095 | 15.979 | 17.658 | 1.00 | 26.15 | 6 | C |
| ATOM | 92 | O | VAL | A | 31 | 31.607 | 16.529 | 18.647 | 1.00 | 25.84 | 8 | O |
| ATOM | 93 | N | ASN | A | 32 | 32.375 | 14.677 | 17.632 | 1.00 | 25.70 | 7 | N |
| ATOM | 94 | CA | ASN | A | 32 | 32.050 | 13.827 | 18.789 | 1.00 | 25.94 | 6 | C |
| ATOM | 95 | CB | ASN | A | 32 | 32.251 | 12.348 | 18.486 | 1.00 | 25.11 | 6 | C |
| ATOM | 96 | CG | ASN | A | 32 | 31.242 | 11.800 | 17.473 | 1.00 | 25.06 | 6 | C |
| ATOM | 97 | OD1 | ASN | A | 32 | 30.221 | 12.410 | 17.196 | 1.00 | 25.48 | 8 | O |
| ATOM | 98 | ND2 | ASN | A | 32 | 31.550 | 10.645 | 16.924 | 1.00 | 24.23 | 7 | N |
| ATOM | 99 | C | ASN | A | 32 | 32.875 | 14.188 | 20.022 | 1.00 | 26.29 | 6 | C |
| ATOM | 100 | O | ASN | A | 32 | 32.378 | 14.153 | 21.142 | 1.00 | 26.53 | 8 | O |
| ATOM | 101 | N | ALA | A | 33 | 34.142 | 14.517 | 19.806 | 1.00 | 26.41 | 7 | N |
| ATOM | 102 | CA | ALA | A | 33 | 35.035 | 14.890 | 20.918 | 1.00 | 27.37 | 6 | C |
| ATOM | 103 | CB | ALA | A | 33 | 36.468 | 15.007 | 20.435 | 1.00 | 27.30 | 6 | C |
| ATOM | 104 | C | ALA | A | 33 | 34.595 | 16.187 | 21.584 | 1.00 | 27.63 | 6 | C |
| ATOM | 105 | O | ALA | A | 33 | 34.921 | 16.447 | 22.743 | 1.00 | 27.93 | 8 | O |
| ATOM | 106 | N | SER | A | 34 | 33.834 | 16.994 | 20.858 | 1.00 | 27.83 | 7 | N |
| ATOM | 107 | CA | SER | A | 34 | 33.347 | 18.251 | 21.397 | 1.00 | 28.41 | 6 | C |
| ATOM | 108 | CB | SER | A | 34 | 33.075 | 19.252 | 20.268 | 1.00 | 28.21 | 6 | C |
| ATOM | 109 | OG | SER | A | 34 | 31.807 | 19.031 | 19.670 | 1.00 | 27.66 | 8 | O |
| ATOM | 110 | C | SER | A | 34 | 32.105 | 18.089 | 22.290 | 1.00 | 28.78 | 6 | C |
| ATOM | 111 | O | SER | A | 34 | 31.643 | 19.069 | 22.882 | 1.00 | 28.93 | 8 | O |
| ATOM | 112 | N | LYS | A | 35 | 31.597 | 16.857 | 22.397 | 1.00 | 28.68 | 7 | N |
| ATOM | 113 | CA | LYS | A | 35 | 30.425 | 16.523 | 23.229 | 1.00 | 29.44 | 6 | C |
| ATOM | 114 | CB | LYS | A | 35 | 30.795 | 16.531 | 24.711 | 1.00 | 29.93 | 6 | C |
| ATOM | 115 | CG | LYS | A | 35 | 31.934 | 15.594 | 25.089 | 1.00 | 31.61 | 6 | C |
| ATOM | 116 | CD | LYS | A | 35 | 32.098 | 15.557 | 26.612 | 1.00 | 34.33 | 6 | C |
| ATOM | 117 | CE | LYS | A | 35 | 32.129 | 16.969 | 27.205 | 1.00 | 36.89 | 6 | C |
| ATOM | 118 | NZ | LYS | A | 35 | 32.313 | 16.996 | 28.699 | 1.00 | 39.71 | 7 | N |
| ATOM | 119 | C | LYS | A | 35 | 29.261 | 17.475 | 22.987 | 1.00 | 29.56 | 6 | C |
| ATOM | 120 | O | LYS | A | 35 | 28.822 | 18.180 | 23.894 | 1.00 | 29.19 | 8 | O |
| ATOM | 121 | N | PRO | A | 36 | 28.746 | 17.459 | 21.762 | 1.00 | 29.62 | 7 | N |
| ATOM | 122 | CA | PRO | A | 36 | 27.742 | 18.428 | 21.311 | 1.00 | 29.86 | 6 | C |
| ATOM | 123 | CB | PRO | A | 36 | 27.509 | 18.018 | 19.849 | 1.00 | 29.74 | 6 | C |
| ATOM | 124 | CG | PRO | A | 36 | 27.873 | 16.537 | 19.841 | 1.00 | 29.62 | 6 | C |
| ATOM | 125 | CD | PRO | A | 36 | 29.099 | 16.493 | 20.706 | 1.00 | 29.42 | 6 | C |
| ATOM | 126 | C | PRO | A | 36 | 26.424 | 18.435 | 22.079 | 1.00 | 30.15 | 6 | C |
| ATOM | 127 | O | PRO | A | 36 | 25.743 | 19.461 | 22.046 | 1.00 | 29.59 | 8 | O |
| ATOM | 128 | N | SER | A | 37 | 26.056 | 17.335 | 22.742 | 1.00 | 30.35 | 7 | N |
| ATOM | 129 | CA | SER | A | 37 | 24.796 | 17.310 | 23.482 | 1.00 | 30.73 | 6 | C |
| ATOM | 130 | CB | SER | A | 37 | 24.096 | 15.950 | 23.337 | 1.00 | 30.91 | 6 | C |
| ATOM | 131 | OG | SER | A | 37 | 24.788 | 14.951 | 24.059 | 1.00 | 30.05 | 8 | O |
| ATOM | 132 | C | SER | A | 37 | 24.988 | 17.653 | 24.963 | 1.00 | 31.78 | 6 | C |
| ATOM | 133 | O | SER | A | 37 | 24.028 | 17.746 | 25.717 | 1.00 | 31.53 | 8 | O |
| ATOM | 134 | N | GLU | A | 38 | 26.234 | 17.860 | 25.358 | 1.00 | 32.67 | 7 | N |
| ATOM | 135 | CA | GLU | A | 38 | 26.562 | 18.138 | 26.743 | 1.00 | 34.86 | 6 | C |
| ATOM | 136 | CB | GLU | A | 38 | 27.696 | 17.206 | 27.183 | 1.00 | 34.88 | 6 | C |
| ATOM | 137 | CG | GLU | A | 38 | 27.227 | 15.750 | 27.139 | 1.00 | 37.05 | 6 | C |
| ATOM | 138 | CD | GLU | A | 38 | 28.344 | 14.733 | 26.972 | 1.00 | 40.86 | 6 | C |
| ATOM | 139 | OE1 | GLU | A | 38 | 29.059 | 14.473 | 27.960 | 1.00 | 40.91 | 8 | O |
| ATOM | 140 | OE2 | GLU | A | 38 | 28.496 | 14.175 | 25.852 | 1.00 | 42.71 | 8 | O |
| ATOM | 141 | C | GLU | A | 38 | 26.875 | 19.622 | 26.931 | 1.00 | 35.55 | 6 | C |
| ATOM | 142 | O | GLU | A | 38 | 27.772 | 20.009 | 27.672 | 1.00 | 36.66 | 8 | O |
| ATOM | 143 | N | ARG | A | 39 | 26.091 | 20.442 | 26.244 | 1.00 | 36.48 | 7 | N |
| ATOM | 144 | CA | ARG | A | 39 | 26.224 | 21.887 | 26.286 | 1.00 | 37.25 | 6 | C |
| ATOM | 145 | CB | ARG | A | 39 | 26.169 | 22.456 | 24.865 | 1.00 | 37.41 | 6 | C |
| ATOM | 146 | CG | ARG | A | 39 | 27.186 | 21.845 | 23.903 | 1.00 | 38.11 | 6 | C |
| ATOM | 147 | CD | ARG | A | 39 | 28.580 | 22.457 | 24.002 | 1.00 | 38.15 | 6 | C |
| ATOM | 148 | NE | ARG | A | 39 | 29.559 | 21.725 | 23.204 | 1.00 | 38.73 | 7 | N |
| ATOM | 149 | CZ | ARG | A | 39 | 29.706 | 21.866 | 21.893 | 1.00 | 37.81 | 6 | C |
| ATOM | 150 | NH1 | ARG | A | 39 | 28.941 | 22.718 | 21.228 | 1.00 | 37.87 | 7 | N |
| ATOM | 151 | NH2 | ARG | A | 39 | 30.616 | 21.153 | 21.249 | 1.00 | 37.30 | 7 | N |
| ATOM | 152 | C | ARG | A | 39 | 25.058 | 22.433 | 27.079 | 1.00 | 37.01 | 6 | C |
| ATOM | 153 | O | ARG | A | 39 | 24.022 | 21.783 | 27.198 | 1.00 | 37.60 | 8 | O |
| ATOM | 154 | N | GLY | A | 40 | 25.207 | 23.636 | 27.607 | 1.00 | 36.91 | 7 | N |
| ATOM | 155 | CA | GLY | A | 40 | 24.123 | 24.232 | 28.365 | 1.00 | 36.46 | 6 | C |
| ATOM | 156 | C | GLY | A | 40 | 22.905 | 24.597 | 27.533 | 1.00 | 35.96 | 6 | C |
| ATOM | 157 | O | GLY | A | 40 | 21.769 | 24.456 | 27.975 | 1.00 | 37.22 | 8 | O |
| ATOM | 158 | N | LEU | A | 41 | 23.134 | 25.097 | 26.331 | 1.00 | 34.84 | 7 | N |
| ATOM | 159 | CA | LEU | A | 41 | 22.045 | 25.476 | 25.452 | 1.00 | 33.51 | 6 | C |
| ATOM | 160 | CB | LEU | A | 41 | 21.947 | 27.000 | 25.353 | 1.00 | 33.41 | 6 | C |
| ATOM | 161 | CG | LEU | A | 41 | 20.995 | 27.589 | 24.315 | 1.00 | 33.40 | 6 | C |
| ATOM | 162 | CD1 | LEU | A | 41 | 19.561 | 27.317 | 24.719 | 1.00 | 33.71 | 6 | C |
| ATOM | 163 | CD2 | LEU | A | 41 | 21.232 | 29.095 | 24.155 | 1.00 | 33.45 | 6 | C |
| ATOM | 164 | C | LEU | A | 41 | 22.353 | 24.903 | 24.085 | 1.00 | 33.07 | 6 | C |
| ATOM | 165 | O | LEU | A | 41 | 23.431 | 25.131 | 23.548 | 1.00 | 33.54 | 8 | O |
| ATOM | 166 | N | VAL | A | 42 | 21.419 | 24.146 | 23.532 | 1.00 | 32.14 | 7 | N |
| ATOM | 167 | CA | VAL | A | 42 | 21.617 | 23.570 | 22.208 | 1.00 | 31.38 | 6 | C |
| ATOM | 168 | CB | VAL | A | 42 | 21.141 | 22.101 | 22.170 | 1.00 | 31.56 | 6 | C |
| ATOM | 169 | CG1 | VAL | A | 42 | 21.051 | 21.591 | 20.724 | 1.00 | 31.57 | 6 | C |
| ATOM | 170 | CG2 | VAL | A | 42 | 22.086 | 21.241 | 22.991 | 1.00 | 30.77 | 6 | C |
| ATOM | 171 | C | VAL | A | 42 | 20.912 | 24.406 | 21.148 | 1.00 | 31.02 | 6 | C |
| ATOM | 172 | O | VAL | A | 42 | 19.771 | 24.820 | 21.340 | 1.00 | 31.11 | 8 | O |
| ATOM | 173 | N | ARG | A | 43 | 21.619 | 24.692 | 20.055 | 1.00 | 30.35 | 7 | N |
| ATOM | 174 | CA | ARG | A | 43 | 21.061 | 25.422 | 18.927 | 1.00 | 30.73 | 6 | C |
| ATOM | 175 | CB | ARG | A | 43 | 21.636 | 26.843 | 18.839 | 1.00 | 30.57 | 6 | C |
| ATOM | 176 | CG | ARG | A | 43 | 21.148 | 27.756 | 19.974 | 1.00 | 32.57 | 6 | C |
| ATOM | 177 | CD | ARG | A | 43 | 21.173 | 29.237 | 19.630 | 1.00 | 33.26 | 6 | C |
| ATOM | 178 | NE | ARG | A | 43 | 22.519 | 29.784 | 19.645 | 1.00 | 33.72 | 7 | N |
| ATOM | 179 | CZ | ARG | A | 43 | 22.929 | 30.792 | 18.880 | 1.00 | 33.34 | 6 | C |
| ATOM | 180 | NH1 | ARG | A | 43 | 22.106 | 31.358 | 18.006 | 1.00 | 34.75 | 7 | N |
| ATOM | 181 | NH2 | ARG | A | 43 | 24.169 | 31.228 | 18.986 | 1.00 | 34.75 | 7 | N |
| ATOM | 182 | C | ARG | A | 43 | 21.325 | 24.641 | 17.640 | 1.00 | 30.19 | 6 | C |
| ATOM | 183 | O | ARG | A | 43 | 22.000 | 25.114 | 16.731 | 1.00 | 30.53 | 8 | O |
| ATOM | 184 | N | GLN | A | 44 | 20.794 | 23.427 | 17.595 | 1.00 | 30.12 | 7 | N |
| ATOM | 185 | CA | GLN | A | 44 | 20.953 | 22.529 | 16.453 | 1.00 | 30.34 | 6 | C |
| ATOM | 186 | CB | GLN | A | 44 | 20.082 | 21.289 | 16.681 | 1.00 | 30.51 | 6 | C |
| ATOM | 187 | CG | GLN | A | 44 | 20.483 | 20.058 | 15.907 | 1.00 | 32.20 | 6 | C |
| ATOM | 188 | CD | GLN | A | 44 | 19.725 | 18.832 | 16.380 | 1.00 | 33.37 | 6 | C |
| ATOM | 189 | OE1 | GLN | A | 44 | 19.786 | 18.488 | 17.549 | 1.00 | 34.97 | 8 | O |
| ATOM | 190 | NE2 | GLN | A | 44 | 19.001 | 18.184 | 15.476 | 1.00 | 34.78 | 7 | N |
| ATOM | 191 | C | GLN | A | 44 | 20.571 | 23.181 | 15.132 | 1.00 | 29.89 | 6 | C |
| ATOM | 192 | O | GLN | A | 44 | 21.191 | 22.925 | 14.097 | 1.00 | 29.81 | 8 | O |
| ATOM | 193 | N | ALA | A | 45 | 19.543 | 24.022 | 15.155 | 1.00 | 29.90 | 7 | N |
| ATOM | 194 | CA | ALA | A | 45 | 19.060 | 24.636 | 13.920 | 1.00 | 30.12 | 6 | C |
| ATOM | 195 | CB | ALA | A | 45 | 17.754 | 25.393 | 14.155 | 1.00 | 30.53 | 6 | C |
| ATOM | 196 | C | ALA | A | 45 | 20.095 | 25.532 | 13.259 | 1.00 | 30.05 | 6 | C |
| ATOM | 197 | O | ALA | A | 45 | 20.044 | 25.762 | 12.054 | 1.00 | 29.93 | 8 | O |
| ATOM | 198 | N | GLU | A | 46 | 21.051 | 26.018 | 14.039 | 1.00 | 29.96 | 7 | N |
| ATOM | 199 | CA | GLU | A | 46 | 22.078 | 26.895 | 13.501 | 1.00 | 30.20 | 6 | C |
| ATOM | 200 | CB | GLU | A | 46 | 22.767 | 27.675 | 14.628 | 1.00 | 30.43 | 6 | C |
| ATOM | 201 | CG | GLU | A | 46 | 21.879 | 28.715 | 15.287 | 1.00 | 32.59 | 6 | C |
| ATOM | 202 | CD | GLU | A | 46 | 21.397 | 29.779 | 14.324 | 1.00 | 33.11 | 6 | C |
| ATOM | 203 | OE1 | GLU | A | 46 | 22.124 | 30.091 | 13.354 | 1.00 | 34.58 | 8 | O |
| ATOM | 204 | OE2 | GLU | A | 46 | 20.290 | 30.319 | 14.537 | 1.00 | 35.13 | 8 | O |
| ATOM | 205 | C | GLU | A | 46 | 23.112 | 26.121 | 12.687 | 1.00 | 29.91 | 6 | C |
| ATOM | 206 | O | GLU | A | 46 | 23.984 | 26.717 | 12.053 | 1.00 | 29.50 | 8 | O |
| ATOM | 207 | N | ALA | A | 47 | 23.007 | 24.795 | 12.699 | 1.00 | 29.19 | 7 | N |
| ATOM | 208 | CA | ALA | A | 47 | 23.948 | 23.958 | 11.957 | 1.00 | 28.42 | 6 | C |
| ATOM | 209 | CB | ALA | A | 47 | 24.384 | 22.768 | 12.804 | 1.00 | 28.21 | 6 | C |
| ATOM | 210 | C | ALA | A | 47 | 23.339 | 23.473 | 10.641 | 1.00 | 28.77 | 6 | C |
| ATOM | 211 | O | ALA | A | 47 | 24.010 | 22.818 | 9.843 | 1.00 | 27.99 | 8 | O |
| ATOM | 212 | N | GLU | A | 48 | 22.071 | 23.802 | 10.423 | 1.00 | 28.97 | 7 | N |
| ATOM | 213 | CA | GLU | A | 48 | 21.373 | 23.409 | 9.196 | 1.00 | 29.55 | 6 | C |
| ATOM | 214 | CB | GLU | A | 48 | 19.886 | 23.769 | 9.292 | 1.00 | 29.77 | 6 | C |
| ATOM | 215 | CG | GLU | A | 48 | 19.116 | 23.003 | 10.360 | 1.00 | 31.36 | 6 | C |
| ATOM | 216 | CD | GLU | A | 48 | 17.644 | 23.379 | 10.405 | 1.00 | 33.36 | 6 | C |
| ATOM | 217 | OE1 | GLU | A | 48 | 17.200 | 24.140 | 9.524 | 1.00 | 33.45 | 8 | O |
| ATOM | 218 | OE2 | GLU | A | 48 | 16.930 | 22.917 | 11.324 | 1.00 | 34.99 | 8 | O |
| ATOM | 219 | C | GLU | A | 48 | 21.975 | 24.062 | 7.949 | 1.00 | 29.78 | 6 | C |
| ATOM | 220 | O | GLU | A | 48 | 22.231 | 25.265 | 7.921 | 1.00 | 28.44 | 8 | O |
| ATOM | 221 | N | ASP | A | 49 | 22.188 | 23.260 | 6.911 | 1.00 | 30.40 | 7 | N |
| ATOM | 222 | CA | ASP | A | 49 | 22.754 | 23.765 | 5.666 | 1.00 | 31.48 | 6 | C |
| ATOM | 223 | CB | ASP | A | 49 | 24.268 | 23.563 | 5.663 | 1.00 | 32.13 | 6 | C |
| ATOM | 224 | CG | ASP | A | 49 | 24.990 | 24.508 | 4.716 | 1.00 | 34.05 | 6 | C |
| ATOM | 225 | OD1 | ASP | A | 49 | 24.342 | 25.064 | 3.807 | 1.00 | 35.90 | 8 | O |
| ATOM | 226 | OD2 | ASP | A | 49 | 26.215 | 24.752 | 4.813 | 1.00 | 36.69 | 8 | O |
| ATOM | 227 | C | ASP | A | 49 | 22.112 | 22.973 | 4.531 | 1.00 | 31.75 | 6 | C |
| ATOM | 228 | O | ASP | A | 49 | 22.643 | 21.949 | 4.106 | 1.00 | 31.48 | 8 | O |
| ATOM | 229 | N | PRO | A | 50 | 20.966 | 23.445 | 4.054 | 1.00 | 32.06 | 7 | N |
| ATOM | 230 | CA | PRO | A | 50 | 20.224 | 22.748 | 2.994 | 1.00 | 32.74 | 6 | C |
| ATOM | 231 | CB | PRO | A | 50 | 18.970 | 23.615 | 2.810 | 1.00 | 33.04 | 6 | C |
| ATOM | 232 | CG | PRO | A | 50 | 18.897 | 24.474 | 4.020 | 1.00 | 32.80 | 6 | C |
| ATOM | 233 | CD | PRO | A | 50 | 20.301 | 24.689 | 4.475 | 1.00 | 32.50 | 6 | C |
| ATOM | 234 | C | PRO | A | 50 | 21.003 | 22.673 | 1.689 | 1.00 | 33.09 | 6 | C |
| ATOM | 235 | O | PRO | A | 50 | 20.700 | 21.823 | 0.839 | 1.00 | 33.20 | 8 | O |
| ATOM | 236 | N | ALA | A | 51 | 21.994 | 23.540 | 1.519 | 1.00 | 33.00 | 7 | N |
| ATOM | 237 | CA | ALA | A | 51 | 22.807 | 23.506 | 0.305 | 1.00 | 32.95 | 6 | C |
| ATOM | 238 | CB | ALA | A | 51 | 23.614 | 24.780 | 0.159 | 1.00 | 33.32 | 6 | C |
| ATOM | 239 | C | ALA | A | 51 | 23.728 | 22.277 | 0.274 | 1.00 | 32.90 | 6 | C |
| ATOM | 240 | O | ALA | A | 51 | 24.358 | 21.972 | −0.748 | 1.00 | 32.85 | 8 | O |
| ATOM | 241 | N | CYS | A | 52 | 23.791 | 21.571 | 1.395 | 1.00 | 31.55 | 7 | N |
| ATOM | 242 | CA | CYS | A | 52 | 24.631 | 20.386 | 1.495 | 1.00 | 31.19 | 6 | C |
| ATOM | 243 | CB | CYS | A | 52 | 25.420 | 20.413 | 2.799 | 1.00 | 31.18 | 6 | C |
| ATOM | 244 | SG | CYS | A | 52 | 26.601 | 21.780 | 2.860 | 1.00 | 36.09 | 16 | S |
| ATOM | 245 | C | CYS | A | 52 | 23.861 | 19.074 | 1.371 | 1.00 | 29.52 | 6 | C |
| ATOM | 246 | O | CYS | A | 52 | 24.444 | 18.009 | 1.518 | 1.00 | 29.12 | 8 | O |
| ATOM | 247 | N | ILE | A | 53 | 22.562 | 19.150 | 1.107 | 1.00 | 28.43 | 7 | N |
| ATOM | 248 | CA | ILE | A | 53 | 21.753 | 17.939 | 0.942 | 1.00 | 28.06 | 6 | C |
| ATOM | 249 | CB | ILE | A | 53 | 20.289 | 18.316 | 0.567 | 1.00 | 28.23 | 6 | C |
| ATOM | 250 | CG1 | ILE | A | 53 | 19.661 | 19.129 | 1.708 | 1.00 | 30.03 | 6 | C |
| ATOM | 251 | CD1 | ILE | A | 53 | 18.283 | 19.739 | 1.391 | 1.00 | 32.54 | 6 | C |
| ATOM | 252 | CG2 | ILE | A | 53 | 19.448 | 17.078 | 0.345 | 1.00 | 29.37 | 6 | C |
| ATOM | 253 | C | ILE | A | 53 | 22.429 | 17.048 | −0.109 | 1.00 | 27.44 | 6 | C |
| ATOM | 254 | O | ILE | A | 53 | 22.935 | 17.550 | −1.114 | 1.00 | 26.15 | 8 | O |
| ATOM | 255 | N | PRO | A | 54 | 22.469 | 15.740 | 0.133 | 1.00 | 27.31 | 7 | N |
| ATOM | 256 | CA | PRO | A | 54 | 23.141 | 14.815 | −0.784 | 1.00 | 27.58 | 6 | C |
| ATOM | 257 | CB | PRO | A | 54 | 23.057 | 13.458 | −0.065 | 1.00 | 27.83 | 6 | C |
| ATOM | 258 | CG | PRO | A | 54 | 22.513 | 13.719 | 1.293 | 1.00 | 28.07 | 6 | C |
| ATOM | 259 | CD | PRO | A | 54 | 21.853 | 15.052 | 1.281 | 1.00 | 27.24 | 6 | C |
| ATOM | 260 | C | PRO | A | 54 | 22.413 | 14.683 | −2.117 | 1.00 | 27.78 | 6 | C |
| ATOM | 261 | O | PRO | A | 54 | 21.196 | 14.891 | −2.189 | 1.00 | 27.38 | 8 | O |
| ATOM | 262 | N | ILE | A | 55 | 23.163 | 14.332 | −3.154 | 1.00 | 27.67 | 7 | N |
| ATOM | 263 | CA | ILE | A | 55 | 22.585 | 14.048 | −4.454 | 1.00 | 28.32 | 6 | C |
| ATOM | 264 | CB | ILE | A | 55 | 23.666 | 14.179 | −5.548 | 1.00 | 28.85 | 6 | C |
| ATOM | 265 | CG1 | ILE | A | 55 | 24.293 | 15.579 | −5.494 | 1.00 | 30.95 | 6 | C |
| ATOM | 266 | CD1 | ILE | A | 55 | 25.740 | 15.648 | −5.965 | 1.00 | 33.86 | 6 | C |
| ATOM | 267 | CG2 | ILE | A | 55 | 23.054 | 13.925 | −6.929 | 1.00 | 30.22 | 6 | C |
| ATOM | 268 | C | ILE | A | 55 | 21.983 | 12.635 | −4.455 | 1.00 | 27.58 | 6 | C |
| ATOM | 269 | O | ILE | A | 55 | 20.922 | 12.400 | −5.017 | 1.00 | 27.41 | 8 | O |
| ATOM | 270 | N | PHE | A | 56 | 22.660 | 11.702 | −3.790 | 1.00 | 26.32 | 7 | N |
| ATOM | 271 | CA | PHE | A | 56 | 22.237 | 10.314 | −3.766 | 1.00 | 25.72 | 6 | C |
| ATOM | 272 | CB | PHE | A | 56 | 23.218 | 9.453 | −4.581 | 1.00 | 25.89 | 6 | C |
| ATOM | 273 | CG | PHE | A | 56 | 23.324 | 9.836 | −6.034 | 1.00 | 27.67 | 6 | C |
| ATOM | 274 | CD1 | PHE | A | 56 | 24.429 | 10.528 | −6.498 | 1.00 | 27.36 | 6 | C |
| ATOM | 275 | CE1 | PHE | A | 56 | 24.546 | 10.875 | −7.834 | 1.00 | 28.92 | 6 | C |
| ATOM | 276 | CZ | PHE | A | 56 | 23.556 | 10.552 | −8.719 | 1.00 | 29.32 | 6 | C |
| ATOM | 277 | CE2 | PHE | A | 56 | 22.437 | 9.856 | −8.280 | 1.00 | 30.40 | 6 | C |
| ATOM | 278 | CD2 | PHE | A | 56 | 22.327 | 9.496 | −6.934 | 1.00 | 29.35 | 6 | C |
| ATOM | 279 | C | PHE | A | 56 | 22.226 | 9.718 | −2.361 | 1.00 | 24.91 | 6 | C |
| ATOM | 280 | O | PHE | A | 56 | 23.036 | 10.085 | −1.513 | 1.00 | 23.97 | 8 | O |
| ATOM | 281 | N | TRP | A | 57 | 21.312 | 8.781 | −2.142 | 1.00 | 24.36 | 7 | N |
| ATOM | 282 | CA | TRP | A | 57 | 21.297 | 7.945 | −0.942 | 1.00 | 24.65 | 6 | C |
| ATOM | 283 | CB | TRP | A | 57 | 20.622 | 8.641 | 0.260 | 1.00 | 24.01 | 6 | C |
| ATOM | 284 | CG | TRP | A | 57 | 19.175 | 9.036 | 0.010 | 1.00 | 25.03 | 6 | C |
| ATOM | 285 | CD1 | TRP | A | 57 | 18.053 | 8.270 | 0.217 | 1.00 | 24.50 | 6 | C |
| ATOM | 286 | NE1 | TRP | A | 57 | 16.924 | 8.976 | −0.142 | 1.00 | 23.27 | 7 | N |
| ATOM | 287 | CE2 | TRP | A | 57 | 17.298 | 10.223 | −0.578 | 1.00 | 25.29 | 6 | C |
| ATOM | 288 | CD2 | TRP | A | 57 | 18.705 | 10.298 | −0.491 | 1.00 | 24.40 | 6 | C |
| ATOM | 289 | CE3 | TRP | A | 57 | 19.337 | 11.486 | −0.885 | 1.00 | 26.85 | 6 | C |
| ATOM | 290 | CZ3 | TRP | A | 57 | 18.551 | 12.544 | −1.342 | 1.00 | 28.68 | 6 | C |
| ATOM | 291 | CH2 | TRP | A | 57 | 17.162 | 12.434 | −1.409 | 1.00 | 27.81 | 6 | C |
| ATOM | 292 | CZ2 | TRP | A | 57 | 16.516 | 11.289 | −1.029 | 1.00 | 26.93 | 6 | C |
| ATOM | 293 | C | TRP | A | 57 | 20.572 | 6.649 | −1.319 | 1.00 | 24.73 | 6 | C |
| ATOM | 294 | O | TRP | A | 57 | 19.945 | 6.577 | −2.386 | 1.00 | 24.59 | 8 | O |
| ATOM | 295 | N | VAL | A | 58 | 20.684 | 5.630 | −0.476 | 1.00 | 24.55 | 7 | N |
| ATOM | 296 | CA | VAL | A | 58 | 19.994 | 4.364 | −0.702 | 1.00 | 24.83 | 6 | C |
| ATOM | 297 | CB | VAL | A | 58 | 20.741 | 3.191 | −0.036 | 1.00 | 24.93 | 6 | C |
| ATOM | 298 | CG1 | VAL | A | 58 | 19.939 | 1.887 | −0.162 | 1.00 | 24.14 | 6 | C |
| ATOM | 299 | CG2 | VAL | A | 58 | 22.109 | 3.016 | −0.677 | 1.00 | 25.68 | 6 | C |
| ATOM | 300 | C | VAL | A | 58 | 18.544 | 4.447 | −0.204 | 1.00 | 25.64 | 6 | C |
| ATOM | 301 | O | VAL | A | 58 | 18.296 | 4.723 | 0.976 | 1.00 | 25.76 | 8 | O |
| ATOM | 302 | N | SER | A | 59 | 17.597 | 4.220 | −1.119 | 1.00 | 25.84 | 7 | N |
| ATOM | 303 | CA | SER | A | 59 | 16.170 | 4.341 | −0.834 | 1.00 | 26.79 | 6 | C |
| ATOM | 304 | CB | SER | A | 59 | 15.449 | 4.907 | −2.064 | 1.00 | 27.22 | 6 | C |
| ATOM | 305 | OG | SER | A | 59 | 15.274 | 6.298 | −1.930 | 1.00 | 32.34 | 8 | O |
| ATOM | 306 | C | SER | A | 59 | 15.513 | 3.028 | −0.437 | 1.00 | 26.12 | 6 | C |
| ATOM | 307 | O | SER | A | 59 | 14.527 | 3.018 | 0.314 | 1.00 | 25.45 | 8 | O |
| ATOM | 308 | N | LYS | A | 60 | 16.042 | 1.924 | −0.965 | 1.00 | 25.71 | 7 | N |
| ATOM | 309 | CA | LYS | A | 60 | 15.524 | 0.592 | −0.674 | 1.00 | 25.00 | 6 | C |
| ATOM | 310 | CB | LYS | A | 60 | 14.420 | 0.183 | −1.665 | 1.00 | 25.95 | 6 | C |
| ATOM | 311 | CG | LYS | A | 60 | 13.282 | 1.185 | −1.857 | 1.00 | 25.89 | 6 | C |
| ATOM | 312 | CD | LYS | A | 60 | 12.358 | 0.774 | −3.030 | 1.00 | 27.60 | 6 | C |
| ATOM | 313 | CE | LYS | A | 60 | 11.199 | 1.774 | −3.198 | 1.00 | 27.10 | 6 | C |
| ATOM | 314 | NZ | LYS | A | 60 | 10.221 | 1.300 | −4.234 | 1.00 | 27.31 | 7 | N |
| ATOM | 315 | C | LYS | A | 60 | 16.697 | −0.374 | −0.821 | 1.00 | 24.95 | 6 | C |
| ATOM | 316 | O | LYS | A | 60 | 17.665 | −0.065 | −1.513 | 1.00 | 24.15 | 8 | O |
| ATOM | 317 | N | TRP | A | 61 | 16.625 | −1.510 | −0.148 | 1.00 | 24.47 | 7 | N |
| ATOM | 318 | CA | TRP | A | 61 | 17.656 | −2.541 | −0.280 | 1.00 | 25.47 | 6 | C |
| ATOM | 319 | CB | TRP | A | 61 | 18.899 | −2.210 | 0.566 | 1.00 | 25.28 | 6 | C |
| ATOM | 320 | CG | TRP | A | 61 | 18.610 | −2.062 | 2.003 | 1.00 | 25.61 | 6 | C |
| ATOM | 321 | CD1 | TRP | A | 61 | 18.356 | −0.900 | 2.677 | 1.00 | 25.43 | 6 | C |
| ATOM | 322 | NE1 | TRP | A | 61 | 18.136 | −1.169 | 4.008 | 1.00 | 25.56 | 7 | N |
| ATOM | 323 | CE2 | TRP | A | 61 | 18.229 | −2.520 | 4.213 | 1.00 | 26.39 | 6 | C |
| ATOM | 324 | CD2 | TRP | A | 61 | 18.538 | −3.113 | 2.974 | 1.00 | 26.25 | 6 | C |
| ATOM | 325 | CE3 | TRP | A | 61 | 18.698 | −4.504 | 2.918 | 1.00 | 26.30 | 6 | C |
| ATOM | 326 | CZ3 | TRP | A | 61 | 18.543 | −5.245 | 4.080 | 1.00 | 27.94 | 6 | C |
| ATOM | 327 | CH2 | TRP | A | 61 | 18.229 | −4.624 | 5.299 | 1.00 | 27.03 | 6 | C |
| ATOM | 328 | CZ2 | TRP | A | 61 | 18.077 | −3.267 | 5.388 | 1.00 | 26.38 | 6 | C |
| ATOM | 329 | C | TRP | A | 61 | 17.102 | −3.920 | 0.083 | 1.00 | 26.16 | 6 | C |
| ATOM | 330 | O | TRP | A | 61 | 16.158 | −4.037 | 0.871 | 1.00 | 25.82 | 8 | O |
| ATOM | 331 | N | VAL | A | 62 | 17.709 | −4.958 | −0.487 | 1.00 | 27.04 | 7 | N |
| ATOM | 332 | CA | VAL | A | 62 | 17.326 | −6.346 | −0.236 | 1.00 | 28.50 | 6 | C |
| ATOM | 333 | CB | VAL | A | 62 | 16.530 | −6.937 | −1.428 | 1.00 | 28.90 | 6 | C |
| ATOM | 334 | CG1 | VAL | A | 62 | 16.036 | −8.339 | −1.100 | 1.00 | 30.44 | 6 | C |
| ATOM | 335 | CG2 | VAL | A | 62 | 15.361 | −6.042 | −1.808 | 1.00 | 29.34 | 6 | C |
| ATOM | 336 | C | VAL | A | 62 | 18.600 | −7.169 | −0.076 | 1.00 | 29.14 | 6 | C |
| ATOM | 337 | O | VAL | A | 62 | 19.449 | −7.171 | −0.962 | 1.00 | 27.94 | 8 | O |
| ATOM | 338 | N | ASP | A | 63 | 18.726 | −7.869 | 1.048 | 1.00 | 30.63 | 7 | N |
| ATOM | 339 | CA | ASP | A | 63 | 19.911 | −8.672 | 1.335 | 1.00 | 31.95 | 6 | C |
| ATOM | 340 | CB | ASP | A | 63 | 20.241 | −8.584 | 2.824 | 1.00 | 31.90 | 6 | C |
| ATOM | 341 | CG | ASP | A | 63 | 21.484 | −9.378 | 3.215 | 1.00 | 32.72 | 6 | C |
| ATOM | 342 | OD1 | ASP | A | 63 | 22.047 | −10.133 | 2.383 | 1.00 | 33.46 | 8 | O |
| ATOM | 343 | OD2 | ASP | A | 63 | 21.962 | −9.306 | 4.361 | 1.00 | 31.63 | 8 | O |
| ATOM | 344 | C | ASP | A | 63 | 19.736 | −10.130 | 0.898 | 1.00 | 33.18 | 6 | C |
| ATOM | 345 | O | ASP | A | 63 | 19.187 | −10.958 | 1.632 | 1.00 | 33.29 | 8 | O |
| ATOM | 346 | N | TYR | A | 64 | 20.206 | −10.435 | −0.302 | 1.00 | 34.24 | 7 | N |
| ATOM | 347 | CA | TYR | A | 64 | 20.151 | −11.797 | −0.822 | 1.00 | 36.03 | 6 | C |
| ATOM | 348 | CB | TYR | A | 64 | 19.507 | −11.794 | −2.203 | 1.00 | 36.45 | 6 | C |
| ATOM | 349 | CG | TYR | A | 64 | 18.589 | −12.965 | −2.465 | 1.00 | 41.00 | 6 | C |
| ATOM | 350 | CD1 | TYR | A | 64 | 17.298 | −12.767 | −2.940 | 1.00 | 44.13 | 6 | C |
| ATOM | 351 | CE1 | TYR | A | 64 | 16.452 | −13.837 | −3.179 | 1.00 | 46.58 | 6 | C |
| ATOM | 352 | CZ | TYR | A | 64 | 16.898 | −15.125 | −2.943 | 1.00 | 47.54 | 6 | C |
| ATOM | 353 | OH | TYR | A | 64 | 16.068 | −16.200 | −3.177 | 1.00 | 50.35 | 8 | O |
| ATOM | 354 | CE2 | TYR | A | 64 | 18.175 | −15.346 | −2.471 | 1.00 | 46.59 | 6 | C |
| ATOM | 355 | CD2 | TYR | A | 64 | 19.011 | −14.270 | −2.233 | 1.00 | 44.35 | 6 | C |
| ATOM | 356 | C | TYR | A | 64 | 21.575 | −12.330 | −0.902 | 1.00 | 35.84 | 6 | C |
| ATOM | 357 | O | TYR | A | 64 | 21.925 | −13.065 | −1.823 | 1.00 | 35.73 | 8 | O |
| ATOM | 358 | N | SER | A | 65 | 22.398 | −11.950 | 0.070 | 1.00 | 36.70 | 7 | N |
| ATOM | 359 | CA | SER | A | 65 | 23.818 | −12.299 | 0.047 | 1.00 | 37.40 | 6 | C |
| ATOM | 360 | CB | SER | A | 65 | 24.627 | −11.399 | 0.979 | 1.00 | 37.31 | 6 | C |
| ATOM | 361 | OG | SER | A | 65 | 24.385 | −11.718 | 2.333 | 1.00 | 37.43 | 8 | O |
| ATOM | 362 | C | SER | A | 65 | 24.063 | −13.766 | 0.369 | 1.00 | 38.51 | 6 | C |
| ATOM | 363 | O | SER | A | 65 | 25.198 | −14.229 | 0.368 | 1.00 | 38.36 | 8 | O |
| ATOM | 364 | N | ASP | A | 66 | 22.979 | −14.478 | 0.649 | 1.00 | 39.68 | 7 | N |
| ATOM | 365 | CA | ASP | A | 66 | 23.006 | −15.906 | 0.892 | 1.00 | 40.94 | 6 | C |
| ATOM | 366 | CB | ASP | A | 66 | 21.603 | −16.348 | 1.317 | 1.00 | 41.90 | 6 | C |
| ATOM | 367 | CG | ASP | A | 66 | 21.621 | −17.525 | 2.252 | 1.00 | 45.04 | 6 | C |
| ATOM | 368 | OD1 | ASP | A | 66 | 22.727 | −17.914 | 2.693 | 1.00 | 49.21 | 8 | O |
| ATOM | 369 | OD2 | ASP | A | 66 | 20.575 | −18.128 | 2.603 | 1.00 | 48.82 | 8 | O |
| ATOM | 370 | C | ASP | A | 66 | 23.349 | −16.621 | −0.403 | 1.00 | 40.56 | 6 | C |
| ATOM | 371 | O | ASP | A | 66 | 23.967 | −17.700 | −0.396 | 1.00 | 40.67 | 8 | O |
| ATOM | 372 | N | LYS | A | 67 | 22.945 | −16.018 | −1.518 | 1.00 | 39.66 | 7 | N |
| ATOM | 373 | CA | LYS | A | 67 | 23.078 | −16.670 | −2.819 | 1.00 | 38.94 | 6 | C |
| ATOM | 374 | CB | LYS | A | 67 | 21.744 | −17.344 | −3.183 | 1.00 | 39.57 | 6 | C |
| ATOM | 375 | CG | LYS | A | 67 | 21.368 | −18.486 | −2.245 | 1.00 | 41.78 | 6 | C |
| ATOM | 376 | CD | LYS | A | 67 | 19.921 | −18.941 | −2.419 | 1.00 | 45.87 | 6 | C |
| ATOM | 377 | CE | LYS | A | 67 | 19.497 | −19.841 | −1.248 | 1.00 | 48.20 | 6 | C |
| ATOM | 378 | NZ | LYS | A | 67 | 18.171 | −20.498 | −1.466 | 1.00 | 49.72 | 7 | N |
| ATOM | 379 | C | LYS | A | 67 | 23.540 | −15.783 | −3.979 | 1.00 | 37.58 | 6 | C |
| ATOM | 380 | O | LYS | A | 67 | 24.265 | −16.250 | −4.857 | 1.00 | 37.13 | 8 | O |
| ATOM | 381 | N | TYR | A | 68 | 23.132 | −14.514 | −3.984 | 1.00 | 35.65 | 7 | N |
| ATOM | 382 | CA | TYR | A | 68 | 23.457 | −13.624 | −5.097 | 1.00 | 34.39 | 6 | C |
| ATOM | 383 | CB | TYR | A | 68 | 22.183 | −13.231 | −5.841 | 1.00 | 34.80 | 6 | C |
| ATOM | 384 | CG | TYR | A | 68 | 21.317 | −14.414 | −6.216 | 1.00 | 37.04 | 6 | C |
| ATOM | 385 | CD1 | TYR | A | 68 | 20.096 | −14.625 | −5.593 | 1.00 | 39.02 | 6 | C |
| ATOM | 386 | CE1 | TYR | A | 68 | 19.295 | −15.708 | −5.926 | 1.00 | 40.75 | 6 | C |
| ATOM | 387 | CZ | TYR | A | 68 | 19.710 | −16.596 | −6.895 | 1.00 | 41.85 | 6 | C |
| ATOM | 388 | OH | TYR | A | 68 | 18.897 | −17.663 | −7.223 | 1.00 | 44.07 | 8 | O |
| ATOM | 389 | CE2 | TYR | A | 68 | 20.922 | −16.411 | −7.538 | 1.00 | 40.85 | 6 | C |
| ATOM | 390 | CD2 | TYR | A | 68 | 21.723 | −15.322 | −7.192 | 1.00 | 38.99 | 6 | C |
| ATOM | 391 | C | TYR | A | 68 | 24.236 | −12.365 | −4.720 | 1.00 | 32.93 | 6 | C |
| ATOM | 392 | O | TYR | A | 68 | 25.242 | −12.032 | −5.354 | 1.00 | 31.97 | 8 | O |
| ATOM | 393 | N | GLY | A | 69 | 23.761 | −11.655 | −3.705 | 1.00 | 31.18 | 7 | N |
| ATOM | 394 | CA | GLY | A | 69 | 24.428 | −10.437 | −3.289 | 1.00 | 29.97 | 6 | C |
| ATOM | 395 | C | GLY | A | 69 | 23.447 | −9.452 | −2.675 | 1.00 | 29.19 | 6 | C |
| ATOM | 396 | O | GLY | A | 69 | 22.369 | −9.840 | −2.229 | 1.00 | 28.66 | 8 | O |
| ATOM | 397 | N | LEU | A | 70 | 23.831 | −8.182 | −2.652 | 1.00 | 28.13 | 7 | N |
| ATOM | 398 | CA | LEU | A | 70 | 22.985 | −7.148 | −2.089 | 1.00 | 27.85 | 6 | C |
| ATOM | 399 | CB | LEU | A | 70 | 23.752 | −6.354 | −1.044 | 1.00 | 27.96 | 6 | C |
| ATOM | 400 | CG | LEU | A | 70 | 22.788 | −5.400 | −0.333 | 1.00 | 29.94 | 6 | C |
| ATOM | 401 | CD1 | LEU | A | 70 | 22.772 | −5.653 | 1.159 | 1.00 | 29.95 | 6 | C |
| ATOM | 402 | CD2 | LEU | A | 70 | 23.013 | −3.946 | −0.700 | 1.00 | 30.21 | 6 | C |
| ATOM | 403 | C | LEU | A | 70 | 22.467 | −6.215 | −3.163 | 1.00 | 27.20 | 6 | C |
| ATOM | 404 | O | LEU | A | 70 | 23.244 | −5.529 | −3.811 | 1.00 | 27.61 | 8 | O |
| ATOM | 405 | N | GLY | A | 71 | 21.148 | −6.185 | −3.337 | 1.00 | 26.88 | 7 | N |
| ATOM | 406 | CA | GLY | A | 71 | 20.513 | −5.309 | −4.312 | 1.00 | 26.41 | 6 | C |
| ATOM | 407 | C | GLY | A | 71 | 19.989 | −4.043 | −3.641 | 1.00 | 25.86 | 6 | C |
| ATOM | 408 | O | GLY | A | 71 | 19.567 | −4.078 | −2.488 | 1.00 | 25.29 | 8 | O |
| ATOM | 409 | N | TYR | A | 72 | 20.013 | −2.924 | −4.350 | 1.00 | 25.62 | 7 | N |
| ATOM | 410 | CA | TYR | A | 72 | 19.566 | −1.666 | −3.745 | 1.00 | 25.82 | 6 | C |
| ATOM | 411 | CB | TYR | A | 72 | 20.731 | −1.032 | −2.949 | 1.00 | 25.61 | 6 | C |
| ATOM | 412 | CG | TYR | A | 72 | 21.915 | −0.698 | −3.831 | 1.00 | 25.97 | 6 | C |
| ATOM | 413 | CD1 | TYR | A | 72 | 21.987 | 0.524 | −4.483 | 1.00 | 26.37 | 6 | C |
| ATOM | 414 | CE1 | TYR | A | 72 | 23.041 | 0.830 | −5.313 | 1.00 | 26.23 | 6 | C |
| ATOM | 415 | CZ | TYR | A | 72 | 24.049 | −0.107 | −5.506 | 1.00 | 26.67 | 6 | C |
| ATOM | 416 | OH | TYR | A | 72 | 25.095 | 0.210 | −6.327 | 1.00 | 27.29 | 8 | O |
| ATOM | 417 | CE2 | TYR | A | 72 | 24.005 | −1.330 | −4.868 | 1.00 | 24.54 | 6 | C |
| ATOM | 418 | CD2 | TYR | A | 72 | 22.941 | −1.626 | −4.044 | 1.00 | 24.92 | 6 | C |
| ATOM | 419 | C | TYR | A | 72 | 19.026 | −0.684 | −4.790 | 1.00 | 25.98 | 6 | C |
| ATOM | 420 | O | TYR | A | 72 | 19.272 | −0.829 | −5.989 | 1.00 | 25.98 | 8 | O |
| ATOM | 421 | N | GLN | A | 73 | 18.243 | 0.287 | −4.325 | 1.00 | 25.67 | 7 | N |
| ATOM | 422 | CA | GLN | A | 73 | 17.746 | 1.349 | −5.172 | 1.00 | 26.04 | 6 | C |
| ATOM | 423 | CB | GLN | A | 73 | 16.218 | 1.439 | −5.100 | 1.00 | 26.48 | 6 | C |
| ATOM | 424 | CG | GLN | A | 73 | 15.642 | 2.564 | −5.972 | 1.00 | 27.53 | 6 | C |
| ATOM | 425 | CD | GLN | A | 73 | 14.208 | 2.947 | −5.601 | 1.00 | 30.17 | 6 | C |
| ATOM | 426 | OE1 | GLN | A | 73 | 13.944 | 3.366 | −4.474 | 1.00 | 28.93 | 8 | O |
| ATOM | 427 | NE2 | GLN | A | 73 | 13.288 | 2.825 | −6.560 | 1.00 | 29.51 | 7 | N |
| ATOM | 428 | C | GLN | A | 73 | 18.329 | 2.675 | −4.660 | 1.00 | 25.84 | 6 | C |
| ATOM | 429 | O | GLN | A | 73 | 18.397 | 2.899 | −3.447 | 1.00 | 25.54 | 8 | O |
| ATOM | 430 | N | LEU | A | 74 | 18.779 | 3.528 | −5.566 | 1.00 | 25.94 | 7 | N |
| ATOM | 431 | CA | LEU | A | 74 | 19.165 | 4.877 | −5.161 | 1.00 | 25.91 | 6 | C |
| ATOM | 432 | CB | LEU | A | 74 | 20.345 | 5.427 | −5.966 | 1.00 | 25.32 | 6 | C |
| ATOM | 433 | CG | LEU | A | 74 | 21.679 | 4.666 | −5.878 | 1.00 | 26.95 | 6 | C |
| ATOM | 434 | CD1 | LEU | A | 74 | 22.775 | 5.388 | −6.660 | 1.00 | 26.40 | 6 | C |
| ATOM | 435 | CD2 | LEU | A | 74 | 22.115 | 4.443 | −4.424 | 1.00 | 27.89 | 6 | C |
| ATOM | 436 | C | LEU | A | 74 | 17.936 | 5.775 | −5.292 | 1.00 | 26.16 | 6 | C |
| ATOM | 437 | O | LEU | A | 74 | 16.957 | 5.420 | −5.958 | 1.00 | 25.70 | 8 | O |
| ATOM | 438 | N | CYS | A | 75 | 17.994 | 6.937 | −4.656 | 1.00 | 25.94 | 7 | N |
| ATOM | 439 | CA | CYS | A | 75 | 16.862 | 7.872 | −4.631 | 1.00 | 27.22 | 6 | C |
| ATOM | 440 | CB | CYS | A | 75 | 17.205 | 9.034 | −3.697 | 1.00 | 26.98 | 6 | C |
| ATOM | 441 | SG | CYS | A | 75 | 18.557 | 10.044 | −4.311 | 1.00 | 28.44 | 16 | S |
| ATOM | 442 | C | CYS | A | 75 | 16.402 | 8.414 | −5.998 | 1.00 | 27.82 | 6 | C |
| ATOM | 443 | O | CYS | A | 75 | 15.287 | 8.945 | −6.120 | 1.00 | 28.35 | 8 | O |
| ATOM | 444 | N | ASP | A | 76 | 17.253 | 8.294 | −7.014 | 1.00 | 28.30 | 7 | N |
| ATOM | 445 | CA | ASP | A | 76 | 16.927 | 8.745 | −8.375 | 1.00 | 29.16 | 6 | C |
| ATOM | 446 | CB | ASP | A | 76 | 18.199 | 9.183 | −9.100 | 1.00 | 29.04 | 6 | C |
| ATOM | 447 | CG | ASP | A | 76 | 19.061 | 7.999 | −9.514 | 1.00 | 30.05 | 6 | C |
| ATOM | 448 | OD1 | ASP | A | 76 | 19.836 | 8.131 | −10.488 | 1.00 | 31.11 | 8 | O |
| ATOM | 449 | OD2 | ASP | A | 76 | 19.018 | 6.897 | −8.925 | 1.00 | 28.83 | 8 | O |
| ATOM | 450 | C | ASP | A | 76 | 16.240 | 7.651 | −9.197 | 1.00 | 29.24 | 6 | C |
| ATOM | 451 | O | ASP | A | 76 | 16.018 | 7.813 | −10.403 | 1.00 | 29.76 | 8 | O |
| ATOM | 452 | N | ASN | A | 77 | 15.929 | 6.541 | −8.534 | 1.00 | 29.36 | 7 | N |
| ATOM | 453 | CA | ASN | A | 77 | 15.269 | 5.367 | −9.117 | 1.00 | 29.49 | 6 | C |
| ATOM | 454 | CB | ASN | A | 77 | 14.035 | 5.757 | −9.938 | 1.00 | 30.31 | 6 | C |
| ATOM | 455 | CG | ASN | A | 77 | 13.039 | 6.559 | −9.121 | 1.00 | 32.03 | 6 | C |
| ATOM | 456 | OD1 | ASN | A | 77 | 12.765 | 6.237 | −7.960 | 1.00 | 32.48 | 8 | O |
| ATOM | 457 | ND2 | ASN | A | 77 | 12.510 | 7.618 | −9.713 | 1.00 | 34.72 | 7 | N |
| ATOM | 458 | C | ASN | A | 77 | 16.169 | 4.355 | −9.860 | 1.00 | 28.92 | 6 | C |
| ATOM | 459 | O | ASN | A | 77 | 15.682 | 3.323 | −10.343 | 1.00 | 28.69 | 8 | O |
| ATOM | 460 | N | SER | A | 78 | 17.465 | 4.652 | −9.943 | 1.00 | 28.22 | 7 | N |
| ATOM | 461 | CA | SER | A | 78 | 18.426 | 3.689 | −10.479 | 1.00 | 27.62 | 6 | C |
| ATOM | 462 | CB | SER | A | 78 | 19.815 | 4.313 | −10.669 | 1.00 | 27.53 | 6 | C |
| ATOM | 463 | OG | SER | A | 78 | 20.396 | 4.720 | −9.427 | 1.00 | 28.06 | 8 | O |
| ATOM | 464 | C | SER | A | 78 | 18.517 | 2.540 | −9.485 | 1.00 | 27.25 | 6 | C |
| ATOM | 465 | O | SER | A | 78 | 18.196 | 2.705 | −8.312 | 1.00 | 26.90 | 8 | O |
| ATOM | 466 | N | VAL | A | 79 | 18.946 | 1.370 | −9.947 | 1.00 | 27.24 | 7 | N |
| ATOM | 467 | CA | VAL | A | 79 | 19.108 | 0.237 | −9.054 | 1.00 | 27.17 | 6 | C |
| ATOM | 468 | CB | VAL | A | 79 | 18.043 | −0.866 | −9.292 | 1.00 | 27.70 | 6 | C |
| ATOM | 469 | CG1 | VAL | A | 79 | 16.627 | −0.363 | −8.933 | 1.00 | 27.98 | 6 | C |
| ATOM | 470 | CG2 | VAL | A | 79 | 18.100 | −1.369 | −10.720 | 1.00 | 28.75 | 6 | C |
| ATOM | 471 | C | VAL | A | 79 | 20.516 | −0.323 | −9.224 | 1.00 | 27.02 | 6 | C |
| ATOM | 472 | O | VAL | A | 79 | 21.173 | −0.094 | −10.238 | 1.00 | 26.92 | 8 | O |
| ATOM | 473 | N | GLY | A | 80 | 20.993 | −1.038 | −8.220 | 1.00 | 26.41 | 7 | N |
| ATOM | 474 | CA | GLY | A | 80 | 22.324 | −1.591 | −8.302 | 1.00 | 26.60 | 6 | C |
| ATOM | 475 | C | GLY | A | 80 | 22.419 | −2.870 | −7.513 | 1.00 | 26.72 | 6 | C |
| ATOM | 476 | O | GLY | A | 80 | 21.537 | −3.203 | −6.730 | 1.00 | 26.68 | 8 | O |
| ATOM | 477 | N | VAL | A | 81 | 23.496 | −3.602 | −7.732 | 1.00 | 27.22 | 7 | N |
| ATOM | 478 | CA | VAL | A | 81 | 23.731 | −4.813 | −6.976 | 1.00 | 27.84 | 6 | C |
| ATOM | 479 | CB | VAL | A | 81 | 23.230 | −6.069 | −7.704 | 1.00 | 27.76 | 6 | C |
| ATOM | 480 | CG1 | VAL | A | 81 | 23.847 | −6.152 | −9.075 | 1.00 | 30.13 | 6 | C |
| ATOM | 481 | CG2 | VAL | A | 81 | 23.561 | −7.335 | −6.893 | 1.00 | 27.79 | 6 | C |
| ATOM | 482 | C | VAL | A | 81 | 25.218 | −4.962 | −6.729 | 1.00 | 27.52 | 6 | C |
| ATOM | 483 | O | VAL | A | 81 | 26.046 | −4.662 | −7.594 | 1.00 | 27.69 | 8 | O |
| ATOM | 484 | N | LEU | A | 82 | 25.551 | −5.387 | −5.524 | 1.00 | 26.88 | 7 | N |
| ATOM | 485 | CA | LEU | A | 82 | 26.921 | −5.726 | −5.211 | 1.00 | 27.04 | 6 | C |
| ATOM | 486 | CB | LEU | A | 82 | 27.370 | −5.067 | −3.907 | 1.00 | 26.63 | 6 | C |
| ATOM | 487 | CG | LEU | A | 82 | 28.677 | −5.575 | −3.286 | 1.00 | 29.59 | 6 | C |
| ATOM | 488 | CD1 | LEU | A | 82 | 29.730 | −5.961 | −4.330 | 1.00 | 31.06 | 6 | C |
| ATOM | 489 | CD2 | LEU | A | 82 | 29.224 | −4.575 | −2.259 | 1.00 | 29.35 | 6 | C |
| ATOM | 490 | C | LEU | A | 82 | 26.851 | −7.239 | −5.115 | 1.00 | 26.34 | 6 | C |
| ATOM | 491 | O | LEU | A | 82 | 26.353 | −7.799 | −4.127 | 1.00 | 26.20 | 8 | O |
| ATOM | 492 | N | PHE | A | 83 | 27.311 | −7.898 | −6.177 | 1.00 | 26.15 | 7 | N |
| ATOM | 493 | CA | PHE | A | 83 | 27.266 | −9.350 | −6.261 | 1.00 | 26.17 | 6 | C |
| ATOM | 494 | CB | PHE | A | 83 | 27.536 | −9.809 | −7.699 | 1.00 | 25.60 | 6 | C |
| ATOM | 495 | CG | PHE | A | 83 | 26.433 | −9.467 | −8.678 | 1.00 | 26.75 | 6 | C |
| ATOM | 496 | CD1 | PHE | A | 83 | 26.644 | −8.533 | −9.676 | 1.00 | 26.59 | 6 | C |
| ATOM | 497 | CE1 | PHE | A | 83 | 25.652 | −8.235 | −10.592 | 1.00 | 27.88 | 6 | C |
| ATOM | 498 | CZ | PHE | A | 83 | 24.432 | −8.871 | −10.510 | 1.00 | 28.25 | 6 | C |
| ATOM | 499 | CE2 | PHE | A | 83 | 24.211 | −9.810 | −9.527 | 1.00 | 28.69 | 6 | C |
| ATOM | 500 | CD2 | PHE | A | 83 | 25.209 | −10.097 | −8.607 | 1.00 | 26.35 | 6 | C |
| ATOM | 501 | C | PHE | A | 83 | 28.275 | −10.036 | −5.338 | 1.00 | 26.24 | 6 | C |
| ATOM | 502 | O | PHE | A | 83 | 29.308 | −9.478 | −4.993 | 1.00 | 25.94 | 8 | O |
| ATOM | 503 | N | ASN | A | 84 | 27.981 | −11.277 | −4.982 | 1.00 | 26.65 | 7 | N |
| ATOM | 504 | CA | ASN | A | 84 | 28.866 | −12.062 | −4.122 | 1.00 | 27.40 | 6 | C |
| ATOM | 505 | CB | ASN | A | 84 | 28.232 | −13.424 | −3.830 | 1.00 | 27.75 | 6 | C |
| ATOM | 506 | CG | ASN | A | 84 | 27.168 | −13.348 | −2.769 | 1.00 | 30.05 | 6 | C |
| ATOM | 507 | OD1 | ASN | A | 84 | 26.839 | −12.261 | −2.277 | 1.00 | 29.88 | 8 | O |
| ATOM | 508 | ND2 | ASN | A | 84 | 26.623 | −14.504 | −2.394 | 1.00 | 29.43 | 7 | N |
| ATOM | 509 | C | ASN | A | 84 | 30.275 | −12.275 | −4.676 | 1.00 | 27.09 | 6 | C |
| ATOM | 510 | O | ASN | A | 84 | 31.189 | −12.621 | −3.929 | 1.00 | 27.36 | 8 | O |
| ATOM | 511 | N | ASN | A | 85 | 30.449 | −12.093 | −5.979 | 1.00 | 26.58 | 7 | N |
| ATOM | 512 | CA | ASN | A | 85 | 31.763 | −12.223 | −6.588 | 1.00 | 26.96 | 6 | C |
| ATOM | 513 | CB | ASN | A | 85 | 31.644 | −12.772 | −8.009 | 1.00 | 27.42 | 6 | C |
| ATOM | 514 | CG | ASN | A | 85 | 30.946 | −11.795 | −8.950 | 1.00 | 26.92 | 6 | C |
| ATOM | 515 | OD1 | ASN | A | 85 | 30.513 | −10.720 | −8.539 | 1.00 | 29.27 | 8 | O |
| ATOM | 516 | ND2 | ASN | A | 85 | 30.851 | −12.158 | −10.211 | 1.00 | 26.62 | 7 | N |
| ATOM | 517 | C | ASN | A | 85 | 32.534 | −10.898 | −6.618 | 1.00 | 26.95 | 6 | C |
| ATOM | 518 | O | ASN | A | 85 | 33.563 | −10.794 | −7.273 | 1.00 | 26.21 | 8 | O |
| ATOM | 519 | N | SER | A | 86 | 32.010 | −9.893 | −5.919 | 1.00 | 27.52 | 7 | N |
| ATOM | 520 | CA | SER | A | 86 | 32.627 | −8.563 | −5.827 | 1.00 | 27.99 | 6 | C |
| ATOM | 521 | CB | SER | A | 86 | 34.076 | −8.638 | −5.354 | 1.00 | 28.25 | 6 | C |
| ATOM | 522 | OG | SER | A | 86 | 34.114 | −9.058 | −4.012 | 1.00 | 31.12 | 8 | O |
| ATOM | 523 | C | SER | A | 86 | 32.544 | −7.699 | −7.079 | 1.00 | 27.67 | 6 | C |
| ATOM | 524 | O | SER | A | 86 | 33.267 | −6.704 | −7.197 | 1.00 | 28.16 | 8 | O |
| ATOM | 525 | N | THR | A | 87 | 31.690 | −8.067 | −8.018 | 1.00 | 26.97 | 7 | N |
| ATOM | 526 | CA | THR | A | 87 | 31.472 | −7.178 | −9.157 | 1.00 | 26.07 | 6 | C |
| ATOM | 527 | CB | THR | A | 87 | 31.330 | −7.954 | −10.466 | 1.00 | 25.82 | 6 | C |
| ATOM | 528 | OG1 | THR | A | 87 | 30.155 | −8.782 | −10.416 | 1.00 | 23.37 | 8 | O |
| ATOM | 529 | CG2 | THR | A | 87 | 32.496 | −8.954 | −10.633 | 1.00 | 24.35 | 6 | C |
| ATOM | 530 | C | THR | A | 87 | 30.203 | −6.416 | −8.844 | 1.00 | 26.76 | 6 | C |
| ATOM | 531 | O | THR | A | 87 | 29.409 | −6.851 | −8.002 | 1.00 | 26.49 | 8 | O |
| ATOM | 532 | N | ARG | A | 88 | 30.007 | −5.282 | −9.504 | 1.00 | 26.58 | 7 | N |
| ATOM | 533 | CA | ARG | A | 88 | 28.807 | −4.486 | −9.277 | 1.00 | 27.64 | 6 | C |
| ATOM | 534 | CB | ARG | A | 88 | 29.136 | −3.257 | −8.418 | 1.00 | 28.83 | 6 | C |
| ATOM | 535 | CG | ARG | A | 88 | 30.493 | −3.375 | −7.687 | 1.00 | 32.71 | 6 | C |
| ATOM | 536 | CD | ARG | A | 88 | 30.554 | −2.751 | −6.330 | 1.00 | 38.97 | 6 | C |
| ATOM | 537 | NE | ARG | A | 88 | 31.926 | −2.457 | −5.938 | 1.00 | 42.42 | 7 | N |
| ATOM | 538 | CZ | ARG | A | 88 | 32.537 | −2.966 | −4.878 | 1.00 | 44.46 | 6 | C |
| ATOM | 539 | NH1 | ARG | A | 88 | 31.909 | −3.806 | −4.081 | 1.00 | 47.01 | 7 | N |
| ATOM | 540 | NH2 | ARG | A | 88 | 33.789 | −2.628 | −4.607 | 1.00 | 46.70 | 7 | N |
| ATOM | 541 | C | ARG | A | 88 | 28.184 | −4.088 | −10.610 | 1.00 | 27.27 | 6 | C |
| ATOM | 542 | O | ARG | A | 88 | 28.891 | −3.834 | −11.582 | 1.00 | 26.57 | 8 | O |
| ATOM | 543 | N | LEU | A | 89 | 26.859 | −4.039 | −10.647 | 1.00 | 27.53 | 7 | N |
| ATOM | 544 | CA | LEU | A | 89 | 26.136 | −3.680 | −11.859 | 1.00 | 27.62 | 6 | C |
| ATOM | 545 | CB | LEU | A | 89 | 25.461 | −4.914 | −12.447 | 1.00 | 27.92 | 6 | C |
| ATOM | 546 | CG | LEU | A | 89 | 24.688 | −4.784 | −13.759 | 1.00 | 28.76 | 6 | C |
| ATOM | 547 | CD1 | LEU | A | 89 | 25.579 | −4.250 | −14.882 | 1.00 | 29.33 | 6 | C |
| ATOM | 548 | CD2 | LEU | A | 89 | 24.090 | −6.152 | −14.140 | 1.00 | 30.55 | 6 | C |
| ATOM | 549 | C | LEU | A | 89 | 25.083 | −2.647 | −11.492 | 1.00 | 27.77 | 6 | C |
| ATOM | 550 | O | LEU | A | 89 | 24.386 | −2.790 | −10.482 | 1.00 | 27.57 | 8 | O |
| ATOM | 551 | N | ILE | A | 90 | 24.982 | −1.604 | −12.308 | 1.00 | 27.83 | 7 | N |
| ATOM | 552 | CA | ILE | A | 90 | 24.023 | −0.532 | −12.076 | 1.00 | 28.43 | 6 | C |
| ATOM | 553 | CB | ILE | A | 90 | 24.763 | 0.807 | −11.845 | 1.00 | 28.80 | 6 | C |
| ATOM | 554 | CG1 | ILE | A | 90 | 25.556 | 0.776 | −10.538 | 1.00 | 30.29 | 6 | C |
| ATOM | 555 | CD1 | ILE | A | 90 | 26.784 | −0.098 | −10.613 | 1.00 | 35.17 | 6 | C |
| ATOM | 556 | CG2 | ILE | A | 90 | 23.783 | 1.978 | −11.861 | 1.00 | 29.24 | 6 | C |
| ATOM | 557 | C | ILE | A | 90 | 23.133 | −0.383 | −13.296 | 1.00 | 28.55 | 6 | C |
| ATOM | 558 | O | ILE | A | 90 | 23.617 | −0.415 | −14.422 | 1.00 | 28.28 | 8 | O |
| ATOM | 559 | N | LEU | A | 91 | 21.836 | −0.227 | −13.065 | 1.00 | 28.68 | 7 | N |
| ATOM | 560 | CA | LEU | A | 91 | 20.892 | 0.034 | −14.139 | 1.00 | 29.39 | 6 | C |
| ATOM | 561 | CB | LEU | A | 91 | 19.790 | −1.021 | −14.138 | 1.00 | 29.32 | 6 | C |
| ATOM | 562 | CG | LEU | A | 91 | 18.627 | −0.740 | −15.096 | 1.00 | 30.30 | 6 | C |
| ATOM | 563 | CD1 | LEU | A | 91 | 19.064 | −0.984 | −16.540 | 1.00 | 30.54 | 6 | C |
| ATOM | 564 | CD2 | LEU | A | 91 | 17.408 | −1.595 | −14.738 | 1.00 | 30.62 | 6 | C |
| ATOM | 565 | C | LEU | A | 91 | 20.329 | 1.441 | −13.885 | 1.00 | 29.83 | 6 | C |
| ATOM | 566 | O | LEU | A | 91 | 19.727 | 1.695 | −12.830 | 1.00 | 29.50 | 8 | O |
| ATOM | 567 | N | TYR | A | 92 | 20.579 | 2.358 | −14.821 | 1.00 | 30.23 | 7 | N |
| ATOM | 568 | CA | TYR | A | 92 | 20.127 | 3.751 | −14.697 | 1.00 | 30.91 | 6 | C |
| ATOM | 569 | CB | TYR | A | 92 | 20.768 | 4.626 | −15.772 | 1.00 | 30.86 | 6 | C |
| ATOM | 570 | CG | TYR | A | 92 | 22.249 | 4.831 | −15.576 | 1.00 | 32.76 | 6 | C |
| ATOM | 571 | CD1 | TYR | A | 92 | 23.140 | 3.777 | −15.715 | 1.00 | 33.72 | 6 | C |
| ATOM | 572 | CE1 | TYR | A | 92 | 24.493 | 3.958 | −15.528 | 1.00 | 35.16 | 6 | C |
| ATOM | 573 | CZ | TYR | A | 92 | 24.973 | 5.204 | −15.199 | 1.00 | 34.76 | 6 | C |
| ATOM | 574 | OH | TYR | A | 92 | 26.318 | 5.388 | −15.021 | 1.00 | 35.17 | 8 | O |
| ATOM | 575 | CE2 | TYR | A | 92 | 24.115 | 6.268 | −15.056 | 1.00 | 34.84 | 6 | C |
| ATOM | 576 | CD2 | TYR | A | 92 | 22.758 | 6.078 | −15.238 | 1.00 | 34.70 | 6 | C |
| ATOM | 577 | C | TYR | A | 92 | 18.610 | 3.886 | −14.743 | 1.00 | 31.31 | 6 | C |
| ATOM | 578 | O | TYR | A | 92 | 17.918 | 2.977 | −15.190 | 1.00 | 31.18 | 8 | O |
| ATOM | 579 | N | ASN | A | 93 | 18.096 | 5.028 | −14.283 | 1.00 | 32.12 | 7 | N |
| ATOM | 580 | CA | ASN | A | 93 | 16.650 | 5.235 | −14.234 | 1.00 | 33.32 | 6 | C |
| ATOM | 581 | CB | ASN | A | 93 | 16.261 | 6.432 | −13.344 | 1.00 | 33.25 | 6 | C |
| ATOM | 582 | CG | ASN | A | 93 | 16.786 | 7.768 | −13.866 | 1.00 | 33.58 | 6 | C |
| ATOM | 583 | OD1 | ASN | A | 93 | 17.268 | 7.874 | −14.998 | 1.00 | 32.74 | 8 | O |
| ATOM | 584 | ND2 | ASN | A | 93 | 16.697 | 8.802 | −13.025 | 1.00 | 33.00 | 7 | N |
| ATOM | 585 | C | ASN | A | 93 | 15.951 | 5.319 | −15.598 | 1.00 | 34.12 | 6 | C |
| ATOM | 586 | O | ASN | A | 93 | 14.728 | 5.469 | −15.661 | 1.00 | 34.09 | 8 | O |
| ATOM | 587 | N | ASP | A | 94 | 16.708 | 5.244 | −16.687 | 1.00 | 34.86 | 7 | N |
| ATOM | 588 | CA | ASP | A | 94 | 16.053 | 5.211 | −17.998 | 1.00 | 35.81 | 6 | C |
| ATOM | 589 | CB | ASP | A | 94 | 16.846 | 5.942 | −19.087 | 1.00 | 36.25 | 6 | C |
| ATOM | 590 | CG | ASP | A | 94 | 18.336 | 5.762 | −18.951 | 1.00 | 38.64 | 6 | C |
| ATOM | 591 | OD1 | ASP | A | 94 | 18.966 | 5.230 | −19.897 | 1.00 | 36.80 | 8 | O |
| ATOM | 592 | OD2 | ASP | A | 94 | 18.964 | 6.124 | −17.927 | 1.00 | 44.80 | 8 | O |
| ATOM | 593 | C | ASP | A | 94 | 15.721 | 3.777 | −18.384 | 1.00 | 35.67 | 6 | C |
| ATOM | 594 | O | ASP | A | 94 | 15.157 | 3.522 | −19.453 | 1.00 | 35.81 | 8 | O |
| ATOM | 595 | N | GLY | A | 95 | 16.071 | 2.846 | −17.498 | 1.00 | 35.30 | 7 | N |
| ATOM | 596 | CA | GLY | A | 95 | 15.730 | 1.441 | −17.658 | 1.00 | 34.65 | 6 | C |
| ATOM | 597 | C | GLY | A | 95 | 16.512 | 0.649 | −18.693 | 1.00 | 34.46 | 6 | C |
| ATOM | 598 | O | GLY | A | 95 | 16.176 | −0.506 | −18.967 | 1.00 | 34.40 | 8 | O |
| ATOM | 599 | N | ASP | A | 96 | 17.563 | 1.242 | −19.250 | 1.00 | 34.02 | 7 | N |
| ATOM | 600 | CA | ASP | A | 96 | 18.339 | 0.568 | −20.293 | 1.00 | 33.87 | 6 | C |
| ATOM | 601 | CB | ASP | A | 96 | 17.887 | 1.059 | −21.675 | 1.00 | 33.75 | 6 | C |
| ATOM | 602 | CG | ASP | A | 96 | 18.427 | 0.203 | −22.811 | 1.00 | 34.99 | 6 | C |
| ATOM | 603 | OD1 | ASP | A | 96 | 18.663 | −1.003 | −22.608 | 1.00 | 34.90 | 8 | O |
| ATOM | 604 | OD2 | ASP | A | 96 | 18.647 | 0.659 | −23.949 | 1.00 | 36.72 | 8 | O |
| ATOM | 605 | C | ASP | A | 96 | 19.852 | 0.741 | −20.129 | 1.00 | 33.04 | 6 | C |
| ATOM | 606 | O | ASP | A | 96 | 20.624 | −0.175 | −20.402 | 1.00 | 33.28 | 8 | O |
| ATOM | 607 | N | SER | A | 97 | 20.280 | 1.911 | −19.671 | 1.00 | 32.38 | 7 | N |
| ATOM | 608 | CA | SER | A | 97 | 21.709 | 2.180 | −19.519 | 1.00 | 31.14 | 6 | C |
| ATOM | 609 | CB | SER | A | 97 | 21.951 | 3.681 | −19.327 | 1.00 | 31.84 | 6 | C |
| ATOM | 610 | OG | SER | A | 97 | 21.665 | 4.395 | −20.523 | 1.00 | 31.58 | 8 | O |
| ATOM | 611 | C | SER | A | 97 | 22.330 | 1.387 | −18.364 | 1.00 | 30.82 | 6 | C |
| ATOM | 612 | O | SER | A | 97 | 21.708 | 1.223 | −17.307 | 1.00 | 29.82 | 8 | O |
| ATOM | 613 | N | LEU | A | 98 | 23.545 | 0.892 | −18.575 | 1.00 | 29.55 | 7 | N |
| ATOM | 614 | CA | LEU | A | 98 | 24.223 | 0.093 | −17.559 | 1.00 | 29.94 | 6 | C |
| ATOM | 615 | CB | LEU | A | 98 | 24.370 | −1.359 | −18.022 | 1.00 | 29.99 | 6 | C |
| ATOM | 616 | CG | LEU | A | 98 | 23.131 | −2.222 | −18.248 | 1.00 | 29.63 | 6 | C |
| ATOM | 617 | CD1 | LEU | A | 98 | 23.554 | −3.504 | −18.966 | 1.00 | 30.60 | 6 | C |
| ATOM | 618 | CD2 | LEU | A | 98 | 22.433 | −2.544 | −16.933 | 1.00 | 29.49 | 6 | C |
| ATOM | 619 | C | LEU | A | 98 | 25.612 | 0.614 | −17.275 | 1.00 | 29.90 | 6 | C |
| ATOM | 620 | O | LEU | A | 98 | 26.267 | 1.186 | −18.152 | 1.00 | 29.73 | 8 | O |
| ATOM | 621 | N | GLN | A | 99 | 26.055 | 0.397 | −16.040 | 1.00 | 29.63 | 7 | N |
| ATOM | 622 | CA | GLN | A | 99 | 27.436 | 0.632 | −15.656 | 1.00 | 29.72 | 6 | C |
| ATOM | 623 | CB | GLN | A | 99 | 27.580 | 1.834 | −14.710 | 1.00 | 29.87 | 6 | C |
| ATOM | 624 | CG | GLN | A | 99 | 29.006 | 2.022 | −14.179 | 1.00 | 31.65 | 6 | C |
| ATOM | 625 | CD | GLN | A | 99 | 29.154 | 3.179 | −13.176 | 1.00 | 34.86 | 6 | C |
| ATOM | 626 | OE1 | GLN | A | 99 | 28.225 | 3.975 | −12.969 | 1.00 | 38.05 | 8 | O |
| ATOM | 627 | NE2 | GLN | A | 99 | 30.320 | 3.268 | −12.558 | 1.00 | 35.00 | 7 | N |
| ATOM | 628 | C | GLN | A | 99 | 27.878 | −0.649 | −14.948 | 1.00 | 29.09 | 6 | C |
| ATOM | 629 | O | GLN | A | 99 | 27.285 | −1.027 | −13.939 | 1.00 | 28.62 | 8 | O |
| ATOM | 630 | N | TYR | A | 100 | 28.885 | −1.326 | −15.496 | 1.00 | 28.74 | 7 | N |
| ATOM | 631 | CA | TYR | A | 100 | 29.428 | −2.553 | −14.902 | 1.00 | 28.36 | 6 | C |
| ATOM | 632 | CB | TYR | A | 100 | 29.588 | −3.661 | −15.952 | 1.00 | 28.26 | 6 | C |
| ATOM | 633 | CG | TYR | A | 100 | 29.947 | −5.033 | −15.395 | 1.00 | 28.17 | 6 | C |
| ATOM | 634 | CD1 | TYR | A | 100 | 29.237 | −5.587 | −14.329 | 1.00 | 28.77 | 6 | C |
| ATOM | 635 | CE1 | TYR | A | 100 | 29.544 | −6.851 | −13.835 | 1.00 | 28.30 | 6 | C |
| ATOM | 636 | CZ | TYR | A | 100 | 30.580 | −7.566 | −14.398 | 1.00 | 27.40 | 6 | C |
| ATOM | 637 | OH | TYR | A | 100 | 30.886 | −8.818 | −13.917 | 1.00 | 27.32 | 8 | O |
| ATOM | 638 | CE2 | TYR | A | 100 | 31.303 | −7.029 | −15.446 | 1.00 | 27.53 | 6 | C |
| ATOM | 639 | CD2 | TYR | A | 100 | 30.981 | −5.782 | −15.941 | 1.00 | 27.24 | 6 | C |
| ATOM | 640 | C | TYR | A | 100 | 30.782 | −2.244 | −14.300 | 1.00 | 28.71 | 6 | C |
| ATOM | 641 | O | TYR | A | 100 | 31.638 | −1.639 | −14.963 | 1.00 | 29.05 | 8 | O |
| ATOM | 642 | N | ILE | A | 101 | 30.973 | −2.630 | −13.040 | 1.00 | 28.17 | 7 | N |
| ATOM | 643 | CA | ILE | A | 101 | 32.246 | −2.421 | −12.363 | 1.00 | 28.15 | 6 | C |
| ATOM | 644 | CB | ILE | A | 101 | 32.094 | −1.589 | −11.065 | 1.00 | 28.44 | 6 | C |
| ATOM | 645 | CG1 | ILE | A | 101 | 31.429 | −0.235 | −11.339 | 1.00 | 29.47 | 6 | C |
| ATOM | 646 | CD1 | ILE | A | 101 | 29.939 | −0.276 | −11.252 | 1.00 | 32.69 | 6 | C |
| ATOM | 647 | CG2 | ILE | A | 101 | 33.444 | −1.322 | −10.461 | 1.00 | 28.54 | 6 | C |
| ATOM | 648 | C | ILE | A | 101 | 32.816 | −3.778 | −12.014 | 1.00 | 28.30 | 6 | C |
| ATOM | 649 | O | ILE | A | 101 | 32.228 | −4.518 | −11.212 | 1.00 | 26.96 | 8 | O |
| ATOM | 650 | N | GLU | A | 102 | 33.951 | −4.099 | −12.625 | 1.00 | 28.36 | 7 | N |
| ATOM | 651 | CA | GLU | A | 102 | 34.618 | −5.373 | −12.391 | 1.00 | 29.48 | 6 | C |
| ATOM | 652 | CB | GLU | A | 102 | 35.545 | −5.713 | −13.558 | 1.00 | 29.40 | 6 | C |
| ATOM | 653 | CG | GLU | A | 102 | 34.783 | −6.010 | −14.839 | 1.00 | 29.63 | 6 | C |
| ATOM | 654 | CD | GLU | A | 102 | 35.686 | −6.094 | −16.049 | 1.00 | 31.22 | 6 | C |
| ATOM | 655 | OE1 | GLU | A | 102 | 36.394 | −7.111 | −16.206 | 1.00 | 31.47 | 8 | O |
| ATOM | 656 | OE2 | GLU | A | 102 | 35.691 | −5.132 | −16.841 | 1.00 | 34.12 | 8 | O |
| ATOM | 657 | C | GLU | A | 102 | 35.380 | −5.349 | −11.072 | 1.00 | 30.52 | 6 | C |
| ATOM | 658 | O | GLU | A | 102 | 35.519 | −4.299 | −10.446 | 1.00 | 29.92 | 8 | O |
| ATOM | 659 | N | ARG | A | 103 | 35.854 | −6.518 | −10.647 | 1.00 | 31.46 | 7 | N |
| ATOM | 660 | CA | ARG | A | 103 | 36.540 | −6.644 | −9.365 | 1.00 | 33.21 | 6 | C |
| ATOM | 661 | CB | ARG | A | 103 | 37.104 | −8.056 | −9.195 | 1.00 | 33.45 | 6 | C |
| ATOM | 662 | CG | ARG | A | 103 | 36.036 | −9.120 | −9.017 | 1.00 | 35.12 | 6 | C |
| ATOM | 663 | CD | ARG | A | 103 | 36.541 | −10.534 | −9.294 | 1.00 | 39.05 | 6 | C |
| ATOM | 664 | NE | ARG | A | 103 | 37.045 | −11.209 | −8.112 | 1.00 | 42.34 | 7 | N |
| ATOM | 665 | CZ | ARG | A | 103 | 38.106 | −12.010 | −8.100 | 1.00 | 43.44 | 6 | C |
| ATOM | 666 | NH1 | ARG | A | 103 | 38.817 | −12.210 | −9.204 | 1.00 | 44.15 | 7 | N |
| ATOM | 667 | NH2 | ARG | A | 103 | 38.474 | −12.594 | −6.972 | 1.00 | 44.98 | 7 | N |
| ATOM | 668 | C | ARG | A | 103 | 37.659 | −5.641 | −9.179 | 1.00 | 33.82 | 6 | C |
| ATOM | 669 | O | ARG | A | 103 | 37.835 | −5.100 | −8.101 | 1.00 | 34.04 | 8 | O |
| ATOM | 670 | N | ASP | A | 104 | 38.415 | −5.400 | −10.236 | 1.00 | 35.17 | 7 | N |
| ATOM | 671 | CA | ASP | A | 104 | 39.541 | −4.483 | −10.173 | 1.00 | 36.39 | 6 | C |
| ATOM | 672 | CB | ASP | A | 104 | 40.521 | −4.855 | −11.266 | 1.00 | 37.19 | 6 | C |
| ATOM | 673 | CG | ASP | A | 104 | 39.825 | −5.106 | −12.580 | 1.00 | 40.35 | 6 | C |
| ATOM | 674 | OD1 | ASP | A | 104 | 39.635 | −6.296 | −12.946 | 1.00 | 45.85 | 8 | O |
| ATOM | 675 | OD2 | ASP | A | 104 | 39.375 | −4.177 | −13.279 | 1.00 | 39.52 | 8 | O |
| ATOM | 676 | C | ASP | A | 104 | 39.114 | −3.023 | −10.344 | 1.00 | 36.08 | 6 | C |
| ATOM | 677 | O | ASP | A | 104 | 39.958 | −2.139 | −10.469 | 1.00 | 36.56 | 8 | O |
| ATOM | 678 | N | GLY | A | 105 | 37.812 | −2.768 | −10.359 | 1.00 | 35.42 | 7 | N |
| ATOM | 679 | CA | GLY | A | 105 | 37.328 | −1.407 | −10.502 | 1.00 | 35.09 | 6 | C |
| ATOM | 680 | C | GLY | A | 105 | 37.112 | −0.922 | −11.930 | 1.00 | 34.77 | 6 | C |
| ATOM | 681 | O | GLY | A | 105 | 36.659 | 0.201 | −12.136 | 1.00 | 34.65 | 8 | O |
| ATOM | 682 | N | THR | A | 106 | 37.413 | −1.754 | −12.922 | 1.00 | 34.37 | 7 | N |
| ATOM | 683 | CA | THR | A | 106 | 37.198 | −1.344 | −14.315 | 1.00 | 34.21 | 6 | C |
| ATOM | 684 | CB | THR | A | 106 | 37.736 | −2.400 | −15.295 | 1.00 | 34.25 | 6 | C |
| ATOM | 685 | OG1 | THR | A | 106 | 39.147 | −2.568 | −15.095 | 1.00 | 33.88 | 8 | O |
| ATOM | 686 | CG2 | THR | A | 106 | 37.638 | −1.888 | −16.738 | 1.00 | 34.18 | 6 | C |
| ATOM | 687 | C | THR | A | 106 | 35.713 | −1.081 | −14.577 | 1.00 | 34.43 | 6 | C |
| ATOM | 688 | O | THR | A | 106 | 34.864 | −1.919 | −14.263 | 1.00 | 33.73 | 8 | O |
| ATOM | 689 | N | GLU | A | 107 | 35.401 | 0.080 | −15.154 | 1.00 | 34.65 | 7 | N |
| ATOM | 690 | CA | GLU | A | 107 | 34.009 | 0.456 | −15.410 | 1.00 | 35.83 | 6 | C |
| ATOM | 691 | CB | GLU | A | 107 | 33.721 | 1.882 | −14.917 | 1.00 | 35.89 | 6 | C |
| ATOM | 692 | CG | GLU | A | 107 | 33.975 | 2.149 | −13.440 | 1.00 | 38.03 | 6 | C |
| ATOM | 693 | CD | GLU | A | 107 | 33.784 | 3.616 | −13.066 | 1.00 | 40.69 | 6 | C |
| ATOM | 694 | OE1 | GLU | A | 107 | 33.359 | 4.413 | −13.938 | 1.00 | 42.02 | 8 | O |
| ATOM | 695 | OE2 | GLU | A | 107 | 34.062 | 3.982 | −11.902 | 1.00 | 42.05 | 8 | O |
| ATOM | 696 | C | GLU | A | 107 | 33.649 | 0.369 | −16.890 | 1.00 | 35.91 | 6 | C |
| ATOM | 697 | O | GLU | A | 107 | 34.394 | 0.848 | −17.750 | 1.00 | 36.37 | 8 | O |
| ATOM | 698 | N | SER | A | 108 | 32.508 | −0.244 | −17.182 | 1.00 | 35.87 | 7 | N |
| ATOM | 699 | CA | SER | A | 108 | 32.015 | −0.356 | −18.551 | 1.00 | 36.36 | 6 | C |
| ATOM | 700 | CB | SER | A | 108 | 31.931 | −1.820 | −18.984 | 1.00 | 36.01 | 6 | C |
| ATOM | 701 | OG | SER | A | 108 | 33.217 | −2.393 | −19.154 | 1.00 | 36.34 | 8 | O |
| ATOM | 702 | C | SER | A | 108 | 30.624 | 0.261 | −18.612 | 1.00 | 36.72 | 6 | C |
| ATOM | 703 | O | SER | A | 108 | 29.822 | 0.043 | −17.708 | 1.00 | 36.15 | 8 | O |
| ATOM | 704 | N | TYR | A | 109 | 30.353 | 1.044 | −19.658 | 1.00 | 36.80 | 7 | N |
| ATOM | 705 | CA | TYR | A | 109 | 29.036 | 1.654 | −19.838 | 1.00 | 37.56 | 6 | C |
| ATOM | 706 | CB | TYR | A | 109 | 29.135 | 3.179 | −19.970 | 1.00 | 37.52 | 6 | C |
| ATOM | 707 | CG | TYR | A | 109 | 29.685 | 3.801 | −18.705 | 1.00 | 38.15 | 6 | C |
| ATOM | 708 | CD1 | TYR | A | 109 | 31.044 | 3.771 | −18.435 | 1.00 | 39.00 | 6 | C |
| ATOM | 709 | CE1 | TYR | A | 109 | 31.560 | 4.308 | −17.266 | 1.00 | 38.75 | 6 | C |
| ATOM | 710 | CZ | TYR | A | 109 | 30.710 | 4.878 | −16.347 | 1.00 | 39.50 | 6 | C |
| ATOM | 711 | OH | TYR | A | 109 | 31.236 | 5.409 | −15.186 | 1.00 | 39.40 | 8 | O |
| ATOM | 712 | CE2 | TYR | A | 109 | 29.346 | 4.909 | −16.586 | 1.00 | 39.18 | 6 | C |
| ATOM | 713 | CD2 | TYR | A | 109 | 28.842 | 4.366 | −17.759 | 1.00 | 38.80 | 6 | C |
| ATOM | 714 | C | TYR | A | 109 | 28.365 | 1.011 | −21.036 | 1.00 | 37.99 | 6 | C |
| ATOM | 715 | O | TYR | A | 109 | 28.862 | 1.095 | −22.159 | 1.00 | 38.19 | 8 | O |
| ATOM | 716 | N | LEU | A | 110 | 27.251 | 0.338 | −20.777 | 1.00 | 38.47 | 7 | N |
| ATOM | 717 | CA | LEU | A | 110 | 26.557 | −0.425 | −21.803 | 1.00 | 39.08 | 6 | C |
| ATOM | 718 | CB | LEU | A | 110 | 26.799 | −1.926 | −21.594 | 1.00 | 39.57 | 6 | C |
| ATOM | 719 | CG | LEU | A | 110 | 28.213 | −2.444 | −21.365 | 1.00 | 40.82 | 6 | C |
| ATOM | 720 | CD1 | LEU | A | 110 | 28.195 | −3.950 | −21.138 | 1.00 | 41.92 | 6 | C |
| ATOM | 721 | CD2 | LEU | A | 110 | 29.094 | −2.098 | −22.558 | 1.00 | 42.45 | 6 | C |
| ATOM | 722 | C | LEU | A | 110 | 25.067 | −0.202 | −21.738 | 1.00 | 38.83 | 6 | C |
| ATOM | 723 | O | LEU | A | 110 | 24.565 | 0.584 | −20.932 | 1.00 | 38.56 | 8 | O |
| ATOM | 724 | N | THR | A | 111 | 24.358 | −0.912 | −22.605 | 1.00 | 38.56 | 7 | N |
| ATOM | 725 | CA | THR | A | 111 | 22.911 | −0.869 | −22.607 | 1.00 | 38.47 | 6 | C |
| ATOM | 726 | CB | THR | A | 111 | 22.403 | −0.170 | −23.884 | 1.00 | 38.99 | 6 | C |
| ATOM | 727 | OG1 | THR | A | 111 | 22.813 | 1.205 | −23.879 | 1.00 | 40.54 | 8 | O |
| ATOM | 728 | CG2 | THR | A | 111 | 20.922 | −0.042 | −23.840 | 1.00 | 40.63 | 6 | C |
| ATOM | 729 | C | THR | A | 111 | 22.389 | −2.300 | −22.523 | 1.00 | 37.37 | 6 | C |
| ATOM | 730 | O | THR | A | 111 | 23.043 | −3.234 | −23.004 | 1.00 | 37.13 | 8 | O |
| ATOM | 731 | N | VAL | A | 112 | 21.225 | −2.480 | −21.908 | 1.00 | 36.49 | 7 | N |
| ATOM | 732 | CA | VAL | A | 112 | 20.606 | −3.797 | −21.842 | 1.00 | 36.02 | 6 | C |
| ATOM | 733 | CB | VAL | A | 112 | 19.387 | −3.814 | −20.899 | 1.00 | 36.12 | 6 | C |
| ATOM | 734 | CG1 | VAL | A | 112 | 18.649 | −5.153 | −20.973 | 1.00 | 35.72 | 6 | C |
| ATOM | 735 | CG2 | VAL | A | 112 | 19.819 | −3.524 | −19.464 | 1.00 | 35.10 | 6 | C |
| ATOM | 736 | C | VAL | A | 112 | 20.179 | −4.189 | −23.260 | 1.00 | 36.45 | 6 | C |
| ATOM | 737 | O | VAL | A | 112 | 20.398 | −5.317 | −23.696 | 1.00 | 35.73 | 8 | O |
| ATOM | 738 | N | SER | A | 113 | 19.607 | −3.229 | −23.980 | 1.00 | 36.64 | 7 | N |
| ATOM | 739 | CA | SER | A | 113 | 19.122 | −3.462 | −25.338 | 1.00 | 37.54 | 6 | C |
| ATOM | 740 | CB | SER | A | 113 | 18.484 | −2.190 | −25.912 | 1.00 | 37.50 | 6 | C |
| ATOM | 741 | OG | SER | A | 113 | 19.411 | −1.120 | −25.988 | 1.00 | 38.07 | 8 | O |
| ATOM | 742 | C | SER | A | 113 | 20.203 | −3.999 | −26.273 | 1.00 | 37.96 | 6 | C |
| ATOM | 743 | O | SER | A | 113 | 19.897 | −4.727 | −27.224 | 1.00 | 38.50 | 8 | O |
| ATOM | 744 | N | SER | A | 114 | 21.459 | −3.648 | −26.005 | 1.00 | 38.01 | 7 | N |
| ATOM | 745 | CA | SER | A | 114 | 22.583 | −4.107 | −26.826 | 1.00 | 38.47 | 6 | C |
| ATOM | 746 | CB | SER | A | 114 | 23.839 | −3.268 | −26.562 | 1.00 | 38.34 | 6 | C |
| ATOM | 747 | OG | SER | A | 114 | 24.459 | −3.627 | −25.338 | 1.00 | 38.19 | 8 | O |
| ATOM | 748 | C | SER | A | 114 | 22.910 | −5.588 | −26.628 | 1.00 | 38.68 | 6 | C |
| ATOM | 749 | O | SER | A | 114 | 23.719 | −6.155 | −27.370 | 1.00 | 38.77 | 8 | O |
| ATOM | 750 | N | HIS | A | 115 | 22.296 | −6.199 | −25.619 | 1.00 | 38.57 | 7 | N |
| ATOM | 751 | CA | HIS | A | 115 | 22.502 | −7.616 | −25.308 | 1.00 | 38.72 | 6 | C |
| ATOM | 752 | CB | HIS | A | 115 | 21.892 | −8.516 | −26.398 | 1.00 | 39.09 | 6 | C |
| ATOM | 753 | CG | HIS | A | 115 | 21.849 | −9.966 | −26.026 | 1.00 | 39.56 | 6 | C |
| ATOM | 754 | ND1 | HIS | A | 115 | 20.675 | −10.623 | −25.724 | 1.00 | 40.68 | 7 | N |
| ATOM | 755 | CE1 | HIS | A | 115 | 20.941 | −11.882 | −25.428 | 1.00 | 40.16 | 6 | C |
| ATOM | 756 | NE2 | HIS | A | 115 | 22.246 | −12.066 | −25.525 | 1.00 | 40.59 | 7 | N |
| ATOM | 757 | CD2 | HIS | A | 115 | 22.836 | −10.884 | −25.899 | 1.00 | 39.60 | 6 | C |
| ATOM | 758 | C | HIS | A | 115 | 23.969 | −7.997 | −25.067 | 1.00 | 38.66 | 6 | C |
| ATOM | 759 | O | HIS | A | 115 | 24.575 | −8.716 | −25.871 | 1.00 | 38.76 | 8 | O |
| ATOM | 760 | N | PRO | A | 116 | 24.538 | −7.509 | −23.969 | 1.00 | 38.46 | 7 | N |
| ATOM | 761 | CA | PRO | A | 116 | 25.911 | −7.855 | −23.578 | 1.00 | 38.23 | 6 | C |
| ATOM | 762 | CB | PRO | A | 116 | 26.177 | −6.905 | −22.412 | 1.00 | 38.56 | 6 | C |
| ATOM | 763 | CG | PRO | A | 116 | 24.818 | −6.660 | −21.847 | 1.00 | 38.47 | 6 | C |
| ATOM | 764 | CD | PRO | A | 116 | 23.924 | −6.544 | −23.039 | 1.00 | 38.20 | 6 | C |
| ATOM | 765 | C | PRO | A | 116 | 25.963 | −9.307 | −23.103 | 1.00 | 37.95 | 6 | C |
| ATOM | 766 | O | PRO | A | 116 | 25.567 | −9.620 | −21.977 | 1.00 | 37.64 | 8 | O |
| ATOM | 767 | N | ASN | A | 117 | 26.460 | −10.188 | −23.966 | 1.00 | 37.62 | 7 | N |
| ATOM | 768 | CA | ASN | A | 117 | 26.448 | −11.631 | −23.712 | 1.00 | 37.09 | 6 | C |
| ATOM | 769 | CB | ASN | A | 117 | 27.238 | −12.351 | −24.816 | 1.00 | 37.51 | 6 | C |
| ATOM | 770 | CG | ASN | A | 117 | 26.686 | −12.059 | −26.199 | 1.00 | 38.58 | 6 | C |
| ATOM | 771 | OD1 | ASN | A | 117 | 25.471 | −12.017 | −26.389 | 1.00 | 40.21 | 8 | O |
| ATOM | 772 | ND2 | ASN | A | 117 | 27.571 | −11.839 | −27.169 | 1.00 | 40.37 | 7 | N |
| ATOM | 773 | C | ASN | A | 117 | 26.895 | −12.111 | −22.320 | 1.00 | 36.55 | 6 | C |
| ATOM | 774 | O | ASN | A | 117 | 26.206 | −12.907 | −21.671 | 1.00 | 36.33 | 8 | O |
| ATOM | 775 | N | ALA | A | 118 | 28.046 | −11.630 | −21.865 | 1.00 | 35.80 | 7 | N |
| ATOM | 776 | CA | ALA | A | 118 | 28.604 | −12.066 | −20.591 | 1.00 | 35.11 | 6 | C |
| ATOM | 777 | CB | ALA | A | 118 | 30.063 | −11.596 | −20.465 | 1.00 | 35.22 | 6 | C |
| ATOM | 778 | C | ALA | A | 118 | 27.786 | −11.594 | −19.385 | 1.00 | 34.02 | 6 | C |
| ATOM | 779 | O | ALA | A | 118 | 27.867 | −12.181 | −18.303 | 1.00 | 33.55 | 8 | O |
| ATOM | 780 | N | LEU | A | 119 | 26.972 | −10.565 | −19.587 | 1.00 | 33.40 | 7 | N |
| ATOM | 781 | CA | LEU | A | 119 | 26.227 | −9.942 | −18.491 | 1.00 | 33.05 | 6 | C |
| ATOM | 782 | CB | LEU | A | 119 | 26.328 | −8.416 | −18.609 | 1.00 | 33.25 | 6 | C |
| ATOM | 783 | CG | LEU | A | 119 | 27.690 | −7.786 | −18.323 | 1.00 | 34.22 | 6 | C |
| ATOM | 784 | CD1 | LEU | A | 119 | 27.621 | −6.275 | −18.502 | 1.00 | 35.25 | 6 | C |
| ATOM | 785 | CD2 | LEU | A | 119 | 28.119 | −8.137 | −16.917 | 1.00 | 36.37 | 6 | C |
| ATOM | 786 | C | LEU | A | 119 | 24.745 | −10.322 | −18.391 | 1.00 | 32.71 | 6 | C |
| ATOM | 787 | O | LEU | A | 119 | 24.071 | −9.939 | −17.436 | 1.00 | 31.84 | 8 | O |
| ATOM | 788 | N | MET | A | 120 | 24.234 | −11.080 | −19.356 | 1.00 | 32.22 | 7 | N |
| ATOM | 789 | CA | MET | A | 120 | 22.806 | −11.400 | −19.362 | 1.00 | 31.92 | 6 | C |
| ATOM | 790 | CB | MET | A | 120 | 22.444 | −12.265 | −20.567 | 1.00 | 32.59 | 6 | C |
| ATOM | 791 | CG | MET | A | 120 | 22.717 | −11.571 | −21.905 | 1.00 | 34.09 | 6 | C |
| ATOM | 792 | SD | MET | A | 120 | 22.013 | −9.907 | −22.079 | 1.00 | 38.77 | 16 | S |
| ATOM | 793 | CE | MET | A | 120 | 20.285 | −10.247 | −21.682 | 1.00 | 38.80 | 6 | C |
| ATOM | 794 | C | MET | A | 120 | 22.259 | −11.979 | −18.042 | 1.00 | 31.30 | 6 | C |
| ATOM | 795 | O | MET | A | 120 | 21.224 | −11.531 | −17.558 | 1.00 | 30.96 | 8 | O |
| ATOM | 796 | N | LYS | A | 121 | 22.960 | −12.934 | −17.441 | 1.00 | 30.40 | 7 | N |
| ATOM | 797 | CA | LYS | A | 121 | 22.510 | −13.503 | −16.167 | 1.00 | 30.26 | 6 | C |
| ATOM | 798 | CB | LYS | A | 121 | 23.373 | −14.696 | −15.761 | 1.00 | 30.27 | 6 | C |
| ATOM | 799 | CG | LYS | A | 121 | 23.150 | −15.967 | −16.604 | 1.00 | 33.46 | 6 | C |
| ATOM | 800 | CD | LYS | A | 121 | 23.970 | −17.127 | −16.066 | 1.00 | 35.55 | 6 | C |
| ATOM | 801 | CE | LYS | A | 121 | 23.753 | −18.400 | −16.889 | 1.00 | 38.65 | 6 | C |
| ATOM | 802 | NZ | LYS | A | 121 | 23.433 | −18.087 | −18.308 | 1.00 | 39.64 | 7 | N |
| ATOM | 803 | C | LYS | A | 121 | 22.513 | −12.456 | −15.025 | 1.00 | 29.34 | 6 | C |
| ATOM | 804 | O | LYS | A | 121 | 21.611 | −12.425 | −14.180 | 1.00 | 28.21 | 8 | O |
| ATOM | 805 | N | LYS | A | 122 | 23.540 | −11.623 | −14.989 | 1.00 | 28.61 | 7 | N |
| ATOM | 806 | CA | LYS | A | 122 | 23.610 | −10.593 | −13.951 | 1.00 | 28.77 | 6 | C |
| ATOM | 807 | CB | LYS | A | 122 | 25.008 | −9.966 | −13.893 | 1.00 | 28.40 | 6 | C |
| ATOM | 808 | CG | LYS | A | 122 | 26.040 | −10.890 | −13.202 | 1.00 | 28.80 | 6 | C |
| ATOM | 809 | CD | LYS | A | 122 | 27.469 | −10.367 | −13.341 | 1.00 | 27.89 | 6 | C |
| ATOM | 810 | CE | LYS | A | 122 | 28.417 | −11.049 | −12.351 | 1.00 | 29.28 | 6 | C |
| ATOM | 811 | NZ | LYS | A | 122 | 29.821 | −10.547 | −12.489 | 1.00 | 27.94 | 7 | N |
| ATOM | 812 | C | LYS | A | 122 | 22.502 | −9.558 | −14.155 | 1.00 | 28.77 | 6 | C |
| ATOM | 813 | O | LYS | A | 122 | 21.872 | −9.110 | −13.192 | 1.00 | 28.75 | 8 | O |
| ATOM | 814 | N | ILE | A | 123 | 22.247 | −9.199 | −15.408 | 1.00 | 28.79 | 7 | N |
| ATOM | 815 | CA | ILE | A | 123 | 21.157 | −8.279 | −15.725 | 1.00 | 29.61 | 6 | C |
| ATOM | 816 | CB | ILE | A | 123 | 21.107 | −8.009 | −17.237 | 1.00 | 29.77 | 6 | C |
| ATOM | 817 | CG1 | ILE | A | 123 | 22.237 | −7.059 | −17.646 | 1.00 | 30.44 | 6 | C |
| ATOM | 818 | CD1 | ILE | A | 123 | 22.525 | −7.043 | −19.147 | 1.00 | 30.78 | 6 | C |
| ATOM | 819 | CG2 | ILE | A | 123 | 19.744 | −7.442 | −17.629 | 1.00 | 30.39 | 6 | C |
| ATOM | 820 | C | ILE | A | 123 | 19.807 | −8.833 | −15.256 | 1.00 | 29.86 | 6 | C |
| ATOM | 821 | O | ILE | A | 123 | 18.965 | −8.097 | −14.733 | 1.00 | 29.63 | 8 | O |
| ATOM | 822 | N | THR | A | 124 | 19.603 | −10.135 | −15.442 | 1.00 | 30.05 | 7 | N |
| ATOM | 823 | CA | THR | A | 124 | 18.347 | −10.774 | −15.045 | 1.00 | 30.32 | 6 | C |
| ATOM | 824 | CB | THR | A | 124 | 18.306 | −12.235 | −15.529 | 1.00 | 30.81 | 6 | C |
| ATOM | 825 | OG1 | THR | A | 124 | 18.216 | −12.258 | −16.963 | 1.00 | 31.11 | 8 | O |
| ATOM | 826 | CG2 | THR | A | 124 | 17.012 | −12.909 | −15.086 | 1.00 | 31.08 | 6 | C |
| ATOM | 827 | C | THR | A | 124 | 18.141 | −10.705 | −13.544 | 1.00 | 30.49 | 6 | C |
| ATOM | 828 | O | THR | A | 124 | 17.042 | −10.417 | −13.062 | 1.00 | 29.67 | 8 | O |
| ATOM | 829 | N | LEU | A | 125 | 19.212 | −10.964 | −12.807 | 1.00 | 30.74 | 7 | N |
| ATOM | 830 | CA | LEU | A | 125 | 19.172 | −10.897 | −11.356 | 1.00 | 31.48 | 6 | C |
| ATOM | 831 | CB | LEU | A | 125 | 20.505 | −11.357 | −10.780 | 1.00 | 31.93 | 6 | C |
| ATOM | 832 | CG | LEU | A | 125 | 20.591 | −12.868 | −10.563 | 1.00 | 34.56 | 6 | C |
| ATOM | 833 | CD1 | LEU | A | 125 | 22.040 | −13.324 | −10.481 | 1.00 | 37.59 | 6 | C |
| ATOM | 834 | CD2 | LEU | A | 125 | 19.841 | −13.214 | −9.287 | 1.00 | 36.58 | 6 | C |
| ATOM | 835 | C | LEU | A | 125 | 18.872 | −9.475 | −10.893 | 1.00 | 31.05 | 6 | C |
| ATOM | 836 | O | LEU | A | 125 | 18.117 | −9.273 | −9.945 | 1.00 | 31.71 | 8 | O |
| ATOM | 837 | N | LEU | A | 126 | 19.472 | −8.494 | −11.554 | 1.00 | 30.76 | 7 | N |
| ATOM | 838 | CA | LEU | A | 126 | 19.265 | −7.098 | −11.173 | 1.00 | 30.69 | 6 | C |
| ATOM | 839 | CB | LEU | A | 126 | 20.215 | −6.172 | −11.936 | 1.00 | 30.65 | 6 | C |
| ATOM | 840 | CG | LEU | A | 126 | 20.182 | −4.691 | −11.530 | 1.00 | 30.81 | 6 | C |
| ATOM | 841 | CD1 | LEU | A | 126 | 20.007 | −4.521 | −10.014 | 1.00 | 31.78 | 6 | C |
| ATOM | 842 | CD2 | LEU | A | 126 | 21.431 | −3.961 | −12.006 | 1.00 | 30.87 | 6 | C |
| ATOM | 843 | C | LEU | A | 126 | 17.815 | −6.694 | −11.397 | 1.00 | 31.15 | 6 | C |
| ATOM | 844 | O | LEU | A | 126 | 17.205 | −6.038 | −10.552 | 1.00 | 30.24 | 8 | O |
| ATOM | 845 | N | LYS | A | 127 | 17.256 | −7.090 | −12.538 | 1.00 | 31.51 | 7 | N |
| ATOM | 846 | CA | LYS | A | 127 | 15.858 | −6.790 | −12.814 | 1.00 | 32.55 | 6 | C |
| ATOM | 847 | CB | LYS | A | 127 | 15.478 | −7.183 | −14.238 | 1.00 | 32.83 | 6 | C |
| ATOM | 848 | CG | LYS | A | 127 | 15.983 | −6.183 | −15.249 | 1.00 | 35.21 | 6 | C |
| ATOM | 849 | CD | LYS | A | 127 | 15.786 | −6.665 | −16.663 | 1.00 | 38.59 | 6 | C |
| ATOM | 850 | CE | LYS | A | 127 | 16.504 | −5.741 | −17.630 | 1.00 | 40.76 | 6 | C |
| ATOM | 851 | NZ | LYS | A | 127 | 15.590 | −4.779 | −18.294 | 1.00 | 42.33 | 7 | N |
| ATOM | 852 | C | LYS | A | 127 | 14.944 | −7.442 | −11.786 | 1.00 | 32.75 | 6 | C |
| ATOM | 853 | O | LYS | A | 127 | 13.950 | −6.842 | −11.365 | 1.00 | 32.80 | 8 | O |
| ATOM | 854 | N | TYR | A | 128 | 15.268 | −8.662 | −11.372 | 1.00 | 33.10 | 7 | N |
| ATOM | 855 | CA | TYR | A | 128 | 14.495 | −9.284 | −10.298 | 1.00 | 33.78 | 6 | C |
| ATOM | 856 | CB | TYR | A | 128 | 15.046 | −10.647 | −9.904 | 1.00 | 34.29 | 6 | C |
| ATOM | 857 | CG | TYR | A | 128 | 14.322 | −11.233 | −8.714 | 1.00 | 37.18 | 6 | C |
| ATOM | 858 | CD1 | TYR | A | 128 | 14.931 | −11.307 | −7.474 | 1.00 | 40.84 | 6 | C |
| ATOM | 859 | CE1 | TYR | A | 128 | 14.270 | −11.835 | −6.382 | 1.00 | 42.52 | 6 | C |
| ATOM | 860 | CZ | TYR | A | 128 | 12.984 | −12.297 | −6.518 | 1.00 | 43.44 | 6 | C |
| ATOM | 861 | OH | TYR | A | 128 | 12.340 | −12.821 | −5.419 | 1.00 | 46.68 | 8 | O |
| ATOM | 862 | CE2 | TYR | A | 128 | 12.347 | −12.235 | −7.736 | 1.00 | 42.86 | 6 | C |
| ATOM | 863 | CD2 | TYR | A | 128 | 13.016 | −11.700 | −8.829 | 1.00 | 40.68 | 6 | C |
| ATOM | 864 | C | TYR | A | 128 | 14.497 | −8.385 | −9.059 | 1.00 | 33.36 | 6 | C |
| ATOM | 865 | O | TYR | A | 128 | 13.445 | −8.136 | −8.469 | 1.00 | 32.70 | 8 | O |
| ATOM | 866 | N | PHE | A | 129 | 15.683 | −7.922 | −8.659 | 1.00 | 33.07 | 7 | N |
| ATOM | 867 | CA | PHE | A | 129 | 15.797 | −7.020 | −7.501 | 1.00 | 33.01 | 6 | C |
| ATOM | 868 | CB | PHE | A | 129 | 17.255 | −6.609 | −7.244 | 1.00 | 33.14 | 6 | C |
| ATOM | 869 | CG | PHE | A | 129 | 18.074 | −7.648 | −6.523 | 1.00 | 34.24 | 6 | C |
| ATOM | 870 | CD1 | PHE | A | 129 | 19.076 | −8.325 | −7.178 | 1.00 | 35.93 | 6 | C |
| ATOM | 871 | CE1 | PHE | A | 129 | 19.839 | −9.288 | −6.523 | 1.00 | 36.77 | 6 | C |
| ATOM | 872 | CZ | PHE | A | 129 | 19.611 | −9.552 | −5.183 | 1.00 | 37.12 | 6 | C |
| ATOM | 873 | CE2 | PHE | A | 129 | 18.624 | −8.859 | −4.505 | 1.00 | 36.61 | 6 | C |
| ATOM | 874 | CD2 | PHE | A | 129 | 17.862 | −7.914 | −5.177 | 1.00 | 36.22 | 6 | C |
| ATOM | 875 | C | PHE | A | 129 | 14.956 | −5.762 | −7.707 | 1.00 | 32.81 | 6 | C |
| ATOM | 876 | O | PHE | A | 129 | 14.213 | −5.347 | −6.812 | 1.00 | 32.43 | 8 | O |
| ATOM | 877 | N | ARG | A | 130 | 15.090 | −5.144 | −8.876 | 1.00 | 32.83 | 7 | N |
| ATOM | 878 | CA | ARG | A | 130 | 14.347 | −3.922 | −9.173 | 1.00 | 33.47 | 6 | C |
| ATOM | 879 | CB | ARG | A | 130 | 14.625 | −3.434 | −10.595 | 1.00 | 33.52 | 6 | C |
| ATOM | 880 | CG | ARG | A | 130 | 13.696 | −2.287 | −11.010 | 1.00 | 34.77 | 6 | C |
| ATOM | 881 | CD | ARG | A | 130 | 13.624 | −2.022 | −12.500 | 1.00 | 36.82 | 6 | C |
| ATOM | 882 | NE | ARG | A | 130 | 13.117 | −3.171 | −13.245 | 1.00 | 37.03 | 7 | N |
| ATOM | 883 | CZ | ARG | A | 130 | 13.233 | −3.298 | −14.557 | 1.00 | 38.07 | 6 | C |
| ATOM | 884 | NH1 | ARG | A | 130 | 13.833 | −2.344 | −15.253 | 1.00 | 39.49 | 7 | N |
| ATOM | 885 | NH2 | ARG | A | 130 | 12.755 | −4.370 | −15.174 | 1.00 | 37.52 | 7 | N |
| ATOM | 886 | C | ARG | A | 130 | 12.845 | −4.140 | −9.000 | 1.00 | 33.31 | 6 | C |
| ATOM | 887 | O | ARG | A | 130 | 12.151 | −3.328 | −8.386 | 1.00 | 32.73 | 8 | O |
| ATOM | 888 | N | ASN | A | 131 | 12.352 | −5.245 | −9.548 | 1.00 | 33.24 | 7 | N |
| ATOM | 889 | CA | ASN | A | 131 | 10.933 | −5.561 | −9.480 | 1.00 | 33.80 | 6 | C |
| ATOM | 890 | CB | ASN | A | 131 | 10.596 | −6.732 | −10.406 | 1.00 | 33.96 | 6 | C |
| ATOM | 891 | CG | ASN | A | 131 | 10.819 | −6.399 | −11.867 | 1.00 | 35.72 | 6 | C |
| ATOM | 892 | OD1 | ASN | A | 131 | 10.921 | −5.229 | −12.244 | 1.00 | 38.11 | 8 | O |
| ATOM | 893 | ND2 | ASN | A | 131 | 10.893 | −7.428 | −12.702 | 1.00 | 36.73 | 7 | N |
| ATOM | 894 | C | ASN | A | 131 | 10.486 | −5.870 | −8.061 | 1.00 | 33.52 | 6 | C |
| ATOM | 895 | O | ASN | A | 131 | 9.419 | −5.433 | −7.640 | 1.00 | 33.38 | 8 | O |
| ATOM | 896 | N | TYR | A | 132 | 11.305 | −6.617 | −7.321 | 1.00 | 33.15 | 7 | N |
| ATOM | 897 | CA | TYR | A | 132 | 10.978 | −6.933 | −5.938 | 1.00 | 33.32 | 6 | C |
| ATOM | 898 | CB | TYR | A | 132 | 12.040 | −7.850 | −5.323 | 1.00 | 33.61 | 6 | C |
| ATOM | 899 | CG | TYR | A | 132 | 11.686 | −8.294 | −3.923 | 1.00 | 35.78 | 6 | C |
| ATOM | 900 | CD1 | TYR | A | 132 | 11.066 | −9.515 | −3.700 | 1.00 | 36.19 | 6 | C |
| ATOM | 901 | CE1 | TYR | A | 132 | 10.727 | −9.920 | −2.422 | 1.00 | 38.23 | 6 | C |
| ATOM | 902 | CZ | TYR | A | 132 | 11.009 | −9.103 | −1.352 | 1.00 | 38.11 | 6 | C |
| ATOM | 903 | OH | TYR | A | 132 | 10.674 | −9.504 | −0.077 | 1.00 | 39.25 | 8 | O |
| ATOM | 904 | CE2 | TYR | A | 132 | 11.623 | −7.886 | −1.548 | 1.00 | 37.39 | 6 | C |
| ATOM | 905 | CD2 | TYR | A | 132 | 11.958 | −7.487 | −2.825 | 1.00 | 36.22 | 6 | C |
| ATOM | 906 | C | TYR | A | 132 | 10.849 | −5.658 | −5.094 | 1.00 | 33.10 | 6 | C |
| ATOM | 907 | O | TYR | A | 132 | 9.887 | −5.483 | −4.336 | 1.00 | 32.61 | 8 | O |
| ATOM | 908 | N | MET | A | 133 | 11.821 | −4.766 | −5.236 | 1.00 | 32.43 | 7 | N |
| ATOM | 909 | CA | MET | A | 133 | 11.833 | −3.533 | −4.457 | 1.00 | 32.77 | 6 | C |
| ATOM | 910 | CB | MET | A | 133 | 13.163 | −2.802 | −4.644 | 1.00 | 32.05 | 6 | C |
| ATOM | 911 | CG | MET | A | 133 | 14.345 | −3.557 | −4.063 | 1.00 | 30.87 | 6 | C |
| ATOM | 912 | SD | MET | A | 133 | 15.894 | −2.591 | −4.095 | 1.00 | 29.32 | 16 | S |
| ATOM | 913 | CE | MET | A | 133 | 16.221 | −2.588 | −5.846 | 1.00 | 28.38 | 6 | C |
| ATOM | 914 | C | MET | A | 133 | 10.659 | −2.621 | −4.802 | 1.00 | 33.03 | 6 | C |
| ATOM | 915 | O | MET | A | 133 | 10.058 | −2.009 | −3.921 | 1.00 | 32.77 | 8 | O |
| ATOM | 916 | N | SER | A | 134 | 10.334 | −2.538 | −6.086 | 1.00 | 33.87 | 7 | N |
| ATOM | 917 | CA | SER | A | 134 | 9.218 | −1.717 | −6.533 | 1.00 | 35.17 | 6 | C |
| ATOM | 918 | CB | SER | A | 134 | 9.214 | −1.615 | −8.059 | 1.00 | 35.68 | 6 | C |
| ATOM | 919 | OG | SER | A | 134 | 7.952 | −1.180 | −8.534 | 1.00 | 37.31 | 8 | O |
| ATOM | 920 | C | SER | A | 134 | 7.867 | −2.237 | −6.021 | 1.00 | 35.37 | 6 | C |
| ATOM | 921 | O | SER | A | 134 | 6.973 | −1.453 | −5.694 | 1.00 | 35.81 | 8 | O |
| ATOM | 922 | N | GLU | A | 135 | 7.719 | −3.553 | −5.934 | 1.00 | 35.53 | 7 | N |
| ATOM | 923 | CA | GLU | A | 135 | 6.452 | −4.133 | −5.486 | 1.00 | 35.74 | 6 | C |
| ATOM | 924 | CB | GLU | A | 135 | 6.284 | −5.545 | −6.052 | 1.00 | 36.44 | 6 | C |
| ATOM | 925 | CG | GLU | A | 135 | 6.145 | −5.599 | −7.567 | 1.00 | 39.80 | 6 | C |
| ATOM | 926 | CD | GLU | A | 135 | 4.701 | −5.532 | −8.032 | 1.00 | 44.09 | 6 | C |
| ATOM | 927 | OE1 | GLU | A | 135 | 3.819 | −5.185 | −7.213 | 1.00 | 45.82 | 8 | O |
| ATOM | 928 | OE2 | GLU | A | 135 | 4.444 | −5.840 | −9.221 | 1.00 | 46.81 | 8 | O |
| ATOM | 929 | C | GLU | A | 135 | 6.251 | −4.171 | −3.967 | 1.00 | 34.96 | 6 | C |
| ATOM | 930 | O | GLU | A | 135 | 5.125 | −4.011 | −3.482 | 1.00 | 34.64 | 8 | O |
| ATOM | 931 | N | HIS | A | 136 | 7.333 | −4.358 | −3.216 | 1.00 | 33.49 | 7 | N |
| ATOM | 932 | CA | HIS | A | 136 | 7.220 | −4.574 | −1.777 | 1.00 | 32.95 | 6 | C |
| ATOM | 933 | CB | HIS | A | 136 | 7.933 | −5.875 | −1.405 | 1.00 | 33.31 | 6 | C |
| ATOM | 934 | CG | HIS | A | 136 | 7.430 | −7.075 | −2.142 | 1.00 | 35.42 | 6 | C |
| ATOM | 935 | ND1 | HIS | A | 136 | 6.323 | −7.787 | −1.735 | 1.00 | 36.88 | 7 | N |
| ATOM | 936 | CE1 | HIS | A | 136 | 6.118 | −8.791 | −2.570 | 1.00 | 38.14 | 6 | C |
| ATOM | 937 | NE2 | HIS | A | 136 | 7.050 | −8.752 | −3.506 | 1.00 | 38.14 | 7 | N |
| ATOM | 938 | CD2 | HIS | A | 136 | 7.884 | −7.688 | −3.261 | 1.00 | 37.14 | 6 | C |
| ATOM | 939 | C | HIS | A | 136 | 7.757 | −3.503 | −0.827 | 1.00 | 31.87 | 6 | C |
| ATOM | 940 | O | HIS | A | 136 | 7.369 | −3.482 | 0.332 | 1.00 | 31.61 | 8 | O |
| ATOM | 941 | N | LEU | A | 137 | 8.643 | −2.630 | −1.297 | 1.00 | 30.88 | 7 | N |
| ATOM | 942 | CA | LEU | A | 137 | 9.362 | −1.751 | −0.361 | 1.00 | 30.11 | 6 | C |
| ATOM | 943 | CB | LEU | A | 137 | 10.871 | −2.021 | −0.444 | 1.00 | 29.63 | 6 | C |
| ATOM | 944 | CG | LEU | A | 137 | 11.300 | −3.497 | −0.339 | 1.00 | 29.18 | 6 | C |
| ATOM | 945 | CD1 | LEU | A | 137 | 12.823 | −3.621 | −0.382 | 1.00 | 27.36 | 6 | C |
| ATOM | 946 | CD2 | LEU | A | 137 | 10.755 | −4.166 | 0.918 | 1.00 | 27.32 | 6 | C |
| ATOM | 947 | C | LEU | A | 137 | 9.108 | −0.254 | −0.468 | 1.00 | 29.92 | 6 | C |
| ATOM | 948 | O | LEU | A | 137 | 8.901 | 0.286 | −1.545 | 1.00 | 29.22 | 8 | O |
| ATOM | 949 | N | LEU | A | 138 | 9.165 | 0.401 | 0.687 | 1.00 | 29.80 | 7 | N |
| ATOM | 950 | CA | LEU | A | 138 | 8.936 | 1.834 | 0.809 | 1.00 | 30.37 | 6 | C |
| ATOM | 951 | CB | LEU | A | 138 | 8.575 | 2.142 | 2.255 | 1.00 | 30.84 | 6 | C |
| ATOM | 952 | CG | LEU | A | 138 | 8.219 | 3.594 | 2.534 | 1.00 | 31.89 | 6 | C |
| ATOM | 953 | CD1 | LEU | A | 138 | 6.971 | 3.986 | 1.732 | 1.00 | 34.04 | 6 | C |
| ATOM | 954 | CD2 | LEU | A | 138 | 7.996 | 3.742 | 4.020 | 1.00 | 35.04 | 6 | C |
| ATOM | 955 | C | LEU | A | 138 | 10.181 | 2.633 | 0.423 | 1.00 | 30.37 | 6 | C |
| ATOM | 956 | O | LEU | A | 138 | 11.286 | 2.272 | 0.805 | 1.00 | 30.44 | 8 | O |
| ATOM | 957 | N | LYS | A | 139 | 10.008 | 3.703 | −0.346 | 1.00 | 30.35 | 7 | N |
| ATOM | 958 | CA | LYS | A | 139 | 11.150 | 4.525 | −0.761 | 1.00 | 30.71 | 6 | C |
| ATOM | 959 | CB | LYS | A | 139 | 10.823 | 5.264 | −2.061 | 1.00 | 30.82 | 6 | C |
| ATOM | 960 | CG | LYS | A | 139 | 11.970 | 6.066 | −2.618 | 1.00 | 31.83 | 6 | C |
| ATOM | 961 | CD | LYS | A | 139 | 11.733 | 6.531 | −4.057 | 1.00 | 32.82 | 6 | C |
| ATOM | 962 | CE | LYS | A | 139 | 12.878 | 7.451 | −4.495 | 1.00 | 32.63 | 6 | C |
| ATOM | 963 | NZ | LYS | A | 139 | 12.797 | 7.904 | −5.908 | 1.00 | 32.89 | 7 | N |
| ATOM | 964 | C | LYS | A | 139 | 11.585 | 5.513 | 0.326 | 1.00 | 30.83 | 6 | C |
| ATOM | 965 | O | LYS | A | 139 | 10.830 | 6.420 | 0.692 | 1.00 | 30.70 | 8 | O |
| ATOM | 966 | N | ALA | A | 140 | 12.797 | 5.333 | 0.846 | 1.00 | 30.65 | 7 | N |
| ATOM | 967 | CA | ALA | A | 140 | 13.313 | 6.226 | 1.877 | 1.00 | 30.81 | 6 | C |
| ATOM | 968 | CB | ALA | A | 140 | 14.529 | 5.619 | 2.574 | 1.00 | 30.74 | 6 | C |
| ATOM | 969 | C | ALA | A | 140 | 13.667 | 7.586 | 1.288 | 1.00 | 31.03 | 6 | C |
| ATOM | 970 | O | ALA | A | 140 | 14.249 | 7.678 | 0.208 | 1.00 | 29.95 | 8 | O |
| ATOM | 971 | N | GLY | A | 141 | 13.302 | 8.642 | 2.010 | 1.00 | 31.87 | 7 | N |
| ATOM | 972 | CA | GLY | A | 141 | 13.600 | 9.989 | 1.568 | 1.00 | 33.39 | 6 | C |
| ATOM | 973 | C | GLY | A | 141 | 12.687 | 10.419 | 0.443 | 1.00 | 34.35 | 6 | C |
| ATOM | 974 | O | GLY | A | 141 | 13.056 | 11.249 | −0.386 | 1.00 | 34.20 | 8 | O |
| ATOM | 975 | N | ALA | A | 142 | 11.490 | 9.852 | 0.421 | 1.00 | 35.76 | 7 | N |
| ATOM | 976 | CA | ALA | A | 142 | 10.509 | 10.176 | −0.606 | 1.00 | 37.44 | 6 | C |
| ATOM | 977 | CB | ALA | A | 142 | 9.254 | 9.343 | −0.410 | 1.00 | 37.52 | 6 | C |
| ATOM | 978 | C | ALA | A | 142 | 10.162 | 11.666 | −0.588 | 1.00 | 38.51 | 6 | C |
| ATOM | 979 | O | ALA | A | 142 | 9.806 | 12.243 | −1.618 | 1.00 | 38.73 | 8 | O |
| ATOM | 980 | N | ASN | A | 143 | 10.265 | 12.282 | 0.585 | 1.00 | 39.78 | 7 | N |
| ATOM | 981 | CA | ASN | A | 143 | 9.947 | 13.702 | 0.729 | 1.00 | 41.20 | 6 | C |
| ATOM | 982 | CB | ASN | A | 143 | 9.266 | 13.962 | 2.076 | 1.00 | 41.20 | 6 | C |
| ATOM | 983 | CG | ASN | A | 143 | 10.130 | 13.547 | 3.257 | 1.00 | 41.87 | 6 | C |
| ATOM | 984 | OD1 | ASN | A | 143 | 11.169 | 12.898 | 3.087 | 1.00 | 42.25 | 8 | O |
| ATOM | 985 | ND2 | ASN | A | 143 | 9.704 | 13.919 | 4.463 | 1.00 | 40.49 | 7 | N |
| ATOM | 986 | C | ASN | A | 143 | 11.163 | 14.611 | 0.581 | 1.00 | 42.02 | 6 | C |
| ATOM | 987 | O | ASN | A | 143 | 11.069 | 15.825 | 0.781 | 1.00 | 42.10 | 8 | O |
| ATOM | 988 | N | ILE | A | 144 | 12.307 | 14.031 | 0.230 | 1.00 | 42.79 | 7 | N |
| ATOM | 989 | CA | ILE | A | 144 | 13.530 | 14.817 | 0.099 | 1.00 | 43.76 | 6 | C |
| ATOM | 990 | CB | ILE | A | 144 | 14.733 | 14.093 | 0.739 | 1.00 | 43.56 | 6 | C |
| ATOM | 991 | CG1 | ILE | A | 144 | 14.392 | 13.605 | 2.150 | 1.00 | 43.51 | 6 | C |
| ATOM | 992 | CD1 | ILE | A | 144 | 15.560 | 12.895 | 2.865 | 1.00 | 44.14 | 6 | C |
| ATOM | 993 | CG2 | ILE | A | 144 | 15.941 | 15.012 | 0.751 | 1.00 | 43.45 | 6 | C |
| ATOM | 994 | C | ILE | A | 144 | 13.858 | 15.124 | −1.351 | 1.00 | 44.78 | 6 | C |
| ATOM | 995 | O | ILE | A | 144 | 13.718 | 14.266 | −2.231 | 1.00 | 44.82 | 8 | O |
| ATOM | 996 | N | THR | A | 145 | 14.306 | 16.352 | −1.588 | 1.00 | 46.00 | 7 | N |
| ATOM | 997 | CA | THR | A | 145 | 14.717 | 16.781 | −2.914 | 1.00 | 47.32 | 6 | C |
| ATOM | 998 | CB | THR | A | 145 | 14.106 | 18.153 | −3.251 | 1.00 | 47.48 | 6 | C |
| ATOM | 999 | OG1 | THR | A | 145 | 12.681 | 18.032 | −3.379 | 1.00 | 47.75 | 8 | O |
| ATOM | 1000 | CG2 | THR | A | 145 | 14.555 | 18.603 | −4.637 | 1.00 | 47.41 | 6 | C |
| ATOM | 1001 | C | THR | A | 145 | 16.236 | 16.870 | −2.948 | 1.00 | 48.35 | 6 | C |
| ATOM | 1002 | O | THR | A | 145 | 16.824 | 17.752 | −2.327 | 1.00 | 47.79 | 8 | O |
| ATOM | 1003 | N | PRO | A | 146 | 16.871 | 15.965 | −3.687 | 1.00 | 49.56 | 7 | N |
| ATOM | 1004 | CA | PRO | A | 146 | 18.334 | 15.916 | −3.748 | 1.00 | 50.70 | 6 | C |
| ATOM | 1005 | CB | PRO | A | 146 | 18.610 | 14.687 | −4.623 | 1.00 | 50.57 | 6 | C |
| ATOM | 1006 | CG | PRO | A | 146 | 17.377 | 14.486 | −5.410 | 1.00 | 50.15 | 6 | C |
| ATOM | 1007 | CD | PRO | A | 146 | 16.236 | 14.963 | −4.561 | 1.00 | 49.76 | 6 | C |
| ATOM | 1008 | C | PRO | A | 146 | 18.870 | 17.157 | −4.433 | 1.00 | 52.00 | 6 | C |
| ATOM | 1009 | O | PRO | A | 146 | 18.140 | 17.784 | −5.203 | 1.00 | 52.03 | 8 | O |
| ATOM | 1010 | N | ARG | A | 147 | 20.114 | 17.525 | −4.155 | 1.00 | 53.52 | 7 | N |
| ATOM | 1011 | CA | ARG | A | 147 | 20.694 | 18.665 | −4.843 | 1.00 | 55.35 | 6 | C |
| ATOM | 1012 | CB | ARG | A | 147 | 21.863 | 19.266 | −4.053 | 1.00 | 55.28 | 6 | C |
| ATOM | 1013 | CG | ARG | A | 147 | 23.214 | 18.590 | −4.231 | 1.00 | 55.08 | 6 | C |
| ATOM | 1014 | CD | ARG | A | 147 | 24.306 | 19.208 | −3.360 | 1.00 | 54.57 | 6 | C |
| ATOM | 1015 | NE | ARG | A | 147 | 25.650 | 18.751 | −3.702 | 1.00 | 54.12 | 7 | N |
| ATOM | 1016 | CZ | ARG | A | 147 | 26.310 | 17.808 | −3.044 | 1.00 | 54.07 | 6 | C |
| ATOM | 1017 | NH1 | ARG | A | 147 | 25.750 | 17.199 | −2.008 | 1.00 | 53.40 | 7 | N |
| ATOM | 1018 | NH2 | ARG | A | 147 | 27.533 | 17.464 | −3.424 | 1.00 | 54.71 | 7 | N |
| ATOM | 1019 | C | ARG | A | 147 | 21.114 | 18.207 | −6.235 | 1.00 | 56.73 | 6 | C |
| ATOM | 1020 | O | ARG | A | 147 | 20.917 | 17.047 | −6.597 | 1.00 | 56.81 | 8 | O |
| ATOM | 1021 | N | GLU | A | 148 | 21.667 | 19.114 | −7.028 | 1.00 | 58.51 | 7 | N |
| ATOM | 1022 | CA | GLU | A | 148 | 22.093 | 18.751 | −8.371 | 1.00 | 60.30 | 6 | C |
| ATOM | 1023 | CB | GLU | A | 148 | 21.094 | 19.278 | −9.409 | 1.00 | 60.58 | 6 | C |
| ATOM | 1024 | CG | GLU | A | 148 | 19.741 | 18.573 | −9.355 | 1.00 | 61.94 | 6 | C |
| ATOM | 1025 | CD | GLU | A | 148 | 18.802 | 18.971 | −10.481 | 1.00 | 63.38 | 6 | C |
| ATOM | 1026 | OE1 | GLU | A | 148 | 19.203 | 19.779 | −11.350 | 1.00 | 64.12 | 8 | O |
| ATOM | 1027 | OE2 | GLU | A | 148 | 17.655 | 18.470 | −10.498 | 1.00 | 63.83 | 8 | O |
| ATOM | 1028 | C | GLU | A | 148 | 23.503 | 19.255 | −8.653 | 1.00 | 61.21 | 6 | C |
| ATOM | 1029 | O | GLU | A | 148 | 23.875 | 20.351 | −8.223 | 1.00 | 61.66 | 8 | O |
| ATOM | 1030 | N | GLY | A | 149 | 24.286 | 18.446 | −9.360 | 1.00 | 62.09 | 7 | N |
| ATOM | 1031 | CA | GLY | A | 149 | 25.647 | 18.812 | −9.718 | 1.00 | 62.88 | 6 | C |
| ATOM | 1032 | C | GLY | A | 149 | 26.148 | 18.045 | −10.929 | 1.00 | 63.42 | 6 | C |
| ATOM | 1033 | O | GLY | A | 149 | 26.292 | 16.822 | −10.881 | 1.00 | 63.58 | 8 | O |
| ATOM | 1034 | N | ASP | A | 150 | 26.421 | 18.765 | −12.015 | 1.00 | 63.96 | 7 | N |
| ATOM | 1035 | CA | ASP | A | 150 | 26.880 | 18.152 | −13.261 | 1.00 | 64.35 | 6 | C |
| ATOM | 1036 | CB | ASP | A | 150 | 28.190 | 17.388 | −13.049 | 1.00 | 64.61 | 6 | C |
| ATOM | 1037 | CG | ASP | A | 150 | 29.268 | 18.247 | −12.419 | 1.00 | 65.54 | 6 | C |
| ATOM | 1038 | OD1 | ASP | A | 150 | 29.231 | 19.484 | −12.603 | 1.00 | 66.88 | 8 | O |
| ATOM | 1039 | OD2 | ASP | A | 150 | 30.189 | 17.775 | −11.719 | 1.00 | 66.50 | 8 | O |
| ATOM | 1040 | C | ASP | A | 150 | 25.809 | 17.226 | −13.838 | 1.00 | 64.29 | 6 | C |
| ATOM | 1041 | O | ASP | A | 150 | 24.663 | 17.639 | −14.015 | 1.00 | 64.48 | 8 | O |
| ATOM | 1042 | N | GLU | A | 151 | 26.192 | 15.982 | −14.122 | 1.00 | 64.02 | 7 | N |
| ATOM | 1043 | CA | GLU | A | 151 | 25.292 | 14.969 | −14.684 | 1.00 | 63.67 | 6 | C |
| ATOM | 1044 | CB | GLU | A | 151 | 24.029 | 15.603 | −15.288 | 1.00 | 63.88 | 6 | C |
| ATOM | 1045 | CG | GLU | A | 151 | 22.862 | 15.768 | −14.322 | 1.00 | 64.91 | 6 | C |
| ATOM | 1046 | CD | GLU | A | 151 | 21.638 | 16.391 | −14.979 | 1.00 | 66.57 | 6 | C |
| ATOM | 1047 | OE1 | GLU | A | 151 | 21.428 | 16.155 | −16.190 | 1.00 | 66.99 | 8 | O |
| ATOM | 1048 | OE2 | GLU | A | 151 | 20.882 | 17.113 | −14.288 | 1.00 | 67.29 | 8 | O |
| ATOM | 1049 | C | GLU | A | 151 | 26.001 | 14.137 | −15.752 | 1.00 | 63.01 | 6 | C |
| ATOM | 1050 | O | GLU | A | 151 | 25.558 | 13.040 | −16.094 | 1.00 | 63.25 | 8 | O |
| ATOM | 1051 | N | LEU | A | 152 | 27.110 | 14.662 | −16.267 | 1.00 | 61.93 | 7 | N |
| ATOM | 1052 | CA | LEU | A | 152 | 27.850 | 14.017 | −17.354 | 1.00 | 60.75 | 6 | C |
| ATOM | 1053 | CB | LEU | A | 152 | 28.805 | 15.023 | −18.002 | 1.00 | 61.03 | 6 | C |
| ATOM | 1054 | CG | LEU | A | 152 | 28.167 | 16.369 | −18.359 | 1.00 | 61.49 | 6 | C |
| ATOM | 1055 | CD1 | LEU | A | 152 | 29.199 | 17.326 | −18.939 | 1.00 | 62.25 | 6 | C |
| ATOM | 1056 | CD2 | LEU | A | 152 | 27.006 | 16.180 | −19.326 | 1.00 | 62.22 | 6 | C |
| ATOM | 1057 | C | LEU | A | 152 | 28.604 | 12.737 | −16.961 | 1.00 | 59.54 | 6 | C |
| ATOM | 1058 | O | LEU | A | 152 | 28.966 | 11.939 | −17.826 | 1.00 | 59.82 | 8 | O |
| ATOM | 1059 | N | ALA | A | 153 | 28.842 | 12.554 | −15.664 | 1.00 | 57.73 | 7 | N |
| ATOM | 1060 | CA | ALA | A | 153 | 29.528 | 11.370 | −15.140 | 1.00 | 55.74 | 6 | C |
| ATOM | 1061 | CB | ALA | A | 153 | 31.002 | 11.395 | −15.533 | 1.00 | 55.94 | 6 | C |
| ATOM | 1062 | C | ALA | A | 153 | 29.389 | 11.389 | −13.624 | 1.00 | 54.05 | 6 | C |
| ATOM | 1063 | O | ALA | A | 153 | 30.163 | 12.071 | −12.951 | 1.00 | 54.29 | 8 | O |
| ATOM | 1064 | N | ARG | A | 154 | 28.433 | 10.642 | −13.065 | 1.00 | 51.55 | 7 | N |
| ATOM | 1065 | CA | ARG | A | 154 | 28.196 | 10.803 | −11.632 | 1.00 | 48.72 | 6 | C |
| ATOM | 1066 | CB | ARG | A | 154 | 27.585 | 12.188 | −11.413 | 1.00 | 48.98 | 6 | C |
| ATOM | 1067 | CG | ARG | A | 154 | 26.314 | 12.428 | −12.218 | 1.00 | 49.65 | 6 | C |
| ATOM | 1068 | CD | ARG | A | 154 | 25.262 | 11.353 | −12.036 | 1.00 | 50.74 | 6 | C |
| ATOM | 1069 | NE | ARG | A | 154 | 23.946 | 11.781 | −12.481 | 1.00 | 52.59 | 7 | N |
| ATOM | 1070 | CZ | ARG | A | 154 | 22.901 | 10.977 | −12.581 | 1.00 | 53.47 | 6 | C |
| ATOM | 1071 | NH1 | ARG | A | 154 | 23.016 | 9.695 | −12.263 | 1.00 | 55.62 | 7 | N |
| ATOM | 1072 | NH2 | ARG | A | 154 | 21.737 | 11.452 | −12.993 | 1.00 | 53.86 | 7 | N |
| ATOM | 1073 | C | ARG | A | 154 | 27.355 | 9.811 | −10.815 | 1.00 | 46.34 | 6 | C |
| ATOM | 1074 | O | ARG | A | 154 | 26.956 | 10.148 | −9.704 | 1.00 | 46.32 | 8 | O |
| ATOM | 1075 | N | LEU | A | 155 | 27.064 | 8.619 | −11.318 | 1.00 | 42.78 | 7 | N |
| ATOM | 1076 | CA | LEU | A | 155 | 26.300 | 7.677 | −10.496 | 1.00 | 39.40 | 6 | C |
| ATOM | 1077 | CB | LEU | A | 155 | 25.388 | 6.807 | −11.360 | 1.00 | 39.89 | 6 | C |
| ATOM | 1078 | CG | LEU | A | 155 | 24.122 | 6.271 | −10.690 | 1.00 | 40.23 | 6 | C |
| ATOM | 1079 | CD1 | LEU | A | 155 | 23.327 | 7.409 | −10.101 | 1.00 | 41.05 | 6 | C |
| ATOM | 1080 | CD2 | LEU | A | 155 | 23.259 | 5.508 | −11.679 | 1.00 | 40.15 | 6 | C |
| ATOM | 1081 | C | LEU | A | 155 | 27.262 | 6.804 | −9.683 | 1.00 | 36.59 | 6 | C |
| ATOM | 1082 | O | LEU | A | 155 | 28.112 | 6.130 | −10.249 | 1.00 | 35.75 | 8 | O |
| ATOM | 1083 | N | PRO | A | 156 | 27.140 | 6.825 | −8.358 | 1.00 | 33.97 | 7 | N |
| ATOM | 1084 | CA | PRO | A | 156 | 28.030 | 6.030 | −7.504 | 1.00 | 32.21 | 6 | C |
| ATOM | 1085 | CB | PRO | A | 156 | 27.895 | 6.711 | −6.137 | 1.00 | 32.09 | 6 | C |
| ATOM | 1086 | CG | PRO | A | 156 | 26.505 | 7.245 | −6.128 | 1.00 | 33.10 | 6 | C |
| ATOM | 1087 | CD | PRO | A | 156 | 26.175 | 7.614 | −7.569 | 1.00 | 33.75 | 6 | C |
| ATOM | 1088 | C | PRO | A | 156 | 27.543 | 4.590 | −7.382 | 1.00 | 30.42 | 6 | C |
| ATOM | 1089 | O | PRO | A | 156 | 26.371 | 4.322 | −7.627 | 1.00 | 29.50 | 8 | O |
| ATOM | 1090 | N | TYR | A | 157 | 28.435 | 3.671 | −7.017 | 1.00 | 28.48 | 7 | N |
| ATOM | 1091 | CA | TYR | A | 157 | 28.012 | 2.297 | −6.793 | 1.00 | 27.62 | 6 | C |
| ATOM | 1092 | CB | TYR | A | 157 | 28.701 | 1.325 | −7.750 | 1.00 | 27.64 | 6 | C |
| ATOM | 1093 | CG | TYR | A | 157 | 30.199 | 1.396 | −7.711 | 1.00 | 28.30 | 6 | C |
| ATOM | 1094 | CD1 | TYR | A | 157 | 30.927 | 0.684 | −6.762 | 1.00 | 28.82 | 6 | C |
| ATOM | 1095 | CE1 | TYR | A | 157 | 32.308 | 0.759 | −6.724 | 1.00 | 30.62 | 6 | C |
| ATOM | 1096 | CZ | TYR | A | 157 | 32.966 | 1.538 | −7.664 | 1.00 | 31.02 | 6 | C |
| ATOM | 1097 | OH | TYR | A | 157 | 34.336 | 1.621 | −7.646 | 1.00 | 33.38 | 8 | O |
| ATOM | 1098 | CE2 | TYR | A | 157 | 32.262 | 2.261 | −8.600 | 1.00 | 29.83 | 6 | C |
| ATOM | 1099 | CD2 | TYR | A | 157 | 30.895 | 2.184 | −8.626 | 1.00 | 30.03 | 6 | C |
| ATOM | 1100 | C | TYR | A | 157 | 28.328 | 1.930 | −5.354 | 1.00 | 26.56 | 6 | C |
| ATOM | 1101 | O | TYR | A | 157 | 29.028 | 2.657 | −4.655 | 1.00 | 25.59 | 8 | O |
| ATOM | 1102 | N | LEU | A | 158 | 27.818 | 0.795 | −4.917 | 1.00 | 26.50 | 7 | N |
| ATOM | 1103 | CA | LEU | A | 158 | 28.028 | 0.361 | −3.549 | 1.00 | 25.95 | 6 | C |
| ATOM | 1104 | CB | LEU | A | 158 | 26.927 | −0.627 | −3.159 | 1.00 | 26.13 | 6 | C |
| ATOM | 1105 | CG | LEU | A | 158 | 26.975 | −1.109 | −1.717 | 1.00 | 24.82 | 6 | C |
| ATOM | 1106 | CD1 | LEU | A | 158 | 26.752 | 0.064 | −0.751 | 1.00 | 25.10 | 6 | C |
| ATOM | 1107 | CD2 | LEU | A | 158 | 25.919 | −2.197 | −1.532 | 1.00 | 24.37 | 6 | C |
| ATOM | 1108 | C | LEU | A | 158 | 29.413 | −0.286 | −3.418 | 1.00 | 26.52 | 6 | C |
| ATOM | 1109 | O | LEU | A | 158 | 29.679 | −1.318 | −4.025 | 1.00 | 26.29 | 8 | O |
| ATOM | 1110 | N | ARG | A | 159 | 30.298 | 0.339 | −2.648 | 1.00 | 26.73 | 7 | N |
| ATOM | 1111 | CA | ARG | A | 159 | 31.658 | −0.176 | −2.451 | 1.00 | 27.63 | 6 | C |
| ATOM | 1112 | CB | ARG | A | 159 | 32.561 | 0.902 | −1.854 | 1.00 | 28.30 | 6 | C |
| ATOM | 1113 | CG | ARG | A | 159 | 33.108 | 1.914 | −2.848 | 1.00 | 31.65 | 6 | C |
| ATOM | 1114 | CD | ARG | A | 159 | 34.205 | 2.815 | −2.253 | 1.00 | 36.75 | 6 | C |
| ATOM | 1115 | NE | ARG | A | 159 | 34.803 | 3.710 | −3.249 | 1.00 | 40.48 | 7 | N |
| ATOM | 1116 | CZ | ARG | A | 159 | 35.301 | 4.920 | −2.979 | 1.00 | 42.21 | 6 | C |
| ATOM | 1117 | NH1 | ARG | A | 159 | 35.282 | 5.406 | −1.737 | 1.00 | 41.23 | 7 | N |
| ATOM | 1118 | NH2 | ARG | A | 159 | 35.818 | 5.649 | −3.960 | 1.00 | 44.20 | 7 | N |
| ATOM | 1119 | C | ARG | A | 159 | 31.672 | −1.372 | −1.513 | 1.00 | 27.88 | 6 | C |
| ATOM | 1120 | O | ARG | A | 159 | 32.331 | −2.383 | −1.771 | 1.00 | 27.60 | 8 | O |
| ATOM | 1121 | N | THR | A | 160 | 30.967 | −1.234 | −0.396 | 1.00 | 27.42 | 7 | N |
| ATOM | 1122 | CA | THR | A | 160 | 30.840 | −2.330 | 0.557 | 1.00 | 28.18 | 6 | C |
| ATOM | 1123 | CB | THR | A | 160 | 32.181 | −2.595 | 1.292 | 1.00 | 28.55 | 6 | C |
| ATOM | 1124 | OG1 | THR | A | 160 | 32.102 | −3.825 | 2.033 | 1.00 | 31.68 | 8 | O |
| ATOM | 1125 | CG2 | THR | A | 160 | 32.441 | −1.542 | 2.352 | 1.00 | 30.45 | 6 | C |
| ATOM | 1126 | C | THR | A | 160 | 29.721 | −1.994 | 1.535 | 1.00 | 27.01 | 6 | C |
| ATOM | 1127 | O | THR | A | 160 | 29.190 | −0.872 | 1.542 | 1.00 | 26.02 | 8 | O |
| ATOM | 1128 | N | TRP | A | 161 | 29.360 | −2.974 | 2.345 | 1.00 | 26.52 | 7 | N |
| ATOM | 1129 | CA | TRP | A | 161 | 28.300 | −2.817 | 3.325 | 1.00 | 25.88 | 6 | C |
| ATOM | 1130 | CB | TRP | A | 161 | 26.932 | −2.972 | 2.657 | 1.00 | 25.89 | 6 | C |
| ATOM | 1131 | CG | TRP | A | 161 | 26.714 | −4.349 | 2.082 | 1.00 | 26.34 | 6 | C |
| ATOM | 1132 | CD1 | TRP | A | 161 | 27.141 | −4.805 | 0.865 | 1.00 | 27.76 | 6 | C |
| ATOM | 1133 | NE1 | TRP | A | 161 | 26.775 | −6.119 | 0.694 | 1.00 | 27.89 | 7 | N |
| ATOM | 1134 | CE2 | TRP | A | 161 | 26.093 | −6.542 | 1.803 | 1.00 | 29.07 | 6 | C |
| ATOM | 1135 | CD2 | TRP | A | 161 | 26.036 | −5.453 | 2.702 | 1.00 | 28.34 | 6 | C |
| ATOM | 1136 | CE3 | TRP | A | 161 | 25.390 | −5.635 | 3.928 | 1.00 | 29.01 | 6 | C |
| ATOM | 1137 | CZ3 | TRP | A | 161 | 24.820 | −6.868 | 4.209 | 1.00 | 30.77 | 6 | C |
| ATOM | 1138 | CH2 | TRP | A | 161 | 24.888 | −7.928 | 3.292 | 1.00 | 30.47 | 6 | C |
| ATOM | 1139 | CZ2 | TRP | A | 161 | 25.520 | −7.787 | 2.088 | 1.00 | 30.10 | 6 | C |
| ATOM | 1140 | C | TRP | A | 161 | 28.462 | −3.936 | 4.329 | 1.00 | 25.71 | 6 | C |
| ATOM | 1141 | O | TRP | A | 161 | 29.123 | −4.933 | 4.046 | 1.00 | 25.32 | 8 | O |
| ATOM | 1142 | N | PHE | A | 162 | 27.851 | −3.766 | 5.492 | 1.00 | 25.51 | 7 | N |
| ATOM | 1143 | CA | PHE | A | 162 | 27.813 | −4.820 | 6.492 | 1.00 | 25.62 | 6 | C |
| ATOM | 1144 | CB | PHE | A | 162 | 29.146 | −4.947 | 7.250 | 1.00 | 25.67 | 6 | C |
| ATOM | 1145 | CG | PHE | A | 162 | 29.507 | −3.752 | 8.100 | 1.00 | 25.29 | 6 | C |
| ATOM | 1146 | CD1 | PHE | A | 162 | 29.037 | −3.645 | 9.408 | 1.00 | 25.53 | 6 | C |
| ATOM | 1147 | CE1 | PHE | A | 162 | 29.370 | −2.568 | 10.197 | 1.00 | 24.54 | 6 | C |
| ATOM | 1148 | CZ | PHE | A | 162 | 30.196 | −1.566 | 9.693 | 1.00 | 25.18 | 6 | C |
| ATOM | 1149 | CE2 | PHE | A | 162 | 30.673 | −1.654 | 8.398 | 1.00 | 26.15 | 6 | C |
| ATOM | 1150 | CD2 | PHE | A | 162 | 30.336 | −2.758 | 7.610 | 1.00 | 26.22 | 6 | C |
| ATOM | 1151 | C | PHE | A | 162 | 26.622 | −4.541 | 7.401 | 1.00 | 26.20 | 6 | C |
| ATOM | 1152 | O | PHE | A | 162 | 26.070 | −3.432 | 7.386 | 1.00 | 25.37 | 8 | O |
| ATOM | 1153 | N | ARG | A | 163 | 26.202 | −5.541 | 8.166 | 1.00 | 26.36 | 7 | N |
| ATOM | 1154 | CA | ARG | A | 163 | 25.066 | −5.356 | 9.064 | 1.00 | 27.40 | 6 | C |
| ATOM | 1155 | CB | ARG | A | 163 | 23.899 | −6.261 | 8.644 | 1.00 | 28.06 | 6 | C |
| ATOM | 1156 | CG | ARG | A | 163 | 24.291 | −7.736 | 8.578 | 1.00 | 32.20 | 6 | C |
| ATOM | 1157 | CD | ARG | A | 163 | 23.123 | −8.716 | 8.483 | 1.00 | 36.60 | 6 | C |
| ATOM | 1158 | NE | ARG | A | 163 | 22.269 | −8.429 | 7.336 | 1.00 | 38.18 | 7 | N |
| ATOM | 1159 | CZ | ARG | A | 163 | 21.027 | −7.987 | 7.435 | 1.00 | 39.16 | 6 | C |
| ATOM | 1160 | NH1 | ARG | A | 163 | 20.488 | −7.791 | 8.633 | 1.00 | 40.03 | 7 | N |
| ATOM | 1161 | NH2 | ARG | A | 163 | 20.315 | −7.755 | 6.343 | 1.00 | 39.07 | 7 | N |
| ATOM | 1162 | C | ARG | A | 163 | 25.463 | −5.684 | 10.492 | 1.00 | 26.54 | 6 | C |
| ATOM | 1163 | O | ARG | A | 163 | 26.263 | −6.594 | 10.717 | 1.00 | 26.51 | 8 | O |
| ATOM | 1164 | N | THR | A | 164 | 24.947 | −4.906 | 11.442 | 1.00 | 26.05 | 7 | N |
| ATOM | 1165 | CA | THR | A | 164 | 25.115 | −5.214 | 12.867 | 1.00 | 26.45 | 6 | C |
| ATOM | 1166 | CB | THR | A | 164 | 25.641 | −4.012 | 13.660 | 1.00 | 26.07 | 6 | C |
| ATOM | 1167 | OG1 | THR | A | 164 | 24.684 | −2.945 | 13.600 | 1.00 | 25.53 | 8 | O |
| ATOM | 1168 | CG2 | THR | A | 164 | 26.901 | −3.434 | 13.016 | 1.00 | 26.06 | 6 | C |
| ATOM | 1169 | C | THR | A | 164 | 23.731 | −5.605 | 13.385 | 1.00 | 26.76 | 6 | C |
| ATOM | 1170 | O | THR | A | 164 | 22.777 | −5.676 | 12.613 | 1.00 | 26.66 | 8 | O |
| ATOM | 1171 | N | ARG | A | 165 | 23.602 | −5.828 | 14.684 | 1.00 | 27.66 | 7 | N |
| ATOM | 1172 | CA | ARG | A | 165 | 22.295 | −6.172 | 15.225 | 1.00 | 28.33 | 6 | C |
| ATOM | 1173 | CB | ARG | A | 165 | 22.429 | −6.707 | 16.661 | 1.00 | 28.64 | 6 | C |
| ATOM | 1174 | CG | ARG | A | 165 | 22.973 | −5.690 | 17.667 | 1.00 | 29.97 | 6 | C |
| ATOM | 1175 | CD | ARG | A | 165 | 23.038 | −6.213 | 19.116 | 1.00 | 32.01 | 6 | C |
| ATOM | 1176 | NE | ARG | A | 165 | 23.350 | −5.147 | 20.065 | 1.00 | 33.50 | 7 | N |
| ATOM | 1177 | CZ | ARG | A | 165 | 23.006 | −5.159 | 21.346 | 1.00 | 34.29 | 6 | C |
| ATOM | 1178 | NH1 | ARG | A | 165 | 22.335 | −6.191 | 21.846 | 1.00 | 35.50 | 7 | N |
| ATOM | 1179 | NH2 | ARG | A | 165 | 23.337 | −4.140 | 22.132 | 1.00 | 35.43 | 7 | N |
| ATOM | 1180 | C | ARG | A | 165 | 21.381 | −4.942 | 15.189 | 1.00 | 28.74 | 6 | C |
| ATOM | 1181 | O | ARG | A | 165 | 20.151 | −5.064 | 15.256 | 1.00 | 29.22 | 8 | O |
| ATOM | 1182 | N | SER | A | 166 | 21.982 | −3.759 | 15.061 | 1.00 | 27.82 | 7 | N |
| ATOM | 1183 | CA | SER | A | 166 | 21.228 | −2.508 | 15.132 | 1.00 | 27.68 | 6 | C |
| ATOM | 1184 | CB | SER | A | 166 | 21.906 | −1.526 | 16.103 | 1.00 | 27.82 | 6 | C |
| ATOM | 1185 | OG | SER | A | 166 | 22.192 | −2.112 | 17.363 | 1.00 | 30.66 | 8 | O |
| ATOM | 1186 | C | SER | A | 166 | 21.036 | −1.773 | 13.803 | 1.00 | 26.93 | 6 | C |
| ATOM | 1187 | O | SER | A | 166 | 20.139 | −0.936 | 13.688 | 1.00 | 26.34 | 8 | O |
| ATOM | 1188 | N | ALA | A | 167 | 21.871 | −2.066 | 12.811 | 1.00 | 25.82 | 7 | N |
| ATOM | 1189 | CA | ALA | A | 167 | 21.812 | −1.282 | 11.584 | 1.00 | 25.43 | 6 | C |
| ATOM | 1190 | CB | ALA | A | 167 | 22.478 | 0.087 | 11.839 | 1.00 | 24.92 | 6 | C |
| ATOM | 1191 | C | ALA | A | 167 | 22.485 | −1.926 | 10.390 | 1.00 | 24.77 | 6 | C |
| ATOM | 1192 | O | ALA | A | 167 | 23.257 | −2.884 | 10.538 | 1.00 | 24.32 | 8 | O |
| ATOM | 1193 | N | ILE | A | 168 | 22.197 | −1.371 | 9.210 | 1.00 | 24.07 | 7 | N |
| ATOM | 1194 | CA | ILE | A | 168 | 22.915 | −1.733 | 7.997 | 1.00 | 24.20 | 6 | C |
| ATOM | 1195 | CB | ILE | A | 168 | 21.979 | −2.205 | 6.840 | 1.00 | 24.66 | 6 | C |
| ATOM | 1196 | CG1 | ILE | A | 168 | 22.816 | −2.618 | 5.625 | 1.00 | 24.70 | 6 | C |
| ATOM | 1197 | CD1 | ILE | A | 168 | 22.053 | −3.476 | 4.600 | 1.00 | 26.32 | 6 | C |
| ATOM | 1198 | CG2 | ILE | A | 168 | 20.959 | −1.138 | 6.458 | 1.00 | 24.77 | 6 | C |
| ATOM | 1199 | C | ILE | A | 168 | 23.746 | −0.507 | 7.618 | 1.00 | 23.55 | 6 | C |
| ATOM | 1200 | O | ILE | A | 168 | 23.278 | 0.636 | 7.720 | 1.00 | 23.26 | 8 | O |
| ATOM | 1201 | N | ILE | A | 169 | 25.000 | −0.747 | 7.251 | 1.00 | 23.24 | 7 | N |
| ATOM | 1202 | CA | ILE | A | 169 | 25.950 | 0.326 | 6.933 | 1.00 | 22.80 | 6 | C |
| ATOM | 1203 | CB | ILE | A | 169 | 27.248 | 0.165 | 7.804 | 1.00 | 23.33 | 6 | C |
| ATOM | 1204 | CG1 | ILE | A | 169 | 26.972 | 0.486 | 9.276 | 1.00 | 22.62 | 6 | C |
| ATOM | 1205 | CD1 | ILE | A | 169 | 26.159 | −0.586 | 10.034 | 1.00 | 23.51 | 6 | C |
| ATOM | 1206 | CG2 | ILE | A | 169 | 28.389 | 1.042 | 7.284 | 1.00 | 22.78 | 6 | C |
| ATOM | 1207 | C | ILE | A | 169 | 26.269 | 0.178 | 5.461 | 1.00 | 22.89 | 6 | C |
| ATOM | 1208 | O | ILE | A | 169 | 26.661 | −0.898 | 5.037 | 1.00 | 22.95 | 8 | O |
| ATOM | 1209 | N | LEU | A | 170 | 26.081 | 1.253 | 4.694 | 1.00 | 22.59 | 7 | N |
| ATOM | 1210 | CA | LEU | A | 170 | 26.267 | 1.254 | 3.253 | 1.00 | 23.18 | 6 | C |
| ATOM | 1211 | CB | LEU | A | 170 | 24.942 | 1.584 | 2.565 | 1.00 | 23.34 | 6 | C |
| ATOM | 1212 | CG | LEU | A | 170 | 23.794 | 0.623 | 2.903 | 1.00 | 23.78 | 6 | C |
| ATOM | 1213 | CD1 | LEU | A | 170 | 22.445 | 1.335 | 2.810 | 1.00 | 24.60 | 6 | C |
| ATOM | 1214 | CD2 | LEU | A | 170 | 23.847 | −0.553 | 1.958 | 1.00 | 24.60 | 6 | C |
| ATOM | 1215 | C | LEU | A | 170 | 27.315 | 2.296 | 2.879 | 1.00 | 23.39 | 6 | C |
| ATOM | 1216 | O | LEU | A | 170 | 27.197 | 3.464 | 3.241 | 1.00 | 23.48 | 8 | O |
| ATOM | 1217 | N | HIS | A | 171 | 28.331 | 1.870 | 2.137 | 1.00 | 23.66 | 7 | N |
| ATOM | 1218 | CA | HIS | A | 171 | 29.408 | 2.766 | 1.751 | 1.00 | 24.20 | 6 | C |
| ATOM | 1219 | CB | HIS | A | 171 | 30.751 | 2.196 | 2.220 | 1.00 | 24.28 | 6 | C |
| ATOM | 1220 | CG | HIS | A | 171 | 31.930 | 3.061 | 1.885 | 1.00 | 26.53 | 6 | C |
| ATOM | 1221 | ND1 | HIS | A | 171 | 31.832 | 4.424 | 1.718 | 1.00 | 27.97 | 7 | N |
| ATOM | 1222 | CE1 | HIS | A | 171 | 33.029 | 4.922 | 1.446 | 1.00 | 29.66 | 6 | C |
| ATOM | 1223 | NE2 | HIS | A | 171 | 33.901 | 3.931 | 1.443 | 1.00 | 31.07 | 7 | N |
| ATOM | 1224 | CD2 | HIS | A | 171 | 33.239 | 2.755 | 1.712 | 1.00 | 27.93 | 6 | C |
| ATOM | 1225 | C | HIS | A | 171 | 29.408 | 2.911 | 0.238 | 1.00 | 24.50 | 6 | C |
| ATOM | 1226 | O | HIS | A | 171 | 29.715 | 1.950 | −0.481 | 1.00 | 24.62 | 8 | O |
| ATOM | 1227 | N | LEU | A | 172 | 29.065 | 4.107 | −0.229 | 1.00 | 24.25 | 7 | N |
| ATOM | 1228 | CA | LEU | A | 172 | 28.988 | 4.403 | −1.660 | 1.00 | 25.47 | 6 | C |
| ATOM | 1229 | CB | LEU | A | 172 | 27.897 | 5.462 | −1.915 | 1.00 | 25.00 | 6 | C |
| ATOM | 1230 | CG | LEU | A | 172 | 26.453 | 5.059 | −1.582 | 1.00 | 27.92 | 6 | C |
| ATOM | 1231 | CD1 | LEU | A | 172 | 25.466 | 6.107 | −2.079 | 1.00 | 28.86 | 6 | C |
| ATOM | 1232 | CD2 | LEU | A | 172 | 26.112 | 3.706 | −2.191 | 1.00 | 28.96 | 6 | C |
| ATOM | 1233 | C | LEU | A | 172 | 30.340 | 4.863 | −2.227 | 1.00 | 25.07 | 6 | C |
| ATOM | 1234 | O | LEU | A | 172 | 31.170 | 5.427 | −1.499 | 1.00 | 24.85 | 8 | O |
| ATOM | 1235 | N | SER | A | 173 | 30.537 | 4.664 | −3.528 | 1.00 | 25.35 | 7 | N |
| ATOM | 1236 | CA | SER | A | 173 | 31.809 | 5.003 | −4.182 | 1.00 | 25.98 | 6 | C |
| ATOM | 1237 | CB | SER | A | 173 | 31.879 | 4.396 | −5.590 | 1.00 | 26.00 | 6 | C |
| ATOM | 1238 | OG | SER | A | 173 | 30.838 | 4.902 | −6.402 | 1.00 | 24.57 | 8 | O |
| ATOM | 1239 | C | SER | A | 173 | 32.102 | 6.507 | −4.241 | 1.00 | 26.40 | 6 | C |
| ATOM | 1240 | O | SER | A | 173 | 33.217 | 6.912 | −4.578 | 1.00 | 27.27 | 8 | O |
| ATOM | 1241 | N | ASN | A | 174 | 31.106 | 7.336 | −3.938 | 1.00 | 26.37 | 7 | N |
| ATOM | 1242 | CA | ASN | A | 174 | 31.326 | 8.786 | −3.902 | 1.00 | 26.18 | 6 | C |
| ATOM | 1243 | CB | ASN | A | 174 | 30.071 | 9.556 | −4.355 | 1.00 | 26.32 | 6 | C |
| ATOM | 1244 | CG | ASN | A | 174 | 28.887 | 9.340 | −3.444 | 1.00 | 26.00 | 6 | C |
| ATOM | 1245 | OD1 | ASN | A | 174 | 28.971 | 8.617 | −2.442 | 1.00 | 24.32 | 8 | O |
| ATOM | 1246 | ND2 | ASN | A | 174 | 27.767 | 9.982 | −3.778 | 1.00 | 26.41 | 7 | N |
| ATOM | 1247 | C | ASN | A | 174 | 31.806 | 9.248 | −2.515 | 1.00 | 26.35 | 6 | C |
| ATOM | 1248 | O | ASN | A | 174 | 31.957 | 10.456 | −2.262 | 1.00 | 26.26 | 8 | O |
| ATOM | 1249 | N | GLY | A | 175 | 32.054 | 8.275 | −1.636 | 1.00 | 25.33 | 7 | N |
| ATOM | 1250 | CA | GLY | A | 175 | 32.550 | 8.526 | −0.291 | 1.00 | 25.96 | 6 | C |
| ATOM | 1251 | C | GLY | A | 175 | 31.475 | 8.588 | 0.787 | 1.00 | 25.46 | 6 | C |
| ATOM | 1252 | O | GLY | A | 175 | 31.776 | 8.570 | 1.980 | 1.00 | 25.69 | 8 | O |
| ATOM | 1253 | N | SER | A | 176 | 30.216 | 8.675 | 0.374 | 1.00 | 25.33 | 7 | N |
| ATOM | 1254 | CA | SER | A | 176 | 29.115 | 8.757 | 1.335 | 1.00 | 25.17 | 6 | C |
| ATOM | 1255 | CB | SER | A | 176 | 27.797 | 9.051 | 0.608 | 1.00 | 25.53 | 6 | C |
| ATOM | 1256 | OG | SER | A | 176 | 27.809 | 10.376 | 0.083 | 1.00 | 28.20 | 8 | O |
| ATOM | 1257 | C | SER | A | 176 | 28.961 | 7.466 | 2.134 | 1.00 | 24.26 | 6 | C |
| ATOM | 1258 | O | SER | A | 176 | 29.149 | 6.373 | 1.600 | 1.00 | 23.81 | 8 | O |
| ATOM | 1259 | N | VAL | A | 177 | 28.617 | 7.608 | 3.411 | 1.00 | 23.56 | 7 | N |
| ATOM | 1260 | CA | VAL | A | 177 | 28.325 | 6.464 | 4.267 | 1.00 | 23.31 | 6 | C |
| ATOM | 1261 | CB | VAL | A | 177 | 29.328 | 6.306 | 5.428 | 1.00 | 23.77 | 6 | C |
| ATOM | 1262 | CG1 | VAL | A | 177 | 28.852 | 5.217 | 6.392 | 1.00 | 24.27 | 6 | C |
| ATOM | 1263 | CG2 | VAL | A | 177 | 30.722 | 5.959 | 4.891 | 1.00 | 23.70 | 6 | C |
| ATOM | 1264 | C | VAL | A | 177 | 26.907 | 6.634 | 4.813 | 1.00 | 23.11 | 6 | C |
| ATOM | 1265 | O | VAL | A | 177 | 26.578 | 7.683 | 5.388 | 1.00 | 22.34 | 8 | O |
| ATOM | 1266 | N | GLN | A | 178 | 26.067 | 5.619 | 4.603 | 1.00 | 22.44 | 7 | N |
| ATOM | 1267 | CA | GLN | A | 178 | 24.684 | 5.670 | 5.074 | 1.00 | 22.30 | 6 | C |
| ATOM | 1268 | CB | GLN | A | 178 | 23.712 | 5.489 | 3.901 | 1.00 | 22.62 | 6 | C |
| ATOM | 1269 | CG | GLN | A | 178 | 22.230 | 5.506 | 4.290 | 1.00 | 23.11 | 6 | C |
| ATOM | 1270 | CD | GLN | A | 178 | 21.321 | 5.526 | 3.078 | 1.00 | 24.18 | 6 | C |
| ATOM | 1271 | OE1 | GLN | A | 178 | 21.713 | 5.999 | 2.004 | 1.00 | 24.06 | 8 | O |
| ATOM | 1272 | NE2 | GLN | A | 178 | 20.114 | 4.996 | 3.235 | 1.00 | 23.29 | 7 | N |
| ATOM | 1273 | C | GLN | A | 178 | 24.459 | 4.594 | 6.122 | 1.00 | 21.55 | 6 | C |
| ATOM | 1274 | O | GLN | A | 178 | 24.934 | 3.473 | 5.977 | 1.00 | 21.29 | 8 | O |
| ATOM | 1275 | N | ILE | A | 179 | 23.776 | 4.955 | 7.202 | 1.00 | 21.05 | 7 | N |
| ATOM | 1276 | CA | ILE | A | 179 | 23.473 | 3.995 | 8.264 | 1.00 | 21.59 | 6 | C |
| ATOM | 1277 | CB | ILE | A | 179 | 24.273 | 4.340 | 9.570 | 1.00 | 21.66 | 6 | C |
| ATOM | 1278 | CG1 | ILE | A | 179 | 25.773 | 4.433 | 9.280 | 1.00 | 21.63 | 6 | C |
| ATOM | 1279 | CD1 | ILE | A | 179 | 26.620 | 4.908 | 10.469 | 1.00 | 22.78 | 6 | C |
| ATOM | 1280 | CG2 | ILE | A | 179 | 24.021 | 3.268 | 10.644 | 1.00 | 22.47 | 6 | C |
| ATOM | 1281 | C | ILE | A | 179 | 21.976 | 4.045 | 8.532 | 1.00 | 21.86 | 6 | C |
| ATOM | 1282 | O | ILE | A | 179 | 21.451 | 5.108 | 8.902 | 1.00 | 21.55 | 8 | O |
| ATOM | 1283 | N | ASN | A | 180 | 21.304 | 2.910 | 8.335 | 1.00 | 22.26 | 7 | N |
| ATOM | 1284 | CA | ASN | A | 180 | 19.870 | 2.769 | 8.567 | 1.00 | 23.11 | 6 | C |
| ATOM | 1285 | CB | ASN | A | 180 | 19.183 | 2.006 | 7.422 | 1.00 | 22.91 | 6 | C |
| ATOM | 1286 | CG | ASN | A | 180 | 19.032 | 2.825 | 6.152 | 1.00 | 23.65 | 6 | C |
| ATOM | 1287 | OD1 | ASN | A | 180 | 19.694 | 3.852 | 5.955 | 1.00 | 23.20 | 8 | O |
| ATOM | 1288 | ND2 | ASN | A | 180 | 18.151 | 2.362 | 5.267 | 1.00 | 24.48 | 7 | N |
| ATOM | 1289 | C | ASN | A | 180 | 19.679 | 1.948 | 9.823 | 1.00 | 23.44 | 6 | C |
| ATOM | 1290 | O | ASN | A | 180 | 20.101 | 0.791 | 9.868 | 1.00 | 23.66 | 8 | O |
| ATOM | 1291 | N | PHE | A | 181 | 19.049 | 2.537 | 10.837 | 1.00 | 24.40 | 7 | N |
| ATOM | 1292 | CA | PHE | A | 181 | 18.801 | 1.842 | 12.097 | 1.00 | 25.35 | 6 | C |
| ATOM | 1293 | CB | PHE | A | 181 | 18.794 | 2.827 | 13.277 | 1.00 | 24.90 | 6 | C |
| ATOM | 1294 | CG | PHE | A | 181 | 20.128 | 3.511 | 13.500 | 1.00 | 25.75 | 6 | C |
| ATOM | 1295 | CD1 | PHE | A | 181 | 20.368 | 4.774 | 12.996 | 1.00 | 25.28 | 6 | C |
| ATOM | 1296 | CE1 | PHE | A | 181 | 21.604 | 5.392 | 13.186 | 1.00 | 26.59 | 6 | C |
| ATOM | 1297 | CZ | PHE | A | 181 | 22.597 | 4.744 | 13.886 | 1.00 | 26.44 | 6 | C |
| ATOM | 1298 | CE2 | PHE | A | 181 | 22.372 | 3.494 | 14.388 | 1.00 | 27.49 | 6 | C |
| ATOM | 1299 | CD2 | PHE | A | 181 | 21.139 | 2.873 | 14.190 | 1.00 | 25.89 | 6 | C |
| ATOM | 1300 | C | PHE | A | 181 | 17.498 | 1.049 | 12.007 | 1.00 | 26.42 | 6 | C |
| ATOM | 1301 | O | PHE | A | 181 | 16.460 | 1.585 | 11.601 | 1.00 | 26.61 | 8 | O |
| ATOM | 1302 | N | PHE | A | 182 | 17.564 | −0.229 | 12.369 | 1.00 | 27.90 | 7 | N |
| ATOM | 1303 | CA | PHE | A | 182 | 16.421 | −1.137 | 12.216 | 1.00 | 29.59 | 6 | C |
| ATOM | 1304 | CB | PHE | A | 182 | 16.851 | −2.602 | 12.398 | 1.00 | 29.28 | 6 | C |
| ATOM | 1305 | CG | PHE | A | 182 | 17.902 | −3.069 | 11.423 | 1.00 | 28.88 | 6 | C |
| ATOM | 1306 | CD1 | PHE | A | 182 | 19.009 | −3.762 | 11.875 | 1.00 | 28.98 | 6 | C |
| ATOM | 1307 | CE1 | PHE | A | 182 | 19.982 | −4.199 | 10.992 | 1.00 | 29.40 | 6 | C |
| ATOM | 1308 | CZ | PHE | A | 182 | 19.848 | −3.955 | 9.638 | 1.00 | 29.35 | 6 | C |
| ATOM | 1309 | CE2 | PHE | A | 182 | 18.743 | −3.263 | 9.172 | 1.00 | 29.15 | 6 | C |
| ATOM | 1310 | CD2 | PHE | A | 182 | 17.776 | −2.831 | 10.067 | 1.00 | 29.92 | 6 | C |
| ATOM | 1311 | C | PHE | A | 182 | 15.264 | −0.852 | 13.173 | 1.00 | 30.82 | 6 | C |
| ATOM | 1312 | O | PHE | A | 182 | 14.102 | −0.862 | 12.772 | 1.00 | 32.18 | 8 | O |
| ATOM | 1313 | N | GLN | A | 183 | 15.591 | −0.583 | 14.428 | 1.00 | 32.18 | 7 | N |
| ATOM | 1314 | CA | GLN | A | 183 | 14.591 | −0.443 | 15.491 | 1.00 | 33.29 | 6 | C |
| ATOM | 1315 | CB | GLN | A | 183 | 15.304 | −0.334 | 16.839 | 1.00 | 33.98 | 6 | C |
| ATOM | 1316 | CG | GLN | A | 183 | 14.415 | −0.518 | 18.052 | 1.00 | 37.89 | 6 | C |
| ATOM | 1317 | CD | GLN | A | 183 | 15.216 | −0.852 | 19.295 | 1.00 | 41.93 | 6 | C |
| ATOM | 1318 | OE1 | GLN | A | 183 | 16.179 | −0.149 | 19.629 | 1.00 | 44.34 | 8 | O |
| ATOM | 1319 | NE2 | GLN | A | 183 | 14.829 | −1.921 | 19.982 | 1.00 | 43.28 | 7 | N |
| ATOM | 1320 | C | GLN | A | 183 | 13.601 | 0.713 | 15.328 | 1.00 | 32.74 | 6 | C |
| ATOM | 1321 | O | GLN | A | 183 | 12.392 | 0.534 | 15.504 | 1.00 | 33.69 | 8 | O |
| ATOM | 1322 | N | ASP | A | 184 | 14.093 | 1.891 | 14.973 | 1.00 | 31.46 | 7 | N |
| ATOM | 1323 | CA | ASP | A | 184 | 13.210 | 3.048 | 14.889 | 1.00 | 30.53 | 6 | C |
| ATOM | 1324 | CB | ASP | A | 184 | 13.603 | 4.079 | 15.944 | 1.00 | 30.82 | 6 | C |
| ATOM | 1325 | CG | ASP | A | 184 | 15.022 | 4.596 | 15.763 | 1.00 | 31.38 | 6 | C |
| ATOM | 1326 | OD1 | ASP | A | 184 | 15.690 | 4.271 | 14.741 | 1.00 | 30.84 | 8 | O |
| ATOM | 1327 | OD2 | ASP | A | 184 | 15.547 | 5.350 | 16.601 | 1.00 | 31.85 | 8 | O |
| ATOM | 1328 | C | ASP | A | 184 | 13.142 | 3.688 | 13.510 | 1.00 | 29.56 | 6 | C |
| ATOM | 1329 | O | ASP | A | 184 | 12.552 | 4.752 | 13.340 | 1.00 | 28.90 | 8 | O |
| ATOM | 1330 | N | HIS | A | 185 | 13.761 | 3.036 | 12.527 | 1.00 | 28.47 | 7 | N |
| ATOM | 1331 | CA | HIS | A | 185 | 13.753 | 3.525 | 11.155 | 1.00 | 28.01 | 6 | C |
| ATOM | 1332 | CB | HIS | A | 185 | 12.316 | 3.630 | 10.643 | 1.00 | 28.20 | 6 | C |
| ATOM | 1333 | CG | HIS | A | 185 | 11.530 | 2.365 | 10.792 | 1.00 | 30.16 | 6 | C |
| ATOM | 1334 | ND1 | HIS | A | 185 | 11.815 | 1.223 | 10.075 | 1.00 | 30.90 | 7 | N |
| ATOM | 1335 | CE1 | HIS | A | 185 | 10.958 | 0.271 | 10.405 | 1.00 | 33.28 | 6 | C |
| ATOM | 1336 | NE2 | HIS | A | 185 | 10.126 | 0.756 | 11.310 | 1.00 | 32.23 | 7 | N |
| ATOM | 1337 | CD2 | HIS | A | 185 | 10.464 | 2.063 | 11.574 | 1.00 | 32.17 | 6 | C |
| ATOM | 1338 | C | HIS | A | 185 | 14.480 | 4.858 | 10.936 | 1.00 | 26.92 | 6 | C |
| ATOM | 1339 | O | HIS | A | 185 | 14.302 | 5.486 | 9.886 | 1.00 | 26.86 | 8 | O |
| ATOM | 1340 | N | THR | A | 186 | 15.279 | 5.303 | 11.905 | 1.00 | 26.53 | 7 | N |
| ATOM | 1341 | CA | THR | A | 186 | 16.040 | 6.552 | 11.714 | 1.00 | 25.66 | 6 | C |
| ATOM | 1342 | CB | THR | A | 186 | 16.544 | 7.190 | 13.041 | 1.00 | 25.96 | 6 | C |
| ATOM | 1343 | OG1 | THR | A | 186 | 17.269 | 6.228 | 13.806 | 1.00 | 25.64 | 8 | O |
| ATOM | 1344 | CG2 | THR | A | 186 | 15.389 | 7.622 | 13.962 | 1.00 | 26.85 | 6 | C |
| ATOM | 1345 | C | THR | A | 186 | 17.237 | 6.255 | 10.810 | 1.00 | 25.19 | 6 | C |
| ATOM | 1346 | O | THR | A | 186 | 17.713 | 5.121 | 10.769 | 1.00 | 24.35 | 8 | O |
| ATOM | 1347 | N | LYS | A | 187 | 17.753 | 7.278 | 10.133 | 1.00 | 24.27 | 7 | N |
| ATOM | 1348 | CA | LYS | A | 187 | 18.831 | 7.065 | 9.158 | 1.00 | 24.03 | 6 | C |
| ATOM | 1349 | CB | LYS | A | 187 | 18.240 | 6.886 | 7.748 | 1.00 | 24.02 | 6 | C |
| ATOM | 1350 | CG | LYS | A | 187 | 17.219 | 5.759 | 7.624 | 1.00 | 23.58 | 6 | C |
| ATOM | 1351 | CD | LYS | A | 187 | 16.588 | 5.693 | 6.221 | 1.00 | 25.16 | 6 | C |
| ATOM | 1352 | CE | LYS | A | 187 | 15.579 | 4.537 | 6.132 | 1.00 | 24.31 | 6 | C |
| ATOM | 1353 | NZ | LYS | A | 187 | 14.412 | 4.734 | 7.055 | 1.00 | 24.25 | 7 | N |
| ATOM | 1354 | C | LYS | A | 187 | 19.776 | 8.251 | 9.120 | 1.00 | 24.10 | 6 | C |
| ATOM | 1355 | O | LYS | A | 187 | 19.354 | 9.391 | 9.325 | 1.00 | 24.85 | 8 | O |
| ATOM | 1356 | N | LEU | A | 188 | 21.047 | 7.975 | 8.851 | 1.00 | 23.49 | 7 | N |
| ATOM | 1357 | CA | LEU | A | 188 | 22.055 | 9.014 | 8.674 | 1.00 | 23.57 | 6 | C |
| ATOM | 1358 | CB | LEU | A | 188 | 23.187 | 8.823 | 9.673 | 1.00 | 24.22 | 6 | C |
| ATOM | 1359 | CG | LEU | A | 188 | 22.913 | 8.997 | 11.160 | 1.00 | 25.94 | 6 | C |
| ATOM | 1360 | CD1 | LEU | A | 188 | 24.207 | 8.835 | 11.924 | 1.00 | 26.57 | 6 | C |
| ATOM | 1361 | CD2 | LEU | A | 188 | 22.320 | 10.366 | 11.419 | 1.00 | 28.31 | 6 | C |
| ATOM | 1362 | C | LEU | A | 188 | 22.677 | 8.861 | 7.297 | 1.00 | 23.47 | 6 | C |
| ATOM | 1363 | O | LEU | A | 188 | 22.961 | 7.735 | 6.877 | 1.00 | 22.53 | 8 | O |
| ATOM | 1364 | N | ILE | A | 189 | 22.901 | 9.978 | 6.605 | 1.00 | 23.57 | 7 | N |
| ATOM | 1365 | CA | ILE | A | 189 | 23.648 | 9.965 | 5.348 | 1.00 | 24.10 | 6 | C |
| ATOM | 1366 | CB | ILE | A | 189 | 22.783 | 10.443 | 4.181 | 1.00 | 24.59 | 6 | C |
| ATOM | 1367 | CG1 | ILE | A | 189 | 21.523 | 9.575 | 4.044 | 1.00 | 24.75 | 6 | C |
| ATOM | 1368 | CD1 | ILE | A | 189 | 20.426 | 10.284 | 3.221 | 1.00 | 25.98 | 6 | C |
| ATOM | 1369 | CG2 | ILE | A | 189 | 23.592 | 10.403 | 2.890 | 1.00 | 25.55 | 6 | C |
| ATOM | 1370 | C | ILE | A | 189 | 24.822 | 10.929 | 5.555 | 1.00 | 24.28 | 6 | C |
| ATOM | 1371 | O | ILE | A | 189 | 24.613 | 12.129 | 5.705 | 1.00 | 23.81 | 8 | O |
| ATOM | 1372 | N | LEU | A | 190 | 26.034 | 10.383 | 5.611 | 1.00 | 24.25 | 7 | N |
| ATOM | 1373 | CA | LEU | A | 190 | 27.240 | 11.151 | 5.873 | 1.00 | 25.06 | 6 | C |
| ATOM | 1374 | CB | LEU | A | 190 | 28.137 | 10.388 | 6.850 | 1.00 | 25.79 | 6 | C |
| ATOM | 1375 | CG | LEU | A | 190 | 27.580 | 10.239 | 8.263 | 1.00 | 27.20 | 6 | C |
| ATOM | 1376 | CD1 | LEU | A | 190 | 28.056 | 8.935 | 8.903 | 1.00 | 28.40 | 6 | C |
| ATOM | 1377 | CD2 | LEU | A | 190 | 28.010 | 11.468 | 9.085 | 1.00 | 30.40 | 6 | C |
| ATOM | 1378 | C | LEU | A | 190 | 28.020 | 11.363 | 4.595 | 1.00 | 25.17 | 6 | C |
| ATOM | 1379 | O | LEU | A | 190 | 28.271 | 10.409 | 3.869 | 1.00 | 24.80 | 8 | O |
| ATOM | 1380 | N | CYS | A | 191 | 28.422 | 12.608 | 4.345 | 1.00 | 25.78 | 7 | N |
| ATOM | 1381 | CA | CYS | A | 191 | 29.239 | 12.926 | 3.178 | 1.00 | 26.60 | 6 | C |
| ATOM | 1382 | CB | CYS | A | 191 | 28.477 | 13.798 | 2.184 | 1.00 | 26.74 | 6 | C |
| ATOM | 1383 | SG | CYS | A | 191 | 29.522 | 14.300 | 0.771 | 1.00 | 29.77 | 16 | S |
| ATOM | 1384 | C | CYS | A | 191 | 30.512 | 13.648 | 3.604 | 1.00 | 26.28 | 6 | C |
| ATOM | 1385 | O | CYS | A | 191 | 30.464 | 14.735 | 4.154 | 1.00 | 25.81 | 8 | O |
| ATOM | 1386 | N | PRO | A | 192 | 31.655 | 13.046 | 3.319 | 1.00 | 26.92 | 7 | N |
| ATOM | 1387 | CA | PRO | A | 192 | 32.937 | 13.622 | 3.713 | 1.00 | 27.35 | 6 | C |
| ATOM | 1388 | CB | PRO | A | 192 | 33.899 | 12.459 | 3.531 | 1.00 | 27.11 | 6 | C |
| ATOM | 1389 | CG | PRO | A | 192 | 33.290 | 11.633 | 2.441 | 1.00 | 27.39 | 6 | C |
| ATOM | 1390 | CD | PRO | A | 192 | 31.805 | 11.796 | 2.556 | 1.00 | 26.86 | 6 | C |
| ATOM | 1391 | C | PRO | A | 192 | 33.352 | 14.774 | 2.789 | 1.00 | 27.90 | 6 | C |
| ATOM | 1392 | O | PRO | A | 192 | 34.255 | 15.530 | 3.138 | 1.00 | 28.13 | 8 | O |
| ATOM | 1393 | N | LEU | A | 193 | 32.717 | 14.895 | 1.630 | 1.00 | 28.55 | 7 | N |
| ATOM | 1394 | CA | LEU | A | 193 | 33.070 | 15.966 | 0.689 | 1.00 | 29.46 | 6 | C |
| ATOM | 1395 | CB | LEU | A | 193 | 32.635 | 15.614 | −0.738 | 1.00 | 30.04 | 6 | C |
| ATOM | 1396 | CG | LEU | A | 193 | 33.225 | 14.307 | −1.290 | 1.00 | 30.91 | 6 | C |
| ATOM | 1397 | CD1 | LEU | A | 193 | 32.849 | 14.105 | −2.754 | 1.00 | 34.39 | 6 | C |
| ATOM | 1398 | CD2 | LEU | A | 193 | 34.738 | 14.306 | −1.131 | 1.00 | 33.74 | 6 | C |
| ATOM | 1399 | C | LEU | A | 193 | 32.420 | 17.257 | 1.163 | 1.00 | 29.81 | 6 | C |
| ATOM | 1400 | O | LEU | A | 193 | 33.048 | 18.321 | 1.164 | 1.00 | 30.32 | 8 | O |
| ATOM | 1401 | N | MET | A | 194 | 31.164 | 17.158 | 1.590 | 1.00 | 29.55 | 7 | N |
| ATOM | 1402 | CA | MET | A | 194 | 30.470 | 18.308 | 2.158 | 1.00 | 30.51 | 6 | C |
| ATOM | 1403 | CB | MET | A | 194 | 28.949 | 18.191 | 1.951 | 1.00 | 30.64 | 6 | C |
| ATOM | 1404 | CG | MET | A | 194 | 28.497 | 18.114 | 0.492 | 1.00 | 33.89 | 6 | C |
| ATOM | 1405 | SD | MET | A | 194 | 28.350 | 19.743 | −0.282 | 1.00 | 40.49 | 16 | S |
| ATOM | 1406 | CE | MET | A | 194 | 29.874 | 20.489 | 0.168 | 1.00 | 38.93 | 6 | C |
| ATOM | 1407 | C | MET | A | 194 | 30.772 | 18.452 | 3.657 | 1.00 | 29.73 | 6 | C |
| ATOM | 1408 | O | MET | A | 194 | 30.501 | 19.497 | 4.240 | 1.00 | 30.14 | 8 | O |
| ATOM | 1409 | N | ALA | A | 195 | 31.326 | 17.404 | 4.268 | 1.00 | 29.26 | 7 | N |
| ATOM | 1410 | CA | ALA | A | 195 | 31.543 | 17.367 | 5.722 | 1.00 | 28.29 | 6 | C |
| ATOM | 1411 | CB | ALA | A | 195 | 32.578 | 18.420 | 6.168 | 1.00 | 28.53 | 6 | C |
| ATOM | 1412 | C | ALA | A | 195 | 30.201 | 17.599 | 6.395 | 1.00 | 27.41 | 6 | C |
| ATOM | 1413 | O | ALA | A | 195 | 30.058 | 18.437 | 7.284 | 1.00 | 27.21 | 8 | O |
| ATOM | 1414 | N | ALA | A | 196 | 29.209 | 16.834 | 5.962 | 1.00 | 26.60 | 7 | N |
| ATOM | 1415 | CA | ALA | A | 196 | 27.846 | 17.048 | 6.403 | 1.00 | 25.81 | 6 | C |
| ATOM | 1416 | CB | ALA | A | 196 | 27.066 | 17.729 | 5.308 | 1.00 | 26.13 | 6 | C |
| ATOM | 1417 | C | ALA | A | 196 | 27.163 | 15.744 | 6.769 | 1.00 | 25.38 | 6 | C |
| ATOM | 1418 | O | ALA | A | 196 | 27.605 | 14.665 | 6.377 | 1.00 | 25.32 | 8 | O |
| ATOM | 1419 | N | VAL | A | 197 | 26.093 | 15.853 | 7.543 | 1.00 | 25.22 | 7 | N |
| ATOM | 1420 | CA | VAL | A | 197 | 25.289 | 14.691 | 7.888 | 1.00 | 24.87 | 6 | C |
| ATOM | 1421 | CB | VAL | A | 197 | 25.497 | 14.215 | 9.353 | 1.00 | 25.21 | 6 | C |
| ATOM | 1422 | CG1 | VAL | A | 197 | 25.102 | 15.298 | 10.359 | 1.00 | 24.80 | 6 | C |
| ATOM | 1423 | CG2 | VAL | A | 197 | 24.701 | 12.929 | 9.618 | 1.00 | 26.76 | 6 | C |
| ATOM | 1424 | C | VAL | A | 197 | 23.828 | 15.008 | 7.663 | 1.00 | 25.04 | 6 | C |
| ATOM | 1425 | O | VAL | A | 197 | 23.346 | 16.069 | 8.053 | 1.00 | 24.74 | 8 | O |
| ATOM | 1426 | N | THR | A | 198 | 23.120 | 14.077 | 7.027 | 1.00 | 24.63 | 7 | N |
| ATOM | 1427 | CA | THR | A | 198 | 21.687 | 14.218 | 6.852 | 1.00 | 25.28 | 6 | C |
| ATOM | 1428 | CB | THR | A | 198 | 21.309 | 13.955 | 5.385 | 1.00 | 25.27 | 6 | C |
| ATOM | 1429 | OG1 | THR | A | 198 | 21.801 | 15.031 | 4.591 | 1.00 | 24.40 | 8 | O |
| ATOM | 1430 | CG2 | THR | A | 198 | 19.779 | 14.020 | 5.178 | 1.00 | 25.08 | 6 | C |
| ATOM | 1431 | C | THR | A | 198 | 21.026 | 13.216 | 7.786 | 1.00 | 25.74 | 6 | C |
| ATOM | 1432 | O | THR | A | 198 | 21.331 | 12.032 | 7.743 | 1.00 | 25.91 | 8 | O |
| ATOM | 1433 | N | TYR | A | 199 | 20.161 | 13.709 | 8.666 | 1.00 | 26.25 | 7 | N |
| ATOM | 1434 | CA | TYR | A | 199 | 19.473 | 12.877 | 9.635 | 1.00 | 26.86 | 6 | C |
| ATOM | 1435 | CB | TYR | A | 199 | 19.636 | 13.490 | 11.034 | 1.00 | 26.82 | 6 | C |
| ATOM | 1436 | CG | TYR | A | 199 | 18.974 | 12.708 | 12.139 | 1.00 | 28.67 | 6 | C |
| ATOM | 1437 | CD1 | TYR | A | 199 | 19.057 | 11.328 | 12.182 | 1.00 | 29.47 | 6 | C |
| ATOM | 1438 | CE1 | TYR | A | 199 | 18.455 | 10.606 | 13.196 | 1.00 | 32.81 | 6 | C |
| ATOM | 1439 | CZ | TYR | A | 199 | 17.764 | 11.272 | 14.188 | 1.00 | 34.25 | 6 | C |
| ATOM | 1440 | OH | TYR | A | 199 | 17.163 | 10.561 | 15.205 | 1.00 | 38.37 | 8 | O |
| ATOM | 1441 | CE2 | TYR | A | 199 | 17.671 | 12.643 | 14.174 | 1.00 | 33.45 | 6 | C |
| ATOM | 1442 | CD2 | TYR | A | 199 | 18.277 | 13.357 | 13.151 | 1.00 | 30.73 | 6 | C |
| ATOM | 1443 | C | TYR | A | 199 | 18.000 | 12.780 | 9.268 | 1.00 | 26.79 | 6 | C |
| ATOM | 1444 | O | TYR | A | 199 | 17.345 | 13.803 | 9.042 | 1.00 | 26.93 | 8 | O |
| ATOM | 1445 | N | ILE | A | 200 | 17.502 | 11.550 | 9.168 | 1.00 | 26.74 | 7 | N |
| ATOM | 1446 | CA | ILE | A | 200 | 16.097 | 11.284 | 8.868 | 1.00 | 27.16 | 6 | C |
| ATOM | 1447 | CB | ILE | A | 200 | 15.948 | 10.266 | 7.714 | 1.00 | 26.97 | 6 | C |
| ATOM | 1448 | CG1 | ILE | A | 200 | 16.456 | 10.861 | 6.398 | 1.00 | 26.38 | 6 | C |
| ATOM | 1449 | CD1 | ILE | A | 200 | 16.462 | 9.862 | 5.225 | 1.00 | 26.08 | 6 | C |
| ATOM | 1450 | CG2 | ILE | A | 200 | 14.482 | 9.858 | 7.550 | 1.00 | 26.89 | 6 | C |
| ATOM | 1451 | C | ILE | A | 200 | 15.545 | 10.696 | 10.157 | 1.00 | 27.75 | 6 | C |
| ATOM | 1452 | O | ILE | A | 200 | 15.995 | 9.639 | 10.595 | 1.00 | 27.06 | 8 | O |
| ATOM | 1453 | N | ASP | A | 201 | 14.605 | 11.393 | 10.790 | 1.00 | 29.03 | 7 | N |
| ATOM | 1454 | CA | ASP | A | 201 | 14.122 | 10.955 | 12.099 | 1.00 | 30.64 | 6 | C |
| ATOM | 1455 | CB | ASP | A | 201 | 13.854 | 12.161 | 13.017 | 1.00 | 31.21 | 6 | C |
| ATOM | 1456 | CG | ASP | A | 201 | 12.624 | 12.960 | 12.611 | 1.00 | 32.40 | 6 | C |
| ATOM | 1457 | OD1 | ASP | A | 201 | 11.793 | 12.475 | 11.812 | 1.00 | 33.54 | 8 | O |
| ATOM | 1458 | OD2 | ASP | A | 201 | 12.397 | 14.096 | 13.071 | 1.00 | 35.66 | 8 | O |
| ATOM | 1459 | C | ASP | A | 201 | 12.921 | 10.002 | 12.011 | 1.00 | 31.64 | 6 | C |
| ATOM | 1460 | O | ASP | A | 201 | 12.446 | 9.696 | 10.916 | 1.00 | 31.11 | 8 | O |
| ATOM | 1461 | N | GLU | A | 202 | 12.443 | 9.525 | 13.160 | 1.00 | 33.23 | 7 | N |
| ATOM | 1462 | CA | GLU | A | 202 | 11.361 | 8.542 | 13.173 | 1.00 | 34.74 | 6 | C |
| ATOM | 1463 | CB | GLU | A | 202 | 11.138 | 7.940 | 14.571 | 1.00 | 35.27 | 6 | C |
| ATOM | 1464 | CG | GLU | A | 202 | 11.226 | 8.923 | 15.722 | 1.00 | 38.23 | 6 | C |
| ATOM | 1465 | CD | GLU | A | 202 | 12.657 | 9.148 | 16.179 | 1.00 | 41.73 | 6 | C |
| ATOM | 1466 | OE1 | GLU | A | 202 | 13.209 | 8.265 | 16.888 | 1.00 | 43.97 | 8 | O |
| ATOM | 1467 | OE2 | GLU | A | 202 | 13.230 | 10.204 | 15.827 | 1.00 | 41.84 | 8 | O |
| ATOM | 1468 | C | GLU | A | 202 | 10.050 | 9.052 | 12.583 | 1.00 | 35.39 | 6 | C |
| ATOM | 1469 | O | GLU | A | 202 | 9.156 | 8.265 | 12.282 | 1.00 | 35.88 | 8 | O |
| ATOM | 1470 | N | LYS | A | 203 | 9.938 | 10.360 | 12.398 | 1.00 | 35.90 | 7 | N |
| ATOM | 1471 | CA | LYS | A | 203 | 8.740 | 10.919 | 11.790 | 1.00 | 36.58 | 6 | C |
| ATOM | 1472 | CB | LYS | A | 203 | 8.337 | 12.212 | 12.508 | 1.00 | 37.33 | 6 | C |
| ATOM | 1473 | CG | LYS | A | 203 | 8.233 | 12.040 | 14.025 | 1.00 | 38.95 | 6 | C |
| ATOM | 1474 | CD | LYS | A | 203 | 7.774 | 13.318 | 14.718 | 1.00 | 42.84 | 6 | C |
| ATOM | 1475 | CE | LYS | A | 203 | 7.529 | 13.084 | 16.207 | 1.00 | 44.43 | 6 | C |
| ATOM | 1476 | NZ | LYS | A | 203 | 6.740 | 14.186 | 16.831 | 1.00 | 46.60 | 7 | N |
| ATOM | 1477 | C | LYS | A | 203 | 8.957 | 11.146 | 10.295 | 1.00 | 36.53 | 6 | C |
| ATOM | 1478 | O | LYS | A | 203 | 8.072 | 11.626 | 9.594 | 1.00 | 36.51 | 8 | O |
| ATOM | 1479 | N | ARG | A | 204 | 10.139 | 10.765 | 9.814 | 1.00 | 36.40 | 7 | N |
| ATOM | 1480 | CA | ARG | A | 204 | 10.523 | 10.908 | 8.405 | 1.00 | 36.41 | 6 | C |
| ATOM | 1481 | CB | ARG | A | 204 | 9.467 | 10.330 | 7.468 | 1.00 | 36.80 | 6 | C |
| ATOM | 1482 | CG | ARG | A | 204 | 9.260 | 8.842 | 7.667 | 1.00 | 38.81 | 6 | C |
| ATOM | 1483 | CD | ARG | A | 204 | 8.316 | 8.209 | 6.664 | 1.00 | 42.51 | 6 | C |
| ATOM | 1484 | NE | ARG | A | 204 | 7.496 | 7.175 | 7.291 | 1.00 | 47.42 | 7 | N |
| ATOM | 1485 | CZ | ARG | A | 204 | 7.602 | 5.882 | 7.028 | 1.00 | 48.72 | 6 | C |
| ATOM | 1486 | NH1 | ARG | A | 204 | 8.500 | 5.465 | 6.151 | 1.00 | 50.90 | 7 | N |
| ATOM | 1487 | NH2 | ARG | A | 204 | 6.819 | 5.003 | 7.640 | 1.00 | 49.75 | 7 | N |
| ATOM | 1488 | C | ARG | A | 204 | 10.872 | 12.344 | 8.053 | 1.00 | 36.27 | 6 | C |
| ATOM | 1489 | O | ARG | A | 204 | 11.058 | 12.702 | 6.886 | 1.00 | 35.30 | 8 | O |
| ATOM | 1490 | N | ASP | A | 205 | 10.958 | 13.163 | 9.091 | 1.00 | 36.42 | 7 | N |
| ATOM | 1491 | CA | ASP | A | 205 | 11.386 | 14.534 | 8.939 | 1.00 | 37.02 | 6 | C |
| ATOM | 1492 | CB | ASP | A | 205 | 11.021 | 15.342 | 10.176 | 1.00 | 37.71 | 6 | C |
| ATOM | 1493 | CG | ASP | A | 205 | 10.499 | 16.710 | 9.831 | 1.00 | 40.93 | 6 | C |
| ATOM | 1494 | OD1 | ASP | A | 205 | 11.285 | 17.681 | 9.909 | 1.00 | 42.87 | 8 | O |
| ATOM | 1495 | OD2 | ASP | A | 205 | 9.316 | 16.901 | 9.460 | 1.00 | 44.32 | 8 | O |
| ATOM | 1496 | C | ASP | A | 205 | 12.895 | 14.482 | 8.761 | 1.00 | 36.46 | 6 | C |
| ATOM | 1497 | O | ASP | A | 205 | 13.564 | 13.562 | 9.247 | 1.00 | 36.31 | 8 | O |
| ATOM | 1498 | N | PHE | A | 206 | 13.446 | 15.458 | 8.060 | 1.00 | 35.73 | 7 | N |
| ATOM | 1499 | CA | PHE | A | 206 | 14.866 | 15.414 | 7.794 | 1.00 | 34.97 | 6 | C |
| ATOM | 1500 | CB | PHE | A | 206 | 15.096 | 14.828 | 6.403 | 1.00 | 35.12 | 6 | C |
| ATOM | 1501 | CG | PHE | A | 206 | 14.542 | 15.677 | 5.298 | 1.00 | 35.80 | 6 | C |
| ATOM | 1502 | CD1 | PHE | A | 206 | 15.337 | 16.622 | 4.663 | 1.00 | 36.66 | 6 | C |
| ATOM | 1503 | CE1 | PHE | A | 206 | 14.831 | 17.407 | 3.647 | 1.00 | 36.61 | 6 | C |
| ATOM | 1504 | CZ | PHE | A | 206 | 13.516 | 17.260 | 3.255 | 1.00 | 37.26 | 6 | C |
| ATOM | 1505 | CE2 | PHE | A | 206 | 12.711 | 16.328 | 3.881 | 1.00 | 37.55 | 6 | C |
| ATOM | 1506 | CD2 | PHE | A | 206 | 13.222 | 15.546 | 4.899 | 1.00 | 37.21 | 6 | C |
| ATOM | 1507 | C | PHE | A | 206 | 15.514 | 16.787 | 7.870 | 1.00 | 34.23 | 6 | C |
| ATOM | 1508 | O | PHE | A | 206 | 14.846 | 17.813 | 7.736 | 1.00 | 34.04 | 8 | O |
| ATOM | 1509 | N | ARG | A | 207 | 16.829 | 16.775 | 8.059 | 1.00 | 32.42 | 7 | N |
| ATOM | 1510 | CA | ARG | A | 207 | 17.639 | 17.973 | 8.080 | 1.00 | 31.15 | 6 | C |
| ATOM | 1511 | CB | ARG | A | 207 | 17.721 | 18.530 | 9.504 | 1.00 | 31.65 | 6 | C |
| ATOM | 1512 | CG | ARG | A | 207 | 17.048 | 19.867 | 9.750 | 1.00 | 34.21 | 6 | C |
| ATOM | 1513 | CD | ARG | A | 207 | 15.763 | 20.088 | 9.013 | 1.00 | 37.48 | 6 | C |
| ATOM | 1514 | NE | ARG | A | 207 | 14.911 | 21.073 | 9.676 | 1.00 | 39.43 | 7 | N |
| ATOM | 1515 | CZ | ARG | A | 207 | 13.596 | 20.943 | 9.761 | 1.00 | 40.61 | 6 | C |
| ATOM | 1516 | NH1 | ARG | A | 207 | 13.009 | 19.882 | 9.221 | 1.00 | 40.43 | 7 | N |
| ATOM | 1517 | NH2 | ARG | A | 207 | 12.865 | 21.865 | 10.375 | 1.00 | 41.27 | 7 | N |
| ATOM | 1518 | C | ARG | A | 207 | 19.044 | 17.589 | 7.646 | 1.00 | 29.46 | 6 | C |
| ATOM | 1519 | O | ARG | A | 207 | 19.516 | 16.488 | 7.939 | 1.00 | 28.39 | 8 | O |
| ATOM | 1520 | N | THR | A | 208 | 19.717 | 18.509 | 6.973 | 1.00 | 27.70 | 7 | N |
| ATOM | 1521 | CA | THR | A | 208 | 21.117 | 18.324 | 6.635 | 1.00 | 26.90 | 6 | C |
| ATOM | 1522 | CB | THR | A | 208 | 21.340 | 18.595 | 5.155 | 1.00 | 27.18 | 6 | C |
| ATOM | 1523 | OG1 | THR | A | 208 | 20.704 | 17.563 | 4.396 | 1.00 | 28.05 | 8 | O |
| ATOM | 1524 | CG2 | THR | A | 208 | 22.824 | 18.467 | 4.810 | 1.00 | 26.41 | 6 | C |
| ATOM | 1525 | C | THR | A | 208 | 21.914 | 19.317 | 7.477 | 1.00 | 26.37 | 6 | C |
| ATOM | 1526 | O | THR | A | 208 | 21.589 | 20.491 | 7.498 | 1.00 | 26.33 | 8 | O |
| ATOM | 1527 | N | TYR | A | 209 | 22.938 | 18.837 | 8.179 | 1.00 | 25.78 | 7 | N |
| ATOM | 1528 | CA | TYR | A | 209 | 23.731 | 19.689 | 9.074 | 1.00 | 25.54 | 6 | C |
| ATOM | 1529 | CB | TYR | A | 209 | 23.648 | 19.141 | 10.505 | 1.00 | 25.31 | 6 | C |
| ATOM | 1530 | CG | TYR | A | 209 | 22.274 | 19.111 | 11.095 | 1.00 | 25.56 | 6 | C |
| ATOM | 1531 | CD1 | TYR | A | 209 | 21.540 | 17.936 | 11.127 | 1.00 | 27.19 | 6 | C |
| ATOM | 1532 | CE1 | TYR | A | 209 | 20.278 | 17.897 | 11.678 | 1.00 | 27.25 | 6 | C |
| ATOM | 1533 | CZ | TYR | A | 209 | 19.731 | 19.048 | 12.198 | 1.00 | 27.58 | 6 | C |
| ATOM | 1534 | OH | TYR | A | 209 | 18.471 | 19.004 | 12.741 | 1.00 | 28.08 | 8 | O |
| ATOM | 1535 | CE2 | TYR | A | 209 | 20.439 | 20.235 | 12.180 | 1.00 | 27.33 | 6 | C |
| ATOM | 1536 | CD2 | TYR | A | 209 | 21.705 | 20.259 | 11.632 | 1.00 | 27.03 | 6 | C |
| ATOM | 1537 | C | TYR | A | 209 | 25.199 | 19.673 | 8.713 | 1.00 | 25.06 | 6 | C |
| ATOM | 1538 | O | TYR | A | 209 | 25.746 | 18.617 | 8.387 | 1.00 | 24.78 | 8 | O |
| ATOM | 1539 | N | ARG | A | 210 | 25.856 | 20.828 | 8.799 | 1.00 | 24.92 | 7 | N |
| ATOM | 1540 | CA | ARG | A | 210 | 27.298 | 20.864 | 8.640 | 1.00 | 24.96 | 6 | C |
| ATOM | 1541 | CB | ARG | A | 210 | 27.773 | 22.300 | 8.432 | 1.00 | 25.52 | 6 | C |
| ATOM | 1542 | CG | ARG | A | 210 | 28.709 | 22.478 | 7.288 | 1.00 | 29.28 | 6 | C |
| ATOM | 1543 | CD | ARG | A | 210 | 28.950 | 23.949 | 6.924 | 1.00 | 31.30 | 6 | C |
| ATOM | 1544 | NE | ARG | A | 210 | 28.560 | 24.209 | 5.547 | 1.00 | 36.97 | 7 | N |
| ATOM | 1545 | CZ | ARG | A | 210 | 29.356 | 24.017 | 4.512 | 1.00 | 37.48 | 6 | C |
| ATOM | 1546 | NH1 | ARG | A | 210 | 30.595 | 23.578 | 4.709 | 1.00 | 38.89 | 7 | N |
| ATOM | 1547 | NH2 | ARG | A | 210 | 28.925 | 24.276 | 3.294 | 1.00 | 37.04 | 7 | N |
| ATOM | 1548 | C | ARG | A | 210 | 27.877 | 20.350 | 9.951 | 1.00 | 24.74 | 6 | C |
| ATOM | 1549 | O | ARG | A | 210 | 27.537 | 20.862 | 11.030 | 1.00 | 24.12 | 8 | O |
| ATOM | 1550 | N | LEU | A | 211 | 28.762 | 19.362 | 9.867 | 1.00 | 23.95 | 7 | N |
| ATOM | 1551 | CA | LEU | A | 211 | 29.324 | 18.756 | 11.076 | 1.00 | 24.48 | 6 | C |
| ATOM | 1552 | CB | LEU | A | 211 | 30.228 | 17.573 | 10.714 | 1.00 | 24.81 | 6 | C |
| ATOM | 1553 | CG | LEU | A | 211 | 29.472 | 16.344 | 10.216 | 1.00 | 25.82 | 6 | C |
| ATOM | 1554 | CD1 | LEU | A | 211 | 30.420 | 15.356 | 9.535 | 1.00 | 28.04 | 6 | C |
| ATOM | 1555 | CD2 | LEU | A | 211 | 28.746 | 15.673 | 11.405 | 1.00 | 27.62 | 6 | C |
| ATOM | 1556 | C | LEU | A | 211 | 30.085 | 19.742 | 11.950 | 1.00 | 24.79 | 6 | C |
| ATOM | 1557 | O | LEU | A | 211 | 29.979 | 19.701 | 13.183 | 1.00 | 24.62 | 8 | O |
| ATOM | 1558 | N | SER | A | 212 | 30.859 | 20.623 | 11.323 | 1.00 | 24.24 | 7 | N |
| ATOM | 1559 | CA | SER | A | 212 | 31.621 | 21.621 | 12.080 | 1.00 | 24.90 | 6 | C |
| ATOM | 1560 | CB | SER | A | 212 | 32.659 | 22.353 | 11.195 | 1.00 | 24.80 | 6 | C |
| ATOM | 1561 | OG | SER | A | 212 | 32.048 | 23.064 | 10.141 | 1.00 | 25.93 | 8 | O |
| ATOM | 1562 | C | SER | A | 212 | 30.704 | 22.602 | 12.816 | 1.00 | 24.67 | 6 | C |
| ATOM | 1563 | O | SER | A | 212 | 31.068 | 23.094 | 13.880 | 1.00 | 24.77 | 8 | O |
| ATOM | 1564 | N | LEU | A | 213 | 29.507 | 22.855 | 12.286 | 1.00 | 24.57 | 7 | N |
| ATOM | 1565 | CA | LEU | A | 213 | 28.556 | 23.740 | 12.974 | 1.00 | 24.86 | 6 | C |
| ATOM | 1566 | CB | LEU | A | 213 | 27.526 | 24.316 | 12.002 | 1.00 | 24.39 | 6 | C |
| ATOM | 1567 | CG | LEU | A | 213 | 28.111 | 25.341 | 11.026 | 1.00 | 24.18 | 6 | C |
| ATOM | 1568 | CD1 | LEU | A | 213 | 27.047 | 25.832 | 10.040 | 1.00 | 23.86 | 6 | C |
| ATOM | 1569 | CD2 | LEU | A | 213 | 28.745 | 26.527 | 11.790 | 1.00 | 24.15 | 6 | C |
| ATOM | 1570 | C | LEU | A | 213 | 27.855 | 23.053 | 14.151 | 1.00 | 25.20 | 6 | C |
| ATOM | 1571 | O | LEU | A | 213 | 27.463 | 23.714 | 15.123 | 1.00 | 24.93 | 8 | O |
| ATOM | 1572 | N | LEU | A | 214 | 27.672 | 21.737 | 14.059 | 1.00 | 25.82 | 7 | N |
| ATOM | 1573 | CA | LEU | A | 214 | 27.120 | 20.975 | 15.197 | 1.00 | 26.69 | 6 | C |
| ATOM | 1574 | CB | LEU | A | 214 | 26.877 | 19.513 | 14.812 | 1.00 | 26.57 | 6 | C |
| ATOM | 1575 | CG | LEU | A | 214 | 25.718 | 19.265 | 13.847 | 1.00 | 26.82 | 6 | C |
| ATOM | 1576 | CD1 | LEU | A | 214 | 25.672 | 17.794 | 13.400 | 1.00 | 26.32 | 6 | C |
| ATOM | 1577 | CD2 | LEU | A | 214 | 24.401 | 19.674 | 14.490 | 1.00 | 26.19 | 6 | C |
| ATOM | 1578 | C | LEU | A | 214 | 28.132 | 21.043 | 16.335 | 1.00 | 27.38 | 6 | C |
| ATOM | 1579 | O | LEU | A | 214 | 27.778 | 21.077 | 17.525 | 1.00 | 27.59 | 8 | O |
| ATOM | 1580 | N | GLU | A | 215 | 29.405 | 21.044 | 15.961 | 1.00 | 27.94 | 7 | N |
| ATOM | 1581 | CA | GLU | A | 215 | 30.480 | 21.129 | 16.938 | 1.00 | 29.14 | 6 | C |
| ATOM | 1582 | CB | GLU | A | 215 | 31.832 | 20.999 | 16.233 | 1.00 | 29.52 | 6 | C |
| ATOM | 1583 | CG | GLU | A | 215 | 33.024 | 21.144 | 17.151 | 1.00 | 31.92 | 6 | C |
| ATOM | 1584 | CD | GLU | A | 215 | 34.330 | 20.820 | 16.457 | 1.00 | 35.42 | 6 | C |
| ATOM | 1585 | OE1 | GLU | A | 215 | 34.317 | 20.077 | 15.442 | 1.00 | 37.83 | 8 | O |
| ATOM | 1586 | OE2 | GLU | A | 215 | 35.371 | 21.311 | 16.932 | 1.00 | 38.01 | 8 | O |
| ATOM | 1587 | C | GLU | A | 215 | 30.398 | 22.459 | 17.681 | 1.00 | 29.04 | 6 | C |
| ATOM | 1588 | O | GLU | A | 215 | 30.588 | 22.537 | 18.901 | 1.00 | 28.72 | 8 | O |
| ATOM | 1589 | N | GLU | A | 216 | 30.074 | 23.512 | 16.939 | 1.00 | 28.90 | 7 | N |
| ATOM | 1590 | CA | GLU | A | 216 | 30.031 | 24.846 | 17.518 | 1.00 | 28.96 | 6 | C |
| ATOM | 1591 | CB | GLU | A | 216 | 30.291 | 25.894 | 16.435 | 1.00 | 29.27 | 6 | C |
| ATOM | 1592 | CG | GLU | A | 216 | 31.704 | 25.831 | 15.898 | 1.00 | 32.49 | 6 | C |
| ATOM | 1593 | CD | GLU | A | 216 | 31.881 | 26.639 | 14.634 | 1.00 | 35.60 | 6 | C |
| ATOM | 1594 | OE1 | GLU | A | 216 | 31.369 | 27.788 | 14.599 | 1.00 | 31.06 | 8 | O |
| ATOM | 1595 | OE2 | GLU | A | 216 | 32.525 | 26.100 | 13.693 | 1.00 | 36.77 | 8 | O |
| ATOM | 1596 | C | GLU | A | 216 | 28.728 | 25.158 | 18.229 | 1.00 | 28.41 | 6 | C |
| ATOM | 1597 | O | GLU | A | 216 | 28.737 | 25.791 | 19.282 | 1.00 | 27.84 | 8 | O |
| ATOM | 1598 | N | TYR | A | 217 | 27.611 | 24.686 | 17.675 | 1.00 | 28.11 | 7 | N |
| ATOM | 1599 | CA | TYR | A | 217 | 26.295 | 25.039 | 18.201 | 1.00 | 28.19 | 6 | C |
| ATOM | 1600 | CB | TYR | A | 217 | 25.402 | 25.548 | 17.070 | 1.00 | 28.42 | 6 | C |
| ATOM | 1601 | CG | TYR | A | 217 | 25.858 | 26.892 | 16.546 | 1.00 | 29.01 | 6 | C |
| ATOM | 1602 | CD1 | TYR | A | 217 | 26.601 | 26.996 | 15.375 | 1.00 | 30.89 | 6 | C |
| ATOM | 1603 | CE1 | TYR | A | 217 | 27.031 | 28.241 | 14.908 | 1.00 | 31.06 | 6 | C |
| ATOM | 1604 | CZ | TYR | A | 217 | 26.711 | 29.381 | 15.631 | 1.00 | 31.94 | 6 | C |
| ATOM | 1605 | OH | TYR | A | 217 | 27.125 | 30.617 | 15.199 | 1.00 | 32.46 | 8 | O |
| ATOM | 1606 | CE2 | TYR | A | 217 | 25.980 | 29.288 | 16.794 | 1.00 | 30.74 | 6 | C |
| ATOM | 1607 | CD2 | TYR | A | 217 | 25.557 | 28.057 | 17.240 | 1.00 | 30.04 | 6 | C |
| ATOM | 1608 | C | TYR | A | 217 | 25.578 | 23.951 | 19.007 | 1.00 | 28.54 | 6 | C |
| ATOM | 1609 | O | TYR | A | 217 | 24.570 | 24.239 | 19.662 | 1.00 | 28.35 | 8 | O |
| ATOM | 1610 | N | GLY | A | 218 | 26.092 | 22.723 | 18.947 | 1.00 | 28.00 | 7 | N |
| ATOM | 1611 | CA | GLY | A | 218 | 25.524 | 21.602 | 19.696 | 1.00 | 28.61 | 6 | C |
| ATOM | 1612 | C | GLY | A | 218 | 24.399 | 20.886 | 18.972 | 1.00 | 28.81 | 6 | C |
| ATOM | 1613 | O | GLY | A | 218 | 23.959 | 21.325 | 17.914 | 1.00 | 28.46 | 8 | O |
| ATOM | 1614 | N | CYS | A | 219 | 23.937 | 19.770 | 19.535 | 1.00 | 29.39 | 7 | N |
| ATOM | 1615 | CA | CYS | A | 219 | 22.804 | 19.047 | 18.974 | 1.00 | 30.37 | 6 | C |
| ATOM | 1616 | CB | CYS | A | 219 | 23.192 | 18.175 | 17.771 | 1.00 | 30.56 | 6 | C |
| ATOM | 1617 | SG | CYS | A | 219 | 24.248 | 16.767 | 18.144 | 1.00 | 32.81 | 16 | S |
| ATOM | 1618 | C | CYS | A | 219 | 22.139 | 18.210 | 20.045 | 1.00 | 30.63 | 6 | C |
| ATOM | 1619 | O | CYS | A | 219 | 22.665 | 18.061 | 21.146 | 1.00 | 30.45 | 8 | O |
| ATOM | 1620 | N | CYS | A | 220 | 20.978 | 17.661 | 19.719 | 1.00 | 31.60 | 7 | N |
| ATOM | 1621 | CA | CYS | A | 220 | 20.224 | 16.889 | 20.695 | 1.00 | 32.42 | 6 | C |
| ATOM | 1622 | CB | CYS | A | 220 | 18.800 | 16.657 | 20.189 | 1.00 | 32.94 | 6 | C |
| ATOM | 1623 | SG | CYS | A | 220 | 18.712 | 15.547 | 18.750 | 1.00 | 35.67 | 16 | S |
| ATOM | 1624 | C | CYS | A | 220 | 20.877 | 15.543 | 20.957 | 1.00 | 32.66 | 6 | C |
| ATOM | 1625 | O | CYS | A | 220 | 21.647 | 15.042 | 20.142 | 1.00 | 31.36 | 8 | O |
| ATOM | 1626 | N | LYS | A | 221 | 20.593 | 14.975 | 22.123 | 1.00 | 33.15 | 7 | N |
| ATOM | 1627 | CA | LYS | A | 221 | 20.997 | 13.620 | 22.378 | 1.00 | 34.52 | 6 | C |
| ATOM | 1628 | CB | LYS | A | 221 | 20.503 | 13.168 | 23.762 | 1.00 | 35.08 | 6 | C |
| ATOM | 1629 | CG | LYS | A | 221 | 20.396 | 11.664 | 23.931 | 1.00 | 37.45 | 6 | C |
| ATOM | 1630 | CD | LYS | A | 221 | 19.537 | 11.281 | 25.137 | 1.00 | 41.50 | 6 | C |
| ATOM | 1631 | CE | LYS | A | 221 | 19.220 | 9.793 | 25.114 | 1.00 | 43.16 | 6 | C |
| ATOM | 1632 | NZ | LYS | A | 221 | 18.022 | 9.437 | 25.937 | 1.00 | 44.32 | 7 | N |
| ATOM | 1633 | C | LYS | A | 221 | 20.200 | 12.966 | 21.261 | 1.00 | 34.63 | 6 | C |
| ATOM | 1634 | O | LYS | A | 221 | 19.268 | 13.553 | 20.750 | 1.00 | 36.08 | 8 | O |
| ATOM | 1635 | N | GLU | A | 222 | 20.543 | 11.777 | 20.837 | 1.00 | 34.66 | 7 | N |
| ATOM | 1636 | CA | GLU | A | 222 | 19.779 | 11.175 | 19.742 | 1.00 | 33.55 | 6 | C |
| ATOM | 1637 | CB | GLU | A | 222 | 18.390 | 11.802 | 19.530 | 1.00 | 34.71 | 6 | C |
| ATOM | 1638 | CG | GLU | A | 222 | 17.261 | 11.284 | 20.433 | 1.00 | 37.70 | 6 | C |
| ATOM | 1639 | CD | GLU | A | 222 | 16.841 | 12.297 | 21.487 | 1.00 | 41.31 | 6 | C |
| ATOM | 1640 | OE1 | GLU | A | 222 | 17.183 | 13.496 | 21.338 | 1.00 | 40.54 | 8 | O |
| ATOM | 1641 | OE2 | GLU | A | 222 | 16.163 | 11.901 | 22.471 | 1.00 | 44.12 | 8 | O |
| ATOM | 1642 | C | GLU | A | 222 | 20.601 | 11.365 | 18.494 | 1.00 | 31.77 | 6 | C |
| ATOM | 1643 | O | GLU | A | 222 | 21.102 | 10.399 | 17.961 | 1.00 | 31.65 | 8 | O |
| ATOM | 1644 | N | LEU | A | 223 | 20.737 | 12.600 | 18.011 | 1.00 | 30.02 | 7 | N |
| ATOM | 1645 | CA | LEU | A | 223 | 21.627 | 12.797 | 16.869 | 1.00 | 28.45 | 6 | C |
| ATOM | 1646 | CB | LEU | A | 223 | 21.482 | 14.186 | 16.236 | 1.00 | 28.53 | 6 | C |
| ATOM | 1647 | CG | LEU | A | 223 | 22.461 | 14.498 | 15.100 | 1.00 | 28.64 | 6 | C |
| ATOM | 1648 | CD1 | LEU | A | 223 | 22.416 | 13.409 | 14.023 | 1.00 | 28.96 | 6 | C |
| ATOM | 1649 | CD2 | LEU | A | 223 | 22.160 | 15.859 | 14.506 | 1.00 | 28.18 | 6 | C |
| ATOM | 1650 | C | LEU | A | 223 | 23.044 | 12.546 | 17.374 | 1.00 | 27.23 | 6 | C |
| ATOM | 1651 | O | LEU | A | 223 | 23.826 | 11.848 | 16.738 | 1.00 | 25.93 | 8 | O |
| ATOM | 1652 | N | ALA | A | 224 | 23.356 | 13.073 | 18.559 | 1.00 | 26.68 | 7 | N |
| ATOM | 1653 | CA | ALA | A | 224 | 24.683 | 12.874 | 19.127 | 1.00 | 26.35 | 6 | C |
| ATOM | 1654 | CB | ALA | A | 224 | 24.822 | 13.605 | 20.474 | 1.00 | 26.82 | 6 | C |
| ATOM | 1655 | C | ALA | A | 224 | 25.033 | 11.405 | 19.292 | 1.00 | 25.93 | 6 | C |
| ATOM | 1656 | O | ALA | A | 224 | 26.161 | 10.988 | 19.001 | 1.00 | 24.68 | 8 | O |
| ATOM | 1657 | N | SER | A | 225 | 24.076 | 10.618 | 19.772 | 1.00 | 25.86 | 7 | N |
| ATOM | 1658 | CA | SER | A | 225 | 24.341 | 9.209 | 20.004 | 1.00 | 26.40 | 6 | C |
| ATOM | 1659 | CB | SER | A | 225 | 23.220 | 8.566 | 20.831 | 1.00 | 26.51 | 6 | C |
| ATOM | 1660 | OG | SER | A | 225 | 21.999 | 8.630 | 20.130 | 1.00 | 31.54 | 8 | O |
| ATOM | 1661 | C | SER | A | 225 | 24.520 | 8.478 | 18.670 | 1.00 | 25.53 | 6 | C |
| ATOM | 1662 | O | SER | A | 225 | 25.331 | 7.550 | 18.559 | 1.00 | 25.79 | 8 | O |
| ATOM | 1663 | N | ARG | A | 226 | 23.759 | 8.893 | 17.668 | 1.00 | 24.77 | 7 | N |
| ATOM | 1664 | CA | ARG | A | 226 | 23.867 | 8.272 | 16.354 | 1.00 | 24.66 | 6 | C |
| ATOM | 1665 | CB | ARG | A | 226 | 22.660 | 8.614 | 15.485 | 1.00 | 24.86 | 6 | C |
| ATOM | 1666 | CG | ARG | A | 226 | 21.403 | 7.808 | 15.900 | 1.00 | 25.80 | 6 | C |
| ATOM | 1667 | CD | ARG | A | 226 | 20.076 | 8.383 | 15.422 | 1.00 | 27.28 | 6 | C |
| ATOM | 1668 | NE | ARG | A | 226 | 18.934 | 7.584 | 15.889 | 1.00 | 27.33 | 7 | N |
| ATOM | 1669 | CZ | ARG | A | 226 | 18.443 | 7.622 | 17.129 | 1.00 | 29.35 | 6 | C |
| ATOM | 1670 | NH1 | ARG | A | 226 | 18.974 | 8.429 | 18.041 | 1.00 | 28.48 | 7 | N |
| ATOM | 1671 | NH2 | ARG | A | 226 | 17.403 | 6.864 | 17.458 | 1.00 | 30.48 | 7 | N |
| ATOM | 1672 | C | ARG | A | 226 | 25.202 | 8.615 | 15.686 | 1.00 | 24.36 | 6 | C |
| ATOM | 1673 | O | ARG | A | 226 | 25.783 | 7.777 | 14.985 | 1.00 | 24.15 | 8 | O |
| ATOM | 1674 | N | LEU | A | 227 | 25.687 | 9.835 | 15.915 | 1.00 | 24.00 | 7 | N |
| ATOM | 1675 | CA | LEU | A | 227 | 26.993 | 10.243 | 15.385 | 1.00 | 24.49 | 6 | C |
| ATOM | 1676 | CB | LEU | A | 227 | 27.197 | 11.751 | 15.522 | 1.00 | 25.07 | 6 | C |
| ATOM | 1677 | CG | LEU | A | 227 | 26.409 | 12.631 | 14.546 | 1.00 | 25.52 | 6 | C |
| ATOM | 1678 | CD1 | LEU | A | 227 | 26.575 | 14.116 | 14.901 | 1.00 | 25.92 | 6 | C |
| ATOM | 1679 | CD2 | LEU | A | 227 | 26.832 | 12.394 | 13.087 | 1.00 | 27.53 | 6 | C |
| ATOM | 1680 | C | LEU | A | 227 | 28.139 | 9.470 | 16.066 | 1.00 | 24.12 | 6 | C |
| ATOM | 1681 | O | LEU | A | 227 | 29.154 | 9.164 | 15.436 | 1.00 | 23.19 | 8 | O |
| ATOM | 1682 | N | ARG | A | 228 | 27.981 | 9.151 | 17.351 | 1.00 | 23.81 | 7 | N |
| ATOM | 1683 | CA | ARG | A | 228 | 28.973 | 8.310 | 18.026 | 1.00 | 24.03 | 6 | C |
| ATOM | 1684 | CB | ARG | A | 228 | 28.654 | 8.178 | 19.526 | 1.00 | 23.73 | 6 | C |
| ATOM | 1685 | CG | ARG | A | 228 | 29.133 | 9.349 | 20.409 | 1.00 | 26.03 | 6 | C |
| ATOM | 1686 | CD | ARG | A | 228 | 28.765 | 9.173 | 21.909 | 1.00 | 31.16 | 6 | C |
| ATOM | 1687 | NE | ARG | A | 228 | 27.857 | 10.250 | 22.283 | 1.00 | 36.96 | 7 | N |
| ATOM | 1688 | CZ | ARG | A | 228 | 26.632 | 10.098 | 22.737 | 1.00 | 36.30 | 6 | C |
| ATOM | 1689 | NH1 | ARG | A | 228 | 26.118 | 8.889 | 22.949 | 1.00 | 37.17 | 7 | N |
| ATOM | 1690 | NH2 | ARG | A | 228 | 25.928 | 11.174 | 23.012 | 1.00 | 38.46 | 7 | N |
| ATOM | 1691 | C | ARG | A | 228 | 28.992 | 6.929 | 17.375 | 1.00 | 23.69 | 6 | C |
| ATOM | 1692 | O | ARG | A | 228 | 30.053 | 6.366 | 17.098 | 1.00 | 23.53 | 8 | O |
| ATOM | 1693 | N | TYR | A | 229 | 27.805 | 6.371 | 17.157 | 1.00 | 24.48 | 7 | N |
| ATOM | 1694 | CA | TYR | A | 229 | 27.673 | 5.069 | 16.509 | 1.00 | 24.19 | 6 | C |
| ATOM | 1695 | CB | TYR | A | 229 | 26.196 | 4.703 | 16.373 | 1.00 | 24.66 | 6 | C |
| ATOM | 1696 | CG | TYR | A | 229 | 25.971 | 3.265 | 15.968 | 1.00 | 25.51 | 6 | C |
| ATOM | 1697 | CD1 | TYR | A | 229 | 25.931 | 2.264 | 16.930 | 1.00 | 26.27 | 6 | C |
| ATOM | 1698 | CE1 | TYR | A | 229 | 25.730 | 0.944 | 16.580 | 1.00 | 28.27 | 6 | C |
| ATOM | 1699 | CZ | TYR | A | 229 | 25.563 | 0.598 | 15.258 | 1.00 | 26.61 | 6 | C |
| ATOM | 1700 | OH | TYR | A | 229 | 25.367 | −0.732 | 14.947 | 1.00 | 27.01 | 8 | O |
| ATOM | 1701 | CE2 | TYR | A | 229 | 25.601 | 1.561 | 14.274 | 1.00 | 27.00 | 6 | C |
| ATOM | 1702 | CD2 | TYR | A | 229 | 25.807 | 2.902 | 14.628 | 1.00 | 25.01 | 6 | C |
| ATOM | 1703 | C | TYR | A | 229 | 28.312 | 5.117 | 15.110 | 1.00 | 23.94 | 6 | C |
| ATOM | 1704 | O | TYR | A | 229 | 29.032 | 4.211 | 14.713 | 1.00 | 23.19 | 8 | O |
| ATOM | 1705 | N | ALA | A | 230 | 28.044 | 6.190 | 14.376 | 1.00 | 23.63 | 7 | N |
| ATOM | 1706 | CA | ALA | A | 230 | 28.627 | 6.350 | 13.033 | 1.00 | 23.80 | 6 | C |
| ATOM | 1707 | CB | ALA | A | 230 | 28.128 | 7.634 | 12.391 | 1.00 | 23.61 | 6 | C |
| ATOM | 1708 | C | ALA | A | 230 | 30.165 | 6.297 | 13.042 | 1.00 | 23.70 | 6 | C |
| ATOM | 1709 | O | ALA | A | 230 | 30.790 | 5.651 | 12.186 | 1.00 | 23.89 | 8 | O |
| ATOM | 1710 | N | ARG | A | 231 | 30.780 | 6.956 | 14.016 | 1.00 | 23.74 | 7 | N |
| ATOM | 1711 | CA | ARG | A | 231 | 32.234 | 6.917 | 14.138 | 1.00 | 24.06 | 6 | C |
| ATOM | 1712 | CB | ARG | A | 231 | 32.710 | 7.811 | 15.283 | 1.00 | 24.34 | 6 | C |
| ATOM | 1713 | CG | ARG | A | 231 | 34.223 | 7.959 | 15.371 | 1.00 | 26.14 | 6 | C |
| ATOM | 1714 | CD | ARG | A | 231 | 34.902 | 7.010 | 16.354 | 1.00 | 28.93 | 6 | C |
| ATOM | 1715 | NE | ARG | A | 231 | 36.355 | 7.205 | 16.370 | 1.00 | 31.60 | 7 | N |
| ATOM | 1716 | CZ | ARG | A | 231 | 36.961 | 8.234 | 16.942 | 1.00 | 33.08 | 6 | C |
| ATOM | 1717 | NH1 | ARG | A | 231 | 36.251 | 9.163 | 17.571 | 1.00 | 34.81 | 7 | N |
| ATOM | 1718 | NH2 | ARG | A | 231 | 38.283 | 8.339 | 16.895 | 1.00 | 35.10 | 7 | N |
| ATOM | 1719 | C | ARG | A | 231 | 32.755 | 5.487 | 14.313 | 1.00 | 23.52 | 6 | C |
| ATOM | 1720 | O | ARG | A | 231 | 33.743 | 5.103 | 13.686 | 1.00 | 24.27 | 8 | O |
| ATOM | 1721 | N | THR | A | 232 | 32.084 | 4.707 | 15.155 | 1.00 | 23.50 | 7 | N |
| ATOM | 1722 | CA | THR | A | 232 | 32.442 | 3.312 | 15.370 | 1.00 | 23.31 | 6 | C |
| ATOM | 1723 | CB | THR | A | 232 | 31.491 | 2.700 | 16.424 | 1.00 | 24.05 | 6 | C |
| ATOM | 1724 | OG1 | THR | A | 232 | 31.636 | 3.415 | 17.666 | 1.00 | 24.01 | 8 | O |
| ATOM | 1725 | CG2 | THR | A | 232 | 31.905 | 1.260 | 16.755 | 1.00 | 24.30 | 6 | C |
| ATOM | 1726 | C | THR | A | 232 | 32.330 | 2.533 | 14.056 | 1.00 | 23.53 | 6 | C |
| ATOM | 1727 | O | THR | A | 232 | 33.188 | 1.705 | 13.718 | 1.00 | 22.83 | 8 | O |
| ATOM | 1728 | N | MET | A | 233 | 31.258 | 2.792 | 13.317 | 1.00 | 23.02 | 7 | N |
| ATOM | 1729 | CA | MET | A | 233 | 31.071 | 2.114 | 12.042 | 1.00 | 23.97 | 6 | C |
| ATOM | 1730 | CB | MET | A | 233 | 29.681 | 2.389 | 11.474 | 1.00 | 23.70 | 6 | C |
| ATOM | 1731 | CG | MET | A | 233 | 28.548 | 1.893 | 12.339 | 1.00 | 24.89 | 6 | C |
| ATOM | 1732 | SD | MET | A | 233 | 28.603 | 0.124 | 12.651 | 1.00 | 25.92 | 16 | S |
| ATOM | 1733 | CE | MET | A | 233 | 29.192 | 0.099 | 14.342 | 1.00 | 27.03 | 6 | C |
| ATOM | 1734 | C | MET | A | 233 | 32.141 | 2.509 | 11.032 | 1.00 | 23.90 | 6 | C |
| ATOM | 1735 | O | MET | A | 233 | 32.602 | 1.671 | 10.255 | 1.00 | 24.29 | 8 | O |
| ATOM | 1736 | N | VAL | A | 234 | 32.528 | 3.779 | 11.034 | 1.00 | 24.08 | 7 | N |
| ATOM | 1737 | CA | VAL | A | 234 | 33.561 | 4.232 | 10.099 | 1.00 | 25.69 | 6 | C |
| ATOM | 1738 | CB | VAL | A | 234 | 33.647 | 5.767 | 10.036 | 1.00 | 25.75 | 6 | C |
| ATOM | 1739 | CG1 | VAL | A | 234 | 34.896 | 6.230 | 9.251 | 1.00 | 26.58 | 6 | C |
| ATOM | 1740 | CG2 | VAL | A | 234 | 32.379 | 6.325 | 9.415 | 1.00 | 26.02 | 6 | C |
| ATOM | 1741 | C | VAL | A | 234 | 34.912 | 3.586 | 10.434 | 1.00 | 26.22 | 6 | C |
| ATOM | 1742 | O | VAL | A | 234 | 35.628 | 3.137 | 9.531 | 1.00 | 27.03 | 8 | O |
| ATOM | 1743 | N | ASP | A | 235 | 35.242 | 3.502 | 11.723 | 1.00 | 27.15 | 7 | N |
| ATOM | 1744 | CA | ASP | A | 235 | 36.470 | 2.813 | 12.153 | 1.00 | 27.94 | 6 | C |
| ATOM | 1745 | CB | ASP | A | 235 | 36.618 | 2.810 | 13.685 | 1.00 | 28.58 | 6 | C |
| ATOM | 1746 | CG | ASP | A | 235 | 37.272 | 4.061 | 14.226 | 1.00 | 30.71 | 6 | C |
| ATOM | 1747 | OD1 | ASP | A | 235 | 38.060 | 4.718 | 13.498 | 1.00 | 31.20 | 8 | O |
| ATOM | 1748 | OD2 | ASP | A | 235 | 37.073 | 4.460 | 15.397 | 1.00 | 33.27 | 8 | O |
| ATOM | 1749 | C | ASP | A | 235 | 36.458 | 1.372 | 11.655 | 1.00 | 27.90 | 6 | C |
| ATOM | 1750 | O | ASP | A | 235 | 37.482 | 0.848 | 11.198 | 1.00 | 27.86 | 8 | O |
| ATOM | 1751 | N | LYS | A | 236 | 35.301 | 0.718 | 11.758 | 1.00 | 28.17 | 7 | N |
| ATOM | 1752 | CA | LYS | A | 236 | 35.167 | −0.661 | 11.292 | 1.00 | 29.21 | 6 | C |
| ATOM | 1753 | CB | LYS | A | 236 | 33.805 | −1.255 | 11.651 | 1.00 | 28.80 | 6 | C |
| ATOM | 1754 | CG | LYS | A | 236 | 33.766 | −2.755 | 11.453 | 1.00 | 31.44 | 6 | C |
| ATOM | 1755 | CD | LYS | A | 236 | 32.463 | −3.365 | 11.880 | 1.00 | 33.65 | 6 | C |
| ATOM | 1756 | CE | LYS | A | 236 | 32.677 | −4.762 | 12.424 | 1.00 | 35.73 | 6 | C |
| ATOM | 1757 | NZ | LYS | A | 236 | 33.676 | −5.582 | 11.682 | 1.00 | 33.42 | 7 | N |
| ATOM | 1758 | C | LYS | A | 236 | 35.412 | −0.779 | 9.781 | 1.00 | 29.51 | 6 | C |
| ATOM | 1759 | O | LYS | A | 236 | 36.136 | −1.676 | 9.336 | 1.00 | 29.82 | 8 | O |
| ATOM | 1760 | N | LEU | A | 237 | 34.813 | 0.123 | 9.006 | 1.00 | 29.66 | 7 | N |
| ATOM | 1761 | CA | LEU | A | 237 | 35.000 | 0.144 | 7.556 | 1.00 | 30.62 | 6 | C |
| ATOM | 1762 | CB | LEU | A | 237 | 34.222 | 1.310 | 6.923 | 1.00 | 29.80 | 6 | C |
| ATOM | 1763 | CG | LEU | A | 237 | 32.701 | 1.153 | 6.793 | 1.00 | 28.82 | 6 | C |
| ATOM | 1764 | CD1 | LEU | A | 237 | 32.026 | 2.447 | 6.386 | 1.00 | 29.45 | 6 | C |
| ATOM | 1765 | CD2 | LEU | A | 237 | 32.362 | 0.031 | 5.797 | 1.00 | 28.42 | 6 | C |
| ATOM | 1766 | C | LEU | A | 237 | 36.488 | 0.282 | 7.234 | 1.00 | 31.81 | 6 | C |
| ATOM | 1767 | O | LEU | A | 237 | 37.002 | −0.373 | 6.326 | 1.00 | 31.85 | 8 | O |
| ATOM | 1768 | N | LEU | A | 238 | 37.174 | 1.133 | 7.986 | 1.00 | 33.83 | 7 | N |
| ATOM | 1769 | CA | LEU | A | 238 | 38.610 | 1.323 | 7.794 | 1.00 | 36.16 | 6 | C |
| ATOM | 1770 | CB | LEU | A | 238 | 39.098 | 2.564 | 8.539 | 1.00 | 35.64 | 6 | C |
| ATOM | 1771 | CG | LEU | A | 238 | 38.733 | 3.870 | 7.834 | 1.00 | 35.77 | 6 | C |
| ATOM | 1772 | CD1 | LEU | A | 238 | 38.772 | 5.047 | 8.792 | 1.00 | 35.86 | 6 | C |
| ATOM | 1773 | CD2 | LEU | A | 238 | 39.644 | 4.104 | 6.622 | 1.00 | 35.95 | 6 | C |
| ATOM | 1774 | C | LEU | A | 238 | 39.427 | 0.110 | 8.219 | 1.00 | 37.98 | 6 | C |
| ATOM | 1775 | O | LEU | A | 238 | 40.483 | −0.154 | 7.650 | 1.00 | 38.49 | 8 | O |
| ATOM | 1776 | N | SER | A | 239 | 38.939 | −0.627 | 9.210 | 1.00 | 40.20 | 7 | N |
| ATOM | 1777 | CA | SER | A | 239 | 39.658 | −1.793 | 9.715 | 1.00 | 42.61 | 6 | C |
| ATOM | 1778 | CB | SER | A | 239 | 39.069 | −2.256 | 11.048 | 1.00 | 42.53 | 6 | C |
| ATOM | 1779 | OG | SER | A | 239 | 37.938 | −3.081 | 10.828 | 1.00 | 41.85 | 8 | O |
| ATOM | 1780 | C | SER | A | 239 | 39.597 | −2.942 | 8.723 | 1.00 | 44.55 | 6 | C |
| ATOM | 1781 | O | SER | A | 239 | 40.564 | −3.680 | 8.551 | 1.00 | 45.33 | 8 | O |
| ATOM | 1782 | N | SER | A | 240 | 38.448 | −3.099 | 8.076 | 1.00 | 46.75 | 7 | N |
| ATOM | 1783 | CA | SER | A | 240 | 38.266 | −4.176 | 7.118 | 1.00 | 48.84 | 6 | C |
| ATOM | 1784 | CB | SER | A | 240 | 36.812 | −4.656 | 7.104 | 1.00 | 48.94 | 6 | C |
| ATOM | 1785 | OG | SER | A | 240 | 35.913 | −3.574 | 6.949 | 1.00 | 50.43 | 8 | O |
| ATOM | 1786 | C | SER | A | 240 | 38.705 | −3.724 | 5.734 | 1.00 | 49.93 | 6 | C |
| ATOM | 1787 | O | SER | A | 240 | 38.692 | −4.508 | 4.790 | 1.00 | 50.59 | 8 | O |
| ATOM | 1788 | N | ALA | A | 241 | 39.105 | −2.458 | 5.635 | 1.00 | 51.23 | 7 | N |
| ATOM | 1789 | CA | ALA | A | 241 | 39.580 | −1.867 | 4.382 | 1.00 | 52.19 | 6 | C |
| ATOM | 1790 | CB | ALA | A | 241 | 40.848 | −1.074 | 4.625 | 1.00 | 52.24 | 6 | C |
| ATOM | 1791 | C | ALA | A | 241 | 39.819 | −2.906 | 3.294 | 1.00 | 52.79 | 6 | C |
| ATOM | 1792 | O | ALA | A | 241 | 40.907 | −3.488 | 3.238 | 1.00 | 53.19 | 8 | O |
| ATOM | 1793 | OXT | ALA | A | 241 | 38.934 | −3.162 | 2.470 | 1.00 | 53.32 | 8 | O |
| ATOM | 1794 | N | ALA | B | 20 | −18.462 | 10.374 | −32.692 | 1.00 | 40.15 | 7 | N |
| ATOM | 1795 | CA | ALA | B | 20 | −18.787 | 10.792 | −31.295 | 1.00 | 39.44 | 6 | C |
| ATOM | 1796 | CB | ALA | B | 20 | −19.597 | 12.064 | −31.300 | 1.00 | 39.55 | 6 | C |
| ATOM | 1797 | C | ALA | B | 20 | −19.538 | 9.676 | −30.576 | 1.00 | 38.88 | 6 | C |
| ATOM | 1798 | O | ALA | B | 20 | −20.122 | 8.802 | −31.212 | 1.00 | 38.93 | 8 | O |
| ATOM | 1799 | N | LEU | B | 21 | −19.515 | 9.710 | −29.249 | 1.00 | 38.33 | 7 | N |
| ATOM | 1800 | CA | LEU | B | 21 | −20.162 | 8.679 | −28.452 | 1.00 | 37.95 | 6 | C |
| ATOM | 1801 | CB | LEU | B | 21 | −19.852 | 8.865 | −26.969 | 1.00 | 38.46 | 6 | C |
| ATOM | 1802 | CG | LEU | B | 21 | −18.459 | 8.484 | −26.486 | 1.00 | 39.45 | 6 | C |
| ATOM | 1803 | CD1 | LEU | B | 21 | −18.389 | 8.609 | −24.970 | 1.00 | 40.66 | 6 | C |
| ATOM | 1804 | CD2 | LEU | B | 21 | −18.114 | 7.069 | −26.923 | 1.00 | 40.86 | 6 | C |
| ATOM | 1805 | C | LEU | B | 21 | −21.663 | 8.693 | −28.674 | 1.00 | 37.29 | 6 | C |
| ATOM | 1806 | O | LEU | B | 21 | −22.288 | 7.643 | −28.802 | 1.00 | 36.75 | 8 | O |
| ATOM | 1807 | N | SER | B | 22 | −22.235 | 9.894 | −28.706 | 1.00 | 36.31 | 7 | N |
| ATOM | 1808 | CA | SER | B | 22 | −23.662 | 10.057 | −28.948 | 1.00 | 35.97 | 6 | C |
| ATOM | 1809 | CB | SER | B | 22 | −24.027 | 11.551 | −28.960 | 1.00 | 36.01 | 6 | C |
| ATOM | 1810 | OG | SER | B | 22 | −25.386 | 11.740 | −29.301 | 1.00 | 38.45 | 8 | O |
| ATOM | 1811 | C | SER | B | 22 | −24.081 | 9.355 | −30.248 | 1.00 | 34.55 | 6 | C |
| ATOM | 1812 | O | SER | B | 22 | −25.046 | 8.597 | −30.264 | 1.00 | 34.41 | 8 | O |
| ATOM | 1813 | N | ASP | B | 23 | −23.346 | 9.583 | −31.332 | 1.00 | 33.54 | 7 | N |
| ATOM | 1814 | CA | ASP | B | 23 | −23.635 | 8.906 | −32.596 | 1.00 | 32.88 | 6 | C |
| ATOM | 1815 | CB | ASP | B | 23 | −22.679 | 9.371 | −33.696 | 1.00 | 32.93 | 6 | C |
| ATOM | 1816 | CG | ASP | B | 23 | −22.915 | 10.820 | −34.120 | 1.00 | 34.64 | 6 | C |
| ATOM | 1817 | OD1 | ASP | B | 23 | −24.013 | 11.362 | −33.878 | 1.00 | 34.23 | 8 | O |
| ATOM | 1818 | OD2 | ASP | B | 23 | −22.050 | 11.485 | −34.718 | 1.00 | 35.74 | 8 | O |
| ATOM | 1819 | C | ASP | B | 23 | −23.534 | 7.375 | −32.453 | 1.00 | 32.47 | 6 | C |
| ATOM | 1820 | O | ASP | B | 23 | −24.386 | 6.633 | −32.945 | 1.00 | 31.81 | 8 | O |
| ATOM | 1821 | N | MET | B | 24 | −22.490 | 6.906 | −31.781 | 1.00 | 31.81 | 7 | N |
| ATOM | 1822 | CA | MET | B | 24 | −22.307 | 5.459 | −31.625 | 1.00 | 32.01 | 6 | C |
| ATOM | 1823 | CB | MET | B | 24 | −20.997 | 5.143 | −30.894 | 1.00 | 32.02 | 6 | C |
| ATOM | 1824 | CG | MET | B | 24 | −20.641 | 3.656 | −30.899 | 1.00 | 33.91 | 6 | C |
| ATOM | 1825 | SD | MET | B | 24 | −19.040 | 3.300 | −30.170 | 1.00 | 36.01 | 16 | S |
| ATOM | 1826 | CE | MET | B | 24 | −17.947 | 3.957 | −31.436 | 1.00 | 36.64 | 6 | C |
| ATOM | 1827 | C | MET | B | 24 | −23.492 | 4.849 | −30.882 | 1.00 | 31.30 | 6 | C |
| ATOM | 1828 | O | MET | B | 24 | −23.984 | 3.784 | −31.245 | 1.00 | 31.16 | 8 | O |
| ATOM | 1829 | N | LEU | B | 25 | −23.956 | 5.539 | −29.846 | 1.00 | 31.37 | 7 | N |
| ATOM | 1830 | CA | LEU | B | 25 | −25.086 | 5.057 | −29.060 | 1.00 | 31.48 | 6 | C |
| ATOM | 1831 | CB | LEU | B | 25 | −25.356 | 5.979 | −27.871 | 1.00 | 31.48 | 6 | C |
| ATOM | 1832 | CG | LEU | B | 25 | −26.522 | 5.539 | −26.982 | 1.00 | 31.97 | 6 | C |
| ATOM | 1833 | CD1 | LEU | B | 25 | −26.211 | 4.186 | −26.338 | 1.00 | 32.19 | 6 | C |
| ATOM | 1834 | CD2 | LEU | B | 25 | −26.852 | 6.594 | −25.919 | 1.00 | 34.03 | 6 | C |
| ATOM | 1835 | C | LEU | B | 25 | −26.333 | 4.927 | −29.928 | 1.00 | 31.62 | 6 | C |
| ATOM | 1836 | O | LEU | B | 25 | −27.015 | 3.904 | −29.895 | 1.00 | 31.80 | 8 | O |
| ATOM | 1837 | N | GLN | B | 26 | −26.619 | 5.949 | −30.726 | 1.00 | 31.03 | 7 | N |
| ATOM | 1838 | CA | GLN | B | 26 | −27.785 | 5.895 | −31.608 | 1.00 | 30.78 | 6 | C |
| ATOM | 1839 | CB | GLN | B | 26 | −27.993 | 7.236 | −32.330 | 1.00 | 31.06 | 6 | C |
| ATOM | 1840 | CG | GLN | B | 26 | −28.570 | 8.351 | −31.445 | 1.00 | 33.92 | 6 | C |
| ATOM | 1841 | CD | GLN | B | 26 | −28.926 | 9.607 | −32.236 | 1.00 | 38.19 | 6 | C |
| ATOM | 1842 | OE1 | GLN | B | 26 | −28.178 | 10.021 | −33.114 | 1.00 | 39.61 | 8 | O |
| ATOM | 1843 | NE2 | GLN | B | 26 | −30.068 | 10.214 | −31.919 | 1.00 | 40.60 | 7 | N |
| ATOM | 1844 | C | GLN | B | 26 | −27.678 | 4.750 | −32.620 | 1.00 | 29.63 | 6 | C |
| ATOM | 1845 | O | GLN | B | 26 | −28.666 | 4.101 | −32.938 | 1.00 | 29.49 | 8 | O |
| ATOM | 1846 | N | GLN | B | 27 | −26.482 | 4.524 | −33.146 | 1.00 | 29.11 | 7 | N |
| ATOM | 1847 | CA | GLN | B | 27 | −26.258 | 3.449 | −34.102 | 1.00 | 28.58 | 6 | C |
| ATOM | 1848 | CB | GLN | B | 27 | −24.844 | 3.560 | −34.694 | 1.00 | 28.95 | 6 | C |
| ATOM | 1849 | CG | GLN | B | 27 | −24.627 | 4.822 | −35.541 | 1.00 | 30.01 | 6 | C |
| ATOM | 1850 | CD | GLN | B | 27 | −23.158 | 5.178 | −35.722 | 1.00 | 31.97 | 6 | C |
| ATOM | 1851 | OE1 | GLN | B | 27 | −22.277 | 4.377 | −35.412 | 1.00 | 30.66 | 8 | O |
| ATOM | 1852 | NE2 | GLN | B | 27 | −22.894 | 6.381 | −36.235 | 1.00 | 30.20 | 7 | N |
| ATOM | 1853 | C | GLN | B | 27 | −26.462 | 2.070 | −33.454 | 1.00 | 28.10 | 6 | C |
| ATOM | 1854 | O | GLN | B | 27 | −27.047 | 1.168 | −34.050 | 1.00 | 27.55 | 8 | O |
| ATOM | 1855 | N | LEU | B | 28 | −25.953 | 1.907 | −32.239 | 1.00 | 27.60 | 7 | N |
| ATOM | 1856 | CA | LEU | B | 28 | −26.105 | 0.640 | −31.534 | 1.00 | 27.94 | 6 | C |
| ATOM | 1857 | CB | LEU | B | 28 | −25.145 | 0.574 | −30.344 | 1.00 | 27.52 | 6 | C |
| ATOM | 1858 | CG | LEU | B | 28 | −23.674 | 0.414 | −30.741 | 1.00 | 27.57 | 6 | C |
| ATOM | 1859 | CD1 | LEU | B | 28 | −22.758 | 0.627 | −29.547 | 1.00 | 29.37 | 6 | C |
| ATOM | 1860 | CD2 | LEU | B | 28 | −23.410 | −0.943 | −31.367 | 1.00 | 28.78 | 6 | C |
| ATOM | 1861 | C | LEU | B | 28 | −27.560 | 0.445 | −31.114 | 1.00 | 28.02 | 6 | C |
| ATOM | 1862 | O | LEU | B | 28 | −28.134 | −0.632 | −31.298 | 1.00 | 28.14 | 8 | O |
| ATOM | 1863 | N | HIS | B | 29 | −28.172 | 1.498 | −30.580 | 1.00 | 28.71 | 7 | N |
| ATOM | 1864 | CA | HIS | B | 29 | −29.567 | 1.411 | −30.178 | 1.00 | 29.41 | 6 | C |
| ATOM | 1865 | CB | HIS | B | 29 | −30.088 | 2.755 | −29.660 | 1.00 | 29.83 | 6 | C |
| ATOM | 1866 | CG | HIS | B | 29 | −31.563 | 2.756 | −29.388 | 1.00 | 31.05 | 6 | C |
| ATOM | 1867 | ND1 | HIS | B | 29 | −32.112 | 2.192 | −28.256 | 1.00 | 33.08 | 7 | N |
| ATOM | 1868 | CE1 | HIS | B | 29 | −33.427 | 2.325 | −28.291 | 1.00 | 33.88 | 6 | C |
| ATOM | 1869 | NE2 | HIS | B | 29 | −33.751 | 2.954 | −29.408 | 1.00 | 33.28 | 7 | N |
| ATOM | 1870 | CD2 | HIS | B | 29 | −32.604 | 3.236 | −30.111 | 1.00 | 33.23 | 6 | C |
| ATOM | 1871 | C | HIS | B | 29 | −30.406 | 0.949 | −31.363 | 1.00 | 29.61 | 6 | C |
| ATOM | 1872 | O | HIS | B | 29 | −31.248 | 0.059 | −31.243 | 1.00 | 29.22 | 8 | O |
| ATOM | 1873 | N | SER | B | 30 | −30.169 | 1.556 | −32.516 | 1.00 | 29.41 | 7 | N |
| ATOM | 1874 | CA | SER | B | 30 | −30.928 | 1.205 | −33.710 | 1.00 | 30.33 | 6 | C |
| ATOM | 1875 | CB | SER | B | 30 | −30.558 | 2.150 | −34.857 | 1.00 | 30.29 | 6 | C |
| ATOM | 1876 | OG | SER | B | 30 | −31.372 | 1.911 | −35.981 | 1.00 | 32.27 | 8 | O |
| ATOM | 1877 | C | SER | B | 30 | −30.747 | −0.256 | −34.145 | 1.00 | 29.67 | 6 | C |
| ATOM | 1878 | O | SER | B | 30 | −31.725 | −0.971 | −34.379 | 1.00 | 29.62 | 8 | O |
| ATOM | 1879 | N | VAL | B | 31 | −29.508 | −0.716 | −34.264 | 1.00 | 29.60 | 7 | N |
| ATOM | 1880 | CA | VAL | B | 31 | −29.321 | −2.091 | −34.727 | 1.00 | 29.79 | 6 | C |
| ATOM | 1881 | CB | VAL | B | 31 | −27.859 | −2.398 | −35.181 | 1.00 | 29.90 | 6 | C |
| ATOM | 1882 | CG1 | VAL | B | 31 | −26.890 | −2.255 | −34.045 | 1.00 | 29.99 | 6 | C |
| ATOM | 1883 | CG2 | VAL | B | 31 | −27.780 | −3.784 | −35.806 | 1.00 | 30.78 | 6 | C |
| ATOM | 1884 | C | VAL | B | 31 | −29.858 | −3.109 | −33.711 | 1.00 | 29.27 | 6 | C |
| ATOM | 1885 | O | VAL | B | 31 | −30.505 | −4.081 | −34.086 | 1.00 | 29.15 | 8 | O |
| ATOM | 1886 | N | ASN | B | 32 | −29.637 | −2.859 | −32.424 | 1.00 | 29.19 | 7 | N |
| ATOM | 1887 | CA | ASN | B | 32 | −30.106 | −3.792 | −31.407 | 1.00 | 29.31 | 6 | C |
| ATOM | 1888 | CB | ASN | B | 32 | −29.550 | −3.434 | −30.021 | 1.00 | 28.38 | 6 | C |
| ATOM | 1889 | CG | ASN | B | 32 | −28.034 | −3.544 | −29.955 | 1.00 | 28.07 | 6 | C |
| ATOM | 1890 | OD1 | ASN | B | 32 | −27.414 | −4.173 | −30.811 | 1.00 | 27.63 | 8 | O |
| ATOM | 1891 | ND2 | ASN | B | 32 | −27.429 | −2.936 | −28.930 | 1.00 | 26.72 | 7 | N |
| ATOM | 1892 | C | ASN | B | 32 | −31.629 | −3.892 | −31.401 | 1.00 | 29.61 | 6 | C |
| ATOM | 1893 | O | ASN | B | 32 | −32.175 | −4.979 | −31.303 | 1.00 | 29.49 | 8 | O |
| ATOM | 1894 | N | ALA | B | 33 | −32.305 | −2.752 | −31.535 | 1.00 | 30.63 | 7 | N |
| ATOM | 1895 | CA | ALA | B | 33 | −33.768 | −2.699 | −31.534 | 1.00 | 31.08 | 6 | C |
| ATOM | 1896 | CB | ALA | B | 33 | −34.242 | −1.259 | −31.578 | 1.00 | 31.16 | 6 | C |
| ATOM | 1897 | C | ALA | B | 33 | −34.396 | −3.513 | −32.676 | 1.00 | 31.77 | 6 | C |
| ATOM | 1898 | O | ALA | B | 33 | −35.542 | −3.989 | −32.564 | 1.00 | 31.28 | 8 | O |
| ATOM | 1899 | N | SER | B | 34 | −33.642 | −3.696 | −33.759 | 1.00 | 31.76 | 7 | N |
| ATOM | 1900 | CA | SER | B | 34 | −34.128 | −4.482 | −34.889 | 1.00 | 32.48 | 6 | C |
| ATOM | 1901 | CB | SER | B | 34 | −33.389 | −4.092 | −36.177 | 1.00 | 32.31 | 6 | C |
| ATOM | 1902 | OG | SER | B | 34 | −32.074 | −4.628 | −36.186 | 1.00 | 31.09 | 8 | O |
| ATOM | 1903 | C | SER | B | 34 | −33.992 | −5.993 | −34.655 | 1.00 | 33.19 | 6 | C |
| ATOM | 1904 | O | SER | B | 34 | −34.454 | −6.791 | −35.479 | 1.00 | 33.37 | 8 | O |
| ATOM | 1905 | N | LYS | B | 35 | −33.370 | −6.368 | −33.535 | 1.00 | 33.91 | 7 | N |
| ATOM | 1906 | CA | LYS | B | 35 | −33.127 | −7.769 | −33.169 | 1.00 | 34.68 | 6 | C |
| ATOM | 1907 | CB | LYS | B | 35 | −34.403 | −8.404 | −32.615 | 1.00 | 35.29 | 6 | C |
| ATOM | 1908 | CG | LYS | B | 35 | −35.058 | −7.602 | −31.485 | 1.00 | 36.72 | 6 | C |
| ATOM | 1909 | CD | LYS | B | 35 | −34.692 | −8.140 | −30.122 | 1.00 | 40.57 | 6 | C |
| ATOM | 1910 | CE | LYS | B | 35 | −35.496 | −7.457 | −29.015 | 1.00 | 40.76 | 6 | C |
| ATOM | 1911 | NZ | LYS | B | 35 | −36.831 | −8.105 | −28.810 | 1.00 | 42.80 | 7 | N |
| ATOM | 1912 | C | LYS | B | 35 | −32.630 | −8.572 | −34.366 | 1.00 | 34.80 | 6 | C |
| ATOM | 1913 | O | LYS | B | 35 | −33.317 | −9.475 | −34.844 | 1.00 | 34.35 | 8 | O |
| ATOM | 1914 | N | PRO | B | 36 | −31.430 | −8.253 | −34.837 | 1.00 | 35.09 | 7 | N |
| ATOM | 1915 | CA | PRO | B | 36 | −30.903 | −8.840 | −36.077 | 1.00 | 35.28 | 6 | C |
| ATOM | 1916 | CB | PRO | B | 36 | −29.565 | −8.116 | −36.256 | 1.00 | 35.30 | 6 | C |
| ATOM | 1917 | CG | PRO | B | 36 | −29.192 | −7.714 | −34.847 | 1.00 | 35.62 | 6 | C |
| ATOM | 1918 | CD | PRO | B | 36 | −30.496 | −7.276 | −34.249 | 1.00 | 34.89 | 6 | C |
| ATOM | 1919 | C | PRO | B | 36 | −30.705 | −10.360 | −36.077 | 1.00 | 35.65 | 6 | C |
| ATOM | 1920 | O | PRO | B | 36 | −30.595 | −10.922 | −37.167 | 1.00 | 35.42 | 8 | O |
| ATOM | 1921 | N | SER | B | 37 | −30.662 | −11.017 | −34.916 | 1.00 | 35.73 | 7 | N |
| ATOM | 1922 | CA | SER | B | 37 | −30.474 | −12.469 | −34.915 | 1.00 | 36.28 | 6 | C |
| ATOM | 1923 | CB | SER | B | 37 | −29.538 | −12.929 | −33.789 | 1.00 | 36.41 | 6 | C |
| ATOM | 1924 | OG | SER | B | 37 | −30.183 | −12.868 | −32.533 | 1.00 | 35.91 | 8 | O |
| ATOM | 1925 | C | SER | B | 37 | −31.789 | −13.239 | −34.869 | 1.00 | 36.95 | 6 | C |
| ATOM | 1926 | O | SER | B | 37 | −31.803 | −14.464 | −34.989 | 1.00 | 37.58 | 8 | O |
| ATOM | 1927 | N | GLU | B | 38 | −32.893 | −12.524 | −34.699 | 1.00 | 37.36 | 7 | N |
| ATOM | 1928 | CA | GLU | B | 38 | −34.199 | −13.161 | −34.657 | 1.00 | 38.54 | 6 | C |
| ATOM | 1929 | CB | GLU | B | 38 | −35.069 | −12.541 | −33.555 | 1.00 | 38.20 | 6 | C |
| ATOM | 1930 | CG | GLU | B | 38 | −34.497 | −12.752 | −32.162 | 1.00 | 39.65 | 6 | C |
| ATOM | 1931 | CD | GLU | B | 38 | −35.307 | −12.080 | −31.061 | 1.00 | 41.33 | 6 | C |
| ATOM | 1932 | OE1 | GLU | B | 38 | −36.512 | −11.811 | −31.263 | 1.00 | 41.59 | 8 | O |
| ATOM | 1933 | OE2 | GLU | B | 38 | −34.733 | −11.822 | −29.983 | 1.00 | 42.51 | 8 | O |
| ATOM | 1934 | C | GLU | B | 38 | −34.866 | −13.032 | −36.018 | 1.00 | 38.82 | 6 | C |
| ATOM | 1935 | O | GLU | B | 38 | −35.934 | −12.459 | −36.143 | 1.00 | 39.40 | 8 | O |
| ATOM | 1936 | N | ARG | B | 39 | −34.213 | −13.558 | −37.043 | 1.00 | 39.56 | 7 | N |
| ATOM | 1937 | CA | ARG | B | 39 | −34.746 | −13.508 | −38.395 | 1.00 | 40.06 | 6 | C |
| ATOM | 1938 | CB | ARG | B | 39 | −33.852 | −12.652 | −39.288 | 1.00 | 39.74 | 6 | C |
| ATOM | 1939 | CG | ARG | B | 39 | −33.605 | −11.249 | −38.760 | 1.00 | 38.73 | 6 | C |
| ATOM | 1940 | CD | ARG | B | 39 | −34.740 | −10.274 | −39.009 | 1.00 | 36.27 | 6 | C |
| ATOM | 1941 | NE | ARG | B | 39 | −34.464 | −8.983 | −38.391 | 1.00 | 34.61 | 7 | N |
| ATOM | 1942 | CZ | ARG | B | 39 | −33.754 | −8.019 | −38.963 | 1.00 | 35.62 | 6 | C |
| ATOM | 1943 | NH1 | ARG | B | 39 | −33.256 | −8.188 | −40.186 | 1.00 | 34.56 | 7 | N |
| ATOM | 1944 | NH2 | ARG | B | 39 | −33.550 | −6.876 | −38.318 | 1.00 | 34.83 | 7 | N |
| ATOM | 1945 | C | ARG | B | 39 | −34.796 | −14.920 | −38.945 | 1.00 | 40.94 | 6 | C |
| ATOM | 1946 | O | ARG | B | 39 | −33.995 | −15.768 | −38.558 | 1.00 | 41.00 | 8 | O |
| ATOM | 1947 | N | GLY | B | 40 | −35.742 | −15.175 | −39.843 | 1.00 | 41.86 | 7 | N |
| ATOM | 1948 | CA | GLY | B | 40 | −35.849 | −16.479 | −40.464 | 1.00 | 42.75 | 6 | C |
| ATOM | 1949 | C | GLY | B | 40 | −34.620 | −16.743 | −41.309 | 1.00 | 43.43 | 6 | C |
| ATOM | 1950 | O | GLY | B | 40 | −33.996 | −17.798 | −41.210 | 1.00 | 44.09 | 8 | O |
| ATOM | 1951 | N | LEU | B | 41 | −34.265 | −15.773 | −42.142 | 1.00 | 43.47 | 7 | N |
| ATOM | 1952 | CA | LEU | B | 41 | −33.093 | −15.910 | −42.992 | 1.00 | 43.77 | 6 | C |
| ATOM | 1953 | CB | LEU | B | 41 | −33.485 | −15.855 | −44.473 | 1.00 | 43.92 | 6 | C |
| ATOM | 1954 | CG | LEU | B | 41 | −32.312 | −15.792 | −45.454 | 1.00 | 45.32 | 6 | C |
| ATOM | 1955 | CD1 | LEU | B | 41 | −31.384 | −16.988 | −45.271 | 1.00 | 46.78 | 6 | C |
| ATOM | 1956 | CD2 | LEU | B | 41 | −32.814 | −15.720 | −46.887 | 1.00 | 46.32 | 6 | C |
| ATOM | 1957 | C | LEU | B | 41 | −32.071 | −14.827 | −42.675 | 1.00 | 43.35 | 6 | C |
| ATOM | 1958 | O | LEU | B | 41 | −32.352 | −13.638 | −42.805 | 1.00 | 43.80 | 8 | O |
| ATOM | 1959 | N | VAL | B | 42 | −30.890 | −15.249 | −42.242 | 1.00 | 42.64 | 7 | N |
| ATOM | 1960 | CA | VAL | B | 42 | −29.816 | −14.325 | −41.922 | 1.00 | 42.03 | 6 | C |
| ATOM | 1961 | CB | VAL | B | 42 | −29.002 | −14.809 | −40.696 | 1.00 | 42.22 | 6 | C |
| ATOM | 1962 | CG1 | VAL | B | 42 | −27.760 | −13.961 | −40.511 | 1.00 | 41.23 | 6 | C |
| ATOM | 1963 | CG2 | VAL | B | 42 | −29.853 | −14.784 | −39.434 | 1.00 | 42.53 | 6 | C |
| ATOM | 1964 | C | VAL | B | 42 | −28.882 | −14.217 | −43.119 | 1.00 | 41.54 | 6 | C |
| ATOM | 1965 | O | VAL | B | 42 | −28.497 | −15.233 | −43.691 | 1.00 | 41.43 | 8 | O |
| ATOM | 1966 | N | ARG | B | 43 | −28.540 | −12.994 | −43.513 | 1.00 | 40.84 | 7 | N |
| ATOM | 1967 | CA | ARG | B | 43 | −27.606 | −12.783 | −44.620 | 1.00 | 40.38 | 6 | C |
| ATOM | 1968 | CB | ARG | B | 43 | −28.335 | −12.301 | −45.876 | 1.00 | 40.78 | 6 | C |
| ATOM | 1969 | CG | ARG | B | 43 | −29.070 | −13.411 | −46.622 | 1.00 | 43.34 | 6 | C |
| ATOM | 1970 | CD | ARG | B | 43 | −29.934 | −12.922 | −47.784 | 1.00 | 46.71 | 6 | C |
| ATOM | 1971 | NE | ARG | B | 43 | −31.124 | −12.220 | −47.306 | 1.00 | 50.29 | 7 | N |
| ATOM | 1972 | CZ | ARG | B | 43 | −31.778 | −11.288 | −47.995 | 1.00 | 51.44 | 6 | C |
| ATOM | 1973 | NH1 | ARG | B | 43 | −31.363 | −10.939 | −49.204 | 1.00 | 51.72 | 7 | N |
| ATOM | 1974 | NH2 | ARG | B | 43 | −32.850 | −10.704 | −47.469 | 1.00 | 52.75 | 7 | N |
| ATOM | 1975 | C | ARG | B | 43 | −26.512 | −11.803 | −44.205 | 1.00 | 39.52 | 6 | C |
| ATOM | 1976 | O | ARG | B | 43 | −26.335 | −10.743 | −44.809 | 1.00 | 38.70 | 8 | O |
| ATOM | 1977 | N | GLN | B | 44 | −25.778 | −12.187 | −43.166 | 1.00 | 38.29 | 7 | N |
| ATOM | 1978 | CA | GLN | B | 44 | −24.723 | −11.364 | −42.592 | 1.00 | 37.43 | 6 | C |
| ATOM | 1979 | CB | GLN | B | 44 | −24.009 | −12.154 | −41.498 | 1.00 | 37.56 | 6 | C |
| ATOM | 1980 | CG | GLN | B | 44 | −23.236 | −11.321 | −40.507 | 1.00 | 39.18 | 6 | C |
| ATOM | 1981 | CD | GLN | B | 44 | −22.846 | −12.139 | −39.295 | 1.00 | 40.77 | 6 | C |
| ATOM | 1982 | OE1 | GLN | B | 44 | −23.595 | −13.024 | −38.892 | 1.00 | 41.02 | 8 | O |
| ATOM | 1983 | NE2 | GLN | B | 44 | −21.674 | −11.863 | −38.726 | 1.00 | 41.75 | 7 | N |
| ATOM | 1984 | C | GLN | B | 44 | −23.715 | −10.889 | −43.635 | 1.00 | 36.43 | 6 | C |
| ATOM | 1985 | O | GLN | B | 44 | −23.247 | −9.750 | −43.585 | 1.00 | 35.59 | 8 | O |
| ATOM | 1986 | N | ALA | B | 45 | −23.390 | −11.762 | −44.585 | 1.00 | 35.69 | 7 | N |
| ATOM | 1987 | CA | ALA | B | 45 | −22.416 | −11.419 | −45.614 | 1.00 | 35.09 | 6 | C |
| ATOM | 1988 | CB | ALA | B | 45 | −22.193 | −12.599 | −46.567 | 1.00 | 35.40 | 6 | C |
| ATOM | 1989 | C | ALA | B | 45 | −22.774 | −10.149 | −46.395 | 1.00 | 34.46 | 6 | C |
| ATOM | 1990 | O | ALA | B | 45 | −21.879 | −9.416 | −46.816 | 1.00 | 34.15 | 8 | O |
| ATOM | 1991 | N | GLU | B | 46 | −24.068 | −9.882 | −46.575 | 1.00 | 33.79 | 7 | N |
| ATOM | 1992 | CA | GLU | B | 46 | −24.499 | −8.700 | −47.329 | 1.00 | 33.73 | 6 | C |
| ATOM | 1993 | CB | GLU | B | 46 | −25.999 | −8.765 | −47.662 | 1.00 | 33.80 | 6 | C |
| ATOM | 1994 | CG | GLU | B | 46 | −26.421 | −9.857 | −48.641 | 1.00 | 35.36 | 6 | C |
| ATOM | 1995 | CD | GLU | B | 46 | −25.928 | −9.626 | −50.060 | 1.00 | 37.74 | 6 | C |
| ATOM | 1996 | OE1 | GLU | B | 46 | −25.631 | −8.468 | −50.428 | 1.00 | 37.79 | 8 | O |
| ATOM | 1997 | OE2 | GLU | B | 46 | −25.832 | −10.620 | −50.814 | 1.00 | 39.71 | 8 | O |
| ATOM | 1998 | C | GLU | B | 46 | −24.220 | −7.389 | −46.596 | 1.00 | 33.38 | 6 | C |
| ATOM | 1999 | O | GLU | B | 46 | −24.357 | −6.307 | −47.177 | 1.00 | 32.62 | 8 | O |
| ATOM | 2000 | N | ALA | B | 47 | −23.864 | −7.481 | −45.314 | 1.00 | 32.85 | 7 | N |
| ATOM | 2001 | CA | ALA | B | 47 | −23.568 | −6.290 | −44.528 | 1.00 | 33.07 | 6 | C |
| ATOM | 2002 | CB | ALA | B | 47 | −24.142 | −6.413 | −43.113 | 1.00 | 32.94 | 6 | C |
| ATOM | 2003 | C | ALA | B | 47 | −22.073 | −5.995 | −44.471 | 1.00 | 33.26 | 6 | C |
| ATOM | 2004 | O | ALA | B | 47 | −21.660 | −4.966 | −43.941 | 1.00 | 32.82 | 8 | O |
| ATOM | 2005 | N | GLU | B | 48 | −21.265 | −6.902 | −45.007 | 1.00 | 33.94 | 7 | N |
| ATOM | 2006 | CA | GLU | B | 48 | −19.821 | −6.705 | −45.013 | 1.00 | 35.23 | 6 | C |
| ATOM | 2007 | CB | GLU | B | 48 | −19.107 | −7.975 | −45.482 | 1.00 | 35.50 | 6 | C |
| ATOM | 2008 | CG | GLU | B | 48 | −19.178 | −9.119 | −44.489 | 1.00 | 37.08 | 6 | C |
| ATOM | 2009 | CD | GLU | B | 48 | −18.470 | −10.365 | −44.981 | 1.00 | 39.75 | 6 | C |
| ATOM | 2010 | OE1 | GLU | B | 48 | −17.398 | −10.228 | −45.611 | 1.00 | 42.17 | 8 | O |
| ATOM | 2011 | OE2 | GLU | B | 48 | −18.981 | −11.476 | −44.734 | 1.00 | 39.89 | 8 | O |
| ATOM | 2012 | C | GLU | B | 48 | −19.413 | −5.532 | −45.899 | 1.00 | 35.92 | 6 | C |
| ATOM | 2013 | O | GLU | B | 48 | −19.998 | −5.311 | −46.953 | 1.00 | 35.83 | 8 | O |
| ATOM | 2014 | N | ASP | B | 49 | −18.406 | −4.783 | −45.464 | 1.00 | 36.95 | 7 | N |
| ATOM | 2015 | CA | ASP | B | 49 | −17.895 | −3.667 | −46.247 | 1.00 | 38.46 | 6 | C |
| ATOM | 2016 | CB | ASP | B | 49 | −18.668 | −2.378 | −45.949 | 1.00 | 38.28 | 6 | C |
| ATOM | 2017 | CG | ASP | B | 49 | −18.434 | −1.306 | −46.997 | 1.00 | 39.03 | 6 | C |
| ATOM | 2018 | OD1 | ASP | B | 49 | −17.482 | −1.460 | −47.788 | 1.00 | 39.15 | 8 | O |
| ATOM | 2019 | OD2 | ASP | B | 49 | −19.143 | −0.282 | −47.113 | 1.00 | 39.57 | 8 | O |
| ATOM | 2020 | C | ASP | B | 49 | −16.403 | −3.482 | −45.977 | 1.00 | 39.53 | 6 | C |
| ATOM | 2021 | O | ASP | B | 49 | −16.014 | −2.811 | −45.024 | 1.00 | 39.07 | 8 | O |
| ATOM | 2022 | N | PRO | B | 50 | −15.581 | −4.100 | −46.818 | 1.00 | 41.19 | 7 | N |
| ATOM | 2023 | CA | PRO | B | 50 | −14.114 | −4.027 | −46.710 | 1.00 | 42.47 | 6 | C |
| ATOM | 2024 | CB | PRO | B | 50 | −13.637 | −4.691 | −48.005 | 1.00 | 42.40 | 6 | C |
| ATOM | 2025 | CG | PRO | B | 50 | −14.750 | −5.607 | −48.395 | 1.00 | 42.26 | 6 | C |
| ATOM | 2026 | CD | PRO | B | 50 | −16.017 | −4.935 | −47.951 | 1.00 | 41.29 | 6 | C |
| ATOM | 2027 | C | PRO | B | 50 | −13.559 | −2.605 | −46.632 | 1.00 | 43.64 | 6 | C |
| ATOM | 2028 | O | PRO | B | 50 | −12.559 | −2.378 | −45.949 | 1.00 | 44.29 | 8 | O |
| ATOM | 2029 | N | ALA | B | 51 | −14.189 | −1.663 | −47.322 | 1.00 | 45.00 | 7 | N |
| ATOM | 2030 | CA | ALA | B | 51 | −13.724 | −0.278 | −47.311 | 1.00 | 45.79 | 6 | C |
| ATOM | 2031 | CB | ALA | B | 51 | −14.465 | 0.533 | −48.344 | 1.00 | 46.07 | 6 | C |
| ATOM | 2032 | C | ALA | B | 51 | −13.897 | 0.348 | −45.939 | 1.00 | 46.52 | 6 | C |
| ATOM | 2033 | O | ALA | B | 51 | −13.550 | 1.514 | −45.726 | 1.00 | 46.62 | 8 | O |
| ATOM | 2034 | N | CYS | B | 52 | −14.424 | −0.442 | −45.008 | 1.00 | 46.79 | 7 | N |
| ATOM | 2035 | CA | CYS | B | 52 | −14.698 | 0.031 | −43.662 | 1.00 | 47.44 | 6 | C |
| ATOM | 2036 | CB | CYS | B | 52 | −16.079 | −0.430 | −43.223 | 1.00 | 47.69 | 6 | C |
| ATOM | 2037 | SG | CYS | B | 52 | −17.315 | 0.800 | −43.562 | 1.00 | 50.99 | 16 | S |
| ATOM | 2038 | C | CYS | B | 52 | −13.702 | −0.439 | −42.633 | 1.00 | 46.96 | 6 | C |
| ATOM | 2039 | O | CYS | B | 52 | −13.770 | −0.026 | −41.475 | 1.00 | 46.95 | 8 | O |
| ATOM | 2040 | N | ILE | B | 53 | −12.809 | −1.334 | −43.034 | 1.00 | 46.55 | 7 | N |
| ATOM | 2041 | CA | ILE | B | 53 | −11.809 | −1.825 | −42.107 | 1.00 | 46.54 | 6 | C |
| ATOM | 2042 | CB | ILE | B | 53 | −10.772 | −2.698 | −42.833 | 1.00 | 46.69 | 6 | C |
| ATOM | 2043 | CG1 | ILE | B | 53 | −11.474 | −3.857 | −43.543 | 1.00 | 47.41 | 6 | C |
| ATOM | 2044 | CD1 | ILE | B | 53 | −10.540 | −4.758 | −44.349 | 1.00 | 47.75 | 6 | C |
| ATOM | 2045 | CG2 | ILE | B | 53 | −9.743 | −3.236 | −41.849 | 1.00 | 46.72 | 6 | C |
| ATOM | 2046 | C | ILE | B | 53 | −11.170 | −0.604 | −41.462 | 1.00 | 46.15 | 6 | C |
| ATOM | 2047 | O | ILE | B | 53 | −10.792 | 0.340 | −42.151 | 1.00 | 46.05 | 8 | O |
| ATOM | 2048 | N | PRO | B | 54 | −11.109 | −0.600 | −40.137 | 1.00 | 46.00 | 7 | N |
| ATOM | 2049 | CA | PRO | B | 54 | −10.550 | 0.525 | −39.384 | 1.00 | 45.97 | 6 | C |
| ATOM | 2050 | CB | PRO | B | 54 | −10.600 | 0.025 | −37.938 | 1.00 | 45.87 | 6 | C |
| ATOM | 2051 | CG | PRO | B | 54 | −10.707 | −1.466 | −38.075 | 1.00 | 46.07 | 6 | C |
| ATOM | 2052 | CD | PRO | B | 54 | −11.613 | −1.657 | −39.244 | 1.00 | 45.94 | 6 | C |
| ATOM | 2053 | C | PRO | B | 54 | −9.114 | 0.854 | −39.779 | 1.00 | 46.00 | 6 | C |
| ATOM | 2054 | O | PRO | B | 54 | −8.362 | −0.018 | −40.220 | 1.00 | 46.11 | 8 | O |
| ATOM | 2055 | N | ILE | B | 55 | −8.747 | 2.120 | −39.637 | 1.00 | 45.93 | 7 | N |
| ATOM | 2056 | CA | ILE | B | 55 | −7.383 | 2.536 | −39.926 | 1.00 | 46.00 | 6 | C |
| ATOM | 2057 | CB | ILE | B | 55 | −7.316 | 4.056 | −40.160 | 1.00 | 46.03 | 6 | C |
| ATOM | 2058 | CG1 | ILE | B | 55 | −8.341 | 4.480 | −41.213 | 1.00 | 46.99 | 6 | C |
| ATOM | 2059 | CD1 | ILE | B | 55 | −8.567 | 5.979 | −41.276 | 1.00 | 47.02 | 6 | C |
| ATOM | 2060 | CG2 | ILE | B | 55 | −5.921 | 4.469 | −40.603 | 1.00 | 46.63 | 6 | C |
| ATOM | 2061 | C | ILE | B | 55 | −6.509 | 2.133 | −38.742 | 1.00 | 45.49 | 6 | C |
| ATOM | 2062 | O | ILE | B | 55 | −5.387 | 1.655 | −38.921 | 1.00 | 45.69 | 8 | O |
| ATOM | 2063 | N | PHE | B | 56 | −7.048 | 2.294 | −37.536 | 1.00 | 44.71 | 7 | N |
| ATOM | 2064 | CA | PHE | B | 56 | −6.321 | 1.969 | −36.312 | 1.00 | 44.06 | 6 | C |
| ATOM | 2065 | CB | PHE | B | 56 | −5.940 | 3.250 | −35.561 | 1.00 | 44.46 | 6 | C |
| ATOM | 2066 | CG | PHE | B | 56 | −5.109 | 4.209 | −36.357 | 1.00 | 45.29 | 6 | C |
| ATOM | 2067 | CD1 | PHE | B | 56 | −5.662 | 5.375 | −36.854 | 1.00 | 46.35 | 6 | C |
| ATOM | 2068 | CE1 | PHE | B | 56 | −4.893 | 6.274 | −37.576 | 1.00 | 46.75 | 6 | C |
| ATOM | 2069 | CZ | PHE | B | 56 | −3.557 | 6.008 | −37.807 | 1.00 | 46.64 | 6 | C |
| ATOM | 2070 | CE2 | PHE | B | 56 | −2.993 | 4.850 | −37.311 | 1.00 | 46.33 | 6 | C |
| ATOM | 2071 | CD2 | PHE | B | 56 | −3.766 | 3.958 | −36.589 | 1.00 | 46.39 | 6 | C |
| ATOM | 2072 | C | PHE | B | 56 | −7.125 | 1.112 | −35.338 | 1.00 | 43.15 | 6 | C |
| ATOM | 2073 | O | PHE | B | 56 | −8.349 | 1.247 | −35.242 | 1.00 | 42.86 | 8 | O |
| ATOM | 2074 | N | TRP | B | 57 | −6.422 | 0.240 | −34.617 | 1.00 | 41.94 | 7 | N |
| ATOM | 2075 | CA | TRP | B | 57 | −6.995 | −0.506 | −33.496 | 1.00 | 41.32 | 6 | C |
| ATOM | 2076 | CB | TRP | B | 57 | −7.742 | −1.778 | −33.932 | 1.00 | 40.91 | 6 | C |
| ATOM | 2077 | CG | TRP | B | 57 | −6.895 | −2.795 | −34.636 | 1.00 | 39.37 | 6 | C |
| ATOM | 2078 | CD1 | TRP | B | 57 | −6.168 | −3.799 | −34.069 | 1.00 | 39.14 | 6 | C |
| ATOM | 2079 | NE1 | TRP | B | 57 | −5.524 | −4.528 | −35.042 | 1.00 | 38.59 | 7 | N |
| ATOM | 2080 | CE2 | TRP | B | 57 | −5.840 | −4.002 | −36.268 | 1.00 | 39.25 | 6 | C |
| ATOM | 2081 | CD2 | TRP | B | 57 | −6.705 | −2.912 | −36.046 | 1.00 | 38.79 | 6 | C |
| ATOM | 2082 | CE3 | TRP | B | 57 | −7.175 | −2.197 | −37.153 | 1.00 | 38.60 | 6 | C |
| ATOM | 2083 | CZ3 | TRP | B | 57 | −6.779 | −2.588 | −38.420 | 1.00 | 39.95 | 6 | C |
| ATOM | 2084 | CH2 | TRP | B | 57 | −5.912 | −3.673 | −38.603 | 1.00 | 39.37 | 6 | C |
| ATOM | 2085 | CZ2 | TRP | B | 57 | −5.435 | −4.390 | −37.543 | 1.00 | 39.00 | 6 | C |
| ATOM | 2086 | C | TRP | B | 57 | −5.890 | −0.836 | −32.493 | 1.00 | 41.24 | 6 | C |
| ATOM | 2087 | O | TRP | B | 57 | −4.708 | −0.772 | −32.824 | 1.00 | 41.34 | 8 | O |
| ATOM | 2088 | N | VAL | B | 58 | −6.277 | −1.167 | −31.267 | 1.00 | 40.90 | 7 | N |
| ATOM | 2089 | CA | VAL | B | 58 | −5.322 | −1.524 | −30.229 | 1.00 | 40.95 | 6 | C |
| ATOM | 2090 | CB | VAL | B | 58 | −5.938 | −1.346 | −28.827 | 1.00 | 40.92 | 6 | C |
| ATOM | 2091 | CG1 | VAL | B | 58 | −4.980 | −1.810 | −27.745 | 1.00 | 41.17 | 6 | C |
| ATOM | 2092 | CG2 | VAL | B | 58 | −6.335 | 0.112 | −28.606 | 1.00 | 41.00 | 6 | C |
| ATOM | 2093 | C | VAL | B | 58 | −4.852 | −2.967 | −30.424 | 1.00 | 40.88 | 6 | C |
| ATOM | 2094 | O | VAL | B | 58 | −5.644 | −3.906 | −30.328 | 1.00 | 40.63 | 8 | O |
| ATOM | 2095 | N | SER | B | 59 | −3.562 | −3.137 | −30.710 | 1.00 | 40.71 | 7 | N |
| ATOM | 2096 | CA | SER | B | 59 | −3.004 | −4.469 | −30.963 | 1.00 | 40.82 | 6 | C |
| ATOM | 2097 | CB | SER | B | 59 | −1.917 | −4.400 | −32.039 | 1.00 | 41.06 | 6 | C |
| ATOM | 2098 | OG | SER | B | 59 | −1.090 | −3.266 | −31.856 | 1.00 | 41.44 | 8 | O |
| ATOM | 2099 | C | SER | B | 59 | −2.473 | −5.163 | −29.708 | 1.00 | 40.52 | 6 | C |
| ATOM | 2100 | O | SER | B | 59 | −2.444 | −6.395 | −29.631 | 1.00 | 40.41 | 8 | O |
| ATOM | 2101 | N | LYS | B | 60 | −2.046 | −4.362 | −28.738 | 1.00 | 39.92 | 7 | N |
| ATOM | 2102 | CA | LYS | B | 60 | −1.557 | −4.854 | −27.459 | 1.00 | 39.51 | 6 | C |
| ATOM | 2103 | CB | LYS | B | 60 | −0.055 | −5.160 | −27.518 | 1.00 | 39.60 | 6 | C |
| ATOM | 2104 | CG | LYS | B | 60 | 0.448 | −5.788 | −28.809 | 1.00 | 40.15 | 6 | C |
| ATOM | 2105 | CD | LYS | B | 60 | 1.919 | −6.195 | −28.675 | 1.00 | 41.00 | 6 | C |
| ATOM | 2106 | CE | LYS | B | 60 | 2.458 | −6.765 | −29.982 | 1.00 | 41.88 | 6 | C |
| ATOM | 2107 | NZ | LYS | B | 60 | 3.869 | −7.271 | −29.822 | 1.00 | 42.94 | 7 | N |
| ATOM | 2108 | C | LYS | B | 60 | −1.776 | −3.763 | −26.414 | 1.00 | 39.09 | 6 | C |
| ATOM | 2109 | O | LYS | B | 60 | −1.808 | −2.578 | −26.745 | 1.00 | 38.83 | 8 | O |
| ATOM | 2110 | N | TRP | B | 61 | −1.929 | −4.167 | −25.161 | 1.00 | 38.91 | 7 | N |
| ATOM | 2111 | CA | TRP | B | 61 | −2.053 | −3.211 | −24.063 | 1.00 | 39.28 | 6 | C |
| ATOM | 2112 | CB | TRP | B | 61 | −3.510 | −2.759 | −23.878 | 1.00 | 38.78 | 6 | C |
| ATOM | 2113 | CG | TRP | B | 61 | −4.472 | −3.888 | −23.641 | 1.00 | 37.70 | 6 | C |
| ATOM | 2114 | CD1 | TRP | B | 61 | −5.204 | −4.554 | −24.586 | 1.00 | 35.95 | 6 | C |
| ATOM | 2115 | NE1 | TRP | B | 61 | −5.973 | −5.524 | −23.992 | 1.00 | 36.01 | 7 | N |
| ATOM | 2116 | CE2 | TRP | B | 61 | −5.754 | −5.499 | −22.641 | 1.00 | 36.60 | 6 | C |
| ATOM | 2117 | CD2 | TRP | B | 61 | −4.814 | −4.479 | −22.385 | 1.00 | 36.92 | 6 | C |
| ATOM | 2118 | CE3 | TRP | B | 61 | −4.426 | −4.248 | −21.060 | 1.00 | 38.31 | 6 | C |
| ATOM | 2119 | CZ3 | TRP | B | 61 | −4.966 | −5.036 | −20.061 | 1.00 | 37.79 | 6 | C |
| ATOM | 2120 | CH2 | TRP | B | 61 | −5.893 | −6.038 | −20.351 | 1.00 | 38.23 | 6 | C |
| ATOM | 2121 | CZ2 | TRP | B | 61 | −6.298 | −6.286 | −21.633 | 1.00 | 36.55 | 6 | C |
| ATOM | 2122 | C | TRP | B | 61 | −1.501 | −3.776 | −22.758 | 1.00 | 39.88 | 6 | C |
| ATOM | 2123 | O | TRP | B | 61 | −1.449 | −4.993 | −22.566 | 1.00 | 40.11 | 8 | O |
| ATOM | 2124 | N | VAL | B | 62 | −1.084 | −2.877 | −21.871 | 1.00 | 40.59 | 7 | N |
| ATOM | 2125 | CA | VAL | B | 62 | −0.560 | −3.245 | −20.563 | 1.00 | 41.45 | 6 | C |
| ATOM | 2126 | CB | VAL | B | 62 | 0.977 | −3.140 | −20.510 | 1.00 | 41.29 | 6 | C |
| ATOM | 2127 | CG1 | VAL | B | 62 | 1.480 | −3.465 | −19.115 | 1.00 | 41.94 | 6 | C |
| ATOM | 2128 | CG2 | VAL | B | 62 | 1.615 | −4.067 | −21.519 | 1.00 | 41.97 | 6 | C |
| ATOM | 2129 | C | VAL | B | 62 | −1.156 | −2.297 | −19.524 | 1.00 | 41.74 | 6 | C |
| ATOM | 2130 | O | VAL | B | 62 | −0.977 | −1.081 | −19.612 | 1.00 | 41.59 | 8 | O |
| ATOM | 2131 | N | ASP | B | 63 | −1.863 | −2.861 | −18.551 | 1.00 | 42.53 | 7 | N |
| ATOM | 2132 | CA | ASP | B | 63 | −2.522 | −2.083 | −17.512 | 1.00 | 43.68 | 6 | C |
| ATOM | 2133 | CB | ASP | B | 63 | −3.831 | −2.755 | −17.093 | 1.00 | 43.55 | 6 | C |
| ATOM | 2134 | CG | ASP | B | 63 | −4.525 | −2.030 | −15.956 | 1.00 | 43.95 | 6 | C |
| ATOM | 2135 | OD1 | ASP | B | 63 | −4.019 | −0.969 | −15.526 | 1.00 | 43.59 | 8 | O |
| ATOM | 2136 | OD2 | ASP | B | 63 | −5.576 | −2.450 | −15.421 | 1.00 | 43.71 | 8 | O |
| ATOM | 2137 | C | ASP | B | 63 | −1.632 | −1.893 | −16.287 | 1.00 | 44.67 | 6 | C |
| ATOM | 2138 | O | ASP | B | 63 | −1.605 | −2.734 | −15.387 | 1.00 | 44.34 | 8 | O |
| ATOM | 2139 | N | TYR | B | 64 | −0.905 | −0.784 | −16.267 | 1.00 | 46.03 | 7 | N |
| ATOM | 2140 | CA | TYR | B | 64 | −0.073 | −0.445 | −15.120 | 1.00 | 47.40 | 6 | C |
| ATOM | 2141 | CB | TYR | B | 64 | 1.380 | −0.222 | −15.540 | 1.00 | 47.82 | 6 | C |
| ATOM | 2142 | CG | TYR | B | 64 | 2.167 | −1.506 | −15.659 | 1.00 | 50.03 | 6 | C |
| ATOM | 2143 | CD1 | TYR | B | 64 | 3.478 | −1.504 | −16.112 | 1.00 | 51.96 | 6 | C |
| ATOM | 2144 | CE1 | TYR | B | 64 | 4.197 | −2.688 | −16.220 | 1.00 | 53.33 | 6 | C |
| ATOM | 2145 | CZ | TYR | B | 64 | 3.602 | −3.884 | −15.867 | 1.00 | 53.34 | 6 | C |
| ATOM | 2146 | OH | TYR | B | 64 | 4.307 | −5.062 | −15.968 | 1.00 | 54.77 | 8 | O |
| ATOM | 2147 | CE2 | TYR | B | 64 | 2.305 | −3.907 | −15.412 | 1.00 | 52.75 | 6 | C |
| ATOM | 2148 | CD2 | TYR | B | 64 | 1.596 | −2.725 | −15.311 | 1.00 | 51.72 | 6 | C |
| ATOM | 2149 | C | TYR | B | 64 | −0.642 | 0.802 | −14.462 | 1.00 | 47.57 | 6 | C |
| ATOM | 2150 | O | TYR | B | 64 | 0.101 | 1.633 | −13.947 | 1.00 | 47.39 | 8 | O |
| ATOM | 2151 | N | SER | B | 65 | −1.968 | 0.923 | −14.501 | 1.00 | 47.80 | 7 | N |
| ATOM | 2152 | CA | SER | B | 65 | −2.670 | 2.066 | −13.918 | 1.00 | 48.23 | 6 | C |
| ATOM | 2153 | CB | SER | B | 65 | −4.162 | 2.023 | −14.277 | 1.00 | 48.08 | 6 | C |
| ATOM | 2154 | OG | SER | B | 65 | −4.788 | 0.876 | −13.726 | 1.00 | 47.43 | 8 | O |
| ATOM | 2155 | C | SER | B | 65 | −2.492 | 2.106 | −12.404 | 1.00 | 48.73 | 6 | C |
| ATOM | 2156 | O | SER | B | 65 | −2.846 | 3.088 | −11.749 | 1.00 | 48.86 | 8 | O |
| ATOM | 2157 | N | ASP | B | 66 | −1.946 | 1.021 | −11.864 | 1.00 | 49.45 | 7 | N |
| ATOM | 2158 | CA | ASP | B | 66 | −1.649 | 0.888 | −10.442 | 1.00 | 50.03 | 6 | C |
| ATOM | 2159 | CB | ASP | B | 66 | −0.936 | −0.444 | −10.197 | 1.00 | 50.24 | 6 | C |
| ATOM | 2160 | CG | ASP | B | 66 | −1.298 | −1.071 | −8.871 | 1.00 | 51.55 | 6 | C |
| ATOM | 2161 | OD1 | ASP | B | 66 | −1.291 | −0.358 | −7.843 | 1.00 | 53.29 | 8 | O |
| ATOM | 2162 | OD2 | ASP | B | 66 | −1.598 | −2.281 | −8.761 | 1.00 | 53.05 | 8 | O |
| ATOM | 2163 | C | ASP | B | 66 | −0.740 | 2.020 | −9.980 | 1.00 | 49.99 | 6 | C |
| ATOM | 2164 | O | ASP | B | 66 | −0.898 | 2.552 | −8.879 | 1.00 | 50.31 | 8 | O |
| ATOM | 2165 | N | LYS | B | 67 | 0.217 | 2.388 | −10.824 | 1.00 | 49.83 | 7 | N |
| ATOM | 2166 | CA | LYS | B | 67 | 1.187 | 3.408 | −10.445 | 1.00 | 49.88 | 6 | C |
| ATOM | 2167 | CB | LYS | B | 67 | 2.486 | 2.744 | −9.968 | 1.00 | 50.16 | 6 | C |
| ATOM | 2168 | CG | LYS | B | 67 | 2.292 | 1.606 | −8.970 | 1.00 | 50.67 | 6 | C |
| ATOM | 2169 | CD | LYS | B | 67 | 3.628 | 1.115 | −8.427 | 1.00 | 52.01 | 6 | C |
| ATOM | 2170 | CE | LYS | B | 67 | 3.426 | −0.029 | −7.441 | 1.00 | 52.48 | 6 | C |
| ATOM | 2171 | NZ | LYS | B | 67 | 4.654 | −0.315 | −6.657 | 1.00 | 52.33 | 7 | N |
| ATOM | 2172 | C | LYS | B | 67 | 1.521 | 4.402 | −11.555 | 1.00 | 49.63 | 6 | C |
| ATOM | 2173 | O | LYS | B | 67 | 1.918 | 5.534 | −11.273 | 1.00 | 49.74 | 8 | O |
| ATOM | 2174 | N | TYR | B | 68 | 1.367 | 3.988 | −12.811 | 1.00 | 49.08 | 7 | N |
| ATOM | 2175 | CA | TYR | B | 68 | 1.777 | 4.841 | −13.924 | 1.00 | 48.69 | 6 | C |
| ATOM | 2176 | CB | TYR | B | 68 | 2.941 | 4.188 | −14.664 | 1.00 | 48.97 | 6 | C |
| ATOM | 2177 | CG | TYR | B | 68 | 4.094 | 3.857 | −13.750 | 1.00 | 50.19 | 6 | C |
| ATOM | 2178 | CD1 | TYR | B | 68 | 4.546 | 2.553 | −13.608 | 1.00 | 51.24 | 6 | C |
| ATOM | 2179 | CE1 | TYR | B | 68 | 5.603 | 2.252 | −12.761 | 1.00 | 52.27 | 6 | C |
| ATOM | 2180 | CZ | TYR | B | 68 | 6.207 | 3.265 | −12.043 | 1.00 | 52.23 | 6 | C |
| ATOM | 2181 | OH | TYR | B | 68 | 7.254 | 2.982 | −11.197 | 1.00 | 53.59 | 8 | O |
| ATOM | 2182 | CE2 | TYR | B | 68 | 5.771 | 4.563 | −12.167 | 1.00 | 51.84 | 6 | C |
| ATOM | 2183 | CD2 | TYR | B | 68 | 4.719 | 4.852 | −13.013 | 1.00 | 51.41 | 6 | C |
| ATOM | 2184 | C | TYR | B | 68 | 0.673 | 5.200 | −14.911 | 1.00 | 48.02 | 6 | C |
| ATOM | 2185 | O | TYR | B | 68 | 0.413 | 6.376 | −15.160 | 1.00 | 48.00 | 8 | O |
| ATOM | 2186 | N | GLY | B | 69 | 0.050 | 4.178 | −15.490 | 1.00 | 47.28 | 7 | N |
| ATOM | 2187 | CA | GLY | B | 69 | −0.998 | 4.379 | −16.475 | 1.00 | 46.31 | 6 | C |
| ATOM | 2188 | C | GLY | B | 69 | −1.173 | 3.145 | −17.341 | 1.00 | 45.70 | 6 | C |
| ATOM | 2189 | O | GLY | B | 69 | −0.731 | 2.056 | −16.975 | 1.00 | 45.54 | 8 | O |
| ATOM | 2190 | N | LEU | B | 70 | −1.823 | 3.308 | −18.487 | 1.00 | 44.96 | 7 | N |
| ATOM | 2191 | CA | LEU | B | 70 | −2.035 | 2.182 | −19.389 | 1.00 | 44.33 | 6 | C |
| ATOM | 2192 | CB | LEU | B | 70 | −3.526 | 1.973 | −19.674 | 1.00 | 44.42 | 6 | C |
| ATOM | 2193 | CG | LEU | B | 70 | −3.858 | 0.689 | −20.448 | 1.00 | 44.65 | 6 | C |
| ATOM | 2194 | CD1 | LEU | B | 70 | −5.027 | −0.062 | −19.821 | 1.00 | 44.25 | 6 | C |
| ATOM | 2195 | CD2 | LEU | B | 70 | −4.105 | 0.990 | −21.921 | 1.00 | 44.67 | 6 | C |
| ATOM | 2196 | C | LEU | B | 70 | −1.257 | 2.377 | −20.679 | 1.00 | 43.72 | 6 | C |
| ATOM | 2197 | O | LEU | B | 70 | −1.379 | 3.405 | −21.336 | 1.00 | 43.83 | 8 | O |
| ATOM | 2198 | N | GLY | B | 71 | −0.443 | 1.387 | −21.022 | 1.00 | 43.34 | 7 | N |
| ATOM | 2199 | CA | GLY | B | 71 | 0.350 | 1.426 | −22.232 | 1.00 | 42.55 | 6 | C |
| ATOM | 2200 | C | GLY | B | 71 | −0.299 | 0.531 | −23.260 | 1.00 | 42.19 | 6 | C |
| ATOM | 2201 | O | GLY | B | 71 | −0.919 | −0.468 | −22.919 | 1.00 | 41.85 | 8 | O |
| ATOM | 2202 | N | TYR | B | 72 | −0.142 | 0.878 | −24.526 | 1.00 | 42.15 | 7 | N |
| ATOM | 2203 | CA | TYR | B | 72 | −0.806 | 0.131 | −25.574 | 1.00 | 42.11 | 6 | C |
| ATOM | 2204 | CB | TYR | B | 72 | −2.250 | 0.641 | −25.732 | 1.00 | 41.72 | 6 | C |
| ATOM | 2205 | CG | TYR | B | 72 | −2.334 | 2.099 | −26.140 | 1.00 | 40.71 | 6 | C |
| ATOM | 2206 | CD1 | TYR | B | 72 | −2.322 | 2.467 | −27.481 | 1.00 | 39.92 | 6 | C |
| ATOM | 2207 | CE1 | TYR | B | 72 | −2.383 | 3.792 | −27.860 | 1.00 | 38.64 | 6 | C |
| ATOM | 2208 | CZ | TYR | B | 72 | −2.457 | 4.776 | −26.895 | 1.00 | 37.83 | 6 | C |
| ATOM | 2209 | OH | TYR | B | 72 | −2.527 | 6.092 | −27.283 | 1.00 | 37.64 | 8 | O |
| ATOM | 2210 | CE2 | TYR | B | 72 | −2.473 | 4.444 | −25.562 | 1.00 | 37.39 | 6 | C |
| ATOM | 2211 | CD2 | TYR | B | 72 | −2.409 | 3.112 | −25.188 | 1.00 | 40.00 | 6 | C |
| ATOM | 2212 | C | TYR | B | 72 | −0.069 | 0.321 | −26.877 | 1.00 | 42.59 | 6 | C |
| ATOM | 2213 | O | TYR | B | 72 | 0.690 | 1.276 | −27.039 | 1.00 | 42.25 | 8 | O |
| ATOM | 2214 | N | GLN | B | 73 | −0.296 | −0.598 | −27.804 | 1.00 | 43.00 | 7 | N |
| ATOM | 2215 | CA | GLN | B | 73 | 0.271 | −0.479 | −29.130 | 1.00 | 43.94 | 6 | C |
| ATOM | 2216 | CB | GLN | B | 73 | 1.159 | −1.686 | −29.457 | 1.00 | 43.99 | 6 | C |
| ATOM | 2217 | CG | GLN | B | 73 | 1.751 | −1.635 | −30.868 | 1.00 | 44.38 | 6 | C |
| ATOM | 2218 | CD | GLN | B | 73 | 2.136 | −3.000 | −31.409 | 1.00 | 45.38 | 6 | C |
| ATOM | 2219 | OE1 | GLN | B | 73 | 1.291 | −3.892 | −31.519 | 1.00 | 45.59 | 8 | O |
| ATOM | 2220 | NE2 | GLN | B | 73 | 3.407 | −3.162 | −31.764 | 1.00 | 44.82 | 7 | N |
| ATOM | 2221 | C | GLN | B | 73 | −0.870 | −0.395 | −30.130 | 1.00 | 44.38 | 6 | C |
| ATOM | 2222 | O | GLN | B | 73 | −1.900 | −1.046 | −29.961 | 1.00 | 44.15 | 8 | O |
| ATOM | 2223 | N | LEU | B | 74 | −0.700 | 0.435 | −31.151 | 1.00 | 45.26 | 7 | N |
| ATOM | 2224 | CA | LEU | B | 74 | −1.666 | 0.500 | −32.233 | 1.00 | 46.12 | 6 | C |
| ATOM | 2225 | CB | LEU | B | 74 | −1.819 | 1.928 | −32.749 | 1.00 | 46.00 | 6 | C |
| ATOM | 2226 | CG | LEU | B | 74 | −2.463 | 2.946 | −31.806 | 1.00 | 45.43 | 6 | C |
| ATOM | 2227 | CD1 | LEU | B | 74 | −2.767 | 4.221 | −32.563 | 1.00 | 45.01 | 6 | C |
| ATOM | 2228 | CD2 | LEU | B | 74 | −3.730 | 2.382 | −31.184 | 1.00 | 45.18 | 6 | C |
| ATOM | 2229 | C | LEU | B | 74 | −1.137 | −0.407 | −33.335 | 1.00 | 47.07 | 6 | C |
| ATOM | 2230 | O | LEU | B | 74 | 0.076 | −0.536 | −33.502 | 1.00 | 47.34 | 8 | O |
| ATOM | 2231 | N | CYS | B | 75 | −2.036 | −1.048 | −34.074 | 1.00 | 47.91 | 7 | N |
| ATOM | 2232 | CA | CYS | B | 75 | −1.637 | −1.956 | −35.149 | 1.00 | 48.87 | 6 | C |
| ATOM | 2233 | CB | CYS | B | 75 | −2.858 | −2.361 | −35.967 | 1.00 | 48.61 | 6 | C |
| ATOM | 2234 | SG | CYS | B | 75 | −3.706 | −0.962 | −36.722 | 1.00 | 49.27 | 16 | S |
| ATOM | 2235 | C | CYS | B | 75 | −0.617 | −1.289 | −36.066 | 1.00 | 49.39 | 6 | C |
| ATOM | 2236 | O | CYS | B | 75 | −0.059 | −1.916 | −36.966 | 1.00 | 49.37 | 8 | O |
| ATOM | 2237 | N | ASP | B | 76 | −0.396 | −0.003 | −35.820 | 1.00 | 50.09 | 7 | N |
| ATOM | 2238 | CA | ASP | B | 76 | 0.507 | 0.828 | −36.602 | 1.00 | 50.55 | 6 | C |
| ATOM | 2239 | CB | ASP | B | 76 | 0.195 | 2.297 | −36.309 | 1.00 | 50.73 | 6 | C |
| ATOM | 2240 | CG | ASP | B | 76 | 0.719 | 3.229 | −37.378 | 1.00 | 51.81 | 6 | C |
| ATOM | 2241 | OD1 | ASP | B | 76 | 0.891 | 2.775 | −38.531 | 1.00 | 53.10 | 8 | O |
| ATOM | 2242 | OD2 | ASP | B | 76 | 0.976 | 4.430 | −37.163 | 1.00 | 51.77 | 8 | O |
| ATOM | 2243 | C | ASP | B | 76 | 1.954 | 0.560 | −36.233 | 1.00 | 50.56 | 6 | C |
| ATOM | 2244 | O | ASP | B | 76 | 2.876 | 0.983 | −36.936 | 1.00 | 50.71 | 8 | O |
| ATOM | 2245 | N | ASN | B | 77 | 2.147 | −0.147 | −35.124 | 1.00 | 50.40 | 7 | N |
| ATOM | 2246 | CA | ASN | B | 77 | 3.473 | −0.393 | −34.582 | 1.00 | 50.27 | 6 | C |
| ATOM | 2247 | CB | ASN | B | 77 | 4.505 | −0.595 | −35.690 | 1.00 | 50.38 | 6 | C |
| ATOM | 2248 | CG | ASN | B | 77 | 4.327 | −1.907 | −36.410 | 1.00 | 51.17 | 6 | C |
| ATOM | 2249 | OD1 | ASN | B | 77 | 4.226 | −2.962 | −35.783 | 1.00 | 52.10 | 8 | O |
| ATOM | 2250 | ND2 | ASN | B | 77 | 4.283 | −1.854 | −37.736 | 1.00 | 51.93 | 7 | N |
| ATOM | 2251 | C | ASN | B | 77 | 3.849 | 0.784 | −33.701 | 1.00 | 49.87 | 6 | C |
| ATOM | 2252 | O | ASN | B | 77 | 4.838 | 0.746 | −32.966 | 1.00 | 49.97 | 8 | O |
| ATOM | 2253 | N | SER | B | 78 | 3.051 | 1.843 | −33.795 | 1.00 | 49.30 | 7 | N |
| ATOM | 2254 | CA | SER | B | 78 | 3.225 | 3.000 | −32.939 | 1.00 | 48.55 | 6 | C |
| ATOM | 2255 | CB | SER | B | 78 | 2.419 | 4.187 | −33.464 | 1.00 | 48.82 | 6 | C |
| ATOM | 2256 | OG | SER | B | 78 | 1.025 | 3.928 | −33.415 | 1.00 | 49.14 | 8 | O |
| ATOM | 2257 | C | SER | B | 78 | 2.729 | 2.588 | −31.564 | 1.00 | 47.91 | 6 | C |
| ATOM | 2258 | O | SER | B | 78 | 1.936 | 1.657 | −31.445 | 1.00 | 47.73 | 8 | O |
| ATOM | 2259 | N | VAL | B | 79 | 3.205 | 3.264 | −30.526 | 1.00 | 47.11 | 7 | N |
| ATOM | 2260 | CA | VAL | B | 79 | 2.797 | 2.940 | −29.165 | 1.00 | 46.32 | 6 | C |
| ATOM | 2261 | CB | VAL | B | 79 | 3.923 | 2.246 | −28.365 | 1.00 | 46.43 | 6 | C |
| ATOM | 2262 | CG1 | VAL | B | 79 | 4.309 | 0.926 | −29.008 | 1.00 | 46.40 | 6 | C |
| ATOM | 2263 | CG2 | VAL | B | 79 | 5.137 | 3.155 | −28.243 | 1.00 | 46.59 | 6 | C |
| ATOM | 2264 | C | VAL | B | 79 | 2.361 | 4.197 | −28.440 | 1.00 | 45.82 | 6 | C |
| ATOM | 2265 | O | VAL | B | 79 | 2.638 | 5.315 | −28.885 | 1.00 | 45.91 | 8 | O |
| ATOM | 2266 | N | GLY | B | 80 | 1.674 | 4.017 | −27.323 | 1.00 | 45.07 | 7 | N |
| ATOM | 2267 | CA | GLY | B | 80 | 1.177 | 5.149 | −26.574 | 1.00 | 44.81 | 6 | C |
| ATOM | 2268 | C | GLY | B | 80 | 0.838 | 4.787 | −25.148 | 1.00 | 44.62 | 6 | C |
| ATOM | 2269 | O | GLY | B | 80 | 0.876 | 3.620 | −24.755 | 1.00 | 44.13 | 8 | O |
| ATOM | 2270 | N | VAL | B | 81 | 0.491 | 5.802 | −24.370 | 1.00 | 44.84 | 7 | N |
| ATOM | 2271 | CA | VAL | B | 81 | 0.161 | 5.594 | −22.976 | 1.00 | 45.14 | 6 | C |
| ATOM | 2272 | CB | VAL | B | 81 | 1.441 | 5.569 | −22.116 | 1.00 | 45.19 | 6 | C |
| ATOM | 2273 | CG1 | VAL | B | 81 | 2.311 | 6.762 | −22.452 | 1.00 | 45.41 | 6 | C |
| ATOM | 2274 | CG2 | VAL | B | 81 | 1.112 | 5.535 | −20.626 | 1.00 | 45.15 | 6 | C |
| ATOM | 2275 | C | VAL | B | 81 | −0.759 | 6.695 | −22.480 | 1.00 | 45.31 | 6 | C |
| ATOM | 2276 | O | VAL | B | 81 | −0.682 | 7.842 | −22.926 | 1.00 | 45.36 | 8 | O |
| ATOM | 2277 | N | LEU | B | 82 | −1.653 | 6.327 | −21.573 | 1.00 | 45.75 | 7 | N |
| ATOM | 2278 | CA | LEU | B | 82 | −2.521 | 7.290 | −20.919 | 1.00 | 46.32 | 6 | C |
| ATOM | 2279 | CB | LEU | B | 82 | −3.994 | 6.893 | −21.052 | 1.00 | 46.22 | 6 | C |
| ATOM | 2280 | CG | LEU | B | 82 | −4.986 | 7.694 | −20.203 | 1.00 | 46.72 | 6 | C |
| ATOM | 2281 | CD1 | LEU | B | 82 | −4.728 | 9.186 | −20.324 | 1.00 | 46.40 | 6 | C |
| ATOM | 2282 | CD2 | LEU | B | 82 | −6.428 | 7.359 | −20.587 | 1.00 | 47.47 | 6 | C |
| ATOM | 2283 | C | LEU | B | 82 | −2.087 | 7.267 | −19.469 | 1.00 | 46.70 | 6 | C |
| ATOM | 2284 | O | LEU | B | 82 | −2.391 | 6.323 | −18.737 | 1.00 | 46.54 | 8 | O |
| ATOM | 2285 | N | PHE | B | 83 | −1.338 | 8.290 | −19.071 | 1.00 | 47.21 | 7 | N |
| ATOM | 2286 | CA | PHE | B | 83 | −0.816 | 8.379 | −17.713 | 1.00 | 48.06 | 6 | C |
| ATOM | 2287 | CB | PHE | B | 83 | 0.311 | 9.411 | −17.644 | 1.00 | 47.70 | 6 | C |
| ATOM | 2288 | CG | PHE | B | 83 | 1.547 | 9.006 | −18.400 | 1.00 | 47.11 | 6 | C |
| ATOM | 2289 | CD1 | PHE | B | 83 | 1.882 | 9.627 | −19.590 | 1.00 | 46.02 | 6 | C |
| ATOM | 2290 | CE1 | PHE | B | 83 | 3.021 | 9.251 | −20.284 | 1.00 | 45.84 | 6 | C |
| ATOM | 2291 | CZ | PHE | B | 83 | 3.835 | 8.251 | −19.788 | 1.00 | 45.40 | 6 | C |
| ATOM | 2292 | CE2 | PHE | B | 83 | 3.511 | 7.626 | −18.603 | 1.00 | 45.30 | 6 | C |
| ATOM | 2293 | CD2 | PHE | B | 83 | 2.375 | 8.002 | −17.915 | 1.00 | 46.06 | 6 | C |
| ATOM | 2294 | C | PHE | B | 83 | −1.907 | 8.711 | −16.704 | 1.00 | 48.88 | 6 | C |
| ATOM | 2295 | O | PHE | B | 83 | −2.950 | 9.256 | −17.065 | 1.00 | 48.97 | 8 | O |
| ATOM | 2296 | N | ASN | B | 84 | −1.650 | 8.386 | −15.438 | 1.00 | 49.94 | 7 | N |
| ATOM | 2297 | CA | ASN | B | 84 | −2.614 | 8.594 | −14.361 | 1.00 | 50.92 | 6 | C |
| ATOM | 2298 | CB | ASN | B | 84 | −2.119 | 7.949 | −13.068 | 1.00 | 50.84 | 6 | C |
| ATOM | 2299 | CG | ASN | B | 84 | −2.358 | 6.457 | −13.036 | 1.00 | 51.37 | 6 | C |
| ATOM | 2300 | OD1 | ASN | B | 84 | −2.907 | 5.882 | −13.979 | 1.00 | 51.80 | 8 | O |
| ATOM | 2301 | ND2 | ASN | B | 84 | −1.947 | 5.816 | −11.946 | 1.00 | 51.25 | 7 | N |
| ATOM | 2302 | C | ASN | B | 84 | −3.010 | 10.044 | −14.095 | 1.00 | 51.66 | 6 | C |
| ATOM | 2303 | O | ASN | B | 84 | −3.951 | 10.302 | −13.348 | 1.00 | 51.79 | 8 | O |
| ATOM | 2304 | N | ASN | B | 85 | −2.290 | 10.990 | −14.688 | 1.00 | 52.45 | 7 | N |
| ATOM | 2305 | CA | ASN | B | 85 | −2.637 | 12.397 | −14.524 | 1.00 | 53.40 | 6 | C |
| ATOM | 2306 | CB | ASN | B | 85 | −1.383 | 13.253 | −14.343 | 1.00 | 53.43 | 6 | C |
| ATOM | 2307 | CG | ASN | B | 85 | −0.268 | 12.849 | −15.281 | 1.00 | 54.35 | 6 | C |
| ATOM | 2308 | OD1 | ASN | B | 85 | −0.458 | 12.781 | −16.495 | 1.00 | 54.69 | 8 | O |
| ATOM | 2309 | ND2 | ASN | B | 85 | 0.905 | 12.563 | −14.721 | 1.00 | 55.14 | 7 | N |
| ATOM | 2310 | C | ASN | B | 85 | −3.463 | 12.901 | −15.701 | 1.00 | 53.75 | 6 | C |
| ATOM | 2311 | O | ASN | B | 85 | −3.652 | 14.105 | −15.872 | 1.00 | 53.84 | 8 | O |
| ATOM | 2312 | N | SER | B | 86 | −3.938 | 11.964 | −16.517 | 1.00 | 54.18 | 7 | N |
| ATOM | 2313 | CA | SER | B | 86 | −4.770 | 12.285 | −17.672 | 1.00 | 54.59 | 6 | C |
| ATOM | 2314 | CB | SER | B | 86 | −5.810 | 13.348 | −17.308 | 1.00 | 54.75 | 6 | C |
| ATOM | 2315 | OG | SER | B | 86 | −6.539 | 12.975 | −16.148 | 1.00 | 55.35 | 8 | O |
| ATOM | 2316 | C | SER | B | 86 | −3.972 | 12.713 | −18.911 | 1.00 | 54.75 | 6 | C |
| ATOM | 2317 | O | SER | B | 86 | −4.550 | 12.927 | −19.977 | 1.00 | 54.87 | 8 | O |
| ATOM | 2318 | N | THR | B | 87 | −2.654 | 12.846 | −18.782 | 1.00 | 54.78 | 7 | N |
| ATOM | 2319 | CA | THR | B | 87 | −1.838 | 13.224 | −19.935 | 1.00 | 54.86 | 6 | C |
| ATOM | 2320 | CB | THR | B | 87 | −0.513 | 13.884 | −19.510 | 1.00 | 54.79 | 6 | C |
| ATOM | 2321 | OG1 | THR | B | 87 | 0.303 | 12.929 | −18.821 | 1.00 | 54.67 | 8 | O |
| ATOM | 2322 | CG2 | THR | B | 87 | −0.761 | 14.972 | −18.477 | 1.00 | 55.12 | 6 | C |
| ATOM | 2323 | C | THR | B | 87 | −1.548 | 12.008 | −20.803 | 1.00 | 54.96 | 6 | C |
| ATOM | 2324 | O | THR | B | 87 | −1.619 | 10.873 | −20.338 | 1.00 | 54.54 | 8 | O |
| ATOM | 2325 | N | ARG | B | 88 | −1.210 | 12.257 | −22.063 | 1.00 | 55.37 | 7 | N |
| ATOM | 2326 | CA | ARG | B | 88 | −0.937 | 11.181 | −23.003 | 1.00 | 55.98 | 6 | C |
| ATOM | 2327 | CB | ARG | B | 88 | −2.129 | 10.996 | −23.944 | 1.00 | 56.11 | 6 | C |
| ATOM | 2328 | CG | ARG | B | 88 | −3.465 | 11.001 | −23.216 | 1.00 | 56.90 | 6 | C |
| ATOM | 2329 | CD | ARG | B | 88 | −4.642 | 11.404 | −24.076 | 1.00 | 58.32 | 6 | C |
| ATOM | 2330 | NE | ARG | B | 88 | −5.564 | 12.295 | −23.375 | 1.00 | 59.85 | 7 | N |
| ATOM | 2331 | CZ | ARG | B | 88 | −6.502 | 11.895 | −22.528 | 1.00 | 60.67 | 6 | C |
| ATOM | 2332 | NH1 | ARG | B | 88 | −6.657 | 10.607 | −22.259 | 1.00 | 61.31 | 7 | N |
| ATOM | 2333 | NH2 | ARG | B | 88 | −7.288 | 12.785 | −21.944 | 1.00 | 61.21 | 7 | N |
| ATOM | 2334 | C | ARG | B | 88 | 0.338 | 11.438 | −23.798 | 1.00 | 56.15 | 6 | C |
| ATOM | 2335 | O | ARG | B | 88 | 0.687 | 12.585 | −24.083 | 1.00 | 56.09 | 8 | O |
| ATOM | 2336 | N | LEU | B | 89 | 1.030 | 10.359 | −24.146 | 1.00 | 56.45 | 7 | N |
| ATOM | 2337 | CA | LEU | B | 89 | 2.266 | 10.442 | −24.910 | 1.00 | 56.73 | 6 | C |
| ATOM | 2338 | CB | LEU | B | 89 | 3.468 | 10.266 | −23.986 | 1.00 | 56.69 | 6 | C |
| ATOM | 2339 | CG | LEU | B | 89 | 4.845 | 10.423 | −24.630 | 1.00 | 56.69 | 6 | C |
| ATOM | 2340 | CD1 | LEU | B | 89 | 4.954 | 11.755 | −25.358 | 1.00 | 56.62 | 6 | C |
| ATOM | 2341 | CD2 | LEU | B | 89 | 5.934 | 10.287 | −23.578 | 1.00 | 56.68 | 6 | C |
| ATOM | 2342 | C | LEU | B | 89 | 2.275 | 9.372 | −25.996 | 1.00 | 56.98 | 6 | C |
| ATOM | 2343 | O | LEU | B | 89 | 2.106 | 8.188 | −25.711 | 1.00 | 56.74 | 8 | O |
| ATOM | 2344 | N | ILE | B | 90 | 2.468 | 9.798 | −27.240 | 1.00 | 57.42 | 7 | N |
| ATOM | 2345 | CA | ILE | B | 90 | 2.462 | 8.887 | −28.380 | 1.00 | 58.03 | 6 | C |
| ATOM | 2346 | CB | ILE | B | 90 | 1.402 | 9.332 | −29.412 | 1.00 | 57.99 | 6 | C |
| ATOM | 2347 | CG1 | ILE | B | 90 | 0.034 | 8.739 | −29.072 | 1.00 | 58.00 | 6 | C |
| ATOM | 2348 | CD1 | ILE | B | 90 | −0.523 | 9.185 | −27.745 | 1.00 | 58.27 | 6 | C |
| ATOM | 2349 | CG2 | ILE | B | 90 | 1.803 | 8.899 | −30.809 | 1.00 | 57.79 | 6 | C |
| ATOM | 2350 | C | ILE | B | 90 | 3.825 | 8.778 | −29.064 | 1.00 | 58.55 | 6 | C |
| ATOM | 2351 | O | ILE | B | 90 | 4.454 | 9.788 | −29.377 | 1.00 | 58.58 | 8 | O |
| ATOM | 2352 | N | LEU | B | 91 | 4.268 | 7.546 | −29.298 | 1.00 | 59.14 | 7 | N |
| ATOM | 2353 | CA | LEU | B | 91 | 5.530 | 7.296 | −29.985 | 1.00 | 59.96 | 6 | C |
| ATOM | 2354 | CB | LEU | B | 91 | 6.424 | 6.379 | −29.146 | 1.00 | 59.95 | 6 | C |
| ATOM | 2355 | CG | LEU | B | 91 | 7.784 | 5.982 | −29.726 | 1.00 | 60.22 | 6 | C |
| ATOM | 2356 | CD1 | LEU | B | 91 | 8.661 | 7.203 | −29.972 | 1.00 | 60.08 | 6 | C |
| ATOM | 2357 | CD2 | LEU | B | 91 | 8.488 | 4.989 | −28.812 | 1.00 | 60.59 | 6 | C |
| ATOM | 2358 | C | LEU | B | 91 | 5.280 | 6.681 | −31.363 | 1.00 | 60.42 | 6 | C |
| ATOM | 2359 | O | LEU | B | 91 | 4.936 | 5.505 | −31.468 | 1.00 | 60.57 | 8 | O |
| ATOM | 2360 | N | TYR | B | 92 | 5.461 | 7.483 | −32.411 | 1.00 | 61.14 | 7 | N |
| ATOM | 2361 | CA | TYR | B | 92 | 5.237 | 7.050 | −33.797 | 1.00 | 61.87 | 6 | C |
| ATOM | 2362 | CB | TYR | B | 92 | 5.565 | 8.187 | −34.769 | 1.00 | 61.89 | 6 | C |
| ATOM | 2363 | CG | TYR | B | 92 | 4.556 | 9.313 | −34.755 | 1.00 | 62.35 | 6 | C |
| ATOM | 2364 | CD1 | TYR | B | 92 | 4.621 | 10.320 | −33.799 | 1.00 | 62.74 | 6 | C |
| ATOM | 2365 | CE1 | TYR | B | 92 | 3.698 | 11.351 | −33.781 | 1.00 | 63.08 | 6 | C |
| ATOM | 2366 | CZ | TYR | B | 92 | 2.694 | 11.382 | −34.728 | 1.00 | 63.28 | 6 | C |
| ATOM | 2367 | OH | TYR | B | 92 | 1.772 | 12.407 | −34.716 | 1.00 | 63.24 | 8 | O |
| ATOM | 2368 | CE2 | TYR | B | 92 | 2.611 | 10.393 | −35.689 | 1.00 | 62.82 | 6 | C |
| ATOM | 2369 | CD2 | TYR | B | 92 | 3.535 | 9.368 | −35.696 | 1.00 | 62.47 | 6 | C |
| ATOM | 2370 | C | TYR | B | 92 | 5.998 | 5.780 | −34.198 | 1.00 | 62.32 | 6 | C |
| ATOM | 2371 | O | TYR | B | 92 | 6.924 | 5.354 | −33.507 | 1.00 | 62.29 | 8 | O |
| ATOM | 2372 | N | ASN | B | 93 | 5.609 | 5.185 | −35.325 | 1.00 | 62.98 | 7 | N |
| ATOM | 2373 | CA | ASN | B | 93 | 6.243 | 3.949 | −35.790 | 1.00 | 63.67 | 6 | C |
| ATOM | 2374 | CB | ASN | B | 93 | 5.471 | 3.297 | −36.952 | 1.00 | 63.71 | 6 | C |
| ATOM | 2375 | CG | ASN | B | 93 | 5.126 | 4.275 | −38.067 | 1.00 | 63.98 | 6 | C |
| ATOM | 2376 | OD1 | ASN | B | 93 | 5.878 | 5.205 | −38.360 | 1.00 | 64.09 | 8 | O |
| ATOM | 2377 | ND2 | ASN | B | 93 | 3.982 | 4.054 | −38.705 | 1.00 | 64.30 | 7 | N |
| ATOM | 2378 | C | ASN | B | 93 | 7.736 | 4.084 | −36.106 | 1.00 | 64.13 | 6 | C |
| ATOM | 2379 | O | ASN | B | 93 | 8.316 | 3.243 | −36.790 | 1.00 | 64.16 | 8 | O |
| ATOM | 2380 | N | ASP | B | 94 | 8.342 | 5.156 | −35.604 | 1.00 | 64.70 | 7 | N |
| ATOM | 2381 | CA | ASP | B | 94 | 9.783 | 5.361 | −35.701 | 1.00 | 65.19 | 6 | C |
| ATOM | 2382 | CB | ASP | B | 94 | 10.155 | 6.394 | −36.779 | 1.00 | 65.18 | 6 | C |
| ATOM | 2383 | CG | ASP | B | 94 | 9.643 | 7.798 | −36.473 | 1.00 | 65.18 | 6 | C |
| ATOM | 2384 | OD1 | ASP | B | 94 | 9.338 | 8.103 | −35.304 | 1.00 | 65.04 | 8 | O |
| ATOM | 2385 | OD2 | ASP | B | 94 | 9.524 | 8.677 | −37.352 | 1.00 | 65.48 | 8 | O |
| ATOM | 2386 | C | ASP | B | 94 | 10.287 | 5.771 | −34.321 | 1.00 | 65.50 | 6 | C |
| ATOM | 2387 | O | ASP | B | 94 | 10.165 | 6.927 | −33.925 | 1.00 | 65.67 | 8 | O |
| ATOM | 2388 | N | GLY | B | 95 | 10.829 | 4.809 | −33.581 | 1.00 | 65.83 | 7 | N |
| ATOM | 2389 | CA | GLY | B | 95 | 11.301 | 5.041 | −32.226 | 1.00 | 66.28 | 6 | C |
| ATOM | 2390 | C | GLY | B | 95 | 11.863 | 6.420 | −31.919 | 1.00 | 66.59 | 6 | C |
| ATOM | 2391 | O | GLY | B | 95 | 12.755 | 6.552 | −31.080 | 1.00 | 66.62 | 8 | O |
| ATOM | 2392 | N | ASP | B | 96 | 11.338 | 7.450 | −32.578 | 1.00 | 66.88 | 7 | N |
| ATOM | 2393 | CA | ASP | B | 96 | 11.809 | 8.813 | −32.356 | 1.00 | 67.18 | 6 | C |
| ATOM | 2394 | CB | ASP | B | 96 | 12.768 | 9.236 | −33.474 | 1.00 | 67.27 | 6 | C |
| ATOM | 2395 | CG | ASP | B | 96 | 13.823 | 10.219 | −32.997 | 1.00 | 67.45 | 6 | C |
| ATOM | 2396 | OD1 | ASP | B | 96 | 13.594 | 10.896 | −31.971 | 1.00 | 67.40 | 8 | O |
| ATOM | 2397 | OD2 | ASP | B | 96 | 14.916 | 10.377 | −33.581 | 1.00 | 68.03 | 8 | O |
| ATOM | 2398 | C | ASP | B | 96 | 10.672 | 9.835 | −32.208 | 1.00 | 67.32 | 6 | C |
| ATOM | 2399 | O | ASP | B | 96 | 10.487 | 10.406 | −31.134 | 1.00 | 67.40 | 8 | O |
| ATOM | 2400 | N | SER | B | 97 | 9.915 | 10.061 | −33.281 | 1.00 | 67.42 | 7 | N |
| ATOM | 2401 | CA | SER | B | 97 | 8.832 | 11.053 | −33.275 | 1.00 | 67.54 | 6 | C |
| ATOM | 2402 | CB | SER | B | 97 | 8.043 | 10.995 | −34.584 | 1.00 | 67.55 | 6 | C |
| ATOM | 2403 | OG | SER | B | 97 | 8.855 | 11.364 | −35.686 | 1.00 | 67.59 | 8 | O |
| ATOM | 2404 | C | SER | B | 97 | 7.880 | 10.931 | −32.080 | 1.00 | 67.68 | 6 | C |
| ATOM | 2405 | O | SER | B | 97 | 7.582 | 9.827 | −31.622 | 1.00 | 67.61 | 8 | O |
| ATOM | 2406 | N | LEU | B | 98 | 7.400 | 12.072 | −31.586 | 1.00 | 67.81 | 7 | N |
| ATOM | 2407 | CA | LEU | B | 98 | 6.516 | 12.089 | −30.422 | 1.00 | 68.00 | 6 | C |
| ATOM | 2408 | CB | LEU | B | 98 | 7.319 | 12.318 | −29.140 | 1.00 | 67.93 | 6 | C |
| ATOM | 2409 | CG | LEU | B | 98 | 8.190 | 11.213 | −28.554 | 1.00 | 67.95 | 6 | C |
| ATOM | 2410 | CD1 | LEU | B | 98 | 9.010 | 11.789 | −27.417 | 1.00 | 67.95 | 6 | C |
| ATOM | 2411 | CD2 | LEU | B | 98 | 7.352 | 10.048 | −28.070 | 1.00 | 67.97 | 6 | C |
| ATOM | 2412 | C | LEU | B | 98 | 5.420 | 13.146 | −30.470 | 1.00 | 68.19 | 6 | C |
| ATOM | 2413 | O | LEU | B | 98 | 5.580 | 14.210 | −31.069 | 1.00 | 68.13 | 8 | O |
| ATOM | 2414 | N | GLN | B | 99 | 4.309 | 12.832 | −29.812 | 1.00 | 68.43 | 7 | N |
| ATOM | 2415 | CA | GLN | B | 99 | 3.202 | 13.759 | −29.639 | 1.00 | 68.66 | 6 | C |
| ATOM | 2416 | CB | GLN | B | 99 | 2.012 | 13.382 | −30.521 | 1.00 | 68.63 | 6 | C |
| ATOM | 2417 | CG | GLN | B | 99 | 0.804 | 14.293 | −30.335 | 1.00 | 68.59 | 6 | C |
| ATOM | 2418 | CD | GLN | B | 99 | −0.424 | 13.810 | −31.085 | 1.00 | 68.71 | 6 | C |
| ATOM | 2419 | OE1 | GLN | B | 99 | −1.170 | 12.968 | −30.587 | 1.00 | 68.60 | 8 | O |
| ATOM | 2420 | NE2 | GLN | B | 99 | −0.641 | 14.347 | −32.278 | 1.00 | 68.61 | 7 | N |
| ATOM | 2421 | C | GLN | B | 99 | 2.802 | 13.701 | −28.170 | 1.00 | 68.91 | 6 | C |
| ATOM | 2422 | O | GLN | B | 99 | 2.559 | 12.619 | −27.634 | 1.00 | 68.84 | 8 | O |
| ATOM | 2423 | N | TYR | B | 100 | 2.757 | 14.858 | −27.517 | 1.00 | 69.20 | 7 | N |
| ATOM | 2424 | CA | TYR | B | 100 | 2.380 | 14.927 | −26.109 | 1.00 | 69.56 | 6 | C |
| ATOM | 2425 | CB | TYR | B | 100 | 3.503 | 15.557 | −25.282 | 1.00 | 69.32 | 6 | C |
| ATOM | 2426 | CG | TYR | B | 100 | 3.236 | 15.589 | −23.794 | 1.00 | 68.56 | 6 | C |
| ATOM | 2427 | CD1 | TYR | B | 100 | 3.109 | 14.415 | −23.065 | 1.00 | 67.74 | 6 | C |
| ATOM | 2428 | CE1 | TYR | B | 100 | 2.868 | 14.441 | −21.703 | 1.00 | 67.47 | 6 | C |
| ATOM | 2429 | CZ | TYR | B | 100 | 2.752 | 15.655 | −21.054 | 1.00 | 67.56 | 6 | C |
| ATOM | 2430 | OH | TYR | B | 100 | 2.513 | 15.691 | −19.699 | 1.00 | 67.02 | 8 | O |
| ATOM | 2431 | CE2 | TYR | B | 100 | 2.879 | 16.834 | −21.758 | 1.00 | 67.71 | 6 | C |
| ATOM | 2432 | CD2 | TYR | B | 100 | 3.119 | 16.796 | −23.119 | 1.00 | 68.03 | 6 | C |
| ATOM | 2433 | C | TYR | B | 100 | 1.083 | 15.713 | −25.941 | 1.00 | 70.07 | 6 | C |
| ATOM | 2434 | O | TYR | B | 100 | 0.976 | 16.854 | −26.392 | 1.00 | 70.07 | 8 | O |
| ATOM | 2435 | N | ILE | B | 101 | 0.099 | 15.093 | −25.298 | 1.00 | 70.74 | 7 | N |
| ATOM | 2436 | CA | ILE | B | 101 | −1.202 | 15.720 | −25.093 | 1.00 | 71.51 | 6 | C |
| ATOM | 2437 | CB | ILE | B | 101 | −2.311 | 14.944 | −25.840 | 1.00 | 71.47 | 6 | C |
| ATOM | 2438 | CG1 | ILE | B | 101 | −1.852 | 14.522 | −27.240 | 1.00 | 71.40 | 6 | C |
| ATOM | 2439 | CD1 | ILE | B | 101 | −1.252 | 13.132 | −27.298 | 1.00 | 71.02 | 6 | C |
| ATOM | 2440 | CG2 | ILE | B | 101 | −3.583 | 15.769 | −25.915 | 1.00 | 71.48 | 6 | C |
| ATOM | 2441 | C | ILE | B | 101 | −1.551 | 15.793 | −23.613 | 1.00 | 72.16 | 6 | C |
| ATOM | 2442 | O | ILE | B | 101 | −1.736 | 14.764 | −22.966 | 1.00 | 72.17 | 8 | O |
| ATOM | 2443 | N | GLU | B | 102 | −1.650 | 17.009 | −23.083 | 1.00 | 73.07 | 7 | N |
| ATOM | 2444 | CA | GLU | B | 102 | −1.990 | 17.206 | −21.674 | 1.00 | 73.99 | 6 | C |
| ATOM | 2445 | CB | GLU | B | 102 | −1.534 | 18.587 | −21.189 | 1.00 | 73.96 | 6 | C |
| ATOM | 2446 | CG | GLU | B | 102 | −0.031 | 18.699 | −20.984 | 1.00 | 74.39 | 6 | C |
| ATOM | 2447 | CD | GLU | B | 102 | 0.396 | 20.050 | −20.444 | 1.00 | 74.91 | 6 | C |
| ATOM | 2448 | OE1 | GLU | B | 102 | 0.630 | 20.157 | −19.222 | 1.00 | 75.31 | 8 | O |
| ATOM | 2449 | OE2 | GLU | B | 102 | 0.506 | 21.005 | −21.242 | 1.00 | 74.86 | 8 | O |
| ATOM | 2450 | C | GLU | B | 102 | −3.483 | 17.004 | −21.403 | 1.00 | 74.52 | 6 | C |
| ATOM | 2451 | O | GLU | B | 102 | −4.286 | 16.922 | −22.334 | 1.00 | 74.54 | 8 | O |
| ATOM | 2452 | N | ARG | B | 103 | −3.842 | 16.923 | −20.123 | 1.00 | 75.28 | 7 | N |
| ATOM | 2453 | CA | ARG | B | 103 | −5.227 | 16.706 | −19.704 | 1.00 | 76.03 | 6 | C |
| ATOM | 2454 | CB | ARG | B | 103 | −5.381 | 16.978 | −18.206 | 1.00 | 76.14 | 6 | C |
| ATOM | 2455 | CG | ARG | B | 103 | −4.074 | 17.057 | −17.434 | 1.00 | 76.75 | 6 | C |
| ATOM | 2456 | CD | ARG | B | 103 | −4.244 | 17.442 | −15.969 | 1.00 | 77.80 | 6 | C |
| ATOM | 2457 | NE | ARG | B | 103 | −4.767 | 16.336 | −15.170 | 1.00 | 78.42 | 7 | N |
| ATOM | 2458 | CZ | ARG | B | 103 | −5.251 | 16.462 | −13.941 | 1.00 | 78.66 | 6 | C |
| ATOM | 2459 | NH1 | ARG | B | 103 | −5.289 | 17.653 | −13.357 | 1.00 | 78.73 | 7 | N |
| ATOM | 2460 | NH2 | ARG | B | 103 | −5.699 | 15.395 | −13.294 | 1.00 | 78.70 | 7 | N |
| ATOM | 2461 | C | ARG | B | 103 | −6.174 | 17.624 | −20.460 | 1.00 | 76.38 | 6 | C |
| ATOM | 2462 | O | ARG | B | 103 | −7.267 | 17.222 | −20.862 | 1.00 | 76.42 | 8 | O |
| ATOM | 2463 | N | ASP | B | 104 | −5.734 | 18.864 | −20.646 | 1.00 | 76.80 | 7 | N |
| ATOM | 2464 | CA | ASP | B | 104 | −6.528 | 19.884 | −21.315 | 1.00 | 77.26 | 6 | C |
| ATOM | 2465 | CB | ASP | B | 104 | −5.940 | 21.265 | −21.024 | 1.00 | 77.33 | 6 | C |
| ATOM | 2466 | CG | ASP | B | 104 | −5.645 | 21.468 | −19.548 | 1.00 | 77.74 | 6 | C |
| ATOM | 2467 | OD1 | ASP | B | 104 | −6.428 | 20.963 | −18.713 | 1.00 | 78.08 | 8 | O |
| ATOM | 2468 | OD2 | ASP | B | 104 | −4.656 | 22.107 | −19.126 | 1.00 | 78.02 | 8 | O |
| ATOM | 2469 | C | ASP | B | 104 | −6.639 | 19.654 | −22.821 | 1.00 | 77.45 | 6 | C |
| ATOM | 2470 | O | ASP | B | 104 | −7.158 | 20.502 | −23.548 | 1.00 | 77.53 | 8 | O |
| ATOM | 2471 | N | GLY | B | 105 | −6.149 | 18.506 | −23.284 | 1.00 | 77.62 | 7 | N |
| ATOM | 2472 | CA | GLY | B | 105 | −6.222 | 18.146 | −24.689 | 1.00 | 77.76 | 6 | C |
| ATOM | 2473 | C | GLY | B | 105 | −5.246 | 18.881 | −25.589 | 1.00 | 77.96 | 6 | C |
| ATOM | 2474 | O | GLY | B | 105 | −5.221 | 18.656 | −26.801 | 1.00 | 77.92 | 8 | O |
| ATOM | 2475 | N | THR | B | 106 | −4.438 | 19.758 | −25.001 | 1.00 | 78.10 | 7 | N |
| ATOM | 2476 | CA | THR | B | 106 | −3.467 | 20.533 | −25.766 | 1.00 | 78.26 | 6 | C |
| ATOM | 2477 | CB | THR | B | 106 | −2.848 | 21.640 | −24.890 | 1.00 | 78.26 | 6 | C |
| ATOM | 2478 | OG1 | THR | B | 106 | −3.860 | 22.589 | −24.530 | 1.00 | 78.31 | 8 | O |
| ATOM | 2479 | CG2 | THR | B | 106 | −1.861 | 22.471 | −25.697 | 1.00 | 78.25 | 6 | C |
| ATOM | 2480 | C | THR | B | 106 | −2.371 | 19.645 | −26.351 | 1.00 | 78.36 | 6 | C |
| ATOM | 2481 | O | THR | B | 106 | −1.687 | 18.921 | −25.625 | 1.00 | 78.39 | 8 | O |
| ATOM | 2482 | N | GLU | B | 107 | −2.211 | 19.710 | −27.669 | 1.00 | 78.42 | 7 | N |
| ATOM | 2483 | CA | GLU | B | 107 | −1.207 | 18.915 | −28.365 | 1.00 | 78.51 | 6 | C |
| ATOM | 2484 | CB | GLU | B | 107 | −1.568 | 18.797 | −29.847 | 1.00 | 78.54 | 6 | C |
| ATOM | 2485 | CG | GLU | B | 107 | −3.050 | 18.591 | −30.124 | 1.00 | 78.80 | 6 | C |
| ATOM | 2486 | CD | GLU | B | 107 | −3.453 | 17.130 | −30.142 | 1.00 | 79.26 | 6 | C |
| ATOM | 2487 | OE1 | GLU | B | 107 | −2.556 | 16.267 | −30.240 | 1.00 | 79.50 | 8 | O |
| ATOM | 2488 | OE2 | GLU | B | 107 | −4.667 | 16.844 | −30.065 | 1.00 | 79.53 | 8 | O |
| ATOM | 2489 | C | GLU | B | 107 | 0.180 | 19.541 | −28.226 | 1.00 | 78.51 | 6 | C |
| ATOM | 2490 | O | GLU | B | 107 | 0.318 | 20.665 | −27.748 | 1.00 | 78.62 | 8 | O |
| ATOM | 2491 | N | SER | B | 108 | 1.199 | 18.800 | −28.652 | 1.00 | 78.50 | 7 | N |
| ATOM | 2492 | CA | SER | B | 108 | 2.585 | 19.260 | −28.626 | 1.00 | 78.45 | 6 | C |
| ATOM | 2493 | CB | SER | B | 108 | 3.051 | 19.543 | −27.199 | 1.00 | 78.46 | 6 | C |
| ATOM | 2494 | OG | SER | B | 108 | 3.040 | 18.365 | −26.415 | 1.00 | 78.62 | 8 | O |
| ATOM | 2495 | C | SER | B | 108 | 3.461 | 18.193 | −29.270 | 1.00 | 78.43 | 6 | C |
| ATOM | 2496 | O | SER | B | 108 | 3.515 | 17.057 | −28.803 | 1.00 | 78.45 | 8 | O |
| ATOM | 2497 | N | TYR | B | 109 | 4.152 | 18.564 | −30.341 | 1.00 | 78.38 | 7 | N |
| ATOM | 2498 | CA | TYR | B | 109 | 4.948 | 17.608 | −31.101 | 1.00 | 78.34 | 6 | C |
| ATOM | 2499 | CB | TYR | B | 109 | 4.589 | 17.716 | −32.585 | 1.00 | 78.39 | 6 | C |
| ATOM | 2500 | CG | TYR | B | 109 | 3.099 | 17.874 | −32.805 | 1.00 | 78.59 | 6 | C |
| ATOM | 2501 | CD1 | TYR | B | 109 | 2.483 | 19.113 | −32.660 | 1.00 | 78.75 | 6 | C |
| ATOM | 2502 | CE1 | TYR | B | 109 | 1.121 | 19.262 | −32.845 | 1.00 | 78.95 | 6 | C |
| ATOM | 2503 | CZ | TYR | B | 109 | 0.353 | 18.164 | −33.175 | 1.00 | 79.00 | 6 | C |
| ATOM | 2504 | OH | TYR | B | 109 | −1.003 | 18.310 | −33.361 | 1.00 | 79.25 | 8 | O |
| ATOM | 2505 | CE2 | TYR | B | 109 | 0.938 | 16.923 | −33.319 | 1.00 | 78.96 | 6 | C |
| ATOM | 2506 | CD2 | TYR | B | 109 | 2.303 | 16.782 | −33.129 | 1.00 | 78.86 | 6 | C |
| ATOM | 2507 | C | TYR | B | 109 | 6.449 | 17.775 | −30.881 | 1.00 | 78.23 | 6 | C |
| ATOM | 2508 | O | TYR | B | 109 | 7.040 | 18.780 | −31.276 | 1.00 | 78.29 | 8 | O |
| ATOM | 2509 | N | LEU | B | 110 | 7.057 | 16.780 | −30.243 | 1.00 | 78.03 | 7 | N |
| ATOM | 2510 | CA | LEU | B | 110 | 8.485 | 16.813 | −29.947 | 1.00 | 77.83 | 6 | C |
| ATOM | 2511 | CB | LEU | B | 110 | 8.724 | 16.946 | −28.439 | 1.00 | 77.87 | 6 | C |
| ATOM | 2512 | CG | LEU | B | 110 | 8.115 | 15.884 | −27.517 | 1.00 | 77.92 | 6 | C |
| ATOM | 2513 | CD1 | LEU | B | 110 | 8.880 | 15.816 | −26.203 | 1.00 | 77.91 | 6 | C |
| ATOM | 2514 | CD2 | LEU | B | 110 | 6.636 | 16.148 | −27.269 | 1.00 | 77.94 | 6 | C |
| ATOM | 2515 | C | LEU | B | 110 | 9.209 | 15.583 | −30.490 | 1.00 | 77.67 | 6 | C |
| ATOM | 2516 | O | LEU | B | 110 | 8.751 | 14.952 | −31.443 | 1.00 | 77.63 | 8 | O |
| ATOM | 2517 | N | THR | B | 111 | 10.342 | 15.251 | −29.877 | 1.00 | 77.46 | 7 | N |
| ATOM | 2518 | CA | THR | B | 111 | 11.153 | 14.119 | −30.311 | 1.00 | 77.27 | 6 | C |
| ATOM | 2519 | CB | THR | B | 111 | 12.266 | 14.600 | −31.267 | 1.00 | 77.30 | 6 | C |
| ATOM | 2520 | OG1 | THR | B | 111 | 11.832 | 15.769 | −31.973 | 1.00 | 77.54 | 8 | O |
| ATOM | 2521 | CG2 | THR | B | 111 | 12.498 | 13.589 | −32.373 | 1.00 | 77.32 | 6 | C |
| ATOM | 2522 | C | THR | B | 111 | 11.787 | 13.416 | −29.115 | 1.00 | 77.06 | 6 | C |
| ATOM | 2523 | O | THR | B | 111 | 11.875 | 13.985 | −28.027 | 1.00 | 77.07 | 8 | O |
| ATOM | 2524 | N | VAL | B | 112 | 12.218 | 12.174 | −29.313 | 1.00 | 76.82 | 7 | N |
| ATOM | 2525 | CA | VAL | B | 112 | 12.906 | 11.438 | −28.260 | 1.00 | 76.62 | 6 | C |
| ATOM | 2526 | CB | VAL | B | 112 | 12.893 | 9.915 | −28.505 | 1.00 | 76.66 | 6 | C |
| ATOM | 2527 | CG1 | VAL | B | 112 | 13.780 | 9.202 | −27.498 | 1.00 | 76.61 | 6 | C |
| ATOM | 2528 | CG2 | VAL | B | 112 | 11.473 | 9.370 | −28.433 | 1.00 | 76.72 | 6 | C |
| ATOM | 2529 | C | VAL | B | 112 | 14.341 | 11.943 | −28.224 | 1.00 | 76.45 | 6 | C |
| ATOM | 2530 | O | VAL | B | 112 | 15.005 | 11.911 | −27.186 | 1.00 | 76.43 | 8 | O |
| ATOM | 2531 | N | SER | B | 113 | 14.806 | 12.419 | −29.375 | 1.00 | 76.23 | 7 | N |
| ATOM | 2532 | CA | SER | B | 113 | 16.147 | 12.972 | −29.501 | 1.00 | 76.03 | 6 | C |
| ATOM | 2533 | CB | SER | B | 113 | 16.628 | 12.901 | −30.953 | 1.00 | 76.07 | 6 | C |
| ATOM | 2534 | OG | SER | B | 113 | 15.670 | 13.448 | −31.844 | 1.00 | 76.02 | 8 | O |
| ATOM | 2535 | C | SER | B | 113 | 16.169 | 14.411 | −28.996 | 1.00 | 75.80 | 6 | C |
| ATOM | 2536 | O | SER | B | 113 | 17.219 | 15.051 | −28.953 | 1.00 | 75.73 | 8 | O |
| ATOM | 2537 | N | SER | B | 114 | 14.996 | 14.911 | −28.616 | 1.00 | 75.52 | 7 | N |
| ATOM | 2538 | CA | SER | B | 114 | 14.871 | 16.254 | −28.068 | 1.00 | 75.28 | 6 | C |
| ATOM | 2539 | CB | SER | B | 114 | 13.576 | 16.910 | −28.540 | 1.00 | 75.35 | 6 | C |
| ATOM | 2540 | OG | SER | B | 114 | 12.455 | 16.348 | −27.882 | 1.00 | 75.61 | 8 | O |
| ATOM | 2541 | C | SER | B | 114 | 14.879 | 16.160 | −26.551 | 1.00 | 75.02 | 6 | C |
| ATOM | 2542 | O | SER | B | 114 | 14.202 | 16.931 | −25.869 | 1.00 | 74.97 | 8 | O |
| ATOM | 2543 | N | HIS | B | 115 | 15.647 | 15.197 | −26.046 | 1.00 | 74.69 | 7 | N |
| ATOM | 2544 | CA | HIS | B | 115 | 15.804 | 14.914 | −24.617 | 1.00 | 74.31 | 6 | C |
| ATOM | 2545 | CB | HIS | B | 115 | 17.292 | 14.925 | −24.250 | 1.00 | 74.43 | 6 | C |
| ATOM | 2546 | CG | HIS | B | 115 | 17.641 | 14.033 | −23.099 | 1.00 | 74.97 | 6 | C |
| ATOM | 2547 | ND1 | HIS | B | 115 | 18.750 | 13.214 | −23.104 | 1.00 | 75.40 | 7 | N |
| ATOM | 2548 | CE1 | HIS | B | 115 | 18.808 | 12.547 | −21.965 | 1.00 | 75.64 | 6 | C |
| ATOM | 2549 | NE2 | HIS | B | 115 | 17.776 | 12.904 | −21.221 | 1.00 | 75.94 | 7 | N |
| ATOM | 2550 | CD2 | HIS | B | 115 | 17.030 | 13.833 | −21.907 | 1.00 | 75.53 | 6 | C |
| ATOM | 2551 | C | HIS | B | 115 | 15.029 | 15.834 | −23.669 | 1.00 | 73.80 | 6 | C |
| ATOM | 2552 | O | HIS | B | 115 | 15.630 | 16.638 | −22.954 | 1.00 | 73.83 | 8 | O |
| ATOM | 2553 | N | PRO | B | 116 | 13.703 | 15.710 | −23.656 | 1.00 | 73.26 | 7 | N |
| ATOM | 2554 | CA | PRO | B | 116 | 12.855 | 16.537 | −22.789 | 1.00 | 72.72 | 6 | C |
| ATOM | 2555 | CB | PRO | B | 116 | 11.443 | 16.154 | −23.236 | 1.00 | 72.81 | 6 | C |
| ATOM | 2556 | CG | PRO | B | 116 | 11.605 | 14.775 | −23.740 | 1.00 | 73.08 | 6 | C |
| ATOM | 2557 | CD | PRO | B | 116 | 12.900 | 14.797 | −24.492 | 1.00 | 73.17 | 6 | C |
| ATOM | 2558 | C | PRO | B | 116 | 13.037 | 16.192 | −21.317 | 1.00 | 72.09 | 6 | C |
| ATOM | 2559 | O | PRO | B | 116 | 12.667 | 15.096 | −20.904 | 1.00 | 72.06 | 8 | O |
| ATOM | 2560 | N | ASN | B | 117 | 13.598 | 17.114 | −20.540 | 1.00 | 71.24 | 7 | N |
| ATOM | 2561 | CA | ASN | B | 117 | 13.809 | 16.872 | −19.119 | 1.00 | 70.39 | 6 | C |
| ATOM | 2562 | CB | ASN | B | 117 | 14.694 | 17.958 | −18.509 | 1.00 | 70.52 | 6 | C |
| ATOM | 2563 | CG | ASN | B | 117 | 16.127 | 17.877 | −18.994 | 1.00 | 70.84 | 6 | C |
| ATOM | 2564 | OD1 | ASN | B | 117 | 16.923 | 17.091 | −18.480 | 1.00 | 71.28 | 8 | O |
| ATOM | 2565 | ND2 | ASN | B | 117 | 16.462 | 18.685 | −19.994 | 1.00 | 70.97 | 7 | N |
| ATOM | 2566 | C | ASN | B | 117 | 12.489 | 16.767 | −18.367 | 1.00 | 69.59 | 6 | C |
| ATOM | 2567 | O | ASN | B | 117 | 12.315 | 15.894 | −17.516 | 1.00 | 69.52 | 8 | O |
| ATOM | 2568 | N | ALA | B | 118 | 11.560 | 17.660 | −18.691 | 1.00 | 68.62 | 7 | N |
| ATOM | 2569 | CA | ALA | B | 118 | 10.242 | 17.645 | −18.073 | 1.00 | 67.70 | 6 | C |
| ATOM | 2570 | CB | ALA | B | 118 | 9.438 | 18.858 | −18.511 | 1.00 | 67.77 | 6 | C |
| ATOM | 2571 | C | ALA | B | 118 | 9.509 | 16.354 | −18.434 | 1.00 | 66.98 | 6 | C |
| ATOM | 2572 | O | ALA | B | 118 | 8.786 | 15.786 | −17.614 | 1.00 | 66.84 | 8 | O |
| ATOM | 2573 | N | LEU | B | 119 | 9.715 | 15.890 | −19.664 | 1.00 | 66.05 | 7 | N |
| ATOM | 2574 | CA | LEU | B | 119 | 9.073 | 14.670 | −20.148 | 1.00 | 65.14 | 6 | C |
| ATOM | 2575 | CB | LEU | B | 119 | 8.456 | 14.902 | −21.531 | 1.00 | 65.24 | 6 | C |
| ATOM | 2576 | CG | LEU | B | 119 | 7.357 | 15.965 | −21.601 | 1.00 | 65.57 | 6 | C |
| ATOM | 2577 | CD1 | LEU | B | 119 | 6.827 | 16.114 | −23.020 | 1.00 | 65.85 | 6 | C |
| ATOM | 2578 | CD2 | LEU | B | 119 | 6.231 | 15.626 | −20.636 | 1.00 | 65.80 | 6 | C |
| ATOM | 2579 | C | LEU | B | 119 | 10.033 | 13.486 | −20.204 | 1.00 | 64.34 | 6 | C |
| ATOM | 2580 | O | LEU | B | 119 | 9.884 | 12.604 | −21.046 | 1.00 | 64.28 | 8 | O |
| ATOM | 2581 | N | MET | B | 120 | 11.013 | 13.466 | −19.305 | 1.00 | 63.32 | 7 | N |
| ATOM | 2582 | CA | MET | B | 120 | 11.990 | 12.378 | −19.269 | 1.00 | 62.27 | 6 | C |
| ATOM | 2583 | CB | MET | B | 120 | 13.271 | 12.809 | −18.551 | 1.00 | 62.47 | 6 | C |
| ATOM | 2584 | CG | MET | B | 120 | 14.453 | 13.018 | −19.473 | 1.00 | 63.24 | 6 | C |
| ATOM | 2585 | SD | MET | B | 120 | 14.822 | 11.543 | −20.454 | 1.00 | 65.13 | 16 | S |
| ATOM | 2586 | CE | MET | B | 120 | 15.182 | 10.353 | −19.163 | 1.00 | 65.06 | 6 | C |
| ATOM | 2587 | C | MET | B | 120 | 11.445 | 11.118 | −18.616 | 1.00 | 61.30 | 6 | C |
| ATOM | 2588 | O | MET | B | 120 | 11.685 | 10.011 | −19.096 | 1.00 | 61.03 | 8 | O |
| ATOM | 2589 | N | LYS | B | 121 | 10.728 | 11.288 | −17.512 | 1.00 | 60.15 | 7 | N |
| ATOM | 2590 | CA | LYS | B | 121 | 10.168 | 10.152 | −16.795 | 1.00 | 59.20 | 6 | C |
| ATOM | 2591 | CB | LYS | B | 121 | 9.682 | 10.571 | −15.403 | 1.00 | 59.38 | 6 | C |
| ATOM | 2592 | CG | LYS | B | 121 | 10.772 | 11.136 | −14.489 | 1.00 | 59.63 | 6 | C |
| ATOM | 2593 | CD | LYS | B | 121 | 10.256 | 11.300 | −13.062 | 1.00 | 60.08 | 6 | C |
| ATOM | 2594 | CE | LYS | B | 121 | 11.338 | 11.818 | −12.119 | 1.00 | 60.74 | 6 | C |
| ATOM | 2595 | NZ | LYS | B | 121 | 11.682 | 13.251 | −12.368 | 1.00 | 60.83 | 7 | N |
| ATOM | 2596 | C | LYS | B | 121 | 9.026 | 9.523 | −17.588 | 1.00 | 58.39 | 6 | C |
| ATOM | 2597 | O | LYS | B | 121 | 8.823 | 8.310 | −17.536 | 1.00 | 58.28 | 8 | O |
| ATOM | 2598 | N | LYS | B | 122 | 8.285 | 10.348 | −18.324 | 1.00 | 57.44 | 7 | N |
| ATOM | 2599 | CA | LYS | B | 122 | 7.166 | 9.852 | −19.125 | 1.00 | 56.58 | 6 | C |
| ATOM | 2600 | CB | LYS | B | 122 | 6.204 | 10.987 | −19.499 | 1.00 | 56.59 | 6 | C |
| ATOM | 2601 | CG | LYS | B | 122 | 5.258 | 11.375 | −18.362 | 1.00 | 56.35 | 6 | C |
| ATOM | 2602 | CD | LYS | B | 122 | 4.421 | 12.604 | −18.684 | 1.00 | 56.34 | 6 | C |
| ATOM | 2603 | CE | LYS | B | 122 | 3.443 | 12.907 | −17.547 | 1.00 | 56.68 | 6 | C |
| ATOM | 2604 | NZ | LYS | B | 122 | 2.607 | 14.115 | −17.796 | 1.00 | 55.88 | 7 | N |
| ATOM | 2605 | C | LYS | B | 122 | 7.655 | 9.097 | −20.363 | 1.00 | 56.03 | 6 | C |
| ATOM | 2606 | O | LYS | B | 122 | 7.179 | 8.000 | −20.660 | 1.00 | 55.84 | 8 | O |
| ATOM | 2607 | N | ILE | B | 123 | 8.613 | 9.686 | −21.074 | 1.00 | 55.27 | 7 | N |
| ATOM | 2608 | CA | ILE | B | 123 | 9.200 | 9.045 | −22.247 | 1.00 | 54.71 | 6 | C |
| ATOM | 2609 | CB | ILE | B | 123 | 10.249 | 9.962 | −22.902 | 1.00 | 54.74 | 6 | C |
| ATOM | 2610 | CG1 | ILE | B | 123 | 9.563 | 11.042 | −23.736 | 1.00 | 55.03 | 6 | C |
| ATOM | 2611 | CD1 | ILE | B | 123 | 10.521 | 11.855 | −24.568 | 1.00 | 55.34 | 6 | C |
| ATOM | 2612 | CG2 | ILE | B | 123 | 11.199 | 9.156 | −23.773 | 1.00 | 54.92 | 6 | C |
| ATOM | 2613 | C | ILE | B | 123 | 9.837 | 7.718 | −21.863 | 1.00 | 54.03 | 6 | C |
| ATOM | 2614 | O | ILE | B | 123 | 9.676 | 6.712 | −22.555 | 1.00 | 54.00 | 8 | O |
| ATOM | 2615 | N | THR | B | 124 | 10.560 | 7.721 | −20.750 | 1.00 | 53.37 | 7 | N |
| ATOM | 2616 | CA | THR | B | 124 | 11.200 | 6.510 | −20.255 | 1.00 | 52.73 | 6 | C |
| ATOM | 2617 | CB | THR | B | 124 | 12.011 | 6.810 | −18.976 | 1.00 | 52.82 | 6 | C |
| ATOM | 2618 | OG1 | THR | B | 124 | 13.171 | 7.577 | −19.317 | 1.00 | 51.93 | 8 | O |
| ATOM | 2619 | CG2 | THR | B | 124 | 12.587 | 5.531 | −18.391 | 1.00 | 52.26 | 6 | C |
| ATOM | 2620 | C | THR | B | 124 | 10.154 | 5.439 | −19.991 | 1.00 | 52.58 | 6 | C |
| ATOM | 2621 | O | THR | B | 124 | 10.320 | 4.285 | −20.388 | 1.00 | 52.40 | 8 | O |
| ATOM | 2622 | N | LEU | B | 125 | 9.072 | 5.827 | −19.324 | 1.00 | 52.52 | 7 | N |
| ATOM | 2623 | CA | LEU | B | 125 | 7.981 | 4.900 | −19.051 | 1.00 | 52.53 | 6 | C |
| ATOM | 2624 | CB | LEU | B | 125 | 6.868 | 5.589 | −18.264 | 1.00 | 52.34 | 6 | C |
| ATOM | 2625 | CG | LEU | B | 125 | 6.919 | 5.415 | −16.746 | 1.00 | 52.83 | 6 | C |
| ATOM | 2626 | CD1 | LEU | B | 125 | 5.986 | 6.401 | −16.059 | 1.00 | 52.57 | 6 | C |
| ATOM | 2627 | CD2 | LEU | B | 125 | 6.568 | 3.981 | −16.361 | 1.00 | 52.79 | 6 | C |
| ATOM | 2628 | C | LEU | B | 125 | 7.433 | 4.328 | −20.351 | 1.00 | 52.47 | 6 | C |
| ATOM | 2629 | O | LEU | B | 125 | 7.090 | 3.149 | −20.422 | 1.00 | 52.35 | 8 | O |
| ATOM | 2630 | N | LEU | B | 126 | 7.369 | 5.164 | −21.382 | 1.00 | 52.61 | 7 | N |
| ATOM | 2631 | CA | LEU | B | 126 | 6.852 | 4.735 | −22.678 | 1.00 | 52.77 | 6 | C |
| ATOM | 2632 | CB | LEU | B | 126 | 6.673 | 5.927 | −23.617 | 1.00 | 52.93 | 6 | C |
| ATOM | 2633 | CG | LEU | B | 126 | 5.952 | 5.609 | −24.928 | 1.00 | 53.38 | 6 | C |
| ATOM | 2634 | CD1 | LEU | B | 126 | 4.676 | 4.832 | −24.644 | 1.00 | 54.11 | 6 | C |
| ATOM | 2635 | CD2 | LEU | B | 126 | 5.642 | 6.875 | −25.714 | 1.00 | 53.94 | 6 | C |
| ATOM | 2636 | C | LEU | B | 126 | 7.759 | 3.693 | −23.326 | 1.00 | 52.83 | 6 | C |
| ATOM | 2637 | O | LEU | B | 126 | 7.284 | 2.795 | −24.021 | 1.00 | 52.65 | 8 | O |
| ATOM | 2638 | N | LYS | B | 127 | 9.064 | 3.819 | −23.095 | 1.00 | 52.84 | 7 | N |
| ATOM | 2639 | CA | LYS | B | 127 | 10.030 | 2.872 | −23.640 | 1.00 | 52.93 | 6 | C |
| ATOM | 2640 | CB | LYS | B | 127 | 11.457 | 3.327 | −23.346 | 1.00 | 53.06 | 6 | C |
| ATOM | 2641 | CG | LYS | B | 127 | 11.788 | 4.730 | −23.807 | 1.00 | 53.98 | 6 | C |
| ATOM | 2642 | CD | LYS | B | 127 | 12.228 | 4.752 | −25.255 | 1.00 | 55.23 | 6 | C |
| ATOM | 2643 | CE | LYS | B | 127 | 13.246 | 5.862 | −25.484 | 1.00 | 55.58 | 6 | C |
| ATOM | 2644 | NZ | LYS | B | 127 | 13.877 | 5.784 | −26.833 | 1.00 | 56.15 | 7 | N |
| ATOM | 2645 | C | LYS | B | 127 | 9.815 | 1.497 | −23.024 | 1.00 | 52.76 | 6 | C |
| ATOM | 2646 | O | LYS | B | 127 | 9.772 | 0.489 | −23.732 | 1.00 | 52.74 | 8 | O |
| ATOM | 2647 | N | TYR | B | 128 | 9.693 | 1.467 | −21.701 | 1.00 | 52.52 | 7 | N |
| ATOM | 2648 | CA | TYR | B | 128 | 9.475 | 0.221 | −20.977 | 1.00 | 52.44 | 6 | C |
| ATOM | 2649 | CB | TYR | B | 128 | 9.321 | 0.493 | −19.478 | 1.00 | 52.59 | 6 | C |
| ATOM | 2650 | CG | TYR | B | 128 | 8.920 | −0.707 | −18.643 | 1.00 | 53.23 | 6 | C |
| ATOM | 2651 | CD1 | TYR | B | 128 | 7.627 | −0.829 | −18.151 | 1.00 | 54.30 | 6 | C |
| ATOM | 2652 | CE1 | TYR | B | 128 | 7.252 | −1.915 | −17.385 | 1.00 | 54.61 | 6 | C |
| ATOM | 2653 | CZ | TYR | B | 128 | 8.171 | −2.894 | −17.095 | 1.00 | 54.75 | 6 | C |
| ATOM | 2654 | OH | TYR | B | 128 | 7.788 | −3.972 | −16.329 | 1.00 | 55.15 | 8 | O |
| ATOM | 2655 | CE2 | TYR | B | 128 | 9.466 | −2.798 | −17.563 | 1.00 | 54.54 | 6 | C |
| ATOM | 2656 | CD2 | TYR | B | 128 | 9.834 | −1.706 | −18.332 | 1.00 | 54.10 | 6 | C |
| ATOM | 2657 | C | TYR | B | 128 | 8.258 | −0.515 | −21.530 | 1.00 | 52.27 | 6 | C |
| ATOM | 2658 | O | TYR | B | 128 | 8.344 | −1.694 | −21.867 | 1.00 | 52.20 | 8 | O |
| ATOM | 2659 | N | PHE | B | 129 | 7.132 | 0.188 | −21.635 | 1.00 | 51.96 | 7 | N |
| ATOM | 2660 | CA | PHE | B | 129 | 5.907 | −0.407 | −22.162 | 1.00 | 51.77 | 6 | C |
| ATOM | 2661 | CB | PHE | B | 129 | 4.800 | 0.646 | −22.261 | 1.00 | 51.70 | 6 | C |
| ATOM | 2662 | CG | PHE | B | 129 | 4.180 | 0.997 | −20.942 | 1.00 | 51.57 | 6 | C |
| ATOM | 2663 | CD1 | PHE | B | 129 | 3.995 | 2.320 | −20.577 | 1.00 | 51.51 | 6 | C |
| ATOM | 2664 | CE1 | PHE | B | 129 | 3.420 | 2.647 | −19.358 | 1.00 | 51.58 | 6 | C |
| ATOM | 2665 | CZ | PHE | B | 129 | 3.024 | 1.648 | −18.495 | 1.00 | 51.40 | 6 | C |
| ATOM | 2666 | CE2 | PHE | B | 129 | 3.203 | 0.325 | −18.850 | 1.00 | 51.91 | 6 | C |
| ATOM | 2667 | CD2 | PHE | B | 129 | 3.778 | 0.003 | −20.067 | 1.00 | 51.64 | 6 | C |
| ATOM | 2668 | C | PHE | B | 129 | 6.154 | −1.040 | −23.527 | 1.00 | 51.63 | 6 | C |
| ATOM | 2669 | O | PHE | B | 129 | 5.849 | −2.210 | −23.742 | 1.00 | 51.43 | 8 | O |
| ATOM | 2670 | N | ARG | B | 130 | 6.716 | −0.251 | −24.438 | 1.00 | 51.76 | 7 | N |
| ATOM | 2671 | CA | ARG | B | 130 | 7.029 | −0.712 | −25.782 | 1.00 | 52.09 | 6 | C |
| ATOM | 2672 | CB | ARG | B | 130 | 7.826 | 0.350 | −26.540 | 1.00 | 52.12 | 6 | C |
| ATOM | 2673 | CG | ARG | B | 130 | 8.353 | −0.128 | −27.886 | 1.00 | 52.68 | 6 | C |
| ATOM | 2674 | CD | ARG | B | 130 | 9.224 | 0.881 | −28.613 | 1.00 | 53.70 | 6 | C |
| ATOM | 2675 | NE | ARG | B | 130 | 10.443 | 1.189 | −27.868 | 1.00 | 54.71 | 7 | N |
| ATOM | 2676 | CZ | ARG | B | 130 | 11.375 | 2.032 | −28.287 | 1.00 | 55.07 | 6 | C |
| ATOM | 2677 | NH1 | ARG | B | 130 | 11.230 | 2.654 | −29.451 | 1.00 | 55.48 | 7 | N |
| ATOM | 2678 | NH2 | ARG | B | 130 | 12.454 | 2.256 | −27.547 | 1.00 | 55.58 | 7 | N |
| ATOM | 2679 | C | ARG | B | 130 | 7.811 | −2.015 | −25.750 | 1.00 | 52.16 | 6 | C |
| ATOM | 2680 | O | ARG | B | 130 | 7.408 | −3.007 | −26.359 | 1.00 | 52.02 | 8 | O |
| ATOM | 2681 | N | ASN | B | 131 | 8.933 | −2.005 | −25.035 | 1.00 | 52.22 | 7 | N |
| ATOM | 2682 | CA | ASN | B | 131 | 9.780 | −3.184 | −24.921 | 1.00 | 52.32 | 6 | C |
| ATOM | 2683 | CB | ASN | B | 131 | 11.061 | −2.854 | −24.144 | 1.00 | 52.50 | 6 | C |
| ATOM | 2684 | CG | ASN | B | 131 | 11.847 | −1.714 | −24.768 | 1.00 | 53.32 | 6 | C |
| ATOM | 2685 | OD1 | ASN | B | 131 | 11.504 | −1.220 | −25.845 | 1.00 | 54.22 | 8 | O |
| ATOM | 2686 | ND2 | ASN | B | 131 | 12.910 | −1.291 | −24.092 | 1.00 | 54.67 | 7 | N |
| ATOM | 2687 | C | ASN | B | 131 | 9.054 | −4.352 | −24.260 | 1.00 | 52.15 | 6 | C |
| ATOM | 2688 | O | ASN | B | 131 | 9.245 | −5.507 | −24.644 | 1.00 | 52.01 | 8 | O |
| ATOM | 2689 | N | TYR | B | 132 | 8.222 | −4.052 | −23.266 | 1.00 | 51.97 | 7 | N |
| ATOM | 2690 | CA | TYR | B | 132 | 7.481 | −5.100 | −22.571 | 1.00 | 52.06 | 6 | C |
| ATOM | 2691 | CB | TYR | B | 132 | 6.742 | −4.542 | −21.354 | 1.00 | 52.17 | 6 | C |
| ATOM | 2692 | CG | TYR | B | 132 | 5.904 | −5.575 | −20.632 | 1.00 | 52.63 | 6 | C |
| ATOM | 2693 | CD1 | TYR | B | 132 | 6.388 | −6.228 | −19.510 | 1.00 | 52.75 | 6 | C |
| ATOM | 2694 | CE1 | TYR | B | 132 | 5.626 | −7.173 | −18.849 | 1.00 | 53.37 | 6 | C |
| ATOM | 2695 | CZ | TYR | B | 132 | 4.363 | −7.479 | −19.313 | 1.00 | 53.58 | 6 | C |
| ATOM | 2696 | OH | TYR | B | 132 | 3.603 | −8.421 | −18.658 | 1.00 | 54.12 | 8 | O |
| ATOM | 2697 | CE2 | TYR | B | 132 | 3.859 | −6.847 | −20.427 | 1.00 | 53.55 | 6 | C |
| ATOM | 2698 | CD2 | TYR | B | 132 | 4.628 | −5.901 | −21.080 | 1.00 | 53.51 | 6 | C |
| ATOM | 2699 | C | TYR | B | 132 | 6.490 | −5.799 | −23.499 | 1.00 | 51.98 | 6 | C |
| ATOM | 2700 | O | TYR | B | 132 | 6.414 | −7.023 | −23.527 | 1.00 | 51.86 | 8 | O |
| ATOM | 2701 | N | MET | B | 133 | 5.727 | −5.015 | −24.252 | 1.00 | 52.02 | 7 | N |
| ATOM | 2702 | CA | MET | B | 133 | 4.738 | −5.578 | −25.167 | 1.00 | 52.00 | 6 | C |
| ATOM | 2703 | CB | MET | B | 133 | 3.871 | −4.468 | −25.768 | 1.00 | 51.99 | 6 | C |
| ATOM | 2704 | CG | MET | B | 133 | 3.024 | −3.728 | −24.737 | 1.00 | 51.75 | 6 | C |
| ATOM | 2705 | SD | MET | B | 133 | 2.056 | −2.335 | −25.406 | 1.00 | 50.86 | 16 | S |
| ATOM | 2706 | CE | MET | B | 133 | 3.353 | −1.216 | −25.922 | 1.00 | 51.88 | 6 | C |
| ATOM | 2707 | C | MET | B | 133 | 5.424 | −6.395 | −26.261 | 1.00 | 52.18 | 6 | C |
| ATOM | 2708 | O | MET | B | 133 | 5.016 | −7.517 | −26.563 | 1.00 | 52.10 | 8 | O |
| ATOM | 2709 | N | SER | B | 134 | 6.486 | −5.829 | −26.829 | 1.00 | 52.40 | 7 | N |
| ATOM | 2710 | CA | SER | B | 134 | 7.260 | −6.479 | −27.880 | 1.00 | 52.62 | 6 | C |
| ATOM | 2711 | CB | SER | B | 134 | 8.332 | −5.523 | −28.410 | 1.00 | 52.61 | 6 | C |
| ATOM | 2712 | OG | SER | B | 134 | 9.154 | −6.146 | −29.382 | 1.00 | 53.06 | 8 | O |
| ATOM | 2713 | C | SER | B | 134 | 7.904 | −7.782 | −27.408 | 1.00 | 52.68 | 6 | C |
| ATOM | 2714 | O | SER | B | 134 | 8.330 | −8.601 | −28.223 | 1.00 | 52.83 | 8 | O |
| ATOM | 2715 | N | GLU | B | 135 | 7.953 | −7.977 | −26.095 | 1.00 | 52.77 | 7 | N |
| ATOM | 2716 | CA | GLU | B | 135 | 8.563 | −9.171 | −25.517 | 1.00 | 52.92 | 6 | C |
| ATOM | 2717 | CB | GLU | B | 135 | 9.353 | −8.814 | −24.256 | 1.00 | 53.26 | 6 | C |
| ATOM | 2718 | CG | GLU | B | 135 | 10.839 | −8.604 | −24.488 | 1.00 | 54.61 | 6 | C |
| ATOM | 2719 | CD | GLU | B | 135 | 11.646 | −8.804 | −23.224 | 1.00 | 56.43 | 6 | C |
| ATOM | 2720 | OE1 | GLU | B | 135 | 11.356 | −8.121 | −22.219 | 1.00 | 57.82 | 8 | O |
| ATOM | 2721 | OE2 | GLU | B | 135 | 12.563 | −9.650 | −23.234 | 1.00 | 57.68 | 8 | O |
| ATOM | 2722 | C | GLU | B | 135 | 7.597 | −10.309 | −25.187 | 1.00 | 52.64 | 6 | C |
| ATOM | 2723 | O | GLU | B | 135 | 7.868 | −11.463 | −25.518 | 1.00 | 52.69 | 8 | O |
| ATOM | 2724 | N | HIS | B | 136 | 6.480 | −9.988 | −24.539 | 1.00 | 52.18 | 7 | N |
| ATOM | 2725 | CA | HIS | B | 136 | 5.550 | −11.015 | −24.066 | 1.00 | 51.81 | 6 | C |
| ATOM | 2726 | CB | HIS | B | 136 | 5.302 | −10.838 | −22.564 | 1.00 | 52.15 | 6 | C |
| ATOM | 2727 | CG | HIS | B | 136 | 6.531 | −10.962 | −21.717 | 1.00 | 52.92 | 6 | C |
| ATOM | 2728 | ND1 | HIS | B | 136 | 6.978 | −12.170 | −21.230 | 1.00 | 53.81 | 7 | N |
| ATOM | 2729 | CE1 | HIS | B | 136 | 8.069 | −11.975 | −20.509 | 1.00 | 54.28 | 6 | C |
| ATOM | 2730 | NE2 | HIS | B | 136 | 8.340 | −10.681 | −20.505 | 1.00 | 54.06 | 7 | N |
| ATOM | 2731 | CD2 | HIS | B | 136 | 7.391 | −10.025 | −21.252 | 1.00 | 53.73 | 6 | C |
| ATOM | 2732 | C | HIS | B | 136 | 4.178 | −11.053 | −24.744 | 1.00 | 51.31 | 6 | C |
| ATOM | 2733 | O | HIS | B | 136 | 3.456 | −12.044 | −24.620 | 1.00 | 51.17 | 8 | O |
| ATOM | 2734 | N | LEU | B | 137 | 3.812 | −9.988 | −25.447 | 1.00 | 50.72 | 7 | N |
| ATOM | 2735 | CA | LEU | B | 137 | 2.442 | −9.878 | −25.960 | 1.00 | 50.21 | 6 | C |
| ATOM | 2736 | CB | LEU | B | 137 | 1.858 | −8.517 | −25.569 | 1.00 | 49.98 | 6 | C |
| ATOM | 2737 | CG | LEU | B | 137 | 1.925 | −8.177 | −24.078 | 1.00 | 49.21 | 6 | C |
| ATOM | 2738 | CD1 | LEU | B | 137 | 1.125 | −6.918 | −23.785 | 1.00 | 48.39 | 6 | C |
| ATOM | 2739 | CD2 | LEU | B | 137 | 1.421 | −9.335 | −23.224 | 1.00 | 48.56 | 6 | C |
| ATOM | 2740 | C | LEU | B | 137 | 2.217 | −10.138 | −27.451 | 1.00 | 49.97 | 6 | C |
| ATOM | 2741 | O | LEU | B | 137 | 3.000 | −9.713 | −28.298 | 1.00 | 49.87 | 8 | O |
| ATOM | 2742 | N | LEU | B | 138 | 1.111 | −10.818 | −27.749 | 1.00 | 49.94 | 7 | N |
| ATOM | 2743 | CA | LEU | B | 138 | 0.698 | −11.120 | −29.120 | 1.00 | 49.71 | 6 | C |
| ATOM | 2744 | CB | LEU | B | 138 | −0.210 | −12.351 | −29.129 | 1.00 | 49.67 | 6 | C |
| ATOM | 2745 | CG | LEU | B | 138 | −0.887 | −12.698 | −30.458 | 1.00 | 49.70 | 6 | C |
| ATOM | 2746 | CD1 | LEU | B | 138 | 0.106 | −13.338 | −31.416 | 1.00 | 49.27 | 6 | C |
| ATOM | 2747 | CD2 | LEU | B | 138 | −2.073 | −13.618 | −30.222 | 1.00 | 49.98 | 6 | C |
| ATOM | 2748 | C | LEU | B | 138 | −0.035 | −9.942 | −29.776 | 1.00 | 49.63 | 6 | C |
| ATOM | 2749 | O | LEU | B | 138 | −0.911 | −9.329 | −29.172 | 1.00 | 49.34 | 8 | O |
| ATOM | 2750 | N | LYS | B | 139 | 0.329 | −9.645 | −31.018 | 1.00 | 49.67 | 7 | N |
| ATOM | 2751 | CA | LYS | B | 139 | −0.260 | −8.546 | −31.775 | 1.00 | 50.01 | 6 | C |
| ATOM | 2752 | CB | LYS | B | 139 | 0.691 | −8.140 | −32.904 | 1.00 | 49.94 | 6 | C |
| ATOM | 2753 | CG | LYS | B | 139 | 0.324 | −6.867 | −33.656 | 1.00 | 50.31 | 6 | C |
| ATOM | 2754 | CD | LYS | B | 139 | 1.516 | −6.389 | −34.478 | 1.00 | 50.49 | 6 | C |
| ATOM | 2755 | CE | LYS | B | 139 | 1.096 | −5.589 | −35.696 | 1.00 | 50.83 | 6 | C |
| ATOM | 2756 | NZ | LYS | B | 139 | 0.523 | −4.273 | −35.345 | 1.00 | 51.00 | 7 | N |
| ATOM | 2757 | C | LYS | B | 139 | −1.634 | −8.921 | −32.342 | 1.00 | 50.29 | 6 | C |
| ATOM | 2758 | O | LYS | B | 139 | −1.737 | −9.750 | −33.252 | 1.00 | 50.15 | 8 | O |
| ATOM | 2759 | N | ALA | B | 140 | −2.682 | −8.306 | −31.796 | 1.00 | 50.50 | 7 | N |
| ATOM | 2760 | CA | ALA | B | 140 | −4.054 | −8.562 | −32.234 | 1.00 | 50.83 | 6 | C |
| ATOM | 2761 | CB | ALA | B | 140 | −5.050 | −7.982 | −31.239 | 1.00 | 50.79 | 6 | C |
| ATOM | 2762 | C | ALA | B | 140 | −4.322 | −8.010 | −33.626 | 1.00 | 51.19 | 6 | C |
| ATOM | 2763 | O | ALA | B | 140 | −3.855 | −6.930 | −33.979 | 1.00 | 51.15 | 8 | O |
| ATOM | 2764 | N | GLY | B | 141 | −5.086 | −8.759 | −34.414 | 1.00 | 51.87 | 7 | N |
| ATOM | 2765 | CA | GLY | B | 141 | −5.411 | −8.348 | −35.765 | 1.00 | 52.43 | 6 | C |
| ATOM | 2766 | C | GLY | B | 141 | −4.195 | −8.378 | −36.665 | 1.00 | 53.11 | 6 | C |
| ATOM | 2767 | O | GLY | B | 141 | −4.118 | −7.639 | −37.644 | 1.00 | 52.86 | 8 | O |
| ATOM | 2768 | N | ALA | B | 142 | −3.239 | −9.236 | −36.329 | 1.00 | 53.86 | 7 | N |
| ATOM | 2769 | CA | ALA | B | 142 | −2.027 | −9.374 | −37.125 | 1.00 | 54.88 | 6 | C |
| ATOM | 2770 | CB | ALA | B | 142 | −1.108 | −10.422 | −36.516 | 1.00 | 54.80 | 6 | C |
| ATOM | 2771 | C | ALA | B | 142 | −2.354 | −9.729 | −38.575 | 1.00 | 55.58 | 6 | C |
| ATOM | 2772 | O | ALA | B | 142 | −1.614 | −9.369 | −39.490 | 1.00 | 55.55 | 8 | O |
| ATOM | 2773 | N | ASN | B | 143 | −3.472 | −10.424 | −38.774 | 1.00 | 56.60 | 7 | N |
| ATOM | 2774 | CA | ASN | B | 143 | −3.899 | −10.838 | −40.109 | 1.00 | 57.64 | 6 | C |
| ATOM | 2775 | CB | ASN | B | 143 | −4.584 | −12.208 | −40.057 | 1.00 | 57.56 | 6 | C |
| ATOM | 2776 | CG | ASN | B | 143 | −5.751 | −12.246 | −39.084 | 1.00 | 57.75 | 6 | C |
| ATOM | 2777 | OD1 | ASN | B | 143 | −5.854 | −11.411 | −38.182 | 1.00 | 57.67 | 8 | O |
| ATOM | 2778 | ND2 | ASN | B | 143 | −6.634 | −13.224 | −39.259 | 1.00 | 57.49 | 7 | N |
| ATOM | 2779 | C | ASN | B | 143 | −4.808 | −9.829 | −40.806 | 1.00 | 58.43 | 6 | C |
| ATOM | 2780 | O | ASN | B | 143 | −5.391 | −10.130 | −41.850 | 1.00 | 58.60 | 8 | O |
| ATOM | 2781 | N | ILE | B | 144 | −4.927 | −8.636 | −40.231 | 1.00 | 59.33 | 7 | N |
| ATOM | 2782 | CA | ILE | B | 144 | −5.757 | −7.587 | −40.814 | 1.00 | 60.27 | 6 | C |
| ATOM | 2783 | CB | ILE | B | 144 | −6.665 | −6.953 | −39.735 | 1.00 | 60.15 | 6 | C |
| ATOM | 2784 | CG1 | ILE | B | 144 | −7.501 | −8.025 | −39.033 | 1.00 | 60.27 | 6 | C |
| ATOM | 2785 | CD1 | ILE | B | 144 | −8.419 | −7.489 | −37.949 | 1.00 | 59.65 | 6 | C |
| ATOM | 2786 | CG2 | ILE | B | 144 | −7.560 | −5.887 | −40.344 | 1.00 | 60.18 | 6 | C |
| ATOM | 2787 | C | ILE | B | 144 | −4.896 | −6.510 | −41.466 | 1.00 | 61.06 | 6 | C |
| ATOM | 2788 | O | ILE | B | 144 | −3.826 | −6.175 | −40.960 | 1.00 | 61.18 | 8 | O |
| ATOM | 2789 | N | THR | B | 145 | −5.362 | −5.971 | −42.589 | 1.00 | 62.15 | 7 | N |
| ATOM | 2790 | CA | THR | B | 145 | −4.656 | −4.894 | −43.280 | 1.00 | 63.24 | 6 | C |
| ATOM | 2791 | CB | THR | B | 145 | −4.454 | −5.233 | −44.775 | 1.00 | 63.29 | 6 | C |
| ATOM | 2792 | OG1 | THR | B | 145 | −3.538 | −6.327 | −44.907 | 1.00 | 63.28 | 8 | O |
| ATOM | 2793 | CG2 | THR | B | 145 | −3.741 | −4.092 | −45.489 | 1.00 | 63.12 | 6 | C |
| ATOM | 2794 | C | THR | B | 145 | −5.422 | −3.578 | −43.138 | 1.00 | 64.10 | 6 | C |
| ATOM | 2795 | O | THR | B | 145 | −6.539 | −3.447 | −43.641 | 1.00 | 63.93 | 8 | O |
| ATOM | 2796 | N | PRO | B | 146 | −4.812 | −2.614 | −42.449 | 1.00 | 65.06 | 7 | N |
| ATOM | 2797 | CA | PRO | B | 146 | −5.414 | −1.293 | −42.203 | 1.00 | 65.86 | 6 | C |
| ATOM | 2798 | CB | PRO | B | 146 | −4.308 | −0.542 | −41.457 | 1.00 | 65.86 | 6 | C |
| ATOM | 2799 | CG | PRO | B | 146 | −3.478 | −1.613 | −40.847 | 1.00 | 65.50 | 6 | C |
| ATOM | 2800 | CD | PRO | B | 146 | −3.484 | −2.743 | −41.826 | 1.00 | 64.99 | 6 | C |
| ATOM | 2801 | C | PRO | B | 146 | −5.832 | −0.513 | −43.457 | 1.00 | 66.81 | 6 | C |
| ATOM | 2802 | O | PRO | B | 146 | −5.676 | −0.994 | −44.581 | 1.00 | 66.84 | 8 | O |
| ATOM | 2803 | N | ARG | B | 147 | −6.324 | 0.707 | −43.244 | 1.00 | 67.90 | 7 | N |
| ATOM | 2804 | CA | ARG | B | 147 | −6.918 | 1.522 | −44.303 | 1.00 | 68.90 | 6 | C |
| ATOM | 2805 | CB | ARG | B | 147 | −8.351 | 1.872 | −43.891 | 1.00 | 68.76 | 6 | C |
| ATOM | 2806 | CG | ARG | B | 147 | −9.312 | 2.113 | −45.034 | 1.00 | 68.48 | 6 | C |
| ATOM | 2807 | CD | ARG | B | 147 | −10.729 | 2.421 | −44.577 | 1.00 | 68.19 | 6 | C |
| ATOM | 2808 | NE | ARG | B | 147 | −10.853 | 3.743 | −43.970 | 1.00 | 67.59 | 7 | N |
| ATOM | 2809 | CZ | ARG | B | 147 | −11.582 | 4.006 | −42.891 | 1.00 | 67.44 | 6 | C |
| ATOM | 2810 | NH1 | ARG | B | 147 | −12.251 | 3.036 | −42.283 | 1.00 | 67.26 | 7 | N |
| ATOM | 2811 | NH2 | ARG | B | 147 | −11.642 | 5.240 | −42.413 | 1.00 | 67.45 | 7 | N |
| ATOM | 2812 | C | ARG | B | 147 | −6.155 | 2.809 | −44.642 | 1.00 | 69.68 | 6 | C |
| ATOM | 2813 | O | ARG | B | 147 | −4.973 | 2.944 | −44.333 | 1.00 | 69.95 | 8 | O |
| ATOM | 2814 | N | GLU | B | 148 | −6.854 | 3.750 | −45.280 | 1.00 | 70.69 | 7 | N |
| ATOM | 2815 | CA | GLU | B | 148 | −6.277 | 5.031 | −45.699 | 1.00 | 71.50 | 6 | C |
| ATOM | 2816 | CB | GLU | B | 148 | −6.978 | 5.552 | −46.957 | 1.00 | 71.67 | 6 | C |
| ATOM | 2817 | CG | GLU | B | 148 | −7.163 | 4.544 | −48.078 | 1.00 | 72.40 | 6 | C |
| ATOM | 2818 | CD | GLU | B | 148 | −8.089 | 5.069 | −49.160 | 1.00 | 73.44 | 6 | C |
| ATOM | 2819 | OE1 | GLU | B | 148 | −8.789 | 6.073 | −48.902 | 1.00 | 73.72 | 8 | O |
| ATOM | 2820 | OE2 | GLU | B | 148 | −8.116 | 4.482 | −50.265 | 1.00 | 73.75 | 8 | O |
| ATOM | 2821 | C | GLU | B | 148 | −6.388 | 6.110 | −44.624 | 1.00 | 71.87 | 6 | C |
| ATOM | 2822 | O | GLU | B | 148 | −7.396 | 6.199 | −43.923 | 1.00 | 72.05 | 8 | O |
| ATOM | 2823 | N | GLY | B | 149 | −5.359 | 6.947 | −44.523 | 1.00 | 72.24 | 7 | N |
| ATOM | 2824 | CA | GLY | B | 149 | −5.339 | 8.038 | −43.564 | 1.00 | 72.61 | 6 | C |
| ATOM | 2825 | C | GLY | B | 149 | −4.753 | 9.306 | −44.162 | 1.00 | 72.89 | 6 | C |
| ATOM | 2826 | O | GLY | B | 149 | −4.102 | 9.263 | −45.209 | 1.00 | 72.91 | 8 | O |
| ATOM | 2827 | N | ASP | B | 150 | −4.985 | 10.438 | −43.500 | 1.00 | 73.09 | 7 | N |
| ATOM | 2828 | CA | ASP | B | 150 | −4.475 | 11.726 | −43.968 | 1.00 | 73.25 | 6 | C |
| ATOM | 2829 | CB | ASP | B | 150 | −5.458 | 12.850 | −43.637 | 1.00 | 73.46 | 6 | C |
| ATOM | 2830 | CG | ASP | B | 150 | −6.855 | 12.569 | −44.151 | 1.00 | 74.14 | 6 | C |
| ATOM | 2831 | OD1 | ASP | B | 150 | −6.979 | 11.880 | −45.187 | 1.00 | 74.66 | 8 | O |
| ATOM | 2832 | OD2 | ASP | B | 150 | −7.886 | 12.992 | −43.584 | 1.00 | 75.11 | 8 | O |
| ATOM | 2833 | C | ASP | B | 150 | −3.102 | 12.022 | −43.370 | 1.00 | 73.12 | 6 | C |
| ATOM | 2834 | O | ASP | B | 150 | −2.960 | 12.868 | −42.484 | 1.00 | 73.15 | 8 | O |
| ATOM | 2835 | N | GLU | B | 151 | −2.099 | 11.315 | −43.882 | 1.00 | 72.84 | 7 | N |
| ATOM | 2836 | CA | GLU | B | 151 | −0.708 | 11.406 | −43.435 | 1.00 | 72.56 | 6 | C |
| ATOM | 2837 | CB | GLU | B | 151 | 0.229 | 11.118 | −44.613 | 1.00 | 72.70 | 6 | C |
| ATOM | 2838 | CG | GLU | B | 151 | −0.106 | 9.841 | −45.369 | 1.00 | 73.28 | 6 | C |
| ATOM | 2839 | CD | GLU | B | 151 | 0.772 | 9.637 | −46.589 | 1.00 | 74.10 | 6 | C |
| ATOM | 2840 | OE1 | GLU | B | 151 | 1.606 | 10.521 | −46.876 | 1.00 | 74.33 | 8 | O |
| ATOM | 2841 | OE2 | GLU | B | 151 | 0.627 | 8.593 | −47.262 | 1.00 | 74.53 | 8 | O |
| ATOM | 2842 | C | GLU | B | 151 | −0.274 | 12.704 | −42.744 | 1.00 | 72.11 | 6 | C |
| ATOM | 2843 | O | GLU | B | 151 | 0.538 | 12.670 | −41.817 | 1.00 | 72.16 | 8 | O |
| ATOM | 2844 | N | LEU | B | 152 | −0.805 | 13.840 | −43.189 | 1.00 | 71.49 | 7 | N |
| ATOM | 2845 | CA | LEU | B | 152 | −0.397 | 15.141 | −42.647 | 1.00 | 70.86 | 6 | C |
| ATOM | 2846 | CB | LEU | B | 152 | −0.871 | 16.279 | −43.557 | 1.00 | 71.02 | 6 | C |
| ATOM | 2847 | CG | LEU | B | 152 | −0.300 | 16.285 | −44.977 | 1.00 | 71.31 | 6 | C |
| ATOM | 2848 | CD1 | LEU | B | 152 | −0.840 | 17.469 | −45.771 | 1.00 | 71.66 | 6 | C |
| ATOM | 2849 | CD2 | LEU | B | 152 | 1.222 | 16.305 | −44.946 | 1.00 | 71.67 | 6 | C |
| ATOM | 2850 | C | LEU | B | 152 | −0.829 | 15.410 | −41.201 | 1.00 | 70.20 | 6 | C |
| ATOM | 2851 | O | LEU | B | 152 | −0.299 | 16.313 | −40.551 | 1.00 | 70.25 | 8 | O |
| ATOM | 2852 | N | ALA | B | 153 | −1.784 | 14.630 | −40.703 | 1.00 | 69.21 | 7 | N |
| ATOM | 2853 | CA | ALA | B | 153 | −2.261 | 14.796 | −39.334 | 1.00 | 68.16 | 6 | C |
| ATOM | 2854 | CB | ALA | B | 153 | −2.827 | 16.197 | −39.132 | 1.00 | 68.31 | 6 | C |
| ATOM | 2855 | C | ALA | B | 153 | −3.307 | 13.746 | −38.978 | 1.00 | 67.34 | 6 | C |
| ATOM | 2856 | O | ALA | B | 153 | −4.502 | 14.042 | −38.936 | 1.00 | 67.43 | 8 | O |
| ATOM | 2857 | N | ARG | B | 154 | −2.851 | 12.524 | −38.720 | 1.00 | 66.04 | 7 | N |
| ATOM | 2858 | CA | ARG | B | 154 | −3.748 | 11.428 | −38.365 | 1.00 | 64.75 | 6 | C |
| ATOM | 2859 | CB | ARG | B | 154 | −4.341 | 10.776 | −39.617 | 1.00 | 64.95 | 6 | C |
| ATOM | 2860 | CG | ARG | B | 154 | −5.369 | 11.623 | −40.344 | 1.00 | 65.66 | 6 | C |
| ATOM | 2861 | CD | ARG | B | 154 | −6.610 | 11.944 | −39.533 | 1.00 | 66.89 | 6 | C |
| ATOM | 2862 | NE | ARG | B | 154 | −7.517 | 12.814 | −40.275 | 1.00 | 67.71 | 7 | N |
| ATOM | 2863 | CZ | ARG | B | 154 | −8.735 | 13.142 | −39.866 | 1.00 | 68.02 | 6 | C |
| ATOM | 2864 | NH1 | ARG | B | 154 | −9.197 | 12.675 | −38.715 | 1.00 | 68.34 | 7 | N |
| ATOM | 2865 | NH2 | ARG | B | 154 | −9.493 | 13.940 | −40.607 | 1.00 | 68.13 | 7 | N |
| ATOM | 2866 | C | ARG | B | 154 | −3.048 | 10.365 | −37.528 | 1.00 | 63.44 | 6 | C |
| ATOM | 2867 | O | ARG | B | 154 | −2.103 | 9.717 | −37.983 | 1.00 | 63.52 | 8 | O |
| ATOM | 2868 | N | LEU | B | 155 | −3.532 | 10.194 | −36.305 | 1.00 | 61.63 | 7 | N |
| ATOM | 2869 | CA | LEU | B | 155 | −3.025 | 9.193 | −35.375 | 1.00 | 59.76 | 6 | C |
| ATOM | 2870 | CB | LEU | B | 155 | −1.506 | 9.246 | −35.247 | 1.00 | 59.94 | 6 | C |
| ATOM | 2871 | CG | LEU | B | 155 | −0.903 | 7.990 | −34.617 | 1.00 | 60.13 | 6 | C |
| ATOM | 2872 | CD1 | LEU | B | 155 | −0.654 | 6.935 | −35.679 | 1.00 | 60.60 | 6 | C |
| ATOM | 2873 | CD2 | LEU | B | 155 | 0.385 | 8.318 | −33.897 | 1.00 | 60.79 | 6 | C |
| ATOM | 2874 | C | LEU | B | 155 | −3.670 | 9.501 | −34.041 | 1.00 | 58.15 | 6 | C |
| ATOM | 2875 | O | LEU | B | 155 | −3.347 | 10.505 | −33.406 | 1.00 | 58.10 | 8 | O |
| ATOM | 2876 | N | PRO | B | 156 | −4.587 | 8.635 | −33.625 | 1.00 | 56.42 | 7 | N |
| ATOM | 2877 | CA | PRO | B | 156 | −5.373 | 8.850 | −32.413 | 1.00 | 54.95 | 6 | C |
| ATOM | 2878 | CB | PRO | B | 156 | −6.534 | 7.884 | −32.621 | 1.00 | 55.01 | 6 | C |
| ATOM | 2879 | CG | PRO | B | 156 | −5.843 | 6.718 | −33.220 | 1.00 | 55.56 | 6 | C |
| ATOM | 2880 | CD | PRO | B | 156 | −4.956 | 7.366 | −34.279 | 1.00 | 56.24 | 6 | C |
| ATOM | 2881 | C | PRO | B | 156 | −4.633 | 8.472 | −31.145 | 1.00 | 53.41 | 6 | C |
| ATOM | 2882 | O | PRO | B | 156 | −3.687 | 7.689 | −31.174 | 1.00 | 53.43 | 8 | O |
| ATOM | 2883 | N | TYR | B | 157 | −5.075 | 9.041 | −30.034 | 1.00 | 51.51 | 7 | N |
| ATOM | 2884 | CA | TYR | B | 157 | −4.530 | 8.697 | −28.735 | 1.00 | 49.68 | 6 | C |
| ATOM | 2885 | CB | TYR | B | 157 | −3.965 | 9.931 | −28.031 | 1.00 | 49.53 | 6 | C |
| ATOM | 2886 | CG | TYR | B | 157 | −4.937 | 11.084 | −27.898 | 1.00 | 48.93 | 6 | C |
| ATOM | 2887 | CD1 | TYR | B | 157 | −5.843 | 11.139 | −26.849 | 1.00 | 48.66 | 6 | C |
| ATOM | 2888 | CE1 | TYR | B | 157 | −6.727 | 12.196 | −26.721 | 1.00 | 47.81 | 6 | C |
| ATOM | 2889 | CZ | TYR | B | 157 | −6.708 | 13.217 | −27.646 | 1.00 | 47.98 | 6 | C |
| ATOM | 2890 | OH | TYR | B | 157 | −7.583 | 14.276 | −27.522 | 1.00 | 47.77 | 8 | O |
| ATOM | 2891 | CE2 | TYR | B | 157 | −5.814 | 13.188 | −28.693 | 1.00 | 48.01 | 6 | C |
| ATOM | 2892 | CD2 | TYR | B | 157 | −4.934 | 12.128 | −28.813 | 1.00 | 48.72 | 6 | C |
| ATOM | 2893 | C | TYR | B | 157 | −5.664 | 8.102 | −27.926 | 1.00 | 48.53 | 6 | C |
| ATOM | 2894 | O | TYR | B | 157 | −6.829 | 8.241 | −28.294 | 1.00 | 48.31 | 8 | O |
| ATOM | 2895 | N | LEU | B | 158 | −5.332 | 7.439 | −26.827 | 1.00 | 47.21 | 7 | N |
| ATOM | 2896 | CA | LEU | B | 158 | −6.353 | 6.853 | −25.975 | 1.00 | 46.08 | 6 | C |
| ATOM | 2897 | CB | LEU | B | 158 | −5.738 | 5.803 | −25.068 | 1.00 | 46.07 | 6 | C |
| ATOM | 2898 | CG | LEU | B | 158 | −6.731 | 5.066 | −24.180 | 1.00 | 45.30 | 6 | C |
| ATOM | 2899 | CD1 | LEU | B | 158 | −7.716 | 4.278 | −25.042 | 1.00 | 45.13 | 6 | C |
| ATOM | 2900 | CD2 | LEU | B | 158 | −5.978 | 4.150 | −23.247 | 1.00 | 44.78 | 6 | C |
| ATOM | 2901 | C | LEU | B | 158 | −7.028 | 7.918 | −25.122 | 1.00 | 45.64 | 6 | C |
| ATOM | 2902 | O | LEU | B | 158 | −6.416 | 8.460 | −24.201 | 1.00 | 45.58 | 8 | O |
| ATOM | 2903 | N | ARG | B | 159 | −8.288 | 8.217 | −25.422 | 1.00 | 44.66 | 7 | N |
| ATOM | 2904 | CA | ARG | B | 159 | −9.013 | 9.220 | −24.650 | 1.00 | 44.17 | 6 | C |
| ATOM | 2905 | CB | ARG | B | 159 | −10.315 | 9.610 | −25.341 | 1.00 | 44.51 | 6 | C |
| ATOM | 2906 | CG | ARG | B | 159 | −10.975 | 10.832 | −24.730 | 1.00 | 47.41 | 6 | C |
| ATOM | 2907 | CD | ARG | B | 159 | −12.466 | 10.915 | −24.992 | 1.00 | 51.06 | 6 | C |
| ATOM | 2908 | NE | ARG | B | 159 | −12.853 | 12.232 | −25.485 | 1.00 | 53.52 | 7 | N |
| ATOM | 2909 | CZ | ARG | B | 159 | −12.653 | 12.641 | −26.733 | 1.00 | 55.08 | 6 | C |
| ATOM | 2910 | NH1 | ARG | B | 159 | −12.065 | 11.835 | −27.613 | 1.00 | 55.53 | 7 | N |
| ATOM | 2911 | NH2 | ARG | B | 159 | −13.039 | 13.855 | −27.105 | 1.00 | 55.21 | 7 | N |
| ATOM | 2912 | C | ARG | B | 159 | −9.315 | 8.688 | −23.259 | 1.00 | 43.00 | 6 | C |
| ATOM | 2913 | O | ARG | B | 159 | −9.125 | 9.375 | −22.257 | 1.00 | 42.82 | 8 | O |
| ATOM | 2914 | N | THR | B | 160 | −9.799 | 7.457 | −23.203 | 1.00 | 41.62 | 7 | N |
| ATOM | 2915 | CA | THR | B | 160 | −10.102 | 6.827 | −21.929 | 1.00 | 40.46 | 6 | C |
| ATOM | 2916 | CB | THR | B | 160 | −11.342 | 7.472 | −21.271 | 1.00 | 40.57 | 6 | C |
| ATOM | 2917 | OG1 | THR | B | 160 | −11.439 | 7.048 | −19.902 | 1.00 | 40.80 | 8 | O |
| ATOM | 2918 | CG2 | THR | B | 160 | −12.633 | 6.962 | −21.911 | 1.00 | 41.26 | 6 | C |
| ATOM | 2919 | C | THR | B | 160 | −10.288 | 5.327 | −22.108 | 1.00 | 39.68 | 6 | C |
| ATOM | 2920 | O | THR | B | 160 | −10.299 | 4.818 | −23.231 | 1.00 | 38.81 | 8 | O |
| ATOM | 2921 | N | TRP | B | 161 | −10.424 | 4.625 | −20.993 | 1.00 | 38.77 | 7 | N |
| ATOM | 2922 | CA | TRP | B | 161 | −10.577 | 3.185 | −21.019 | 1.00 | 38.55 | 6 | C |
| ATOM | 2923 | CB | TRP | B | 161 | −9.227 | 2.508 | −21.276 | 1.00 | 38.41 | 6 | C |
| ATOM | 2924 | CG | TRP | B | 161 | −8.281 | 2.658 | −20.114 | 1.00 | 39.64 | 6 | C |
| ATOM | 2925 | CD1 | TRP | B | 161 | −7.445 | 3.708 | −19.870 | 1.00 | 40.03 | 6 | C |
| ATOM | 2926 | NE1 | TRP | B | 161 | −6.749 | 3.499 | −18.702 | 1.00 | 41.04 | 7 | N |
| ATOM | 2927 | CE2 | TRP | B | 161 | −7.135 | 2.300 | −18.163 | 1.00 | 40.87 | 6 | C |
| ATOM | 2928 | CD2 | TRP | B | 161 | −8.105 | 1.745 | −19.024 | 1.00 | 39.93 | 6 | C |
| ATOM | 2929 | CE3 | TRP | B | 161 | −8.657 | 0.503 | −18.694 | 1.00 | 40.64 | 6 | C |
| ATOM | 2930 | CZ3 | TRP | B | 161 | −8.240 | −0.130 | −17.537 | 1.00 | 41.98 | 6 | C |
| ATOM | 2931 | CH2 | TRP | B | 161 | −7.276 | 0.450 | −16.702 | 1.00 | 41.80 | 6 | C |
| ATOM | 2932 | CZ2 | TRP | B | 161 | −6.712 | 1.661 | −16.999 | 1.00 | 41.26 | 6 | C |
| ATOM | 2933 | C | TRP | B | 161 | −11.096 | 2.741 | −19.676 | 1.00 | 37.76 | 6 | C |
| ATOM | 2934 | O | TRP | B | 161 | −11.030 | 3.491 | −18.706 | 1.00 | 37.63 | 8 | O |
| ATOM | 2935 | N | PHE | B | 162 | −11.637 | 1.531 | −19.625 | 1.00 | 37.15 | 7 | N |
| ATOM | 2936 | CA | PHE | B | 162 | −12.058 | 0.936 | −18.359 | 1.00 | 36.85 | 6 | C |
| ATOM | 2937 | CB | PHE | B | 162 | −13.321 | 1.594 | −17.770 | 1.00 | 37.15 | 6 | C |
| ATOM | 2938 | CG | PHE | B | 162 | −14.592 | 1.319 | −18.540 | 1.00 | 36.74 | 6 | C |
| ATOM | 2939 | CD1 | PHE | B | 162 | −15.378 | 0.213 | −18.246 | 1.00 | 36.22 | 6 | C |
| ATOM | 2940 | CE1 | PHE | B | 162 | −16.549 | −0.033 | −18.944 | 1.00 | 37.13 | 6 | C |
| ATOM | 2941 | CZ | PHE | B | 162 | −16.956 | 0.835 | −19.935 | 1.00 | 35.85 | 6 | C |
| ATOM | 2942 | CE2 | PHE | B | 162 | −16.191 | 1.945 | −20.235 | 1.00 | 36.28 | 6 | C |
| ATOM | 2943 | CD2 | PHE | B | 162 | −15.011 | 2.185 | −19.533 | 1.00 | 36.01 | 6 | C |
| ATOM | 2944 | C | PHE | B | 162 | −12.215 | −0.566 | −18.510 | 1.00 | 36.88 | 6 | C |
| ATOM | 2945 | O | PHE | B | 162 | −12.373 | −1.070 | −19.617 | 1.00 | 36.24 | 8 | O |
| ATOM | 2946 | N | ARG | B | 163 | −12.133 | −1.285 | −17.398 | 1.00 | 36.54 | 7 | N |
| ATOM | 2947 | CA | ARG | B | 163 | −12.281 | −2.732 | −17.446 | 1.00 | 36.80 | 6 | C |
| ATOM | 2948 | CB | ARG | B | 163 | −11.065 | −3.427 | −16.822 | 1.00 | 36.93 | 6 | C |
| ATOM | 2949 | CG | ARG | B | 163 | −10.863 | −3.121 | −15.340 | 1.00 | 37.83 | 6 | C |
| ATOM | 2950 | CD | ARG | B | 163 | −9.774 | −3.974 | −14.676 | 1.00 | 40.09 | 6 | C |
| ATOM | 2951 | NE | ARG | B | 163 | −8.523 | −3.931 | −15.431 | 1.00 | 40.80 | 7 | N |
| ATOM | 2952 | CZ | ARG | B | 163 | −7.968 | −4.978 | −16.032 | 1.00 | 41.56 | 6 | C |
| ATOM | 2953 | NH1 | ARG | B | 163 | −8.543 | −6.173 | −15.976 | 1.00 | 41.79 | 7 | N |
| ATOM | 2954 | NH2 | ARG | B | 163 | −6.826 | −4.831 | −16.689 | 1.00 | 41.68 | 7 | N |
| ATOM | 2955 | C | ARG | B | 163 | −13.541 | −3.151 | −16.714 | 1.00 | 36.35 | 6 | C |
| ATOM | 2956 | O | ARG | B | 163 | −14.005 | −2.451 | −15.820 | 1.00 | 36.32 | 8 | O |
| ATOM | 2957 | N | THR | B | 164 | −14.121 | −4.268 | −17.141 | 1.00 | 36.11 | 7 | N |
| ATOM | 2958 | CA | THR | B | 164 | −15.242 | −4.881 | −16.440 | 1.00 | 36.21 | 6 | C |
| ATOM | 2959 | CB | THR | B | 164 | −16.510 | −4.900 | −17.302 | 1.00 | 36.10 | 6 | C |
| ATOM | 2960 | OG1 | THR | B | 164 | −16.329 | −5.800 | −18.409 | 1.00 | 36.37 | 8 | O |
| ATOM | 2961 | CG2 | THR | B | 164 | −16.727 | −3.544 | −17.965 | 1.00 | 36.23 | 6 | C |
| ATOM | 2962 | C | THR | B | 164 | −14.785 | −6.311 | −16.163 | 1.00 | 36.56 | 6 | C |
| ATOM | 2963 | O | THR | B | 164 | −13.656 | −6.665 | −16.492 | 1.00 | 36.28 | 8 | O |
| ATOM | 2964 | N | ARG | B | 165 | −15.651 | −7.130 | −15.582 | 1.00 | 36.60 | 7 | N |
| ATOM | 2965 | CA | ARG | B | 165 | −15.292 | −8.518 | −15.306 | 1.00 | 37.60 | 6 | C |
| ATOM | 2966 | CB | ARG | B | 165 | −16.314 | −9.166 | −14.370 | 1.00 | 37.85 | 6 | C |
| ATOM | 2967 | CG | ARG | B | 165 | −16.517 | −8.457 | −13.039 | 1.00 | 38.71 | 6 | C |
| ATOM | 2968 | CD | ARG | B | 165 | −17.309 | −9.283 | −12.047 | 1.00 | 40.03 | 6 | C |
| ATOM | 2969 | NE | ARG | B | 165 | −16.648 | −10.561 | −11.799 | 1.00 | 42.08 | 7 | N |
| ATOM | 2970 | CZ | ARG | B | 165 | −17.224 | −11.605 | −11.210 | 1.00 | 43.13 | 6 | C |
| ATOM | 2971 | NH1 | ARG | B | 165 | −18.485 | −11.529 | −10.799 | 1.00 | 43.55 | 7 | N |
| ATOM | 2972 | NH2 | ARG | B | 165 | −16.538 | −12.725 | −11.028 | 1.00 | 42.32 | 7 | N |
| ATOM | 2973 | C | ARG | B | 165 | −15.202 | −9.372 | −16.564 | 1.00 | 37.61 | 6 | C |
| ATOM | 2974 | O | ARG | B | 165 | −14.737 | −10.510 | −16.502 | 1.00 | 38.14 | 8 | O |
| ATOM | 2975 | N | SER | B | 166 | −15.666 | −8.843 | −17.696 | 1.00 | 37.20 | 7 | N |
| ATOM | 2976 | CA | SER | B | 166 | −15.697 | −9.618 | −18.936 | 1.00 | 36.59 | 6 | C |
| ATOM | 2977 | CB | SER | B | 166 | −17.138 | −9.809 | −19.422 | 1.00 | 36.76 | 6 | C |
| ATOM | 2978 | OG | SER | B | 166 | −17.986 | −10.287 | −18.396 | 1.00 | 37.78 | 8 | O |
| ATOM | 2979 | C | SER | B | 166 | −14.885 | −9.027 | −20.076 | 1.00 | 35.94 | 6 | C |
| ATOM | 2980 | O | SER | B | 166 | −14.591 | −9.721 | −21.036 | 1.00 | 35.60 | 8 | O |
| ATOM | 2981 | N | ALA | B | 167 | −14.525 | −7.751 | −19.986 | 1.00 | 35.33 | 7 | N |
| ATOM | 2982 | CA | ALA | B | 167 | −13.796 | −7.124 | −21.082 | 1.00 | 35.07 | 6 | C |
| ATOM | 2983 | CB | ALA | B | 167 | −14.776 | −6.801 | −22.223 | 1.00 | 35.05 | 6 | C |
| ATOM | 2984 | C | ALA | B | 167 | −13.039 | −5.862 | −20.692 | 1.00 | 34.47 | 6 | C |
| ATOM | 2985 | O | ALA | B | 167 | −13.225 | −5.328 | −19.607 | 1.00 | 34.75 | 8 | O |
| ATOM | 2986 | N | ILE | B | 168 | −12.165 | −5.411 | −21.585 | 1.00 | 34.39 | 7 | N |
| ATOM | 2987 | CA | ILE | B | 168 | −11.539 | −4.101 | −21.450 | 1.00 | 34.20 | 6 | C |
| ATOM | 2988 | CB | ILE | B | 168 | −9.998 | −4.158 | −21.534 | 1.00 | 34.23 | 6 | C |
| ATOM | 2989 | CG1 | ILE | B | 168 | −9.404 | −2.744 | −21.420 | 1.00 | 34.54 | 6 | C |
| ATOM | 2990 | CD1 | ILE | B | 168 | −7.881 | −2.715 | −21.338 | 1.00 | 36.87 | 6 | C |
| ATOM | 2991 | CG2 | ILE | B | 168 | −9.533 | −4.816 | −22.826 | 1.00 | 34.54 | 6 | C |
| ATOM | 2992 | C | ILE | B | 168 | −12.127 | −3.246 | −22.574 | 1.00 | 34.13 | 6 | C |
| ATOM | 2993 | O | ILE | B | 168 | −12.311 | −3.739 | −23.698 | 1.00 | 33.84 | 8 | O |
| ATOM | 2994 | N | ILE | B | 169 | −12.468 | −1.997 | −22.249 | 1.00 | 33.64 | 7 | N |
| ATOM | 2995 | CA | ILE | B | 169 | −13.064 | −1.057 | −23.202 | 1.00 | 33.32 | 6 | C |
| ATOM | 2996 | CB | ILE | B | 169 | −14.420 | −0.521 | −22.676 | 1.00 | 33.61 | 6 | C |
| ATOM | 2997 | CG1 | ILE | B | 169 | −15.469 | −1.635 | −22.639 | 1.00 | 33.72 | 6 | C |
| ATOM | 2998 | CD1 | ILE | B | 169 | −15.300 | −2.621 | −21.494 | 1.00 | 36.49 | 6 | C |
| ATOM | 2999 | CG2 | ILE | B | 169 | −14.922 | 0.618 | −23.555 | 1.00 | 33.32 | 6 | C |
| ATOM | 3000 | C | ILE | B | 169 | −12.099 | 0.095 | −23.474 | 1.00 | 33.28 | 6 | C |
| ATOM | 3001 | O | ILE | B | 169 | −11.649 | 0.774 | −22.552 | 1.00 | 33.30 | 8 | O |
| ATOM | 3002 | N | LEU | B | 170 | −11.765 | 0.292 | −24.740 | 1.00 | 33.25 | 7 | N |
| ATOM | 3003 | CA | LEU | B | 170 | −10.801 | 1.310 | −25.137 | 1.00 | 33.87 | 6 | C |
| ATOM | 3004 | CB | LEU | B | 170 | −9.625 | 0.642 | −25.845 | 1.00 | 33.84 | 6 | C |
| ATOM | 3005 | CG | LEU | B | 170 | −8.876 | −0.317 | −24.920 | 1.00 | 34.72 | 6 | C |
| ATOM | 3006 | CD1 | LEU | B | 170 | −8.437 | −1.581 | −25.646 | 1.00 | 36.16 | 6 | C |
| ATOM | 3007 | CD2 | LEU | B | 170 | −7.686 | 0.400 | −24.301 | 1.00 | 35.50 | 6 | C |
| ATOM | 3008 | C | LEU | B | 170 | −11.434 | 2.353 | −26.043 | 1.00 | 34.26 | 6 | C |
| ATOM | 3009 | O | LEU | B | 170 | −11.923 | 2.033 | −27.118 | 1.00 | 33.92 | 8 | O |
| ATOM | 3010 | N | HIS | B | 171 | −11.406 | 3.606 | −25.602 | 1.00 | 35.06 | 7 | N |
| ATOM | 3011 | CA | HIS | B | 171 | −11.995 | 4.704 | −26.364 | 1.00 | 35.98 | 6 | C |
| ATOM | 3012 | CB | HIS | B | 171 | −12.955 | 5.487 | −25.460 | 1.00 | 35.84 | 6 | C |
| ATOM | 3013 | CG | HIS | B | 171 | −13.669 | 6.609 | −26.147 | 1.00 | 36.49 | 6 | C |
| ATOM | 3014 | ND1 | HIS | B | 171 | −14.038 | 6.562 | −27.473 | 1.00 | 37.25 | 7 | N |
| ATOM | 3015 | CE1 | HIS | B | 171 | −14.645 | 7.690 | −27.800 | 1.00 | 37.12 | 6 | C |
| ATOM | 3016 | NE2 | HIS | B | 171 | −14.690 | 8.463 | −26.731 | 1.00 | 36.55 | 7 | N |
| ATOM | 3017 | CD2 | HIS | B | 171 | −14.087 | 7.811 | −25.683 | 1.00 | 36.20 | 6 | C |
| ATOM | 3018 | C | HIS | B | 171 | −10.918 | 5.622 | −26.955 | 1.00 | 36.33 | 6 | C |
| ATOM | 3019 | O | HIS | B | 171 | −10.269 | 6.368 | −26.227 | 1.00 | 36.67 | 8 | O |
| ATOM | 3020 | N | LEU | B | 172 | −10.742 | 5.561 | −28.274 | 1.00 | 36.92 | 7 | N |
| ATOM | 3021 | CA | LEU | B | 172 | −9.744 | 6.368 | −28.976 | 1.00 | 37.64 | 6 | C |
| ATOM | 3022 | CB | LEU | B | 172 | −9.212 | 5.612 | −30.201 | 1.00 | 37.85 | 6 | C |
| ATOM | 3023 | CG | LEU | B | 172 | −8.422 | 4.324 | −29.951 | 1.00 | 37.61 | 6 | C |
| ATOM | 3024 | CD1 | LEU | B | 172 | −8.052 | 3.648 | −31.266 | 1.00 | 38.15 | 6 | C |
| ATOM | 3025 | CD2 | LEU | B | 172 | −7.169 | 4.620 | −29.132 | 1.00 | 38.00 | 6 | C |
| ATOM | 3026 | C | LEU | B | 172 | −10.285 | 7.735 | −29.400 | 1.00 | 38.28 | 6 | C |
| ATOM | 3027 | O | LEU | B | 172 | −11.499 | 7.924 | −29.529 | 1.00 | 38.09 | 8 | O |
| ATOM | 3028 | N | SER | B | 173 | −9.372 | 8.675 | −29.642 | 1.00 | 38.69 | 7 | N |
| ATOM | 3029 | CA | SER | B | 173 | −9.727 | 10.056 | −29.982 | 1.00 | 39.00 | 6 | C |
| ATOM | 3030 | CB | SER | B | 173 | −8.496 | 10.959 | −29.858 | 1.00 | 39.22 | 6 | C |
| ATOM | 3031 | OG | SER | B | 173 | −7.450 | 10.474 | −30.683 | 1.00 | 38.70 | 8 | O |
| ATOM | 3032 | C | SER | B | 173 | −10.358 | 10.237 | −31.360 | 1.00 | 39.22 | 6 | C |
| ATOM | 3033 | O | SER | B | 173 | −10.952 | 11.283 | −31.639 | 1.00 | 39.75 | 8 | O |
| ATOM | 3034 | N | ASN | B | 174 | −10.222 | 9.241 | −32.231 | 1.00 | 38.69 | 7 | N |
| ATOM | 3035 | CA | ASN | B | 174 | −10.866 | 9.325 | −33.533 | 1.00 | 38.56 | 6 | C |
| ATOM | 3036 | CB | ASN | B | 174 | −10.104 | 8.543 | −34.607 | 1.00 | 38.62 | 6 | C |
| ATOM | 3037 | CG | ASN | B | 174 | −10.080 | 7.046 | −34.351 | 1.00 | 39.22 | 6 | C |
| ATOM | 3038 | OD1 | ASN | B | 174 | −10.567 | 6.561 | −33.322 | 1.00 | 39.24 | 8 | O |
| ATOM | 3039 | ND2 | ASN | B | 174 | −9.499 | 6.302 | −35.293 | 1.00 | 37.86 | 7 | N |
| ATOM | 3040 | C | ASN | B | 174 | −12.323 | 8.874 | −33.439 | 1.00 | 37.88 | 6 | C |
| ATOM | 3041 | O | ASN | B | 174 | −13.026 | 8.800 | −34.443 | 1.00 | 37.60 | 8 | O |
| ATOM | 3042 | N | GLY | B | 175 | −12.754 | 8.565 | −32.220 | 1.00 | 37.14 | 7 | N |
| ATOM | 3043 | CA | GLY | B | 175 | −14.126 | 8.163 | −31.968 | 1.00 | 36.74 | 6 | C |
| ATOM | 3044 | C | GLY | B | 175 | −14.361 | 6.664 | −31.880 | 1.00 | 36.07 | 6 | C |
| ATOM | 3045 | O | GLY | B | 175 | −15.415 | 6.231 | −31.431 | 1.00 | 35.97 | 8 | O |
| ATOM | 3046 | N | SER | B | 176 | −13.386 | 5.871 | −32.308 | 1.00 | 35.44 | 7 | N |
| ATOM | 3047 | CA | SER | B | 176 | −13.526 | 4.419 | −32.266 | 1.00 | 34.98 | 6 | C |
| ATOM | 3048 | CB | SER | B | 176 | −12.402 | 3.737 | −33.039 | 1.00 | 34.77 | 6 | C |
| ATOM | 3049 | OG | SER | B | 176 | −12.541 | 3.974 | −34.423 | 1.00 | 35.05 | 8 | O |
| ATOM | 3050 | C | SER | B | 176 | −13.557 | 3.885 | −30.847 | 1.00 | 34.41 | 6 | C |
| ATOM | 3051 | O | SER | B | 176 | −12.898 | 4.415 | −29.954 | 1.00 | 34.86 | 8 | O |
| ATOM | 3052 | N | VAL | B | 177 | −14.339 | 2.832 | −30.646 | 1.00 | 33.80 | 7 | N |
| ATOM | 3053 | CA | VAL | B | 177 | −14.424 | 2.178 | −29.355 | 1.00 | 32.73 | 6 | C |
| ATOM | 3054 | CB | VAL | B | 177 | −15.810 | 2.340 | −28.734 | 1.00 | 33.19 | 6 | C |
| ATOM | 3055 | CG1 | VAL | B | 177 | −15.914 | 1.543 | −27.446 | 1.00 | 32.36 | 6 | C |
| ATOM | 3056 | CG2 | VAL | B | 177 | −16.107 | 3.823 | −28.466 | 1.00 | 32.87 | 6 | C |
| ATOM | 3057 | C | VAL | B | 177 | −14.103 | 0.698 | −29.568 | 1.00 | 32.66 | 6 | C |
| ATOM | 3058 | O | VAL | B | 177 | −14.716 | 0.032 | −30.407 | 1.00 | 31.91 | 8 | O |
| ATOM | 3059 | N | GLN | B | 178 | −13.120 | 0.200 | −28.829 | 1.00 | 31.88 | 7 | N |
| ATOM | 3060 | CA | GLN | B | 178 | −12.699 | −1.185 | −28.973 | 1.00 | 32.15 | 6 | C |
| ATOM | 3061 | CB | GLN | B | 178 | −11.198 | −1.274 | −29.260 | 1.00 | 32.48 | 6 | C |
| ATOM | 3062 | CG | GLN | B | 178 | −10.692 | −2.708 | −29.355 | 1.00 | 32.29 | 6 | C |
| ATOM | 3063 | CD | GLN | B | 178 | −9.298 | −2.798 | −29.915 | 1.00 | 32.59 | 6 | C |
| ATOM | 3064 | OE1 | GLN | B | 178 | −8.862 | −1.923 | −30.671 | 1.00 | 32.25 | 8 | O |
| ATOM | 3065 | NE2 | GLN | B | 178 | −8.589 | −3.859 | −29.552 | 1.00 | 33.06 | 7 | N |
| ATOM | 3066 | C | GLN | B | 178 | −13.029 | −1.947 | −27.715 | 1.00 | 31.55 | 6 | C |
| ATOM | 3067 | O | GLN | B | 178 | −12.812 | −1.449 | −26.609 | 1.00 | 31.51 | 8 | O |
| ATOM | 3068 | N | ILE | B | 179 | −13.587 | −3.144 | −27.885 | 1.00 | 31.35 | 7 | N |
| ATOM | 3069 | CA | ILE | B | 179 | −13.932 | −3.995 | −26.755 | 1.00 | 30.76 | 6 | C |
| ATOM | 3070 | CB | ILE | B | 179 | −15.466 | −4.117 | −26.602 | 1.00 | 30.79 | 6 | C |
| ATOM | 3071 | CG1 | ILE | B | 179 | −16.121 | −2.739 | −26.491 | 1.00 | 30.81 | 6 | C |
| ATOM | 3072 | CD1 | ILE | B | 179 | −17.647 | −2.801 | −26.483 | 1.00 | 31.41 | 6 | C |
| ATOM | 3073 | CG2 | ILE | B | 179 | −15.828 | −4.966 | −25.389 | 1.00 | 30.53 | 6 | C |
| ATOM | 3074 | C | ILE | B | 179 | −13.299 | −5.378 | −26.934 | 1.00 | 31.25 | 6 | C |
| ATOM | 3075 | O | ILE | B | 179 | −13.595 | −6.082 | −27.909 | 1.00 | 30.19 | 8 | O |
| ATOM | 3076 | N | ASN | B | 180 | −12.419 | −5.750 | −26.004 | 1.00 | 31.44 | 7 | N |
| ATOM | 3077 | CA | ASN | B | 180 | −11.754 | −7.059 | −26.029 | 1.00 | 32.18 | 6 | C |
| ATOM | 3078 | CB | ASN | B | 180 | −10.245 | −6.933 | −25.787 | 1.00 | 32.08 | 6 | C |
| ATOM | 3079 | CG | ASN | B | 180 | −9.514 | −6.306 | −26.943 | 1.00 | 33.32 | 6 | C |
| ATOM | 3080 | OD1 | ASN | B | 180 | −10.120 | −5.723 | −27.832 | 1.00 | 33.17 | 8 | O |
| ATOM | 3081 | ND2 | ASN | B | 180 | −8.187 | −6.419 | −26.936 | 1.00 | 33.68 | 7 | N |
| ATOM | 3082 | C | ASN | B | 180 | −12.322 | −7.922 | −24.930 | 1.00 | 32.14 | 6 | C |
| ATOM | 3083 | O | ASN | B | 180 | −12.172 | −7.597 | −23.750 | 1.00 | 32.05 | 8 | O |
| ATOM | 3084 | N | PHE | B | 181 | −12.977 | −9.016 | −25.302 | 1.00 | 32.45 | 7 | N |
| ATOM | 3085 | CA | PHE | B | 181 | −13.565 | −9.919 | −24.318 | 1.00 | 32.92 | 6 | C |
| ATOM | 3086 | CB | PHE | B | 181 | −14.752 | −10.685 | −24.921 | 1.00 | 32.53 | 6 | C |
| ATOM | 3087 | CG | PHE | B | 181 | −15.944 | −9.809 | −25.216 | 1.00 | 33.45 | 6 | C |
| ATOM | 3088 | CD1 | PHE | B | 181 | −16.174 | −9.336 | −26.498 | 1.00 | 31.84 | 6 | C |
| ATOM | 3089 | CE1 | PHE | B | 181 | −17.263 | −8.509 | −26.770 | 1.00 | 32.71 | 6 | C |
| ATOM | 3090 | CZ | PHE | B | 181 | −18.130 | −8.158 | −25.757 | 1.00 | 31.84 | 6 | C |
| ATOM | 3091 | CE2 | PHE | B | 181 | −17.904 | −8.617 | −24.474 | 1.00 | 32.79 | 6 | C |
| ATOM | 3092 | CD2 | PHE | B | 181 | −16.815 | −9.435 | −24.204 | 1.00 | 32.67 | 6 | C |
| ATOM | 3093 | C | PHE | B | 181 | −12.505 | −10.875 | −23.766 | 1.00 | 33.64 | 6 | C |
| ATOM | 3094 | O | PHE | B | 181 | −11.829 | −11.555 | −24.525 | 1.00 | 33.85 | 8 | O |
| ATOM | 3095 | N | PHE | B | 182 | −12.376 | −10.912 | −22.442 | 1.00 | 34.71 | 7 | N |
| ATOM | 3096 | CA | PHE | B | 182 | −11.360 | −11.722 | −21.755 | 1.00 | 35.89 | 6 | C |
| ATOM | 3097 | CB | PHE | B | 182 | −11.391 | −11.442 | −20.245 | 1.00 | 35.83 | 6 | C |
| ATOM | 3098 | CG | PHE | B | 182 | −11.052 | −10.023 | −19.877 | 1.00 | 35.30 | 6 | C |
| ATOM | 3099 | CD1 | PHE | B | 182 | −11.754 | −9.373 | −18.879 | 1.00 | 34.96 | 6 | C |
| ATOM | 3100 | CE1 | PHE | B | 182 | −11.449 | −8.081 | −18.533 | 1.00 | 35.25 | 6 | C |
| ATOM | 3101 | CZ | PHE | B | 182 | −10.421 | −7.413 | −19.185 | 1.00 | 35.90 | 6 | C |
| ATOM | 3102 | CE2 | PHE | B | 182 | −9.717 | −8.045 | −20.180 | 1.00 | 35.75 | 6 | C |
| ATOM | 3103 | CD2 | PHE | B | 182 | −10.033 | −9.348 | −20.521 | 1.00 | 35.84 | 6 | C |
| ATOM | 3104 | C | PHE | B | 182 | −11.426 | −13.235 | −21.960 | 1.00 | 36.43 | 6 | C |
| ATOM | 3105 | O | PHE | B | 182 | −10.459 | −13.841 | −22.408 | 1.00 | 37.17 | 8 | O |
| ATOM | 3106 | N | GLN | B | 183 | −12.554 | −13.847 | −21.622 | 1.00 | 37.30 | 7 | N |
| ATOM | 3107 | CA | GLN | B | 183 | −12.657 | −15.315 | −21.637 | 1.00 | 37.89 | 6 | C |
| ATOM | 3108 | CB | GLN | B | 183 | −13.905 | −15.795 | −20.885 | 1.00 | 38.49 | 6 | C |
| ATOM | 3109 | CG | GLN | B | 183 | −13.600 | −16.691 | −19.676 | 1.00 | 42.03 | 6 | C |
| ATOM | 3110 | CD | GLN | B | 183 | −14.591 | −17.834 | −19.544 | 1.00 | 45.34 | 6 | C |
| ATOM | 3111 | OE1 | GLN | B | 183 | −15.503 | −17.964 | −20.365 | 1.00 | 47.51 | 8 | O |
| ATOM | 3112 | NE2 | GLN | B | 183 | −14.417 | −18.665 | −18.516 | 1.00 | 46.40 | 7 | N |
| ATOM | 3113 | C | GLN | B | 183 | −12.576 | −16.025 | −22.988 | 1.00 | 37.30 | 6 | C |
| ATOM | 3114 | O | GLN | B | 183 | −11.961 | −17.090 | −23.093 | 1.00 | 36.80 | 8 | O |
| ATOM | 3115 | N | ASP | B | 184 | −13.191 | −15.455 | −24.019 | 1.00 | 36.51 | 7 | N |
| ATOM | 3116 | CA | ASP | B | 184 | −13.233 | −16.129 | −25.314 | 1.00 | 35.92 | 6 | C |
| ATOM | 3117 | CB | ASP | B | 184 | −14.667 | −16.189 | −25.830 | 1.00 | 36.35 | 6 | C |
| ATOM | 3118 | CG | ASP | B | 184 | −15.262 | −14.814 | −26.017 | 1.00 | 36.30 | 6 | C |
| ATOM | 3119 | OD1 | ASP | B | 184 | −14.506 | −13.900 | −26.397 | 1.00 | 35.71 | 8 | O |
| ATOM | 3120 | OD2 | ASP | B | 184 | −16.456 | −14.545 | −25.775 | 1.00 | 39.16 | 8 | O |
| ATOM | 3121 | C | ASP | B | 184 | −12.333 | −15.493 | −26.357 | 1.00 | 35.04 | 6 | C |
| ATOM | 3122 | O | ASP | B | 184 | −12.241 | −15.984 | −27.475 | 1.00 | 35.28 | 8 | O |
| ATOM | 3123 | N | HIS | B | 185 | −11.689 | −14.388 | −26.001 | 1.00 | 34.06 | 7 | N |
| ATOM | 3124 | CA | HIS | B | 185 | −10.746 | −13.735 | −26.902 | 1.00 | 33.53 | 6 | C |
| ATOM | 3125 | CB | HIS | B | 185 | −9.706 | −14.749 | −27.362 | 1.00 | 34.21 | 6 | C |
| ATOM | 3126 | CG | HIS | B | 185 | −8.925 | −15.342 | −26.232 | 1.00 | 36.01 | 6 | C |
| ATOM | 3127 | ND1 | HIS | B | 185 | −8.240 | −14.567 | −25.321 | 1.00 | 37.51 | 7 | N |
| ATOM | 3128 | CE1 | HIS | B | 185 | −7.657 | −15.349 | −24.430 | 1.00 | 38.12 | 6 | C |
| ATOM | 3129 | NE2 | HIS | B | 185 | −7.946 | −16.603 | −24.726 | 1.00 | 38.61 | 7 | N |
| ATOM | 3130 | CD2 | HIS | B | 185 | −8.742 | −16.627 | −25.848 | 1.00 | 37.72 | 6 | C |
| ATOM | 3131 | C | HIS | B | 185 | −11.354 | −13.017 | −28.121 | 1.00 | 32.46 | 6 | C |
| ATOM | 3132 | O | HIS | B | 185 | −10.627 | −12.603 | −29.026 | 1.00 | 31.72 | 8 | O |
| ATOM | 3133 | N | THR | B | 186 | −12.674 | −12.876 | −28.149 | 1.00 | 31.83 | 7 | N |
| ATOM | 3134 | CA | THR | B | 186 | −13.302 | −12.146 | −29.250 | 1.00 | 31.41 | 6 | C |
| ATOM | 3135 | CB | THR | B | 186 | −14.777 | −12.539 | −29.430 | 1.00 | 31.18 | 6 | C |
| ATOM | 3136 | OG1 | THR | B | 186 | −15.477 | −12.376 | −28.196 | 1.00 | 30.58 | 8 | O |
| ATOM | 3137 | CG2 | THR | B | 186 | −14.921 | −14.034 | −29.737 | 1.00 | 31.70 | 6 | C |
| ATOM | 3138 | C | THR | B | 186 | −13.178 | −10.638 | −29.001 | 1.00 | 31.34 | 6 | C |
| ATOM | 3139 | O | THR | B | 186 | −13.050 | −10.200 | −27.855 | 1.00 | 31.21 | 8 | O |
| ATOM | 3140 | N | LYS | B | 187 | −13.212 | −9.852 | −30.075 | 1.00 | 30.85 | 7 | N |
| ATOM | 3141 | CA | LYS | B | 187 | −13.076 | −8.406 | −29.958 | 1.00 | 30.83 | 6 | C |
| ATOM | 3142 | CB | LYS | B | 187 | −11.626 | −7.977 | −30.210 | 1.00 | 31.19 | 6 | C |
| ATOM | 3143 | CG | LYS | B | 187 | −10.576 | −8.793 | −29.461 | 1.00 | 31.58 | 6 | C |
| ATOM | 3144 | CD | LYS | B | 187 | −9.185 | −8.345 | −29.866 | 1.00 | 34.32 | 6 | C |
| ATOM | 3145 | CE | LYS | B | 187 | −8.109 | −8.980 | −28.981 | 1.00 | 34.39 | 6 | C |
| ATOM | 3146 | NZ | LYS | B | 187 | −8.082 | −10.456 | −29.126 | 1.00 | 34.97 | 7 | N |
| ATOM | 3147 | C | LYS | B | 187 | −13.961 | −7.685 | −30.966 | 1.00 | 30.47 | 6 | C |
| ATOM | 3148 | O | LYS | B | 187 | −14.236 | −8.211 | −32.048 | 1.00 | 29.89 | 8 | O |
| ATOM | 3149 | N | LEU | B | 188 | −14.387 | −6.480 | −30.598 | 1.00 | 29.76 | 7 | N |
| ATOM | 3150 | CA | LEU | B | 188 | −15.140 | −5.618 | −31.493 | 1.00 | 30.16 | 6 | C |
| ATOM | 3151 | CB | LEU | B | 188 | −16.533 | −5.320 | −30.954 | 1.00 | 29.59 | 6 | C |
| ATOM | 3152 | CG | LEU | B | 188 | −17.545 | −6.424 | −30.679 | 1.00 | 30.75 | 6 | C |
| ATOM | 3153 | CD1 | LEU | B | 188 | −18.701 | −5.817 | −29.923 | 1.00 | 31.35 | 6 | C |
| ATOM | 3154 | CD2 | LEU | B | 188 | −18.039 | −7.056 | −31.959 | 1.00 | 32.85 | 6 | C |
| ATOM | 3155 | C | LEU | B | 188 | −14.406 | −4.292 | −31.639 | 1.00 | 30.15 | 6 | C |
| ATOM | 3156 | O | LEU | B | 188 | −13.838 | −3.775 | −30.678 | 1.00 | 30.39 | 8 | O |
| ATOM | 3157 | N | ILE | B | 189 | −14.404 | −3.753 | −32.845 | 1.00 | 30.49 | 7 | N |
| ATOM | 3158 | CA | ILE | B | 189 | −13.876 | −2.418 | −33.076 | 1.00 | 30.85 | 6 | C |
| ATOM | 3159 | CB | ILE | B | 189 | −12.663 | −2.446 | −34.002 | 1.00 | 30.93 | 6 | C |
| ATOM | 3160 | CG1 | ILE | B | 189 | −11.558 | −3.346 | −33.422 | 1.00 | 31.25 | 6 | C |
| ATOM | 3161 | CD1 | ILE | B | 189 | −10.629 | −3.901 | −34.473 | 1.00 | 33.02 | 6 | C |
| ATOM | 3162 | CG2 | ILE | B | 189 | −12.149 | −1.025 | −34.211 | 1.00 | 31.24 | 6 | C |
| ATOM | 3163 | C | ILE | B | 189 | −15.010 | −1.620 | −33.712 | 1.00 | 31.21 | 6 | C |
| ATOM | 3164 | O | ILE | B | 189 | −15.417 | −1.912 | −34.832 | 1.00 | 31.08 | 8 | O |
| ATOM | 3165 | N | LEU | B | 190 | −15.527 | −0.632 | −32.988 | 1.00 | 31.34 | 7 | N |
| ATOM | 3166 | CA | LEU | B | 190 | −16.656 | 0.159 | −33.478 | 1.00 | 31.95 | 6 | C |
| ATOM | 3167 | CB | LEU | B | 190 | −17.745 | 0.256 | −32.404 | 1.00 | 31.74 | 6 | C |
| ATOM | 3168 | CG | LEU | B | 190 | −18.423 | −1.062 | −31.992 | 1.00 | 31.40 | 6 | C |
| ATOM | 3169 | CD1 | LEU | B | 190 | −18.772 | −1.078 | −30.505 | 1.00 | 32.33 | 6 | C |
| ATOM | 3170 | CD2 | LEU | B | 190 | −19.668 | −1.321 | −32.823 | 1.00 | 30.49 | 6 | C |
| ATOM | 3171 | C | LEU | B | 190 | −16.216 | 1.553 | −33.908 | 1.00 | 32.26 | 6 | C |
| ATOM | 3172 | O | LEU | B | 190 | −15.495 | 2.233 | −33.187 | 1.00 | 32.36 | 8 | O |
| ATOM | 3173 | N | CYS | B | 191 | −16.648 | 1.970 | −35.093 | 1.00 | 32.91 | 7 | N |
| ATOM | 3174 | CA | CYS | B | 191 | −16.347 | 3.310 | −35.593 | 1.00 | 33.26 | 6 | C |
| ATOM | 3175 | CB | CYS | B | 191 | −15.517 | 3.243 | −36.873 | 1.00 | 33.24 | 6 | C |
| ATOM | 3176 | SG | CYS | B | 191 | −15.232 | 4.869 | −37.646 | 1.00 | 34.76 | 16 | S |
| ATOM | 3177 | C | CYS | B | 191 | −17.657 | 4.032 | −35.879 | 1.00 | 33.25 | 6 | C |
| ATOM | 3178 | O | CYS | B | 191 | −18.436 | 3.575 | −36.703 | 1.00 | 33.43 | 8 | O |
| ATOM | 3179 | N | PRO | B | 192 | −17.904 | 5.141 | −35.187 | 1.00 | 33.55 | 7 | N |
| ATOM | 3180 | CA | PRO | B | 192 | −19.135 | 5.919 | −35.361 | 1.00 | 34.06 | 6 | C |
| ATOM | 3181 | CB | PRO | B | 192 | −19.162 | 6.758 | −34.090 | 1.00 | 33.94 | 6 | C |
| ATOM | 3182 | CG | PRO | B | 192 | −17.714 | 7.062 | −33.888 | 1.00 | 33.71 | 6 | C |
| ATOM | 3183 | CD | PRO | B | 192 | −17.042 | 5.729 | −34.146 | 1.00 | 33.40 | 6 | C |
| ATOM | 3184 | C | PRO | B | 192 | −19.128 | 6.817 | −36.600 | 1.00 | 34.90 | 6 | C |
| ATOM | 3185 | O | PRO | B | 192 | −20.185 | 7.339 | −36.978 | 1.00 | 34.87 | 8 | O |
| ATOM | 3186 | N | LEU | B | 193 | −17.959 | 7.003 | −37.212 | 1.00 | 35.55 | 7 | N |
| ATOM | 3187 | CA | LEU | B | 193 | −17.849 | 7.806 | −38.429 | 1.00 | 36.44 | 6 | C |
| ATOM | 3188 | CB | LEU | B | 193 | −16.401 | 8.228 | −38.670 | 1.00 | 36.83 | 6 | C |
| ATOM | 3189 | CG | LEU | B | 193 | −15.830 | 9.480 | −38.002 | 1.00 | 38.55 | 6 | C |
| ATOM | 3190 | CD1 | LEU | B | 193 | −16.082 | 9.509 | −36.509 | 1.00 | 38.97 | 6 | C |
| ATOM | 3191 | CD2 | LEU | B | 193 | −14.338 | 9.579 | −38.290 | 1.00 | 39.57 | 6 | C |
| ATOM | 3192 | C | LEU | B | 193 | −18.339 | 6.986 | −39.609 | 1.00 | 36.47 | 6 | C |
| ATOM | 3193 | O | LEU | B | 193 | −19.032 | 7.490 | −40.488 | 1.00 | 37.34 | 8 | O |
| ATOM | 3194 | N | MET | B | 194 | −17.987 | 5.706 | −39.617 | 1.00 | 36.22 | 7 | N |
| ATOM | 3195 | CA | MET | B | 194 | −18.396 | 4.804 | −40.684 | 1.00 | 35.99 | 6 | C |
| ATOM | 3196 | CB | MET | B | 194 | −17.258 | 3.833 | −41.006 | 1.00 | 36.75 | 6 | C |
| ATOM | 3197 | CG | MET | B | 194 | −15.974 | 4.507 | −41.482 | 1.00 | 40.37 | 6 | C |
| ATOM | 3198 | SD | MET | B | 194 | −16.263 | 5.440 | −42.975 | 1.00 | 48.21 | 16 | S |
| ATOM | 3199 | CE | MET | B | 194 | −16.542 | 4.127 | −44.115 | 1.00 | 45.23 | 6 | C |
| ATOM | 3200 | C | MET | B | 194 | −19.637 | 4.001 | −40.297 | 1.00 | 34.72 | 6 | C |
| ATOM | 3201 | O | MET | B | 194 | −20.161 | 3.232 | −41.110 | 1.00 | 34.69 | 8 | O |
| ATOM | 3202 | N | ALA | B | 195 | −20.102 | 4.187 | −39.061 | 1.00 | 32.98 | 7 | N |
| ATOM | 3203 | CA | ALA | B | 195 | −21.232 | 3.421 | −38.532 | 1.00 | 31.57 | 6 | C |
| ATOM | 3204 | CB | ALA | B | 195 | −22.562 | 3.901 | −39.143 | 1.00 | 31.57 | 6 | C |
| ATOM | 3205 | C | ALA | B | 195 | −21.001 | 1.938 | −38.810 | 1.00 | 30.28 | 6 | C |
| ATOM | 3206 | O | ALA | B | 195 | −21.856 | 1.254 | −39.373 | 1.00 | 29.74 | 8 | O |
| ATOM | 3207 | N | ALA | B | 196 | −19.836 | 1.447 | −38.394 | 1.00 | 29.57 | 7 | N |
| ATOM | 3208 | CA | ALA | B | 196 | −19.434 | 0.076 | −38.667 | 1.00 | 29.18 | 6 | C |
| ATOM | 3209 | CB | ALA | B | 196 | −18.423 | 0.049 | −39.814 | 1.00 | 29.60 | 6 | C |
| ATOM | 3210 | C | ALA | B | 196 | −18.852 | −0.646 | −37.458 | 1.00 | 29.28 | 6 | C |
| ATOM | 3211 | O | ALA | B | 196 | −18.476 | −0.023 | −36.457 | 1.00 | 29.26 | 8 | O |
| ATOM | 3212 | N | VAL | B | 197 | −18.774 | −1.965 | −37.574 | 1.00 | 28.98 | 7 | N |
| ATOM | 3213 | CA | VAL | B | 197 | −18.202 | −2.794 | −36.526 | 1.00 | 28.77 | 6 | C |
| ATOM | 3214 | CB | VAL | B | 197 | −19.281 | −3.482 | −35.649 | 1.00 | 28.71 | 6 | C |
| ATOM | 3215 | CG1 | VAL | B | 197 | −20.183 | −4.404 | −36.484 | 1.00 | 28.89 | 6 | C |
| ATOM | 3216 | CG2 | VAL | B | 197 | −18.624 | −4.270 | −34.509 | 1.00 | 29.06 | 6 | C |
| ATOM | 3217 | C | VAL | B | 197 | −17.329 | −3.851 | −37.170 | 1.00 | 29.08 | 6 | C |
| ATOM | 3218 | O | VAL | B | 197 | −17.703 | −4.458 | −38.172 | 1.00 | 28.66 | 8 | O |
| ATOM | 3219 | N | THR | B | 198 | −16.147 | −4.045 | −36.599 | 1.00 | 29.05 | 7 | N |
| ATOM | 3220 | CA | THR | B | 198 | −15.260 | −5.115 | −37.017 | 1.00 | 29.47 | 6 | C |
| ATOM | 3221 | CB | THR | B | 198 | −13.830 | −4.589 | −37.150 | 1.00 | 29.50 | 6 | C |
| ATOM | 3222 | OG1 | THR | B | 198 | −13.763 | −3.713 | −38.279 | 1.00 | 30.50 | 8 | O |
| ATOM | 3223 | CG2 | THR | B | 198 | −12.858 | −5.715 | −37.527 | 1.00 | 29.77 | 6 | C |
| ATOM | 3224 | C | THR | B | 198 | −15.334 | −6.159 | −35.923 | 1.00 | 29.99 | 6 | C |
| ATOM | 3225 | O | THR | B | 198 | −15.176 | −5.832 | −34.745 | 1.00 | 29.34 | 8 | O |
| ATOM | 3226 | N | TYR | B | 199 | −15.615 | −7.400 | −36.308 | 1.00 | 29.93 | 7 | N |
| ATOM | 3227 | CA | TYR | B | 199 | −15.720 | −8.472 | −35.341 | 1.00 | 30.91 | 6 | C |
| ATOM | 3228 | CB | TYR | B | 199 | −17.046 | −9.224 | −35.521 | 1.00 | 30.44 | 6 | C |
| ATOM | 3229 | CG | TYR | B | 199 | −17.254 | −10.364 | −34.558 | 1.00 | 30.96 | 6 | C |
| ATOM | 3230 | CD1 | TYR | B | 199 | −16.804 | −10.285 | −33.247 | 1.00 | 31.88 | 6 | C |
| ATOM | 3231 | CE1 | TYR | B | 199 | −16.991 | −11.325 | −32.365 | 1.00 | 32.55 | 6 | C |
| ATOM | 3232 | CZ | TYR | B | 199 | −17.647 | −12.459 | −32.774 | 1.00 | 33.17 | 6 | C |
| ATOM | 3233 | OH | TYR | B | 199 | −17.840 | −13.489 | −31.878 | 1.00 | 36.31 | 8 | O |
| ATOM | 3234 | CE2 | TYR | B | 199 | −18.110 | −12.564 | −34.067 | 1.00 | 33.56 | 6 | C |
| ATOM | 3235 | CD2 | TYR | B | 199 | −17.915 | −11.519 | −34.952 | 1.00 | 32.17 | 6 | C |
| ATOM | 3236 | C | TYR | B | 199 | −14.554 | −9.419 | −35.560 | 1.00 | 31.41 | 6 | C |
| ATOM | 3237 | O | TYR | B | 199 | −14.332 | −9.887 | −36.673 | 1.00 | 31.16 | 8 | O |
| ATOM | 3238 | N | ILE | B | 200 | −13.794 | −9.662 | −34.502 | 1.00 | 32.55 | 7 | N |
| ATOM | 3239 | CA | ILE | B | 200 | −12.714 | −10.635 | −34.534 | 1.00 | 33.59 | 6 | C |
| ATOM | 3240 | CB | ILE | B | 200 | −11.414 | −10.031 | −33.962 | 1.00 | 33.63 | 6 | C |
| ATOM | 3241 | CG1 | ILE | B | 200 | −10.948 | −8.870 | −34.843 | 1.00 | 33.74 | 6 | C |
| ATOM | 3242 | CD1 | ILE | B | 200 | −9.712 | −8.133 | −34.330 | 1.00 | 35.27 | 6 | C |
| ATOM | 3243 | CG2 | ILE | B | 200 | −10.325 | −11.108 | −33.866 | 1.00 | 33.81 | 6 | C |
| ATOM | 3244 | C | ILE | B | 200 | −13.198 | −11.801 | −33.693 | 1.00 | 34.45 | 6 | C |
| ATOM | 3245 | O | ILE | B | 200 | −13.412 | −11.656 | −32.496 | 1.00 | 34.78 | 8 | O |
| ATOM | 3246 | N | ASP | B | 201 | −13.410 | −12.953 | −34.318 | 1.00 | 35.60 | 7 | N |
| ATOM | 3247 | CA | ASP | B | 201 | −13.973 | −14.086 | −33.597 | 1.00 | 36.61 | 6 | C |
| ATOM | 3248 | CB | ASP | B | 201 | −14.868 | −14.925 | −34.508 | 1.00 | 36.72 | 6 | C |
| ATOM | 3249 | CG | ASP | B | 201 | −14.106 | −15.575 | −35.654 | 1.00 | 37.71 | 6 | C |
| ATOM | 3250 | OD1 | ASP | B | 201 | −12.851 | −15.613 | −35.626 | 1.00 | 37.60 | 8 | O |
| ATOM | 3251 | OD2 | ASP | B | 201 | −14.696 | −16.088 | −36.627 | 1.00 | 38.71 | 8 | O |
| ATOM | 3252 | C | ASP | B | 201 | −12.908 | −14.948 | −32.924 | 1.00 | 37.15 | 6 | C |
| ATOM | 3253 | O | ASP | B | 201 | −11.729 | −14.620 | −32.962 | 1.00 | 37.08 | 8 | O |
| ATOM | 3254 | N | GLU | B | 202 | −13.335 | −16.045 | −32.311 | 1.00 | 38.32 | 7 | N |
| ATOM | 3255 | CA | GLU | B | 202 | −12.408 | −16.924 | −31.599 | 1.00 | 39.84 | 6 | C |
| ATOM | 3256 | CB | GLU | B | 202 | −13.148 | −17.883 | −30.658 | 1.00 | 40.25 | 6 | C |
| ATOM | 3257 | CG | GLU | B | 202 | −14.367 | −18.567 | −31.256 | 1.00 | 42.60 | 6 | C |
| ATOM | 3258 | CD | GLU | B | 202 | −15.614 | −17.703 | −31.162 | 1.00 | 45.06 | 6 | C |
| ATOM | 3259 | OE1 | GLU | B | 202 | −16.208 | −17.626 | −30.059 | 1.00 | 46.48 | 8 | O |
| ATOM | 3260 | OE2 | GLU | B | 202 | −15.989 | −17.097 | −32.186 | 1.00 | 44.47 | 8 | O |
| ATOM | 3261 | C | GLU | B | 202 | −11.434 | −17.690 | −32.503 | 1.00 | 40.07 | 6 | C |
| ATOM | 3262 | O | GLU | B | 202 | −10.446 | −18.235 | −32.013 | 1.00 | 40.52 | 8 | O |
| ATOM | 3263 | N | LYS | B | 203 | −11.700 | −17.730 | −33.808 | 1.00 | 40.52 | 7 | N |
| ATOM | 3264 | CA | LYS | B | 203 | −10.784 | −18.372 | −34.754 | 1.00 | 41.15 | 6 | C |
| ATOM | 3265 | CB | LYS | B | 203 | −11.518 | −18.902 | −35.986 | 1.00 | 41.26 | 6 | C |
| ATOM | 3266 | CG | LYS | B | 203 | −12.568 | −19.953 | −35.768 | 1.00 | 42.20 | 6 | C |
| ATOM | 3267 | CD | LYS | B | 203 | −13.049 | −20.405 | −37.141 | 1.00 | 43.91 | 6 | C |
| ATOM | 3268 | CE | LYS | B | 203 | −14.421 | −21.032 | −37.111 | 1.00 | 45.01 | 6 | C |
| ATOM | 3269 | NZ | LYS | B | 203 | −14.983 | −21.106 | −38.487 | 1.00 | 45.80 | 7 | N |
| ATOM | 3270 | C | LYS | B | 203 | −9.806 | −17.329 | −35.254 | 1.00 | 41.28 | 6 | C |
| ATOM | 3271 | O | LYS | B | 203 | −8.943 | −17.619 | −36.080 | 1.00 | 41.11 | 8 | O |
| ATOM | 3272 | N | ARG | B | 204 | −9.971 | −16.106 | −34.761 | 1.00 | 41.53 | 7 | N |
| ATOM | 3273 | CA | ARG | B | 204 | −9.168 | −14.958 | −35.179 | 1.00 | 42.04 | 6 | C |
| ATOM | 3274 | CB | ARG | B | 204 | −7.666 | −15.235 | −35.094 | 1.00 | 42.26 | 6 | C |
| ATOM | 3275 | CG | ARG | B | 204 | −7.154 | −15.387 | −33.682 | 1.00 | 44.06 | 6 | C |
| ATOM | 3276 | CD | ARG | B | 204 | −5.672 | −15.068 | −33.530 | 1.00 | 47.27 | 6 | C |
| ATOM | 3277 | NE | ARG | B | 204 | −5.037 | −15.848 | −32.470 | 1.00 | 49.18 | 7 | N |
| ATOM | 3278 | CZ | ARG | B | 204 | −5.371 | −15.794 | −31.189 | 1.00 | 50.09 | 6 | C |
| ATOM | 3279 | NH1 | ARG | B | 204 | −6.347 | −14.989 | −30.787 | 1.00 | 51.10 | 7 | N |
| ATOM | 3280 | NH2 | ARG | B | 204 | −4.729 | −16.549 | −30.302 | 1.00 | 50.21 | 7 | N |
| ATOM | 3281 | C | ARG | B | 204 | −9.552 | −14.443 | −36.564 | 1.00 | 41.94 | 6 | C |
| ATOM | 3282 | O | ARG | B | 204 | −8.838 | −13.641 | −37.163 | 1.00 | 41.39 | 8 | O |
| ATOM | 3283 | N | ASP | B | 205 | −10.680 | −14.912 | −37.082 | 1.00 | 42.30 | 7 | N |
| ATOM | 3284 | CA | ASP | B | 205 | −11.159 | −14.383 | −38.343 | 1.00 | 42.63 | 6 | C |
| ATOM | 3285 | CB | ASP | B | 205 | −12.084 | −15.355 | −39.059 | 1.00 | 43.15 | 6 | C |
| ATOM | 3286 | CG | ASP | B | 205 | −11.363 | −16.147 | −40.116 | 1.00 | 45.20 | 6 | C |
| ATOM | 3287 | OD1 | ASP | B | 205 | −11.487 | −15.791 | −41.308 | 1.00 | 48.54 | 8 | O |
| ATOM | 3288 | OD2 | ASP | B | 205 | −10.636 | −17.126 | −39.847 | 1.00 | 46.34 | 8 | O |
| ATOM | 3289 | C | ASP | B | 205 | −11.847 | −13.064 | −38.078 | 1.00 | 42.42 | 6 | C |
| ATOM | 3290 | O | ASP | B | 205 | −12.376 | −12.828 | −36.988 | 1.00 | 42.54 | 8 | O |
| ATOM | 3291 | N | PHE | B | 206 | −11.827 | −12.202 | −39.080 | 1.00 | 41.94 | 7 | N |
| ATOM | 3292 | CA | PHE | B | 206 | −12.350 | −10.863 | −38.928 | 1.00 | 41.63 | 6 | C |
| ATOM | 3293 | CB | PHE | B | 206 | −11.191 | −9.880 | −38.826 | 1.00 | 41.67 | 6 | C |
| ATOM | 3294 | CG | PHE | B | 206 | −10.306 | −9.887 | −40.032 | 1.00 | 43.86 | 6 | C |
| ATOM | 3295 | CD1 | PHE | B | 206 | −10.387 | −8.874 | −40.974 | 1.00 | 45.15 | 6 | C |
| ATOM | 3296 | CE1 | PHE | B | 206 | −9.581 | −8.890 | −42.094 | 1.00 | 45.57 | 6 | C |
| ATOM | 3297 | CZ | PHE | B | 206 | −8.691 | −9.931 | −42.294 | 1.00 | 46.16 | 6 | C |
| ATOM | 3298 | CE2 | PHE | B | 206 | −8.608 | −10.952 | −41.367 | 1.00 | 46.38 | 6 | C |
| ATOM | 3299 | CD2 | PHE | B | 206 | −9.416 | −10.929 | −40.245 | 1.00 | 45.29 | 6 | C |
| ATOM | 3300 | C | PHE | B | 206 | −13.214 | −10.475 | −40.111 | 1.00 | 40.74 | 6 | C |
| ATOM | 3301 | O | PHE | B | 206 | −12.981 | −10.896 | −41.253 | 1.00 | 40.86 | 8 | O |
| ATOM | 3302 | N | ARG | B | 207 | −14.203 | −9.644 | −39.828 | 1.00 | 39.32 | 7 | N |
| ATOM | 3303 | CA | ARG | B | 207 | −15.099 | −9.141 | −40.843 | 1.00 | 38.06 | 6 | C |
| ATOM | 3304 | CB | ARG | B | 207 | −16.283 | −10.086 | −41.014 | 1.00 | 38.93 | 6 | C |
| ATOM | 3305 | CG | ARG | B | 207 | −15.921 | −11.425 | −41.649 | 1.00 | 41.23 | 6 | C |
| ATOM | 3306 | CD | ARG | B | 207 | −15.806 | −11.365 | −43.161 | 1.00 | 45.10 | 6 | C |
| ATOM | 3307 | NE | ARG | B | 207 | −15.000 | −12.445 | −43.720 | 1.00 | 47.95 | 7 | N |
| ATOM | 3308 | CZ | ARG | B | 207 | −15.354 | −13.725 | −43.735 | 1.00 | 49.42 | 6 | C |
| ATOM | 3309 | NH1 | ARG | B | 207 | −16.504 | −14.113 | −43.204 | 1.00 | 50.39 | 7 | N |
| ATOM | 3310 | NH2 | ARG | B | 207 | −14.548 | −14.624 | −44.281 | 1.00 | 49.46 | 7 | N |
| ATOM | 3311 | C | ARG | B | 207 | −15.575 | −7.786 | −40.365 | 1.00 | 36.54 | 6 | C |
| ATOM | 3312 | O | ARG | B | 207 | −15.753 | −7.576 | −39.167 | 1.00 | 35.56 | 8 | O |
| ATOM | 3313 | N | THR | B | 208 | −15.750 | −6.864 | −41.299 | 1.00 | 34.48 | 7 | N |
| ATOM | 3314 | CA | THR | B | 208 | −16.203 | −5.526 | −40.975 | 1.00 | 33.17 | 6 | C |
| ATOM | 3315 | CB | THR | B | 208 | −15.233 | −4.497 | −41.561 | 1.00 | 33.38 | 6 | C |
| ATOM | 3316 | OG1 | THR | B | 208 | −13.963 | −4.594 | −40.887 | 1.00 | 32.93 | 8 | O |
| ATOM | 3317 | CG2 | THR | B | 208 | −15.715 | −3.091 | −41.253 | 1.00 | 33.01 | 6 | C |
| ATOM | 3318 | C | THR | B | 208 | −17.590 | −5.349 | −41.577 | 1.00 | 32.35 | 6 | C |
| ATOM | 3319 | O | THR | B | 208 | −17.776 | −5.583 | −42.777 | 1.00 | 32.13 | 8 | O |
| ATOM | 3320 | N | TYR | B | 209 | −18.547 | −4.929 | −40.751 | 1.00 | 30.97 | 7 | N |
| ATOM | 3321 | CA | TYR | B | 209 | −19.938 | −4.776 | −41.175 | 1.00 | 30.20 | 6 | C |
| ATOM | 3322 | CB | TYR | B | 209 | −20.846 | −5.716 | −40.362 | 1.00 | 29.85 | 6 | C |
| ATOM | 3323 | CG | TYR | B | 209 | −20.446 | −7.168 | −40.380 | 1.00 | 30.61 | 6 | C |
| ATOM | 3324 | CD1 | TYR | B | 209 | −19.672 | −7.719 | −39.351 | 1.00 | 30.41 | 6 | C |
| ATOM | 3325 | CE1 | TYR | B | 209 | −19.302 | −9.053 | −39.377 | 1.00 | 31.94 | 6 | C |
| ATOM | 3326 | CZ | TYR | B | 209 | −19.718 | −9.844 | −40.433 | 1.00 | 31.53 | 6 | C |
| ATOM | 3327 | OH | TYR | B | 209 | −19.368 | −11.171 | −40.498 | 1.00 | 30.94 | 8 | O |
| ATOM | 3328 | CE2 | TYR | B | 209 | −20.476 | −9.310 | −41.455 | 1.00 | 31.95 | 6 | C |
| ATOM | 3329 | CD2 | TYR | B | 209 | −20.830 | −7.989 | −41.424 | 1.00 | 31.27 | 6 | C |
| ATOM | 3330 | C | TYR | B | 209 | −20.480 | −3.377 | −40.969 | 1.00 | 29.27 | 6 | C |
| ATOM | 3331 | O | TYR | B | 209 | −20.145 | −2.726 | −39.989 | 1.00 | 29.32 | 8 | O |
| ATOM | 3332 | N | ARG | B | 210 | −21.345 | −2.925 | −41.878 | 1.00 | 28.16 | 7 | N |
| ATOM | 3333 | CA | ARG | B | 210 | −22.070 | −1.676 | −41.658 | 1.00 | 27.88 | 6 | C |
| ATOM | 3334 | CB | ARG | B | 210 | −22.643 | −1.138 | −42.969 | 1.00 | 27.74 | 6 | C |
| ATOM | 3335 | CG | ARG | B | 210 | −21.594 | −0.722 | −43.996 | 1.00 | 29.12 | 6 | C |
| ATOM | 3336 | CD | ARG | B | 210 | −20.835 | 0.545 | −43.644 | 1.00 | 30.87 | 6 | C |
| ATOM | 3337 | NE | ARG | B | 210 | −20.084 | 1.055 | −44.796 | 1.00 | 30.84 | 7 | N |
| ATOM | 3338 | CZ | ARG | B | 210 | −19.609 | 2.288 | −44.889 | 1.00 | 31.64 | 6 | C |
| ATOM | 3339 | NH1 | ARG | B | 210 | −19.792 | 3.152 | −43.899 | 1.00 | 31.10 | 7 | N |
| ATOM | 3340 | NH2 | ARG | B | 210 | −18.953 | 2.671 | −45.983 | 1.00 | 31.71 | 7 | N |
| ATOM | 3341 | C | ARG | B | 210 | −23.216 | −1.973 | −40.701 | 1.00 | 27.62 | 6 | C |
| ATOM | 3342 | O | ARG | B | 210 | −24.034 | −2.859 | −40.967 | 1.00 | 27.36 | 8 | O |
| ATOM | 3343 | N | LEU | B | 211 | −23.311 | −1.219 | −39.609 | 1.00 | 27.26 | 7 | N |
| ATOM | 3344 | CA | LEU | B | 211 | −24.358 | −1.463 | −38.613 | 1.00 | 27.06 | 6 | C |
| ATOM | 3345 | CB | LEU | B | 211 | −24.214 | −0.482 | −37.445 | 1.00 | 26.85 | 6 | C |
| ATOM | 3346 | CG | LEU | B | 211 | −22.968 | −0.712 | −36.575 | 1.00 | 26.35 | 6 | C |
| ATOM | 3347 | CD1 | LEU | B | 211 | −22.809 | 0.428 | −35.570 | 1.00 | 27.27 | 6 | C |
| ATOM | 3348 | CD2 | LEU | B | 211 | −23.061 | −2.065 | −35.860 | 1.00 | 27.54 | 6 | C |
| ATOM | 3349 | C | LEU | B | 211 | −25.774 | −1.379 | −39.201 | 1.00 | 27.36 | 6 | C |
| ATOM | 3350 | O | LEU | B | 211 | −26.637 | −2.200 | −38.891 | 1.00 | 26.70 | 8 | O |
| ATOM | 3351 | N | SER | B | 212 | −26.013 | −0.377 | −40.039 | 1.00 | 27.73 | 7 | N |
| ATOM | 3352 | CA | SER | B | 212 | −27.332 | −0.234 | −40.668 | 1.00 | 28.63 | 6 | C |
| ATOM | 3353 | CB | SER | B | 212 | −27.441 | 1.079 | −41.449 | 1.00 | 28.91 | 6 | C |
| ATOM | 3354 | OG | SER | B | 212 | −27.316 | 2.220 | −40.608 | 1.00 | 32.24 | 8 | O |
| ATOM | 3355 | C | SER | B | 212 | −27.653 | −1.420 | −41.582 | 1.00 | 28.29 | 6 | C |
| ATOM | 3356 | O | SER | B | 212 | −28.819 | −1.789 | −41.763 | 1.00 | 28.79 | 8 | O |
| ATOM | 3357 | N | LEU | B | 213 | −26.624 | −2.010 | −42.183 | 1.00 | 28.05 | 7 | N |
| ATOM | 3358 | CA | LEU | B | 213 | −26.835 | −3.180 | −43.029 | 1.00 | 27.68 | 6 | C |
| ATOM | 3359 | CB | LEU | B | 213 | −25.668 | −3.372 | −43.994 | 1.00 | 27.38 | 6 | C |
| ATOM | 3360 | CG | LEU | B | 213 | −25.551 | −2.331 | −45.113 | 1.00 | 26.68 | 6 | C |
| ATOM | 3361 | CD1 | LEU | B | 213 | −24.458 | −2.754 | −46.068 | 1.00 | 26.26 | 6 | C |
| ATOM | 3362 | CD2 | LEU | B | 213 | −26.893 | −2.195 | −45.857 | 1.00 | 26.57 | 6 | C |
| ATOM | 3363 | C | LEU | B | 213 | −27.095 | −4.462 | −42.216 | 1.00 | 28.03 | 6 | C |
| ATOM | 3364 | O | LEU | B | 213 | −27.747 | −5.397 | −42.695 | 1.00 | 27.81 | 8 | O |
| ATOM | 3365 | N | LEU | B | 214 | −26.560 | −4.532 | −41.001 | 1.00 | 27.99 | 7 | N |
| ATOM | 3366 | CA | LEU | B | 214 | −26.835 | −5.696 | −40.163 | 1.00 | 28.19 | 6 | C |
| ATOM | 3367 | CB | LEU | B | 214 | −25.940 | −5.723 | −38.914 | 1.00 | 27.94 | 6 | C |
| ATOM | 3368 | CG | LEU | B | 214 | −24.447 | −6.003 | −39.141 | 1.00 | 28.61 | 6 | C |
| ATOM | 3369 | CD1 | LEU | B | 214 | −23.665 | −5.834 | −37.824 | 1.00 | 29.52 | 6 | C |
| ATOM | 3370 | CD2 | LEU | B | 214 | −24.214 | −7.392 | −39.722 | 1.00 | 28.82 | 6 | C |
| ATOM | 3371 | C | LEU | B | 214 | −28.312 | −5.679 | −39.788 | 1.00 | 28.08 | 6 | C |
| ATOM | 3372 | O | LEU | B | 214 | −28.947 | −6.720 | −39.636 | 1.00 | 28.21 | 8 | O |
| ATOM | 3373 | N | GLU | B | 215 | −28.858 | −4.476 | −39.673 | 1.00 | 29.11 | 7 | N |
| ATOM | 3374 | CA | GLU | B | 215 | −30.260 | −4.263 | −39.352 | 1.00 | 30.18 | 6 | C |
| ATOM | 3375 | CB | GLU | B | 215 | −30.453 | −2.758 | −39.187 | 1.00 | 30.65 | 6 | C |
| ATOM | 3376 | CG | GLU | B | 215 | −31.844 | −2.267 | −38.869 | 1.00 | 33.74 | 6 | C |
| ATOM | 3377 | CD | GLU | B | 215 | −31.793 | −0.878 | −38.253 | 1.00 | 37.26 | 6 | C |
| ATOM | 3378 | OE1 | GLU | B | 215 | −30.680 | −0.444 | −37.846 | 1.00 | 38.83 | 8 | O |
| ATOM | 3379 | OE2 | GLU | B | 215 | −32.852 | −0.223 | −38.186 | 1.00 | 39.07 | 8 | O |
| ATOM | 3380 | C | GLU | B | 215 | −31.190 | −4.810 | −40.442 | 1.00 | 30.23 | 6 | C |
| ATOM | 3381 | O | GLU | B | 215 | −32.238 | −5.425 | −40.171 | 1.00 | 30.85 | 8 | O |
| ATOM | 3382 | N | GLU | B | 216 | −30.792 | −4.601 | −41.685 | 1.00 | 29.38 | 7 | N |
| ATOM | 3383 | CA | GLU | B | 216 | −31.588 | −5.031 | −42.815 | 1.00 | 29.69 | 6 | C |
| ATOM | 3384 | CB | GLU | B | 216 | −31.238 | −4.165 | −44.036 | 1.00 | 29.56 | 6 | C |
| ATOM | 3385 | CG | GLU | B | 216 | −31.732 | −2.726 | −43.962 | 1.00 | 31.01 | 6 | C |
| ATOM | 3386 | CD | GLU | B | 216 | −33.244 | −2.621 | −43.913 | 1.00 | 32.22 | 6 | C |
| ATOM | 3387 | OE1 | GLU | B | 216 | −33.815 | −2.644 | −42.796 | 1.00 | 35.17 | 8 | O |
| ATOM | 3388 | OE2 | GLU | B | 216 | −33.867 | −2.525 | −44.989 | 1.00 | 32.31 | 8 | O |
| ATOM | 3389 | C | GLU | B | 216 | −31.405 | −6.514 | −43.155 | 1.00 | 29.31 | 6 | C |
| ATOM | 3390 | O | GLU | B | 216 | −32.372 | −7.203 | −43.479 | 1.00 | 30.41 | 8 | O |
| ATOM | 3391 | N | TYR | B | 217 | −30.180 | −7.013 | −43.056 | 1.00 | 29.30 | 7 | N |
| ATOM | 3392 | CA | TYR | B | 217 | −29.882 | −8.370 | −43.499 | 1.00 | 29.29 | 6 | C |
| ATOM | 3393 | CB | TYR | B | 217 | −28.592 | −8.380 | −44.321 | 1.00 | 29.39 | 6 | C |
| ATOM | 3394 | CG | TYR | B | 217 | −28.745 | −7.699 | −45.664 | 1.00 | 29.10 | 6 | C |
| ATOM | 3395 | CD1 | TYR | B | 217 | −28.194 | −6.449 | −45.891 | 1.00 | 29.63 | 6 | C |
| ATOM | 3396 | CE1 | TYR | B | 217 | −28.339 | −5.814 | −47.126 | 1.00 | 29.43 | 6 | C |
| ATOM | 3397 | CZ | TYR | B | 217 | −29.042 | −6.431 | −48.146 | 1.00 | 31.15 | 6 | C |
| ATOM | 3398 | OH | TYR | B | 217 | −29.169 | −5.788 | −49.369 | 1.00 | 31.03 | 8 | O |
| ATOM | 3399 | CE2 | TYR | B | 217 | −29.604 | −7.672 | −47.947 | 1.00 | 31.19 | 6 | C |
| ATOM | 3400 | CD2 | TYR | B | 217 | −29.461 | −8.301 | −46.699 | 1.00 | 30.94 | 6 | C |
| ATOM | 3401 | C | TYR | B | 217 | −29.807 | −9.420 | −42.399 | 1.00 | 29.70 | 6 | C |
| ATOM | 3402 | O | TYR | B | 217 | −29.875 | −10.616 | −42.678 | 1.00 | 29.44 | 8 | O |
| ATOM | 3403 | N | GLY | B | 218 | −29.648 | −8.961 | −41.163 | 1.00 | 30.30 | 7 | N |
| ATOM | 3404 | CA | GLY | B | 218 | −29.556 | −9.833 | −40.008 | 1.00 | 30.67 | 6 | C |
| ATOM | 3405 | C | GLY | B | 218 | −28.126 | −10.233 | −39.703 | 1.00 | 31.35 | 6 | C |
| ATOM | 3406 | O | GLY | B | 218 | −27.209 | −9.917 | −40.466 | 1.00 | 30.66 | 8 | O |
| ATOM | 3407 | N | CYS | B | 219 | −27.930 | −10.913 | −38.572 | 1.00 | 31.71 | 7 | N |
| ATOM | 3408 | CA | CYS | B | 219 | −26.612 | −11.439 | −38.224 | 1.00 | 32.39 | 6 | C |
| ATOM | 3409 | CB | CYS | B | 219 | −25.646 | −10.339 | −37.797 | 1.00 | 32.73 | 6 | C |
| ATOM | 3410 | SG | CYS | B | 219 | −25.858 | −9.654 | −36.140 | 1.00 | 34.72 | 16 | S |
| ATOM | 3411 | C | CYS | B | 219 | −26.697 | −12.566 | −37.193 | 1.00 | 32.51 | 6 | C |
| ATOM | 3412 | O | CYS | B | 219 | −27.754 | −12.803 | −36.628 | 1.00 | 32.57 | 8 | O |
| ATOM | 3413 | N | CYS | B | 220 | −25.587 | −13.262 | −36.970 | 1.00 | 32.50 | 7 | N |
| ATOM | 3414 | CA | CYS | B | 220 | −25.554 | −14.380 | −36.027 | 1.00 | 32.97 | 6 | C |
| ATOM | 3415 | CB | CYS | B | 220 | −24.231 | −15.146 | −36.150 | 1.00 | 33.12 | 6 | C |
| ATOM | 3416 | SG | CYS | B | 220 | −22.768 | −14.149 | −35.739 | 1.00 | 37.64 | 16 | S |
| ATOM | 3417 | C | CYS | B | 220 | −25.690 | −13.925 | −34.584 | 1.00 | 32.49 | 6 | C |
| ATOM | 3418 | O | CYS | B | 220 | −25.447 | −12.759 | −34.262 | 1.00 | 31.34 | 8 | O |
| ATOM | 3419 | N | LYS | B | 221 | −26.046 | −14.871 | −33.717 | 1.00 | 32.02 | 7 | N |
| ATOM | 3420 | CA | LYS | B | 221 | −26.174 | −14.607 | −32.286 | 1.00 | 32.77 | 6 | C |
| ATOM | 3421 | CB | LYS | B | 221 | −26.625 | −15.876 | −31.552 | 1.00 | 32.93 | 6 | C |
| ATOM | 3422 | CG | LYS | B | 221 | −28.120 | −16.043 | −31.463 | 1.00 | 36.27 | 6 | C |
| ATOM | 3423 | CD | LYS | B | 221 | −28.473 | −17.215 | −30.528 | 1.00 | 39.66 | 6 | C |
| ATOM | 3424 | CE | LYS | B | 221 | −29.913 | −17.111 | −30.032 | 1.00 | 41.43 | 6 | C |
| ATOM | 3425 | NZ | LYS | B | 221 | −30.246 | −18.141 | −29.000 | 1.00 | 43.73 | 7 | N |
| ATOM | 3426 | C | LYS | B | 221 | −24.868 | −14.109 | −31.674 | 1.00 | 32.01 | 6 | C |
| ATOM | 3427 | O | LYS | B | 221 | −24.887 | −13.267 | −30.777 | 1.00 | 32.02 | 8 | O |
| ATOM | 3428 | N | GLU | B | 222 | −23.749 | −14.666 | −32.136 | 1.00 | 31.92 | 7 | N |
| ATOM | 3429 | CA | GLU | B | 222 | −22.404 | −14.273 | −31.700 | 1.00 | 31.80 | 6 | C |
| ATOM | 3430 | CB | GLU | B | 222 | −21.364 | −14.845 | −32.672 | 1.00 | 32.80 | 6 | C |
| ATOM | 3431 | CG | GLU | B | 222 | −20.442 | −15.939 | −32.158 | 1.00 | 37.22 | 6 | C |
| ATOM | 3432 | CD | GLU | B | 222 | −19.162 | −16.024 | −32.990 | 1.00 | 41.59 | 6 | C |
| ATOM | 3433 | OE1 | GLU | B | 222 | −18.071 | −15.787 | −32.426 | 1.00 | 42.25 | 8 | O |
| ATOM | 3434 | OE2 | GLU | B | 222 | −19.245 | −16.296 | −34.220 | 1.00 | 44.47 | 8 | O |
| ATOM | 3435 | C | GLU | B | 222 | −22.222 | −12.758 | −31.725 | 1.00 | 30.78 | 6 | C |
| ATOM | 3436 | O | GLU | B | 222 | −21.904 | −12.116 | −30.715 | 1.00 | 30.00 | 8 | O |
| ATOM | 3437 | N | LEU | B | 223 | −22.388 | −12.189 | −32.913 | 1.00 | 29.53 | 7 | N |
| ATOM | 3438 | CA | LEU | B | 223 | −22.209 | −10.757 | −33.092 | 1.00 | 28.77 | 6 | C |
| ATOM | 3439 | CB | LEU | B | 223 | −22.097 | −10.417 | −34.586 | 1.00 | 29.52 | 6 | C |
| ATOM | 3440 | CG | LEU | B | 223 | −21.901 | −8.933 | −34.861 | 1.00 | 30.50 | 6 | C |
| ATOM | 3441 | CD1 | LEU | B | 223 | −20.755 | −8.407 | −34.013 | 1.00 | 31.04 | 6 | C |
| ATOM | 3442 | CD2 | LEU | B | 223 | −21.651 | −8.685 | −36.358 | 1.00 | 32.50 | 6 | C |
| ATOM | 3443 | C | LEU | B | 223 | −23.330 | −9.950 | −32.453 | 1.00 | 28.10 | 6 | C |
| ATOM | 3444 | O | LEU | B | 223 | −23.075 | −8.936 | −31.809 | 1.00 | 27.14 | 8 | O |
| ATOM | 3445 | N | ALA | B | 224 | −24.574 | −10.394 | −32.631 | 1.00 | 27.70 | 7 | N |
| ATOM | 3446 | CA | ALA | B | 224 | −25.721 | −9.676 | −32.070 | 1.00 | 26.94 | 6 | C |
| ATOM | 3447 | CB | ALA | B | 224 | −27.044 | −10.393 | −32.420 | 1.00 | 27.22 | 6 | C |
| ATOM | 3448 | C | ALA | B | 224 | −25.609 | −9.496 | −30.556 | 1.00 | 27.06 | 6 | C |
| ATOM | 3449 | O | ALA | B | 224 | −25.849 | −8.408 | −30.034 | 1.00 | 26.24 | 8 | O |
| ATOM | 3450 | N | SER | B | 225 | −25.269 | −10.575 | −29.854 | 1.00 | 26.87 | 7 | N |
| ATOM | 3451 | CA | SER | B | 225 | −25.155 | −10.529 | −28.399 | 1.00 | 27.50 | 6 | C |
| ATOM | 3452 | CB | SER | B | 225 | −24.989 | −11.945 | −27.823 | 1.00 | 27.52 | 6 | C |
| ATOM | 3453 | OG | SER | B | 225 | −23.738 | −12.509 | −28.196 | 1.00 | 29.93 | 8 | O |
| ATOM | 3454 | C | SER | B | 225 | −24.023 | −9.587 | −27.964 | 1.00 | 27.29 | 6 | C |
| ATOM | 3455 | O | SER | B | 225 | −24.152 | −8.853 | −26.979 | 1.00 | 27.90 | 8 | O |
| ATOM | 3456 | N | ARG | B | 226 | −22.929 | −9.563 | −28.712 | 1.00 | 26.68 | 7 | N |
| ATOM | 3457 | CA | ARG | B | 226 | −21.837 | −8.660 | −28.355 | 1.00 | 26.54 | 6 | C |
| ATOM | 3458 | CB | ARG | B | 226 | −20.537 | −9.070 | −29.038 | 1.00 | 26.73 | 6 | C |
| ATOM | 3459 | CG | ARG | B | 226 | −19.945 | −10.333 | −28.437 | 1.00 | 26.92 | 6 | C |
| ATOM | 3460 | CD | ARG | B | 226 | −18.949 | −11.028 | −29.331 | 1.00 | 28.63 | 6 | C |
| ATOM | 3461 | NE | ARG | B | 226 | −18.380 | −12.205 | −28.675 | 1.00 | 28.84 | 7 | N |
| ATOM | 3462 | CZ | ARG | B | 226 | −19.015 | −13.364 | −28.558 | 1.00 | 30.18 | 6 | C |
| ATOM | 3463 | NH1 | ARG | B | 226 | −20.231 | −13.511 | −29.066 | 1.00 | 29.81 | 7 | N |
| ATOM | 3464 | NH2 | ARG | B | 226 | −18.428 | −14.386 | −27.938 | 1.00 | 30.49 | 7 | N |
| ATOM | 3465 | C | ARG | B | 226 | −22.171 | −7.188 | −28.621 | 1.00 | 26.70 | 6 | C |
| ATOM | 3466 | O | ARG | B | 226 | −21.670 | −6.307 | −27.917 | 1.00 | 26.13 | 8 | O |
| ATOM | 3467 | N | LEU | B | 227 | −23.018 | −6.932 | −29.625 | 1.00 | 26.74 | 7 | N |
| ATOM | 3468 | CA | LEU | B | 227 | −23.500 | −5.573 | −29.911 | 1.00 | 27.07 | 6 | C |
| ATOM | 3469 | CB | LEU | B | 227 | −24.206 | −5.510 | −31.277 | 1.00 | 27.06 | 6 | C |
| ATOM | 3470 | CG | LEU | B | 227 | −23.289 | −5.649 | −32.505 | 1.00 | 27.87 | 6 | C |
| ATOM | 3471 | CD1 | LEU | B | 227 | −24.049 | −5.763 | −33.847 | 1.00 | 29.73 | 6 | C |
| ATOM | 3472 | CD2 | LEU | B | 227 | −22.303 | −4.492 | −32.555 | 1.00 | 28.95 | 6 | C |
| ATOM | 3473 | C | LEU | B | 227 | −24.424 | −5.054 | −28.799 | 1.00 | 27.49 | 6 | C |
| ATOM | 3474 | O | LEU | B | 227 | −24.465 | −3.852 | −28.523 | 1.00 | 27.09 | 8 | O |
| ATOM | 3475 | N | ARG | B | 228 | −25.183 | −5.960 | −28.178 | 1.00 | 27.37 | 7 | N |
| ATOM | 3476 | CA | ARG | B | 228 | −26.016 | −5.593 | −27.045 | 1.00 | 27.38 | 6 | C |
| ATOM | 3477 | CB | ARG | B | 228 | −26.915 | −6.751 | −26.610 | 1.00 | 28.17 | 6 | C |
| ATOM | 3478 | CG | ARG | B | 228 | −28.199 | −6.972 | −27.404 | 1.00 | 28.52 | 6 | C |
| ATOM | 3479 | CD | ARG | B | 228 | −29.157 | −7.933 | −26.667 | 1.00 | 33.60 | 6 | C |
| ATOM | 3480 | NE | ARG | B | 228 | −28.917 | −9.320 | −27.044 | 1.00 | 36.16 | 7 | N |
| ATOM | 3481 | CZ | ARG | B | 228 | −28.486 | −10.282 | −26.252 | 1.00 | 38.65 | 6 | C |
| ATOM | 3482 | NH1 | ARG | B | 228 | −28.239 | −10.052 | −24.961 | 1.00 | 42.93 | 7 | N |
| ATOM | 3483 | NH2 | ARG | B | 228 | −28.316 | −11.500 | −26.753 | 1.00 | 36.19 | 7 | N |
| ATOM | 3484 | C | ARG | B | 228 | −25.111 | −5.184 | −25.875 | 1.00 | 27.29 | 6 | C |
| ATOM | 3485 | O | ARG | B | 228 | −25.394 | −4.213 | −25.187 | 1.00 | 26.61 | 8 | O |
| ATOM | 3486 | N | TYR | B | 229 | −24.031 | −5.932 | −25.643 | 1.00 | 26.96 | 7 | N |
| ATOM | 3487 | CA | TYR | B | 229 | −23.086 | −5.588 | −24.573 | 1.00 | 27.38 | 6 | C |
| ATOM | 3488 | CB | TYR | B | 229 | −22.025 | −6.686 | −24.396 | 1.00 | 27.60 | 6 | C |
| ATOM | 3489 | CG | TYR | B | 229 | −21.122 | −6.500 | −23.184 | 1.00 | 28.68 | 6 | C |
| ATOM | 3490 | CD1 | TYR | B | 229 | −21.572 | −6.796 | −21.909 | 1.00 | 30.32 | 6 | C |
| ATOM | 3491 | CE1 | TYR | B | 229 | −20.760 | −6.625 | −20.798 | 1.00 | 30.49 | 6 | C |
| ATOM | 3492 | CZ | TYR | B | 229 | −19.479 | −6.162 | −20.961 | 1.00 | 32.12 | 6 | C |
| ATOM | 3493 | OH | TYR | B | 229 | −18.669 | −5.989 | −19.858 | 1.00 | 33.30 | 8 | O |
| ATOM | 3494 | CE2 | TYR | B | 229 | −19.007 | −5.850 | −22.218 | 1.00 | 31.11 | 6 | C |
| ATOM | 3495 | CD2 | TYR | B | 229 | −19.832 | −6.017 | −23.321 | 1.00 | 30.01 | 6 | C |
| ATOM | 3496 | C | TYR | B | 229 | −22.410 | −4.261 | −24.906 | 1.00 | 26.96 | 6 | C |
| ATOM | 3497 | O | TYR | B | 229 | −22.198 | −3.409 | −24.031 | 1.00 | 27.00 | 8 | O |
| ATOM | 3498 | N | ALA | B | 230 | −22.070 | −4.093 | −26.178 | 1.00 | 26.19 | 7 | N |
| ATOM | 3499 | CA | ALA | B | 230 | −21.397 | −2.879 | −26.612 | 1.00 | 26.77 | 6 | C |
| ATOM | 3500 | CB | ALA | B | 230 | −21.096 | −2.930 | −28.094 | 1.00 | 25.91 | 6 | C |
| ATOM | 3501 | C | ALA | B | 230 | −22.232 | −1.651 | −26.285 | 1.00 | 26.84 | 6 | C |
| ATOM | 3502 | O | ALA | B | 230 | −21.705 | −0.652 | −25.830 | 1.00 | 27.61 | 8 | O |
| ATOM | 3503 | N | ARG | B | 231 | −23.533 | −1.723 | −26.523 | 1.00 | 27.30 | 7 | N |
| ATOM | 3504 | CA | ARG | B | 231 | −24.383 | −0.574 | −26.241 | 1.00 | 27.93 | 6 | C |
| ATOM | 3505 | CB | ARG | B | 231 | −25.831 | −0.838 | −26.671 | 1.00 | 27.44 | 6 | C |
| ATOM | 3506 | CG | ARG | B | 231 | −26.767 | 0.364 | −26.489 | 1.00 | 28.31 | 6 | C |
| ATOM | 3507 | CD | ARG | B | 231 | −27.516 | 0.376 | −25.153 | 1.00 | 29.17 | 6 | C |
| ATOM | 3508 | NE | ARG | B | 231 | −28.261 | 1.625 | −24.958 | 1.00 | 31.21 | 7 | N |
| ATOM | 3509 | CZ | ARG | B | 231 | −29.409 | 1.917 | −25.571 | 1.00 | 32.23 | 6 | C |
| ATOM | 3510 | NH1 | ARG | B | 231 | −29.949 | 1.054 | −26.419 | 1.00 | 32.15 | 7 | N |
| ATOM | 3511 | NH2 | ARG | B | 231 | −30.017 | 3.079 | −25.343 | 1.00 | 32.54 | 7 | N |
| ATOM | 3512 | C | ARG | B | 231 | −24.293 | −0.226 | −24.756 | 1.00 | 28.42 | 6 | C |
| ATOM | 3513 | O | ARG | B | 231 | −24.230 | 0.953 | −24.382 | 1.00 | 28.53 | 8 | O |
| ATOM | 3514 | N | THR | B | 232 | −24.291 | −1.249 | −23.903 | 1.00 | 28.73 | 7 | N |
| ATOM | 3515 | CA | THR | B | 232 | −24.150 | −1.006 | −22.466 | 1.00 | 29.42 | 6 | C |
| ATOM | 3516 | CB | THR | B | 232 | −24.227 | −2.319 | −21.670 | 1.00 | 29.47 | 6 | C |
| ATOM | 3517 | OG1 | THR | B | 232 | −25.451 | −2.985 | −21.987 | 1.00 | 29.28 | 8 | O |
| ATOM | 3518 | CG2 | THR | B | 232 | −24.353 | −2.026 | −20.173 | 1.00 | 30.08 | 6 | C |
| ATOM | 3519 | C | THR | B | 232 | −22.855 | −0.264 | −22.141 | 1.00 | 29.68 | 6 | C |
| ATOM | 3520 | O | THR | B | 232 | −22.860 | 0.682 | −21.341 | 1.00 | 29.95 | 8 | O |
| ATOM | 3521 | N | MET | B | 233 | −21.754 | −0.687 | −22.762 | 1.00 | 30.00 | 7 | N |
| ATOM | 3522 | CA | MET | B | 233 | −20.441 | −0.069 | −22.544 | 1.00 | 30.54 | 6 | C |
| ATOM | 3523 | CB | MET | B | 233 | −19.337 | −0.872 | −23.245 | 1.00 | 30.12 | 6 | C |
| ATOM | 3524 | CG | MET | B | 233 | −19.145 | −2.306 | −22.732 | 1.00 | 30.73 | 6 | C |
| ATOM | 3525 | SD | MET | B | 233 | −18.887 | −2.407 | −20.932 | 1.00 | 31.99 | 16 | S |
| ATOM | 3526 | CE | MET | B | 233 | −20.446 | −2.980 | −20.352 | 1.00 | 26.77 | 6 | C |
| ATOM | 3527 | C | MET | B | 233 | −20.402 | 1.387 | −23.018 | 1.00 | 31.10 | 6 | C |
| ATOM | 3528 | O | MET | B | 233 | −19.817 | 2.257 | −22.364 | 1.00 | 30.77 | 8 | O |
| ATOM | 3529 | N | VAL | B | 234 | −21.016 | 1.647 | −24.166 | 1.00 | 31.50 | 7 | N |
| ATOM | 3530 | CA | VAL | B | 234 | −21.057 | 3.010 | −24.691 | 1.00 | 32.56 | 6 | C |
| ATOM | 3531 | CB | VAL | B | 234 | −21.588 | 3.031 | −26.133 | 1.00 | 32.17 | 6 | C |
| ATOM | 3532 | CG1 | VAL | B | 234 | −21.888 | 4.459 | −26.576 | 1.00 | 32.60 | 6 | C |
| ATOM | 3533 | CG2 | VAL | B | 234 | −20.567 | 2.359 | −27.047 | 1.00 | 31.40 | 6 | C |
| ATOM | 3534 | C | VAL | B | 234 | −21.859 | 3.927 | −23.760 | 1.00 | 33.51 | 6 | C |
| ATOM | 3535 | O | VAL | B | 234 | −21.476 | 5.082 | −23.522 | 1.00 | 33.37 | 8 | O |
| ATOM | 3536 | N | ASP | B | 235 | −22.952 | 3.403 | −23.211 | 1.00 | 34.93 | 7 | N |
| ATOM | 3537 | CA | ASP | B | 235 | −23.728 | 4.142 | −22.225 | 1.00 | 36.74 | 6 | C |
| ATOM | 3538 | CB | ASP | B | 235 | −24.939 | 3.335 | −21.758 | 1.00 | 37.07 | 6 | C |
| ATOM | 3539 | CG | ASP | B | 235 | −26.181 | 3.614 | −22.582 | 1.00 | 38.43 | 6 | C |
| ATOM | 3540 | OD1 | ASP | B | 235 | −26.322 | 4.750 | −23.090 | 1.00 | 39.46 | 8 | O |
| ATOM | 3541 | OD2 | ASP | B | 235 | −27.080 | 2.768 | −22.764 | 1.00 | 40.23 | 8 | O |
| ATOM | 3542 | C | ASP | B | 235 | −22.845 | 4.503 | −21.028 | 1.00 | 37.73 | 6 | C |
| ATOM | 3543 | O | ASP | B | 235 | −22.965 | 5.594 | −20.473 | 1.00 | 38.05 | 8 | O |
| ATOM | 3544 | N | LYS | B | 236 | −21.963 | 3.585 | −20.630 | 1.00 | 38.45 | 7 | N |
| ATOM | 3545 | CA | LYS | B | 236 | −21.037 | 3.841 | −19.525 | 1.00 | 39.37 | 6 | C |
| ATOM | 3546 | CB | LYS | B | 236 | −20.323 | 2.551 | −19.090 | 1.00 | 39.23 | 6 | C |
| ATOM | 3547 | CG | LYS | B | 236 | −21.217 | 1.548 | −18.366 | 1.00 | 39.71 | 6 | C |
| ATOM | 3548 | CD | LYS | B | 236 | −20.471 | 0.246 | −18.082 | 1.00 | 41.45 | 6 | C |
| ATOM | 3549 | CE | LYS | B | 236 | −21.307 | −0.722 | −17.248 | 1.00 | 42.36 | 6 | C |
| ATOM | 3550 | NZ | LYS | B | 236 | −21.538 | −0.227 | −15.857 | 1.00 | 43.08 | 7 | N |
| ATOM | 3551 | C | LYS | B | 236 | −20.023 | 4.945 | −19.872 | 1.00 | 40.08 | 6 | C |
| ATOM | 3552 | O | LYS | B | 236 | −19.752 | 5.822 | −19.049 | 1.00 | 40.28 | 8 | O |
| ATOM | 3553 | N | LEU | B | 237 | −19.472 | 4.909 | −21.083 | 1.00 | 41.07 | 7 | N |
| ATOM | 3554 | CA | LEU | B | 237 | −18.556 | 5.959 | −21.531 | 1.00 | 42.34 | 6 | C |
| ATOM | 3555 | CB | LEU | B | 237 | −18.019 | 5.659 | −22.930 | 1.00 | 41.72 | 6 | C |
| ATOM | 3556 | CG | LEU | B | 237 | −17.053 | 4.480 | −23.116 | 1.00 | 41.42 | 6 | C |
| ATOM | 3557 | CD1 | LEU | B | 237 | −16.740 | 4.272 | −24.589 | 1.00 | 40.13 | 6 | C |
| ATOM | 3558 | CD2 | LEU | B | 237 | −15.768 | 4.706 | −22.325 | 1.00 | 40.29 | 6 | C |
| ATOM | 3559 | C | LEU | B | 237 | −19.270 | 7.315 | −21.526 | 1.00 | 43.87 | 6 | C |
| ATOM | 3560 | O | LEU | B | 237 | −18.680 | 8.346 | −21.188 | 1.00 | 43.65 | 8 | O |
| ATOM | 3561 | N | LEU | B | 238 | −20.543 | 7.303 | −21.909 | 1.00 | 45.67 | 7 | N |
| ATOM | 3562 | CA | LEU | B | 238 | −21.360 | 8.516 | −21.938 | 1.00 | 47.83 | 6 | C |
| ATOM | 3563 | CB | LEU | B | 238 | −22.634 | 8.268 | −22.739 | 1.00 | 47.56 | 6 | C |
| ATOM | 3564 | CG | LEU | B | 238 | −22.472 | 8.426 | −24.246 | 1.00 | 48.06 | 6 | C |
| ATOM | 3565 | CD1 | LEU | B | 238 | −23.576 | 7.688 | −24.991 | 1.00 | 48.48 | 6 | C |
| ATOM | 3566 | CD2 | LEU | B | 238 | −22.452 | 9.905 | −24.616 | 1.00 | 47.93 | 6 | C |
| ATOM | 3567 | C | LEU | B | 238 | −21.725 | 9.030 | −20.549 | 1.00 | 49.45 | 6 | C |
| ATOM | 3568 | O | LEU | B | 238 | −21.847 | 10.240 | −20.335 | 1.00 | 49.72 | 8 | O |
| ATOM | 3569 | N | SER | B | 239 | −21.908 | 8.110 | −19.609 | 1.00 | 51.38 | 7 | N |
| ATOM | 3570 | CA | SER | B | 239 | −22.268 | 8.477 | −18.245 | 1.00 | 53.19 | 6 | C |
| ATOM | 3571 | CB | SER | B | 239 | −22.356 | 7.233 | −17.361 | 1.00 | 53.24 | 6 | C |
| ATOM | 3572 | OG | SER | B | 239 | −21.057 | 6.755 | −17.034 | 1.00 | 54.23 | 8 | O |
| ATOM | 3573 | C | SER | B | 239 | −21.252 | 9.437 | −17.649 | 1.00 | 54.22 | 6 | C |
| ATOM | 3574 | O | SER | B | 239 | −21.596 | 10.537 | −17.217 | 1.00 | 54.39 | 8 | O |
| ATOM | 3575 | N | SER | B | 240 | −19.996 | 9.006 | −17.635 | 1.00 | 55.60 | 7 | N |
| ATOM | 3576 | CA | SER | B | 240 | −18.910 | 9.784 | −17.054 | 1.00 | 56.85 | 6 | C |
| ATOM | 3577 | CB | SER | B | 240 | −18.060 | 8.884 | −16.162 | 1.00 | 56.87 | 6 | C |
| ATOM | 3578 | OG | SER | B | 240 | −17.359 | 7.932 | −16.948 | 1.00 | 57.47 | 8 | O |
| ATOM | 3579 | C | SER | B | 240 | −18.017 | 10.383 | −18.128 | 1.00 | 57.56 | 6 | C |
| ATOM | 3580 | O | SER | B | 240 | −16.807 | 10.141 | −18.132 | 1.00 | 58.04 | 8 | O |
| ATOM | 3581 | N | ALA | B | 241 | −18.605 | 11.155 | −19.039 | 1.00 | 58.16 | 7 | N |
| ATOM | 3582 | CA | ALA | B | 241 | −17.842 | 11.764 | −20.125 | 1.00 | 58.82 | 6 | C |
| ATOM | 3583 | CB | ALA | B | 241 | −18.769 | 12.239 | −21.236 | 1.00 | 58.78 | 6 | C |
| ATOM | 3584 | C | ALA | B | 241 | −16.964 | 12.912 | −19.633 | 1.00 | 59.25 | 6 | C |
| ATOM | 3585 | O | ALA | B | 241 | −17.366 | 14.082 | −19.667 | 1.00 | 59.83 | 8 | O |
| ATOM | 3586 | OXT | ALA | B | 241 | −15.832 | 12.686 | −19.195 | 1.00 | 59.46 | 8 | O |
| ATOM | 3587 | N | PRO | E | 1 | 16.379 | −7.591 | 9.788 | 1.00 | 40.91 | 7 | N |
| ATOM | 3588 | CA | PRO | E | 1 | 15.544 | −7.800 | 8.572 | 1.00 | 40.69 | 6 | C |
| ATOM | 3589 | CB | PRO | E | 1 | 14.852 | −6.442 | 8.381 | 1.00 | 40.92 | 6 | C |
| ATOM | 3590 | CG | PRO | E | 1 | 15.200 | −5.629 | 9.591 | 1.00 | 41.09 | 6 | C |
| ATOM | 3591 | CD | PRO | E | 1 | 16.488 | −6.166 | 10.134 | 1.00 | 41.02 | 6 | C |
| ATOM | 3592 | C | PRO | E | 1 | 16.423 | −8.073 | 7.359 | 1.00 | 40.59 | 6 | C |
| ATOM | 3593 | O | PRO | E | 1 | 17.539 | −7.559 | 7.287 | 1.00 | 40.41 | 8 | O |
| ATOM | 3594 | N | MET | E | 2 | 15.918 | −8.856 | 6.411 | 1.00 | 40.05 | 7 | N |
| ATOM | 3595 | CA | MET | E | 2 | 16.683 | −9.168 | 5.215 | 1.00 | 40.01 | 6 | C |
| ATOM | 3596 | CB | MET | E | 2 | 16.476 | −10.634 | 4.812 | 1.00 | 41.09 | 6 | C |
| ATOM | 3597 | CG | MET | E | 2 | 17.149 | −11.615 | 5.768 | 1.00 | 43.73 | 6 | C |
| ATOM | 3598 | SD | MET | E | 2 | 18.945 | −11.390 | 5.805 | 1.00 | 51.91 | 16 | S |
| ATOM | 3599 | CE | MET | E | 2 | 19.424 | −12.458 | 7.142 | 1.00 | 52.26 | 6 | C |
| ATOM | 3600 | C | MET | E | 2 | 16.326 | −8.218 | 4.083 | 1.00 | 38.83 | 6 | C |
| ATOM | 3601 | O | MET | E | 2 | 16.804 | −8.365 | 2.957 | 1.00 | 38.60 | 8 | O |
| ATOM | 3602 | N | GLN | E | 3 | 15.486 | −7.233 | 4.394 | 1.00 | 37.38 | 7 | N |
| ATOM | 3603 | CA | GLN | E | 3 | 15.069 | −6.232 | 3.427 | 1.00 | 36.61 | 6 | C |
| ATOM | 3604 | CB | GLN | E | 3 | 13.811 | −6.680 | 2.676 | 1.00 | 37.52 | 6 | C |
| ATOM | 3605 | CG | GLN | E | 3 | 12.608 | −7.020 | 3.564 | 1.00 | 39.75 | 6 | C |
| ATOM | 3606 | CD | GLN | E | 3 | 11.396 | −7.467 | 2.757 | 1.00 | 44.85 | 6 | C |
| ATOM | 3607 | OE1 | GLN | E | 3 | 11.509 | −7.723 | 1.556 | 1.00 | 46.70 | 8 | O |
| ATOM | 3608 | NE2 | GLN | E | 3 | 10.235 | −7.553 | 3.411 | 1.00 | 46.10 | 7 | N |
| ATOM | 3609 | C | GLN | E | 3 | 14.793 | −4.932 | 4.163 | 1.00 | 35.58 | 6 | C |
| ATOM | 3610 | O | GLN | E | 3 | 14.584 | −4.938 | 5.377 | 1.00 | 34.86 | 8 | O |
| ATOM | 3611 | N | SER | E | 4 | 14.809 | −3.821 | 3.437 | 1.00 | 34.23 | 7 | N |
| ATOM | 3612 | CA | SER | E | 4 | 14.508 | −2.524 | 4.040 | 1.00 | 33.40 | 6 | C |
| ATOM | 3613 | CB | SER | E | 4 | 14.962 | −1.393 | 3.125 | 1.00 | 32.85 | 6 | C |
| ATOM | 3614 | OG | SER | E | 4 | 14.259 | −1.436 | 1.895 | 1.00 | 31.74 | 8 | O |
| ATOM | 3615 | C | SER | E | 4 | 13.003 | −2.416 | 4.303 | 1.00 | 33.74 | 6 | C |
| ATOM | 3616 | O | SER | E | 4 | 12.262 | −3.389 | 4.107 | 1.00 | 33.39 | 8 | O |
| ATOM | 3617 | O3P | TPO | E | 5 | 13.899 | 1.162 | 8.266 | 1.00 | 30.51 | 8 | O |
| ATOM | 3618 | P | TPO | E | 5 | 13.192 | 1.401 | 6.861 | 1.00 | 32.40 | 15 | P |
| ATOM | 3619 | O1P | TPO | E | 5 | 12.542 | 2.823 | 6.637 | 1.00 | 32.45 | 8 | O |
| ATOM | 3620 | O2P | TPO | E | 5 | 14.048 | 0.875 | 5.613 | 1.00 | 31.07 | 8 | O |
| ATOM | 3621 | OG1 | TPO | E | 5 | 11.927 | 0.412 | 6.990 | 1.00 | 32.05 | 8 | O |
| ATOM | 3622 | CB | TPO | E | 5 | 11.038 | 0.274 | 5.883 | 1.00 | 33.24 | 6 | C |
| ATOM | 3623 | CG2 | TPO | E | 5 | 9.631 | 0.611 | 6.355 | 1.00 | 34.92 | 6 | C |
| ATOM | 3624 | CA | TPO | E | 5 | 11.111 | −1.171 | 5.347 | 1.00 | 33.88 | 6 | C |
| ATOM | 3625 | N | TPO | E | 5 | 12.470 | −1.314 | 4.833 | 1.00 | 33.34 | 7 | N |
| ATOM | 3626 | C | TPO | E | 5 | 10.057 | −1.420 | 4.285 | 1.00 | 34.38 | 6 | C |
| ATOM | 3627 | O | TPO | E | 5 | 10.147 | −0.852 | 3.087 | 1.00 | 33.69 | 8 | O |
| ATOM | 3628 | N | PRO | E | 6 | 9.130 | −2.342 | 4.537 | 1.00 | 38.71 | 7 | N |
| ATOM | 3629 | CA | PRO | E | 6 | 8.008 | −2.757 | 3.643 | 1.00 | 40.41 | 6 | C |
| ATOM | 3630 | CB | PRO | E | 6 | 7.331 | −3.894 | 4.422 | 1.00 | 40.16 | 6 | C |
| ATOM | 3631 | CG | PRO | E | 6 | 8.323 | −4.323 | 5.457 | 1.00 | 40.63 | 6 | C |
| ATOM | 3632 | CD | PRO | E | 6 | 9.129 | −3.091 | 5.804 | 1.00 | 39.21 | 6 | C |
| ATOM | 3633 | C | PRO | E | 6 | 6.999 | −1.642 | 3.392 | 1.00 | 41.29 | 6 | C |
| ATOM | 3634 | O | PRO | E | 6 | 6.811 | −0.739 | 4.215 | 1.00 | 41.42 | 8 | O |
| ATOM | 3635 | N | LEU | E | 7 | 6.338 | −1.742 | 2.247 | 1.00 | 42.62 | 7 | N |
| ATOM | 3636 | CA | LEU | E | 7 | 5.340 | −0.786 | 1.797 | 1.00 | 43.90 | 6 | C |
| ATOM | 3637 | CB | LEU | E | 7 | 4.866 | −1.200 | 0.403 | 1.00 | 44.21 | 6 | C |
| ATOM | 3638 | CG | LEU | E | 7 | 4.188 | −0.160 | −0.479 | 1.00 | 45.75 | 6 | C |
| ATOM | 3639 | CD1 | LEU | E | 7 | 4.942 | 1.161 | −0.420 | 1.00 | 46.28 | 6 | C |
| ATOM | 3640 | CD2 | LEU | E | 7 | 4.097 | −0.682 | −1.911 | 1.00 | 46.71 | 6 | C |
| ATOM | 3641 | C | LEU | E | 7 | 4.152 | −0.676 | 2.758 | 1.00 | 44.39 | 6 | C |
| ATOM | 3642 | O | LEU | E | 7 | 3.923 | −1.564 | 3.592 | 1.00 | 45.42 | 8 | O |
| ATOM | 3643 | N | PRO | F | 1 | −7.373 | −9.873 | −15.860 | 1.00 | 63.61 | 7 | N |
| ATOM | 3644 | CA | PRO | F | 1 | −6.089 | −9.838 | −16.612 | 1.00 | 63.48 | 6 | C |
| ATOM | 3645 | CB | PRO | F | 1 | −6.509 | −10.235 | −18.031 | 1.00 | 63.69 | 6 | C |
| ATOM | 3646 | CG | PRO | F | 1 | −7.815 | −10.957 | −17.863 | 1.00 | 63.62 | 6 | C |
| ATOM | 3647 | CD | PRO | F | 1 | −8.500 | −10.293 | −16.711 | 1.00 | 63.72 | 6 | C |
| ATOM | 3648 | C | PRO | F | 1 | −5.498 | −8.433 | −16.617 | 1.00 | 63.40 | 6 | C |
| ATOM | 3649 | O | PRO | F | 1 | −6.217 | −7.470 | −16.871 | 1.00 | 63.50 | 8 | O |
| ATOM | 3650 | N | MET | F | 2 | −4.204 | −8.315 | −16.343 | 1.00 | 63.09 | 7 | N |
| ATOM | 3651 | CA | MET | F | 2 | −3.557 | −7.008 | −16.314 | 1.00 | 62.84 | 6 | C |
| ATOM | 3652 | CB | MET | F | 2 | −2.636 | −6.893 | −15.099 | 1.00 | 63.11 | 6 | C |
| ATOM | 3653 | CG | MET | F | 2 | −3.383 | −6.837 | −13.779 | 1.00 | 64.18 | 6 | C |
| ATOM | 3654 | SD | MET | F | 2 | −4.393 | −5.349 | −13.628 | 1.00 | 66.44 | 16 | S |
| ATOM | 3655 | CE | MET | F | 2 | −5.583 | −5.865 | −12.403 | 1.00 | 66.00 | 6 | C |
| ATOM | 3656 | C | MET | F | 2 | −2.780 | −6.745 | −17.594 | 1.00 | 62.32 | 6 | C |
| ATOM | 3657 | O | MET | F | 2 | −2.021 | −5.781 | −17.685 | 1.00 | 62.29 | 8 | O |
| ATOM | 3658 | N | GLN | F | 3 | −2.984 | −7.603 | −18.585 | 1.00 | 61.72 | 7 | N |
| ATOM | 3659 | CA | GLN | F | 3 | −2.304 | −7.475 | −19.864 | 1.00 | 61.27 | 6 | C |
| ATOM | 3660 | CB | GLN | F | 3 | −0.896 | −8.057 | −19.762 | 1.00 | 61.46 | 6 | C |
| ATOM | 3661 | CG | GLN | F | 3 | −0.859 | −9.414 | −19.086 | 1.00 | 62.29 | 6 | C |
| ATOM | 3662 | CD | GLN | F | 3 | 0.506 | −10.055 | −19.147 | 1.00 | 63.90 | 6 | C |
| ATOM | 3663 | OE1 | GLN | F | 3 | 1.511 | −9.373 | −19.352 | 1.00 | 64.45 | 8 | O |
| ATOM | 3664 | NE2 | GLN | F | 3 | 0.550 | −11.371 | −18.974 | 1.00 | 64.33 | 7 | N |
| ATOM | 3665 | C | GLN | F | 3 | −3.078 | −8.213 | −20.951 | 1.00 | 60.64 | 6 | C |
| ATOM | 3666 | O | GLN | F | 3 | −3.908 | −9.072 | −20.661 | 1.00 | 60.37 | 8 | O |
| ATOM | 3667 | N | SER | F | 4 | −2.802 | −7.874 | −22.204 | 1.00 | 60.11 | 7 | N |
| ATOM | 3668 | CA | SER | F | 4 | −3.463 | −8.531 | −23.324 | 1.00 | 59.74 | 6 | C |
| ATOM | 3669 | CB | SER | F | 4 | −3.501 | −7.620 | −24.550 | 1.00 | 59.65 | 6 | C |
| ATOM | 3670 | OG | SER | F | 4 | −2.201 | −7.299 | −25.014 | 1.00 | 59.69 | 8 | O |
| ATOM | 3671 | C | SER | F | 4 | −2.765 | −9.846 | −23.654 | 1.00 | 59.29 | 6 | C |
| ATOM | 3672 | O | SER | F | 4 | −2.174 | −10.478 | −22.776 | 1.00 | 59.28 | 8 | O |
| ATOM | 3673 | O3P | TPO | F | 5 | −6.281 | −11.938 | −27.798 | 1.00 | 52.19 | 8 | O |
| ATOM | 3674 | P | TPO | F | 5 | −6.257 | −11.464 | −26.261 | 1.00 | 51.54 | 15 | P |
| ATOM | 3675 | O1P | TPO | F | 5 | −5.611 | −10.011 | −26.100 | 1.00 | 51.18 | 8 | O |
| ATOM | 3676 | O2P | TPO | F | 5 | −7.603 | −11.821 | −25.481 | 1.00 | 49.40 | 8 | O |
| ATOM | 3677 | OG1 | TPO | F | 5 | −5.200 | −12.467 | −25.572 | 1.00 | 55.49 | 8 | O |
| ATOM | 3678 | CB | TPO | F | 5 | −3.824 | −12.505 | −25.934 | 1.00 | 57.12 | 6 | C |
| ATOM | 3679 | CG2 | TPO | F | 5 | −3.469 | −13.923 | −26.369 | 1.00 | 57.14 | 6 | C |
| ATOM | 3680 | CA | TPO | F | 5 | −2.991 | −12.082 | −24.729 | 1.00 | 57.42 | 6 | C |
| ATOM | 3681 | N | TPO | F | 5 | −3.256 | −10.658 | −24.584 | 1.00 | 58.07 | 7 | N |
| ATOM | 3682 | C | TPO | F | 5 | −1.523 | −12.356 | −24.980 | 1.00 | 58.27 | 6 | C |
| ATOM | 3683 | O | TPO | F | 5 | −0.801 | −11.544 | −25.752 | 1.00 | 57.29 | 8 | O |
| ATOM | 3684 | N | PRO | F | 6 | −1.153 | −13.293 | −24.105 | 1.00 | 61.36 | 7 | N |
| ATOM | 3685 | CA | PRO | F | 6 | 0.332 | −13.349 | −24.226 | 1.00 | 62.33 | 6 | C |
| ATOM | 3686 | CB | PRO | F | 6 | 0.746 | −14.060 | −22.935 | 1.00 | 62.12 | 6 | C |
| ATOM | 3687 | CG | PRO | F | 6 | −0.506 | −14.752 | −22.491 | 1.00 | 62.02 | 6 | C |
| ATOM | 3688 | CD | PRO | F | 6 | −1.596 | −13.761 | −22.781 | 1.00 | 61.75 | 6 | C |
| ATOM | 3689 | C | PRO | F | 6 | 0.781 | −14.173 | −25.426 | 1.00 | 62.85 | 6 | C |
| ATOM | 3690 | O | PRO | F | 6 | 0.027 | −15.003 | −25.931 | 1.00 | 63.05 | 8 | O |
| ATOM | 3691 | N | LEU | F | 7 | 2.012 | −13.945 | −25.866 | 1.00 | 63.66 | 7 | N |
| ATOM | 3692 | CA | LEU | F | 7 | 2.579 | −14.672 | −26.994 | 1.00 | 64.38 | 6 | C |
| ATOM | 3693 | CB | LEU | F | 7 | 3.904 | −14.039 | −27.415 | 1.00 | 64.68 | 6 | C |
| ATOM | 3694 | CG | LEU | F | 7 | 4.503 | −14.555 | −28.720 | 1.00 | 65.80 | 6 | C |
| ATOM | 3695 | CD1 | LEU | F | 7 | 3.411 | −14.781 | −29.760 | 1.00 | 66.75 | 6 | C |
| ATOM | 3696 | CD2 | LEU | F | 7 | 5.559 | −13.587 | −29.234 | 1.00 | 66.83 | 6 | C |
| ATOM | 3697 | C | LEU | F | 7 | 2.786 | −16.146 | −26.663 | 1.00 | 64.48 | 6 | C |
| ATOM | 3698 | O | LEU | F | 7 | 2.810 | −16.535 | −25.493 | 1.00 | 64.81 | 8 | O |
| ATOM | 3699 | O | WAT | W | 1 | 24.634 | 2.439 | −7.629 | 1.00 | 30.47 | 8 | |
| ATOM | 3700 | O | WAT | W | 2 | 17.166 | 2.736 | 2.573 | 1.00 | 32.38 | 8 | |
| ATOM | 3701 | O | WAT | W | 3 | 27.595 | 14.681 | 23.241 | 1.00 | 29.83 | 8 | |
| ATOM | 3702 | O | WAT | W | 4 | −27.777 | −6.869 | −31.502 | 1.00 | 32.30 | 8 | |
| ATOM | 3703 | O | WAT | W | 5 | 16.593 | 0.034 | 6.219 | 1.00 | 30.62 | 8 | |
| ATOM | 3704 | O | WAT | W | 6 | 14.513 | 2.346 | 3.344 | 1.00 | 31.12 | 8 | |
| ATOM | 3705 | O | WAT | W | 7 | 28.562 | 12.784 | 21.663 | 1.00 | 34.09 | 8 | |
| ATOM | 3706 | O | WAT | W | 8 | 16.086 | 2.555 | 8.816 | 1.00 | 29.73 | 8 | |
| ATOM | 3707 | O | WAT | W | 9 | 31.864 | 20.213 | 8.572 | 1.00 | 28.59 | 8 | |
| ATOM | 3708 | O | WAT | W | 10 | −30.992 | −10.307 | −32.072 | 1.00 | 29.68 | 8 | |
| ATOM | 3709 | O | WAT | W | 11 | −26.050 | 2.758 | −38.362 | 1.00 | 32.55 | 8 | |
| ATOM | 3710 | O | WAT | W | 12 | 27.489 | −8.003 | 7.433 | 1.00 | 33.37 | 8 | |
| ATOM | 3711 | O | WAT | W | 13 | 12.364 | 0.356 | 2.037 | 1.00 | 28.32 | 8 | |
| ATOM | 3712 | O | WAT | W | 14 | 35.876 | 2.813 | 16.912 | 1.00 | 37.61 | 8 | |
| ATOM | 3713 | O | WAT | W | 15 | 35.091 | 0.594 | 15.613 | 1.00 | 33.67 | 8 | |
| ATOM | 3714 | O | WAT | W | 16 | 26.700 | 14.898 | −0.416 | 1.00 | 31.60 | 8 | |
| ATOM | 3715 | O | WAT | W | 17 | 33.877 | −11.857 | −3.921 | 1.00 | 36.78 | 8 | |
| ATOM | 3716 | O | WAT | W | 18 | 33.521 | 9.338 | 18.361 | 1.00 | 36.77 | 8 | |
| ATOM | 3717 | O | WAT | W | 19 | 10.619 | 5.795 | 4.943 | 1.00 | 38.47 | 8 | |
| ATOM | 3718 | O | WAT | W | 20 | 12.148 | 9.034 | 4.695 | 1.00 | 33.68 | 8 | |
| ATOM | 3719 | O | WAT | W | 21 | 21.930 | 2.694 | −8.658 | 1.00 | 29.23 | 8 | |
| ATOM | 3720 | O | WAT | W | 22 | 28.179 | 13.091 | 18.799 | 1.00 | 30.76 | 8 | |
| ATOM | 3721 | O | WAT | W | 23 | 24.493 | 14.521 | 4.080 | 1.00 | 34.88 | 8 | |
| ATOM | 3722 | O | WAT | W | 24 | 19.906 | 6.992 | −13.260 | 1.00 | 32.20 | 8 | |
| ATOM | 3723 | O | WAT | W | 25 | 7.557 | −8.450 | 2.900 | 1.00 | 63.84 | 8 | |
| ATOM | 3724 | O | WAT | W | 26 | −27.367 | 1.228 | −36.920 | 1.00 | 31.57 | 8 | |
| ATOM | 3725 | O | WAT | W | 27 | 29.654 | 11.921 | −1.388 | 1.00 | 29.35 | 8 | |
| ATOM | 3726 | O | WAT | W | 28 | 21.217 | −7.633 | 11.401 | 1.00 | 37.46 | 8 | |
| ATOM | 3727 | O | WAT | W | 29 | 25.864 | 15.796 | 2.006 | 1.00 | 32.25 | 8 | |
| ATOM | 3728 | O | WAT | W | 30 | −24.053 | 1.535 | −40.872 | 1.00 | 33.67 | 8 | |
| ATOM | 3729 | O | WAT | W | 31 | 14.018 | 13.365 | −22.746 | 1.00 | 62.02 | 8 | |
| ATOM | 3730 | O | WAT | W | 32 | 9.697 | 5.905 | 9.452 | 1.00 | 42.01 | 8 | |
| ATOM | 3731 | O | WAT | W | 33 | 18.932 | 3.541 | 17.552 | 1.00 | 58.68 | 8 | |
| ATOM | 3732 | O | WAT | W | 34 | 25.616 | 10.543 | −1.509 | 1.00 | 36.69 | 8 | |
| ATOM | 3733 | O | WAT | W | 35 | −29.998 | −8.101 | −30.511 | 1.00 | 33.22 | 8 | |
| ATOM | 3734 | O | WAT | W | 36 | 35.706 | −8.967 | −12.207 | 1.00 | 33.88 | 8 | |
| ATOM | 3735 | O | WAT | W | 37 | −28.712 | −14.686 | −27.659 | 1.00 | 40.16 | 8 | |
| ATOM | 3736 | O | WAT | W | 38 | 27.173 | −7.960 | −1.547 | 1.00 | 30.64 | 8 | |
| ATOM | 3737 | O | WAT | W | 39 | 36.208 | 10.457 | −1.144 | 1.00 | 58.59 | 8 | |
| ATOM | 3738 | O | WAT | W | 40 | 25.892 | 25.852 | 25.176 | 1.00 | 36.79 | 8 | |
| ATOM | 3739 | O | WAT | W | 41 | −28.690 | −12.596 | −30.204 | 1.00 | 39.60 | 8 | |
| ATOM | 3740 | O | WAT | W | 42 | 11.127 | 0.674 | −6.719 | 1.00 | 36.86 | 8 | |
| ATOM | 3741 | O | WAT | W | 43 | 12.634 | 7.055 | 6.478 | 1.00 | 34.24 | 8 | |
| ATOM | 3742 | O | WAT | W | 44 | 26.064 | −12.501 | −16.222 | 1.00 | 39.99 | 8 | |
| ATOM | 3743 | O | WAT | W | 45 | −23.089 | −17.237 | −33.109 | 1.00 | 42.46 | 8 | |
| ATOM | 3744 | O | WAT | W | 46 | −21.850 | −0.822 | −47.719 | 1.00 | 35.95 | 8 | |
| ATOM | 3745 | O | WAT | W | 47 | 33.872 | −3.162 | −16.877 | 1.00 | 34.13 | 8 | |
| ATOM | 3746 | O | WAT | W | 48 | 24.365 | 34.040 | 18.694 | 1.00 | 41.11 | 8 | |
| ATOM | 3747 | O | WAT | W | 49 | −28.585 | 5.275 | −23.685 | 1.00 | 47.83 | 8 | |
| ATOM | 3748 | O | WAT | W | 50 | 27.720 | 11.812 | −6.004 | 1.00 | 52.01 | 8 | |
| ATOM | 3749 | O | WAT | W | 51 | 31.145 | 11.986 | 22.378 | 1.00 | 36.29 | 8 | |
| ATOM | 3750 | O | WAT | W | 52 | 17.598 | 21.360 | 13.347 | 1.00 | 40.00 | 8 | |
| ATOM | 3751 | O | WAT | W | 53 | −27.143 | −11.537 | −24.167 | 1.00 | 42.92 | 8 | |
| ATOM | 3752 | O | WAT | W | 54 | 21.250 | 3.872 | 18.777 | 1.00 | 62.89 | 8 | |
| ATOM | 3753 | O | WAT | W | 55 | −11.528 | 0.411 | −15.116 | 1.00 | 43.89 | 8 | |
| ATOM | 3754 | O | WAT | W | 56 | −32.837 | −6.837 | −46.162 | 1.00 | 40.49 | 8 | |
| ATOM | 3755 | O | WAT | W | 57 | −13.041 | 3.240 | −39.158 | 1.00 | 44.50 | 8 | |
| ATOM | 3756 | O | WAT | W | 58 | 13.747 | 1.567 | −9.380 | 1.00 | 38.07 | 8 | |
| ATOM | 3757 | O | WAT | W | 59 | 40.365 | 3.671 | 12.390 | 1.00 | 40.05 | 8 | |
| ATOM | 3758 | O | WAT | W | 60 | 34.206 | 3.404 | 18.995 | 1.00 | 35.50 | 8 | |
| ATOM | 3759 | O | WAT | W | 61 | −25.012 | −9.493 | −24.326 | 1.00 | 33.11 | 8 | |
| ATOM | 3760 | O | WAT | W | 62 | 35.341 | 25.261 | 14.453 | 1.00 | 37.72 | 8 | |
| ATOM | 3761 | O | WAT | W | 63 | 25.868 | 5.419 | 20.464 | 1.00 | 42.24 | 8 | |
| ATOM | 3762 | O | WAT | W | 64 | 19.003 | 16.386 | 23.952 | 1.00 | 42.14 | 8 | |
| ATOM | 3763 | O | WAT | W | 65 | −37.060 | −3.848 | −30.377 | 1.00 | 41.53 | 8 | |
| ATOM | 3764 | O | WAT | W | 66 | 20.233 | 10.551 | −11.649 | 1.00 | 38.76 | 8 | |
| ATOM | 3765 | O | WAT | W | 67 | 36.862 | 18.521 | 18.838 | 1.00 | 33.85 | 8 | |
| ATOM | 3766 | O | WAT | W | 68 | −1.871 | 6.647 | −29.708 | 1.00 | 46.30 | 8 | |
| ATOM | 3767 | O | WAT | W | 69 | 11.965 | 3.813 | 4.062 | 1.00 | 40.29 | 8 | |
| ATOM | 3768 | O | WAT | W | 70 | −27.733 | −1.750 | −21.747 | 1.00 | 39.35 | 8 | |
| ATOM | 3769 | O | WAT | W | 71 | 35.651 | −12.751 | −6.391 | 1.00 | 43.81 | 8 | |
| ATOM | 3770 | O | WAT | W | 72 | 20.746 | 16.973 | −11.203 | 1.00 | 69.69 | 8 | |
| ATOM | 3771 | O | WAT | W | 73 | 36.951 | −3.167 | 13.673 | 1.00 | 46.35 | 8 | |
| ATOM | 3772 | O | WAT | W | 74 | 33.452 | 18.001 | 12.333 | 1.00 | 35.28 | 8 | |
| ATOM | 3773 | O | WAT | W | 75 | 39.836 | −4.636 | −17.377 | 1.00 | 41.94 | 8 | |
| ATOM | 3774 | O | WAT | W | 76 | −26.014 | −7.701 | −22.795 | 1.00 | 36.58 | 8 | |
| ATOM | 3775 | O | WAT | W | 77 | 32.112 | 6.847 | 18.892 | 1.00 | 35.48 | 8 | |
| ATOM | 3776 | O | WAT | W | 78 | −24.158 | −14.389 | −45.202 | 1.00 | 49.56 | 8 | |
| ATOM | 3777 | O | WAT | W | 79 | 12.359 | 7.284 | 9.231 | 1.00 | 35.29 | 8 | |
| ATOM | 3778 | O | WAT | W | 80 | −7.718 | −11.828 | −31.638 | 1.00 | 43.35 | 8 | |
| ATOM | 3779 | O | WAT | W | 81 | −4.433 | −8.546 | −27.834 | 1.00 | 46.46 | 8 | |
| ATOM | 3780 | O | WAT | W | 82 | 12.662 | 11.800 | 4.986 | 1.00 | 37.04 | 8 | |
| ATOM | 3781 | O | WAT | W | 83 | 18.628 | 4.051 | −18.939 | 1.00 | 37.36 | 8 | |
| ATOM | 3782 | O | WAT | W | 84 | 41.874 | 12.668 | 12.296 | 1.00 | 64.40 | 8 | |
| ATOM | 3783 | O | WAT | W | 85 | 24.386 | 29.260 | 11.905 | 1.00 | 39.20 | 8 | |
| ATOM | 3784 | O | WAT | W | 86 | −35.916 | −9.236 | −35.846 | 1.00 | 44.00 | 8 | |
| ATOM | 3785 | O | WAT | W | 87 | 24.932 | 26.384 | 21.522 | 1.00 | 45.38 | 8 | |
| ATOM | 3786 | O | WAT | W | 88 | −14.850 | −1.218 | −37.594 | 1.00 | 39.23 | 8 | |
| ATOM | 3787 | O | WAT | W | 89 | −28.949 | −10.251 | −28.386 | 1.00 | 34.73 | 8 | |
| ATOM | 3788 | O | WAT | W | 90 | −15.971 | 11.758 | −33.481 | 1.00 | 66.16 | 8 | |
| ATOM | 3789 | O | WAT | W | 91 | 29.015 | −14.521 | −18.294 | 1.00 | 50.73 | 8 | |
| ATOM | 3790 | O | WAT | W | 92 | −27.883 | −3.366 | −24.498 | 1.00 | 43.48 | 8 | |
| ATOM | 3791 | O | WAT | W | 93 | 19.046 | 23.268 | 24.787 | 1.00 | 48.24 | 8 | |
| ATOM | 3792 | O | WAT | W | 94 | 10.369 | 4.017 | 7.310 | 1.00 | 39.43 | 8 | |
| ATOM | 3793 | O | WAT | W | 95 | 35.601 | 2.131 | −10.164 | 1.00 | 44.36 | 8 | |
| ATOM | 3794 | O | WAT | W | 96 | 17.848 | 24.939 | 17.240 | 1.00 | 43.72 | 8 | |
| ATOM | 3795 | O | WAT | W | 97 | 19.195 | −7.795 | 14.928 | 1.00 | 46.11 | 8 | |
| ATOM | 3796 | O | WAT | W | 98 | 41.553 | 9.708 | 9.864 | 1.00 | 50.38 | 8 | |
| ATOM | 3797 | O | WAT | W | 99 | −20.658 | 12.851 | −28.172 | 1.00 | 48.34 | 8 | |
| ATOM | 3798 | O | WAT | W | 100 | 23.684 | 14.223 | 26.217 | 1.00 | 56.84 | 8 | |
| ATOM | 3799 | O | WAT | W | 101 | −9.687 | −13.780 | −31.210 | 1.00 | 41.65 | 8 | |
| ATOM | 3800 | O | WAT | W | 102 | −12.758 | 6.302 | −35.847 | 1.00 | 44.53 | 8 | |
| ATOM | 3801 | O | WAT | W | 103 | 34.728 | 8.879 | −6.401 | 1.00 | 54.55 | 8 | |
| ATOM | 3802 | O | WAT | W | 104 | 21.203 | −8.624 | 20.819 | 1.00 | 49.17 | 8 | |
| ATOM | 3803 | O | WAT | W | 105 | −31.852 | −0.946 | −28.601 | 1.00 | 40.67 | 8 | |
| ATOM | 3804 | O | WAT | W | 106 | −9.893 | 3.411 | −35.561 | 1.00 | 45.86 | 8 | |
| ATOM | 3805 | O | WAT | W | 107 | 39.989 | 1.169 | 12.158 | 1.00 | 41.22 | 8 | |
| ATOM | 3806 | O | WAT | W | 108 | −29.350 | −1.852 | −27.097 | 1.00 | 41.79 | 8 | |
| ATOM | 3807 | O | WAT | W | 109 | 32.061 | 6.131 | −8.513 | 1.00 | 40.19 | 8 | |
| ATOM | 3808 | O | WAT | W | 110 | −13.807 | 11.289 | −29.680 | 1.00 | 59.37 | 8 | |
| ATOM | 3809 | O | WAT | W | 111 | 7.858 | −11.260 | 0.049 | 1.00 | 60.48 | 8 | |
| ATOM | 3810 | O | WAT | W | 112 | 16.627 | 1.411 | −12.612 | 1.00 | 40.75 | 8 | |
| ATOM | 3811 | O | WAT | W | 113 | −19.983 | 14.426 | −30.108 | 1.00 | 60.48 | 8 | |
| ATOM | 3812 | O | WAT | W | 114 | 12.245 | 18.538 | −24.003 | 1.00 | 82.55 | 8 | |
| ATOM | 3813 | O | WAT | W | 115 | 39.571 | 15.004 | 13.995 | 1.00 | 37.84 | 8 | |
| ATOM | 3814 | O | WAT | W | 116 | 33.463 | −5.785 | −20.431 | 1.00 | 58.24 | 8 | |
| ATOM | 3815 | O | WAT | W | 117 | −26.072 | 7.203 | −21.426 | 1.00 | 55.49 | 8 | |
| ATOM | 3816 | O | WAT | W | 118 | 18.188 | 20.859 | 6.035 | 1.00 | 42.20 | 8 | |
| ATOM | 3817 | O | WAT | W | 119 | 5.384 | −1.239 | −31.530 | 1.00 | 59.18 | 8 | |
| ATOM | 3818 | O | WAT | W | 120 | 20.262 | −15.072 | −13.492 | 1.00 | 51.73 | 8 | |
| ATOM | 3819 | O | WAT | W | 121 | 30.189 | 11.922 | 24.851 | 1.00 | 49.55 | 8 | |
| ATOM | 3820 | O | WAT | W | 122 | 10.788 | −2.015 | 15.189 | 1.00 | 59.50 | 8 | |
| ATOM | 3821 | O | WAT | W | 123 | −7.050 | −8.261 | −24.625 | 1.00 | 45.64 | 8 | |
| ATOM | 3822 | O | WAT | W | 124 | 18.191 | 23.083 | 19.249 | 1.00 | 41.19 | 8 | |
| ATOM | 3823 | O | WAT | W | 125 | 43.545 | −1.639 | 7.512 | 1.00 | 69.29 | 8 | |
| ATOM | 3824 | O | WAT | W | 126 | −14.472 | 0.948 | −39.333 | 1.00 | 46.33 | 8 | |
| ATOM | 3825 | O | WAT | W | 127 | −31.621 | −6.535 | −29.094 | 1.00 | 39.11 | 8 | |
| ATOM | 3826 | O | WAT | W | 128 | −35.231 | −1.122 | −27.823 | 1.00 | 49.91 | 8 | |
| ATOM | 3827 | O | WAT | W | 129 | −19.094 | 13.991 | −27.043 | 1.00 | 56.45 | 8 | |
| ATOM | 3828 | O | WAT | W | 130 | 38.995 | −6.826 | −15.834 | 1.00 | 50.99 | 8 | |
| ATOM | 3829 | O | WAT | W | 131 | −11.364 | 6.658 | −38.443 | 1.00 | 49.38 | 8 | |
| ATOM | 3830 | O | WAT | W | 132 | −10.858 | −6.847 | −15.161 | 1.00 | 45.27 | 8 | |
| ATOM | 3831 | O | WAT | W | 133 | 33.263 | 13.504 | 27.027 | 1.00 | 49.51 | 8 | |
| ATOM | 3832 | O | WAT | W | 134 | −9.470 | 5.931 | −46.144 | 1.00 | 79.22 | 8 | |
| ATOM | 3833 | O | WAT | W | 135 | 17.824 | 26.088 | 20.282 | 1.00 | 56.19 | 8 | |
| ATOM | 3834 | O | WAT | W | 136 | 14.973 | 20.317 | 5.379 | 1.00 | 60.66 | 8 | |
| ATOM | 3835 | O | WAT | W | 137 | 9.146 | 7.236 | 3.036 | 1.00 | 47.76 | 8 | |
| ATOM | 3836 | O | WAT | W | 138 | 25.903 | 13.234 | −2.501 | 1.00 | 41.93 | 8 | |
| ATOM | 3837 | O | WAT | W | 139 | 29.480 | 14.136 | −11.457 | 1.00 | 62.93 | 8 | |
| ATOM | 3838 | O | WAT | W | 140 | 40.320 | 6.951 | 3.255 | 1.00 | 42.71 | 8 | |
| ATOM | 3839 | O | WAT | W | 141 | −22.104 | 6.671 | −41.503 | 1.00 | 54.26 | 8 | |
| ATOM | 3840 | O | WAT | W | 142 | 14.225 | −11.428 | −3.778 | 1.00 | 70.87 | 8 | |
| ATOM | 3841 | O | WAT | W | 143 | 20.799 | −7.861 | 24.532 | 1.00 | 63.06 | 8 | |
| ATOM | 3842 | O | WAT | W | 144 | 36.018 | −1.336 | −5.770 | 1.00 | 73.31 | 8 | |
| ATOM | 3843 | O | WAT | W | 145 | 17.809 | −8.084 | 11.994 | 1.00 | 63.01 | 8 | |
| ATOM | 3844 | O | WAT | W | 146 | 31.942 | −6.401 | −22.214 | 1.00 | 62.79 | 8 | |
| ATOM | 3845 | O | WAT | W | 147 | −25.476 | −5.436 | −21.368 | 1.00 | 43.05 | 8 | |
| ATOM | 3846 | O | WAT | W | 148 | 22.760 | 4.718 | −22.884 | 1.00 | 54.95 | 8 | |
| ATOM | 3847 | O | WAT | W | 149 | 13.421 | −9.857 | 6.966 | 1.00 | 49.49 | 8 | |
| ATOM | 3848 | O | WAT | W | 150 | 13.765 | 9.827 | −16.866 | 1.00 | 74.60 | 8 | |
| ATOM | 3849 | O | WAT | W | 151 | −32.735 | −9.192 | −28.257 | 1.00 | 51.17 | 8 | |
| ATOM | 3850 | O | WAT | W | 152 | 25.500 | 3.281 | −22.705 | 1.00 | 53.30 | 8 | |
| ATOM | 3851 | O | WAT | W | 153 | 18.235 | −0.257 | 15.603 | 1.00 | 38.90 | 8 | |
| ATOM | 3852 | O | WAT | W | 154 | −6.061 | −14.604 | −28.731 | 1.00 | 54.75 | 8 | |
| ATOM | 3853 | O | WAT | W | 155 | 40.951 | 8.546 | 0.629 | 1.00 | 62.07 | 8 | |
| ATOM | 3854 | O | WAT | W | 156 | 32.698 | 22.571 | 7.376 | 1.00 | 53.61 | 8 | |
| ATOM | 3855 | O | WAT | W | 157 | −30.708 | 0.047 | −42.456 | 1.00 | 58.10 | 8 | |
| ATOM | 3856 | O | WAT | W | 158 | −19.452 | −17.048 | −28.371 | 1.00 | 49.53 | 8 | |
| ATOM | 3857 | O | WAT | W | 159 | −34.314 | −0.554 | −35.382 | 1.00 | 48.91 | 8 | |
| ATOM | 3858 | O | WAT | W | 160 | 6.903 | 3.894 | −31.587 | 1.00 | 70.11 | 8 | |
| ATOM | 3859 | O | WAT | W | 161 | −30.049 | 2.673 | −39.111 | 1.00 | 52.69 | 8 | |
| ATOM | 3860 | O | WAT | W | 162 | 8.467 | 12.865 | −17.494 | 1.00 | 65.54 | 8 | |
| ATOM | 3861 | O | WAT | W | 163 | 33.864 | 18.806 | 10.016 | 1.00 | 44.82 | 8 | |
| ATOM | 3862 | O | WAT | W | 164 | 10.938 | −9.972 | −8.363 | 1.00 | 54.44 | 8 | |
| ATOM | 3863 | O | WAT | W | 165 | −20.769 | 15.416 | −25.966 | 1.00 | 61.44 | 8 | |
| ATOM | 3864 | O | WAT | W | 166 | 29.110 | −7.868 | 4.834 | 1.00 | 59.06 | 8 | |
| ATOM | 3865 | O | WAT | W | 167 | 16.795 | 17.929 | −7.581 | 1.00 | 62.10 | 8 | |
| ATOM | 3866 | O | WAT | W | 168 | −4.711 | −12.245 | −31.586 | 1.00 | 50.04 | 8 | |
| ATOM | 3867 | O | WAT | W | 169 | 19.618 | 32.961 | 13.177 | 1.00 | 49.55 | 8 | |
| ATOM | 3868 | O | WAT | W | 170 | 28.062 | 25.357 | 22.310 | 1.00 | 57.60 | 8 | |
| ATOM | 3869 | O | WAT | W | 171 | 0.867 | −2.085 | 3.050 | 1.00 | 65.30 | 8 | |
| ATOM | 3870 | O | WAT | W | 172 | −21.887 | 16.627 | −31.029 | 1.00 | 70.31 | 8 | |
| ATOM | 3871 | O | WAT | W | 173 | 25.330 | −14.416 | −19.149 | 1.00 | 44.85 | 8 | |
| ATOM | 3872 | O | WAT | W | 174 | 13.027 | −0.641 | −7.690 | 1.00 | 40.87 | 8 | |
| ATOM | 3873 | O | WAT | W | 175 | 29.491 | 15.933 | −2.541 | 1.00 | 46.06 | 8 | |
| ATOM | 3874 | O | WAT | W | 176 | 28.804 | 2.041 | −24.955 | 1.00 | 50.03 | 8 | |
| ATOM | 3875 | O | WAT | W | 177 | −10.519 | 3.857 | −38.349 | 1.00 | 56.56 | 8 | |
| ATOM | 3876 | O | WAT | W | 178 | −25.503 | −15.400 | −42.858 | 1.00 | 45.25 | 8 | |
| ATOM | 3877 | O | WAT | W | 179 | −37.796 | −1.357 | −30.241 | 1.00 | 63.19 | 8 | |
| ATOM | 3878 | O | WAT | W | 180 | −3.515 | −12.703 | −37.655 | 1.00 | 72.93 | 8 | |
| ATOM | 3879 | O | WAT | W | 181 | 21.438 | −3.901 | 24.689 | 1.00 | 64.51 | 8 | |
| ATOM | 3880 | O | WAT | W | 182 | −16.008 | −13.906 | −23.419 | 1.00 | 63.43 | 8 | |
| ATOM | 3881 | O | WAT | W | 183 | 35.740 | −8.463 | −1.794 | 1.00 | 61.46 | 8 | |
| ATOM | 3883 | O | WAT | W | 184 | −10.500 | 2.764 | −15.695 | 1.00 | 70.02 | 8 | |
| ATOM | 3884 | O | WAT | W | 185 | −9.860 | −10.367 | −26.412 | 1.00 | 47.62 | 8 | |
| ATOM | 3885 | O | WAT | W | 186 | −38.554 | −6.542 | −30.237 | 1.00 | 62.93 | 8 | |
| ATOM | 3886 | O | WAT | W | 187 | −32.714 | −3.395 | −28.295 | 1.00 | 48.28 | 8 | |
| ATOM | 3887 | O | WAT | W | 188 | 31.369 | 12.845 | 7.044 | 1.00 | 56.30 | 8 | |
| ATOM | 3888 | O | WAT | W | 189 | −13.854 | −17.255 | −39.219 | 1.00 | 77.03 | 8 | |
| ATOM | 3889 | O | WAT | W | 190 | 38.132 | 5.328 | 1.621 | 1.00 | 53.14 | 8 | |
| ATOM | 3890 | O | WAT | W | 191 | −29.743 | −10.731 | −22.814 | 1.00 | 56.88 | 8 | |
| ATOM | 3891 | O | WAT | W | 192 | 16.319 | −7.567 | −19.734 | 1.00 | 68.76 | 8 | |
| ATOM | 3892 | O | WAT | W | 193 | 20.905 | 5.870 | 21.460 | 1.00 | 58.69 | 8 | |
| ATOM | 3893 | O | WAT | W | 194 | −2.078 | −9.343 | −26.709 | 1.00 | 45.32 | 8 | |
| ATOM | 3894 | O | WAT | W | 195 | −27.973 | −17.217 | −34.661 | 1.00 | 50.54 | 8 | |
| ATOM | 3895 | O | WAT | W | 196 | 8.090 | −6.038 | −12.667 | 1.00 | 72.72 | 8 | |
| ATOM | 3896 | O | WAT | W | 197 | 5.456 | −5.313 | −29.937 | 1.00 | 64.35 | 8 | |
| ATOM | 3897 | O | WAT | W | 198 | −17.580 | 12.084 | −27.772 | 1.00 | 61.72 | 8 | |
| ATOM | 3898 | O | WAT | W | 199 | 39.310 | 5.354 | 15.767 | 1.00 | 65.14 | 8 | |
| ATOM | 3899 | O | WAT | W | 200 | 27.072 | −1.823 | −7.016 | 1.00 | 56.78 | 8 | |
| ATOM | 3900 | O | WAT | W | 201 | −33.783 | −9.367 | −42.448 | 1.00 | 59.22 | 8 | |
| ATOM | 3901 | O | WAT | W | 202 | 32.480 | 1.951 | −21.517 | 1.00 | 51.48 | 8 | |
| ATOM | 3902 | O | WAT | W | 203 | 27.509 | 7.904 | −14.495 | 1.00 | 62.04 | 8 | |
| ATOM | 3903 | O | WAT | W | 204 | −6.762 | −19.906 | −37.936 | 1.00 | 67.41 | 8 | |
| ATOM | 3904 | O | WAT | W | 205 | 10.151 | 2.040 | −8.820 | 1.00 | 63.45 | 8 | |
| ATOM | 3905 | O | WAT | W | 206 | 42.308 | 11.982 | 5.704 | 1.00 | 70.95 | 8 | |
| ATOM | 3906 | O | WAT | W | 207 | 32.614 | 9.993 | 21.419 | 1.00 | 65.65 | 8 | |
| ATOM | 3907 | O | WAT | W | 208 | −19.924 | −12.576 | −42.478 | 1.00 | 64.81 | 8 | |
| ATOM | 3908 | O | WAT | W | 209 | −0.031 | 5.343 | −30.924 | 1.00 | 61.37 | 8 | |
| ATOM | 3909 | O | WAT | W | 210 | 7.595 | 4.610 | −1.989 | 1.00 | 63.64 | 8 | |
| ATOM | 3910 | O | WAT | W | 211 | 9.965 | 9.231 | 3.622 | 1.00 | 60.62 | 8 | |
| ATOM | 3911 | O | WAT | W | 212 | 23.641 | 28.537 | 22.061 | 1.00 | 66.92 | 8 | |
| ATOM | 3912 | O | WAT | W | 213 | −4.088 | −10.684 | −29.666 | 1.00 | 66.92 | 8 | |
| ATOM | 3913 | O | WAT | W | 214 | 4.345 | −9.263 | −30.965 | 1.00 | 66.64 | 8 | |
| ATOM | 3914 | O | WAT | W | 215 | 26.160 | 29.184 | 20.917 | 1.00 | 71.88 | 8 | |
| ATOM | 3915 | O | WAT | W | 216 | 37.856 | 8.051 | −5.026 | 1.00 | 69.47 | 8 | |
| ATOM | 3916 | O | WAT | W | 217 | −27.466 | 0.737 | −21.368 | 1.00 | 61.39 | 8 | |
| ATOM | 3918 | O | WAT | W | 218 | 23.412 | −16.483 | −20.202 | 1.00 | 69.69 | 8 | |
| ATOM | 3919 | O | WAT | W | 219 | 28.293 | −13.330 | −8.770 | 1.00 | 67.32 | 8 | |
| ATOM | 3920 | O | WAT | W | 220 | 3.457 | −0.665 | 5.706 | 1.00 | 75.41 | 8 | |
| ATOM | 3921 | O | WAT | W | 221 | 21.431 | −19.910 | −19.507 | 1.00 | 66.40 | 8 | |
| ATOM | 3922 | O | WAT | W | 222 | 35.336 | 2.654 | −5.715 | 1.00 | 72.34 | 8 | |
| ATOM | 3923 | OW0 | WAT | W | 223 | 35.726 | 24.518 | 9.473 | 1.00 | 49.00 | 8 | |
| ATOM | 3924 | OW0 | WAT | W | 224 | 14.105 | 8.614 | −1.895 | 1.00 | 69.00 | 8 | |
| ATOM | 3925 | OW0 | WAT | W | 225 | −5.513 | 12.590 | −37.262 | 1.00 | 69.00 | 8 | |
| ATOM | 3926 | OW0 | WAT | W | 226 | 28.752 | 28.494 | 20.210 | 1.00 | 70.00 | 8 | |
| ATOM | 3927 | OW0 | WAT | W | 227 | 20.227 | 15.904 | −8.842 | 1.00 | 70.00 | 8 | |
| ATOM | 3928 | OW0 | WAT | W | 228 | 7.887 | 3.313 | −4.421 | 1.00 | 71.00 | 8 | |
| ATOM | 3929 | OW0 | WAT | W | 229 | 18.680 | 0.000 | 18.315 | 1.00 | 71.00 | 8 | |
| ATOM | 3930 | OW0 | WAT | W | 230 | −21.527 | −17.229 | −35.367 | 1.00 | 71.00 | 8 | |
| ATOM | 3931 | OW0 | WAT | W | 231 | −32.631 | −10.602 | −30.315 | 1.00 | 72.00 | 8 | |
| ATOM | 3932 | OW0 | WAT | W | 232 | −29.535 | −7.952 | −51.156 | 1.00 | 72.00 | 8 | |
| ATOM | 3933 | OW0 | WAT | W | 233 | 31.358 | 15.241 | 30.315 | 1.00 | 72.00 | 8 | |
| ATOM | 3934 | OW0 | WAT | W | 234 | 14.620 | −11.265 | −22.105 | 1.00 | 72.00 | 8 | |
| ATOM | 3935 | OW0 | WAT | W | 235 | −31.286 | 7.289 | −31.578 | 1.00 | 72.00 | 8 | |
| ATOM | 3936 | OW0 | WAT | W | 236 | 6.255 | 7.289 | 1.895 | 1.00 | 72.00 | 8 | |
| ATOM | 3937 | OW0 | WAT | W | 237 | −3.231 | 23.193 | −21.473 | 1.00 | 73.00 | 8 | |
| ATOM | 3938 | OW0 | WAT | W | 238 | −6.016 | −5.301 | −28.420 | 1.00 | 73.00 | 8 | |
| ATOM | 3939 | OW0 | WAT | W | 239 | 12.255 | −4.639 | −25.262 | 1.00 | 73.00 | 8 | |
| ATOM | 3940 | OW0 | WAT | W | 240 | 8.824 | −0.663 | 12.631 | 1.00 | 73.00 | 8 | |
| ATOM | 3941 | OW0 | WAT | W | 241 | 35.953 | −5.301 | −5.684 | 1.00 | 73.00 | 8 | |
| ATOM | 3942 | OW0 | WAT | W | 242 | −20.326 | 2.651 | −34.104 | 1.00 | 73.00 | 8 | |
| ATOM | 3943 | OW0 | WAT | W | 243 | 17.192 | 23.855 | 22.105 | 1.00 | 73.00 | 8 | |
| ATOM | 3944 | OW0 | WAT | W | 244 | 9.566 | −10.602 | −22.736 | 1.00 | 73.00 | 8 | |
| ATOM | 3945 | OW0 | WAT | W | 245 | −16.351 | −1.325 | −14.526 | 1.00 | 73.00 | 8 | |
| ATOM | 3946 | OW0 | WAT | W | 246 | 26.578 | 26.506 | 2.526 | 1.00 | 74.00 | 8 | |
| ATOM | 3947 | OW0 | WAT | W | 247 | −9.858 | 8.614 | −39.157 | 1.00 | 74.00 | 8 | |

Figures were produced with Ribbons (Carson, J. Appl. Crystallogr. 24:958-961, 1991) or SPOCK.

### Plk1 PBD Binding to Cellular Substrates

HeLa cells were transfected with His/Xpress-tagged Plk1 (residues 326-603 or 326-506) or myc-tagged Plk1 (full-length). They were allowed to recover for 17 hours and then arrested in G2/M by treatment with nocodazole (50 ng/mL) for 14 hours. Cells were lysed in 25 mM Tris/HCl (pH7.5) containing 125 mM NaCl, 0.5% NP-40, 5 mM EDTA, 2 mM DTT, 4 μg/mL pepstatin, 4 μg/mL aprotinin, 4 μg/mL leupeptin, 1 mM Na₃VO₄, 50 mM NaF, and 1 μM microcystin. Lysates were incubated with 5 μL Ni²⁺ beads or 5 μl, α-myc-conjugated beads (Santa Cruz Biotechnology) for 90 minutes at 4° C. Beads were washed four times with lysis buffer. Precipitated proteins were eluted in sample buffer and detected by blotting with polyclonal anti-Cdc25C (Santa Cruz Biotechnology). Point mutations of Plk1 were constructed using the QuickChange site-directed mutagenesis system (Stratagene, La Jolla, Calif.) and verified by DNA sequencing.

### Centrosomal Localization of the Plk1 PBD

U2OS cells were cultured in 8-well chamber slides and arrested in G2/M by treatment with nocodazole (50 ng/mL) for 14 hours. After rinsing with PBS, cells were incubated with 4 μM GST-Plk1 PBD (residues 326-603) and Streptolysin-O (1 U/ml) in permeabilization buffer (25 mM HEPES (pH 7.9), 100 mM KCl, 3 mM NaCl, 200 mM sucrose, 20 mM NaF, 1 mM NaOVO₄) for 20 minutes at 37° C. Cells were fixed in 3% paraformaldehyde/2% sucrose for 10 minutes at room temperature and extracted with a 0.5% Triton X-100 solution containing 20 mM Tris-HCl (pH 7.4), 50 mM NaCl, 300 mM sucrose, and 3 mM MgCl₂ for 10 minutes at Room temperature. Slides were stained with Alexa Fluor 488-conjugated anti-GST (Molecular Probes, Eugene, Oreg.) and monoclonal anti-γ-tubulin (Sigma) antibodies at 4° C. overnight, then stained with a Texas Red conjugated anti-mouse secondary antibody for 60 minutes at room temperature and counterstained with 4 μg/ml DAPI. Cells were examined using a Nikon Eclipse E600 fluorescence microscope equipped with a SPOT RT camera and software (Diagnostic Instruments Livingston, Scotland). Images were analyzed using NIH Image.

### Cell Cycle Analysis

HeLa cells were transfected with wild-type and mutant forms of GFP-tagged Plk1 (residues 326-603) for 32 hours. Media containing floating cells was retained, and attached cells were released from plates by trypsinization. The two cell populations were combined, washed with PBS, and stained with Hoechst 33342 (10 μg/mL) for 30 minutes at 37° C. in DMEM/10% FBS (1×10⁶ cells/mL). Dead cells were stained by incubation with propridium iodide (5 μg/mL) for 5 minutes at 4° C. GFP, Hoechst 33342, and propidium iodide fluorescent signals were quantitated on a FAC Star Plus (Becton Dickinson, Franklin Lakes, N.J.) cell sorting machine using Cell Quest software. Cell cycle analysis of the total live cell population (no propidium iodide staining) and live GFP-expressing cells (no propidium staining and GFP positive) was performed using Modfit 2.0.

### Plk1 Kinase Assays

SF9 cells infected with baculoviral GST-Plk1 (full-length) were lysed in 20 mM Hepes/KOH (pH 7.5), 135 mM NaCl, 1% NP40, 5 mM EGTA, 5 μM 13-mercaptoethanol, 35 mM NaF, 0.5 mM Na₃VO₄, 20 mM β-glycerolphosphate, 3 μM microcystin, 1 μM okadaic acid, 10 μg/mL pepstatin, 10 μg/mL leupeptin, and 10 μg/mL aprotinin. Lysates were incubated for 2 hours at 4° C. with glutathione beads, which were subsequently washed five times with 20 mM Hepes/KOH (pH 7.5), 415 mM NaCl, 0.1% CHAPS, 5 mM EGTA, 5 μM β-mercaptoethanol, 35 mM NaF, and 0.5 mM Na₃VO₄ at 4° C. Bound proteins were eluted with a buffer containing 30 mM glutathione, 50 mM Hepes/KOH (pH 8.0), 25 mM NaCl, 2 mM MgCl₂, 1 mM EGTA, and 5 μM β-mercaptoethanol and dialyzed against 10 mM Hepes, 10 mM NaCl, 1 mM EGTA, 1 mM DTT for 3 hours at 4° C. Kinase reactions were performed in 20 mM Hepes/KOH (pH7.5), 15 mM KCl, 10 mM MgCl₂, 1 mM EGTA, 100 M ATP, 5 μCi γ-[³²P]-ATP, 1 mM DTT, and 0.1 μg/μgL casein for 15 minutes at 30° C. Reaction aliquots were removed at various time points, added to sample buffer, and boiled to arrest phosphorylation. ³²P-incorporation into casein was determined by SDS-PAGE electrophoresis, autoradiography, and densitometry using ImageQuant software (Molecular Dynamics). For peptide activation experiments, 250 μM of the PBD optimal phosphopeptide (MAGPMQSpTPLNGAKK) (SEQ ID NO: 3) or its non-phosphorylated counterpart (MAGPMQSTPLNGAKK) (SEQ ID NO: 34) were pre-incubated with GST-Plk1 for 5 minutes at room temperature.

### Molecular Modeling in Silico

The present invention provides an exemplary crystallized PBD-phosphopeptide complex and the atomic structural coordinates of this complex. The key structural features of the complex, particularly the shape of the substrate binding site, are useful in methods for designing or identifying selective inhibitors of a Polo-like kinase polypeptide, such as Plk-1, and in solving the structures of other proteins with similar features. The structure coordinates of this complex are encoded in a data storage medium, submitted herewith, for use with a computer for graphical three-dimensional representation of the structure and for computer-aided molecular design of new inhibitors. The differences in three-dimensional structure between PLK-1 and related proteins with known structures can be used to optimize selectivity of an inhibitor for PBD. In addition to the structural differences described herein, other differences between Plk-1 and other proteins can also be identified by a skilled artisan.

The three-dimensional atomic structures reported herein can be readily used as a template for selecting potent inhibitors, such as small molecules or peptidomimetics that are designed to “fit” into the binding interface. Methods for designing peptidomimetics using rational drug design are known to the skilled artisan, and are described, for example, in U.S. Pat. Nos. 6,225,076; 6,171,804; and in Han et al. (Bioorg Med. Chem. Lett, 10:39-43, 2000). Peptidomimetics capable of inhibiting complex formation can be identified, for example, through the use of computer modeling using a docking program such as GRAM, DOCK, or AUTODOCK (Dunbrack et al., Folding & Design, 2:27-42, 1997). This procedure can include computer fitting of candidate compounds to a the binding interface of a particular polypeptide to determine whether the shape and chemical structure of the potential ligand will allow it to bind within the structure of the polypeptide. Many methods can be used for this purpose such as, but not limited to, fast shape matching (Dock [Kuntz et al., J. Mol. Biol., 161:269-288, 1982]; Eudock [Perola et al., J. Med. Chem., 43:401-408, 2000]), incremental construction (FlexX [Rarey et al., J Mol Biol, 261, 470-89, 1996]; HAMMERHEAD [Welch et al., Chem. Biol., 3, 449-462, 1996]), TABU search (Pro_Leads [Baxter et al., Proteins 33:367-382, 1998]; SFDock [Hou et al., Protein Eng. 12:639-647, 1999]), genetic algorithms (GOLD [Gold et al., J. Mol. Biol. 267:727-748, 1997]; AutoDock 3.0 [Morris et al., J. Comput. Chem., 19:1639-1662, 1998]; Gambler [Charifson et al., J. Med. Chem., 42:5100-5109, 1999]), evolutionary programming [Gehlhaar et al., Chem. Biol., 2:317-324, 1995], simulated annealing (AutoDock 2.4 [Goodsell et al., Proteins, 8:195-202, 1990]), Monte Carlo simulations (MCDock [Liu et al., J. Comput.-Aided Mol. Des., 13:435-451, 1999]; QXP [McMartin et al., J. Comput.-Aided Mol. Des., 11:333-344, 1997]), and distance geometry (Dockit [Metaphorics LLC, Piemont, Calif. 94611 www.metaphorics.com]).

Those skilled in the art can readily identify many small molecules or fragments as hits. If desired, one can link the different functional groups or small molecules identified by the above procedure into a single, larger molecule. The resulting molecule is likely to be more potent and have higher specificity. The affinity and/or specificity of a hit can also be improved by adding more atoms or fragments that will interact with the target protein. The originally defined target site can be readily expanded to allow further necessary extension. Selected compounds may be systematically modified by computer modeling programs to identify peptidomimetics having the greatest therapeutic potential. Alternatively, candidate compounds are selected from chemical libraries, or are synthesized de novo.

The structural analysis disclosed herein in conjunction with computer modeling allows the selection of a finite number of rational chemical modifications. Thus, using the complex structure disclosed herein and computer modeling, a large number of candidate compounds can be rapidly screened in silico, and the most promising candidates can be identified. Candidate compounds, such as peptidomimetics, are then verified in vitro or in vivo, for example, by determining the effect of the candidate compound on PBD/phosphopeptide binding, Polo-like kinase biological activity, cell cycle regulation, apoptosis, or cell proliferation.

pSer/pThr-Binding Domains Function in The Cellular Response to Genotoxic Stress

Signal transduction by protein kinases in eukaryotes results in the directed assembly of multi-protein complexes at specific locations within the cell (Pawson et al., Science 300:445-52, 2003). This process is particularly evident following DNA damage, where activation of DNA damage kinases results in the formation of protein-protein complexes at discrete foci within the nucleus (Zhou et al., Nature 408:433-9, 2000).

In many cases, kinases directly control the formation of these multi-protein complexes by generating specific phosphorylated-motif sequences; modular binding domains then recognize these short phospho-motifs to mediate protein-protein interactions. The first phosphopeptide-binding modules that were recognized, SH2 and PTB domains, bind specificially to pTyr-containing sequences (Pawson et al., Science 278:2075-80, 1997; Kuriyan et al., Annu Rev Biophys Biomol Struct 26:259-88, 1997; Yaffe, Nat Rev Mol Cell Biol 3:177-86, 2002). As detailed above, a number of modular domains that specifically recognize short pSer/pThr-containing sequences have now been identified, including 14-3-3 proteins, WW domains, FHA domains, and the C-terminal domain of Polo-like kinases (Yaffe et al., Structure 9:R33-8, 2001; Yaffe et al., Curr Opin Cell Biol 13:131-8, 2001; Elia et al., Science 299:1228-31, 2003). All of these pSer/pThr-binding domains participate in cell cycle regulation and the cellular response to genotoxic stress.

### The PTIP Tandem C-Terminal BRCT Pair is Necessary and Sufficient for Phospho-Specific Binding

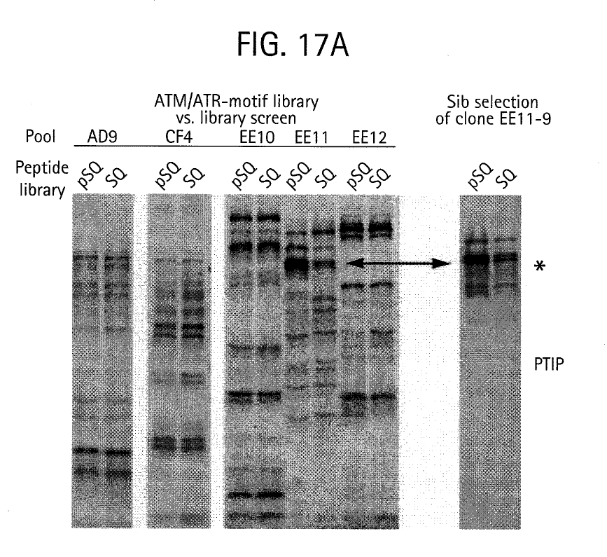

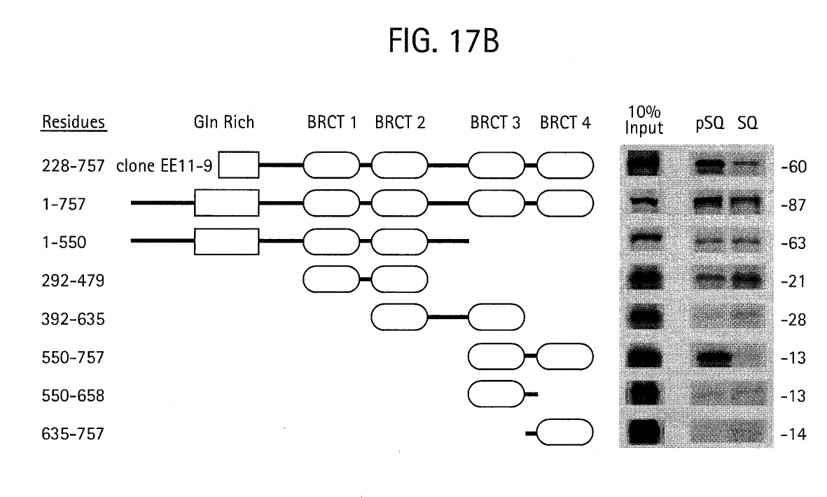

Using the proteomic screening approach (Elia et al., Science 299:1228-31, 2003). described herein, we have now identified novel modular pSer/pThr-binding domains involved in the DNA damage response. Following γ-irradiation, phosphoinositide-like kinases including ATM/ATR and DNA-PK phosphorylate transcription factors, DNA repair proteins, protein kinases and scaffolds on Ser-Gln and Thr-Gln motifs (Abraham, Genes Dev 15:2177-96, 2001). We therefore constructed an oriented peptide library biased to resemble the (pSer or pThr)-Gln motif generated by ATM and ATR (Kim et al., J Biol Chem 274:37538-43, 1999; O'Neill et al., J Biol Chem 275:22719-27, 2000). (FIG. 17A legend). An immobilized form of this library was used in an interaction screen against a library of proteins produced by in vitro expression cloning (Lustig et al., Methods Enzymol 283:83-99, 1997). The amino acids Arg, Lys, and His were intentionally omitted from the degenerate positions in the peptide library to decrease the likelihood of identifying phosphopeptide-binding domains such as 14-3-3, which target basophilic motifs generated by kinases such as AKT, PKA, and PKCs. To control for phosphorylation-independent binding, an identical peptide library was constructed with (Ser or Thr)-Gln substituted for (pSer or pThr)-Gln.

The phosphorylated and non-phosphorylated peptide libraries were immobilized on streptavidin beads, and screened against approximately 96,000 in vitro translated (IVT) polypeptides (960 pools each encoding ˜100 transcripts) over a 10 week period using a high-throughput approach. The majority of IVT products either failed to bind to either of the immobilized peptide libraries or bound slightly better to the non-phosphorylated control (FIG. 17A). Several pools were found to contain cDNAs encoding proteins which bound preferentially to the (pSer or pThr)-Gln library. Pool EEl 1 contained the strongest phosphopeptide-binding clone, EE11-9, which when sib-selected, was found to encode the C-terminal 70% of the human Pax2 trans-activation domain-interacting protein (PTIP) (FIG. 17B) (Lechner et al., Nucleic Acids Res 28:2741-51, 2000; Cho et al., Mol Cell Biol 23:1666-73, 2003). Originally identified in a yeast 2-hybrid screen using Pax2 as bait (Lechner et al., Nucleic Acids Res 28:2741-51, 2000), PTIP appears to play a critical role in the DNA damage response pathway (Cho et al., Mol Cell Biol 23:1666-73, 2003), as well as in facilitating transcriptional responses downstream of TGF-β-Smad2 signaling (Shimizu et al., Mol Cell Biol 21:3901-12, 2001).

Full-length PTIP transcripts also displayed preferential binding to (pSer or pThr)-Gln peptides, though the differential binding was somewhat less pronounced, suggesting that the C-terminal fragment of PTIP likely contains a discrete phosphopeptide binding module. In addition to its Gln-rich region, human PTIP contains 4 BRCT domains, which are known protein-protein interaction modules present in many DNA damage response and cell cycle checkpoint proteins z (Huyton et al., Mutat Res 460:319-32, 2000). A series of deletion constructs was therefore generated and analyzed for phosphopeptide-specific binding (FIG. 17B). A construct containing only the tandem 3^{rd} and 4^{th} BRCT domains showed strong and specific binding to the (pSer or pThr)-Gln library. Constructs of PTIP lacking both of these domains failed to bind or lacked phospho-discrimination. Furthermore, neither the 3^{rd} or 4^{th} BRCT domains alone bound to phosphopeptides, suggesting that the PTIP tandem C-terminal BRCT pair functions as a single module that is necessary and sufficient for phospho-specific binding.

### Tandem BRCT Domains Function as Single Unit to Mediate Phosphopeptide-Binding

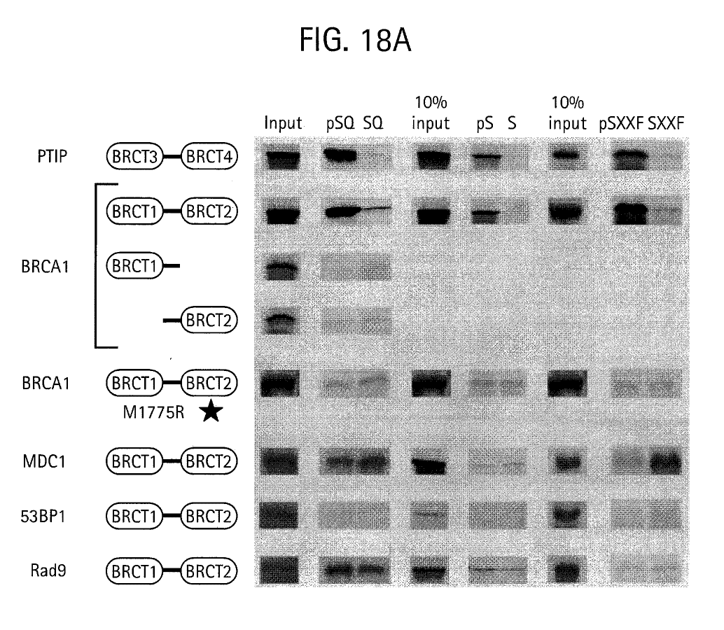

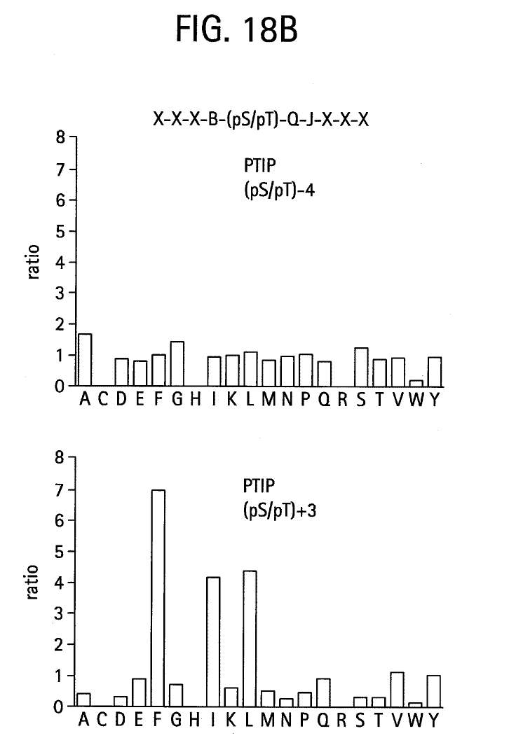

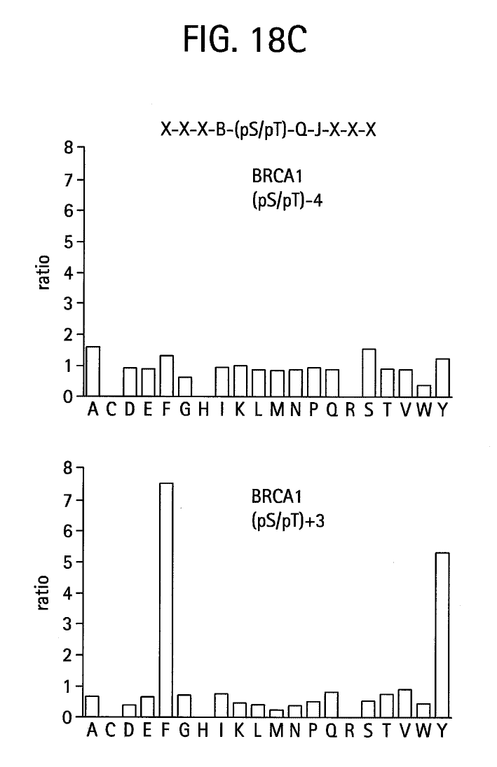

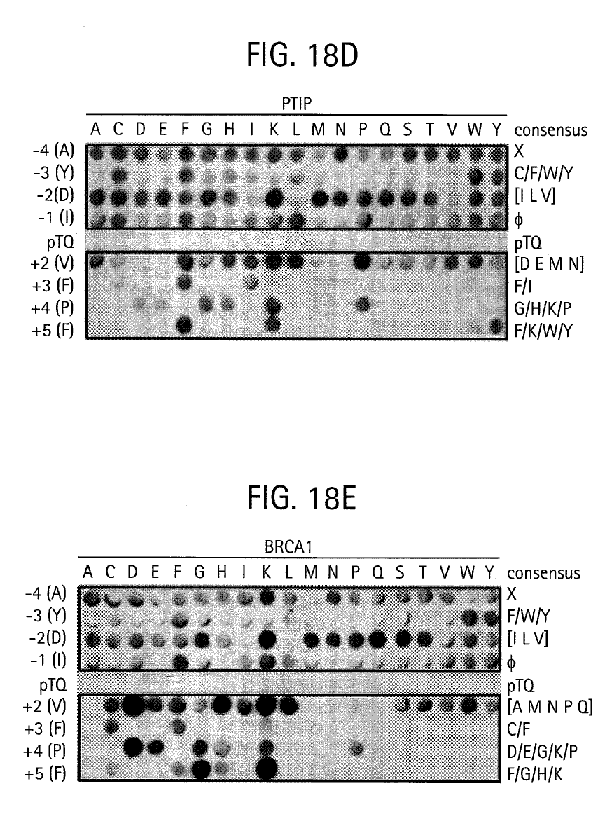

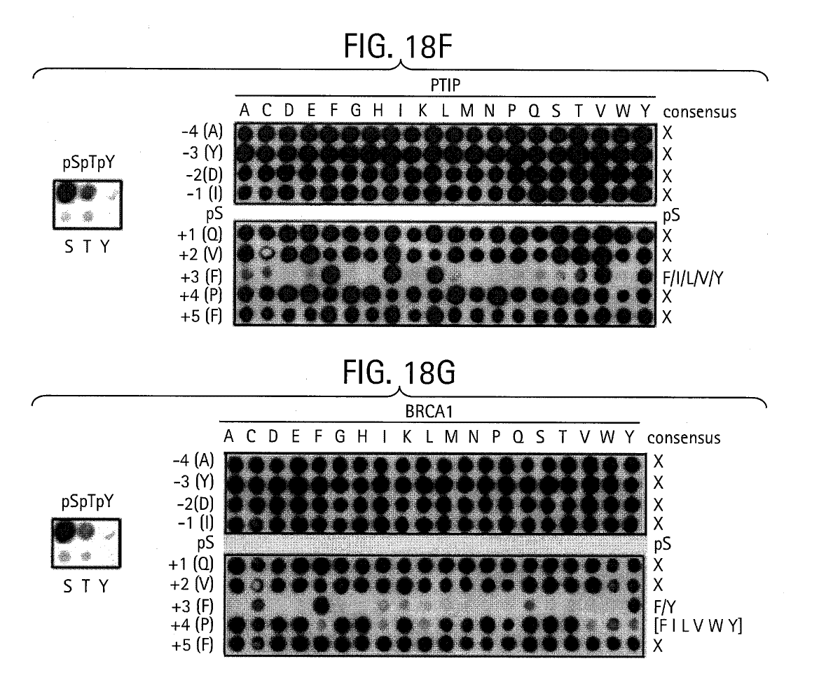

BRCT domains are often found in tandem pairs, or multiple copies of tandem pairs. To investigate whether (pSer- or pThr)-binding is a general feature of these domains, we screened tandem BRCT pairs from a number of other DNA damage proteins (FIG. 18A). Like PTIP, the BRCA1 C-terminal BRCT domains also showed phospho-specific binding. Neither of the BRCA1 BRCT domains alone was sufficient for phospho-specific interactions, again suggesting that the tandem BRCT domains are functioning as a single unit. This observation is in excellent agreement with limited proteolysis and X-ray crystallography studies in which the tandem BRCA 1 BRCT domains together with the inter-domain linker behave as a single stable fragment (Williams et al., Nat Struct Biol 8:838-42, 2001). In contrast to PTIP and BRCA1, phospho-specific binding to the tandem BRCT domains of MDC1 or 53BP1 was not observed, and only a very low amount of phospho-specific binding for Rad9 was detected, suggesting that the phosphopeptide-binding function is present in only a subset of tandem BRCT domains.

### Identification of Optimal Tandem BRCT Domain-Binding Peptide

Modular domains identified by binding to bead-immobilized phosphopeptide libraries are directly amenable to determination of their optimal binding motif by traditional peptide library screening (Yaffe et al., Methods Enzymol 328:157-70, 2000; Elia et al., Science 299:1228-31, 2003). We determined the optimal pSer/pThr binding motifs for the tandem C-terminal BRCTs in PTIP and BRCA1 using (pSer or pThr)-Gln, pSer- and pThr-containing peptide libraries (FIGS. 18B and 18C, Table 46). For the PTIP C-terminal BRCT, the binding motifs using (pSer or pThr)-Gln, pThr-, and two pSer-containing peptide libraries were identified as SEQ ID NO: 110, SEQ ID NO: 111, SEQ ID NO: 112, and SEQ ID NO: 113, respectively (shown in Table 6). For the BRCA1 C-terminal BRCT, the binding motifs using (pSer or pThr)-Gln, pThr-, and two pSer-containing peptide libraries were identified as SEQ ID NO: 114, SEQ ID NO: 115, SEQ ID NO: 116, and SEQ ID NO: 117, respectively (shown in Table 6).

TABLE 6
Phosphoserine and phosphothreonine peptide motif selection
by PTIP and BRCA1 Tandem BRCT motifs
Phosphoserine and Phosphothreonine Peptide
Motif Selection by PTIP and BRCA1 Tandem BRCT Domains

| | | | | | | | | |
|---|---|---|---|---|---|---|---|---|
| −4 | −3 | −2 | −1 | +1 | +2 | +3 | +4 | +5 |

PTIP

| | | | | | | | | | |
|---|---|---|---|---|---|---|---|---|---|
| X | Y (1.5) | G (2.3) | L (2.6) | pS/pT | Q | V (3.8) | F (7.0) | P (1.6) | I (2.9) |
| | | D (1.5) | I (2.5) | | | | | | F (2.7) |
| | | E (1.4) | M (2.5) | | | | | | L (2.4) |
| | | | V (1.9) | | | I (2.8) | L (4.3) | | V (2.0) |
| | | | | | | | I (4.1) | | Y (2.0) |
| X | X | E (1.3) | I (1.4) | PS | F (1.7) | V (1.8) | F | X | I (1.9) |
| | | | M (1.4) | | I (1.5) | T (1.5) | | | F (1.7) |
| | | | V (1.4) | | Q (1.5) | | | | M (1.6) |
| | | | L (1.3) | | Y (1.3) | | | | L (1.4) |
| G (1.6) | Y (1.1) | D (1.2) | L (1.2) | PS | Q (1.3) | V (2.1) | F (2.3) | P (1.2) | Y (1.3) |
| | | E (1.1) | I (1.2) | | I (1.3) | I (1.7) | I (2.3) | | |
| | | | M (1.2) | | P (1.2) | | V (1.8) | | |
| | | | | | | | L (1.7) | | |
| | | | | | | | Y (1.5) | | |
| X | X | X | I (2.1) | PT | Q (1.5) | Y (1.4) | I (1.4) | F (1.5) | A |
| | | | L (1.8) | | F (1.4) | | L (1.3) | Y (1.4) | |
| | | | W (1.3) | | I (1.3) | | V (1.2) | P (1.3) | |

BRCA1

| | | | | | | | | | |
|---|---|---|---|---|---|---|---|---|---|
| X | F (1.7) | D (1.2) | I (1.4) | pS/pT | Q | V (3.1) | F (7.5) | V (1.5) | F (4.5) |
| | Y (1.6) | E (1.1) | V (1.3) | | | T (2.6) | Y (5.2) | P (1.4) | G (1.8) |
| | | | L (1.2) | | | I (2.2) | | | |
| | | | M (1.2) | | | S (1.7) | | | |
| X | R (1.5) | E (1.3) | V (1.4) | pS | F (2.1) | T (1.9) | F | X | F (1.6) |
| | Y (1.4) | D (1.2) | I (1.3) | | Y (1-6) | V (1.7) | | | M (1.4) |
| | | | M (1.3) | | I (1.4) | | | | Y (1.3) |
| | | | | | Q (1.4) | | | | |
| X | X | Y (1.2) | X | pS | Q (1.4) | V (1.2) | F (2.4) | I (1.2) | X |
| | | | | | F (1.3) | I (1.2) | Y (1.5) | | |
| X | E (1.5) | D (1.9) | I (1.6) | pT | Q (1.5) | D (1.5) | F (1.9) | D (1.4) | A |
| | | E (1.5) | L (1.4) | | E (1.4) | Y (1.3) | Y (1.2) | P (1.2) | |
| | | | | | F (1.3) | I (1.2) | | | |

A GST fusion of the PTIP or BRCA1 tandem BRCT domains was screened for binding to four phosphopeptide libraries, which contained the sequences GAXXXB(pS/pT)QJXXXAKKK (SEQ ID NO: 62), GAXXXXpSXXFXXAYKKK (SEQ ID NO: 59), MAXXXXpTXXXXAKKK (SEQ ID NO: 47), and MAXXXXpSpXXXXXAKKK (SEQ ID NO: 58), where X indicates all amino acids except Cys. In the library MAXXXB(pS/pT)QJXXXAKKK (SEQ ID NO: 62) B indicates A, I, L, M, N, P, S, T, V, and J represents a biased mixture of 25% E, 75% X, while X indicates all amino acids except Arg, Cys, His, Lys for all positions in this library. Residues showing strong enrichment are underlined.

Table 6 shows the results of a phosphoserine and phosphothreonine motif selection by PTIP and BRCA1 tandem BRCT domains. A GST fusion of the PTIP or BRCA 1 tandem BRCT domains was screened for binding to three phosphopeptide libraries, which contained the sequences MAXXXB(pS/pT)QJXXXAKKK SEQ ID NO: 57, MAXXXXpTXXXXAKKK SEQ ID NO: 47, and MAXXXXSpXXXXXAKKK SEQ ID NO: 58; where X indicates all amino acids except Cys. In the libraries MAXXXB(pS/pT)QJXXXAKKK (SEQ ID NO: 57) and GAXXXXpSXXFXXAYKKK (SEQ ID NO: 59), B indicates A, I, L, M, N, P, S, T, V; and J represents a biased mixture of 25% E, 75% X, while X indicates all amino acids except Arg, Cys, His, Lys. Residues showing very strong enrichment (ratio>3) are underlined.

PTIP and BRCA1 BRCTs displayed similar, but not identical motifs, with extremely strong selection for aromatic/aliphatic residues, and aromatic residues, respectively, in the (pSer or pThr)+3 position when screened with a (pSer or pThr)-Gln library. Prominent amino acid selection was also observed in the (pSer or pThr)+2 and +5 positions, in addition to more moderate selection at other positions. Because the BRCT domains were isolated in a screen for domains that bind to (pSer or pThr)-Gln motifs, we investigated the relative importance of Gln in the (pSer or pThr)+1 position using individual pThr- or pSer-oriented peptide libraries. This analysis revealed modest selection for Gln in the degenerate+1 position. Furthermore, the absence of a fixed Gln in the +1 position reduced the selection for aromatic and aliphatic residues in the +3 and +5 positions, suggesting that while Gln in the (pSer or pThr)+1 position was not essential, it was clearly a favored residue. In agreement with this finding, we observed considerably stronger binding of the tandem BRCT domains to bead-immobilized (pSer or pThr)-Gln libraries than to libraries containing only a fixed pSer motif (FIG. 18A).

On the basis of peptide library data, we defined an optimal tandem BRCT domain-binding peptide as Y-D-I-(pSer or pThr)-Q-V-F—P—F (SEQ ID NO: 60). Isothermal titration calorimetry (ITC) showed that the optimal phosphoserine-containing peptide bound to the tandem C-terminal BRCTs of PTIP with a dissociation constant of 280 nM, and to the BRCT domains of BRCA1 with a dissociation constant of 400 nM (Table 7). Binding affinity results for GAAYDI-pS-QVFPFAKKK (SEQ ID NO: 123), GAAYDI-pT-QVFPFAKKK (SEQ ID NO: 124), GAAYDI-S-QVFPFAKKK (SEQ ID NO: 125), GAAYDI-T-QVFPFAKKK (SEQ ID NO: 126), GAAYDI-pS-QVFPFAKKK (SEQ ID NO: 127), GAAYDI-S-QVFPFAKKK (SEQ ID NO: 128), and GAAYDI-T-QVFPFAKKK (SEQ ID NO: 129) are shown in Table 7.

TABLE 7
Peptide binding affinities
for the tandem BRCT domains
Table S2. Peptide Binding Affinities
for the Tandem BRCT Domains

| | | (BRCT)₂ | |
|---|---|---|---|
| Peptide | Sequence | Domain | K_{d} |
| BRCTtide-7pS | GAAYDI-pS-QVFPFAKKK | PTIP | 280 nM |
| BRCTtide-7pT | GAAYDI-pT-QVFPFAKKK | PTIP | 14.3 μM |
| BRCTtide-7S | GAAYDI-S-QVFPFAKKK | PTIP | N.D.B. |
| BRCTtide-7T | GAAYDI-T-QVFPFAKKK | PTIP | N.D.B. |
| BRCTtide-7pS | GAAYDI-pS-QVFPFAKKK | BRCA1 | 400 nm |
| BRCTtide-7S | GAAYDI-S-QVFPFAKKK | BRCA1 | N.D.B. |
| BRCTtide-7T | GAAYDI-T-QVFPFAKKK | BRCA1 | N.D.B. |

Isothermal titration calorimetry (ITC) was used to determine binding constants (K_{d}). All observed binding stoichiometries were consistent with a 1:1 complex of protein and phosphopeptide. N.D.B indicates no detectable binding by ITC fog a tandem BRCT domain with a concentration of at least 150 μM. pS and pT denote phosphosarine and phosphopthreonine, respectively.

PTIP and BRCA1 tandem BRCT domains were purified as GST-fusion proteins from E. coli and binding to individual peptides measured by isothermal titration calorimetry. Binding stoichiometries were consistent with a 1:1 complex of protein and phosphopeptide. Replacement of pThr for pSer reduced the affinity of the peptide for the PTIP BRCT domains, while substitution of Thr for pThr abrogated binding altogether.

To further verify motif selection, binding of the tandem BRCT domains to a solid-phase array of immobilized phosphopeptides was performed in which each amino acid flanking the pThr-Gln core (FIGS. 18D and 18E) or flanking the pSer (FIGS. 18F and 18G) in the optimal BRCTtide was varied. The resulting selectivities were generally consistent with the results obtained using oriented peptide libraries in solution. Substitution of pSer for pThr significantly enhanced binding for both PTIP and BRCA1, consistent with the ITC results for PTIP. Substitution of pTyr for pThr eliminated binding altogether, verifying that tandem BRCT domains are pSer/pThr-specific binding modules. As expected, replacement of pThr with Thr, Ser or Tyr abrogated tandem BRCT domain binding.

Tandem BRCT Domain Binding Eliminated by Pre-Incubation with (pSer or pThr)-Gln Peptide Library

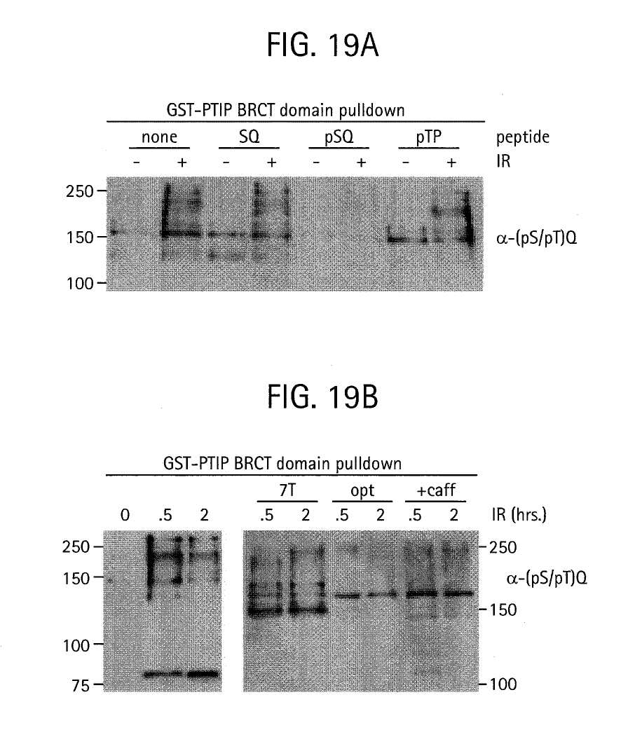

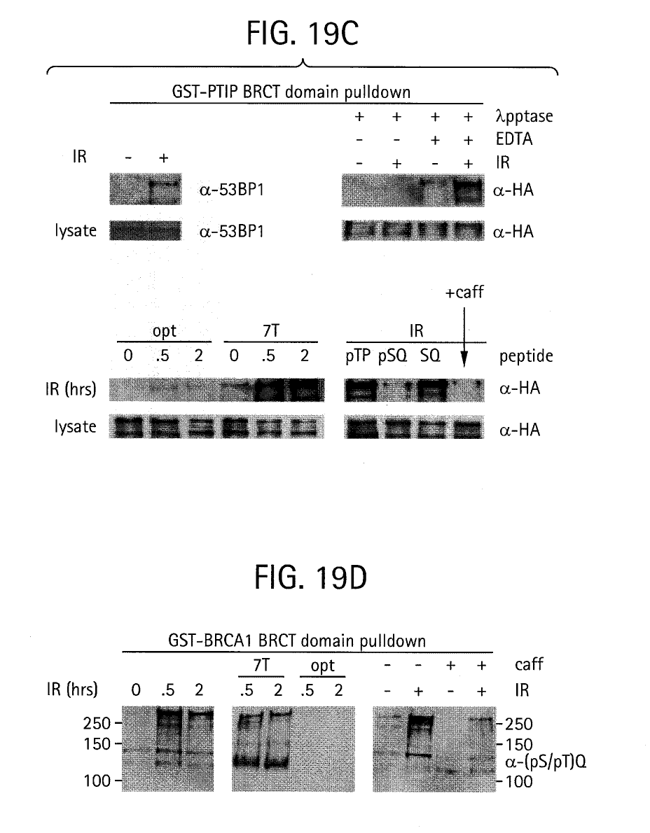

To examine the role of tandem BRCT domains in binding to ATM/ATR/ATX-phosphorylated proteins after DNA damage, U2OS cell lysates, prior to and following 10 Gy of γ-irradiation, were incubated with GST-(BRCT)₂ fusion proteins and blotted with an anti-(pSer or pThr)-Gln motif antibody raised against the phosphorylation motif generated by ATM and ATR (Cell Signaling Technologies) (FIGS. 19A-19D). Following γ-irradiation, both PTIP and BRCA1 tandem C-terminal BRCTs bound to numerous proteins recognized by the anti-ATM/ATR phosphoepitope motif antibody (FIG. 19A). This interaction could be inhibited by pre-incubating the tandem BRCT domains with a (pSer or pThr)-Gln peptide library, but not with a pThr-Pro library or with the non-phosphorylated (Ser or Thr)-Gln library. A time course analysis revealed optimal binding of both the PTIP and BRCA1 BRCT domains to (pSer or pThr)-Gln-containing proteins in irradiated cell lysates at 0.5 and 2 hours after DNA damage (FIGS. 19B and 19D). Binding was largely eliminated by the optimal BRCTtide (opt), but not by its non-phosphorylated analogue (7T), or by pre-treatment of the cells with caffeine to inhibit ATM and ATR prior to γ-irradiation. In both cases where the phospho-specific interaction was eliminated, we observed a ˜170 kDa immunoreactive band in the PTIP BRCT domain pulldowns, but not in the BRCA1 pulldowns; this band likely resulted from an interaction with the PTIP BRCT domains at a site distinct from its phosphopeptide-binding pocket.

### Tandem C-Terminal BRCT Domains are Necessary and Sufficient for Nuclear Foci Formation Following DNA Damage

In response to γ-irradiation, the DNA damage protein 53BP1 undergoes phosphorylation by ATM and facilitates the ability of ATM to phosphorylate additional cellular substrates (Schultz et al., J Cell Biol 151:1381, 2000; Rappold et al., J Cell Biol 153:613-20, 2001; Anderson et al., Mol Cell Biol 21:1719-29, 2001; Abraham, Nat Cell Biol 4:E277-9, 2002; Wang et al., Science 298:1435-8, 2002; Fernandez-Capetillo et al., Nat Cell Biol 4:993-7, 2002; DiTullio, Jr. et al., Nat Cell Biol 4:998-1002, 2002). 53BP1 migrates at a similar Mr as one or more of the bands in FIGS. 19A and 19B and contains multiple potential Ser/Thr-Gln ATM/ATR phosphorylation sites that closely match the optimal PTIP tandem BRCT-binding motif. Endogenous 53BP1 from U2OS cell lysates bound to the tandem C-terminal BRCT domains of PTIP only following DNA damage (FIG. 19C). Similar to the results obtained with the (pSer or pThr)-Gln motif antibody, a time course of cells transfected with HA-tagged 53BP1 revealed optimal binding at 0.5 and 2 hours following γ-irradiation. This binding was inhibited by preincubation with optimal BRCTtide, but was not eliminated by pre-incubation with its non-phosphorylated counterpart. Binding was also eliminated by pre-incubation of the tandem BRCT domains with the (pSer or pThr)-Gln peptide library, but not by pre-incubation with a pThr-Pro library or the non-phosphorylated (Ser or Thr)-Gln library, as well as by treatment with caffeine prior to λ-irradiation or treatment of the lysates with k-phosphatase following irradiation.

Although PTIP was originally identified as a transcriptional control protein, recent data suggests that PTIP might also be involved in DNA damage signaling (Cho et al., Mol Cell Biol 23:1666-73, 2003). Mice homozygous for a PTIP null allele undergo embryonic lethality at E9.5, with evidence of extensive DNA damage and the presence of free DNA ends. Neither fibroblasts nor embryonic stem cells from PTIP null mice could be propagated in culture, and trophoblast cells, which showed decreased viability in general, showed an increased sensitivity to low doses of ionizing radiation (Cho et al., Mol Cell Biol 23:1666-73, 2003). This data, together with our finding that the tandem BRCT domains at the C-terminus of PTIP bind to ATM/ATR phosphorylated proteins, suggested that full-length PTIP might localize at sites of DNA damage in vivo.

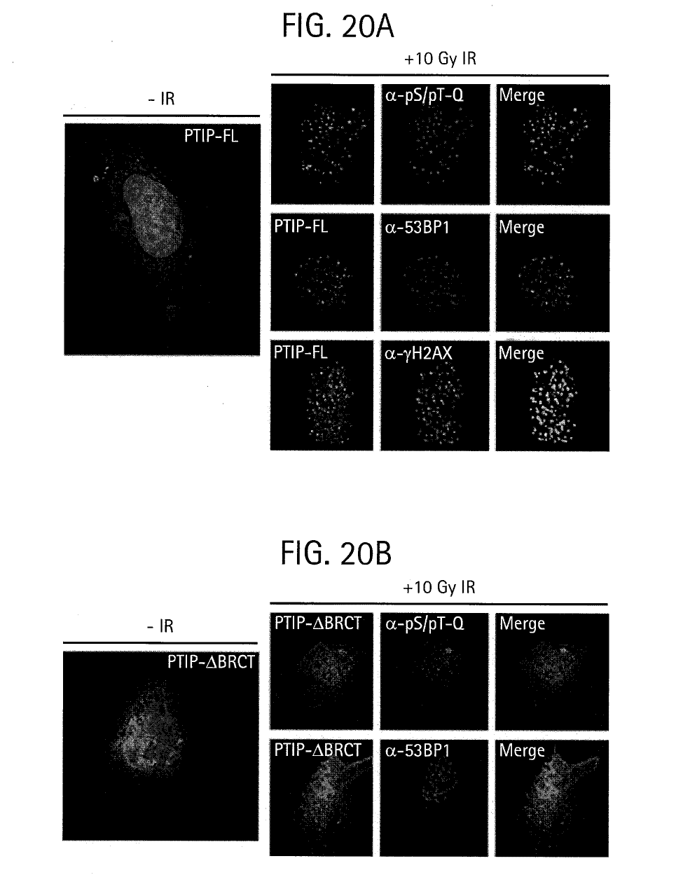

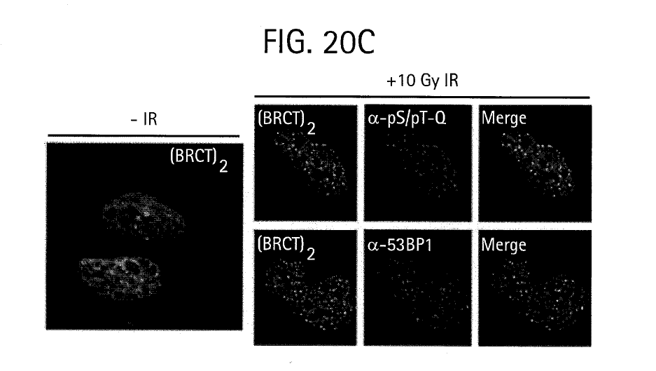

To investigate this, U2OS cells were transfected with GFP fusions of full-length PTIP, PTIP lacking the last two C-terminal BRCT domains, or the isolated tandem C-terminal BRCT domains alone (FIGS. 20A-20C). In the absence of irradiation, PTIP was diffusely nuclear with a small amount of cytosolic staining. Two hours following DNA damage, PTIP re-localized into discrete nuclear foci that significantly co-localized with ATM/ATR phosphoepitopes, 53BP1 and phospho-H2AX (FIG. 20A). Deletion of the C-terminal BRCTs from PTIP resulted in its constitutive diffuse nuclear and cytoplasmic localization and an inability to form foci after DNA damage (FIG. 18B). The isolated PTIP C-terminal tandem BRCT domains, while predominantly diffusely nuclear in the absence of DNA damage, efficiently re-localized into the same punctate nuclear foci after γ-irradiation as full-length PTIP (FIG. 18C). Thus, the tandem C-terminal BRCT domains of PTIP, which are necessary and sufficient for binding to (pSer or pThr)-Gln peptides in solution, are necessary and sufficient for nuclear foci formation by full-length PTIP following DNA damage.

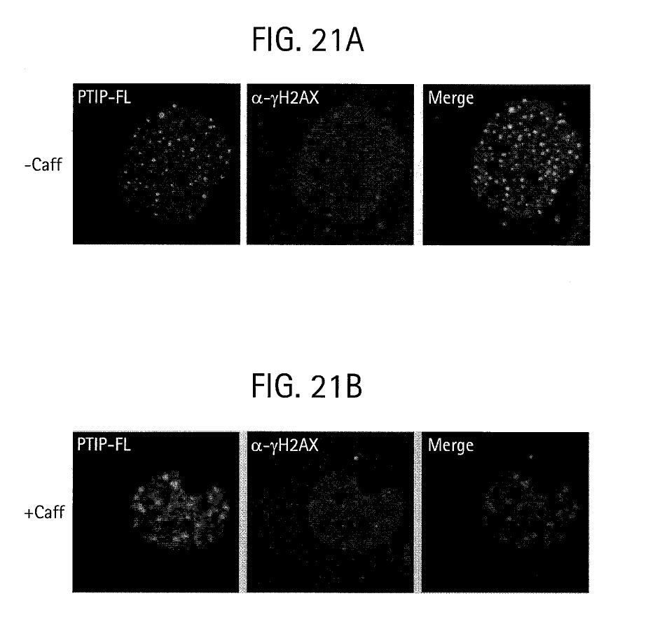

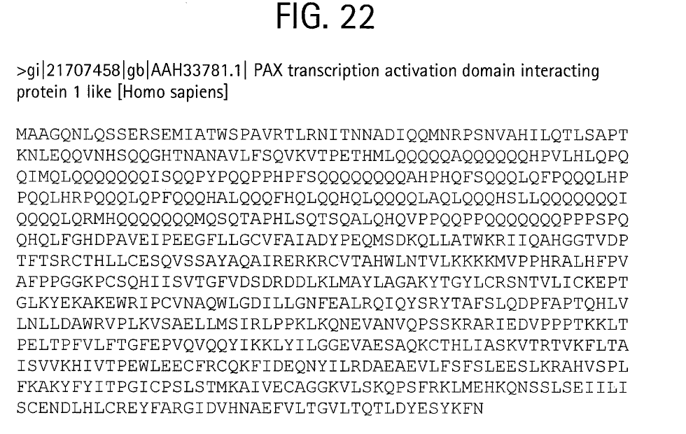

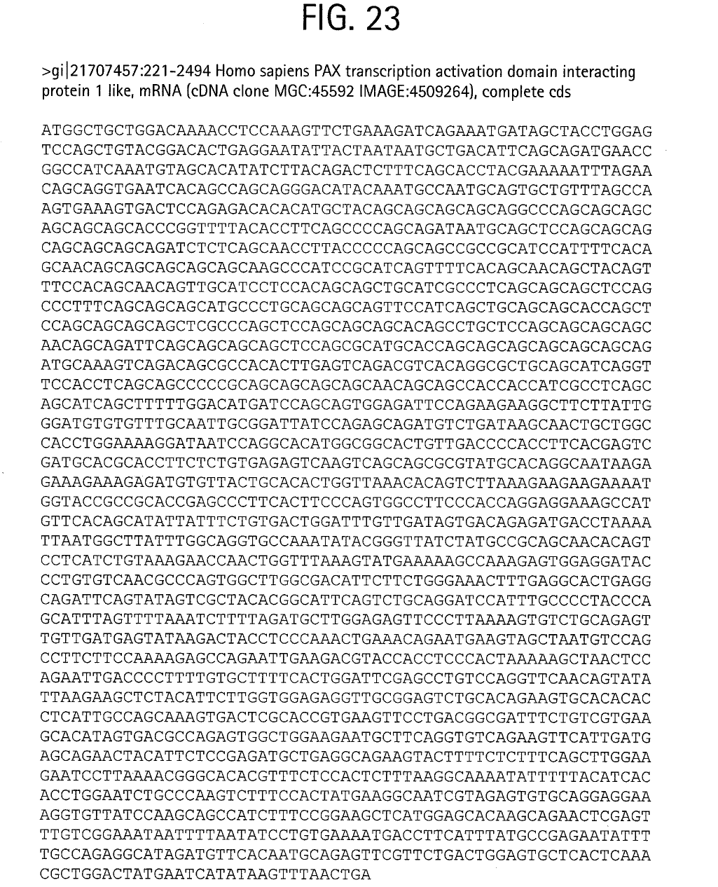

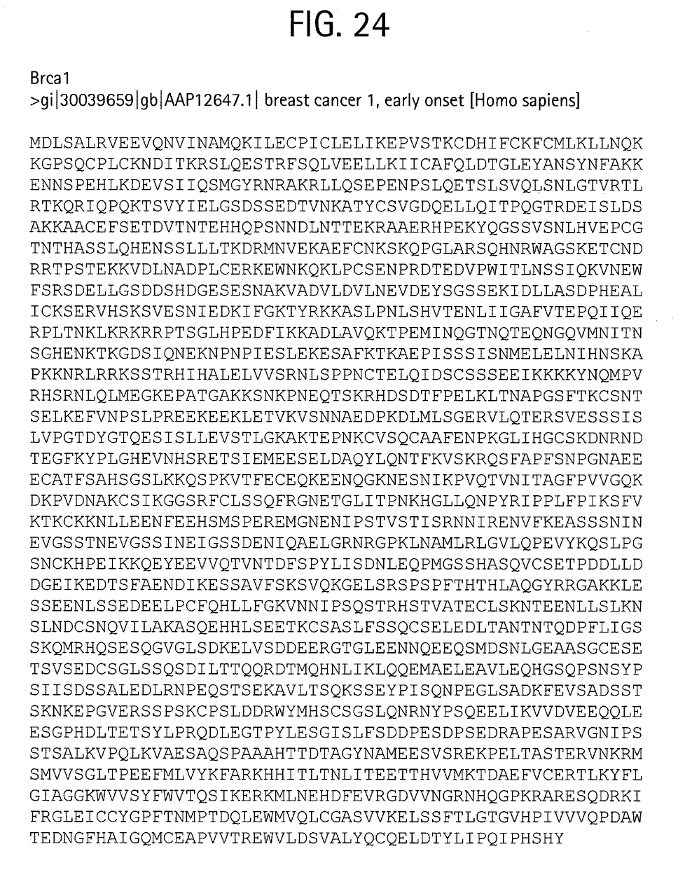

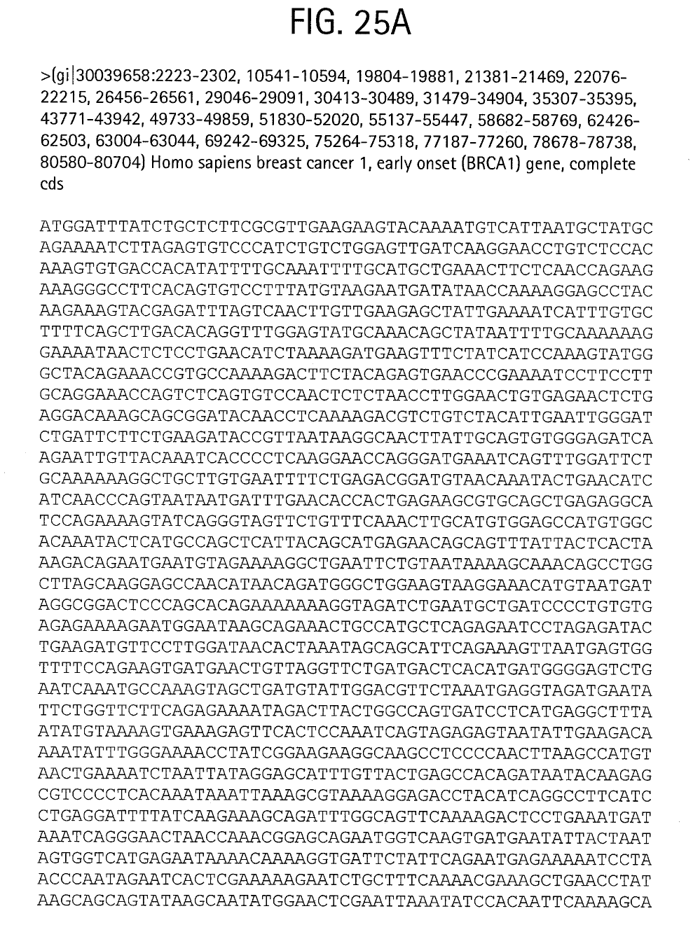

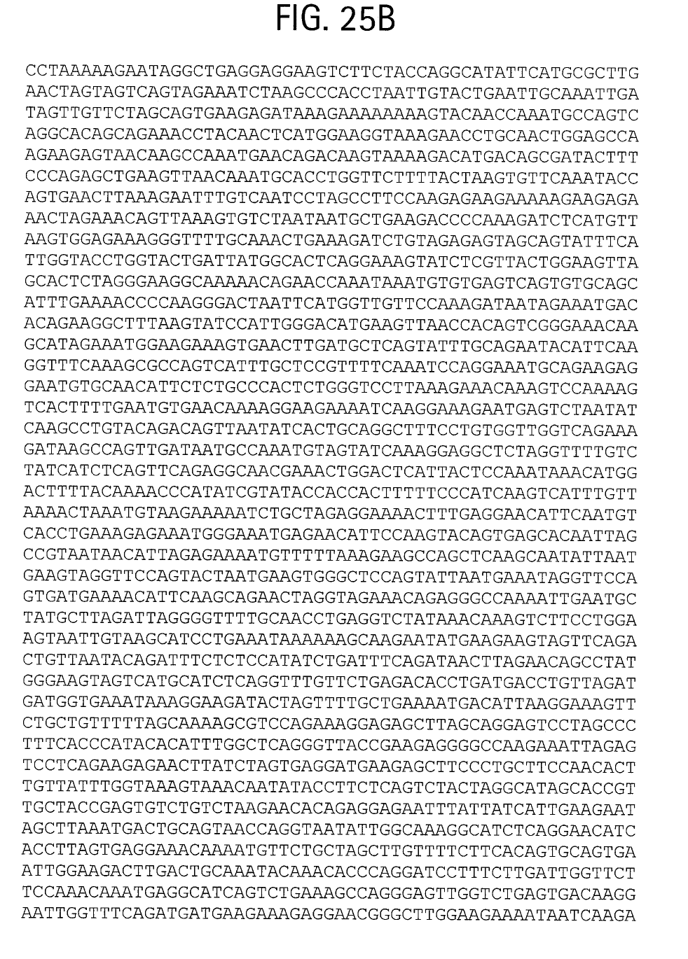

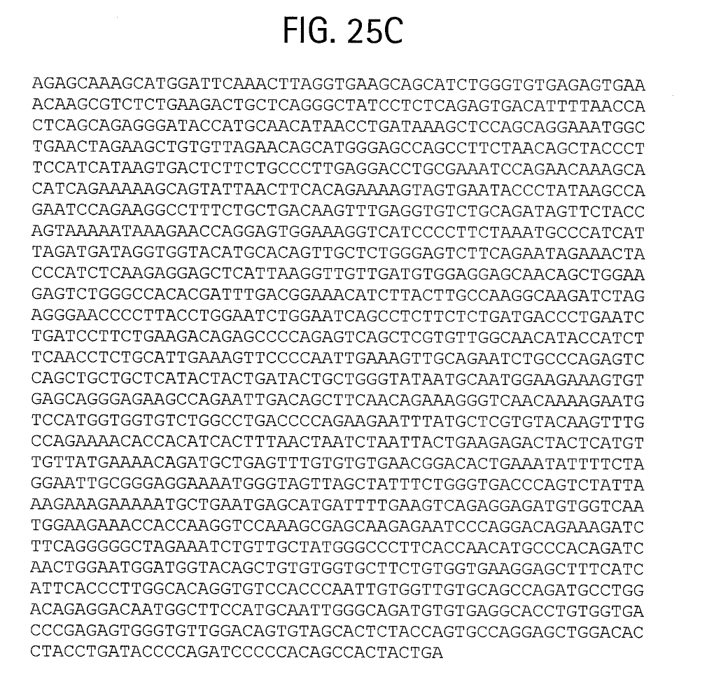

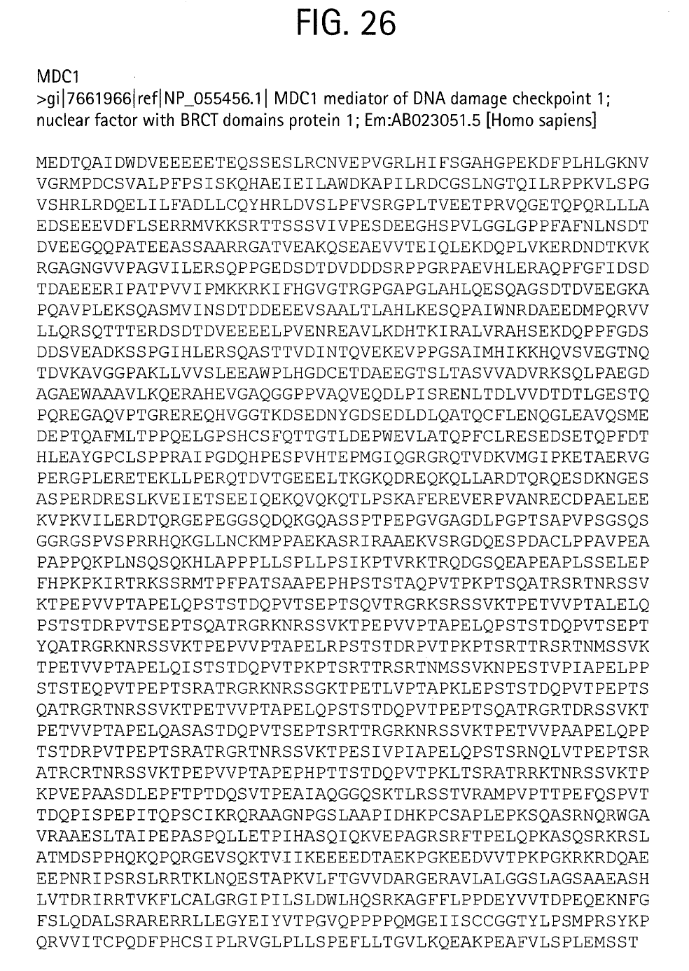

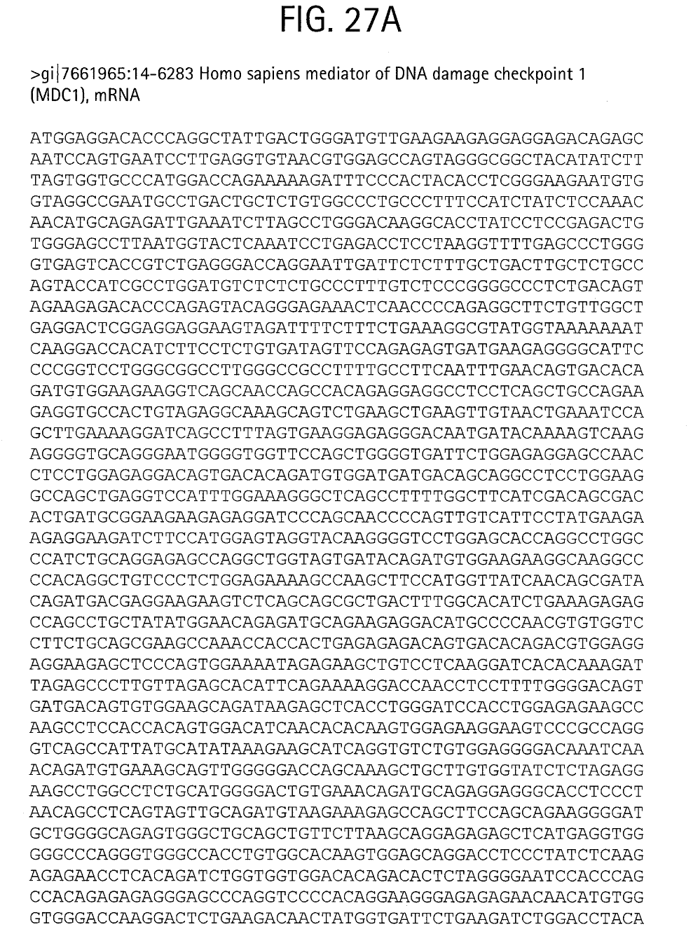

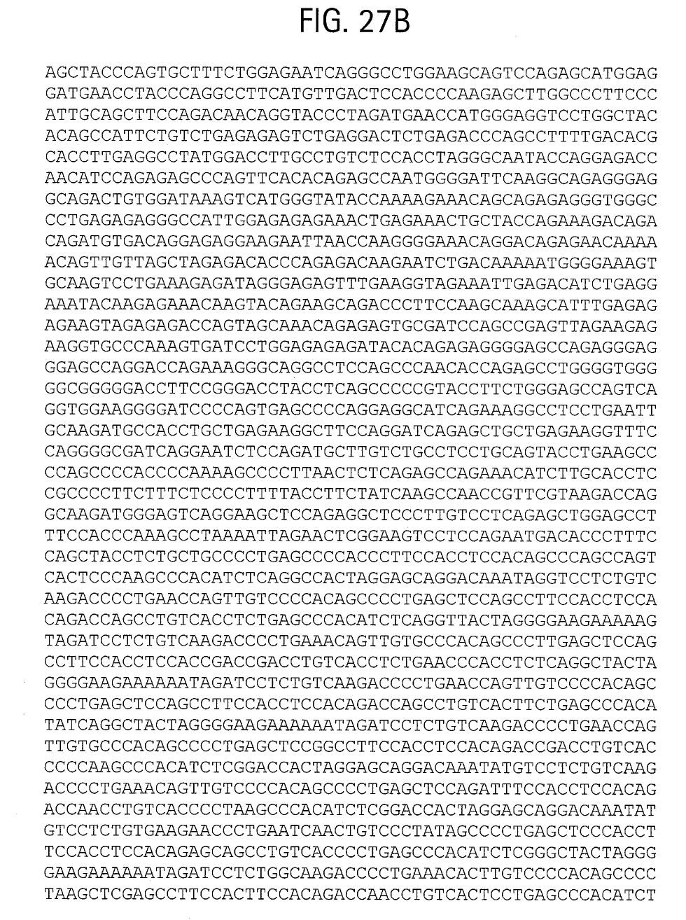

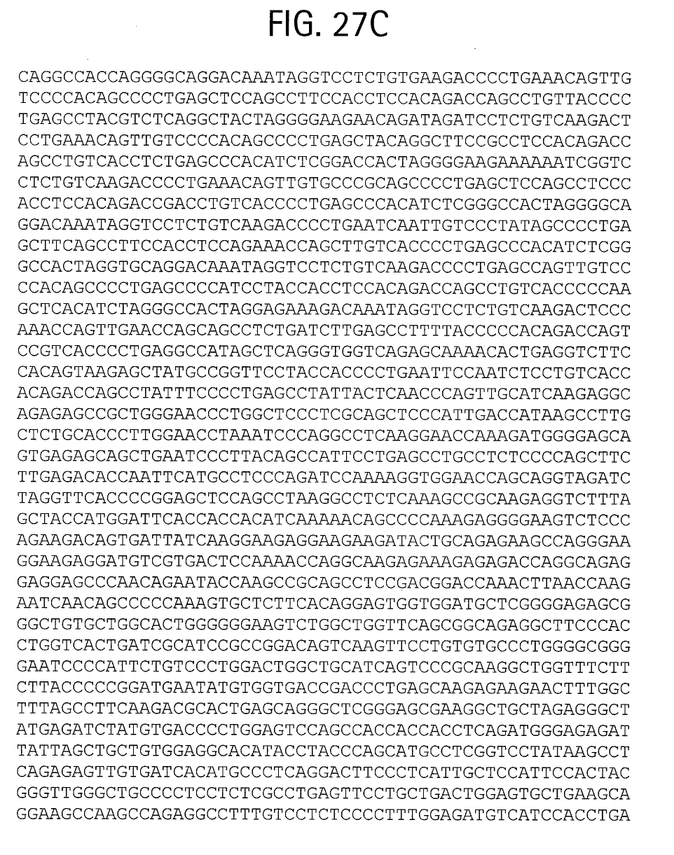

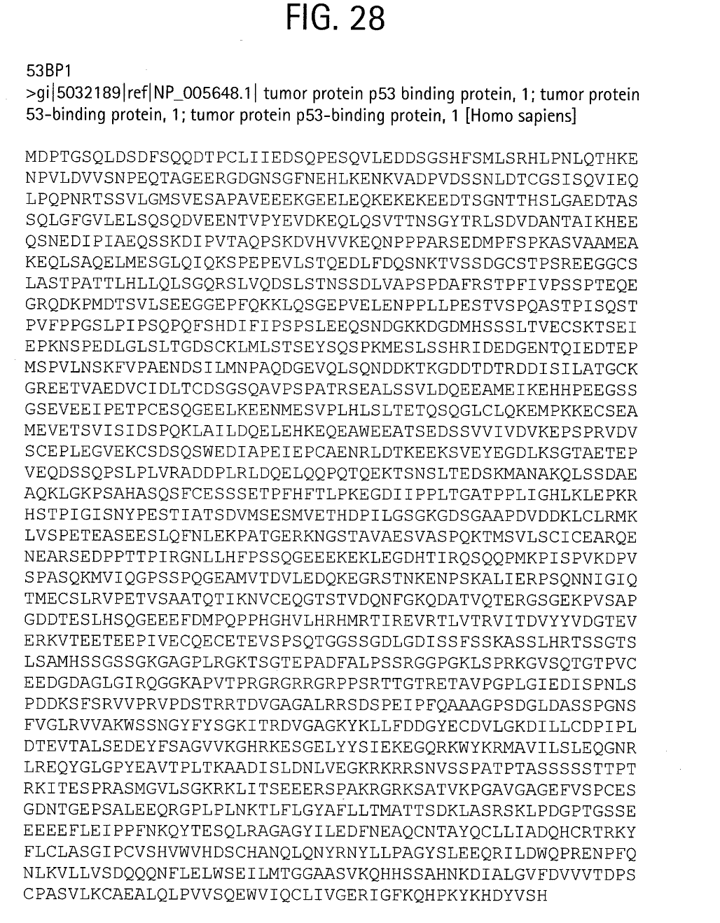

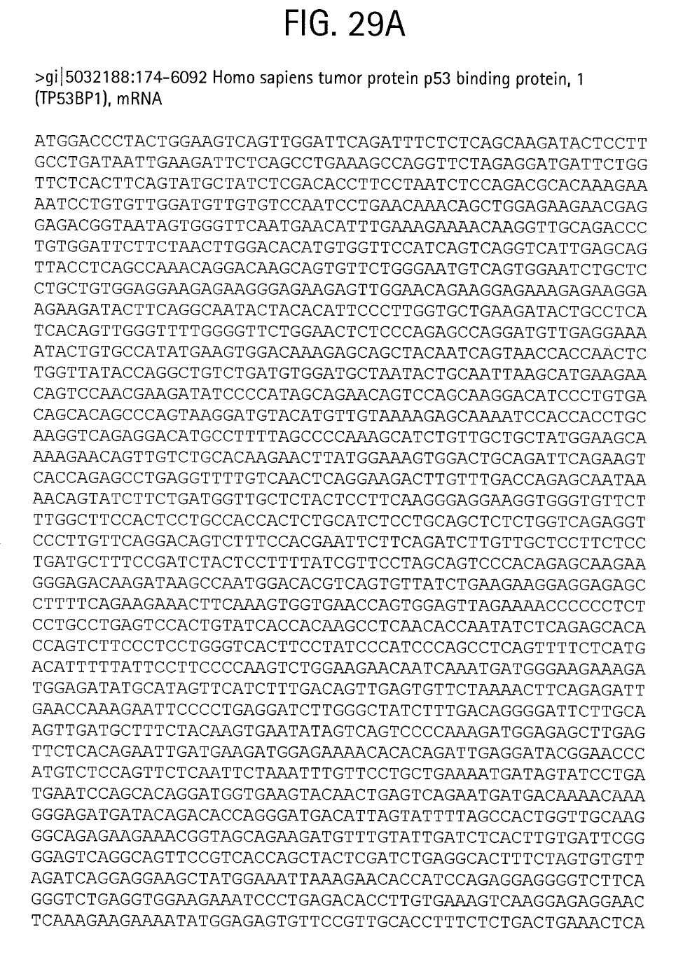

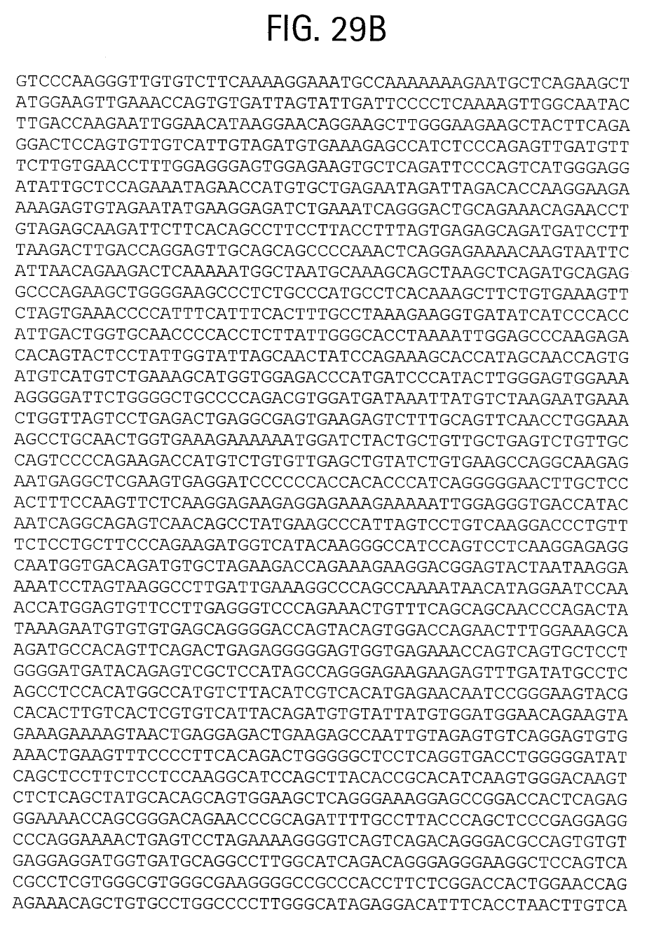

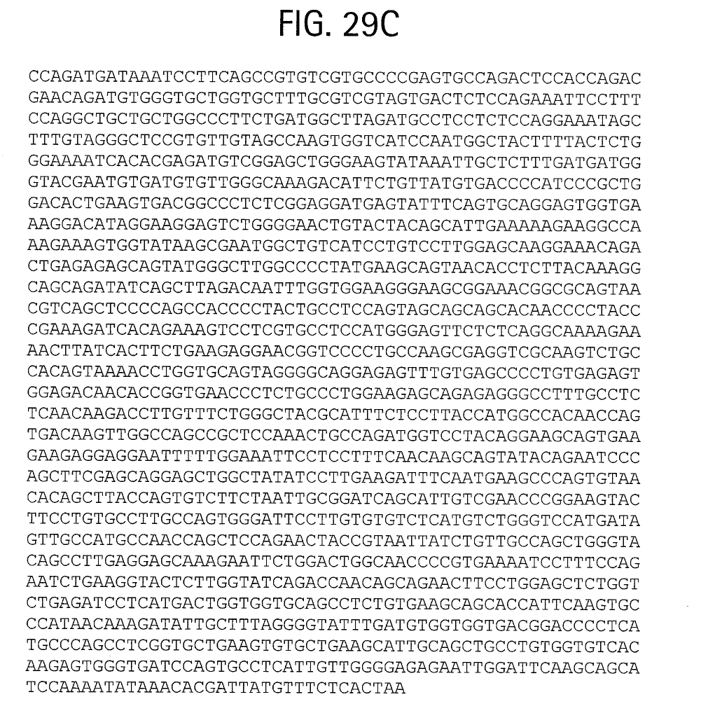

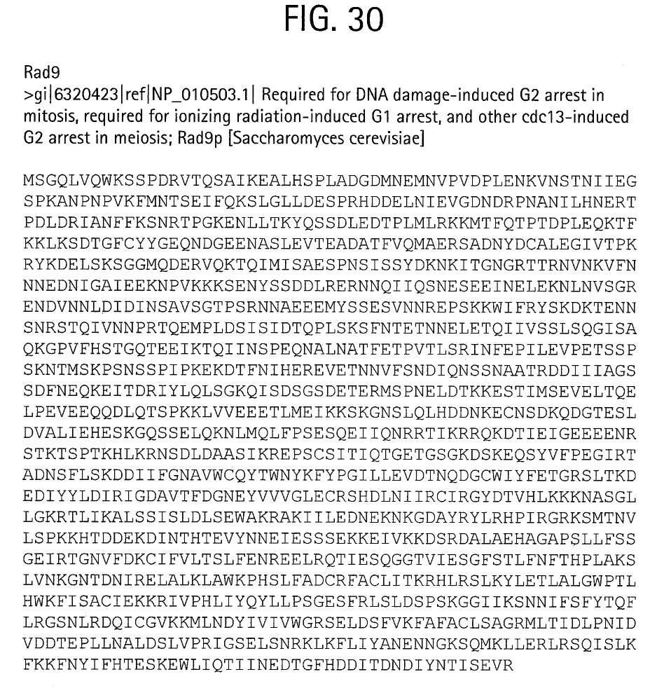

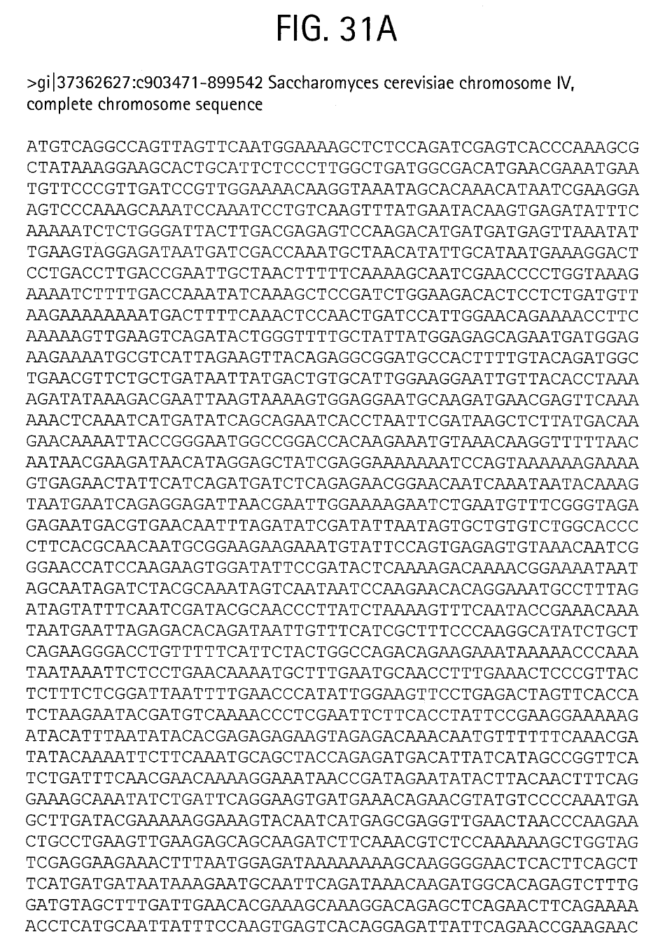

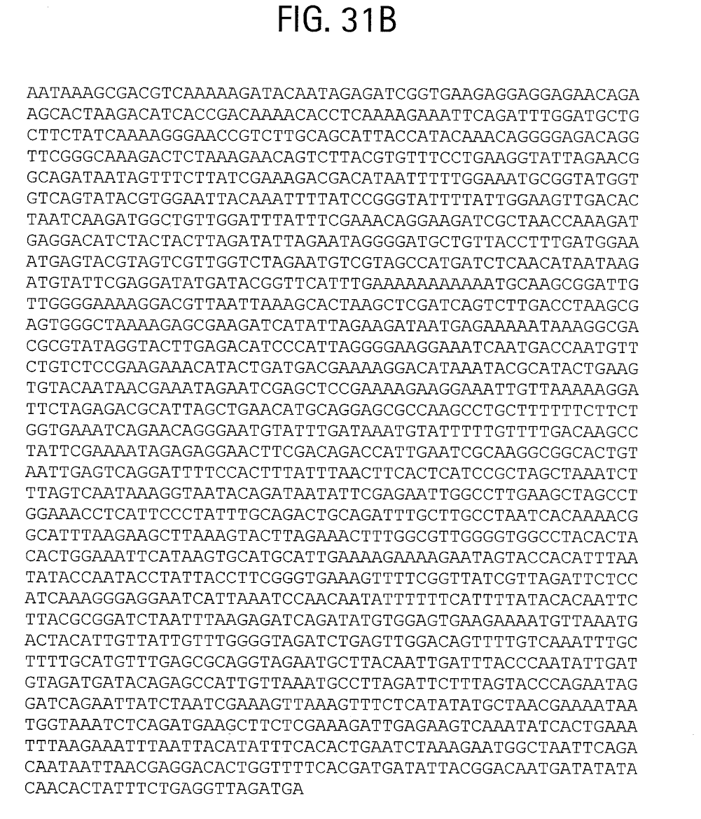

Caffeine attenuates recruitment of PTIP to DNA damage foci in response to ionizing radiation (FIGS. 21A and 21B). U2OS cells transfected with full-length PTIP-GFP cDNA were mock treated or pretreated with 10 mM caffeine for 70 minutes before exposure to 10Gy ionizing radiation. In reponse to IR ionizing radiation, mock-treated U2OS cells formed nuclear foci containing PTIP (in green) and H2AXp (in red); these two proteins co-localize at sites of DNA damage (merge). In response to IR, caffeine treated U2OS cells formed reduced numbers of nuclear foci; PTIP was mislocalized and did not form discrete nuclear foci (in green) and there were reduced numbers of H2AXp (in red) containing foci. These results demonstrate that pretreatment with caffeine effectively abolished co-localization of PT1P and H2AXp (merge).

Our identification of tandem BRCT domains as a new pSer/pThr-binding module targeting ATM and ATR phosphorylation motifs expands the range of functions subserved by this domain in response to DNA damage signaling. Only tandem pairs were observed to function in this capacity, and only a subset of BRCT domains, including those in PTIP and BRCA1, appear to show phospho-specific binding. The important role for tandem BRCT domains as phospho-binding modules is emphasized by the finding that ˜80% of germline mutations in BRCA1 result in C-terminal truncations involving the BRCT region, predisposing women to breast and ovarian cancer (Huyton et al., Mutat Res 460:319-32, 2000). Interestingly, a BRCA1 cancer-associated mutation in the (BRCT)2 module that ablates critical BRCA1 protein interactions, Met17753Arg (M1775R), fails to bind phosphopeptides (FIG. 2A), even though the M1775R crystal structure is nearly identical to that of the wild-type (BRCT)2. The finding that BRCT domains bind to pSer-containing peptides more strongly than to pThr-containing peptides is novel since WW domains, 14-3-3 proteins, FHA domains and Polobox domains either bind pThr-peptides better than pSer peptides, or do not bind to pSer-peptides at all (Verdecia et al., Nat Struct Biol 7:639-43, 2000; Durocher et al., Mol Cell, 6:1169-1182, 2000; Elia et al., Science 299:1228-31, 2003). Intriguingly, ATM and ATR preferentially phosphorylate Ser-Gln over Thr-Gln motifs (Kim et al., J Biol Chem 274:37538-43, 1999), suggesting functional convergence between the motifs generated by phosphoinositide-like kinases and the motifs recognized by BRCT domains. The observed BRCT domain selection for aromatic and aliphatic residues in the (pSer or pThr)+3 and +5 positions within their bound substrates exceeds their modest selection for Gln in the +1 position. Thus, only a subset of ATM/ATR phosphorylated substrates are likely to bind with high affinity. Kinases other than Gln-directed kinases might also generate potential BRCT domain-binding motifs. In addition, the results of our screen provide a molecular rationale for the early embryonic lethality of PTIP knock-out mice with extensive unrepaired DNA ends. The finding that the C-terminal tandem BRCT domains of PTIP bind to ATM/ATR-phosphorylated motifs and localize full-length PTIP to sites of DNA damage strongly suggests that PTIP functions as a key component of the DNA damage response. Interference with the normal process of DNA damage signaling is responsible not only for tumorigenesis but also for tumor cell death in the face of massive DNA damage induced by chemotherapeutic agents, depending on the remaining genetic background of the cancer cell (Scully et al., Nature 408:429-32, 2000). Agents that interfere with DNA damage signaling sensitize tumor cells to killing by radiation and chemotherapy. Thus, the phosphopeptide-binding pocket of tandem BRCT domains constitutes a promising target for anti-cancer drug development.\

### ATM/ATR/ATX Phospho-Motif Screen for Phosphoserine/Threonine Binding Domains

An oriented (pSer/pThr) phosphopeptide library biased toward the phosphorylation motifs for ATM/ATR kinases and its non-phosphorylated counterpart were constructed as follows: biotin-Z-G-Z-G-G-A-X-X-X-B-(pS/pT)-QJ-X-X-X-A-K-K-K SEQ ID NO:35 and biotin-Z-G-Z-G-G-A-X-X-X-B-(S/T)-Q-J-X-X-X-A-K-K-K SEQ ID NO:61, where pS denotes phosphoserine; pT phosphothreonine; Z indicates aminohexanoic acid; B represents a biased mixture of the amino acids A, I, L, M, N, P, S, T, V; and J represents a biased mixture of 25% E and 75% X, where “X” denotes all amino acids except Arg, Cys, His, Lys. Streptavidin beads (Pierce, 75 pmol/μL gel) were incubated with a ten-fold molar excess of each biotinylated library in 50 mM Tris/HCl (pH7.6), 150 mM NaCl, 0.5% NP-40, 1 mM EDTA, 2 mM DTT and washed five times with the same buffer to remove unbound peptide. The bead-immobilized libraries (10 μL of gel) were added to 10 μL of an in vitro translated [³⁵S]-labeled protein pool in 150 μL binding buffer (50 mM Tris/HCl (pH7.6), 150 mM NaCl, 0.5% NP-40, 1 mM EDTA, 2 mM DTT, 8 μg/mL pepstatin, 8 μg/mL aprotinin, 8 μg/mL leupeptin, 800 μM Na₃VO4, 25 mM NaF). Each pool consisted of ˜100 radiolabeled proteins produced by the PROTEOLINK in vitro expression cloning system (Promega, Madison, Wis.). After incubation at 4° C. for 3 hours, the beads were rapidly washed three times 200 μL with binding buffer prior to SDS-PAGE (12.5%) and autoradiography. Positively scoring hits were identified as protein bands that interacted more strongly with the phosphorylated immobilized library than with the unphosphorylated counterpart. Pools containing positively scoring clones were progressively subdivided and re-screened for phosphobinding until single clones were isolated and identified by DNA sequencing.

### Cloning, Expression, and Purification of PTIP and BRCA1

For deletion mapping of the PTIP and BRCA1 BRCT phospho-binding region and for expression of MDC 1, 53BP1 and Rad9 (FIG. 17-18), fragments were generated by PCR for in vitro transcription/translation and cloned into a pcDNA3.1 expression vector (Invitrogen, San Diego, Calif.). For production of recombinant GST-PTIP BRCT domains and GSTBRCA1 BRCT domains, residues 550-757 of PTIP and residues 1634-1863 of BRCA1 were ligated into the EcoRI and NotI sites of pGEX-4T1 (Pharmacia, Peapack, N.J.) and subsequently transformed into DH5a E. Coli. Protein induction occurred at 37° C. for 4 hours or at 25° C. for 16 hours in the presence of 0.4 mM IPTG. For peptide filter blot analysis and measurements of peptide binding affinity by ITC, GSTPTIP BRCT domains (residues 550-757) and GST-BRCA1 BRCT domains (residues 1634-1863) were isolated from bacterial lysates using glutathione agarose, eluted with 40 mM glutathione, and dialyzed into 50 mM Tris/HCl (pH 8.1), 300 mM NaCl. The GFP-PTIP constructs FL (residues 1-757), !BRCT (residues 1-550), or (BRCT)2 (residues 550-757) were cloned into the EcoRI and SalI sites of the pEGFP-C2 (BD Biosciences Clontech Franklin Lakes, N.J.) expression vector.

### Peptide Library Screening

Phosphoserine and phosphothreonine oriented degenerate peptide libraries consisting of the sequences Gly-Ala-X-X-X-B-(pSer/pThr)-Gln-J-X-X-X-Ala-Lys-Lys-Lys SEQ ID NO: 62, Met-Ala-X-X-X-X-pThr-X-X-X-X-Ala-Lys-Lys-Lys SEQ ID NO: 47, and Met-Ala-X-X-X-XpSer-X-X-X-X-X-Ala-Lys-Lys-Lys SEQ ID NO: 58; where pS is phosphoserine, pT is phosphothreonine; and X denotes all amino acids except Cys. In the (pSer/pThr)-Gln library, B is a biased mixture of the amino acids A, I, L, M, N, P, S, T, V, and J represents a biased mixture of 25% E, 75% X, where X denotes all amino acids except Arg, Cys, His, Lys. Peptides were synthesized using N-α-FMOC-protected amino acids and standard BOP/HOBt coupling chemistry. Peptide library screening was performed using 125 μl of glutathione beads containing saturating amounts of GST-PTIP BRCT or GST-BRCA1 BRCT domains (1-1.5 mg) as described by Yaffe and Cantley (Methods Enzymol 328:157-70, 2000). Beads were packed in a 1 mL column and incubated with 0.45 mg of the peptide library mixture for 10 minutes at room temperature in PBS (150 mM NaCl, 3 mM KCl, 10 mM Na₂HPO₄, 2 mm KH2PO4, pH 7.6). Unbound peptides were removed from the column by two washes with PBS containing 1.0% NP-40 followed by two washes with PBS. Bound peptides were eluted with 30% acetic acid for 10 minutes at room temperature, lyophilized, resuspended in H2O, and sequenced by automated Edman degradation on a PROCISE protein microsequencer (Perkin-Elmer Corporation, Norwalk Conn.). Selectivity values for each amino acid were determined by comparing the relative abundance (mole percentage) of each amino acid at a particular sequencing cycle in the recovered peptides to that of each amino acid in the original peptide library mixture at the same position.

### Isothermal Titration Calorimetry

Peptides were synthesized by solid phase technique with three C-terminal lysines to enhance solubility. The peptides were then purified by reverse phase HPLC following deprotection and confirmed by MALDI-TOF mass spectrometry. Calorimetry measurements were performed using a VP-ITC microcalorimeter (MicroCal Inc., Studio City, Calif.). Experiments involved serial 10 μL injections of peptide solutions (20 μM-150 μM) into a sample cell containing 15 μM GST-PTIP BRCT domains (residues 550-757) or 15 μM GST-BRCA1 BRCT domains (residues 1634-1863) in 50 mM Tris/HCl (pH 8.1), 300 mM NaCl. Twenty injections were performed with 240 second intervals between injections and a reference power of 25 μCal/s. Binding isotherms were plotted and analyzed using ORIGIN Software (MicroCal Inc. Studio City, Calif.).

### Peptide Filter Array

An ABIMED peptide arrayer with a computer controlled Gilson diluter and liquid handling robot (Abimed GmbH, Dusseldorf, Germany) was used to synthesize peptides onto an amino-PEG cellulose membrane using N-α-FMOC-protected amino acids and DIC/HOBT coupling chemistry. The membranes were blocked in 5% milk/TBS-T (0.1%) for lhour at room temperature, incubated with 0.05 μM GST-PTIP BRCT domains (residues 550-757) or GST-BRCA1 BRCT domains (residues 1634-1863) in 5% milk, 50 mM Tris/HCl (pH 7.6), 150 mM NaCl, 2 mM EDTA, 2 mM DTT for 1 hour at room temperature and washed four times with TBS-T (0.1%). The membranes were then incubated with anti-GST conjugated HRP (Amersham) in 5% milk/TBS-T (0.1%) for 1 hour at room temperature, washed five times with TBS-T (0.1%), and subjected to chemiluminescence.

### PTIP BRCT Domains and BRCA1 BRCT Domains Binding to Cellular Substrates

U2OS cells were either treated with 10 Gy of ionizing radiation or mock irradiated and allowed to recover for 30-120 minutes. Cells were subsequently lysed in 50 mM Tris/HCl (pH7.6), 150 mM NaCl, 1.0% NP-40, 5 mM EDTA, 2 mM DTT, 8 μg/mL pepstatin, 8 μg/mL aprotinin, 8 μg/mL leupeptin, 2 mM Na3VO4, 10 mM NaF, 1 μM microcystin. The lysates (0.5-2 mg) were incubated with 20 μL glutathione beads containing 10-20 μg of GST-PTIP BRCT domains (residues 550-757), GST-BRCA1 BRCT domains (residues 1634-1863), or GST for 120 minutes at 4° C. Beads were washed three times with lysis buffer. Precipitated proteins were eluted in sample buffer and detected by blotting with anti-ATM/ATR substrate (pSer/pThr)Gln antibody (CELL SIGNALING TECHNOLOGY, Inc Beverly, Mass.), polyclonal anti-53BP1 (ONCOGENE RESEARCH PRODUCTS, San Diego, Calif. 92121), or monoclonal anti-HA (COVANCE Inc, Princeton, N.J.). For peptide competition experiments, GST-PTIP BRCT domains or GST-BRCA1 BRCT domains were immobilized on glutathionine beads and preincubated with 350 μM of BRCTtide-optimal, 7pT, 7T, pSQ-library, SQ-library, or pTP-library for 1 hour at 4° C. and washed three times with lysis buffer.

### Immunofluorescence and Microscopy

U2OS cells were seeded onto 18 mm2 coverslips and transfected with GFP-PTIP constructs FL (residues 1-757), !BRCT (residues 1-550), or (BRCT)2 (residues 550-757) using FUGENE6 transfection reagent (Roche, Base1, Switzerland) according to manufacture's protocol. Twenty-four hours following transfection, the cells were either treated with 10 Gy of ionizing radiation or mock irradiated and allowed to recover for 120 minutes. Cells were fixed in 3% paraformaldehyde/2% sucrose for 15 minutes at room temperature and extracted with a 0.5% Triton X-100 solution containing 20 mM Tris-HCl (pH 7.8), 75 mM NaCl, 300 mM sucrose, and 3 mM MgC12 for 15 minutes at room temperature. Slides were stained with primary antibodies at 4° C. overnight, then stained with a Texas Red conjugated anti-mouse or anti-rabbit secondary antibody for 60 minutes (Molecular Probes, Eugene, Oreg.) at room temperature. Primary antibodies used were rabbit anti-53BP1 (Oncogene Research Products, San Diego, Calif.), mouse anti-g-H2AX (Upstate, Charlottesville, Va.), and rabbit anti-(pS/pT)Q (Cell Signaling Technology, Inc., Beverly, Mass.). Images were collected on a Deltavision microscope (Carl Zeiss, Thornwood, N.Y.) and digitally deconvolved using SOFTWORX graphics processing software (SGI, CSIF, Stanford, Calif.).

### Peptidomimetics

Peptide derivatives (e.g. peptidomimetics) include cyclic peptides, peptides obtained by substitution of a natural amino acid residue by the corresponding D-stereoisomer, or by a unnatural amino acid residue, chemical derivatives of the peptides, dual peptides, multimers of the peptides, and peptides fused to other proteins or carriers. A cyclic derivative of a peptide of the invention is one having two or more additional amino acid residues suitable for cyclization. These residues are often added at the carboxyl terminus and at the amino terminus. A peptide derivative may have one or more amino acid residues replaced by the corresponding D-amino acid residue. In one example, a peptide or peptide derivative of the invention is all-L, all-D, or a mixed D,L-peptide. In another example, an amino acid residue is replaced by a unnatural amino acid residue. Examples of unnatural or derivatized unnatural amino acids include Na-methyl amino acids, Cα-methyl amino acids, and β-methyl amino acids.

A chemical derivative of a peptide of the invention includes, but is not limited to, a derivative containing additional chemical moieties not normally a part of the peptide. Examples of such derivatives include: (a) N-acyl derivatives of the amino terminal or of another free amino group, where the acyl group may be either an alkanoyl group, e.g., acetyl, hexanoyl, octanoyl, an aroyl group, e.g., benzoyl, or a blocking group such as Fmoc (fluorenylmethyl-O—CO—), carbobenzoxy (benzyl-O—CO—), monomethoxysuccinyl, naphthyl-NH—CO—, acetylamino-caproyl, adamantyl-NH—CO—; (b) esters of the carboxyl terminal or of another free carboxyl or hydroxy groups; (c) amides of the carboxyl terminal or of another free carboxyl groups produced by reaction with ammonia or with a suitable amine; (d) glycosylated derivatives; (e) phosphorylated derivatives; (f) derivatives conjugated to lipophilic moieties, e.g., caproyl, lauryl, stearoyl; and (g) derivatives conjugated to an antibody or other biological ligand. Also included among the chemical derivatives are those derivatives obtained by modification of the peptide bond—CO—NH—, for example, by: (a) reduction to —CH₂—NH—; (b) alkylation to —CO—N(alkyl)—; and (c) inversion to —NH—CO—.

A dual peptide of the invention consists of two of the same, or two different, peptides of the invention covalently linked to one another, either directly or through a spacer.

Multimers of the invention consist of polymer molecules formed from a number of the same or different peptides or derivatives thereof.

In one example, a peptide derivative is more resistant to proteolytic degradation than the corresponding non-derivatized peptide. For example, a peptide derivative having D-amino acid substitution(s) in place of one or more L-amino acid residue(s) resists proteolytic cleavage.

In another example, the peptide derivative has increased permeability across a cell membrane as compared to the corresponding non-derivatized peptide. For example, a peptide derivative may have a lipophilic moiety coupled at the amino terminus and/or carboxyl terminus and/or an internal site. Such derivatives are highly preferred when targeting intracellular protein-protein interactions, provided they retain the desired functional activity.

In another example, a peptide derivative binds with increased affinity to a ligand (e.g., a Polo box domain).

The peptides or peptide derivatives of the invention are obtained by any method of peptide synthesis known to those skilled in the art, including synthetic and recombinant techniques. For example, the peptides or peptide derivatives can be obtained by solid phase peptide synthesis which, in brief, consists of coupling the carboxyl group of the C-terminal amino acid to a resin and successively adding N-alpha protected amino acids. The protecting groups may be any such groups known in the art. Before each new amino acid is added to the growing chain, the protecting group of the previous amino acid added to the chain is removed. The coupling of amino acids to appropriate resins has been described by Rivier et al. (U.S. Pat. No. 4,244,946). Such solid phase syntheses have been described, for example, by Merrifield, J. Am. Chem. Soc. 85:2149, 1964; Vale et al., Science 213:1394-1397, 1984; Marki et al., J. Am. Chem. Soc. 10:3178, 1981, and in U.S. Pat. Nos. 4,305,872 and 4,316,891. In a preferred aspect, an automated peptide synthesizer is employed.

Purification of the synthesized peptides or peptide derivatives is carried out by standard methods, including chromatography (e.g., ion exchange, affinity, and sizing column chromatography), centrifugation, differential solubility, hydrophobicity, or by any other standard technique for the purification of proteins. In one embodiment, thin layer chromatography is employed. In another embodiment, reverse phase HPLC (high performance liquid chromatography) is employed.

Finally, structure-function relationships determined from the peptides, peptide derivatives, and other small molecules of the invention may also be used to prepare analogous molecular structures having similar properties. Thus, the invention is contemplated to include molecules in addition to those expressly disclosed that share the structure, hydrophobicity, charge characteristics and side chain properties of the specific embodiments exemplified herein.

In one example, such derivatives or analogs that have the desired binding activity can be used for binding to a molecule or other target of interest, such as any Polo-box domain. Derivatives or analogs that retain, or alternatively lack or inhibit, a desired property-of-interest (e.g., inhibit PBD binding to a natural ligand), can be used to inhibit the biological activity of a Polo-like kinase (e.g., Plk-1, 2, or 3).

In particular, peptide derivatives are made by altering amino acid sequences by substitutions, additions, or deletions that provide for functionally equivalent molecules, or for functionally enhanced or diminished molecules, as desired. Due to the degeneracy of the genetic code, other nucleic acid sequences that encode substantially the same amino acid sequence may be used for the production of recombinant peptides. These include, but are not limited to, nucleotide sequences comprising all or portions of a peptide of the invention that is altered by the substitution of different codons that encode a functionally equivalent amino acid residue within the sequence, thus producing a silent change.

The derivatives and analogs of the invention can be produced by various methods known in the art. The manipulations that result in their production can occur at the gene or protein level. For example, a cloned nucleic acid sequence can be modified by any of numerous strategies known in the art (Sambrook et al., 1989, Molecular Cloning, A Laboratory Manual, 2d ed., Cold Spring Harbor Laboratory Press, Cold Spring Harbor, N.Y.). The sequence can be cleaved at appropriate sites with restriction endonuclease(s), followed by further enzymatic modification if desired, isolated, and ligated in vitro.

### Modified Phosphopeptides

A phosphopeptide of the invention may include, but it is not limited to, an unnatural N-terminal amino acid of the formula (III):

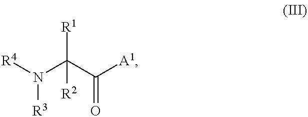

where A¹ is an amino acid or peptide chain linked via an α-amino group; R¹ and R³ are independently hydrogen, C₁₋₅ branched or linear C₁₋₅ alkyl, C₁₋₅ alkaryl, heteroaryl, and aryl, each of which are unsubstituted or substituted with a substitutent selected from: 1 to 3 of C₁₋₅ alkyl, 1 to 3 of halogen, 1 to 2 of —OR⁵, N(R⁵)(R⁶), SR⁵, N—C(NR⁵)NR⁶R⁷, methylenedioxy, —S(O)ₘR⁵, 1 to 2 of —CF₃, —OCF₃, nitro, —N(R⁵)C(O)(R⁶), —C(O)OR⁵, —C(O)N(R⁵)(R⁶), -1H-tetrazol-5-yl, —SO₂N(R⁵)(R⁶), —N(R⁵)SO₂ aryl, or —N(R⁵)SO₂R⁶; R⁵, R⁶ and R⁷ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl; R² is hydrogen, F, C₁₋₅ linear or branched alkyl, C₁₋₅ alkaryl; or R² and R¹ are joined to form a C₃₋₈ cyclic ring, optionally including oxygen, sulfur, or NR⁷, where R⁷ is hydrogen, or C₁₋₅ alkyl, optionally substituted by hydroxyl, or R² and R³ are joined to form a C₃₋₈ cyclic ring, optionally substituted by hydroxyl and optionally including oxygen, sulfur or NR⁷, where R⁷ is hydrogen, or C₁₋₅ alkyl; R² is hydrogen, F, C₁₋₅ linear or branched alkyl, C₁₋₅ alkaryl; and R⁴ is hydrogen, C₁₋₅ branched or linear C₁₋₅ alkyl, C₁₋₅ alkaryl, heteroaryl, and aryl, each of which are unsubstituted or substituted with a substitutent selected from: 1 to 3 of C₁₋₅ alkyl, 1 to 3 of halogen, 1 to 2 of —OR⁵, N(R⁵)(R⁶), N—C(NR⁵)NR⁶R⁷, methylenedioxy, —S(O)ₘR⁵ (where m is 0-2), 1 to 2 of —CF₃, —OCF₃, nitro, —N(R⁵)C(O)(R⁶), —N(R⁵)C(O)(OR⁶), —C(O)OR⁵, —C(O)N(R⁵)(R⁶), -1H-tetrazol-5-yl, —SO₂N(R⁵)(R⁶), —N(R⁵)SO₂ aryl, or —N(R⁵)SO₂R⁶, R⁵, R⁶ and R⁷ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl.

The phosphopeptides of the invention may also include an internal unnatural internal amino acid of the formula:

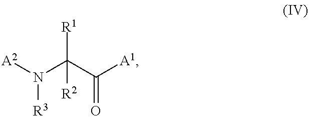

where A² is an amino acid or peptide chain linked via an α-carboxy group; A¹ is an amino acid or peptide chain linked via an α-amino group; R¹ and R³ are independently hydrogen, C₁₋₅ branched or linear C₁₋₅ alkyl, C₁₋₅ alkaryl, heteroaryl, and aryl, each of which are unsubstituted or substituted with a substitutent selected from: 1 to 3 of C₁₋₅ alkyl, 1 to 3 of halogen, 1 to 2 of —OR⁵, N(R⁵)(R⁶), SR⁵, N—C(NR⁵)NR⁶R⁷, methylenedioxy, —S(O)ₘR⁵ (m is 1-2), 1 to 2 of —CF₃, —OCF₃, nitro, —N(R⁵)C(O)(R⁶), —C(O)OR⁵, —C(O)N(R⁵)(R⁶), -1H-tetrazol-5-yl, —SO₂N(R⁵)(R⁶), —N(R⁵)SO₂ aryl, or —N(R⁵)SO₂R⁶; R⁵, R⁶ and R⁷ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl; and R² is hydrogen, F, C₁₋₅ linear or branched alkyl, C₁₋₅ alkaryl; or R² and R¹ are joined to form a C₃₋₈ cyclic ring, optionally including oxygen, sulfur or NR⁷, where R⁷ is hydrogen, or C₁₋₅ alkyl, optionally substituted by hydroxyl, or R² and R³ are joined to form a C₃₋₈ cyclic ring, optionally substituted by hydroxyl and optionally including oxygen, sulfur or NR⁷, where R⁷ is hydrogen, or C₁₋₅ alkyl.

The invention also includes modifications of the phosphopeptides of the invention, wherein an internal unnatural internal amino acid of the formula:

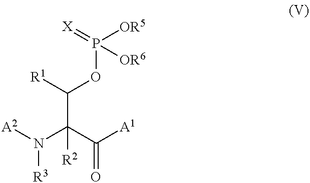

is present, where A² is an amino acid or peptide chain linked via an α-carboxy group; A¹ is an amino acid or peptide chain linked via an α-amino group; R¹ and R³ are independently hydrogen, C₁₋₅ branched or linear C₁₋₅ alkyl, and C₁₋₅ alkaryl; R² is hydrogen, F, C₁₋₅ linear or branched alkyl, C₁₋₅ alkaryl; or R² and R¹ are joined to form a C₃₋₈ cyclic ring, optionally including oxygen, sulfur or NR⁷, where R⁷ is hydrogen, or C₁₋₅ alkyl, optionally substituted by hydroxyl; X is O or S; and R⁵ and R⁶ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl; or R⁵ and R⁶ are joined to form a C₃₋₈ cyclic ring, optionally including oxygen, sulfur or NR⁷, where R⁷ is hydrogen, or C₁₋₅ alkyl, optionally substituted by hydroxyl.

The phosphopeptides of the invention may also include a C-terminal unnatural internal amino acid of the formula:

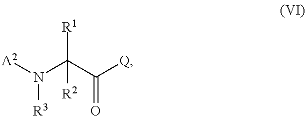

where A² is an amino acid or peptide chain linked via an α-carboxy group; R¹ and R³ are independently hydrogen, C₁₋₅ branched or linear C₁₋₅ alkyl, C₁₋₅ alkaryl, heteroaryl, and aryl, each of which are unsubstituted or substituted with a substitutent selected from: 1 to 3 of C₁₋₅ alkyl, 1 to 3 of halogen, 1 to 2 of —OR⁵, N(R⁵)(R⁶), SR^{S}, N—C(NR⁵)NR⁶R⁷, methylenedioxy, —S(O)ₘR⁵, 1 to 2 of —CF₃, -OCF₃, nitro, —N(R⁵)C(O)(R⁶), —C(O)OR⁵, —C(O)N(R⁵)(R⁶), -1H-tetrazol-5-yl, —SO₂N(R⁵)(R⁶), —N(R⁵)SO₂ aryl, or —N(R⁵)SO₂R⁶; R⁵, R⁶ and R⁷ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl; R² is hydrogen, F, C₁₋₅ linear or branched alkyl, C₁₋₅ alkaryl; or R² and R¹ are joined to form a C₃₋₈ cyclic ring, optionally including oxygen, sulfur or NR⁷, where R⁷ is hydrogen, or C₁₋₅ alkyl, optionally substituted by hydroxyl; or R² and R³ are joined to form a C₃₋₈ cyclic ring, optionally substituted by hydroxyl and optionally including oxygen, sulfur or NR⁷, where R⁷ is hydrogen, or C₁₋₅ alkyl; R² is hydrogen, F, C₁₋₅ linear or branched alkyl, C₁₋₅ alkaryl; and Q is OH, OR^{S}, or NR⁵R⁶, where R⁵, R⁶ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl. Methods well known in the art for modifying peptides are found, for example, in “Remington: The Science and Practice of Pharmacy” (20th ed., ed. A. R. Gennaro, 2000, Lippincott Williams & Wilkins, Philadelphia).

### Therapeutic Uses

Peptide Synthesis and Conjugation

Phosphopeptides of the invention are prepared as detailed above. Alternatively, phosphopeptides can be prepared using standard FMOC chemistry on 2-chlorotrityl chloride resin (Int. J. Pept. Prot. Res. 38, 1991, 555-61). Cleavage from the resin is performed using 20% acetic acid in dichloromehane (DCM), which leaves the side chain still blocked. Free terminal carboxylate peptide is then coupled to 4′(aminomethy)-fluorescein (Molecular Probes, A-1351; Eugene, Oreg.) using excess diisopropylcarbodiimide (DIC) in dimethylformamide (DMF) at room temperature. The fluorescent N—C blocked peptide is purified by silica gel chromatography (10% methanol in DCM). The N terminal FMOC group is then removed using piperidine (20%) in DMF, and the N-free peptide, purified by silica gel chromatography (20% methanol in DCM, 0.5% HOAc). Finally, any t-butyl side chain protective groups are removed using 95% trifluoroacetic acid containing 2.5% water and 2.5% triisopropyl silane. The peptide obtained in such a manner should give a single peak by HPLC and is sufficiently pure for carrying on with the assay described below.

Phosphopeptide Modifications

It is understood that modifications can be made to the amino acid residues of the phosphopeptides of the invention, to enhance or prolong the therapeutic efficacy and/or bioavailability of the phosphopeptide. Accordingly, α-amino acids having the following general formula (I):

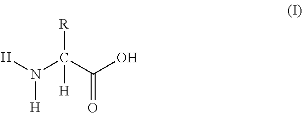

where R defines the specific amino acid residue, may undergo various modifications. Exemplary modifications of α-amino acids, include, but are not limited to, the following formula (II):

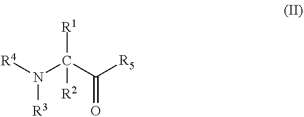

R₁, R₂, R₃, R₄, and R₅, are independently hydrogen, hydroxy, nitro, halo, C₁₋₅ branched or linear alkyl, C₁₋₅ alkaryl, heteroaryl, and aryl; wherein the alkyl, alkaryl, heteroaryl, and aryl may be unsubstituted or substituted by one or more substituents selected from the group consisting of C₁₋₅ alkyl, hydroxy, halo, nitro, C₁₋₅ alkoxy, C₁₋₅ alkylthio, trihalomethyl, C₁₋₅ acyl, arylcarbonyl, heteroarylcarbonyl, nitrile, C₁₋₅ alkoxycarbonyl, oxo, arylalkyl (wherein the alkyl group has from 1 to 5 carbon atoms) and heteroarylalkyl (wherein the alkyl group has from 1 to 5-carbon atoms); alternatively, R₁ and R₂ are joined to form a C₃₋₈ cyclic ring, optionally including oxygen, sulfur or hydrogen, or C₁₋₅ alkyl, optionally substituted by hydroxyl; or R₂ and R₃ are joined to form a C₃₋₈ cyclic ring, optionally substituted by hydroxyl and optionally including oxygen, sulfur, C₁₋₅ aminoalkyl, or C₁₋₅ alkyl. Methods well known in the art for making modifications are found, for example, in “Remington: The Science and Practice of Pharmacy” (20th ed., ed. A. R. Gennaro, 2000, Lippincott Williams & Wilkins), hereby incorporated by reference.

Assays and High Throughput Assays

Fluorescence polarization assays can be used in displacement assays to identify small molecule peptidomimetics. The following is an exemplary method for use of fluorescence polarization, and should not be viewed as limiting in any way. For screening, all reagents are diluted at the appropriate concentration and the working solution, kept on ice. The working stock concentration for GST and GST fusion proteins are ˜4 ng/μL, Fluorescein-labeled phosphopeptides can be used at a concentration of 1.56 fmol/μL, while cold phosphopeptides and peptides at 25 pmol/μL. Samples are incubated at a total volume of 200 μL per well in black flat bottom plates, Biocoat, #359135 low binding (BD BioSciences; Bedford, Mass.). Assays are started with the successive addition using a Labsystem Multi-prop 96/384 device (Labsystem; Franklin, Mass.) of 50 μL test compounds, diluted in 10% DMSO (average concentration of 28 μM), 50 μL of 50 mM MES-pH 6.5, 50 μL of Fluorescein-phosphopeptide, 50 μL of GST-Plk-1 PBD, 50 μL of unlabeled phosphopeptide, or unphosphorylated peptide can be used as a negative control. Once added, all the plates are placed at 4° C. Following overnight incubation at 4° C., the fluorescence polarization is measured using a Polarion plate reader (Tecan, Research Triangle Park, N.C.). A xenon flash lamp equipped with an excitation filter of 485 nm and an emission filter of 535 nm. The number of flashes is set at 30. Raw data can then be converted into a percentage of total interaction(s). All further analysis can be performed using SPOTFIRE data analysis software (SPOTFIRE, Somerville, Mass.)

Upon selection of active compounds, auto-fluorescence of the hits is measured as well as the fluorescein quenching effect, where a measurement of 2000 or more units indicates auto-fluorescence, while a measurement of 50 units indicates a quenching effect. Confirmed hits can then be analyzed in dose-response curves (IC₅₀) for reconfirmation. Best hits in dose—response curves can then be assessed by isothermal titration calorimetry using GST-Plk-1 PBD.

Alternate Binding and Displacement Assays

Fluorescence polarization assays are but one means to measure phosphopeptide-protein interactions in a screening strategy. Alternate methods for measuring phosphopeptide-protein interactions are known to the skilled artisan. Such methods include, but are not limited to mass spectrometry (Nelson and Krone, J. Mol. Recognit., 12:77-93, 1999), surface plasmon resonance (Spiga et al., FEBS Lett., 511:33-35, 2002; Rich and Mizka, J. Mol. Recognit., 14:223-8, 2001; Abrantes et al., Anal. Chem., 73:2828-35, 2001), fluorescence resonance energy transfer (FRET) (Bader et al., J. Biomol. Screen, 6:255-64, 2001; Song et al., Anal. Biochem. 291:133-41, 2001; Brockhoff et al., Cytometry, 44:338-48, 2001), bioluminescence resonance energy transfer (BRET) (Angers et al., Proc. Natl. Acad. Sci. USA, 97:3684-9, 2000; Xu et al., Proc. Natl. Acad. Sci. USA, 96:151-6, 1999), fluorescence quenching (Engelborghs, Spectrochim. Acta A. Mol. Biomol. Spectrosc., 57:2255-70, 70; Geoghegan et al., Bioconjug. Chem. 11:71-7, 2000), fluorescence activated cell scanning/sorting (Barth et al., J. Mol. Biol., 301:751-7, 2000), ELISA, and radioimmunoassay (RIA).

Test Extracts and Compounds

In general, peptidomimetic compounds that affect phosphopeptide-protein interactions are identified from large libraries of both natural products, synthetic (or semi-synthetic) extracts or chemical libraries, according to methods known in the art.

Those skilled in the art will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from, for example, Brandon Associates (Merrimack, N.H.) and Aldrich Chemical (Milwaukee, Wis.)

Alternatively, libraries of natural compounds in the form of bacterial, fungal, plant, and animal extracts are commercially available from a number of sources, including, but not limited to, Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art (e.g., by combinatorial chemistry methods or standard extraction and fractionation methods). Furthermore, if desired, any library or compound may be readily modified using standard chemical, physical, or biochemical methods.

Administration of Phosphopeptides and Peptidomimetic Small Molecules

By selectively disrupting or preventing a phosphoprotein from binding to its natural partner(s) through its binding site, the phosphopeptides of the invention, or derivatives, or peptidomimetics thereof, can significantly alter the biological activity or the biological function of a polo-like kinase. Therefore, the phosphopeptides, or derivatives thereof, of the invention can be used for the treatment of a disease or disorder characterized by inappropriate cell cycle regulation or apoptosis.

Diseases or disorders characterized by inappropriate cell cycle regulation, include hyperproliferative disorders, such as neoplasias. Examples of neoplasms include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenriglioma, schwannoma, meningioma, melanoma, neuroblastoma, and retinoblastoma).

Cells undergoing inappropriate apoptosis include neurons in a patient who has a neurodegenerative disease (e.g., Parkinson's disease, Alzheimer's disease, or stroke), and cardiomyocytes (e.g., after myocardial infarction or over the course of congestive heart failure). Compositions of the invention, i.e., inhibitors of Plk-3, may be useful in treating a cell undergoing inappropriate apoptosis.

A Plk-1 PBD-binding phosphopeptide or peptidomimetic small molecule may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

The pharmaceutical compositions of the present invention are prepared in a manner known per se, for example by means of conventional dissolving, lyophilising, mixing, granulating or confectioning processes. Methods well known in the art for making formulations are found, for example, in “Remington: The Science and Practice of Pharmacy” (20th ed., ed. A. R. Gennaro, 2000, Lippincott Williams & Wilkins, Philadelphia).

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilisers, wetting and/or emulsifying agents, solubilisers, salts for regulating the osmotic pressure and/or buffers, and are prepared in a manner known per se, for example by means of conventional dissolving or lyophilising processes. The said solutions or suspensions may comprise viscosity-increasing substances, such as sodium carboxymethylcellulose, carboxymethylcellulose, dextran, poly vinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of anti oxidants, for example, vitamins E, β-carotene, or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are there fore to be mentioned: ethyl oleate, isopropyl myristate, isopropyl palmitate, “Labrafil M 2375” (poly oxyethylene glycerol trioleate, Gattefoss, Paris), “Miglyol 812” (triglyceride of saturated fatty acids with a chain length of C_{g} to C₁₂, Huls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, drage cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinyl-pyrrolidone, and/or, if desired, disintegrates, such as the above-mentioned starches, also carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Drage cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticiser, such as glycerol or sorbitol. The dry-filled capsules may comprise the active ingredient in the form of granules, for example with fillers, such as lactose, binders, such as starches, and/or glidants, such as talc or magnesium stearate, and if desired with stabilisers. In soft capsules the active ingredient is preferably dissolved or suspended in suitable oily excipients, such as fatty oils, paraffin oil or liquid polyethylene glycols, it being possible also for stabilisers and/or antibacterial agents to be added. Dyes or pigments may be added to the tablets or drage coatings or the capsule casings, for example for identification purposes or to indicate different doses of active ingredient.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, drages, tablets or capsules.

The formulations can be administered to human patients in a therapeutically effective amount (e.g., an amount that decreases, suppresses, attenuates, diminishes, arrests, or stabilizes the development or progression of a disease, disorder, or infection in a eukaryotic host organism). The preferred dosage of therapeutic agent to be administered is likely to depend on such variables as the type and extent of the disorder, the overall health status of the particular patient, the formulation of the compound excipients, and its route of administration.

For any of the methods of application described above, a Plk-1 PBD-interacting small molecule may be applied to the site of the needed therapeutic event (for example, by injection), or to tissue in the vicinity of the predicted therapeutic event or to a blood vessel supplying the cells predicted to require enhanced therapy.

The dosages of Plk-1 PBD-interacting small molecule(s) depends on a number of factors, including the size and health of the individual patient, but, generally, between 0.1 mg and 1000 mg inclusive are administered per day to an adult in any pharmaceutically acceptable formulation. In addition, treatment by any of the approaches described herein may be combined with more traditional therapies.

### Combination Therapy

If desired, treatment with Plk-1 PBD-interacting small molecule may be combined with more traditional therapies for the proliferative disease such as surgery or administration of chemotherapeutics or other anti-cancer agents, including, for example, γ-radiation, alkylating agents (e.g., nitrogen mustards such as cyclophosphamide, ifosfamide, trofosfamide, and chlorambucil; nitrosoureas such as carmustine, and lomustine; alkylsulphonates such as bisulfan and treosulfan; triazenes such as dacarbazine; platinum-containing compounds such as cisplatin and carboplatin), plant alkaloids (e.g., vincristine, vinblastine, anhydrovinblastine, vindesine, vinorelbine, paclitaxel, and docetaxol), DNA topoisomerase inhibitors (e.g., etoposide, teniposide, topotecan, 9-aminocamptothecin, (campto) irinotecan, and crisnatol), mytomycins (e.g., mytomicin C), antifolates (e.g., methotrexate, trimetrexate, mycophenolic acid, tiazofurin, ribavirin, EICAR, hydroxyurea, and deferoxamine), uracil analogs (5-fluorouracil, floxuridine, doxifluridine, and ratitrexed), cytosine analogs (cytarbine, cytosine arabinoside, and fludarabine), purine analogs (e.g., mercaptopurine, and thioguanine), hormonal therapies (e.g., tamoxifen, raloxifene, megestrol, goserelin, leuprolide acetate, flutamide, and bicalutamide), vitamin D3 analogs (EB 1089, CB 1093, and KH 1060), vertoporfin, phthalocyanine, photosensitizer Pc4, demethoxy-hypocrellin A, interferon-α, interferon-γ, tumor necrosis factor, lovastatin, 1-methyl-4-phenylpyridinium ion, staurosporine, actinomycin D, dactinomycin, bleomycin A2, bleomycin B2, adriamycin, peplomycin, daunorubican, idarubican, epirubican, pirarubican, zorubican, mitoxantrone, and verapamil.

### Other Embodiments

From the foregoing description, it is apparent that variations and modifications may be made to the invention described herein to adopt it to various usages and conditions. Such embodiments are also within the scope of the following claims.

All patents and publications mentioned in this specification are hereby incorporated by reference to the same extent as if each independent publication or patent application, including 60/426,132, was specifically and individually indicated to be incorporated by reference.

What is claimed is:

1. A method for displaying a three-dimensional model of a Polo-box domain of a Plk-1 Polo-like kinase, said method comprising:
(i) providing structural coordinates of said Polo-box domain sufficient for modeling binding of a candidate compound to the phosphopeptide binding pocket of said Polo-box domain, said structural coordinates comprising at least one set of x, y, and z atomic coordinates from Table 5, or a mathematical modification of Table 5 that preserves the relative three-dimensional relationship among the coordinates of Table 5, from a given atom of each of residues His-538, Lys-540, Trp-414, and Leu-491 of said Polo-box domain, or a set of x, y, and z atomic coordinates that have a root mean square deviation of said set of x, y, and z atomic coordinates of said Polo-box domain of less than 3 Å;
(ii) generating a three-dimensional model of said Polo-box domain utilizing said coordinates of (i); and
(iii) outputting a representation of said three-dimensional model of said Polo-box domain to a display.

2. The method of claim 1, said structural coordinates comprising at least two sets of x, y, and z atomic coordinates from Table 5, or a mathematical modification of Table 5 that preserves the relative three-dimensional relationship among the coordinates of Table 5, from atoms of each of residues His-538, Lys-540, Trp-414, and Leu-491 of said Polo-box domain, or a set of x, y, and z atomic coordinates of said Polo-box domain of less than 3 Å.

3. The method of claim 1, said structural coordinates comprising at least three sets of x, y, and z atomic coordinates from Table 5, or a mathematical modification of Table 5 that preserves the relative three-dimensional relationship among the coordinates of Table 5, from atoms of each of residues His-538, Lys-540, Trp-414, and Leu-491 of said Polo-box domain, or a set of x, y, and z atomic coordinates of said Polo-box domain of less than 3 Å.

4. The method of claim 1, said structural coordinates comprising at least four sets of x, y, and z atomic coordinates from Table 5, or a mathematical modification of Table 5 that preserves the relative three-dimensional relationship among the coordinates of Table 5, from atoms of each of residues His-538, Lys-540, Trp-414, and Leu-491 of said Polo-box domain, or a set of x, y, and z atomic coordinates of said Polo-box domain of less than 3 Å.

5. The method of claim 1, said structural coordinates comprising at least five sets of x, y, and z atomic coordinates from Table 5, or a mathematical modification of Table 5 that preserves the relative three-dimensional relationship among the coordinates of Table 5, from atoms of each of residues His-538, Lys-540, Trp-414, and Leu-491 of said Polo-box domain, or a set of x, y, and z atomic coordinates of said Polo-box domain of less than 3 Å.

6. The method of claim 1, wherein said root mean square deviation is less than 2 Å.

7. The method of claim 1, wherein said root mean square deviation is less than 1 Å.

8. The method of claim 1, wherein said candidate compound is a peptidomimetic.

9. A method of identifying a compound that binds to the phosphopeptide binding pocket of a Polo-box domain of a Plk-1 Polo-like kinase, said method comprising:
(i) providing structural coordinates of said phosphopeptide binding pocket of said Polo-box domain sufficient for modeling binding of a candidate compound to said phosphopeptide binding pocket of said Polo-box domain, said structural coordinates comprising at least one set of x, y, and z atomic coordinates from Table 5, or a mathematical modification of Table 5 that preserves the relative three-dimensional relationship among the coordinates of Table 5, from a given atom of each of residues His-538, Lys-540, Trp-414, and Leu-491 of said Polo-box domain, or a set of x, y, and z atomic coordinates that have a root mean square deviation of said set of x, y, and z atomic coordinates of said Polo-box domain of less than 3 Å;
(ii) employing computational means to perform a computer fitting operation between said structural coordinates of said phosphopeptide binding pocket of said Polo-box domain and a computer model of said candidate compound; and
(iii) evaluating an interaction between said structural coordinates of said phosphopeptide binding pocket of said Polo-box domain and said computer model of said candidate compound to determine the binding affinity between said phosphopeptide binding pocket and said candidate compound,
wherein a binding affinity greater than a predetermined reference value identifies said candidate compound as a compound that binds to said phosphopeptide binding pocket of said Polo-box domain.

10. The method of claim 9, further comprising outputting a representation of a three-dimensional model of said interaction between said phosphopeptide binding pocket and said computer model of said candidate compound to a display.

11. The method of claim 9, further comprising synthesizing said candidate compound.

12. The method of claim 9, further comprising assaying the binding of said Polo-box domain of said Plk-1 Polo-like kinase to a phosphopeptide in the presence of said candidate compound, said method comprising the steps of:
(i) contacting said phosphopeptide and said Polo-box domain to form a complex between said phosphopeptide and said Polo-box domain;
(ii) contacting said complex with said candidate compound; and
(iii) measuring the displacement of said phosphopeptide from said Polo-box domain,
wherein said displacement of said phosphopeptide from said Polo-box domain indicates that said candidate compound inhibits binding of said phosphopeptide to said Polo-box domain.

13. The method of claim 9, further comprising assaying the binding of said Polo-box domain of said Plk-1 Polo-like kinase to a phosphopeptide in the presence of said candidate compound, said method comprising the steps of:
(i) contacting said phosphopeptide and said Polo-box domain in the presence of said candidate compound; and
(ii) measuring binding of said phosphopeptide to said Polo-box domain,
wherein a reduction in the amount of binding of said phosphopeptide to said Polo-box domain in the presence of said candidate compound relative to the amount of binding of said phosphopeptide to said Polo-box domain in the absence of said candidate compound indicates that said candidate compound inhibits binding of said phosphopeptide to said Polo-box domain.

14. The method of claim 9, wherein said candidate compound is a peptidomimetic.

15. The method of claim 9, said structural coordinates comprising at least two sets of x, y, and z atomic coordinates from Table 5, or a mathematical modification of Table 5 that preserves the relative three-dimensional relationship among the coordinates of Table 5, from atoms of each of residues His-538, Lys-540, Trp-414, and Leu-491 of said Polo-box domain, or a set of x, y, and z atomic coordinates of said Polo-box domain of less than 3 Å.

16. The method of claim 9, said structural coordinates comprising at least three sets of x, y, and z atomic coordinates from Table 5, or a mathematical modification of Table 5 that preserves the relative three-dimensional relationship among the coordinates of Table 5, from atoms of each of residues His-538, Lys-540, Trp-414, and Leu-491 of said Polo-box domain, or a set of x, y, and z atomic coordinates of said Polo-box domain of less than 3 Å.

17. The method of claim 9, said structural coordinates comprising at least four sets of x, y, and z atomic coordinates from Table 5, or a mathematical modification of Table 5 that preserves the relative three-dimensional relationship among the coordinates of Table 5, from atoms of each of residues His-538, Lys-540, Trp-414, and Leu-491 of said Polo-box domain, or a set of x, y, and z atomic coordinates of said Polo-box domain of less than 3 Å.

18. The method of claim 9, said structural coordinates comprising at least five sets of x, y, and z atomic coordinates from Table 5, or a mathematical modification of Table 5 that preserves the relative three-dimensional relationship among the coordinates of Table 5, from atoms of each of residues His-538, Lys-540, Trp-414, and Leu-491 of said Polo-box domain, or a set of x, y, and z atomic coordinates of said Polo-box domain of less than 3 Å.

19. The method of claim 9, wherein said root mean square deviation is less than 2 Å.

20. The method of claim 9, wherein said root mean square deviation is less than 1 Å.


SEQUENCE LISTING (129 sequences)

SEQ ID NO: 1 - protein, 603 aa, Homo sapiens
MSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 2 - protein, 7 aa, Artificial Sequence
  Description of Artificial Sequence: Synthetic peptide
  MOD_RES (1)..(1): Pro or Phe
  MOD_RES (2)..(2): Pro or a hydrophobic amino acid
  MOD_RES (3)..(3): Ala, Gln or a hydrophobic amino acid
  MOD_RES (4)..(4): Thr, Gln, His or Met
  MOD_RES (6)..(6): Phosphorylated Thr or phosphorylated Ser
  MOD_RES (7)..(7): Pro or any amino acid
XXXXSXX

SEQ ID NO: 3 - protein, 15 aa, Artificial Sequence
  Description of Artificial Sequence: Synthetic peptide
  MISC_FEATURE (8)..(8): Phosphorylation
MAGPMQSTPLNGAKK

SEQ ID NO: 4 - protein, 685 aa, Homo sapiens
MELLRTITYQPAASTKMCEQALGKGCGADSKKKRPPQPPEESQPPQSQAQVPPAAPHHHHHHSHSGPEISRIIVDPTTGKRYCRGKVLGKGGFAKCYEMTDLTNNKVYAAKIIPHSRVAKPHQREKIDKEIELHRILHHKHVVQFYHYFEDKENIYILLEYCSRRSMAHILKARKVLTEPEVRYYLRQIVSGLKYLHEQEILHRDLKLGNFFINEAMELKVGDFGLAARLEPLEHRRRTICGTPNYLSPEVLNKQGHGCESDIWALGCVMYTMLLGRPPFETTNLKETYRCIREARYTMPSSLLAPAKHLIASMLSKNPEDRPSLDDIIRHDFFLQGFTPDRLSSSCCHTVPDFHLSSPAKNFFKKAAAALFGGKKDKARYIDTHNRVSKEDEDIYKLRHDLKKTSITQQPSKHRTDEELQPPTTTVARSGTPAVENKQQIGDAIRMIVRGTLGSCSSSSECLEDSTMGSVADTVARVLRGCLENMPEADCIPKEQLSTSFQWVTKWVDYSNKYGFGYQLSDHTVGVLFNNGAHMSLLPDKKTVHYYAELGQCSVFPATDAPEQFISQVTVLKYFSHYMEENLMDGGDLPSVTDIRRPRLYLLQWLKSDKALMMLFNDGTFQVNFYHDHTKIIICSQNEEYLLTYINEDRISTTFRLTTLLMSGCSSELKNRMEYALNMLLQRCN

SEQ ID NO: 5 - protein, 646 aa, Homo sapiens
MEPAAGFLSPRPFQRAAAAPAPPAGPGPPPSALRGPELEMLAGLPTSDPGRLITDPRSGRTYLKGRLLGKGGFARCYEATDTETGSAYAVKVIPQSRVAKPHQREKILNEIELHRDLQHRHIVRFSHHFEDADNIYIFLELCSRKSLAHIWKARHTLLEPEVRYYLRQILSGLKYLHQRGILHRDLKLGNFFITENMELKVGDFGLAARLEPPEQRKKTICGTPNYVAPEVLLRQGHGPEADVWSLGCVMYTLLCGSPPFETADLKETYRCIKQVHYTLPASLSLPARQLLAAILRASPRDRPSIDQILRHDFFTKGYTPDRLPISSCVTVPDLTPPNPARSLFAKVTKSLFGRKKKSKNHAQERDEVSGLVSGLMRTSVGHQDARPEAPAASGPAPVSLVETAPEDSSPRGTLASSGDGFEEGLTVATVVESALCALRNCIAFMPPAEQNPAPLAQPEPLVWVSKWVDYSNKFGFGYQLSSRRVAVLFNDGTHMALSANRKTVHYNPTSTKHFSFSVGAVPRALQPQLGILRYFASYMEQHLMKGGDLPSVEEVEVPAPPLLLQWVKTDQALLMLFSDGTVQVNFYGDHTKLILSGWEPLLVTFVARNRSACTYLASHLRQLGCSPDLRQRLRYALRLLRDRSPA

SEQ ID NO: 6 - protein, 7 aa, Artificial Sequence
  Description of Artificial Sequence: Synthetic peptide
  MISC_FEATURE (5)..(5): Phosphorylation
PMQSTPL

SEQ ID NO: 7 - protein, 4 aa, Artificial Sequence
  Description of Artificial Sequence: Synthetic peptide
  MOD_RES (1)..(1): Met, Tyr, Phe, Ile, Leu, His or Lys
  MOD_RES (2)..(2): Ala, His, Met, Thr, Phe or Gln
  MOD_RES (3)..(3): Ser, Ala, Gly or Thr
  MOD_RES (4)..(4): Phosphorylated Ser or phosphorylated Thr
XXXX

SEQ ID NO: 8 - protein, 7 aa, Artificial Sequence
  Description of Artificial Sequence: Synthetic peptide
  MOD_RES (1)..(1): Any amino acid
  MOD_RES (2)..(2): Met, Tyr, Phe, Ile, Leu, His or Lys
  MOD_RES (3)..(3): Ala, His, Met, Thr, Phe or Gln
  MOD_RES (4)..(4): Ser, Ala, Gly or Thr
  MOD_RES (5)..(5): Phosphorylated Ser or phosphorylated Thr
  MOD_RES (6)..(6): Pro, Met or Asn
  MOD_RES (7)..(7): Any amino acid
XXXXXXX

SEQ ID NO: 9 - protein, 6 aa, Artificial Sequence
  Description of Artificial Sequence: Synthetic peptide
  MOD_RES (4)..(4): Phosphorylated Thr or phosphorylated Ser
MQSXPL

SEQ ID NO: 10 - protein, 9 aa, Artificial Sequence
  Description of Artificial Sequence: Synthetic peptide
  MOD_RES (4)..(4): Phosphorylated Ser or phosphorylated Thr
YDIXQVFPF

SEQ ID NO: 11 - DNA, 82899 bp, Homo sapiens
attggctcttaccacttgtccctcaaaacgaccaccccattgactggtggcgattgcgtcgacggagacggggcaaaagcaagctgaacccgaaaaataacaaacactggggctgaggggtggaactacgagtgcgcagacatgggccagagcgcatttcccctgccccaggcaaattcggcgctcactgcgtccccgcaggccactgaccttacaagactacttgccccagactcctggggctggatgggaattgtagtctccctaaagagttgtacgtatctttttaaggcctagtttctgctttcaaaatacgaaaacataacaacactccagtccataactgttgacaagtacaagcgcgcacaggtctccaatctatccactggatttccgtgagaattgtgcccgctctggtattggatgttcctctccataagactacagtttctaaggaacactgtggcgaagacctttcattccgcaacgcatgctggaaataattatttccctccacccccccaacaatccttattacttatatttaccgaaactggagacctccattagggcggaaagagtgggggattgggacctcttcttacgactgctttggacaataggtagcgattctgaccttcgtacagcaattactgtgatgcaataagccgcaactggaagagtagaggctagagggcaggcactttatggcaaactcaggtagaattcttcctcttccgtctctttccttttacgtcatccgggggcagactgggtggccaatccagagccccgagagacgcttggctctttctgtccctcccatcctctgattgtaccttgatttcgtattctgagaggctgctgcttagcggtagccccttggtttccgtggcaacggaaaagcgcgggaattacagataaattaaaactgcgactgcgcggcgtgagctcgctgagacttcctggacgggggacaggctgtggggtttctcagataactgggcccctgcgctcaggaggccttcaccctctgctctgggtaaaggtagtagagtcccgggaaagggacagggggcccaagtgatgctctggggtactggcgtgggagagtggatttccgaagctgacagatgggtattctttgacggggggtaggggcggaacctgagaggcgtaaggcgttgtgaaccctggggaggggggcagtttgtaggtcgcgagggaagcgctgaggatcaggaagggggcactgagtgtccgtgggggaatcctcgtgataggaactggaatatgccttgagggggacactatgtctttaaaaacgtcggctggtcatgaggtcaggagttccagaccagcctgaccaacgtggtgaaactccgtctctactaaaaatacaaaaattagccgggcgtggtgccgctccagctactcaggaggctgaggcaggagaatcgctagaacccgggaggcggaggttgcagtgagccgagatcgcgccattgcactccagcctgggcgacagagcgagactgtctcaaaacaaaacaaaacaaaacaaaacaaaaaacaccggctggtatgtatgagaggatgggaccttgtggaagaagaggtgccaggaatatgtctgggaaggggaggagacaggattttgtgggagggagaacttaagaactggatccatttgcgccattgagaaagcgcaagagggaagtagaggagcgtcagtagtaacagatgctgccggcagggatgtgcttgaggaggatccagagatgagagcaggtcactgggaaaggttaggggcggggaggccttgattggtgttggtttggtcgttgttgattttggttttatgcaagaaaaagaaaacaaccagaaacattggagaaagctaaggctaccaccacctacccggtcagtcactcctctgtagctttctctttcttggagaaaggaaaagacccaaggggttggcagcaatatgtgaaaaaattcagaatttatgttgtctaattacaaaaagcaacttctagaatctttaaaaataaaggacgttgtcattagttctttggtttgtattattctaaaaccttccaaatcttaaatttactttattttaaaatgataaaatgaagttgtcattttataaaccttttaaaaagatatatatatatgtttttctaatgtgttaaagttcattggaacagaaagaaatggatttatctgctcttcgcgttgaagaagtacaaaatgtcattaatgctatgcagaaaatcttagagtgtcccatctggtaagtcagcacaagagtgtattaatttgggattcctatgattatctcctatgcaaatgaacagaattgaccttacatactagggaagaaaagacatgtctagtaagattaggctattgtaattgctgattttcttaactgaagaactttaaaaatatagaaaatgattccttgttctccatccactctgcctctcccactcctctccttttcaacacaaatcctgtggtccgggaaagacagggactctgtcttgattggttctgcactggggcaggaatctagtttagattaactggcattttggcttttcttccagctctaaaacaagctccatcacttgaaatggcaaaataaaatcatggatgaggccgagggcggtggcttatgcctgtaatcccagcactttgggaggccaaggtggtaggatcacgaggtcaggagatcgagaccatcctggccaacatggtgaaaccccctctccactaaaaatacaaaaattagctgggcgtagtggcatgtgcctgtaatcccagctactcaggaggctgaggcaggagaatcacttgaaccaggaggcagatgttgctgtgagccaatatggcaccactgaactccagcgacagagctaaactccatctcaaaaaaaaaaaaaaaaaaaaaaaaacatggatgatcggtgtcgttgagaggataggtatttggaagaacctttgtttgaaactggctctgtacatacaatgaaattacatacttatttacatacaatgaaatgcagaggttttttttttatataggatctctgtcgagaggctggagtgcagtggtgctatcacagctcactgcagcctcaacctcgtcaggctcaagcaatcctcccacctcagcctccagagtagcagggacgataggtgtgcaccaccatgcccagctaatttttgtatttttttttctttttttgagatggagtcttgctctgttgcccaggctggagtgcagtggcgcgatctcagctcactgcaaactctgcctcccgggttcatgccattcttctgcctgagcctcctgaatagctgggactacaagcacccactaccacgcccggctaattttttgtatttttttttcttttttagtagaggcgggatttcaccgtgttagccaggatagtcttgatctcctgaccttgtgatccacccgcctgggcctcccaaagtgctaggattacaggcataagccactgcgtccagccattcttgtatttttctgttgtagagatagggttttgctatgttggccatgctggtctcaaactcctgacctcaagtgatctaccctcccttggcctctcaaggtgctgggattacaggcctgagccattgcacccagccatggtctaaaaatcttgattgaaataccaccttttcatttccagacacccctatttaaaattaccacacccccagcacacactttatcttctattcctgctgcttctccataacactgattactagctgacattctatgtaatgtatccattttttatctctagtcccacagaatgtaaactccaggatgggatttttgttttgtttacatacatctgtatgttcagtagttagaacggtacttgggacctagttgccactcaataaacatttgtcaaataaataataaactaaactaaattagttctttaatttttttaaatatggtgatggttagtagtgagtaacattcaaaaaataagttgaaaagttgtaccattgcctcttacccacaataaaaaagggtaaattcttttctgctttatgaaagttgtttttcatatttgaagtcaagttaatcagattaaggaaaatgtatgttgtgttttcagagcgatacaagatttataaataaccatcctctcccttgcccttcaacattatagctaaacaaaaataagaggaaaacaggattcacaatttatcaatttattgaaaatcagagccagagaagcaggaaatgacattgtaggaaaaaactgcttttgaaaaagcacaaaacttactcatgacaatcagtgatcaggaaaatcctcaatagtgtggcatttggatacatttatgtttcatttccatgggagagagtcataaaaataggatgttctttctcattctggcaaattaaaccatcaattaaaaactcagatacataaaaattaaagatgtaagaatgaaaatgctaaattgttattttcaatcaactattatgttttctagcttttcattgcttttttctgtttcctgttaagattaatttctttttttttttttttttttttttttgagacagactttggctcttgttgcccaggctggagtgcagtggcacaatctcggctcactacaacctccacctcccgggttcaagcaattctgctgcctcagcctccggagtacctgggattgcaggcatgtgccatcacaccagctaattttgtatttttagtagagacagggtttctccatattggtcaggttggtctcgaactcctgacctcaggtgatcctcctgccttggcctccgaaagtgctgggattacaggcgtgagccaccgctcccagactttttgttttgttttgttttgtttttttgagacacggtctcgctctgctgcctaggctggagtgcagtggcacgatcttggctcactgccagctccgactcccgggttcaggccattctcctgcctcagcctcccgagtagctgggactacaggcgcccaccactatgcccggctaattttttgtatttttagtagagacggggtttcaccatgttagccaagatggtctcgatctcctgaccttgtgatccacccgcctcagccttccaaagtgctgggattacagtcctgagccactgcgcccggcctggacctttttttttcggggtggggggttggagtctggctctgtcgcccaggctggagtgcagtggcgccatcttggctcactgcaacctccgcctgccaggttcaagttcaagcgcttctcctgcctcagcctcctgagtagctgggattataggcgcacgccaccgtggccggctaattttgtatttttagtagagatagggtttcatcacgttggtcaggctggtcttgaagtcctgatctcgtgatccacccgcctcggccttccaaagtgctggcgtgagccactgcgcctggcttaagattaatttttgtttgttttgtttttgagacggagtctcgctctttcacccaggccggagtgcagtggcgccatctcggctcactgcaagctccgcctcccgggttcacgccattctcctgcctcggccccccaagtagctgggactacaggcgtccaccaccacgcccggctaattttttgtatttttagtagagacggggtttcaccgtgttagccaggatggtctccacttcctgacctcgtgatccgcccacctcggcctcccaaagtgctgggattacaggcgtgagccaccgcgcccggccttaagattaatttttatggtgttttacattcatttgtatggaaagttctaggatagggatcatatttcacttccttttaatatagtacagtatagcacaatttgcagttatgtcttaatatgtgatcaggaatgatcatgactggaaacagtgttatttgtggtagctatagggtaggtaaggttttcagcctgttttaggtttcttgaactaaaattccttctgctgtcttctaagtcaatattggcagctatttctgacaattggtagttctttgtaactttttacctatgactataacatttttgactttcagaagaatttgctaaaatgtgttccccggtgggttgttgtttttcaacctaaacctagctgctttttccagtcacttatccgtattggaagctcaaaatgcaaatatacagtaggcctaaaatattgcctggtttgaaaagtgtttaaaatatttgaatcatttttatagtaaacatttactctcatcaggacctagaaggggaacattttaattttttttcttttcccttttcacagtcttccttcaacattcattacctttttacatatcggagttttcatctgttcaaagtttgtgtttacagtgtgtttatatagtttagattataattaccatactgaaatataattgtttcagaattgagtcagtggtgagaatgaaagccatctggtatgataactgaatccaatttttcttttacggagaatttctttgaaatgtagcttatctcagaaatagggatttagtaaccaatcagagttttctttgtcaaggttgtttttctttttaaagtcacatttggtcccagtaataataccaatgttggtacaagttatctcaggttgtgaagcatttttcccaagtcatctcaggttgtgaagcattttcccaagtagcatttaattttattcttgcaatagcccaaggagtctggcagggtgaatggcaagagaaggaaacaggttcaggtagagtggttagcccaaggtggctctgcttatatacacaactggtagtagaaacccagcctcctgacttagttcattgtttttcttttcactgccctgtgctatgtcaaaaaccccatgattacaagagttgtattacaacccttcacaataaggttactgtccacaagcttttcttgtgatccttttcttttttttttttctttttttgagatggattctctgtcacccaggctggcccgccttggcctcccaaaatgctgggattacagcgtgagccaccgcacctggcccttgtgatccttttctaaaaagttaaatatttaaggaaaaaaccacattcttgtcacactgccaggttagtcgttctttgatatcttgcctggactttatccaaaaaatccgtttcaaaaattcacatttagagctaagtgtagtggctcacgcctgtaatcccggtcgaggcagatggatcacttgaggtcaggacttcaagaccagcctgggcaatatggtgaaaccccttccctaccaaaaatacaaaaaaattagccgggtgtggcagcacgcgcctgtagtcccagctacttggaatgctgaggcacaagaatcacttcaatccgagaggcagaggttgcagtgagccaagaccacaccactgcactccagcctgagcagcagagtgagtgagactccatctccaaaaaaaaaaaaaaaggttcacattcagaagaaagctaaaggccgggtatagtagctcacacctgtaatcccagcactttgggaagccgaagcaggaagattgcttgatgccaggcattcaagaccagcatgggcatcatagtgagatcctgtctctacaaaaattaattaacattaaaaattaaaaagatggctggcatggtggctcactcctgtaatcccagtactttgggaggccaaggcatggtggtgcatgcctttagtcccagctactcgggaggctgaggcaggagaatcacttgaattcaggaggcggaggttacagagagccgagatggtgccactgcactccagcctgggcgacagaacgagactctgtctgaaaaaaaaaaagaaaattaaaaagaccagaataaagctaaagatttaaaatagcctataggttcctaccagaagttaccagctacctctctgatagtctttccctacaatatcctcctggattattacattttagcaccttgacctatctgatgtcctgcatacacaggcatggtcctgctcagggtttgccttctctgctccctctttcttggaatgctcttcccctaattgttgcatagtgtgtttctttacattattaagctatcctctagtctcacctcagtgaaacctttcctgactccccccatgtacatctcacccccacatagatattgaactacctgtttccccttaccctgcttaatttttctctttaatgcacttattcccatgtattctttaattccgtatcaactgtctaccacactagaatatgagctctatgagagcaggctttattttgtaaactgctacatttctatctcctagaatagtacttgaatatagtagtagatacttaataaacacttgttatattagtataataaatgaactaatctcaggaatgccttggttttgtggatagacaggtagggatgggaacttgggtgatgtattttctgaagtttttatttttaagcttattattattttgagatggagtccagctctgtcgcccaggttggagtacagtggcgcgatcttggctcactgcaacgtgcacttccccggttcaagcgattctcctgccttagcctcccaagtagctgggattacaggcgcatgccaccatgcccagttagttttggtatttttagtagagacagcgtttcactgtgttggccaggctggtctcgaaatcctgacctcatgatccgcccgcctcggcctcccaagtgctgggattacaagcatgagcccccgtgtctggccttattttcttttttttgagacagagtcttcctctgtcacctaggctggagtgcagtggcacgatattggctcactctgcaacctccacctccaggattcaagtgatccttctaccttagtctccaaagtagctgagaccacaggcatgcgccaccacgcccggctaatttttgtatttttagcgtagacagggtttcaccatattgtccaggatgatctggaactcctgagctcaggtgatccacccacctcagcctcccaaagtgctaggattacaggcatgaggcaccatgcccggccttaagcttatcattttctaaatttcctttagtgagtacttattacactgtttttacaaagtaatcacaaaccaaacatcatgcctcttctgaagtgatctaataagagtacacagtaccatctgtaaagtgttcttgccagaaagttgaacctgaatgattaagcctgtaagtctagtttataggaaataaggctagaggaacaagttaaacctcaccatagggttatacaatcagcaaaatccagaatgggggaaactccacaggtcaaatgacctaattttaaaaataaatgacaagggagaaaaagtaagagacacctatagatcagaagacacttggggctgggcatggtggctcacacctgtaatcccagcactttgggaggccaaggcaggcggatcacctgaggtcaggagttcaagaccagccggccaacatggtgaaaccccaactctactaaaaatacgaaaaatcagccgggcgtggtggcgcacgcctgtagttccaactacctgggaggctgaggcaggagaatcacttgaacttgggaggcagaggttgcagtgagccgagatcgcaccattgcatgccagtctgggctacaaaagcaaaaccccatctcaaaaaaaagaagacacttgggtttgggtgtgttggctcatgcctgtaaaccccgtgctgggaggattgcttgagcccaggagttcaaggctgcagtgaggtatgtttgcaccactgcactccagcctaggtgacagagtgtgaccttatcttaaaagtaataataattaaaataatctggggtaggggtggatatgggtgaaacagcttggccatgagttgatggttgttggaccagggtgatggtccatatagttcattttattattttatttacttgaaattttgaaatacttgaaattttccatattaagttaaaaaggcatttacagtaaacaaaaaaaaagttctaggaaggaattcaaaagaaatataagcagaaaattttgtctttatggagcttaaagatgagatgtgcacccacagtgatagtgcagaaaaatatatcactggaaatgaattcgtacgaactattatcaactaatcttttaaatgctgatgatagtatagagtattgaagggatcaatataattctgttttgatatctgaaagctcactgaaggtaaggatcgtattctctgctgtattctcagttcctgacacagcagacatttaataaatattgaacgaacttgaggccttatgttgactcagtcataacagctcaaagttgaacttattcactaagaatagctttatttttaaataaattattgagcctcatttattttctttttctccccccctaccctgctagtctggagttgatcaaggaacctgtctccacaaagtgtgaccacatattttgcaagtaagtttgaatgtgttatgtggctccattattagcttttgtttttgtccttcataacccaggaaacacctaactttatagaagctttactttcttcaattaagtgagaacgaaaaatccaactccatttcattctttctcagagagtatatagttatcaaaagttggttgtaatcatagttcctggtaaagttttgacatatattatctttttttttttttttgagacaaagtctcgctctgtcgcccaggctggagtgcagtggcatgatcttggctcactgcaacctccgccccccgagttcaagcgattcttctacctcagcctcccaggtagctgggactacaggcacccgccaccatgcttggctaatttttgtacttttagtagagataaggtttcaccatattggccaggctggtctcgaactcctgaccttgtgatccacctgcctcggcctcccaaagttctgggattacaggcgtgagccaccacacccgactgacatatattatctattaggatgtaacatcattttgaacagtgttttgtattttttgtgtccatcagtgaaagcaaactgcaagcagttttgaaataagcacattgtgtttgagccttcccagtttctcctttctgttcatttctgcatatccttatgcattcccccttctaagggtcagtgtttgcccgctttgtaatcattgtgaagacaggaaaggacctgataccagtttctatttaggccaaaattcatttatagcagtgattcaagttatatttacgtatttgatgatcttgtcttttgaaatgaaaatgtttgtttcttaataaaagaatttcagaaaaagtagagtaggtaatttagtagaacaagtgggctttctccttttctttatgttaagctatggctcacatcttaccttaaatgtcaactaatttgtttttaagtatttatgtacctggtacataacctggtaccaggtacaaactatgtacttggtaaaaagtttattagcacaaaaaggtatatgatgcaaagtatacttccctcttaccctacaacccctgcctccctgttccctccccagacaaccacaatgatcaatttcttatgtatcctttgaggaatttttaaattccagagttcttaacttggggtttatgaatagtctttatgaatttcctagaattatatttaaattgtattcaaaactatggccatgtacatttttctgggaagatagtccataattttcatctgagtgagctaagatcatgccactgcattccagcctgggcgacaagagggagactcaaaaaaaaaaaaaaaaagtcccagtatttactacagagagctaaagattaacctttaaagccctggggctttcaatttatctggatgagaatctttctggaatgaactgtatgttttcttgtcagcttgagtaacaaatgctgaacatactatactattattacagggactcaagggcccagtgtggtagctcctgcccataaccccagcactttgggaggccaaggcaggaagatcacttaaggccaggagttcgaagctgtagtgagctatgatcacaccactgcactccagcctagatgacagagtgagaccctgtctttttttttttttgagatggtgtttcactctattgcccaggctggagtgcagtggtgtgatctcggctcactgcaacctccacctcctgggttcaagcgattctcctgcctcagcctcttgagtagctgggattacaggcatctgccaccacacccagctaatttttgtatttttagtcgagacagggttttcaccatgttggccaggctgctctcaaactcctgacttcagctaccttggccttaaaaagtgttgggattacaggtgtaagccaccgcgcctggctgaccctgtctcttaacaaaaaaagagagattaagttatgaatatagttgctttgagaacttgtggaagaaggaaattataggcttataggcagagataataatacgagcaaatgtacaaataaaagaaaatagaggacgggcgcggtggctcacgcctataataccagcactttgggaggtcgaggtgggcggatcacgaggtcaggaaattaagaccatcctggccaaaatggcgaaacactgtctctactaaaacacacaaaaaactagcctggcatggtggcacgtacctgtagtcccagctacttggtaggctgcggcaggggtatcacttgaacctgggaggcagaggttgccctgagccgagatcatgccaatgcactccagcctgacaacagagtgagactctgtctgaaaaaaaagaaaagaaaagaaaatacatccaggaaaaataagctaactttgcatatgtgtataggagttgtgttagaaaaggaagaagccctcaaagatgggaagccatttgcaagaaagagaaggtccaagaggaggcagaagggattggaaatagaaaaaggatgtaagaaagagttgattattactcataaacagtaatgaaggaaaaggagagtaattctacaggaagatgctgaggtgctttgagcccagtgaagttggaggtaaagacagctgttgaggccgggcacggtggctcacgcctctaatcctagcacttttggagcccaaggcaggtggatcacctgaggtcaggagctcaagaccagcctgaccaacatagagaaaccccatctctactaaaaatacaaaattagacgggcgtggaggcgcatgcctgtaatcccagttacttgggaggctgaggcaggagaatcacttgaacctgggaggcggaggttgcagtgagccgagattgcgccattgcactccagcctgggcgacaagagtgaaaactgtctcaaaaaaaaaaaacaacaaaaaacagctgttgagattgagaggattagagttggcaactggagaagagtgagaagcttggtttcaagcttgtgatagtcaggattgtgatagtcaggaaagaaccagtcataaagatatatgtgtgtgtatacatataaatatgttatatatatgtgtgtgtgtgacacatatatatttttgtttgtttctttgagacagtgtctccctctgacacccaggctggagttcagtggtgtgatcatagttcacttttaccttgcaatctgggttcaagcaatctctcatctcagcccctcaagtagctaggactacaggtacatggcatttgcccagctaatttttaagtttcttgtagagatgggccagccatattttaaattgtgttttgaatgttatattagaattaaaagtccaaagccgggtgtggtggctcacgcctgtaatcccagcactttgggaggctgaggtgggcggatcacgaggtcaggagttcgagaccagcctggccaatatggtaacaccatctctactaaaaatacaaaaattagctgggtatgggggcacatgcctgtagtcccagctactcaggaggctgaggcagaggaacctcttgaacccaggaggcagaggctgcagtgagttgagatcgtgccactgtactctagcctgggcgacagagcaagattccgtctcaaaaaaaaaaaaagtccagtataatgcccatgtgatagatcgactttttcatgaaatctcttctgtaatatcaatataatctgaataacactttgatctatatgatgagaaagctgggagcctgggagcgatacccccatgcttttgttgtattaattgtattttctacggataaactctaattgctaaaaataaaacaactttattgacccaagcaagcctaaagttctgaaatcttttttttatttttgtttgtttgtttgtttgtttttgtttgttttgttttgagacggagtctcgctctgtcgcccaggctggagtgcggtggtgcagtctcggctcactgcaagctccacctcccgggttcacaccattctcctgcctcagcctcccaagtagctgggactacagacgcctgccaccacgcccagctaatttttttgtatttttagtagagaaagggtttcaccgtgttagccaggatggtctcgatctcctgacctcgtgatctgcccgccttggcctccctaagttctgggattacaagtgtgagccaccacgcccggctgtttttttttgttttgttttgagacggagtctcactgtgttgcccagactggagtgcagtggcatgatctcagctcactgccacctccatctcctgggttcaagcaaatctcctgcctcagcctcccgagtagctgggactacaggcatgtgccaccacacctggctaatttttgtatttttagtagagacggggtttcactatgttggccaggctggtccaaaactcctgacctcaggtgatctgctcgccttggcctcccacagtgccaggattacaggcatgagccaccttgcccagccagttctgaaatcttttatgaagcctataaaaaaagataataataccaatctagaaaatatttcttaaggcagtcatgcattagtttgaactttccaaacaaaaaaatgcaatgtgtaatacttttttttttttttttgagatggagtcttgttctgttgcccaggctggagtgcagtggtacaatctcggctcactgcagcctctgcctctctggttcaagtgattctcctgcctcagcctcccaagtagctgggattacaggcgtgcaccaccatgcatggctaatttttgtatttttagtagagacagggtttcaccatgttgacaaggctgatctcgaactcctgacctcaggtgatccgcccacctcagcctcccaaagtgctgagattacaggcattagccaccacgcccagccttttattttagtagagaccatgtttcaccatgttgaccaagctggtcttgagctgacctcaagtgatccgcccacctccacctcccaaaatggtgggattataggcatgagccaccgcacccagcctgtaatacttttttgaagatctagaaccacattgttcaaagagatagaatgtgagcaataaatgtaacttaaatttttcaacagctactttttttttttttttttgagacagggtcttactctgttgtcccagctggagtacagtggtgcgatcatgaggcttactgttgccttgacctcctaggctcaagcgatcctatcacctcagtctcccaagtagctgggactgtaagtgcacaccaccatatccagctaaattttgtgttttctgtagagacggggtttcgccatgtttcccaggctggtcttgaactttgggcttaacccgtctgcccacctaggcatcccaaagtgctaggattacaggtgtgagtcatcatgcctggccagtattttagttagctctgtcttttcaagtcatatacaagttcattttcttttaagtttagttaacaacctttatacatgtattctttttctagcataaagaaagattcgaggccgggtgcggtggctcacgcctgtaatcccagcactttgggaggctgagatgggcacatcacgaggtcaggagatcgagaccatcctggctaacatggtgaaaccccgcctctactaaaattacaaaaagttagccaggcgtggtagcgggcacctgtagtcccagctactcaggaggctgaggcaggagaatggcgtgaacccaggaggcagagcttgcagtgagcagagattgtgccactgcactccagcctgagagacagagcgagactccgtctaaaaaaaaaaaaaaagattcgaatccttatcttggttgatttttgcgtatctagttccactgaattatttatataattgtatagactacagcacgagacagcttagcttgtcactctactgtactatattctgcagtactatcataagggaatttcctccctacccctgctctgaattgttcaattgtactatttgctggagtaatgcttgatgccttcttgatccattatactagagtatatgtagtatttgtagattctgaaggagtgggagcctctattctgagttttaaaggtacttatgtacagtggaggtagctttttgacagcctcatcttccaaactatagagtcattgttttgttgagtgcaatatggtacttgaagcatctatatcggcgaagaaggacccaagtctccttgaccttacctacctacattcactttctctggtaggaagattgtgggtgcctctctccagacttagtttccatgtcaaaaaagaaaaaaggaagattgtgggctttgctacaatccaattctggatccaatataaccttcattgcttaattactgtgtgatctgggacaagcctctactctataaaaatgaagataaggccaggcttgatggctcatgcctgtaatcccagcacgttgggatgccaaggcaggaggatcacttgaggtcaggagttcgagaccagactgggcaatatagtgaaaccacatctgtacaaaaataaagatagaaagtagcccagcgcaatggctcacacctgtaatcccagcactttgggaggctgaagcaggcgatcacttgaggtcgggagttcaagactgtagacagatagataggtaggtagatagataggtaggtagatagatagagatatagatatagttggggtttttttgttttgttttgttttgtttttgagatggagtttcgctcttgttgcccaggctggagtgcaatggcgcgatctcagtttactgcaacctccgcctcccgggttcaagagattctcctgcctcagcctcctgagtagccaggattacaggcatatgccaccatgcccggctaatttttgtatttttagtagagacagggtttctccgtgttggtcaggctggtcttgaactcctgacctctcccaaagtgttgggattacaggcgtgagccaccgctcctggccttttttttttttttttttttttttttgagacagagtcttcctctgttgcccagggtggagtgcagtggcactcttctcagctcattgcaacctctgccatcctgggttccagtgattctcatgcctcagcctcccaagtagctgggactcaggcgtgtgcccaccacgcctggctaattttgttgtatttttagtagagacagggtttcaccatgttagccaggctggtctcaaactccaggcctcaagtgatctgcctgcctcagcctcctgggattgcagacatgagccactgcacccggccaagagagggtaataaatgttaaattacctggctagtaaaaaatattctctaagtgtcttttctcacaattcccaatgccttttttttttttttggcacaatctcactctgttgcccaggctggaatgcaatggtgcaatattggctcactgtaacccccgcctcacaggttcaacttattctcatgcctcagcctcccgagtaactgggactacagtgcaccaccaccacacccagctaatttttgaatatttagtagagacagggtttcaccatgttggccaggctggtcttgaactcctggcctcaagtgattcacccaccccgcaagtgctgggattacaggtgtggaccaccgtgcacagccctagtgactttttttttagccccttaatcttttctttcctgggtctcttcattgtcagtgtctgctatttactccctacctagtcacccccttcaccagtatattatgtcctttatgttttattttgcaggatcttattttgcttttctattgaatcccctccatctagaatagtactagacatagtaaatattggttgtatgagtgaatcgctgcttttaattatcatcaccattgctctctctacttctggtctatgatccactttgagttaacttttgttatttggtgtgagataggagtataatttcattcttttacatgtggttatacttttgtctcaacactgtttgttaaaaacacaaaaagtattattttcccatttaatcatctttggcctgggcacggtggctcatgcctgtaatcccagcactctggaaggccaaggcagatggatcaatttgaggccaggagttcaagactagccaacatggtgaaactaaaaatacaaaaaattagctgggtatggtggtgcatgtctgtaatcccagctactcgggaggctgaggcacgagaattgcttgagcctaggaggtggaggttgtagtgagctgagattgtgtcactaccctccagcctgggtgatagagtgagtctgtctcaaaaaaaaaaaaaaaaaattaagaaaataaaaatcgtcggccaggcatggtggctcacacctgtaatcccagcactttgggaggcagaggcgggcagatcacgaggtcaggagatggagaccatcctggctaacatggtgaaaccccgtctctactaaaaataaaaaaattagccgggcatggtgctgggcgcctgtagtcccagctgctcgggaggctgaggcaggagaatggcgtgaacccaggaggtggagcttgcagtgagccgagatcgtgccactgcactccagcctgggagacagagcgagactccgtctcaaaaaaaaaaaaaaaaaaaattgtcttggtatttattattgttgaaaatcgcttgatcacagatgtatgtatgagtttatttctgtactgtcaattccattttattgatgtatgtgtctattcttatgctattaccacactttcttgattactatagctttgtggtgaggtgttgagattttaaactaattataagcatcttacatgaactacttaccgtttatatttgattatgcagcatgaaataattatgaatatatcattaaatatgccatattaacttttattaagttttatgtgatcataacagtaagccatatgcatgtaagttcagttttcatagatcattgcttatgtagtttaggtttttgcttatgcagcatccaaaaacaattaggaaactattgcttgtaattcacctgccattactttttaaatggctcttaagggcagttgtgagattatcttttcatggctatttgccttttgagtattctttctacaaaaggaagtaaattaaattgttctttctttctttataatttatagattttgcatgctgaaacttctcaaccagaagaaagggccttcacagtgtcctttatgtaagaatgatataaccaaaaggtatataatttggtaatgatgctaggttggaagcaaccacagtaggaaaaagtagaaattatttaataacatagcgttcctataaaaccattcatcagaaaaatttataaaagagtttttagcacacagtaaattatttccaaagttattttcctgaaagttttatgggacatctgccttatacaggtattagaaacttactgcctttctctaatgcttctagtgtaaaaacttgcagacttatgtaaagtagggctgtatcgccgtgcccccattgtctgttaatcttgtttttatatttttgattgtgtttccttttctttttttttttttttttaagacagggtcctgctctgtcactgaggctggagtgcagtggcgtgatctcggctcactgtagcctctgtctcccagcctcttcctgccttagcctcccaaatagctgggactacaggcacacgctaccatgcccggccaatttttgtattttttgtagagatgaggttttaccatgttgcccaggctggtaactcctgagctcaggtgatctgcccacctcggcctcccaaagtgctggggttcacaggtgtgtgtttatttctatctaattatttacacaaacacaatgtatttatatattgtgtatctcttctgctacaatgtaaattctatgagagtagtaattttgtctgtctcaacactgtttttcctaagtttggtacatagtaggcactcagatgcttaaaggaatgaatgaattgtgctttaattccactttactaaacccaaatctccctttggacattgttatctatgtgttttcaaagaagtataatcataatttgacagaaatccttgagaggcagaactaagtgagggattgggcagggttcagatgttaagaacagtaagctcagcagggtgtgattgctcatgcctataaccctagcactctaggaggctgaggtgggatgattgcttgaggccaggagtttgaaatcagcctgggcaacatagtgagaccccatcactaccaacaaaataaataaataaatgtacatggtggcatatgcccatagtcctagctacttgggaggctatagtgggaggatagcttgagtacagaagtctgaggctgcagtgagctatgattgtggcactgcatgctagcctgggcaatagagcaagaccctgtctctaaattaaacaaaaaaaaaagtactctagttttctatgcaatgcattatatctgctgtggatttagggcagtattatatcagataattttaggcatttggtaggcttaaatgaatgacaaaaagttactaaatcactgccatcacacggtttatacagatgtcaatgatgtattgattatagaggttttctactgttgctgcatcttatttttatttgtttacatgtcttttcttattttagtgtccttaaaaggttgataatcacttgctgagtgtgtttctcaaacaatttaatttcaggagcctacaagaaagtacgagatttagtcaacttgttgaagagctattgaaaatcatttgtgcttttcagcttgacacaggtttggagtgtaagtgttgaatatcccaagaatgacactcaagtgctgtccatgaaaactcaggaagtttgcacaattactttctatgacgtggtgataagaccttttagtctaggttaattttagttctgtatctgtaatctatttttaaaaaattactcccactggtctcacaccttattttatcaatcgtaaggtgcacatttttcacatcttaacatctctgaaattgggaacattttactattgagggtgtgtcatttgtttaatttgtgtgctttctttcttagtgatacagaaaataatagtgcaacttacattgttggtgtcttagctttagtgaaatacagtattgataggcaaatttcttagtgttaaggtagaaaacaaggactctaaataactttgatggtctgtgtatttgtttttgtttcctaggagtaaaatttccagttgattttttaaaatttgatttttaaaaaaaatcacaggtaaccttaatgcattgtcttaacacaacaaagagcatacatagggtttctcttggtttctttgattataattcatacatttttctctaactgcaaacataatgttttcccttgtattttacagatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttggtaaaaccatttgttttcttcttcttcttcttcttcttttcttttttttttcttttttttttttgagatggagtcttgctctgtggcccaggctagaagcagtcctcctgccttagcccccttagtagctgggattacaggcacgcgccaccatgccaggctaatttttgtatttttagtagagacggggtttcatcatgttggccaggctggtctcgaactcctaacctcaggtgatccacccacctcggctccccaaattgctgggattacaggtgtgagccactgtgcccggccggtaaaaccattttcatttattctggcaacatctctttattgagcattgtgaatatgttagtgaatgtgctagatgctcatagatttatataaaaagttagtgaagaaggaaagatggtatattaagtggttagacaagtgttctaatcagttagagttcagagaaggtcagggtacctgatataatcaagagagagaccttacagccaggtgaggtgaatgtacctataatcccagctacttaggaggctgaaatgggaggatcacttgagtccaggtttgagaccagcccaggcaacatagcaagatccccatcagatacaccaaaaagacagatttcttttttttttttttttttgagacagagtctcgctctgtcgcccaggctggagcgcagtgacacgatgtcagctcactgcaacctccgcctcccaggttcaagtgattctcctgcctcagcctcctgagtagttgggactacaggggtacgacaccagacctggctaatttttgtaattttagtagagtcggggtttcaccatattggtcaggctggtctcgaactcctgacctcaggtgatccaccctccttggcctcccagagtgctgggattacaggcgtgagccaccaagcccggccaaaaaagagagctcttataggcccttccttgctttggagctttatctgctctgtgatgcttatctaaaatagccataaggtcactgatatttttaagcatttggaaattacttcagctgggtgccatggctcatgcctataatcccaaccctttgggaggctgaggtaggaggtcctttgagcccagcttgggcaacacagtgagacactgtctctgcaattaaaaaaaaaaaaaaagtagctgggtgccgtggctcacgcctgtaattccagcactaggaggcttgaggattgcctgagctcaggagttcaagaccagtttgggcaacatagcaagtccttgtctatattaaaagtttttttaaattatctgggcatggtggtgtgtgcctgtagtcccagctacttgggaagctgagacagaaggatcacttgagtccaggagatgtagactacagtgagctatgatcactccactgcacttcagcgtgggcggcaaagcaagatctagttgcaaaaaaaaaaagaactggctgggtgcggcggctaacacctgcaatcccagcaccttgggaggctgaggccagtggatcatgaggtcaggagattgagaccaccctggccaacatggtgaaacccggtctctactaaaaatacaaaaattagctgggtgtggtggcacgtgcctgtaatcccagctactccagaggctgaggatggagaatcacttgaacctgagagtcggaggttgcagtgagccgagattgcgccactgcactccagcctggcgacagagcgagactccgtctcaaaaaaaaaaaaaaaaaagcttcacgcctgtaatcccagcactttgggaggccgagtcaagtggatcacgaggtgtggagatcaagactatcctggctcacatggtgaaagcccgtctctactaaaaacacagaaaaattagctgagcgtgatggcggactcctgtagtcccagctactcgggaggctgaggcaggagaatagcatgaacccgggaggtggagcttgcagtgagccgagatcccgccactgcgatccagcctgggcgacagagtgagactctgtctcaaaaaaaaaacaaaaaaacttagctgggcgtggtggtatgcacctgtggtcctagctacttgggaggctgaggctggagcattgctttaacatagagagtcaaggctgcagttgagctatgactgtgccactggactccagcgcaggtgactgagaccctatcttttaaaaaaagggaaaattacttgaacttaaaaggtgtaattgttaaagaaaatgtagtgatttgctctgttgttacttatatgtgcatgaatgatggagatcttaaaaagtaatcattctggggctgggcgtagtagcttgcacctgtaatcccagcacttcgggaggctgaggcaggcagataatttgaggtcaggagtttgagaccagcctggccaacatggtgaaacccatctctactaaaaatacaaaaattagctgggtgtggtggcacgtacctgtaatcccagctactcgggaggcggaggcacaagaattgcttgaacctaggacgcggaggttgcagcgagccaagatcgcgccactgcactccagcctgggccgtagagtgagactctgtctcaaaaaagaaaaaaaagtaattgttctagctgggcgcagtggctcttgcctgtaatcccagcactttgggaggccaaggcgggtggatctcgagtcctagagttcaagaccagcctaggcaatgtggtgaaaccccatcgctacaaaaaatacaaaaattagccaggcatggtggcgtgcgcatgtagtcccagctccttgggaggctgaggtgggaggatcacttgaacccaggagacagaggttgcagtgaaccgagatcacgccaccacgctccagcctgggcaacagaacaagactctgtctaaaaaaatacaaataaaataaaagtagttctcacagtaccagcattcatttttcaaaagatatagagctaaaaaggaaggaaaaaaaaagtaatgttgggcttttaaatactcgttcctatactaaatgttcttaggagtgctggggttttattgtcatcatttatcctttttaaaaatgttattggccaggcacggtggctcatggctgtaatcccagcactttgggaggccgaggcaggcagatcacctgaggtcaggagtgtgagaccagcctggccaacatggcgaaacctgtctctactaaaaatacaaaaattaactaggcgtggtggtgtacgcctgtagtcccagctactcgggaggctgaggcaggagaatcaactgaaccagggaggtggaggttgcagtgtgccgagatcacgccactgcactctagcctggcaacagagcaagattctgtctcaaaaaaaaaaaacatatatacacatatatcccaaagtgctgggattacatatatatatatatatatatatcatatctatatatatatatatgtaatatatatgttatatatatattacatatatatatgttatatatatgttatatatatataatatatatatgttatatatatgttatatatatatatacacacacacacacatatatatgtatatatatatacacacacacacacatattagccaggcatagttgcacacgcttgtagacccagctactcaggaggctgaggcaggagaatctcttgaacttaggaggcggaggttgcagtgagctgagattgcgccactgcactccagcctgggtgacagagcaggactctgtacaccccccaaaacaaaaaaaaaagttatcagatgtgattggaatgtatatcaagtatcagcttcaaaatatgctatattaatacttcaaaaattacacaaataatacataatcaggtttgaaaaatttaagacaacagaaaaaaaaattcaaatcacacatatcccacacattttattattactactactattattttgtagagactgggtctcactctgttgcttatgctggtcttgaactcctggcctcaagcagtcctgctccagcctcccaaagtgctgggattataggcatgagctaccgctcccagccccagacattttagtgtgtaaattcctgggcattttttccaggcatcatacatgttagctgactgatgatggtcaatttattttgtccatggtgtcaagtttctcttcaggaggaaaagcacagaactggccaacaattgcttgactgttctttaccatactgtttagcaggaaaccagtctcagtgtccaactctctaaccttggaactgtgagaactctgaggacaaagcagcggatacaacctcaaaagacgtctgtctacattgaattgggtaagggtctcaggttttttaagtatttaataataattgctggattccttatcttatagttttgccaaaaatcttggtcataatttgtatttgtggtaggcagctttgggaagtgaattttatgagccctatggtgagttataaaaaatgtaaaagacgcagttcccaccttgaagaatcttactttaaaaagggagcaaaagaggccaggcatggtggctcacacctgtaatcccagcactttgggaggccaaagtgggtggatcacctgaggtcgggagttcgagaccagcctagccaacatggagaaactctgtctgtaccaaaaaataaaaaattagccaggtgtggtggcacataactgtaatcccagctactcgggaggctgaggcaggagaatcacttgaacccgggaggtggaggttgcggtgaaccgagatcgcaccattgcactccagcctgggcaaaaatagcgaaactccatctaaaaaaaaaaaagagagcaaaagaaagaatatctggttttaaatatgtgtaaatatgttttggaaagatggagagtagcaataagaaaaaacatgatggattgctacagtatttagttccaagataaattgtactagatgaggaagccttttaagaagagctgaattgccaggcgcagtgctcacgcctgtaatcccagcactttggaaggccgaggtgggcggatcacctgaggtcgggagttcaagaccagcctgaccaacatggagaaaccccatctctactaaaaaaaaaaaaaaaaaaattagccggggtggtggcttatgcctgaaatcccagctactcaggaggctgaggcaggagaatcgcttgaacccaggaagcagaggttgcagtgagccaagatcgcaccattgcactccagcctaggcaacaagagtgaaactccatctcaaaaaaaaaaaaaaagagctgaatcttggctgggcaggatggctcgtgcctgtaatcctaacgctttggaagaccgaggcagaaggattggttgagtccacgagtttaagaccagcctggccaacataggggaaccctgtctctatttttaaaataataatacatttttggccggtgcggtggctcatgcctgtaatcccaatactttgggaggctgaggcaggtagatcacctgaggtcagagttcgagaccagcctggataacctggtgaaacccctctttactaaaaatacaaaaaaaaaaaaaaattagctgggtgtggtagcacatgcttgtaatcccagctacttgggaggctgaggcaggagaatcgcttgaaccagggaggcggaggttacaatgagccaacactacaccactgcactccagcctgggcaatagagtgagactgcatctcaaaaaaataataatttttaaaaataataaatttttttaagcttataaaaagaaaagttgaggccagcatagtagctcacatctgtaatctcagcagtggcagaggattgcttgaagccaggagtttgagaccagcctgggcaacatagcaagacctcatctctacaaaaaaatttcttttttaaattagctgggtgtggtggtgtgcatctgtagtcccagctactcaggaggcagaggtgagtggatacattgaacccaggagtttgaggctgtagtgagctatgatcatgccactgcactccaacctgggtgacagagcaagacctccaaaaaaaaaaaaaaaagagctgctgagctcagaattcaaactgggctctcaaattggattttcttttagaatatatttataattaaaaaggatagccatcttttgagctcccaggcaccaccatctatttatcataacacttactgttttccccccttatgatcataaattcctagacaacaggcattgtaaaaatagttatagtagttgatatttaggagcacttaactatattccaggcactattgtgcttttcttgtataactcattagatgcttgtcagacctctgagattgttcctattatacttattttacagatgagaaaattaaggcacagagaagttatgaaatttttccaaggtattaaacctagtaagtggctgagccatgattcaaacctaggaagttagatgtcagagcctgtgctttttttttgtttttgtttttgttttcagtagaaacgggggtctcactttgttggccaggctggtcttgaactcctaacctcaaataatccacccatctcggcctcctcaagtgctgggattacaggtgagagccactgtgcctggcgaagcccatgcctttaaccacttctctgtattacatactagcttaactagcattgtacctgccacagtagatgctcagtaaatatttctagttgaatatctgtttttcaacaagtacatttttttaacccttttaattaagaaaacttttattgatttattttttggggggaaattttttaggatctgattcttctgaagataccgttaataaggcaacttattgcaggtgagtcaaagagaacctttgtctatgaagctggtattttcctatttagttaatattaaggattgatgtttctctctttttaaaaatattttaacttttattttaggttcagggatgtatgtgcagtttgttatataggtaaacacacgacttgggatttggtgtatagatttttttcatcatccgggtactaagcataccccacagttttttgtttgctttctttctgaatttctccctcttcccaccttcctccctcaagtaggctggtgtttctccagactagaatcatggtattggaagaaaccttagagatcatctagtttagttctctcattttatagtggaggaaataccctttttgtttgttggatttagttattagcactgtccaaaggaatttaggataacagtagaactctgcacatgcttgcttctagcagattgttctctaagttcctcatatacagtaatattgacacagcagtaattgtgactgatgaaaatgttcaaggacttcattttcaactctttctttcctctgttccttatttccacatatctctcaagctttgtctgtatgttatataataaactacaagcaaccccaactatgttacctaccttccttaggaattattgcttgacccaggtttttttttttttttttttggagacggggtcttgccctgttgccaggatggagtgtagtggcgccatctcggctcactgcaatctccaactccctggttcaagcgattctcctgtctcaatctcacgagtagctgggactacaggtatacaccaccacgcccggttaattgaccattccatttctttctttctctcttttttttttttttttttgagacagagtcttgctctgttgcccaggctggagtacagaggtgtgatctcacctctccgcaacgtctgcctcccaggttgaagccatactcctgcctcagcctctctagtagctgggactacaggcgcgcgccaccacacccggctaatttttgtatttttagtagagatggggtttcaccatgttggccaggctggtcttgaactcatgacctcaagtggtccacccgcctcagcctcccaaagtgctggaattacaggcttgagccaccgtgcccagcaaccatttcatttcaactagaagtttctaaaggagagagcagctttcactaactaaataagattggtcagctttctgtaatcgaaagagctaaaatgtttgatcttggtcatttgacagttctgcatacatgtaactagtgtttcttattaggactctgtcttttccctatagtgtgggagatcaagaattgttacaaatcacccctcaaggaaccagggatgaaatcagtttggattctgcaaaaaagggtaatggcaaagtttgccaacttaacaggcactgaaaagagagtgggtagatacagtactgtaattagattattctgaagaccatttgggacctttacaacccacaaaatctcttggcagagttagagtatcattctctgtcaaatgtcgtggtatggtctgatagatttaaatggtactagactaatgtacctataataagaccttctgtaactgattgttgccctttcgtttttttttttgtttgtttgtttgtttttttttgagatggggtctcactctgttgcccaggctggagtgcagtgatgcaatcttggctcactgcaacctccacctccaaggctcaagctatcctcccacttcagcctcctgagtagctgggactacaggcgcatgccaccacacccggttaattttttgtggttttatagagatggggtttcaccatgttaccgaggctggtctcaaactcctggactcaagcagtctgcccacttcagcctcccaaagtgctgcagttacaggcttgagccactgtgcctggcctgccctttacttttaattggtgtatttgtgtttcatcttttacctactggtttttaaatatagggagtggtaagtctgtagatagaacagagtattaagtagacttaatggccagtaatctttagagtacatcagaaccagttttctgatggccaatctgcttttaattcactcttagacgttagagaaataggaggtgtggtttctgcatagggaaaattctgaaattaaaaatttaatggatcctaagtggaaataatctaggtaaataggaattaaatgaaagagtatgagctacatcttcagtatacttggtagtttatgaggttagtttctctaatatagccagttggttgatttccacctccaaggtgtatgaagtatgtatttttttaatgacaattcagtttttgagtaccttgttattttttgtatattttcagctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtattggaaccaggtttttgtgtttgccccagtctatttatagaagtgagctaaatgtttatgcttttggggagcacattttacaaatttccaagtatagttaaaggaactgcttcttaaacttgaaacatgttcctcctaaggtgcttttcatagaaaaaagtccttcacacagctaggacgtcatctttgactgaatgagctttaacatcctaattactggtggacttacttctggtttcattttataaaagcaaatccaggtgtcccaaagcaaggaatttaatcattttgtgtgacatgaaagtaaatccagtcctgccaatgagaagaaaaagacacagcaagttgcagcgtttatagtctgcttttacatctgaacctctgtttttgttatttaaggtgaagcagcatctgggtgtgagagtgaaacaagcgtctctgaagactgctcagggctatcctctcagagtgacattttaaccactcaggtaaaaagcgtgtgtgtgtgtgcacatgcgtgtgtgtggtgtcctttgcattcagtagtatgtatcccacattcttaggtttgctgacatcatctctttgaattaatggcacaattgtttgtggttcattgtctccttaaattagactgtaagcaccttgatggaactcatactaccttttatttcacacacacgcacacgcgcacgcagcgacacagcctacacatacactgcctagctcattgtagcatactaaatactgattttaatgaataagctaaaccttcgaaacccatttgctaatcccagcactttgggaggccaaggtgggtggatcacctcaggtcagaagtttgagaccagcctggccaacatggtgaaaccccacatctactaaaaatacaaaaattagctgggcgtggtggccaatgccttgtaatcccagctattctggaggctgagacaggagaatcgcctgaacctgggaggcggaggttgcactgagctgggattgtaccactgcactccagcctgggtgacaaagtgagactccatctcaaaaacaaacaaacaaaaacacatcatttcccctatagcaaaaacatgacggcacttactgtatcaagagaggtgagaaaaaggagccacagcaggatgattcaagggactctgcatagctccattttaagaatatgcctactgcaggtcagagaaggtaagcaaactgcctaaggccacacagccaggtacagaactctcaccaatattattgccagcaatcgcaattttggtgtttattcttggtaccaagttggagactatagggttctcttcctaatagagaccatctagcctttcactgttttgtggatacttctttctcttcttctttttttttttcccttttaaaatctagttatttttttctttttggtttctttgacacagggtctcttactctgttacccaggctggaatggagtagtgcagtcatggttcactgtagctttgacttcctgggctcaagcgatcctcctacctcagcttcccgagtagctgggaccacaggcgcccaccaacacctccagctaatttttaagtttttactagagacaacatctcactatgttgcccaggctggtctcaaaatcctgggctcaagtgatcccacctcagcctcccaaaatgctgggattacaggtgtgtgcaaccacgcctggcctattttttttttaattgctcataaatcatcttttttctttaaaaaaaagaaagatgggaggctaaagcaggagaatcacttgaacccaggaggcggaggttgcagtgagctgagatcatgctgctgctctccagcctgggcaacaagagtgaaactccatctcaaaaaaaaaaaaaaagaaagtacacaattttactttctggacctaatggtcaaggccaataatttggtcacctatgaaataaataaaagctttaccatatatatgaccatttgataatgtaatatgaaatgtttatgtactaaaggcagaatagtctagaaaaaacattctgtatcacaacgtctaaaaatgaatatcatcttcatcatagaaccaggctctttctcctaatttttttttttgagatggagttttgctctgtcacccaggctggaatgcagtggcacaattttggctcactgcaaccttcagctcccaggttcaggatcaagtgattttcgtgcttcagccttctaagtagctgggattacaggtgactgccaccacacccagctcatttttttgtatttttttagtagagagagggtttcaccatgttggccaggctcgtctcgaactcctgacctcaaataatccacccgtctcagcctcccaaagtgctgagattacaggcgtgagccaccaggcctggcctcctaatttttatttgtagaagtggcaccaaaattttccaagttctcatgcaaaaattcaggctcatctcagtttatttttttcatttatttatctcccactaaattgacaacttctaataattaggttggttctttgtattcccagcacagggttctatgcagaatacacacacagcagttgctggcaataatattggtgagagttctgtactgggctatgtgatcttagacagtttgcttatgttctctgacctgccgtaggcacattcttaaaatgaagctgttcagaccccctcgattcatcctgctgtggcttctttttcccacctaaatcttaaatacccttttagctgctagtaagtgaatgatgtttttttatgaactttctgaagtcagattagatgaagttgagaaaagcctgatattcttataaagttatatatgtgcatcatagaaaacttagaaaatacagataaacaaaaatcatccatggacgaaccttgaagacattgtgttaactgaaataaaccggacaccaaaggacacatgttatatgcttccacttatatgagatacctagaatagttacatttggttactctgggtacattgcctatagataagccttgctccacaaggagcagttaaaaaaaaaaaaaagataaattcataggatggaaggtagaatagtggttactagggacttggggagggggaaatggggagttactgtttgatgagtgcagatttcagtttgggatgatgaaaaagttctggagatagatagtggcaatggtaacacaacagtgtgaaaataatgccactgaactgtacacttaaaatgattaaaatgataagttaattgtaatttgtgttatccagaaatggttagcaatttattggtgtatattcttttagtattcctgtgtgtgcacaggggtgcttgtatatactttatctttaaaatatatccaggaagctaggcacagtggcttacacctgtaatcccagcactttgggagggtgaggcaggaagattgcctgagccccggaggtcaaggctgcagtgagttgtgatcacgctactgcactctgttctgggcaacccctgtctgggaaaaaaaaaaaaattagtgaggcttagtggtgcacacctgtagtctcagctacttgagtggctggggtaggattgcttgatcccagcaagttgaggccgtggtgagccatgatggtgccactgcactccatcctgggtgatatggtgagaccctgtctcaaaaacaagaaatccagataattctgtgcattataatctagcttttactggatcattaaaattcttttttctttttttttttttttttctgagatggagtttcactcttgttgcccaggctggagtgcagtggtgtgaccttggctcaccgcatcctctgcctcccgggttcatgcgattctcctgcctcagcctcccgagtagctgggattacaggcatgtgccaccatgcccagctaactttgtatttttagtagagacagggtttctccatgttgaccaggctggtctcaaactcctggcctcaagtgatccacccacctcggcctcccaaagtgctggggttacaggcgtgagccaccgcactcagcctgggtcgttaaaattcttaagtgacttcatttttaattactatatgggattctatctttccagtgtatcatgatttatttgacctattgctgaatgttggaggtttcagggtaagaggcacagtttgctattatgtacatcactatagtggcatcctgatagctaaatatttgcctacatccctgattatttccttagtctaaattactgggactaggattttggtgtttgatacatgttactaaattgttttttagaaagattaaaccagtttatgctcttccagcccctgtggtatatgatagttcccattttcctgtaccttgccaacactgggtgatatccagttttaaaatctaaatcttgcattgctatgagaactacaattagagaaggcttatcttctactgcccattctctgtacagagcaaatccctctagacctgaagccccttggagttgtcaagaaacctttgagatgactccccactctgtatctgagctgtcaccagtattctccacttcttcaggattgccatggcaactaaattgatgaaaagatttaggaggccttttctctctttgcaattcctatgatcctttttgaatgtgggtttgggactctgtcaatatacccatcatctaattctgtccattgtgttttaaagtttaaggttgcaatttctgattacatctgccttagccatactgtattatatttgacattcaatatacaatgtccttgtttttctgtatttctaatcttattcccagagatgtgtctatttgttcaggattcattttgcaacgtgtttttactaagcatctacccaaaaccgttgaagtcagatttcaggctgtcttacgtctaaagtagcacaggcaggaaaaactattgaagtgggatttttttttccctttttgtactgaaccgagaaaaagtatatagatgatagagaattcctaatttggtatcattgatatctgggtttttgtttgtttttacagaagactgattaactatacttatttattaatttatcttctcattaataaacacttgctgagtgcttactgtctgctaggcattagggagacaaatatgattaagggaagcttcctcctatcaaggtcatgtgttccatttgggtatactaatgcattagcaatgtaaatcaagtagtgagagatcatctgttcccgataggagatggattattggtggggacttctgtgtgtgtgtgtgtgtgtgtgtgtgtgtgtgtgtgtgtatgtatgtgataaaataaatataggaaatgttaattatagattctaagtagtagatatataaacactcattgcaaagttgcttcaagttttctgtatatttgaaaatattcacaacatgtcgacaaaactagcatgataaagccactatttgtgctaagacttcagcttgtatctggattaggcttattatgtagtagtaggaacattagaaatagttttaactcattaaatacacatgttttatgggaaggttttatatatatatttatatgtaatgaatgtgaacaaacaagggtcagatatacactctgcttccctccagaccagttccggctgctctgctgcacatttcaggagtcttattagaattagccacattctgcccacttgcccttacttctcatatttcacaactcctcctggtggggactttaaggagacattcaaactaggccttgaaagatgagaatttttccaagtggaaaaagaggagtggcagcaagtaaggtaaaggtacagagtcatggaattcccaggaaacgtaaagttgtcatgtgttataggaaaacaacttgtgtgaggggtgttgggagaaatgagagataataccagggtataaagggccttttgaatgctatgttgaggaattttatcctaatggcagtaatgactaacaattatatagtgttcaaaaagtataaatcagcagtggtataccactaagggtttttttcttttctttttttttttgagacagagttttgctctgttgcccaggctggagtgccgtggcacgatctcagctcactgcacttccgccacctgggttcaagtgattcttctgcctcagccagtgtttcactgtgatggccaggatggagcactaagggtctttatggaagaaaaagacatgataaacaaggcttttagggaacttctacagtaatgtagctgtattaaaagtagagatcagagcagcatagtagaagtagaaggctagagctaattgaaggagcacttcagaattagaatcaagaagtcttagaaacctattggttttattctccctaatgtatttggccacttacctgctggggaatttgtctaagttataaaaaataattcctttgggaaacccaaaggaaagttatctattaataattaccccactactttttctgatttatgtaatggccacgtagaggttagatgtgatggttgtgacagtagtgactaatacagcctgtgaagcattttggtcagatatctatgtgctttcattccaggttgactgaggcaagactttggctagggtttgatcagtgatgtaactactcacgagtaccacgtggtggcaatggcattgctgcagaccttggcagcaaagcagtgttagagtagcagtagaaacctttgtgaagctaggaatacattttctggtcataaaaacctcctgaaaattgtgaactcagtgtagcaggagaaagaagatggcttgtttttagtaaagggcaaagtcatttttaaggatcagaagaagaaacggagagtgaaacaatgtgttcctgccctactcccccactggactttttggcaaccattgctgttccttctaaaagtgatttttaaacatgtatattttgaagccaggcacagtgactcacgtctgtaatcccagcactttgggaggccgaggcgggcagatcacctgaggtcaggagttcaagaccagcctggccaacatggtgaaaccccgtctctactaaaaatacaaaaattaggccaggtgtggtggctcacgcctgtaatcccagcactttgggaggccgaggcgggaggatcatgtggtcaggagatccagaccatcctggctaacacggtgaaacaccatttctactaaaaatacaaaaaattagctgggcatggtggcgggcgcctgtaatcccagctactcaggaggctgaagcagaagaatggcttgaacctgggaggcggagcttgcagtgaaccaagattgcgccactgcactccagcctgggcaacaaagtgagactccgtctcaaaaaaaaaaaaaaaaattagtcgggcatggtaacaggtgcctgtaatcccagctacttgagaggctgaggcagggagaattgcttgaaccaggtaggcggaggttgcagtgagccaagatcgcaccactgcactccagcctggggcaacagagcaagactgtctcaaaaaaaataaataaataaaataaattcttaagaaggatattttggaaaactccttacatacctaaattctttgtttatcaaatacttggacttagcacactcttctttgaaatggaccaataaacaacaggagcccataagcaaaaagaactcattattttaaaaacagtaactatccttacaggctttctcagggctctttctgttggatccttccctctcacaggtccttgctaatgatctctaggtggacacattctagatgagatgtccctgtctagaatggcagcaccatgagggctatatcctcagtactaggacagcgcctggtgcttaatagatagtaaatagttgtctaattaactgagcaaacagatagattcatgaattagctttttgctttttctgttagaaactaaaggttcaggtcaggcacaatggcgcatgtctctaatcccagcactttgggaggccgaggcgggctgatcacttgaggtcaggagttcaagaccagcctggccaacatagtaaaaccctgtttctacaaaaattaccaaaattagccgggcgtcttggcaagcacctgtaatgccagctacttgagaggctgaggtgggagaatcgcttgaacctgggaggaagaggttgcagtgagccgagatggtgccaacctgggtgacagagggagacttaaaaaaaaaaagaaagaaagaaagaaaagaaactaaaggttcaaagaatcccagaaaaggaagagtcctcacaagccagtaatctaggcaggattactgatagtatttttatatttgttgtatttttataaaatgccatagatagagggcttttttcaacattacatcagtctaaaaatcacacatttttatatgaactaacctaaatgtctgatgaatctcacaacaccaagtctttgaaatgtgcccatataaataaaatgttaacagattcatgctaattttaaatatcgatagtgtttaaatgccttaattattttttcactccctagctttaaaagaaaataaccaacttcaaaaggacatcacaataacatcaagtctatttgggggaatttgaggattttttccctcactaacatcatttggaaataatttcatgggcattaattgcatgaatgtggttagattaaaaggtgttcagctagaacttgtagttccatactaggtgatttcaattcctgtgctaaaattaatttgtatgatatattttcatttaatggaaagcttctcaaagtatttcattttcttggtgccatttatcgtttttgaagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaaggtgtgtattgttggccaaacactgatatcttaagcaaaattctttccttcccctttatctccttctgaagagtaaggacctagctccaacattttatgatccttgctcagcacatgggtaattatggagccttggttcttgtccctgctcacaactaatataccagtcagagggacccaaggcagtcattcatgttgtcatctgagtacctacaacaagtagatgctatggggagcccatggaagatacatggtatacaacatagctcttgctctattggaagctaagtggaatgggagaaattggtgacaggcaaccccataatttcagaaagctatgaaaaagtactcagacatattccttataacactggtgtcacatcacaaagacctatttaatgtgcttctgatttatagggagagacatcctatacttcaggaactgcactttgatccacagaaagcctagtgatgtagagctcctgttagttcaaaaggaaaagaaaagaacaacacagaaagcctaattatgcaatagagtcaagtgctttatagcaatgttacagttatcaaaaaaaatccagatggacctctgagaggatgccattggagtaaccaggcagatgcagttgatcagagctgacttcctataagaagtgagcactgagctgaggaataatggcataaatgaaggaaagtgagatggaaatttgagtttttaattggaaagacaatacatcaggcagatttttaaataggggcaaacaaacagacacataggagatgctaggcatggggtccccactaggatgctgcttagaaacatgcaggggtggtgagtactcccaaagtacacttcattcctagctcagtgattcttatctgagtgttaaagttccttcttcagcaccccgttccacagtccaactgggaactttaagacctttcttggagtctttctaggaactcaagtctgctacttatacagaacagtggctttggtccccagttgtgccttgcagtatttttgtgttcaggaagaaacagtagctcttggataaagaagctagctagaaactctgttgctatggcagtgcttcaaaatgtatttccttaaatgctttctttgtaactatcttcatttagttcatctctcagataatgagagatcagagtcccatccccagtataatactcttctttagggtactttcaccatcttcagtctaaacacagactagactttcaattataatgtgtaagatttaaaatgttattattgtgtgactttgaatatctgtgtaaatctactatctcctctttggtatatacgtgtgtttatttttttctggagatctgtaactgaaatgcttaatttctgaattgttttggatatcacaacttaataccaacataagttttgagcctttttctccctaaatctggtgtgagtctaactgaaactcaaatgaactttttaaaaataattttttcttttctttaattttttttttaagtagagacagggacgcactgttaactaggctggtcttgaactcctgatcttgagccatcctccccgacctgagcctcaccttatagagagggtcttgctctgttgcccaagctggagggcagtggcataatcacagctcactgcagcctctcgacctcctcaagcgatcctcctgccttagcctcccaagtagctgggactataggcgtccaccaccatacccagctaatttttttttttattttttgtagagacaaggtctccctatgttgcccaagttggtctcaaactcctggactcaagcagtcctctcacctcagcctcccaaagtgctggggttacaggtgtgagccatggcacctggccagaacttctagtaaaaagaatattgttgccgggtacggtggctcacgcctgtaaccccagcactttgggaggccaaggcaggcgaatcacctgaggtcgggagctcgagaccagcctgaccaacatggagaaaccacatctctactaaaactacaaaaaattagccgggcgtggtggcacatgcctgtaatcccagctacttgggagctacggtgcctggcctagtttattatttcttaatatctgttgtcttccagtgtcttccttaattcttcacaataccctgtacaatgcttagcacacagtgggcagtctgtaagtttattaaatgtttggtgtggcccatacttcctatccacaaagaatgtaacatgttaagacatctagatgagggaatgatttaagaggaactacaataatattctgaaacttggactctggatctctgcatttagactttcctaaaccagccagcaagtagatcatcatgtcacaaggcttaggttgggcttgctgttcagagaatgaattaaggattaaggagaaaaaaaagcagaaaggttttgctctgtttttcaggttctattgagttgttaacttctaacaagttatcttatttgcttcattgcatgaggcccattgtagtaagaagaggaatttatatgctaaatgttctggtgatagaatgacttttctttttttttacagtccaaaggtcttttttttttttttttaacacctattatgccatgaattcatagggaataggttccagctgctcaggctccttcccattggttctcacaaagtgtgcttctctgggtggagcaggctggtgcttcagttgaacccacgtacctttctctttggcttctttctttttctgatcattttccttcacgcgtttcaggaagctgtcttggctcttagagtgtttaatgtgctcaatacgcacattaattctcttggcaagaatcttgcccttaacttgtttacagcgatgccaacagcatgctgggtcacgttgtagacttttccagttttgccatggtaacacttgtggggcattcctttttgaacagtacccgttcccttgatgtctacaatttcacctttcttacagattcgcatatacatggccaaaggaacaactccatgttttctaaaaggcctagagaacatatatcaggtgcctctcctctttccctttgtgttcgtcattttggcaaattactgaaagatggtggttctggccaaaaggaggaatgactttttaatagctgtgtttgtatctgagccttccctctgcctttcattttttttgttttgttttgttttgtttttgtttgagatgaagtttcacttttgttgcccaggctggagtgcaatggtgtgatttcggctcattacaatgtccgcctcagcctcctgggtagctgggattacaggcacccgccaccacgcccagctaatttttgtatttttagtagagacagggtttcaccatgttggccgggctggtctcaaactcctgacctcaggtgatctgtccacctcggcctctcaaagtgctgggattgtaggcgtgagccacatcacctggccacttttttaactctttccaatggttaattccgtttgatatggttccttggaacttgcacattaccctttatcaattatcaccctgtattgggggtggggaggatgatacctctcttcatagttagatcctacttactttcaacagagttcttaacaatcctagaaactcacaggtccagaaaagacaagcataaaggaaactataaataatgcatttgaagactaactcaggaaatcaatgattatttccccccaggctacccagtgtcttaaaaaaacagtttaattaatacaatcttttgtttcaattttctacctatatttatggcttttagcttttctaataaaagctcaaaatgaattacagtcatcagtgactttttaatgaatagaagacttttgcaatttttaactatttgtttttacttattaaatatttccgccttggccaggcatggtggctcacgcctataatcccagcactgtgagatgccaaggcaggaggatcacttgagtttaagagttctagaccaggctgggtatggtggctcatgcctataatcccagcactttgtgaggccaaggttggcggatcacctgaggtcaggagtttaagaccagcctggccaacatggtaaaaccccatctctacaaaaaatacaaaaattagccaaggggtggtggtgggcacctataatcccatcttcttgggaggctaaggcaggagaatcgcttgaacctggaggcagaggttgcagtgagccgagatcatgccactgtattccagcctgggtaacagagcaagactctgtctcaaaaaaaaaaaaaagtttgaaaccagcctggtcaacacagcaagacacccatctcgttgaaaaataacggtcgggcgcagtggctcacgcctgtaatcccatcactttgggaggccgaggcaggcagatcacctgaggtcgggagttcgagaccagcgtgaccaacatggagaaaccccatctctactaaaaatacaaaattagttgggcgaggtggtgcatacctgtaatcccaactacttgggaggctgaggcaggagaacagcttgaacctgggaggcagagaggttgtggtgagccaagatcatgccattgcactgcagcctgggcaacaagagcaaaactccatctcaaaaaaaataaataaataaaaataaataaataagtacttctgcctttaagccacttcctagaaggcagtggcacaaagtgatacatttggaggagtaaatatattacaaaatgaattaggctgggcgcagtggctcatgtctgtaatccccgcactttgggaggccaaggcgggtggatcacttgaggtcaggagttcgagactagcctgatcaacagggtaaaatcccatctctactaaaaataccaaaaaaactagctgggcgtggtggcaggcacctgtaatgtcagctactaggaaggctgaggcaggagaatcgcttgaacccaggaggtggaggttgcagtgagccaagattgcaccattgcacttcagcttgggcaacagagtgagactccgtctcaaaaaaaaaaaaagaactaacatgccagaactttgccttcagtatgttttgtgatttttcccttcttgtgccatttcatcattagttccatgtattatttaagatttcttatcaaccagcaccttgggatttttttgtgtatgtgttggtttagggggtttatttgtttttttcttttttttcggtaattgaaaatgtgaagcaaaatgtcacctgttttttctttcatgtctgacactcatgtcttgtttacccccgacatgcagaagctgaaatccccatttcatacagtcttcaatgtggaggcagtagggatggagaaaataatgtactttgtgctctccggtactctttctttcctattgtctgaggggatttgggcataatttattttgctgcagagataaaaatttgttatatatattttttatcattcagggccaaggaatatagattttttttttcagccttgtctcagctgggtgtctttatttactctgtcttaaagtgttccttttattatcattattattttttaatcattgaattccatttggtgctagcatctgtctgttgcattgcttgtgtttataaaattctgcctgatatacttgtttaaaaaccaatttgtgtatcatagattgatgcttttgaaaaaaatcagtattctaacctgaattatcactatcagaacaaagcagtaaagtagatttgttttctcattccatttaaagcagtattaacttcacagaaaagtagtgaataccctataagccagaatccagaaggcctttctgctgacaagtttgaggtgtctgcagatagttctaccagtaaaaataaagaaccaggagtggaaaggtaagaaacatcaatgtaaagatgctgtggtatctgacatctttatttatattgaactctgattgttaatttttttcaccatactttctccagttttttgcatacaggcatttatacacttttattgctctaggatacttcttttgtttaatcctatataggttttttgaacctataacataagctacaacatgagaaatgtgcggttagatagatatgtcccttctgaaggtcagaaaaaaatataatggaggtaaaacctgaacaagcttggaaactgatggtagacttcttcaaggcagcccttgccctaattaaaattcttgtctttctagaaaaagtctagctgttgatttaccacagaaaataataataataattactattattattattttttgagacagggtcgccctgtgtcacctagattgcagtggtgcagtcatggctcactgcatcctccgtttttcaggctcaagcaatcctcccaccttagcctcctgagtagctgggtccacaagcatgcgccacccacacccactaagtttttgtatttttggtagagatggagttttaccttgttgcccaggctggtctcaaattcctggactcaagtagtccgcccgccttgccctcccaaagccagaaaacatttagaatatctttcagagatgtgtatttacaccactattaacacagggctgtatagcagtccagtactggactatgtagtccagtactattcttttccttactggagggccaggcgtggtggcaggtgcctgtaatcccagctactcaggaggctgaggcaggagaattgcttgaacctgggaggcagaggttgcagtgagctgggaccgtgccattgcactccagcctgggcgacagagcaagactccgtctcaaaacaaaaaaaaaaagagagagagagcagtaattcaggtctcacccatcttcaatccagggggcctagccttagtatttgacccatagtaagcacccaataattgtttaaattaattaacctctgaggccctttaaatctgttgataagtatcttattttgcaaagtcctaagcacttggaagagcagaggaactatttactgggtgtgtatgcttttctaacaatattttatagctggcttttgtttttagaatgaatttgaacattgaaaaggcaggcaatagggatgattctgtgaattctgctaaaactgagtagaaagaatgagtgtagagatgtcgacattgatcaactttctatcttcataagagatctgattctaacatatccatttagactcaagtagaatattgtgtatagagtgagtggcagtgagtaatttggtaaaaatttgctgacctgcttttattctttcctcctttctttcttcctttccttccttccttccttccttccgtcctttcctttcctttccctcccttccttccttctttccttctttctttcctttctttcctttcttcctttctttccttcctcccttccttttcttttctttctttcctttccttttctttcctttctttcctttcctttctttcttgacagagtcttgctctgtcactcaggctggagtgcagtggcgtgatctcggctcactgcaacctctgtctcccaggttcaagcaattttcctgcctcagcctcccgagtagctgagattacaggcgccagccaccacacccagctactgacctgcttttaaacagctgggagatatggtgcctcagaccaacccaaccccatgttatatgtcaaccctgacatattggcaggcaacatgaatccagacttctaggctgtcttgcgggctcttttttgccagtcatttctgatctctctgacatgagctgtttcatttatgctttggctgcccagcaagtatgatttgtcctttcacaattggtggcgatggttttctccttccatttatctttctaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctaggtaatatttcatctgctgtattggaacaaacactttgattttactctgaatcctacataaagatattctggttaaccaacttttagatgtactagtctatcatggacacttttgttatacttaattaagcccactttagaaaaatagctcaagtgttaatcaaggtttacttgaaaattattgaaactgttaatccatctatattttaattaatggtttaactaatgattttgaggatgagggagtcttggtgtactctaaatgtattatttcaggccaggcatagtggctcacgcctgtaatcccagtactccaggaggccgaggcaggtggatcagctgaggtcaggagttcaagacctgtctggccaacatggtgaaaccctgtctctactaaaaatacaaaaaaattaactgggtgtgctagtgcatgcccgtaatcctagctactctggaggctgaggcagcagaatcacttgaacccgggaggcggaggttgcggtgagccaagatcacaccactgcactccagtctgggtgacagagcaagactccatctcaaaaaatatatatatatatatatacacacatatattttatttcaactgttagacaagagtccaaaggccaaagaataaagttttaggccagtcctttattagaaaatgagtcaaatcccaaagcaagtttttttatgagttaatgaatataaatgactacatattttatgccttaaaaatcacttttaatgaatggtgttttatggcttgtaaatcagagttttaatcagtaaagaaagtttttaatcctcaaaaacacgttatcataaaagacactgtttggcatcaaatgtggtatttggccatgttcattagggtcattttaggaatctcatacattctacttagctatgcttaattcctgataccatggcattttctgaaatgtttcaaggatgacatctctgctgtttttaatttggtaatgatatctgctgatttattaagtgaaaaaagtaatggtgtcattaccttggatgaagaaacaaaaataaagcatttgccacatttttcaactttgttttcctttcttacaaaattgctataagctcattgcccccaaattggacaatatagggaataaaaaagataatttggggtggggttagacacgggtcttgttatgttgccgaggctggtctctaactcctggcctcatgcaatcttcctaccttggcctcccaaagtgctgggattataggtgtgagccacttcaccaagctgagatgccacctcttaaaagagagaataaggacagattacagccactgctcatgcctgtaatgtcagtactttgggaggccaaggtgggagaattgctcgaggccaagagttcaagaccagcctgggcaatgtagcgagacctgatctctatgaaaaggggggtgggggggaaaactagctggggccaggcgtggtgggtggcttacgcctgtaatcccagcactttgggaggccgaggcgggcagatcacctgagggcaggagttcaggaccaacctgaccaatatggagaaaccctgtctctactaaaaatacaaaattagccaggcttggtggcttatgcctgtagtcccagctactcgggaggctgaggcaggagaatcgcttgaacctgggaggcagaggtttcagtgagctgagatcgcgccattgcactctagcctgggcaacaagaatgaaactccatctcaaaaaaaaaaaaaatcagctggaaggtggcaaacacctgtggtcccagctactcaggaggctgagacaggaagatcacttgagtccaggaggtcaaggctgcaggtgagccatgtttgtgccactgcactgcagcctggatgacagaccgagacccttctcaaaaaaaaaatttttcccggtatttttttttgggggggggtttaattcttgttgcccaggctggggtgaattggggaattttgggttaagggaaccttcggcttcctgggttggggggtttttcctgtttaggcttccccagtagctgggattacaggcatgcaccaccacgcccggctaattttttgtatttttagtagagacagggtttctccatgttggtcagactggtctcgacctcttgacctcaggtgatccgcccaccttggcctcccaaagtgttgggattacaggcctgagccaccgcacccggcctgtactcttattctttaataataaaatatttctgtgtttctttagtcattttacataaacttttatttatttatttatttttatttatttatttttttgagacggagtctcgctctgttgcccaggctggaatgcaatggctcaatctcagctcactgcaagctctgcctcccgggtacacgccattcccctgcctcagcctccctagtagccgggactacaggcgcccgccaccacgcccagctaatttttttttttgtattttcagtagagacagggtttcactgtgttagccaggatggtcttgatctcctgacctcgtgatccacccgtctcggcctcccaaagtgctgggattacaggtgtgagccaccgtgctcggcccataaacttttatttttaaaataatgtcatgataaataatattgcttaggtgtctttaatatattagtaacatttctgttttattgtacatcaacatttatattcaaattaatgggtgaagagtactccattggactaggtatatcgtaatttaatctcctattattggacaactacattgtttctaaaattatactattacaagcattgttacagtgaacattcctatgactaaacctttgcatatatcttttatctccctaggatatatttctaaaactagcattgttgactgaaagtgtaaatacgtgttaaggtgtttgctacataatgccatatttcctttttaggaaactaagctactttggatttccaccaacactgtattcatgtacccatttttctcttaacctaactttattggtctttttaattcttaacagagaccagaactttgtaattcaacattcatcgttgtgtaaattaaacttctcccattcctttcagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttgtgagtgtatccatatgtatctccctaatgactaagacttaacaacattctggaaagagttttatgtaggtattgtcaattaataacctagaggaagaaatctagaaaacaatcacagttctgtgtaatttaatttcgattactaatttctgaaaatttagatctagataaagctatagtgtggattattttatgtatatttacttgagaaaataattattaaatattagtggaaaagctatactttgggtatgatataggactttcgaattggaattttcctttctatctgtaaaagcaagtaggtatagttttattccccagaaggcatctttttctcccccttgtctcacatgggtgaatttaccagcatatttaactaaattcagactggttccaaatgtactgccagatagtagcatttctctagtgtttgttttcatcctggcttgtaagaatgccctgccacttctgccctgcaatatcccttgctattaggattttggcatcaccttgggtccttaatgccagaaatgggaattgcttcatactgtggaaaaatacccattaaaatattaagaccagtaaaacctcgtttctgcttgggctatttgtggatttcagacatcctgagaagtttaccacccctgtaattaattgtcattgtcatcacttcataataaaaataattgcatggccgggcatggtggctcaagcctgtaatcccagcactttgggaggctgaggtggtcagatcacctaaggtcaggagatcaagaccagcctgaccaacatgaagaaaccccatctttactaaaaatacacaattagccgggcgtggtggcgcatgcctataatcccagctactcaggaggctgaggcaggagaattgcttgaacccgggaggcggaggttgcggtgagccgagattgcaccattgcactccagcctgggcaacaagagcgaaactctgtctcaataataagaagaagaattgcgtgaatatttctttaaaactatgatgagataacataccagattatcaaatggattcagtagtgggtgtgccatttattgcacactgagagatgaccaagtcattctgaaatatctttattaatatatccttcctaggatttttcatcctaacttctccataggtagttacttagcataacatctctgtggccagatgtatcccactactaaaagggcaaagtaagctgtggctgccctggtagatacaatgagtaagtgcacagtgatggctataaatgttttcatctcataatcccatgtccagaccagcaatttgctctgaaagctcttacctgtgtctgtttcaatggctcttgatcacttgcctgcacgtccagaattccttatttattcattgaaaattagcgttctttatccctttgttttgcaagttcagctttttagagatggctaaaatggtctaatctttcttggcaaaggcaattctgagctgcagattagactacaagtggcttgggtacatgttgtctttaaacaagcgaagaggaaaactttgagctctattcagacttggtgaagtgtggtaaatttatgatgaaagctactgactgtattacacatgattaattctgaagcccatattaagatgatcttttcagcagttcagcattgctcttctaactgaacagtttcaaggctgggatttcagcaattaatcagttcagaattgctaatgatcctggcggagggtggtagcaaaagggggaggatgtcattagcttctctagcctgccttttttcagtgccctgtggcagtatggagtgaggcaacatgaaagaaagatggcctgaccttcatggcagtattgtgcaacacgtaaatactggtgtgagtggctgtggctatggctagtaaatgatggcccttggtaaacaaagttatttatcagacaatacctaccagctaggtcaactgtgcccataattgatctggttaatttcttttgctgcctattgatttttatttggttgatagataatagctagaggactctaaatttctttggggaagaacatgaaccccttctaagccttcttacgagagaattgatcgcttttgcactgacctttagtaacatcctgatttcagtgttttgtaactatcagagggttgagtcttggttttaagccatgtatatctgtagcataactttctgtgtaggctagttacctctcagcttataaagtgtaggctgataaatttatagtacagtagagtgtcactatgcaaagaaacgatcttagggaatcgaatgatatctgctattaaagcaaaattaatatatattttttctttttacttttttttttttttaaagacatgaaatctcactgtattgcccaggctggtcttggtctcagactcttgagctcaagcagtcctcccacctcagcttcccaaagtgctgggattataggcatgagctgccgtgtctggcccagtatatattttttaagttttaagttttgtggtacgtagtaggtttataatattattttgaatccttagttgtaattttatgtctgctgatgtgtacataatttttattaaactatttatttgagacttcaggtatcttttttttttttttgagacggagtctcgcactctcgcccaggctagagtgcagtggcgccatctcggcttactgcaagctctgcttcctgggttcacgccattctcctgcctcagcctcctgagtagctgagactacaggtgcccgccaccacgcctggctaattttttgtatttttagtagagacagggtttcaccgtgttagccaggatggtctcgatctcctgaccttgtgatctgcccgcctcagcctcccaaagtgctgagattacaggcgtgagccaccgcgcccagccgagacttcaggtgtcttagaattttttaaatgtaccctttctgagaaaaacagagacttaaagctaggataactggtattctattttttttttttttttttttttttacctccagcctgggtgacagagcaagactctgtctaaaaaaaaaaaaaaaaaaattcactttaaatagttccaggacacgtgtagaacgtgcaggattgctacataggtaaacatatgccatggtggaataactagtattctgagctgtgtgctagaggtaactcatgataatggaatatttgatttaatttcagatgctcgtgtacaagtttgccagaaaacaccacatcactttaactaatctaattactgaagagactactcatgttgttatgaaaacaggtataccaagaacctttacagaataccttgcatctgctgcataaaaccacatgaggcgaggcacggtggcgcatgcctgtaatcgcagcactttgggaggccgaggcgggcagatcacgagattaggagatcgagaccatcctggccagcatggtgaaaccccgtctctactaaaaaataaaaaaattagctgggtgtggtcgcgtgcgcctgtagtcccagctactcgtgaggctgaggcaggagaatcacttgaaccggggagatggaggttgcagtgagccgagatcatgccactgcattccagcctggcgacagagcaaggctccgtctcaaaaaaaaaaaaaaaaaacgtgaaaaaataagaatatttgttgagcatagcatggatgatagtcttctaatagtcaatcaattactttatgaaagacaaataatagttttgctgcttccttacctccttttgttttgggttaagatttggagtgtgggccaggcacggtggctcacacctgtaatctcagcactttgggaggccgaggcgggtggatcacctgaggtcaggagttcgagaccagcctggccaacgtgttgaaaccccgtctctactaaaaatataaaaattaggtgggcgtggtggcaggcacctgtaatcccagctactcaggaggctgaggcagcagaatcgcttgaacccaggaggtggaggttgcagtgacccaagatcgcaccattgcactccagcctggggacaagagcgagattcttgtctcaaaaaaaaaaaaaaaaaaaaaaggtttggagggtggtgagctgagatagtcaactattaactcctatctacctgctgggactacactggtgaggtggagcctaagtcctaaaacaacaagtgaggcagctggacgcggtggctcgcatcagtaatcccagcactttgggagcctgaggcgggcagatcacaaggtcaggagttcgagaccagcctggccaatatggtaaaacccagtctctactaaaaatacataaattggctgggcgtggtggtgtgcacctgtaatcccagctacttgggaggctgacacagaagaattgcttgaactctggaggctgaggttgcagtcagctgagatcctgccactgcactccagcctggcgacagagtgagactctgtctcaacaacaacaaaagaaagaacaagtgaggcaaaacctggagaccccagcttcatgtaacacctagtttgagtattgttgagagtttttcaggaaaaaagtctgataacagctccgagatagtcttaacatatgaaaaagcaaaaaagggaggagacagatcatttgtcctatacctttctcttttaaggttttaattataacttgtgtaatacaggagacctctgggtgtttttagttgactataaactaaatctgagtacacatttcagggctgctaaaaatgcttatttgaaactgggccgtattaacacaagcagaggctctggagcaagtgaagtacagatccagagccccactgtattctccaatggagtgattgcctgaaagatgatgtcagttttaagcaccgtgcttggtttttaacatggtcactgacaaattggagagtgtttatccagaggtagatggtaaagatacataaaagtaacttgaaatactgtcttttgaagaagaaatgagaagatttaaggaaataagacactgtcttcaagtatctgaagaaccgttacccggaagagaactgttatctggaacaggattaagactcactcatggggctccagaaagcagacgagtgcatggaggacgcagaagatgcagattgtgtggctcaactctaaaatctttctaacaaaattagttctctggatgtgttccagttcacttgatgatgattcttttgtttttgtttttgtttttgaggtgtagtttttcactcttgttgcccaggctgctggagtgcaatggcacgatcttggcttgctgcaacctccccctcccgggttcaagcgattctcctgcctcagcctcccgagtagctgggattacaggaatgcaccaccatacctaattttgtatttttagtagagacagggtttttccatgtcagtcaggctggtcttggactcccgacctcaggtgatccacctacctcggcctcccaaagtgctgggattacaggtgtgagccatcgcgcctagcctatgatgattcttttcacagagatacaggcacttaaggagaggatctaaaccccttggacacattgccgttgaacttctaagatcttaggtttccacttactcatgaaaattataccacagggtcagagggtagtgttcattggagccaggtgccagaacaagttattacaaactactattttagagaaaaatgtcattaaagtttaagataccttaagctataggtttgcatcaaagttaatgaaaggtaaaaagatgccaagcgtggtggctcaggcctgtaatcccagcgctttggggggccaaggcgggcagatcacgaggtcaggagatcgagaccatcctggctaacacggtgaaaccccatctctagtaaaaatacaaaaaattagccgggcatggtggcgggcatctgtagtcccagctactcaggaggctgaggcaggagaatggcatgaacccaggaggcagagcttgccgtgagctgagatccagccactgcactccagcctggctgacagagcaagactgcatctcaaaaaaaaaaaaaaaaaaaaatgcaaatcaaatctaaagtagttcagtctttaaactcaaagccaatacatttgctttgaactacaaatgaactgaagtttttaagtgtaataaatgttactaaatcggcttttgtagcagttaaacaaaaaacttcaaaaattgtaaggattctgtgagggagcatggctgctgctgctgctgctgcttgcagatagcctgctgtgtttaggatttagttaaatacatttctcctgtttaaaactaaatggtctttccttagtttgcttagttcttcagaagggcctttgaaacactgggaaataaacaagtgattctttagctactgctttctgaaatacttatataaaagctctgcactgtattctcccatccctctcaggggaatattagagggttaggactccccaggtagacattctaggggtgaaaatttgtcattacattgacatttcagatttaggttttcaacaatactgttttcttctttcacatattgccatctagtaatatagatgttctccgtccacattaatcaaaactattgacatggataattcctaattccttgaacactataatggagatctatagctagccttggcgtctagaagatgggtgttgagaagagggagtggacagatatttcctctggtcttaacttcatatcagcctcccctagacttccaaatatccatacctgctggttataattagtggtgttttcagcctctgattctgtcaccaggggttttagaatcataaatccagattgatcttgggagtgtaaaaaactgaggctctttagcttcttaggacagcacttcctgattttgttttcaacttctaatcctttgagtgtttttcattctgcagatgctgagtttgtgtgtgaacggacactgaaatattttctaggaattgcgggaggaaaatgggtagttagctatttctgtaagtataatactatttctcccctcctccctttaacacctcagaattgcatttttacacctaacgtttaacacctaaggtttttgctgatgctgagtctgagttaccaaaaggtctttaattgtaatactaaactacttttatctttaatatcactttgttcagataagctggtgatgctgggaaaatgggtctcttttataactaataggacctaatctgctcctagcaatgttagcatatgagctagggatttatttaatagtcggcaggaatccatgtgcagcaggcaaacttataatgtttaaattaaacatcaactctgtctccagaaggaaactgctgctacaagccttattaaagggctgtggctttagagggaaggacctctcctctgtcattcttcctgtgctcttttgtgaatcgctgacctctctatctccgtgaaaagagcacgttcttctgctgtatgtaacctgtcttttctatgatctctttaggggtgacccagtctattaaagaaagaaaaatgctgaatgaggtaagtacttgatgttacaaactaaccagagatattcattcagtcatatagttaaaaatgtatttgcttccttccatcaatgcaccactttccttaacaatgcacaaattttccatgataatgaggatcatcaagaattatgcaggcctgcactgtggctcatacctataatcccagcgctttgggaggctgaggcgcttggatcacctgatgtcgggagttcaagaccagcctgaccaacatggagaaaccccgtttctactaaaaatacaaaattagccgggcttggtggcacttgcctgtaattccagctactcgggaggctgaggcaggagaatcacttgaacctgggaggcgggggttgcagtgagctgagatcgcatcattgcactctaacctgggcaacaagagcaaaactccatcaaaagaaaaaaaaaatcgggtgcagtggctcatgcctgtaatcctaacactgtgggaggccaagacaggcagattgcctgagctcaggagttcgagatcagcctgggcaacatggtgaaaccctgtctctactaaaatacaaaaaattactcagcgtggtggcatgcgcctttagttccagctactcaggaggctgaggcaggagaatctcttgaacccgggaggtggaggttgcaatgagccaagatcgtgccactgcactccaacctggcaacagagcgagactccgtcttaaaaaaaaaaaaaattttgcagcgcaaaccaggatatcctctgttctcatttgttctagatttcaaaagaaacagtcctttctttggggaaaagagaaaggaaaaggagttttataaaaggaaagaaaagattcataagaacaagaagtgggcccacttgcatatacctttgtagaaaactgttcactgttgttgaagaaaagctcttcatattaatatgcagtccagatgcagtggctcacacttataatctcagccctttgggaggctgagacaggaagattacttgaggccaggagtttgaaaccagcctgggcaacatagtgagactctgtctccacaaaatttttttttaattagccgggcatggcagtgtgcttctgtagtcttagctactgaggaagctaagccagaagaatcacttgagcccaggagttcaaggctgcagtgagctatgatcataccattgcactcttgcacttgcacagagcaagaccctgtctcttaaaaaaaaaaaagtgtgtgtgtgcatatgcatatatacatatatatacatgcaaatgtatctgtttataattcagattgcttcaaaaagatgttgcactttatgatactgagaacagtgagaagtaaataagatagagtgtaggaggaggaataatttcagaacagccatctgagaacttctgtgacaacagatcaggcaaaatgaaatgtgaaagtaattttataggccaggcgtggtggctcatgcctataatcccagcactttgagtggccaaggcaggtggatcacttgaggtcaggagttcgagaccagcctggtcaacatggtgaaaccttgtctctactaaaaacacaaaaaaattagtcgagcgtggtggcatgtgcctgtaatcctagctgctggggaggctgaggcaggagaatcacttgaacccgggaggcggaggttgcagtgagcctagattgcaccactgcactccagcctgtgagacagaatgagaccctgtcttaaaaaaaaaaaaaaagtaattttataaactattgtgcacaattcgatgtattcataattaattaaatgattatttttgttggttttaacttttattcagtggctatttattgggagcctactgtgttctgggcactaggaatgcaacagtaaataagactaactaagtccctggtaggattcaggttctgtcgaggggagatacacaataaagatgaatttaagataacaataaatgctatggagaaatatacagaacagtggaatagtattagctgtcaaaggttgttgattactttcgtttaaggaggccagggaaagcctttctgaaaaaattgagctgagacctaaataacaagaaataattgtccttgaaaaatgaagggaatgcatcttataggcagaggaatagcaaacataaaggtcttgaggtaataatgagtgtggttttttgatttctgtattttggtttttttgagatggtgtctccctctatcccccaggctggagtgcagtggcacaatcttggctcactgcaaactctgtctcctgggttcaagcaattctcctgccttggcctcctgagtagctggtattacaggcacgcgtgctaccacacccgactagtttttatttttagtagagatggggttttaccacgttggtcaggctggtctcaaactcctgaactcaagtgatccaaccacctcaacctcccaaagtgctgggatcacaggcgtgagccaccatgcccggccagagcttggtttattttttaaaagataggccaatgttggtcgtgtgtggtggctcgtgcctataatcccagcactttgggaagccaaggcaggcaaatcacttgaggtcaggagttcgagaccagcctggccaacatggtgaaaccccatctctactaaaaatacaaaaaactagcatggtgtggtggtgtgtgcctgtaatcccagtgcctgtaatcccagctactccagaggctgaggcaggagaatcacttgaaccgaaaggtaggagttacagtgagccaagatcgcatcactgcactccagcctgaacgacagagcaagactcctgtctcaagaaataataatgataaaaggttcgggcacagtggctcacacctgtaattccagcactctaggaggccgaggcaggcagatcccctgaggtcaggagtttgagaccagcctggccaacgtggcaaaaccccatctctactaaaaaatgcaaaaattagctgggcacggctgggtgtggtggctcattcctgtaatcccagcactttgggaggtcaaggcggacagatcactgaggtagaaaccctgtctctactaaaaatacaaaaatttgcccagcgtggtggcgcgtgcctctaatcccagctacacgggaggctgagacaagagaatcacttcatcaacccgggaggtggaggttgtggtgagctgagatcgcaccattgcactccagcctgggcaacaagagtgaaactccatctcaaaaacaaaaaaaaattagctgggaatggtggcatgtgcctgtaatcacagctacttgggaggctggggcaggagaatcgcttgaacccaggaggcggagattgcagtgagctgagattgcgccactgcactccaggctgggcgaaagagcaagactccgtctcaaaaataataataataataataataggccagtgtagctggagtaatttgcaaattatgtgtggaggcagagattacacaaggaatgggagaaggtcatagatgagggccagatcacatagtatttggtggtaaggaattcagattttatccttgtggtaattggtggtgtggagatggttaaaaacaaggttggtttgggatgggtttgaagagaggacttgctaatggattaaatttggaggataaggtaaagagaaattgaaggagtgacacttgggttttggcttgaacaatagatcttgttagtaatattaaattagatgaagaaggcatggtagggaatatgggggagtgggaaaggcaggaagcaggaatggaaccaggaactctgttttagatgtgagaatttgttgttgttgttgttgttgttgttgttgttgttgttgttgttgtgacagcatctcgttctgttgcccaggctagagtgcatggagtgcggtagcacgatctcagctcactccaacctccgcctcccggttcaagtgattttcctgcctcagcctcccgagtagctgggattacaggcacctgccacaatgcctggctaatacttgtatttttagtagagatggggttttaccatgttggccaggctggtcttaaactcctgacctcaggtaatccacccacctcggcctcccaaagtgctgggattgcaggtgtgagccactgtgcccggccagatgcatgaattttgagatgtatactagacttctggatagagaagttaagtaggcagttggacacattgtatgaagctcaggggtacaaggaggactatgaacatgggagtcttctgacaaatttatcactagactcctcattcaagtaactaggaaatgtcagatattcttcccctagtaatagccagtggttatactcttgcctttagttttcttcacaatactcttggcaacacataaggccttccctacaatctgagtttcagtcagaattgtttctgagcgttcttcctcaaatttctccccagtctcattattctttattctcatgtccatgaccagtcataatagtaattatgaaaaacctctaactttctttagtgcattgaatgtatattttatcattttggttgtgttaactgtaaatctctcagtggaaatctgaaaagcctttatttccttagatgataatatacaattgatttaggagatagggaatttttcagttacctttataacagcacagtattagcagtctaatctaaatgctaagtgaatgttttgagaggagatagatgttgaaaattaaaatacattaagtcccagtgaggtgaaaagccgattgttaagttctgcacacaaaagatttgcttcagtgaattgatttcaacagctgagatcctagtcatttcacctggtctaccaaaaagaatgattttacttgcttttggtcaaatctctgcccagcaattctttttctttctttcttttttttgttttatgtgtgtgtgtgtgtgtgtttttttttagcagagtctcactttgtcacccaggcgggagtgtggtggtatgatcacagttcactgcagcctccaactcctgggctcaagtgatcctccagcttcagcttttcaagaaattgggactgcaggcacatgcaactatgcctggctgaggttttatgtatcttttttctagagaaggggtctcactgtgttgcccagctgggtctccagctcctggtctcaagctgtcctcctgcctcagcctcccaaagtgccaaagtgctagggttataggtgtgagccattggtgcccagctactgcctgcctggcaattctgaatgccttaaattttttttttttttttttttttttttgagacagagtttcactctgtcacccaggctggagtgcagtggcatgatcgtggctcacagcaacctctgcctcctggattccagcaattctcatgcctcagcttcccgagtagctgggactacaggtgcatgccaccacgcccagctaatttttggtttttttgtttgtttgtttgtttgttttgagacggagtctcgctcagttgcccaggctggagtgcagtggcgtgatctccgctcactgcaagctccgcctcccgggttcacgccattctcctgcctcagcctcccgagtagctgggactacaggcgcctgccactacacccggctaatttttttgtattttaagtagagacggggtttcaccgtgttagccaggatggtctcgatctcctgacctcgtgatccgcctgtctcggcctcccaaagtcctgggattacaggcgtgagccaccacacccggcctaatttttttttttttaattttatttttaattttttgagatgcgagatggagtctcgctctgttacccaggctggagtgcagtggcaccatctcagctcactgcaacctccacctcctgcattcaaaagattctcctgcctcagcctcccaagtagctgggattacaggtgcctgccaccacgcccaactaattttttgtatttttagtagagatgaggtttcaccatgttggtcagactggtgtcgaactcctgacctcaagtgatctgcctgcctcagtctcccaaagtgctaggattacaggggtgagccactgcgcctggcctgaatgccttaaatatgacgtgtctgctccacttccattgaaggaagcttctctttctcttatcctgatgggttgtgtttggtttctttcagcatgattttgaagtcagaggagatgtggtcaatggaagaaaccaccaaggtccaaagcgagcaagagaatcccaggacagaaaggtaaagctccctccctcaagttgacaaaaatctcaccccaccactctgtattccactcccctttgcagagatgggccgcttcattttgtaagacttattacatacatacacagtgctagatactttcacacaggttcttttttcactcttccatcccaaccacataaataagtattgtctctactttatgaatgataaaactaagagatttagagaggctgtgtaatttggattcccgtctcgggttcagatcttagctgataagtggaagagctgggactttaagcagatgagaatctaaagactttgctcttttcacttcactggggtgtctttctctctctctctcttgctctctctctctctttttttttttcccaagacggagtctcactccattgcccaggccagagtgcagtggtgcgatctcagctcactgaaaactcatcttgcccaggctggtcttgaacccctgaccttgtgatcctcccgccttggcctccccaagtgctgggataggcgtgagccaccgtgcccagccaataatagctaaaatttatataatgttcactgggccaggcacagcggctcgttcctgttatcccagcactttgggaagctgaggcaggcagatcgcttgagccaaggagttcgataccagcctgggcaacatggcaaaaccccatctctaccaaaaaaaatatacaaaaattagccaggcgtggtggcatgtacttgtagttccagctactcggaaggctgagttgagagtatctcttgagcccaagaagaggggactacagtgaacggagattgcgccactgcactccagcctagacgacagacagaagatctcaaaagaaaaaaaaaaaaaaaagatcactttatgctgggactgctctaaaggcccaaccatgttttaactaattaacaattttatgacaactctatgagctatgtactgtaattatgcctatattacagatgtgaaaattgaggctcagagaggttgaataagttgctcaaagtcacacaggtaataagtgatggaactagaagttgaactcaggaagtctagctccaagtctaaattctttgttaatttatttttcgggccagagtcttactctgtcacccaggctggagtgcagtgccactatctctgctcactgcaaccttcacctcccaagttcaaaccttgttcaattcttgtgccttggcctcccaagtggctaggattacaggcatgtgccacaacaactagctaattttttgtctgattctgttggccagtctggagtgcagtggcgcaatctcagctcactgcagtctccagctcccaggttcaagtgattctcgtgccttagcctcccaaatagctgggattacaggcacgtgccaccacaccgagatagttttttgtatttttaatagaaacaaggtttcaacatgttggccaggctggtctcaaattccagacctcagatcatctgcccgcctcaggctcccaaagtgctgggattacaggcatgagccactgcacccggccttaatttttatatttttattagagatggggttttgccatgttggccaggctggccttgatctcctggcctccagtgatccacccgccttggcttcccaaagtgctgggattacaagcatgagccactgcacccggcctccaattctaaactcttaacaacaatactatagtttcttgaaaagttgttgaaggcttcacggagggaaaaaaaatggagcattctaacaactttgcagatgagacccaagaagactcaatgactttctcctgatcatattgtagcagatgacttagccagaactctgacttcctcacagggagaaagtctgcaagatttcacacttacctgtcaggcctgagctggctgctttctcagctccctaagtgctatgttcccagtctgcttttcttcctttttcaagtgtgcactaccaggcatttcagaacatcccaggctggtcgcggtggctcacacctgtgatcccagcactttgggagcccaaggcgggtggatcacctgaggtcaggagttcgagaccagcctggccaacatggtgaaaccccatctctactaaaaatacaaaagttaactgggcgtggtggtaggcacctgtaatcctagctcaggattactcgggaggctgaggctagagaatcggttgaacccaggaggcggaggttgcagtgagccaagattgcgccactgcactctagcctggggacaagagggagacttcatctcaaaaaaaaaaaaaaaatcccagctgggcacagcggctcacttctgtaatcccagcactttaggaggccaaggcaggaggatcacttgagcccaggagttcaagactagcctgggcaacatagtaagaccctgtctctacaaaaaaatttaaaaattaattgggtgtcgtagcacactcttgtattcccagctactcaggaggctgaggtgagaagaatgcttgagtctgggaggtcgaggctgcagtgagccatgatggtgctactgcactccagcctggccaacattgtgagaccttgtctcaaaacaaaacaaaacatccttctactgagcactttctgtccctttatagaaacttaagagggaaccagtagaggtaatttcctaaggaaaactgctttgggacatgatcacaaatgaagcctggagttttgaactgctgaggtcagcctgtttttaccttctgagcctatcaagtaattgttccagatgccaagaaaagctgctggccttatttctgcttctgcctttaccacaggggagcgccatgtgagccagtcctctgtttttcctccactgtatgctaggcagtattagcaccagattcttcccctctttaaaaagaaattctagtgctttggattttttcctccatgcagaatagcaatgatggaaagtatgtggtcaaagtaatgacattctgaaaatactaaatgtcaccatagtatttttctctggaagagaaatgtatatgtagaggtgaaacttcaaatttctttttttttttttttaagacgaagctttgctcttcttgcccaggctgaagtacaatggcgtgatcttggctcaccgcaatctctgcctccagggttcaagtgattctcctgcctcagcctcctaagtagctaggattacaggcatgtgccaccacgcccagctgattttgtatttttagtagagatggggtttctccatgttggtcacgctggtcttgaactcccgaccccaagtgatccacccacctcggcctcccaaagtgctaggattacaggccaccgcgcccggcctgaaacttcaaatttctttttttttttgagacagagtctcgctatgtcacccaggctggagtgcagtggcgccgtctcggctcactaccagctccactccacctcctgggttcacaccattctcctgcctcagcctcccaagtagctgggactacaggtgcccgccaccatgcccagctaattttttgtatttttagtagagacgggttttcactgtgttagacgggatggtctccatctcttgacctcgtgatccgcctgcctcagcctcccaaagtgctgggattacaggcgtgagccactacgccaagcccgaaacttcaaatttcttatctcataactaggcatcctcatcactgagtgttagcctggatataaacattcctaatcttttgtacttttcatgtcagcatttggctccacttggctgcctggggagaacttctagcattatgagcatgcaggtcctatcaacaggttgggggtgcggtttattcatacaggtagtgagagtggcacagatggatgctgtcccttaaaacaaacagacttgtctttgggagcctgaggcgggtggatcatgaggtcaggagttcaagaccagcctggccaacatagtgaaaccccgtttctactaaaaatacaaaaaattagccgggtgtggtggtgtgcacctgtaatcccagctactagggaggctgaggcaggagaatcacttgaacccaggaggtggaggttgcagtgagccgagatggcaccattgcactccagcccaggcgacagtgcaagactgcgtctcaaaaaaaaaaaaaaaacacacagacttgtcctactgccatttcttttcactctggcggtaaagtaagagtgtgtgtgtgtgtgtgtgtgtgtgtgtgtgtgtgtgtgtgtgtgtgtgtttctgtccgtctgtctgtcaagggggagggtgaccactttctaaaaggccatccgtgtatttttagcttcctgatttttttctctatcgcagtctctttgaagccaggtgaattttaggccttggcaattttctttttattgcaatgggaaggtcaagacactgagagtcacccaaaacatatccatccaaaatgatacaattttagggtttatttttaagtgatacccaagttatttgctaagaacctatgccagtgtgtttatgagaatttgcactgtcccacactgttgccaccagccacatgggactgtttaaatttaaattttacaaattagccagtcatggtggtgtgcacttgtagtcccagctacttaggaggctgaggcaagaggattgcttgagcccagaagttcaatactgcagcaagctatgatcgtgccactgtactccagcctgagtgacagaatgagacctcatctcttcaaaaaaaaagaaaaaaattaaaatatgaagtttagttcttcattcaccctaaccacatctccagtgctcaataactatatgtgactcatggctaccttattagcatagatatagaacattgtgactatcacagaaagttgttttgaacagtgttgccaagccctgtaagtggaagaggcagtgcagtgtgatctgtgtcttcaggaaaccaggtagtcagactagttcaatgaggagaggcagaacctggcttcacttctagattaaaaactgcttaggtggcctaaagatacaatggccattctcagagtagtgagaaggaaggaacagatgtttagggggctagaagaaagtcagagagggccgggcgcagtggcttatgcctgtaatcccagcactttggaaggccaagacaggcagatcacgaggtcaggagttcgagaccagcctgaccaacatggtgaaaccctgtctctactaaaaatacaaaaattagccgggcgtggtggtgcgcgcctgtaatcccagctactcaggaggctaaggcaggagaatcgcttgaaccctggaggcagaggttgcagtgagcccagatcgcaccactacgctccagcctaggtgacagagagagactccgtctcaaaaaaaaaaaaaaaagtcagaggagacaaggagcatgtacacctaaaatcaacatagacccctctgttgatggggtcatagtgagtacttgaggtaccaagtctggataaacatcaaacttcagccaataactttgagtttctagccatccaagcctcttattaaacatacagaaggaccttttttcccttgcatctaacaagttaaagcacctgcagagatcattagggaggagccttggcctgattggtgacaaaagtgagatgctcagtccttgaatgacaaagaatgcctgtagagtgcaggtcaactacatatgcacttcaagaagatcttctgaaatccagtagtgttctggacattggactgcttgtccctgggaagtagcagcagaaatcatcaggtggtgaacagaagaaaaagaaaagctcttcctttttgaaagtctgttttttgaataaaagccaatattcttttataactagattttccttctctccattcccctgtccctctctcttcctctcttcttccagatcttcagggggctagaaatctgttgctatgggcccttcaccaacatgcccacaggtaagagcctgggagaaccccagagttccagcaccagcctttgtcttacatagtggagtattataagcaagatcccacgatgggggttcctcagattgctgaaatgttctagaggctattctatttctctaccactctccaaacaaaacagcacctaaatgttatcctatggcaaaaaaaaactataccttgtcccccttctcaagagcatgaaggtggttaatagttaggattcagtatgttatgtgttcagatggcgttgagctgctgttagtgccaacatgttagtgagaaaatatctttggataggtaaaaatcaaggaggagttctcctcttcctaaaccatcttaatttacttacatagaagaaagcacagcagctggcccaccacggacgggcccagagcaggggaagattctcggtgaacatttctttttttttttctttttttttgaggtcgagtctctgttgcccaggccagagtgcaatggcgcgatctcggctcactgcaacctccacctcccgggttcaagtgattctcctgcctcagcctcccaaatagctgagactacaggcgtgtgccaccacgcccgactaattttttgtattttttttttagtagagacggggtttcaccgtgttagccaatatggtcccgatctcctgacctcgtgatccacccgcctcagcctcccaaagtgctaggattacaggcatgagccactgtgcccagccctctccatgaacattttctaattaaacttgacacttaatacaatgttatgcttaggactgctataaagcttacctctggagttgcgcagcacaaaggccttggtgtgtgtataaatttggtttgttcttttcacagcaaaagctacccacctttgcctcctgtgcctgcttctgcccagggacttaggtcctcttacaccttagagaaaggccttagcatctggtcacaggcagatgagtgacagcaagaaaacctggctgcaatgtaattttgtttccatcctctttattagttatcaattggatttttatgaaatttccaagttccactcaaggatttctcagtgtttttttactttggtatagtggaaaccagggttgccagaaagtattattttgggggtgagttagtcaaccttcgttcagtcagacagacaggagcacctcagcaattcccagaaacgggctgatgggaaagagcaacatacatgaatgtcttgaagaacacagccaacagagcccattgggcagttctgattttccaggtacacagcatctccacagtctcttctgatttttattcccctgagtatatggattccagctcagcatgtagcctttccctgctgagtctctaaccaggataacatgtatttttttgactggatgaattatcttcccatctcttgacatttacagtaattaccaccaagtatggtattttcagtggccgtgattatcagttaccaacacagaattaggatgaagggaggaagggagggaaggaaggtgggtgttttttcacacagtgtcttagccagcaatttagcaaattaatggaaattagatctttgatttttttttctttcaagcattttatttgagagactatcaaaccttataccaagtggccttatggagactgataaccagagtacatggcatatcagtggcaaattgacttaaaatccatacccctactattttaagaccattgtcctttggagcagagagacagactctcccattgagaggtcttgctataagccttcatccggagagtgtagggtagagggcctgggttaagtatgcagattactgcagtgattttacatctaaatgtccattttagatcaactggaatggatggtacagctgtgtggtgcttctgtggtgaaggagctttcatcattcacccttggcacagtaagtattgggtgccctgtcagagagggaggacacaatattctctcctgtgagcaagactggcacctgtcagtccctatggatgcccctactgtagcctcagaagtcttctctgcccacatacctgtgccaaaagactccatctgtaagggatgggtaaggatttgagaactgcacatattaaatatactgagggaagactttttccctctaactctttttcccatatgtccctccccctcctctctgtgactgccccagcatactgtgtttcaacaaatcatcaagaaatgatgggctggaggctgggcatggtggctcatgtctgtaatcccagcactttgggaggccgaggcaggtggatcacttgtcaggagtttgagaccagcctggccaacatggtgaaaccccatctgtactaaaaaaaaaaaaacaaaaagtagccaggcctggtggagcatgcctgtaatgccagctatttgggaagttgaggtgtgagcatcgcttgaacgtgggaggcagaggttgcagtgagccaagattgcaccactgcactccagactgggtgacagagtgagactttgtctaaaaaaaaaaaaaaagagagagagagaaaagctaggtgcggtggctcacgcctgtaatcccagcactttgggaggctgaggtgggcagatcacgaggtcaagagatcgagaccatcctggccaaccaacatggcgaaaccccgtctctactaaaaatacaaaaattagctgggcgtaggggcgcacgcctgtagtcccagctacttgaggggctggggcaggagaatcgcttgaaccccggaggcggaggttgcagtgagccaagatcgcgccactgcactccagcccgggcgacagagcgagactccgtctcaaaaaaaaagagagagagagaaatgatgggctgggccagtgccccacccctgtaatcacaacactgggaggccaaggtgggagaatcgcttgagcctgggagctgaagaccagcctgggcaatacagtaggacctcatgtctacaaaaaaattattaaaaattagccaaggctgggtgcggtggctcatgcctataatcccgggggtgaagttgagcccaggagtttgagaccagcctgggcaacatggcaaaaccctgtctctaccaaaaatacaaaaaaattagccaggggtggtggtacgtgtctgtagttccagctacttaggaggctgagatggaaggattgcttgagcccaggaggcagaggtggcagtgagctgagatcacaccactgcactccagcctgggtgacagagcaagaccctgtctcaaaaacaaacaaaaaaaatgatgaagtgacagttccagtagtcctactttgacactttgaatgctctttccttcctggggatccagggtgtccacccaattgtggttgtgcagccagatgcctggacagaggacaatggcttccatggtaaggtgcctgcatgtacctgtgctatatggggtccttttgcatgggtttggtttatcactcattacctggtgcttgagtagcacagttcttggcacattttaaatatttgttgaatgaatggctaaaatgtctttttgatgtttttattgttatttgttttatattgtaaaagtaatacatgaactgtttccatggggtgggagtaagatatgaatgttcatcacaaaaacataaatcaaggccgggcatggtggctcatgcctataattccagcactttgggaggtcaagatggaggtcaaggtgggagcctagaagttcgagaccagcctgggcaacataaggagacttcatctgtacaacaaatttaaaaagtagctgggtgtggtggcagatgcctgtagtcgcagctacttgggaagctgaggtgggaggatcacttgagctcaggaggttgatgcttcagtgagccacgatcacaccactgtactccagcctgggcgacagagcgagaccgtgtctcaaaaagaaaaaagaaagtataaatttacacaaaaacaataaaataatcccagtaattccaccacttggagatgatcaccataaaactccaccaggcatatgtgcgtatatatacacgtgtattttataaaatgtgatcataattacactgttttgcttttttccttaagatattacatacatttttccacatcgttaaattacagtgctgttttcctggtggctttcctttaacagattgaagttcatgttaatacagttgccagaggctgtgggctttcactgtcaccaggagtcactcctagggcctcttcagagcaaggccttatgtcctgaagcattgccttttttttttttttttgaggtggagtctcactctgtcacttagcaggctggagtgcagtggcccagtcttggctcactgcaacctccgcctcctgggtttaaatgattctcctgcctcagcctcagggcggatcacctgacatcaggagtttgagaccagcctggccaatatggcgaaaccccatctctactaaaaatactaaaaaaaattagccaggcatggtggcacgcacttgtagtcccagctacttgggagactgaggcaggagaatcgcttgaacccaggatgttgaggttgcagtgagctgagatcacaccatcacaatccagcctgagtgacagagtgagactccatctgaaaaaaaagaaaaaacaattagcctggcatggtggcaggcacctgtaatccctgctacttgggaggctgaggcaggagaattgcttgaacccgggaggtggaggttgcagtgagctgagatcgtgccattgcattccaggctgagcaacaagagcaagactccgtctcaaaaaaaaaaaaaaaaaaaaaaaaaggccaggtgcagtggctcacgcctgtaatcccagcactttgggaggccaaggtgggtggatcacctgaggtcaggagttccagagcagcctggccaacattgtgaaacccccgtctctactaaaaatacaaaaattagctgggtgtgatggcatgtgcctgtaattccagctactcaggaggcagagacaggagaattgcttgaacccaggaggcggaggttgaatgagccgagattgcgccatcacactctagcctcggcgacagagcaagactccgtctcaaaaaaaaaaaaaaaaaaattagcttctacctcattaatcctaagaactcatacaaccaggaccctggagtcgattgattagagcctagtccaggagaatgaattgacactaatctctgcttgtgttctctgtctccagcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttccaccatgaatgactgttcttgagacttaggccagccgactttctcagagccttttcactgtgcttcagtctcccactctgtaaaatgggggtaatgatagtatctacctcctaggatttattgaggcagcttaaataccttttgtatttcctgttgctgccaaaacaaattgttgcaaggtcagaagtctgaggtggctcaactgtttctttgtttcaggtttcatgaggccaaaataaaggtgttcgcagggcgtgttcccttctagaggctctgggtccttgcagttctaggactaagatccctgtttcccactggctgttggctgggcatcattctcagcttcttgaggctccccacattcctaggctcctggcctgtctgcctccatcttcaaaaccagcaatgggtggtcaagtttttctcacactgaatcttgctgactactgtatctttctaactcctgccagagacatttctctgtttctaagggctcaagtgattagattgcacccacttggtaatccaaagtgatcttcatatcttaaggcccatagccttaattatagctgcaaagtcccttcgcagcagtacctagattactgttggaatgaataaccagaagacagcaatcaagggaggacatctttagaattctgcctaccacttgtatttaacatgcttaatccacagatgacactctctaccattatttcctggtcctcacactgctcagagattggaatcctttttaagcaaagagaatgaagtcatcacatagttcagtcctgctgtatttgctggaaacagtgaag

SEQ ID NO: 12 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 13 - DNA, 2969 bp, Homo sapiens
ccacgcgtccgctaaaggggaattaatgtttgatgattcttcagattcatcaccggaaaaacaggagagaaatttaaactggaccccggccgaagtcccacagttagctgcagcaaaacgcaggctgcctcagggaaaggagcctgggttgattaacttgtgtgccaatgtcccacccgtcccaggtaacattttgccccctgaggtccggggtaatttaatggctgctggacaaaacctccaaagttctgaaagatcagaaatgatagctacctggagtccagctgtacggacactgaggaatattactaataatgctgacattcagcagatgaaccggccatcaaatgtagcacatatcttacagactctttcagcacctacgaaaaatttagaacagcaggtgaatcacagccagcagggacatacaaatgccaatgcagtgctgtttagccaagtgaaagtgactccagagacacacatgctacagcagcagcagcaggcccagcagcagcagcagcagcacccggttttacaccttcagccccagcagataatgcagctccagcagcagcagcagcagcagatctctcagcaaccttacccccagcagccgccgcatccattttcacagcaacagcagcagcagcagcaagcccatccgcatcagttttcacagcaacagctacagtttccacagcaacagttgcatcctccacagcagctgcatcgccctcagcagcagctccagccctttcagcagcagcatgccctgcagcagcagttccatcagctgcagcagcaccagctccagcagcagcagctcgcccagctccagcagcagcacagcctgctccagcagcagcagcaacagcagattcagcagcagcagctccagcgcatgcaccagcagcagcagcagcagcagatgcaaagtcagacagcgccacacttgagtcagacgtcacaggcgctgcagcatcaggttccacctcagcagcccccgcagcagcagcagcaacagcagccaccaccatcgcctcagcagcatcagctttttggacatgatccagcagtggagattccagaagaaggcttcttattgggatgtgtgtttgcaattgcggattatccagagcagatgtctgataagcaactgctggccacctggaaaaggataatccaggcacatggcggcactgttgaccccaccttcacgagtcgatgcacgcaccttctctgtgagagtcaagtcagcagcgcgtatgcacaggcaataagagaaagaaagagatgtgttactgcacactggttaaacacagtcttaaagaagaagaaaatggtaccgccgcaccgagcccttcacttcccagtggccttcccaccaggaggaaagccatgttcacagcatattatttctgtgactggatttgttgatagtgacagagatgacctaaaattaatggcttatttggcaggtgccaaatatacgggttatctatgccgcagcaacacagtcctcatctgtaaagaaccaactggtttaaagtatgaaaaagccaaagagtggaggataccctgtgtcaacgcccagtggcttggcgacattcttctgggaaactttgaggcactgaggcagattcagtatagtcgctacacggcattcagtctgcaggatccatttgcccctacccagcatttagttttaaatcttttagatgcttggagagttcccttaaaagtgtctgcagagttgttgatgagtataagactacctcccaaactgaaacagaatgaagtagctaatgtccagccttcttccaaaagagccagaattgaagacgtaccacctcccactaaaaagctaactccagaattgaccccttttgtgcttttcactggattcgagcctgtccaggttcaacagtatattaagaagctctacattcttggtggagaggttgcggagtctgcacagaagtgcacacacctcattgccagcaaagtgactcgcaccgtgaagttcctgacggcgatttctgtcgtgaagcacatagtgacgccagagtggctggaagaatgcttcaggtgtcagaagttcattgatgagcagaactacattctccgagatgctgaggcagaagtacttttctctttcagcttggaagaatccttaaaacgggcacacgtttctccactctttaaggcaaaatatttttacatcacacctggaatctgcccaagtctttccactatgaaggcaatcgtagagtgtgcaggaggaaaggtgttatccaagcagccatctttccggaagctcatggagcacaagcagaactcgagtttgtcggaaataattttaatatcctgtgaaaatgaccttcatttatgccgagaatattttgccagaggcatagatgttcacaatgcagagttcgttctgactggagtgctcactcaaacgctggactatgaatcatataagtttaactgatggcgtctaggctgccgtgcatgtcgactcctgcggtgcggggctggctgtctggctggcgaggagctgctgcgcttccttcacatgctcttgttttccagctgctttcctgggggatcagactgtgaagcaggaagacagatataataaatatactgcatctttttaagatgtgcaattttattctgaggaaacataaattatgttttgtattatatgactttaagagcccacattaggttttatgattcatttgccaggtttttaaatgttttcacaaaactgttacgggacttcaactagaaataaaatggtgtaaataaagaccttgctatctctaaattatggatgttaaagatttgaaatgttttgtactttgattatttttatttcttatactctgttttcttttatattgatatcttgcccacattttaaataaatgtacttttgaacttaaaaaaaaaaaaaaaaa

SEQ ID NO: 14 - DNA, 3723 bp, Homo sapiens
cggggccgggcgccgccgcggagcctcccgggccgccgcgatcatgtcggaccaggcgcccaaagttcctgaggagatgttcagggaggtcaagtattacgcggtgggcgacatcgacccgcaggttattcagcttctcaaggctggaaaagcgaaggaagtttcctacaatgcactagcctcacacataatctcagaggatggggacaatccagaggtgggagaagctcgggaagtctttgacttacctgttgtaaagccttcttgggtgattctgtccgttcagtgtggaactcttctgccagtaaatggtttttctccagaatcatgtcagattttttttggaatcactgcctgcctttctcaggtgtcatctgaagacagaagtgccctgtgggctttggttacgttctatgggggagattgccagctaaccctcaataagaaatgcacgcatttgattgttccagagccaaagggggagaaatacgaatgtgctttaaagcgagcaagtattaaaattgtgactcctgactgggttctggattgcgtatcagagaaaaccaaaaaggacgaagcattttatcatcctcgtctgattatttatgaagaggaagaagaggaagaggaagaggaggaggaagtagaaaatgaggaacaagattctcagaatgagggtagtacagatgagaagtcaagccctgccagctctcaagaagggtctccttcaggtgaccagcagttttcacctaaatccaacactgaaaaatctaaaggggaattaatgtttgatgattcttcagattcatcaccggaaaaacaggagagaaatttaaactggaccccggccgaagtcccacagttagctgcagcaaaacgcaggctgcctcagggaaaggagcctgggttgattaacttgtgtgccaatgtcccacccgtcccaggtaacattttgccccctgaggtccggggtaatttaatggctgctggacaaaacctccaaagttctgaaagatcagaaatgatagctacctggagtccagctgtacggacactgaggaatattactaataatgctgacattcagcagatgaaccggccatcaaatgtagcacatatcttacagactctttcagcacctacgaaaaatttagaacagcaggtgaatcacagccagcagggacatacaaatgccaatgcagtgctgtttagccaagtgaaagtgactccagagacacacatgctacagcagcagcagcaggcccagcagcagcagcagcagcacccggttttacaccttcagccccagcagataatgcagctccagcagcagcagcagcagcagatctctcagcaaccttacccccagcagccgccgcatccattttcacagcaacagcagcagcagcagcaagcccatccgcatcagttttcacagcaacagctacagtttccacagcaacagttgcatcctccacagcagctgcatcgccctcagcagcagctccagccctttcagcagcagcatgccctgcagcagcagttccatcagctgcagcagcaccagctccagcagcagcagcttgcccagctccagcagcagcacagcctgctccagcagcagcagcaacagcagattcagcagcagcagctccagcgcatgcaccagcagcagcagcagcagcagatgcaaagtcagacagcgccacacttgagtcagacgtcacaggcgctgcagcatcaggttccacctcagcagcccccgcagcagcagcagcaacagcagccaccaccatcgcctcagcagcatcagctttttggacatgatccagcagtggagattccagaagaaggcttcttattgggatgtgtgtttgcaattgcggattatccagagcagatgtctgataagcaactgctggccacctggaaaaggataatccaggcacatggcggcactgttgaccccaccttcacgagtcgatgcacgcaccttctctgtgagagtcaagtcagcagcgcgtatgcacaggcaataagagaaagaaagagatgtgttactgcacactggttaaacacagtcttaaagaagaagaaaatggtaccgccgcaccgagcccttcacttcccagtggccttcccaccaggaggaaagccatgttcacagcatattatttctgtgactggatttgttgatagtgacagagatgacctaaaattaatggcttatttggcaggtgccaaatatacgggttatctatgccgcagcaacacagtcctcatctgtaaagaaccaactggtttaaagtatgaaaaagccaaagagtggaggataccctgtgtcaacgcccagtggcttggcgacattcttctgggaaactttgaggcactgaggcagattcagtatagtcgctacacggcattcagtctgcaggatccatttgcccctacccagcatttagttttaaatcttttagatgcttggagagttcccttaaaagtgtctgcagagttgttgatgagtataagactacctcccaaactgaaacagaatgaagtagctaatgtccagccttcttccaaaagagccagaattgaagacgtaccacctcccactaaaaagctaactccagaattgaccccttttgtgcttttcactggattcgagcctgtccaggttcaacagtatattaagaagctctacattcttggtggagaggttgcggagtctgcacagaagtgcacacacctcattgccagcaaagtgactcgcaccgtgaagttcctgacggcgatttctgtcgtgaagcacatagtgacgccagagtggctggaagaatgcttcaggtgtcagaagttcattgatgagcagaactacattctccgagatgctgaggcagaagtacttttctctttcagcttggaagaatccttaaaacgggcacacgtttctccactctttaaggcaaaatatttttacatcacacctggaatctgcccaagtctttccactatgaaggcaatcgtagagtgtgcaggaggaaaggtgttatccaagcagccatctttccggaagctcatggagcacaagcagaactcgagtttgtcggaaataattttaatatcctgtgaaaatgaccttcatttatgccgagaatattttgccagaggcatagatgttcacaatgcagagttcgttctgactggagtgctcactcaaacgctggactatgaatcatataagtttaactgatggcgtctaggctgccgtgcatgtcgactcctgcggtgcggggctggctgtctggctggcgaggagctgctgcgcttccttcacatgctcttgttttccagctgctttcctgggggatcagactgtgaagcaggaagacagatataataaatatactgcatctttttaagatgtgcaattttattctgaggaaacataaattatgttttgtattatatgactttaagagcccacattaggttttatgattcatttgccaggtttttaaatgttttcacaaaactgttacgggacttcaactagaaataaaatggtgtaaataaagaccttgctatctctaaattatggatgttaaagatttgaaatgttttgtactttgattatttttatttcttatactctgttttcttttatattgatatcttgcccacattttaaataaatgtacttttgaacttaaaaaaaaaaaa

SEQ ID NO: 15 - protein, 757 aa, Homo sapiens
MAAGQNLQSSERSEMIATWSPAVRTLRNITNNADIQQMNRPSNVAHILQTLSAPTKNLEQQVNHSQQGHTNANAVLFSQVKVTPETHMLQQQQQAQQQQQQHPVLHLQPQQIMQLQQQQQQQISQQPYPQQPPHPFSQQQQQQQQAHPHQFSQQQLQFPQQQLHPPQQLHRPQQQLQPFQQQHALQQQFHQLQQHQLQQQQLAQLQQQHSLLQQQQQQQIQQQQLQRMHQQQQQQQMQSQTAPHLSQTSQALQHQVPPQQPPQQQQQQQPPPSPQQHQLFGHDPAVEIPEEGFLLGCVFAIADYPEQMSDKQLLATWKRIIQAHGGTVDPTFTSRCTHLLCESQVSSAYAQAIRERKRCVTAHWLNTVLKKKKMVPPHRALHFPVAFPPGGKPCSQHIISVTGFVDSDRDDLKLMAYLAGAKYTGYLCRSNTVLICKEPTGLKYEKAKEWRIPCVNAQWLGDILLGNFEALRQIQYSRYTAFSLQDPFAPTQHLVLNLLDAWRVPLKVSAELLMSIRLPPKLKQNEVANVQPSSKRARIEDVPPPTKKLTPELTPFVLFTGFEPVQVQQYIKKLYILGGEVAESAQKCTHLIASKVTRTVKFLTAISVVKHIVTPEWLEECFRCQKFIDEQNYILRDAEAEVLFSFSLEESLKRAHVSPLFKAKYFYITPGICPSLSTMKAIVECAGGKVLSKQPSFRKLMEHKQNSSLSEIILISCENDLHLCREYFARGIDVHNAEFVLTGVLTQTLDYESYKFN

SEQ ID NO: 16 - protein, 1069 aa, Homo sapiens
MSDQAPKVPEEMFREVKYYAVGDIDPQVIQLLKAGKAKEVSYNALASHIISEDGDNPEVGEAREVFDLPVVKPSWVILSVQCGTLLPVNGFSPESCQIFFGITACLSQVSSEDRSALWALVTFYGGDCQLTLNKKCTHLIVPEPKGEKYECALKRASIKIVTPDWVLDCVSEKTKKDEAFYHPRLIIYEEEEEEEEEEEEVENEEQDSQNEGSTDEKSSPASSQEGSPSGDQQFSPKSNTEKSKGELMFDDSSDSSPEKQERNLNWTPAEVPQLAAAKRRLPQGKEPGLINLCANVPPVPGNILPPEVRGNLMAAGQNLQSSERSEMIATWSPAVRTLRNITNNADIQQMNRPSNVAHILQTLSAPTKNLEQQVNHSQQGHTNANAVLFSQVKVTPETHMLQQQQQAQQQQQQHPVLHLQPQQIMQLQQQQQQQISQQPYPQQPPHPFSQQQQQQQQAHPHQFSQQQLQFPQQQLHPPQQLHRPQQQLQPFQQQHALQQQFHQLQQHQLQQQQLAQLQQQHSLLQQQQQQQIQQQQLQRMHQQQQQQQMQSQTAPHLSQTSQALQHQVPPQQPPQQQQQQQPPPSPQQHQLFGHDPAVEIPEEGFLLGCVFAIADYPEQMSDKQLLATWKRIIQAHGGTVDPTFTSRCTHLLCESQVSSAYAQAIRERKRCVTAHWLNTVLKKKKMVPPHRALHFPVAFPPGGKPCSQHIISVTGFVDSDRDDLKLMAYLAGAKYTGYLCRSNTVLICKEPTGLKYEKAKEWRIPCVNAQWLGDILLGNFEALRQIQYSRYTAFSLQDPFAPTQHLVLNLLDAWRVPLKVSAELLMSIRLPPKLKQNEVANVQPSSKRARIEDVPPPTKKLTPELTPFVLFTGFEPVQVQQYIKKLYILGGEVAESAQKCTHLIASKVTRTVKFLTAISVVKHIVTPEWLEECFRCQKFIDEQNYILRDAEAEVLFSFSLEESLKRAHVSPLFKAKYFYITPGICPSLSTMKAIVECAGGKVLSKQPSFRKLMEHKQNSSLSEIILISCENDLHLCREYFARGIDVHNAEFVLTGVLTQTLDYESYKFN

SEQ ID NO: 17 - DNA, 2137 bp, Homo sapiens
ctcgagagttgccggggaggagcggagcggtgcggaggctctgctcggatcgaggtctgcagcgcagcttcgggagcatgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaatagctgccctcccctccggactggtgccctcctcactcccacctgcatctggggcccatactggttggctcccgcggtgccatgtctgcagtgtgccccccagccccggtggctgggcagagctgcatcatccttgcaggtgggggttgctgtataagttatttttgtacatgttcgggtgtgggttctacagccttgtccccctccccctcaaccccaccatatgaattgtacagaatatttctatt

SEQ ID NO: 18 - DNA, 2204 bp, Homo sapiens
gagcggtgcggaggctctgctcggatcgaggtctgcagcgcagcttcgggagcatgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaatagctgccctcccctccggactggtgccctcctcactcccacctgcatctggggcccatactggttggctcccgcggtgccatgtctgcagtgtgccccccagccccggtggctgggcagagctgcatcatccttgcaggtgggggttgctgtgtaagttatttttgtacatgttcgggtgtgggttctacagccttgtccccctccccctcaaccccaccatatgaattgtacagaatatttctattgaattcggaactgtcctttccttggctttatgcacattaaacagatgtgaatattcaaaaaaaaaaaaaaaaaaaaaaaaaaaaaaaaaaa

SEQ ID NO: 19 - DNA, 2795 bp, Homo sapiens
gcacaagtggaccggggtgttgggtgctagtcggcaccagaggcaagggtgcgaggaccacggccggctcggacgtgtgaccgcgcctagggggtggcagcgggcagtgcggggcggcaaggcgaccatggagcttttgcggactatcacctaccagccagccgccagcaccaaaatgtgcgagcaggcgctgggcaagggttgcggagcggactcgaagaagaagcggccgccgcagccccccgaggaatcgcagccacctcagtcccaggcgcaagtgcccccggcggcccctcaccaccatcaccaccattcgcactcggggccggagatctcgcggattatcgtcgaccccacgactgggaagcgctactgccggggcaaagtgctgggaaagggtggctttgcaaaatgttacgagatgacagatttgacaaataacaaagtctacgccgcaaaaattattcctcacagcagagtagctaaacctcatcaaagggaaaagattgacaaagaaatagagcttcacagaattcttcatcataagcatgtagtgcagttttaccactacttcgaggacaaagaaaacatttacattctcttggaatactgcagtagaaggtcaatggctcatattttgaaagcaagaaaggtgttgacagagccagaagttcgatactacctcaggcagattgtgtctggactgaaataccttcatgaacaagaaatcttgcacagagatctcaaactagggaacttttttattaatgaagccatggaactaaaagttggggacttcggtctggcagccaggctagaacccttggaacacagaaggagaacgatatgtggtaccccaaattatctctctcctgaagtcctcaacaaacaaggacatggctgtgaatcagacatttgggccctgggctgtgtaatgtatacaatgttactagggaggcccccatttgaaactacaaatctcaaagaaacttataggtgcataagggaagcaaggtatacaatgccgtcctcattgctggctcctgccaagcacttaattgctagtatgttgtccaaaaacccagaggatcgtcccagtttggatgacatcattcgacatgacttttttttgcagggcttcactccggacagactgtcttctagctgttgtcatacagttccagatttccacttatcaagcccagctaagaatttctttaagaaagcagctgctgctctttttggtggcaaaaaagacaaagcaagatatattgacacacataatagagtgtctaaagaagatgaagacatctacaagcttaggcatgatttgaaaaagacttcaataactcagcaacccagcaaacacaggacagatgaggagctccagccacctaccaccacagttgccaggtctggaacacccgcagtagaaaacaagcagcagattggggatgctattcggatgatagtcagagggactcttggcagctgtagcagcagcagtgaatgccttgaagacagtaccatgggaagtgttgcagacacagtggcaagggttcttcggggatgtctggaaaacatgccggaagctgattgcattcccaaagagcagctgagcacatcatttcagtgggtcaccaaatgggttgattactctaacaaatatggctttgggtaccagctctcagaccacaccgtcggtgtccttttcaacaatggtgctcacatgagcctccttccagacaaaaaaacagttcactattacgcagagcttggccaatgctcagttttcccagcaacagatgctcctgagcaatttattagtcaagtgacggtgctgaaatacttttctcattacatggaggagaacctcatggatggtggagatctgcctagtgttactgatattcgaagacctcggctctacctccttcagtggctaaaatctgataaggccctaatgatgctctttaatgatggcacctttcaggtgaatttctaccatgatcatacaaaaatcatcatctgtagccaaaatgaagaataccttctcacctacatcaatgaggataggatatctacaactttcaggctgacaactctgctgatgtctggctgttcatcagaattaaaaaatcgaatggaatatgccctgaacatgctcttacaaagatgtaactgaaagacttttcgaatggaccctatgggactcctcttttccactgtgagatctacagggaagccaaaagaatgatctagagtatgttgaagaagatggacatgtggtggtacgaaaacaattcccctgtggcctgctggactggttggaaccagaacaggctaaggcatacagttcttgactttggacaatccaagagtgaaccagaatgcagttttccttgagatacctgttttaaaaggtttttcagacaattttgcagaaaggtgcattgattcttaaattctctctgttgagagcatttcagccagaggactttggaactgtgaatatacttcctgaaggggagggagaagggaggaagctcccatgttgtttaaaggctgtaattggagcagcttttggctgcgtaactgtgaactatggccatatataattttttttcattaatttttgaagatacttgtggctggaaaagtgcattccttgttaataaactttttatttattacagcccaaagagcagtatttattatcaaaatgtctttttttttatgttgaccattttaaaccgttggcaataaagagtatgaaaacgcagaaaaaaaaaaaa

SEQ ID NO: 20 - DNA, 2369 bp, Homo sapiens
cctgggcgccagcgcagcgtagcaaatccaggcagcgccacgcgcggccggggccgggcggaaccgagaagccgggaccgcgctgcgacgcgccggccgcatggagcctgccgccggtttcctgtctccgcgccccttccagcgtgcggccgccgcgcccgctcccccggccgggcccgggccgcctccgagtgccttgcgcggacctgagctggagatgctggccgggctaccgacgtcagaccccgggcgcctcatcacggacccgcgcagcggccgcacctacctcaaaggccgcttgttgggcaaggggggcttcgcccgctgctacgaggccactgacacagagactggcagcgcctacgctgtcaaagtcatcccgcagagccgcgtcgccaagccgcatcagcgcgagaagatcctaaatgagattgagctgcaccgagacctgcagcaccgccacatcgtgcgtttttcgcaccactttgaggacgctgacaacatctacattttcttggagctctgcagccgaaagtccctggcccacatctggaaggcccggcacaccctgttggagccagaagtgcgctactacctgcggcagatcctttctggcctcaagtacttgcaccagcgcggcatcttgcaccgggacctcaagttgggaaattttttcatcactgagaacatggaactgaaggtgggggattttgggctggcagcccggttggagcctccggagcagaggaagaagaccatctgtggcacccccaactatgtggctccagaagtgctgctgagacagggccacggccctgaggcggatgtatggtcactgggctgtgtcatgtacacgctgctctgcgggagccctccctttgagacggctgacctgaaggagacgtaccgctgcatcaagcaggttcactacacgctgcctgccagcctctcactgcctgcccggcagctcctggccgccatccttcgggcctcaccccgagaccgcccctctattgaccagatcctgcgccatgacttctttaccaagggctacacccccgatcgactccctatcagcagctgcgtgacagtcccagacctgacaccccccaacccagctaggagtctgtttgccaaagttaccaagagcctctttggcagaaagaagaagagtaagaatcatgcccaggagagggatgaggtctccggtttggtgagcggcctcatgcgcacatccgttggccatcaggatgccaggccagaggctccagcagcttctggcccagcccctgtcagcctggtagagacagcacctgaagacagctcaccccgtgggacactggcaagcagtggagatggatttgaagaaggtctgactgtggccacagtagtggagtcagccctttgtgctctgagaaattgtatagccttcatgcccccagcggaacagaacccggcccccctggcccagccagagcctctggtgtgggtcagcaagtgggttgactactccaataagttcggctttgggtatcaactgtccagccgccgtgtggctgtgctcttcaacgatggcacacatatggccctgtcggccaacagaaagactgtgcactacaatcccaccagcacaaagcacttctccttctccgtgggtgctgtgccccgggccctgcagcctcagctgggtatcctgcggtacttcgcctcctacatggagcagcacctcatgaagggtggagatctgcccagtgtggaagaggtagaggtacctgctccgcccttgctgctgcagtgggtcaagacggatcaggctctcctcatgctgtttagtgatggcactgtccaggtgaacttctacggggaccacaccaagctgattctcagtggctgggagcccctccttgtgacttttgtggcccgaaatcgtagtgcttgtacttacctcgcttcccaccttcggcagctgggctgctctccagacctgcggcagcgactccgctatgctctgcgcctgctccgggaccgcagcccagcctaggacccaagccctgaggcctgaggcctgtgcctgtcaggctctggcccttgcctttgtggccttcccccttcctttggtgcctcactgggggctttgggccgaatcccccagggaatcagggaccagctttactggagttgggggcggcttgtcttcgctggctcctaccccatctccaagataagcctgagccttagctcccagctagggggcgttatttatggaccacttttatttattgtcagacacttatttattgggatgtgagccccaggggggcctcctcctaggataataaacaattttgcagaattggaaaaaaaaaaaa

SEQ ID NO: 21 - DNA, 2381 bp, Xenopus laevis
gggccccagggactgagcagcagaaggagtagcagctacacggcttccctcagcctcggacaacctgccacgtcctgccccatcgatctcccctgatctgcccagcacttccccattcatcaggcgcctcaatactcgagctttaatagccggcaccccgcactatggctcaagtggccggtaagaaactgactgtggccccagaggccgctaaacccccaggaattcccgggagctcctcggccgtcaaagagatcccagagattctagtggatccccgaacccggaggcgatacctgagaggtcgattcctgggcaaaggtggattcgccaagtgctacgagatcaccgacctggagagccgggaggtatttgctgggaagattgtgcccaagaccatgttgctcaagccccaccagaaggataagatgaccatggagatcgccatccagcgcagcctggaccaccggcatgtcgtgggcttccatggcttctttgaggacaatgacttcgtgtatgtggtactggagctgtgcaggaggaggtctctgttggagctgcacaagaggagaaaagcggttacagagccagaagctcgctactatttgaaacagaccatttcgggatgtcagtatctccatagcaaccgagtcattcacagagacctcaagctcggaaacttgttccttaatgatgaaatggaggtcaaaataggtgactttgggctggcaaccaaagtggaatatgatggcgagcgcaaaaagaccctctgtggcactccaaactacattgcacctgaggtgttgggcaagaagggccacagttttgaagtggacatatggtcaataggatgcatcatgtacacactgctggtggggaaacctccctttgagacatcatgcctgaaagaaacctacatgagaattaaaaagaatgaatactccatccccaagcacattaaccctgtggcagcagcacttatacagaagatgctccgttctgacccaacctcaaggcccacaatagacgacttgctgaatgacgagttctttacttctggctacattccttcccggctccccacaacctgcttaactgtgcccccaaggttttccattgcgcccagcactattgatcaaagcttaaggaagccacttactgcaattaataaagggcaagactctccactggttgaaaagcaggtggctcctgcaaaggaagaggagatgcagcagccggagttcacggagcctgcagattgttacctatctgagatgctccagcagctgacatgtttgaatgcagtcaagccttctgagagagcgcttatccgccaagaggaagccgaggatccggcatccattcccatattctggatcagcaaatgggtggattactcggacaaatacggattaggatatcagctgtgtgataacagtgtaggggtgctcttcaatgactccacacggttgataatgtacaatgatggagacagcctgcagtacattgagcggaacaatacagaatcctacctcaacgtgcgctcctaccctactaccttaaccaaaaagatcacactgctgaagtacttcagaaactacatgagtgagcacctattgaaggccggtgccaacacgactcctcgggagggtgatgaactggctcgtctccccttcttgcgcacctggttccggacacgcagtgccattatccttcacctgagcaatggaactgttcagatcaacttcttccaggatcacaccaagataatcctgtgcccccttatggctgcggtgtcctacatagatgaaaagcgtgagttccgcacgtacaagctgagcctgattcaagaatttggctgctgcaaagagctcgcaagccgtctccggtacgcacgcacaatggtggagaaacttcagagctcaaagtcagccgttgcacacgtaaaggcctcggcatagccggccaagcaaactatggactccccagaaacaaacccatattcttgggtttctggaagcacaagaccttgtttcaagtcctaggagcacccgtctttttaattttaagccgaagctgacatgttctagggtgagatggttcgttaagcttgccaagactgtacagtactcgcgtgacgtttccataaaaatatatcttaagtgccactcgttgcgtctgggtaatcatggatatgtgatgtagatacctgctgggctctgtatgaacctgtgtcctcctgtttttctctgtcaacgttgtctggtttttaatgtaaaaatataatctttaatacttttgtatattatcagattaaagttctttgtatagccgtggaaaaaaaaaaaaaaaaaaaaaaaaaaaaaaaaaaaaaactcgag

SEQ ID NO: 22 - DNA, 2379 bp, Drosophila melanogaster
cgaaacgggcacaccaattcagctgacgcttcacgtccaattcattttggcatttaaaaaatcgaacggtttcggcgcgttctccgctttgtgcttggttttcgtgcattcgatgggcaaaaccgagatttgatcgccgcttctcttgtcaaccgtgtggatctgatctccgtttccgaggcactttcagccgatcgccggtggaattataggctcagaacgaggagtatcgccgcaaaactccaggcgaacgcaaacgcaaaaggggcagtccgtagtaaagaagaaaggagagcaagatggccgcgaagcccgaggataagagcacggatattccggatcgcctcgtcgacatcaaccagcggaaaacctacaagcgcatgcggttcttcggcaagggcgggtttgcaaaatgttacgagatcatcgatgtggaaaccgacgacgtcttcgccggcaagatcgtatccaagaagctgatgatcaaacacaatcagaaggagaagaccgcccaggagataactattcaccgcagccttaaccatccgaacattgtcaagtttcacaactactttgaagattcgcagaatatctacattgtgctggagctgtgcaagaaaagatccatgatggagctgcacaaacgtaggaaaagcattacggagttcgaatgccgctactacatttaccagataatccagggcgttaagtacttgcacgataaccgcattatccatcgagatctgaagctgggcaatctcttcctcaacgatttgttgcacgtgaagatcggggatttcgggttggccacgcgcattgagtatgagggcgagcgaaaaaagaccttatgcggaacgcccaactatatagccccggagatcctcaccaagaagggccactccttcgaggtggacatctggtcgattggctgcgtcatgtacacactgcttgtgggccagccgccgttcgaaaccaagactctgaaggatacgtactcgaaaatcaagaagtgcgagtaccgcgtgcccagctacttaaggaaaccggcggcggatatggtcatcgccatgctgcagccaaatccggagagccgcccggcaattggtcagctgctgaactttgagttcctcaagggctcaaaggtgcccatgttcttgcccagctcttgtctgacaatggcgccgcgtatcggcagcaacgacaccatcgaggattcgatgcaccgcaagccactgatggagatgaacggcatcaggcccgacgacactcgtctggagtcgaccttcctcaaggccaatctgcacgacgccattaccgcgtcagcgcaggtgtgccgccacagcgaggactatcgcagcgatatcgagagcctgtaccagcagctcactaatcttatcaacggaaagccgcgaattctgcaaggcaatctgggcgacgagaacacagatcctgcagcgcagccgctcttctggatatccaagtgggttgactacagcgacaagtacggatttggttaccagctgtgcgatgagggcatcggcgtgatgttcaacgacaccacaaagctgatcctgctgccgaatcagatcaacgtacacttcatcgacaaggatggcaaggagacgtacatgaccaccacggattactgcaagtcgcttgacaagaagatgaagctgctgtcgtactttaagcgctacatgatcgagcacctggtgaaggcaggtgccaacaatgtgaacattgagagcgatcaaatctcgcgtatgccccatttacactcctggttccgtacaacatgtgccgtagttatgcatttgaccaacggttctgtgcagctaaacttctcagatcacatgaagctcatcctctgcccgcgcatgagtgctataacctatatggaccaggagaagaacttccgcacctaccgattttcgaccattgtggagaacggcgtgtctaaagacttgtaccagaagatccgatatgcccaggagaaacttaggaaaatgctggagaagatgttcacataagcgtagccagcccaactatcattataaggccgaatgttagtttaacgtaattcacgaatgccctggccaacttcatttatagcccagaaagtatcctcctctcccatcatcttttaaaattgtagttcccgttcaaattgatttgttcgatgtttatagaatttatttgtttttgccccttccccttcatatcgaaaatactgcttaagttatattcatcgtcagtgttgggcctccctcaaaagtaatttaatatatctgtttaatggttttcgtacacgatccgatcacttaatgcattttaaagagatcaaattaaatgtttaaactaaaaaaaaaaaaaaaaaaa

SEQ ID NO: 23 - DNA, 2629 bp, Drosophila melanogaster
agccagcgaaacgggcacaccaattcagctgacgcttcacgtccaattcattttggcatttaaaaaatcgaacggtttcggcgcgttctccgctttgtgcttggttttcgtgcattcgatgggcaaaaccgagatttgatcgccgcttctcttgtcaaccgtgtggatctgatctccgtttccgaggcactttcagccgatcgccggtggaattataggctcagaacgaggagtatcgccgcaaaactccaggcgaacgcaaacgcaaaaggggcagtccgtagtaaagaagaaaggagagcaagatggccgcgaagcccgaggataagagcacggatattccggatcgcctcgtcgacatcaaccagcggaaaacctacaagcgcatgcggttcttcggcaagggcgggtttgcaaaatgttacgagatcatcgatgtggaaaccgacgacgtcttcgccggcaagatcgtatccaagaagctgatgatcaaacacaatcagaaggagaagaccgcccaggagataactattcaccgcagccttaaccatccgaacattgtcaagtttcacaactactttgaagattcgcagaatatctacattgtgctggagctgtgcaagaaaagatccatgatggagctgcacaaacgtaggaaaagcattacggagttcgaatgccgctactacatttaccagataatccagggcgttaagtacttgcacgataaccgcattatccatcgagatctgaagctgggcaatctcttcctcaacgatttgttgcacgtgaagatcggggatttcgggttggccacgcgcattgagtatgagggcgagcgaaaaaagaccttatgcggaacgcccaactatatagccccggagatcctcaccaagaagggccactccttcgaggtggacatctggtcgattggctgcgtcatgtacacactgcttgtgggccagccgccgttcgaaaccaagactctgaaggatacgtactcgaaaatcaagaagtgcgagtaccgcgtgcccagctacttaaggaaaccggcggcggatatggtcatcgccatgctgcagccaaatccggagagccgcccggcaattggtcagctgctgaactttgagttcctcaagggctcaaaggtgcccatgttcttgcccagctcttgtctgacaatggcgccgcgtatcggcagcaacgacaccatcgaggattcgatgcaccgcaagccactgatggagatgaacggcatcaggcccgacgacactcgtctggagtcgaccttcctcaaggccaatctgcacgacgccattaccgcgtcagcgcaggtgtgccgccacagcgaggactatcgcagcgatatcgagagcctgtaccagcagctcactaatcttatcaacggaaagccgcgaattctgcaaggcaatctgggcgacgagaacacagatcctgcagcgcagccgctcttctggatatccaagtgggttgactacagcgacaagtacggatttggttaccagctgtgcgatgagggcatcggcgtgatgttcaacgacaccacaaagctgatcctgctgccgaatcagatcaacgtacacttcatcgacaaggatggcaaggagacgtacatgaccaccacggattactgcaagtcgcttgacaagaagatgaagctgctgtcgtactttaagcgctacatgatcgagcacctggtgaaggcaggtgccaacaatgtgaacattgagagcgatcaaatctcgcgtatgccccatttacactcctggttccgtacaacatgtgccgtagttatgcatttgaccaacggttctgtgcagctaaacttctcagatcacatgaagctcatcctctgcccgcgcatgagtgctataacctatatggaccaggagaagaacttccgcacctaccgattttcgaccattgtggagaacggcgtgtctaaagacttgtaccagaagatccgatatgcccaggagaaacttaggaaaatgctggagaagatgttcacataagcgtagccagcccaactatcattataaggccgaatgttagtttaacgtaattcacgaatgccctggccaacttcatttatagcccagaaagtatcctcctctcccatcatcttttaaaattgtagttcccgttcaaattgatttgttcgatgtttatagaatttatttgtttttgccccttccccttcatatcgaaaatactgcttaagttatattcatcgtcagtgttgggcctccctcaaaagtaatttaatatatctgtttaatggttttcgtacacgatccgatcacttaatgcattttaaagagatcaaattaaatgtttaaactaagcaaacgtgtttcgaaatgcctatattcaccgaggtgactgataacaaaattttaatgctggatacattataaaagtaatagtgtaatattgtgcgttcgtagtgcgctatagcgccatttaaaataatacataagttacaatactgctgcaaagtgtttaagtgtacaagtatattcaactttggccagaaatatctgtagctataggatacaatatgtaaatgcttttgaactaaaagcgaatatatataaaatttaat

SEQ ID NO: 24 - protein, 603 aa, Homo sapiens; identical to SEQ ID NO: 1

SEQ ID NO: 25 - protein, 603 aa, Mus musculus
MNAAAKAGKLARAPADLGKGGVPGDAVPGAPVAAPLAKEIPEVLVDPRSRRQYVRGRFLGKGGFAKCFEISDADTKEVFAGKIVPKSLLLKPHQKEKMSMEISIHRSLAHQHVVGFHDFFEDSDFVFVVLELCRRRSLLELHKRRKALTEPEARYYLRQIVLGCQYLHRNQVIHRDLKLGNLFLNEDLEVKIGDFGLATKVEYEGERKKTLCGTPNYIAPEVLSKKGHSFEVDVWSIGCIMYTLLVGKPPFETSCLKETYLRIKKNEYSIPKHINPVAASLIQKMLQTDPTARPTIHELLNDEFFTSGYIPARLPITCLTIPPRFSIAPSSLDPSSRKPLKVLNKGVENPLPDRPREKEEPVVRETNEAIECHLSDLLQQLTSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLNYFRNYMSEHLLKAGANITPREGDELARLPYLRTWFRTRSAIILHLSNGTVQINFFQDHTKLILCPLMAAVTYINEKRDFQTYRLSLLEEYGCCKELASRLRYARTMVDKLLSSRSASNRLKAS

SEQ ID NO: 26 - protein, 15 aa, Artificial Sequence
  Description of Artificial Sequence: Synthetic peptide
  MOD_RES (3)..(6): Any amino acid except Cys
  MISC_FEATURE (7)..(7): Phosphorylation
  MOD_RES (9)..(12): Any amino acid except Cys
MAXXXXTPXXXXAKK

SEQ ID NO: 27 - protein, 15 aa, Artificial Sequence
  Description of Artificial Sequence: Synthetic peptide
  MOD_RES (3)..(6): Any amino acid except Cys
  MISC_FEATURE (7)..(7): Phosphorylation
  MOD_RES (9)..(12): Any amino acid except Cys
MAXXXXSPXXXXAKK

SEQ ID NO: 28 - protein, 15 aa, Artificial Sequence
  Description of Artificial Sequence: Synthetic peptide
  MOD_RES (3)..(6): Any amino acid except Cys
  MISC_FEATURE (8)..(8): Phosphorylation
  MOD_RES (9)..(12): Any amino acid except Cys
MAXXXXSTXXXXAKK

SEQ ID NO: 29 - protein, 15 aa, Artificial Sequence
  Description of Artificial Sequence: Synthetic peptide
  MOD_RES (3)..(6): Any amino acid except Cys
  MISC_FEATURE (8)..(8): Phosphorylation
  MOD_RES (9)..(12): Any amino acid except Cys
MAXXXXSSXXXXAKK

SEQ ID NO: 30 - protein, 20 aa, Artificial Sequence
  Description of Artificial Sequence: Synthetic peptide
  MOD_RES (1)..(1): Biotin-Aminohexanoic acid
  MOD_RES (3)..(3): Aminohexanoic acid
  MOD_RES (7)..(8): Any amino acid except Cys
  MOD_RES (9)..(9): A biased mixture of Pro, Leu, Ile, Val, Phe, Met or Trp
  MOD_RES (10)..(10): Any amino acid except Cys
  MISC_FEATURE (11)..(11): Phosphorylated
  MOD_RES (13)..(16): Any amino acid except Cys
XGXGGAXXXXTPXXXXAKKK

SEQ ID NO: 31 - protein, 20 aa, Artificial Sequence
  Description of Artificial Sequence: Synthetic peptide
  MOD_RES (1)..(1): Biotin-Aminohexanoic acid
  MOD_RES (3)..(3): Aminohexanoic acid
  MOD_RES (7)..(8): Any amino acid except for Cys
  MOD_RES (9)..(9): A biased mixture of Phe, Leu, Ile, Val, Phe, Met or Trp
  MOD_RES (10)..(10): Any amino acid except for Cys
  MOD_RES (13)..(16): Any amino acid except for Cys
XGXGGAXXXXTPXXXXAKKK

SEQ ID NO: 32 - protein, 20 aa, Artificial Sequence
  Description of Artificial Sequence: Synthetic peptide
  MOD_RES (1)..(1): Biotin-Aminohexanoic acid
  MOD_RES (3)..(3): Aminohexanoic acid
  MOD_RES (7)..(10): Any amino acid except Cys
  MISC_FEATURE (11)..(11): Phosphorylation
  MOD_RES (12)..(16): Any amino acid except Cys
XGXGGAXXXXTXXXXXAKKK

SEQ ID NO: 33 - protein, 20 aa, Artificial Sequence
  Description of Artificial Sequence: Synthetic peptide
  MOD_RES (1)..(1): Biotin-Aminohexanoic acid
  MOD_RES (3)..(3): Aminohexanoic acid
  MOD_RES (7)..(10): Any amino acid except Cys
  MOD_RES (12)..(16): Any amino acid except Cys
XGXGGAXXXXTXXXXXAKKK

SEQ ID NO: 34 - protein, 15 aa, Artificial Sequence
  Description of Artificial Sequence: Synthetic peptide
MAGPMQSTPLNGAKK

SEQ ID NO: 35 - protein, 20 aa, Artificial Sequence
  Description of Artificial Sequence: Synthetic peptide
  MOD_RES (1)..(1): Biotin-Aminohexanoic acid
  MOD_RES (3)..(3): Aminohexanoic acid
  MOD_RES (7)..(9): Any amino acid except Cys
  MOD_RES (10)..(10): A biased mixture of Ala, Ile, Leu, Met, Asn, Pro, Ser, Thr or Val
  MOD_RES (11)..(11): Phosphorylated Ser or phosphorylated Thr
  MOD_RES (13)..(13): A biased mixture of 25% Glu and 75% Any amino acid except Arg, Cys, His or Lys
  MOD_RES (14)..(16): Any amino acid except Cys
XGXGGAXXXXXQXXXXAKKK

SEQ ID NO: 36 - protein, 20 aa, Artificial Sequence
  Description of Artificial Sequence: Synthetic peptide
  MOD_RES (1)..(1): Biotin-Aminohexanoic acid
  MOD_RES (3)..(3): Aminohexanoic acid
  MOD_RES (7)..(10): Any amino acid except for Cys
  MOD_RES (11)..(11): Phosphorylated Ser
  MOD_RES (12)..(16): Any amino acid except for Cys
XGXGGAXXXXSXXXXXAKKK

SEQ ID NO: 37 - protein, 2089 aa, Homo sapiens
MEDTQAIDWDVEEEEETEQSSESLRCNVEPVGRLHIFSGAHGPEKDFPLHLGKNVVGRMPDCSVALPFPSISKQHAEIEILAWDKAPILRDCGSLNGTQILRPPKVLSPGVSHRLRDQELILFADLLCQYHRLDVSLPFVSRGPLTVEETPRVQGETQPQRLLLAEDSEEEVDFLSERRMVKKSRTTSSSVIVPESDEEGHSPVLGGLGPPFAFNLNSDTDVEEGQQPATEEASSAARRGATVEAKQSEAEVVTEIQLEKDQPLVKERDNDTKVKRGAGNGVVPAGVILERSQPPGEDSDTDVDDDSRPPGRPAEVHLERAQPFGFIDSDTDAEEERIPATPVVIPMKKRKIFHGVGTRGPGAPGLAHLQESQAGSDTDVEEGKAPQAVPLEKSQASMVINSDTDDEEEVSAALTLAHLKESQPAIWNRDAEEDMPQRVVLLQRSQTTTERDSDTDVEEEELPVENREAVLKDHTKIRALVRAHSEKDQPPFGDSDDSVEADKSSPGIHLERSQASTTVDINTQVEKEVPPGSAIMHIKKHQVSVEGTNQTDVKAVGGPAKLLVVSLEEAWPLHGDCETDAEEGTSLTASVVADVRKSQLPAEGDAGAEWAAAVLKQERAHEVGAQGGPPVAQVEQDLPISRENLTDLVVDTDTLGESTQPQREGAQVPTGREREQHVGGTKDSEDNYGDSEDLDLQATQCFLENQGLEAVQSMEDEPTQAFMLTPPQELGPSHCSFQTTGTLDEPWEVLATQPFCLRESEDSETQPFDTHLEAYGPCLSPPRAIPGDQHPESPVHTEPMGIQGRGRQTVDKVMGIPKETAERVGPERGPLERETEKLLPERQTDVTGEEELTKGKQDREQKQLLARDTQRQESDKNGESASPERDRESLKVEIETSEEIQEKQVQKQTLPSKAFEREVERPVANRECDPAELEEKVPKVILERDTQRGEPEGGSQDQKGQASSPTPEPGVGAGDLPGPTSAPVPSGSQSGGRGSPVSPRRHQKGLLNCKMPPAEKASRIRAAEKVSRGDQESPDACLPPAVPEAPAPPQKPLNSQSQKHLAPPPLLSPLLPSIKPTVRKTRQDGSQEAPEAPLSSELEPFHPKPKIRTRKSSRMTPFPATSAAPEPHPSTSTAQPVTPKPTSQATRSRTNRSSVKTPEPVVPTAPELQPSTSTDQPVTSEPTSQVTRGRKSRSSVKTPETVVPTALELQPSTSTDRPVTSEPTSQATRGRKNRSSVKTPEPVVPTAPELQPSTSTDQPVTSEPTYQATRGRKNRSSVKTPEPVVPTAPELRPSTSTDRPVTPKPTSRTTRSRTNMSSVKTPETVVPTAPELQISTSTDQPVTPKPTSRTTRSRTNMSSVKNPESTVPIAPELPPSTSTEQPVTPEPTSRATRGRKNRSSGKTPETLVPTAPKLEPSTSTDQPVTPEPTSQATRGRTNRSSVKTPETVVPTAPELQPSTSTDQPVTPEPTSQATRGRTDRSSVKTPETVVPTAPELQASASTDQPVTSEPTSRTTRGRKNRSSVKTPETVVPAAPELQPPTSTDRPVTPEPTSRATRGRTNRSSVKTPESIVPIAPELQPSTSRNQLVTPEPTSRATRCRTNRSSVKTPEPVVPTAPEPHPTTSTDQPVTPKLTSRATRRKTNRSSVKTPKPVEPAASDLEPFTPTDQSVTPEAIAQGGQSKTLRSSTVRAMPVPTTPEFQSPVTTDQPISPEPITQPSCIKRQRAAGNPGSLAAPIDHKPCSAPLEPKSQASRNQRWGAVRAAESLTAIPEPASPQLLETPIHASQIQKVEPAGRSRFTPELQPKASQSRKRSLATMDSPPHQKQPQRGEVSQKTVIIKEEEEDTAEKPGKEEDVVTPKPGKRKRDQAEEEPNRIPSRSLRRTKLNQESTAPKVLFTGVVDARGERAVLALGGSLAGSAAEASHLVTDRIRRTVKFLCALGRGIPILSLDWLHQSRKAGFFLPPDEYVVTDPEQEKNFGFSLQDALSRARERRLLEGYEIYVTPGVQPPPPQMGEIISCCGGTYLPSMPRSYKPQRVVITCPQDFPHCSIPLRVGLPLLSPEFLLTGVLKQEAKPEAFVLSPLEMSST

SEQ ID NO: 38 - protein, 1972 aa, Homo sapiens
MDPTGSQLDSDFSQQDTPCLIIEDSQPESQVLEDDSGSHFSMLSRHLPNLQTHKENPVLDVVSNPEQTAGEERGDGNSGFNEHLKENKVADPVDSSNLDTCGSISQVIEQLPQPNRTSSVLGMSVESAPAVEEEKGEELEQKEKEKEEDTSGNTTHSLGAEDTASSQLGFGVLELSQSQDVEENTVPYEVDKEQLQSVTTNSGYTRLSDVDANTAIKHEEQSNEDIPIAEQSSKDIPVTAQPSKDVHVVKEQNPPPARSEDMPFSPKASVAAMEAKEQLSAQELMESGLQIQKSPEPEVLSTQEDLFDQSNKTVSSDGCSTPSREEGGCSLASTPATTLHLLQLSGQRSLVQDSLSTNSSDLVAPSPDAFRSTPFIVPSSPTEQEGRQDKPMDTSVLSEEGGEPFQKKLQSGEPVELENPPLLPESTVSPQASTPISQSTPVFPPGSLPIPSQPQFSHDIFIPSPSLEEQSNDGKKDGDMHSSSLTVECSKTSEIEPKNSPEDLGLSLTGDSCKLMLSTSEYSQSPKMESLSSHRIDEDGENTQIEDTEPMSPVLNSKFVPAENDSILMNPAQDGEVQLSQNDDKTKGDDTDTRDDISILATGCKGREETVAEDVCIDLTCDSGSQAVPSPATRSEALSSVLDQEEAMEIKEHHPEEGSSGSEVEEIPETPCESQGEELKEENMESVPLHLSLTETQSQGLCLQKEMPKKECSEAMEVETSVISIDSPQKLAILDQELEHKEQEAWEEATSEDSSVVIVDVKEPSPRVDVSCEPLEGVEKCSDSQSWEDIAPEIEPCAENRLDTKEEKSVEYEGDLKSGTAETEPVEQDSSQPSLPLVRADDPLRLDQELQQPQTQEKTSNSLTEDSKMANAKQLSSDAEAQKLGKPSAHASQSFCESSSETPFHFTLPKEGDIIPPLTGATPPLIGHLKLEPKRHSTPIGISNYPESTIATSDVMSESMVETHDPILGSGKGDSGAAPDVDDKLCLRMKLVSPETEASEESLQFNLEKPATGERKNGSTAVAESVASPQKTMSVLSCICEARQENEARSEDPPTTPIRGNLLHFPSSQGEEEKEKLEGDHTIRQSQQPMKPISPVKDPVSPASQKMVIQGPSSPQGEAMVTDVLEDQKEGRSTNKENPSKALIERPSQNNIGIQTMECSLRVPETVSAATQTIKNVCEQGTSTVDQNFGKQDATVQTERGSGEKPVSAPGDDTESLHSQGEEEFDMPQPPHGHVLHRHMRTIREVRTLVTRVITDVYYVDGTEVERKVTEETEEPIVECQECETEVSPSQTGGSSGDLGDISSFSSKASSLHRTSSGTSLSAMHSSGSSGKGAGPLRGKTSGTEPADFALPSSRGGPGKLSPRKGVSQTGTPVCEEDGDAGLGIRQGGKAPVTPRGRGRRGRPPSRTTGTRETAVPGPLGIEDISPNLSPDDKSFSRVVPRVPDSTRRTDVGAGALRRSDSPEIPFQAAAGPSDGLDASSPGNSFVGLRVVAKWSSNGYFYSGKITRDVGAGKYKLLFDDGYECDVLGKDILLCDPIPLDTEVTALSEDEYFSAGVVKGHRKESGELYYSIEKEGQRKWYKRMAVILSLEQGNRLREQYGLGPYEAVTPLTKAADISLDNLVEGKRKRRSNVSSPATPTASSSSSTTPTRKITESPRASMGVLSGKRKLITSEEERSPAKRGRKSATVKPGAVGAGEFVSPCESGDNTGEPSALEEQRGPLPLNKTLFLGYAFLLTMATTSDKLASRSKLPDGPTGSSEEEEEFLEIPPFNKQYTESQLRAGAGYILEDFNEAQCNTAYQCLLIADQHCRTRKYFLCLASGIPCVSHVWVHDSCHANQLQNYRNYLLPAGYSLEEQRILDWQPRENPFQNLKVLLVSDQQQNFLELWSEILMTGGAASVKQHHSSAHNKDIALGVFDVVVTDPSCPASVLKCAEALQLPVVSQEWVIQCLIVGERIGFKQHPKYKHDYVSH

SEQ ID NO: 39 - protein, 1309 aa, Saccharomyces cerevisiae
MSGQLVQWKSSPDRVTQSAIKEALHSPLADGDMNEMNVPVDPLENKVNSTNIIEGSPKANPNPVKFMNTSEIFQKSLGLLDESPRHDDELNIEVGDNDRPNANILHNERTPDLDRIANFFKSNRTPGKENLLTKYQSSDLEDTPLMLRKKMTFQTPTDPLEQKTFKKLKSDTGFCYYGEQNDGEENASLEVTEADATFVQMAERSADNYDCALEGIVTPKRYKDELSKSGGMQDERVQKTQIMISAESPNSISSYDKNKITGNGRTTRNVNKVFNNNEDNIGAIEEKNPVKKKSENYSSDDLRERNNQIIQSNESEEINELEKNLNVSGRENDVNNLDIDINSAVSGTPSRNNAEEEMYSSESVNNREPSKKWIFRYSKDKTENNSNRSTQIVNNPRTQEMPLDSISIDTQPLSKSFNTETNNELETQIIVSSLSQGISAQKGPVFHSTGQTEEIKTQIINSPEQNALNATFETPVTLSRINFEPILEVPETSSPSKNTMSKPSNSSPIPKEKDTFNIHEREVETNNVFSNDIQNSSNAATRDDIIIAGSSDFNEQKEITDRIYLQLSGKQISDSGSDETERMSPNELDTKKESTIMSEVELTQELPEVEEQQDLQTSPKKLVVEEETLMEIKKSKGNSLQLHDDNKECNSDKQDGTESLDVALIEHESKGQSSELQKNLMQLFPSESQEIIQNRRTIKRRQKDTIEIGEEEENRSTKTSPTKHLKRNSDLDAASIKREPSCSITIQTGETGSGKDSKEQSYVFPEGIRTADNSFLSKDDIIFGNAVWCQYTWNYKFYPGILLEVDTNQDGCWIYFETGRSLTKDEDIYYLDIRIGDAVTFDGNEYVVVGLECRSHDLNIIRCIRGYDTVHLKKKNASGLLGKRTLIKALSSISLDLSEWAKRAKIILEDNEKNKGDAYRYLRHPIRGRKSMTNVLSPKKHTDDEKDINTHTEVYNNEIESSSEKKEIVKKDSRDALAEHAGAPSLLFSSGEIRTGNVFDKCIFVLTSLFENREELRQTIESQGGTVIESGFSTLFNFTHPLAKSLVNKGNTDNIRELALKLAWKPHSLFADCRFACLITKRHLRSLKYLETLALGWPTLHWKFISACIEKKRIVPHLIYQYLLPSGESFRLSLDSPSKGGIIKSNNIFSFYTQFLRGSNLRDQICGVKKMLNDYIVIVWGRSELDSFVKFAFACLSAGRMLTIDLPNIDVDDTEPLLNALDSLVPRIGSELSNRKLKFLIYANENNGKSQMKLLERLRSQISLKFKKFNYIFHTESKEWLIQTIINEDTGFHDDITDNDIYNTISEVR

SEQ ID NO: 40 - DNA, 6270 bp, Homo sapiens
atggaggacacccaggctattgactgggatgttgaagaagaggaggagacagagcaatccagtgaatccttgaggtgtaacgtggagccagtagggcggctacatatctttagtggtgcccatggaccagaaaaagatttcccactacacctcgggaagaatgtggtaggccgaatgcctgactgctctgtggccctgccctttccatctatctccaaacaacatgcagagattgaaatcttagcctgggacaaggcacctatcctccgagactgtgggagccttaatggtactcaaatcctgagacctcctaaggttttgagccctggggtgagtcaccgtctgagggaccaggaattgattctctttgctgacttgctctgccagtaccatcgcctggatgtctctctgccctttgtctcccggggccctctgacagtagaagagacacccagagtacagggagaaactcaaccccagaggcttctgttggctgaggactcggaggaggaagtagattttctttctgaaaggcgtatggtaaaaaaatcaaggaccacatcttcctctgtgatagttccagagagtgatgaagaggggcattccccggtcctgggcggccttgggccgccttttgccttcaatttgaacagtgacacagatgtggaagaaggtcagcaaccagccacagaggaggcctcctcagctgccagaagaggtgccactgtagaggcaaagcagtctgaagctgaagttgtaactgaaatccagcttgaaaaggatcagcctttagtgaaggagagggacaatgatacaaaagtcaagaggggtgcagggaatggggtggttccagctggggtgattctggagaggagccaacctcctggagaggacagtgacacagatgtggatgatgacagcaggcctcctggaaggccagctgaggtccatttggaaagggctcagccttttggcttcatcgacagcgacactgatgcggaagaagagaggatcccagcaaccccagttgtcattcctatgaagaagaggaagatcttccatggagtaggtacaaggggtcctggagcaccaggcctggcccatctgcaggagagccaggctggtagtgatacagatgtggaagaaggcaaggccccacaggctgtccctctggagaaaagccaagcttccatggttatcaacagcgatacagatgacgaggaagaagtctcagcagcgctgactttggcacatctgaaagagagccagcctgctatatggaacagagatgcagaagaggacatgccccaacgtgtggtccttctgcagcgaagccaaaccaccactgagagagacagtgacacagacgtggaggaggaagagctcccagtggaaaatagagaagctgtcctcaaggatcacacaaagattagagcccttgttagagcacattcagaaaaggaccaacctccttttggggacagtgatgacagtgtggaagcagataagagctcacctgggatccacctggagagaagccaagcctccaccacagtggacatcaacacacaagtggagaaggaagtcccgccagggtcagccattatgcatataaagaagcatcaggtgtctgtggaggggacaaatcaaacagatgtgaaagcagttgggggaccagcaaagctgcttgtggtatctctagaggaagcctggcctctgcatggggactgtgaaacagatgcagaggagggcacctccctaacagcctcagtagttgcagatgtaagaaagagccagcttccagcagaaggggatgctggggcagagtgggctgcagctgttcttaagcaggagagagctcatgaggtgggggcccagggtgggccacctgtggcacaagtggagcaggacctccctatctcaagagagaacctcacagatctggtggtggacacagacactctaggggaatccacccagccacagagagagggagcccaggtccccacaggaagggagagagaacaacatgtgggtgggaccaaggactctgaagacaactatggtgattctgaagatctggacctacaagctacccagtgctttctggagaatcagggcctggaagcagtccagagcatggaggatgaacctacccaggccttcatgttgactccaccccaagagcttggcccttcccattgcagcttccagacaacaggtaccctagatgaaccatgggaggtcctggctacacagccattctgtctgagagagtctgaggactctgagacccagccttttgacacgcaccttgaggcctatggaccttgcctgtctccacctagggcaataccaggagaccaacatccagagagcccagttcacacagagccaatggggattcaaggcagagggaggcagactgtggataaagtcatgggtataccaaaagaaacagcagagagggtgggccctgagagagggccattggagagagaaactgagaaactgctaccagaaagacagacagatgtgacaggagaggaagaattaaccaaggggaaacaggacagagaacaaaaacagttgttagctagagacacccagagacaagaatctgacaaaaatggggaaagtgcaagtcctgaaagagatagggagagtttgaaggtagaaattgagacatctgaggaaatacaagagaaacaagtacagaagcagacccttccaagcaaagcatttgagagagaagtagagagaccagtagcaaacagagagtgcgatccagccgagttagaagagaaggtgcccaaagtgatcctggagagagatacacagagaggggagccagagggagggagccaggaccagaaagggcaggcctccagcccaacaccagagcctggggtgggggcgggggaccttccgggacctacctcagcccccgtaccttctgggagccagtcaggtggaaggggatccccagtgagccccaggaggcatcagaaaggcctcctgaattgcaagatgccacctgctgagaaggcttccaggatcagagctgctgagaaggtttccaggggcgatcaggaatctccagatgcttgtctgcctcctgcagtacctgaagccccagccccaccccaaaagccccttaactctcagagccagaaacatcttgcacctccgccccttctttctccccttttaccttctatcaagccaaccgttcgtaagaccaggcaagatgggagtcaggaagctccagaggctcccttgtcctcagagctggagcctttccacccaaagcctaaaattagaactcggaagtcctccagaatgacaccctttccagctacctctgctgcccctgagccccacccttccacctccacagcccagccagtcactcccaagcccacatctcaggccactaggagcaggacaaataggtcctctgtcaagacccctgaaccagttgtccccacagcccctgagctccagccttccacctccacagaccagcctgtcacctctgagcccacatctcaggttactaggggaagaaaaagtagatcctctgtcaagacccctgaaacagttgtgcccacagcccttgagctccagccttccacctccaccgaccgacctgtcacctctgaacccacctctcaggctactaggggaagaaaaaatagatcctctgtcaagacccctgaaccagttgtccccacagcccctgagctccagccttccacctccacagaccagcctgtcacttctgagcccacatatcaggctactaggggaagaaaaaatagatcctctgtcaagacccctgaaccagttgtgcccacagcccctgagctccggccttccacctccacagaccgacctgtcacccccaagcccacatctcggaccactaggagcaggacaaatatgtcctctgtcaagacccctgaaacagttgtccccacagcccctgagctccagatttccacctccacagaccaacctgtcacccctaagcccacatctcggaccactaggagcaggacaaatatgtcctctgtgaagaaccctgaatcaactgtccctatagcccctgagctcccaccttccacctccacagagcagcctgtcacccctgagcccacatctcgggctactaggggaagaaaaaatagatcctctggcaagacccctgaaacacttgtccccacagcccctaagctcgagccttccacttccacagaccaacctgtcactcctgagcccacatctcaggccaccaggggcaggacaaataggtcctctgtgaagacccctgaaacagttgtccccacagcccctgagctccagccttccacctccacagaccagcctgttacccctgagcctacgtctcaggctactaggggaagaacagatagatcctctgtcaagactcctgaaacagttgtccccacagcccctgagctacaggcttccgcctccacagaccagcctgtcacctctgagcccacatctcggaccactaggggaagaaaaaatcggtcctctgtcaagacccctgaaacagttgtgcccgcagcccctgagctccagcctcccacctccacagaccgacctgtcacccctgagcccacatctcgggccactaggggcaggacaaataggtcctctgtcaagacccctgaatcaattgtccctatagcccctgagcttcagccttccacctccagaaaccagcttgtcacccctgagcccacatctcgggccactaggtgcaggacaaataggtcctctgtcaagacccctgagccagttgtccccacagcccctgagccccatcctaccacctccacagaccagcctgtcacccccaagctcacatctagggccactaggagaaagacaaataggtcctctgtcaagactcccaaaccagttgaaccagcagcctctgatcttgagccttttacccccacagaccagtccgtcacccctgaggccatagctcagggtggtcagagcaaaacactgaggtcttccacagtaagagctatgccggttcctaccacccctgaattccaatctcctgtcaccacagaccagcctatttcccctgagcctattactcaacccagttgcatcaagaggcagagagccgctgggaaccctggctccctcgcagctcccattgaccataagccttgctctgcacccttggaacctaaatcccaggcctcaaggaaccaaagatggggagcagtgagagcagctgaatcccttacagccattcctgagcctgcctctccccagcttcttgagacaccaattcatgcctcccagatccaaaaggtggaaccagcaggtagatctaggttcaccccggagctccagcctaaggcctctcaaagccgcaagaggtctttagctaccatggattcaccaccacatcaaaaacagccccaaagaggggaagtctcccagaagacagtgattatcaaggaagaggaagaagatactgcagagaagccagggaaggaagaggatgtcgtgactccaaaaccaggcaagagaaagagagaccaggcagaggaggagcccaacagaataccaagccgcagcctccgacggaccaaacttaaccaagaatcaacagcccccaaagtgctcttcacaggagtggtggatgctcggggagagcgggctgtgctggcactggggggaagtctggctggttcagcggcagaggcttcccacctggtcactgatcgcatccgccggacagtcaagttcctgtgtgccctggggcggggaatccccattctgtccctggactggctgcatcagtcccgcaaggctggtttcttcttacccccggatgaatatgtggtgaccgaccctgagcaagagaagaactttggctttagccttcaagacgcactgagcagggctcgggagcgaaggctgctagagggctatgagatctatgtgacccctggagtccagccaccaccacctcagatgggagagattattagctgctgtggaggcacatacctacccagcatgcctcggtcctataagcctcagagagttgtgatcacatgccctcaggacttccctcattgctccattccactacgggttgggctgcccctcctctcgcctgagttcctgctgactggagtgctgaagcaggaagccaagccagaggcctttgtcctctcccctttggagatgtcatccacctga

SEQ ID NO: 41 - DNA, 5919 bp, Homo sapiens
atggaccctactggaagtcagttggattcagatttctctcagcaagatactccttgcctgataattgaagattctcagcctgaaagccaggttctagaggatgattctggttctcacttcagtatgctatctcgacaccttcctaatctccagacgcacaaagaaaatcctgtgttggatgttgtgtccaatcctgaacaaacagctggagaagaacgaggagacggtaatagtgggttcaatgaacatttgaaagaaaacaaggttgcagaccctgtggattcttctaacttggacacatgtggttccatcagtcaggtcattgagcagttacctcagccaaacaggacaagcagtgttctgggaatgtcagtggaatctgctcctgctgtggaggaagagaagggagaagagttggaacagaaggagaaagagaaggaagaagatacttcaggcaatactacacattcccttggtgctgaagatactgcctcatcacagttgggttttggggttctggaactctcccagagccaggatgttgaggaaaatactgtgccatatgaagtggacaaagagcagctacaatcagtaaccaccaactctggttataccaggctgtctgatgtggatgctaatactgcaattaagcatgaagaacagtccaacgaagatatccccatagcagaacagtccagcaaggacatccctgtgacagcacagcccagtaaggatgtacatgttgtaaaagagcaaaatccaccacctgcaaggtcagaggacatgccttttagccccaaagcatctgttgctgctatggaagcaaaagaacagttgtctgcacaagaacttatggaaagtggactgcagattcagaagtcaccagagcctgaggttttgtcaactcaggaagacttgtttgaccagagcaataaaacagtatcttctgatggttgctctactccttcaagggaggaaggtgggtgttctttggcttccactcctgccaccactctgcatctcctgcagctctctggtcagaggtcccttgttcaggacagtctttccacgaattcttcagatcttgttgctccttctcctgatgctttccgatctactccttttatcgttcctagcagtcccacagagcaagaagggagacaagataagccaatggacacgtcagtgttatctgaagaaggaggagagccttttcagaagaaacttcaaagtggtgaaccagtggagttagaaaacccccctctcctgcctgagtccactgtatcaccacaagcctcaacaccaatatctcagagcacaccagtcttccctcctgggtcacttcctatcccatcccagcctcagttttctcatgacatttttattccttccccaagtctggaagaacaatcaaatgatgggaagaaagatggagatatgcatagttcatctttgacagttgagtgttctaaaacttcagagattgaaccaaagaattcccctgaggatcttgggctatctttgacaggggattcttgcaagttgatgctttctacaagtgaatatagtcagtccccaaagatggagagcttgagttctcacagaattgatgaagatggagaaaacacacagattgaggatacggaacccatgtctccagttctcaattctaaatttgttcctgctgaaaatgatagtatcctgatgaatccagcacaggatggtgaagtacaactgagtcagaatgatgacaaaacaaagggagatgatacagacaccagggatgacattagtattttagccactggttgcaagggcagagaagaaacggtagcagaagatgtttgtattgatctcacttgtgattcggggagtcaggcagttccgtcaccagctactcgatctgaggcactttctagtgtgttagatcaggaggaagctatggaaattaaagaacaccatccagaggaggggtcttcagggtctgaggtggaagaaatccctgagacaccttgtgaaagtcaaggagaggaactcaaagaagaaaatatggagagtgttccgttgcacctttctctgactgaaactcagtcccaagggttgtgtcttcaaaaggaaatgccaaaaaaagaatgctcagaagctatggaagttgaaaccagtgtgattagtattgattcccctcaaaagttggcaatacttgaccaagaattggaacataaggaacaggaagcttgggaagaagctacttcagaggactccagtgttgtcattgtagatgtgaaagagccatctcccagagttgatgtttcttgtgaacctttggagggagtggagaagtgctcagattcccagtcatgggaggatattgctccagaaatagaaccatgtgctgagaatagattagacaccaaggaagaaaagagtgtagaatatgaaggagatctgaaatcagggactgcagaaacagaacctgtagagcaagattcttcacagccttccttacctttagtgagagcagatgatcctttaagacttgaccaggagttgcagcagccccaaactcaggagaaaacaagtaattcattaacagaagactcaaaaatggctaatgcaaagcagctaagctcagatgcagaggcccagaagctggggaagccctctgcccatgcctcacaaagcttctgtgaaagttctagtgaaaccccatttcatttcactttgcctaaagaaggtgatatcatcccaccattgactggtgcaaccccacctcttattgggcacctaaaattggagcccaagagacacagtactcctattggtattagcaactatccagaaagcaccatagcaaccagtgatgtcatgtctgaaagcatggtggagacccatgatcccatacttgggagtggaaaaggggattctggggctgccccagacgtggatgataaattatgtctaagaatgaaactggttagtcctgagactgaggcgagtgaagagtctttgcagttcaacctggaaaagcctgcaactggtgaaagaaaaaatggatctactgctgttgctgagtctgttgccagtccccagaagaccatgtctgtgttgagctgtatctgtgaagccaggcaagagaatgaggctcgaagtgaggatccccccaccacacccatcagggggaacttgctccactttccaagttctcaaggagaagaggagaaagaaaaattggagggtgaccatacaatcaggcagagtcaacagcctatgaagcccattagtcctgtcaaggaccctgtttctcctgcttcccagaagatggtcatacaagggccatccagtcctcaaggagaggcaatggtgacagatgtgctagaagaccagaaagaaggacggagtactaataaggaaaatcctagtaaggccttgattgaaaggcccagccaaaataacataggaatccaaaccatggagtgttccttgagggtcccagaaactgtttcagcagcaacccagactataaagaatgtgtgtgagcaggggaccagtacagtggaccagaactttggaaagcaagatgccacagttcagactgagagggggagtggtgagaaaccagtcagtgctcctggggatgatacagagtcgctccatagccagggagaagaagagtttgatatgcctcagcctccacatggccatgtcttacatcgtcacatgagaacaatccgggaagtacgcacacttgtcactcgtgtcattacagatgtgtattatgtggatggaacagaagtagaaagaaaagtaactgaggagactgaagagccaattgtagagtgtcaggagtgtgaaactgaagtttccccttcacagactgggggctcctcaggtgacctgggggatatcagctccttctcctccaaggcatccagcttacaccgcacatcaagtgggacaagtctctcagctatgcacagcagtggaagctcagggaaaggagccggaccactcagagggaaaaccagcgggacagaacccgcagattttgccttacccagctcccgaggaggcccaggaaaactgagtcctagaaaaggggtcagtcagacagggacgccagtgtgtgaggaggatggtgatgcaggccttggcatcagacagggagggaaggctccagtcacgcctcgtgggcgtgggcgaaggggccgcccaccttctcggaccactggaaccagagaaacagctgtgcctggccccttgggcatagaggacatttcacctaacttgtcaccagatgataaatccttcagccgtgtcgtgccccgagtgccagactccaccagacgaacagatgtgggtgctggtgctttgcgtcgtagtgactctccagaaattcctttccaggctgctgctggcccttctgatggcttagatgcctcctctccaggaaatagctttgtagggctccgtgttgtagccaagtggtcatccaatggctacttttactctgggaaaatcacacgagatgtcggagctgggaagtataaattgctctttgatgatgggtacgaatgtgatgtgttgggcaaagacattctgttatgtgaccccatcccgctggacactgaagtgacggccctctcggaggatgagtatttcagtgcaggagtggtgaaaggacataggaaggagtctggggaactgtactacagcattgaaaaagaaggccaaagaaagtggtataagcgaatggctgtcatcctgtccttggagcaaggaaacagactgagagagcagtatgggcttggcccctatgaagcagtaacacctcttacaaaggcagcagatatcagcttagacaatttggtggaagggaagcggaaacggcgcagtaacgtcagctccccagccacccctactgcctccagtagcagcagcacaacccctacccgaaagatcacagaaagtcctcgtgcctccatgggagttctctcaggcaaaagaaaacttatcacttctgaagaggaacggtcccctgccaagcgaggtcgcaagtctgccacagtaaaacctggtgcagtaggggcaggagagtttgtgagcccctgtgagagtggagacaacaccggtgaaccctctgccctggaagagcagagagggcctttgcctctcaacaagaccttgtttctgggctacgcatttctccttaccatggccacaaccagtgacaagttggccagccgctccaaactgccagatggtcctacaggaagcagtgaagaagaggaggaatttttggaaattcctcctttcaacaagcagtatacagaatcccagcttcgagcaggagctggctatatccttgaagatttcaatgaagcccagtgtaacacagcttaccagtgtcttctaattgcggatcagcattgtcgaacccggaagtacttcctgtgccttgccagtgggattccttgtgtgtctcatgtctgggtccatgatagttgccatgccaaccagctccagaactaccgtaattatctgttgccagctgggtacagccttgaggagcaaagaattctggactggcaaccccgtgaaaatcctttccagaatctgaaggtactcttggtatcagaccaacagcagaacttcctggagctctggtctgagatcctcatgactggtggtgcagcctctgtgaagcagcaccattcaagtgcccataacaaagatattgctttaggggtatttgatgtggtggtgacggacccctcatgcccagcctcggtgctgaagtgtgctgaagcattgcagctgcctgtggtgtcacaagagtgggtgatccagtgcctcattgttggggagagaattggattcaagcagcatccaaaatataaacacgattatgtttctcactaa

SEQ ID NO: 42 - DNA, 3930 bp, Saccharomyces cerevisiae
atgtcaggccagttagttcaatggaaaagctctccagatcgagtcacccaaagcgctataaaggaagcactgcattctcccttggctgatggcgacatgaacgaaatgaatgttcccgttgatccgttggaaaacaaggtaaatagcacaaacataatcgaaggaagtcccaaagcaaatccaaatcctgtcaagtttatgaatacaagtgagatatttcaaaaatctctgggattacttgacgagagtccaagacatgatgatgagttaaatattgaagtaggagataatgatcgaccaaatgctaacatattgcataatgaaaggactcctgaccttgaccgaattgctaactttttcaaaagcaatcgaacccctggtaaagaaaatcttttgaccaaatatcaaagctccgatctggaagacactcctctgatgttaagaaaaaaaatgacttttcaaactccaactgatccattggaacagaaaaccttcaaaaagttgaagtcagatactgggttttgctattatggagagcagaatgatggagaagaaaatgcgtcattagaagttacagaggcggatgccacttttgtacagatggctgaacgttctgctgataattatgactgtgcattggaaggaattgttacacctaaaagatataaagacgaattaagtaaaagtggaggaatgcaagatgaacgagttcaaaaaactcaaatcatgatatcagcagaatcacctaattcgataagctcttatgacaagaacaaaattaccgggaatggccggaccacaagaaatgtaaacaaggtttttaacaataacgaagataacataggagctatcgaggaaaaaaatccagtaaaaaagaaaagtgagaactattcatcagatgatctcagagaacggaacaatcaaataatacaaagtaatgaatcagaggagattaacgaattggaaaagaatctgaatgtttcgggtagagagaatgacgtgaacaatttagatatcgatattaatagtgctgtgtctggcaccccttcacgcaacaatgcggaagaagaaatgtattccagtgagagtgtaaacaatcgggaaccatccaagaagtggatattccgatactcaaaagacaaaacggaaaataatagcaatagatctacgcaaatagtcaataatccaagaacacaggaaatgcctttagatagtatttcaatcgatacgcaacccttatctaaaagtttcaataccgaaacaaataatgaattagagacacagataattgtttcatcgctttcccaaggcatatctgctcagaagggacctgtttttcattctactggccagacagaagaaataaaaacccaaataataaattctcctgaacaaaatgctttgaatgcaacctttgaaactcccgttactctttctcggattaattttgaacccatattggaagttcctgagactagttcaccatctaagaatacgatgtcaaaaccctcgaattcttcacctattccgaaggaaaaagatacatttaatatacacgagagagaagtagagacaaacaatgttttttcaaacgatatacaaaattcttcaaatgcagctaccagagatgacattatcatagccggttcatctgatttcaacgaacaaaaggaaataaccgatagaatatacttacaactttcaggaaagcaaatatctgattcaggaagtgatgaaacagaacgtatgtccccaaatgagcttgatacgaaaaaggaaagtacaatcatgagcgaggttgaactaacccaagaactgcctgaagttgaagagcagcaagatcttcaaacgtctccaaaaaagctggtagtcgaggaagaaactttaatggagataaaaaaaagcaaggggaactcacttcagcttcatgatgataataaagaatgcaattcagataaacaagatggcacagagtctttggatgtagctttgattgaacacgaaagcaaaggacagagctcagaacttcagaaaaacctcatgcaattatttccaagtgagtcacaggagattattcagaaccgaagaacaataaagcgacgtcaaaaagatacaatagagatcggtgaagaggaggagaacagaagcactaagacatcaccgacaaaacacctcaaaagaaattcagatttggatgctgcttctatcaaaagggaaccgtcttgcagcattaccatacaaacaggggagacaggttcgggcaaagactctaaagaacagtcttacgtgtttcctgaaggtattagaacggcagataatagtttcttatcgaaagacgacataatttttggaaatgcggtatggtgtcagtatacgtggaattacaaattttatccgggtattttattggaagttgacactaatcaagatggctgttggatttatttcgaaacaggaagatcgctaaccaaagatgaggacatctactacttagatattagaataggggatgctgttacctttgatggaaatgagtacgtagtcgttggtctagaatgtcgtagccatgatctcaacataataagatgtattcgaggatatgatacggttcatttgaaaaaaaaaaatgcaagcggattgttggggaaaaggacgttaattaaagcactaagctcgatcagtcttgacctaagcgagtgggctaaaagagcgaagatcatattagaagataatgagaaaaataaaggcgacgcgtataggtacttgagacatcccattaggggaaggaaatcaatgaccaatgttctgtctccgaagaaacatactgatgacgaaaaggacataaatacgcatactgaagtgtacaataacgaaatagaatcgagctccgaaaagaaggaaattgttaaaaaggattctagagacgcattagctgaacatgcaggagcgccaagcctgcttttttcttctggtgaaatcagaacagggaatgtatttgataaatgtatttttgttttgacaagcctattcgaaaatagagaggaacttcgacagaccattgaatcgcaaggcggcactgtaattgagtcaggattttccactttatttaacttcactcatccgctagctaaatctttagtcaataaaggtaatacagataatattcgagaattggccttgaagctagcctggaaacctcattccctatttgcagactgcagatttgcttgcctaatcacaaaacggcatttaagaagcttaaagtacttagaaactttggcgttggggtggcctacactacactggaaattcataagtgcatgcattgaaaagaaaagaatagtaccacatttaatataccaatacctattaccttcgggtgaaagttttcggttatcgttagattctccatcaaagggaggaatcattaaatccaacaatattttttcattttatacacaattcttacgcggatctaatttaagagatcagatatgtggagtgaagaaaatgttaaatgactacattgttattgtttggggtagatctgagttggacagttttgtcaaatttgcttttgcatgtttgagcgcaggtagaatgcttacaattgatttacccaatattgatgtagatgatacagagccattgttaaatgccttagattctttagtacccagaataggatcagaattatctaatcgaaagttaaagtttctcatatatgctaacgaaaataatggtaaatctcagatgaagcttctcgaaagattgagaagtcaaatatcactgaaatttaagaaatttaattacatatttcacactgaatctaaagaatggctaattcagacaataattaacgaggacactggttttcacgatgatattacggacaatgatatatacaacactatttctgaggttagatga

SEQ ID NO: 43 - protein, 6 aa, Artificial Sequence
  Description of Artificial Sequence: Synthetic peptide
  MOD_RES (2)..(2): Any amino acid
  MOD_RES (5)..(5): Any amino acid
  MOD_RES (6)..(6): Ser or Thr
RXRSXX

SEQ ID NO: 44 - protein, 6 aa, Artificial Sequence
  Description of Artificial Sequence: Synthetic peptide
  MOD_RES (3)..(3): Any amino acid
  MOD_RES (4)..(4): Phosphorylated Ser or phosphorylated Thr
  MOD_RES (5)..(5): Any amino acid
RSXXXP

SEQ ID NO: 45 - protein, 4 aa, Artificial Sequence
  Description of Artificial Sequence: Synthetic peptide
  MOD_RES (1)..(1): Ser or Thr
  MOD_RES (3)..(3): Any amino acid
XPXR

SEQ ID NO: 46 - protein, 16 aa, Artificial Sequence
  Description of Artificial Sequence: Synthetic peptide
  MOD_RES (3)..(6): Any amino acid except Cys
  MISC_FEATURE (7)..(7): Phosphorylated
  MOD_RES (9)..(12): Any amino acid except Cys
MAXXXXTPXXXXAKKK

SEQ ID NO: 47 - protein, 15 aa, Artificial Sequence
  Description of Artificial Sequence: Synthetic peptide
  MOD_RES (3)..(6): Any amino acid except Cys
  MOD_RES (7)..(7): Phosphorylated Thr
  MOD_RES (8)..(11): Any amino acid except Cys
MAXXXXTXXXXAKKK

SEQ ID NO: 48 - protein, 16 aa, Artificial Sequence
  Description of Artificial Sequence: Synthetic peptide
  MOD_RES (3)..(6): Any amino acid except Cys
  MISC_FEATURE (8)..(8): Phosphorylated
  MOD_RES (9)..(12): Any amino acid except Cys
MAXXXXSTXXXXAKKK

SEQ ID NO: 49 - protein, 14 aa, Artificial Sequence
  Description of Artificial Sequence: Synthetic peptide
  MOD_RES (3)..(5): Any amino acid except Cys
  MISC_FEATURE (6)..(6): Phosphorylated
  MOD_RES (8)..(10): Any amino acid except Cys
MAXXXSPXXXAKKK

SEQ ID NO: 50 - protein, 15 aa, Artificial Sequence
  Description of Artificial Sequence: Synthetic peptide
  MOD_RES (3)..(6): Any amino acid except Cys
  MOD_RES (7)..(7): Phosphorylated Ser
  MOD_RES (8)..(11): Any amino acid except Cys
MAXXXXSXXXXAKKK

SEQ ID NO: 51 - protein, 6 aa, Artificial Sequence
  Description of Artificial Sequence: Synthetic peptide
  MISC_FEATURE (4)..(4): Phosphorylation
MQSTPL

SEQ ID NO: 52 - protein, 3 aa, Artificial Sequence
  Description of Artificial Sequence: Synthetic peptide
  MOD_RES (2)..(2): Phosphorylated Thr or phosphorylated Ser
  MOD_RES (3)..(3): Pro or any amino acid
SXX

SEQ ID NO: 53 - protein, 7 aa, Artificial Sequence
  Description of Artificial Sequence: Synthetic peptide
  MISC_FEATURE (5)..(5): Phosphorylation
LLCSTPN

SEQ ID NO: 54 - protein, 16 aa, Artificial Sequence
  Description of Artificial Sequence: Synthetic peptide
  MOD_RES (3)..(6): Any amino acid except Cys
  MISC_FEATURE (8)..(8): Phosphorylation
  MOD_RES (9)..(12): Any amino acid except Cys
MAXXXXSSXXXXAKKK

SEQ ID NO: 55 - protein, 6 aa, Artificial Sequence
  Description of Artificial Sequence: Synthetic peptide
  MOD_RES (2)..(2): Any amino acid
  MOD_RES (5)..(5): Any amino acid
  MOD_RES (6)..(6): Phosphorylated Ser or phosphorylated Thr
RXRSXX

SEQ ID NO: 56 - protein, 16 aa, Artificial Sequence
  Description of Artificial Sequence: Synthetic peptide
  MISC_FEATURE (8)..(8): Phosphorylation
MAGPMQSTPLNGAYKK

SEQ ID NO: 57 - protein, 16 aa, Artificial Sequence
  Description of Artificial Sequence: Synthetic peptide
  MOD_RES (3)..(5): Any amino acid except Cys
  MOD_RES (6)..(6): Ala, Ile, Leu, Met, Asn, Pro, Ser, Thr or Val
  MOD_RES (7)..(7): Phosphorylated Ser or phosphorylated Thr
  MOD_RES (9)..(9): A biased mixture of 25% E and 75% any amino acid except Arg, Cys, His or Lys
  MOD_RES (10)..(12): Any amino acid except Cys
MAXXXXXQXXXXAKKK

SEQ ID NO: 58 - protein, 16 aa, Artificial Sequence
  Description of Artificial Sequence: Synthetic peptide
  MOD_RES (3)..(6): Any amino acid except Cys
  MISC_FEATURE (7)..(7): Phosphorylation
  MOD_RES (8)..(12): Any amino acid except Cys
MAXXXXSXXXXXAKKK

SEQ ID NO: 59 - protein, 17 aa, Artificial Sequence
  Description of Artificial Sequence: Synthetic peptide
  MOD_RES (3)..(6): Any amino acid except Cys
  MISC_FEATURE (7)..(7): Phosphorylation
  MOD_RES (8)..(9): Any amino acid except Cys
  MOD_RES (11)..(12): Any amino acid except Cys
GAXXXXSXXFXXAYKKK

SEQ ID NO: 60 - protein, 9 aa, Artificial Sequence
  Description of Artificial Sequence: Synthetic peptide
  MOD_RES (4)..(4): Phosphorylated Ser or phosphorylated Thr
YDIXQVFPF

SEQ ID NO: 61 - protein, 20 aa, Artificial Sequence
  Description of Artificial Sequence: Synthetic peptide
  MOD_RES (1)..(1): Biotin-Aminohexanoic acid
  MOD_RES (3)..(3): Aminohexanoic acid
  MOD_RES (7)..(9): Any amino acid except Cys
  MOD_RES (10)..(10): A biased mixture of Ala, Ile, Leu, Met, Asn, Pro, Ser, Thr or Val
  MOD_RES (11)..(11): Phosphorylated Ser or phosphorylated Thr
  MOD_RES (13)..(13): A biased mixture of 25% E and 75% any amino acid except Arg, Cys, His or Lys
  MOD_RES (14)..(16): Any amino acid except Cys
XGXGGAXXXXXQXXXXAKKK

SEQ ID NO: 62 - protein, 16 aa, Artificial Sequence
  Description of Artificial Sequence: Synthetic peptide
  MOD_RES (3)..(5): Any amino acid except Cys
  MOD_RES (6)..(6): A biased mixture of amino acids Ala, Ile, Leu, Met, Asn, Pro, Ser, Thr, and Val
  MOD_RES (7)..(7): Phosphorylated Ser or phosphorylated Thr
  MOD_RES (9)..(9): A biased mixture of 25 % E, 75% any amino acid except Arg, Cys, His or Lys
  MOD_RES (10)..(12): Any amino acid except Cys
GAXXXXXQXXXXAKKK

SEQ ID NO: 63-64 - skipped (2 entries)

SEQ ID NO: 65 - protein, 16 aa, Artificial Sequence
  Description of Artificial Sequence: Synthetic peptide
  MISC_FEATURE (8)..(8): Phosphorylation
MAGPMQSSPLNGAYKK

SEQ ID NO: 66 - protein, 16 aa, Artificial Sequence
  Description of Artificial Sequence: Synthetic peptide
  MISC_FEATURE (8)..(8): Phosphorylation
MAGPMQSYPLNGAYKK

SEQ ID NO: 67 - protein, 15 aa, Artificial Sequence
  Description of Artificial Sequence: Synthetic peptide
  MISC_FEATURE (8)..(8): Phosphorylation
MAGPMQVTPLNGAKK

SEQ ID NO: 68 - protein, 16 aa, Artificial Sequence
  Description of Artificial Sequence: Synthetic peptide
  MISC_FEATURE (8)..(8): Phosphorylation
MAGPMQATPLNGAYKK

SEQ ID NO: 69 - protein, 16 aa, Artificial Sequence
  Description of Artificial Sequence: Synthetic peptide
  MISC_FEATURE (8)..(8): Phosphorylation
MAGPMQGTPLNGAYKK

SEQ ID NO: 70 - protein, 16 aa, Artificial Sequence
  Description of Artificial Sequence: Synthetic peptide
  MISC_FEATURE (8)..(8): Phosphorylation
MAGPMQCTPLNGAYKK

SEQ ID NO: 71 - protein, 16 aa, Artificial Sequence
  Description of Artificial Sequence: Synthetic peptide
  MISC_FEATURE (8)..(8): Phosphorylation
MAGPMQTTPLNGAYKK

SEQ ID NO: 72 - protein, 15 aa, Artificial Sequence
  Description of Artificial Sequence: Synthetic peptide
  MISC_FEATURE (8)..(8): Phosphorylation
MAGPMQSTNLNGAKK

SEQ ID NO: 73 - protein, 13 aa, Artificial Sequence
  Description of Artificial Sequence: Synthetic peptide
AQLLCSTPNGLDR

SEQ ID NO: 74 - protein, 13 aa, Artificial Sequence
  Description of Artificial Sequence: Synthetic peptide
PRLLCSTPSFKKT

SEQ ID NO: 75 - protein, 603 aa, Rattus norvegicus
MNAAAKAGKLARAPADLGKGGVPGDAVPGAPVAAPLAKEIPEVLVDPRSRQQYVRGRFLGKGGFAKCFEISDSDTKEVFPGKIVPKSLLLKPHQKEKMSMETSIHRSLEHQHVVGFHGFFEDSDFVFVVLELCRRRSLLELHKRRKALTEPEARYYLRQIVLGCQYLHRNQVIHRDLKLGNLFLNEDLEVKIGDFGLATKVEYEGERKKTLCGTPNYIAPEVLSKKGHSFEVDVWSIGCIMYTLLVGKPPFETSCLKETYLRIKKNEYSIPKHINPVAASLIQKMLQTDPAARPTIHELLNDEFFTSGYIPARLPITCLTIPPRFSIAPSSLDPSNRKPLTVLNKGVENPLPDRPREKEEPVVRETNEAIECHLSDLLQQLTSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLNYFRNYMSEHLLKAGANITPREGDELARLPYLRTWFRTRSAIILHLSNGTVQINFFQDHTKLIRGPLMAAVTYINEKRDFRTYRLSLLEEYGCCKELASRLRYARTMVDKLLSSRSACNRLKAS

SEQ ID NO: 76 - protein, 648 aa, Caenorhabditis elegans
MNRLPNIAKPPQKSNQRKEKAPPEVPALIADKDRGTYYEKGRFLGKGGFAHCYELTNRATREVVAGKVVPKSMLVKQYQRDKMTQEVQIHRELGHINIVKLFNFFEDNLNVYITLELCARRSLMELHKRRKAVTEPEARYFTHQIVDGVLYLHDLNIIHRDMKLGNLFLNDDLVVKIGDFGLATTVNGDERKKTLCGTPNYIAPEVLNKAGHSFEVDIWAVGCILYILLFGQPPFESKSLEETYSRIRHNNYTIPSIATQPAASLIRKMLDPEPTRRPTAKQVQRDGFFKSGFMPTRLPVSCLTMVPKFGGHETSMMEENVAPRGVDARSQRPLNGRAGLSALPQHIVSNNADRERAQQQAAEATFREPEDAYLSQLFHQVAVLLEQRIPGLEEEEAALDGYQSPECLPVFWISKWVDYSDKYGIGYQLCDNSVGVLFNDNSRIMLDQAGNELTYIEKSNKEHYFSMHSGEMPGLLNKKVTLLKYFRSYMNDHLVKAGEGSEQRAGDDLARLPTLRVWFRTKSAIVLHLSNGTVQINFFNDHVKMMMCPLMQAVTFIDQNKRMLTYKLNNLQRNGCPEKFLHRLKYAKTMIERLMSDANVVSQNPARQPDMPRSMAAARSASAGSRGPNQAASHLPQSASGSNIHPRR

SEQ ID NO: 77 - protein, 278 aa, Homo sapiens
SIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 78 - protein, 282 aa, Xenopus laevis
SIAPSTIDQSLRKPLTAINKGQDSPLVEKQVAPAKEEEMQQPEFTEPADCYLSEMLQQLTCLNAVKPSERALIRQEEAEDPASIPIFWISKWVDYSDKYGLGYQLCDNSVGVLFNDSTRLIMYNDGDSLQYIERNNTESYLNVRSYPTTLTKKITLLKYFRNYMSEHLLKAGANTTPREGDELARLPFLRTWFRTRSAIILHLSNGTVQINFFQDHTKIILCPLMAAVSYIDEKREFRTYKLSLIQEFGCCKELASRLRYARTMVEKLQSSKSAVAHVKASA

SEQ ID NO: 79 - protein, 279 aa, Drosophila melanogaster
GSNDTIEDSMHRKPLMEMNGIRPDDTRLESTFLKANLHDAITASAQVCRHSEDYRSDIESLYQQLTNLINGKPRILQGNLGDENTDPAAQPLFWISKWVDYSDKYGFGYQLCDEGIGVMFNDTTKLILLPNQINVHFIDKDGKETYMTTTDYCKSLDKKMKLLSYFKRYMIEHLVKAGANNVNIESDQISRMPHLHSWFRTTCAVVMHLTNGSVQLNFSDHMKLILCPRMSAITYMDQEKNFRTYRFSTIVENGVSKDLYQKIRYAQEKLRKMLEKMFT

SEQ ID NO: 80 - protein, 13 aa, Homo sapiens
AQLLCSTPNGLDR

SEQ ID NO: 81 - protein, 13 aa, Xenopus laevis
PRLLCSTPSFKKT

SEQ ID NO: 82 - protein, 197 aa, Homo sapiens
PIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHIKLILCPLMAAVTYIDEKRDFRTYRLSLLEEYGCCKELASRLRYARTMVDKLLSSRSASNRLKAS

SEQ ID NO: 83 - protein, 197 aa, Mus musculus
PIFWVSKWVDYSDKYGLGYQLCDNSVGVLFNDSTRLILYNDGDSLQYIERDGTESYLTVSSHPNSLMKKITLLNYFRNYMSEHLLKAGANITPREGDELARLPYLRTWFRTRSAIILHLSNGTVQINFFQDHTKLILCPLMAAVTYINEKRDFQTYRLSLLEEYGCCKELASRLRYARTMVDKLLSSRSASNRLKAS

SEQ ID NO: 84 - protein, 197 aa, Rattus norvegicus
PIFWVSKWVDYSDKYGLGYDLCDNSVGVLFNDSTRLILYNDGDSLQYIERDGTESYLTVSSHPNSLMKKITLLNYFRNYMSEHLLKAGANITPREGDELARLPYLRTWFRTRSAIILHLSNGTVQINFFQDHTKLIRGPLMAAVTYINEKRDFRTYRLSLLEEYGCCKELASRLRYARTMVDKLLSSRSACNRLKAS

SEQ ID NO: 85 - protein, 210 aa, Caenorhabditis elegans
PVFWISKWVDYSDKYGIGYQLCDNSVGVLFNDNSRIMLDQAGNELTYIEKSNKEHYFSMHSGEMPGLLMKKNTLLKYFRSYMNDHLVKAGEGSEQRAGDDLARLPTLRVWFRTKSAIVLHLSNGTVQINFFNDHVKMMMCPLMQAVTFIDQNKRMLTYKLNNLQRNGCPEKFLHRLKYAKTMIERLMDSANVVSQNPARQPDMPRSMAAA

SEQ ID NO: 86 - protein, 189 aa, Drosophila melanogaster
PLFWISKWVDYSDKYGFGYQLCDEGIGVMFNDTTKLILLPNQINVHFIDKDGKETYMTTTDYCKSLDKKMKLLSYFKRYMIEHLVKAGANNVNIESDQISRMPHLHSWFRTTCAVVMHLTNGSVQLNFSDHMKLILCPRMSAITYMDQEKNFRTYRFSTIVENGVSKDLYQKIRYAQEKLRKMLEKMFT

SEQ ID NO: 87 - protein, 198 aa, Xenopus laevis
PIFWISKWVDYSDKYGLGYQLCDNSVGVLFNDSTRLIMYNDGDSLQYIERNNTESYLNVRSYPTTLTKKITLLKYFRNYMSEHLLKAGANTTPREGDELARLPFLRTWFRTRSAIILHLSNGTVQINFFQDHTKIILCPLMAAVSYIDEKREFRTYKLSLIQEFGCCKELASRLRYARTMVEKLQSSKSAVAHVKASA

SEQ ID NO: 88 - protein, 195 aa, Helicobacter pylori
PILWVSKWVDYSDKYGLGYQLCDGSVGVLFNDSTRLLLHANADTLEYIERDGNEKYCRLGSYDSTLHKKVTLLKYFRNYMSEHLLKAGAAMTPRESDSMARLPFLQSWFRTKSAIVLHLSNGTVQINFFEDHTKLIVCPMMGAATYIDAKRNFRTFRLNLIEKHGCTPDLYDRIKYANNMVKNMLDKKTTTAAAH

SEQ ID NO: 89 - protein, 186 aa, Homo sapiens
SFQWVTKWVDYSNKYGFGYQLSDHTVGVLFNNGAHMSLLPDKKTVHYYAELGQCSVFPATDAREQFISQVTVLKYFSHYMEENLMDGGDLPSVTDIRRPRLYLLQWLKSDKALMMLFNDGTFQVNFYHDHTKIIICSQNEEYLLTYINEDRISTTFRLTTLLMSGCSSELKNRMEYALNMLLQRCN

SEQ ID NO: 90 - protein, 186 aa, Mus musculus
SFQWVTKWVDYSNKYGFGYQLSDHTVGVLFNNGAHMSLLPDKKTVHYYAELGQCSVFPATDAPEQFISQVTVLKYFSHYMEENLMDGGDLPSVTDIRRPRLYLLQWLKSDKALMMLFNDGTFQVNFYHDHTKIIICNQSEEYLLTYINEDRISTTFRLTTLLMSGCSLELKNRMEYALNMLLQRCN

SEQ ID NO: 91 - protein, 186 aa, Rattus norvegicus
SFQWVTKWVDYSNKYGFGYQLSDHTVGVLFNNGAHMSLLPDKKTVHYYAELGQCSVFPATDAPEQFISQVTVLKYFSHYMEENLMDGGDLPSVTDIRRPRLYLLQWLKSDKALMMLFNDGTFQVNFYHDHTKIIICNQNEEYLLTYINEDRISTTFRLTTLLMSGCSLELKHRMEYALNMLLQRCN

SEQ ID NO: 92 - protein, 214 aa, Caenorhabditis elegans
PIFWVSQWVHFPNHGIGYRLCENSSGMLFNDNTQMKVNSAGNQLTFVDENNTEQFYTMNDQVPMNLQTKINIFSNVQSYMNTHLEADTHLEAEDQCVNKVPPLRNFARLPTIQNWFETKSAVIFHLSNGTVQINFLDHVKMVLCPLLKSVTFIEENKRVSTFTFANILTNSCPKKYLSRIEYAQAKIKLLRPTNNQEHVVYVDSPCRPTTSNTA

SEQ ID NO: 93 - protein, 187 aa, Xenopus laevis
SFHWVTKWVDYSNKYGFGYQLSDHTVGVLFNNGAHMSFLPDKKTVHYYAELGQCSVFPATEAPEQFISQVTVLKYFSHYMEENLMDGGDLPSVTDVCRPRLYLLQWLKSDKALMMLFNDGTFQVNFYHDHTKIIIANQNDEYVLTYINEDRMSTTFHLSTLLISGCSPDLRQRLRYALRLLRDRSPA

SEQ ID NO: 94 - protein, 187 aa, Homo sapiens
PLVWFSEWVGFSNKFGFGYQLSSRRVAVLFNDGTHMALSANRKTVHYNPTSTKHFSFSVGAVRRALQPQLGILRYFASYMEQHLMKGGDLPSVEEVEVPAPPLLLQWVKTDQALLMLFSDGTVQVNFYGDHTKLILSGWEPLLVTFVARNRSACTYLASHLRQLGCSPDLRQRLRYALRLLRDRSPA

SEQ ID NO: 95 - protein, 187 aa, Mus musculus
PLVWVSKWVDYSNKFGFGYQLSSRRVAVLFNDGTHMALSANRKTVHYNPTSTKHFSFSMGSVPRALQPQLGILRYFASYMEQHLMKGGDLPSVEEAEVPAPPLLLQWVKTDQALLMLFSDGTVQVNFYGDHTKLILSGWEPLLVTFVARNRSACTYLASHLRQLGCSPDLRQRLRYALRLLRDQSPA

SEQ ID NO: 96 - protein, 186 aa, Rattus norvegicus
PLVWVSKWVDYSNKFGFGYQLSSRRVAVLFNDGTHMALSANRKTVHYNPTSTKHFSFSVGSVPRALRPQLGILRYFASYMEQHLMKGGDLPSVEEVEVPAPPLLLQWVKTDQALLMLFSDGTVQVNFYGDHTKLILSGWEPLLVTFVARNRSACTYLASHLRQLGCSPDLRQRLRYALRLLRDQSP

SEQ ID NO: 97 - protein, 186 aa, Xenopus laevis
HFVWVSKWVDYSNKYGFGYQLSNRSIGVLFNSGTHMVFPAHRRHVHYNLTNSRHFVPTSTVPEQLQGQMSILQYFATYMEKNLMKGGDLPCHEEGSQAPLLLLQWVKTEHALLMLFSNGTLQVNFYNDHTKIILCKPQDAYLLTYINRDRNSQTFLLSTLAQTGCNSEMYHRLKYTVKLLQQKAES

SEQ ID NO: 98 - protein, 194 aa, Schizosaccharomyces pombe
PVLFITKWVDYSNKYGLGYQLSDESVGVHFNDDTSLLFSADEEVVEYALHPKDTEIKPYIYPASKVPESIRSKLQLLKHFKSYMGQNLSKAVQDESFEKPKNSTSNTMLFMQHYLRTRQAIMFRLSNGIFQFNELDHRKVVISSTARKIIVLDKERERVELPLQEASAFSEDLRSRLKYIRETLESWASKMEVS

SEQ ID NO: 99 - protein, 196 aa, Saccharomyces cerevisiae
HPMIVTKWVDYSNKHGFSYQLSTEDIGVLFNNGTTVLRLADAEEFWYISYDDREGWVASHYLLSEKPRELSRHLEVVDFFAKYMKANLSRVSTFGREEYHKDDVFLRRYTRYKPFVMFELSDGTFQFNFKDHHKMAISDGGKLVTYISPSHESTTYPLVEVLKYGEIPGYPESNFREKLTLIKEGLKQKSTIVTVD

SEQ ID NO: 100 - protein, 99 aa, Homo sapiens
PIFWVSKWVDYSDKYGLGYQLCDNSVGVLFNDSTRLILYNDGDSLQYIERDGTESYLTVSSHPNSLMKKITLLKYFRNYMSEHLLKAGANITPREGDEL

SEQ ID NO: 101 - protein, 98 aa, Homo sapiens
ARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 102 - protein, 87 aa, Mus musculus
SAQLLKSVFVKNVGWATQLTSGAVWVQFNDGSQLVVQAGVSSISYTSPDGQTTRYGENEKLPEYIKQKLQCLSSILLMFSNPTPNFQ

SEQ ID NO: 103 - protein, 5 aa, Artificial Sequence
  Description of Artificial Sequence: Synthetic peptide
  MOD_RES (1)..(1): Met, Tyr, His, Phe, Lys, Ile or Leu
  MOD_RES (2)..(2): Ala, His, Met, Thr, Phe or Gln
  MOD_RES (3)..(3): Ser, Ala or Gly
  MOD_RES (4)..(4): Phosphorylated Ser or phosphorylated Thr
  MOD_RES (5)..(5): Pro, Met or any amino acid except Cys
XXXXX

SEQ ID NO: 104 - protein, 8 aa, Artificial Sequence
  Description of Artificial Sequence: Synthetic peptide
  MOD_RES (1)..(1): Pro or Phe
  MOD_RES (2)..(2): Met, Phe, Pro, Leu or Ile
  MOD_RES (3)..(3): Met, Tyr, His, Phe, Lys or Leu
  MOD_RES (4)..(4): Ala, His, Met, Thr, Phe or Gln
  MOD_RES (5)..(5): Ser or Ala
  MISC_FEATURE (6)..(6): Phosphorylation
  MOD_RES (7)..(7): Pro or Met
  MOD_RES (8)..(8): Phe, Ile, Lys, Leu or Val
XXXXXTXX

SEQ ID NO: 105 - protein, 8 aa, Artificial Sequence
  Description of Artificial Sequence: Synthetic peptide
  MOD_RES (1)..(1): Pro, Phe or Ile
  MOD_RES (2)..(2): Phe, Ile, Met, Leu, Pro or Val
  MOD_RES (3)..(3): Gln, Met, His, Phe, Ile or Thr
  MOD_RES (4)..(4): Thr, His or Gln
  MISC_FEATURE (6)..(6): Phosphorylation
  MOD_RES (8)..(8): Phe, Leu, Ile, Val or Lys
XXXXSTPX

SEQ ID NO: 106 - protein, 8 aa, Artificial Sequence
  Description of Artificial Sequence: Synthetic peptide
  MOD_RES (2)..(2): Ile, Leu, Val, Phe or Pro
  MOD_RES (3)..(3): Met, Leu, Phe, Ala or Ile
  MOD_RES (4)..(4): Thr or His
  MISC_FEATURE (6)..(6): Phosphorylation
  MOD_RES (7)..(7): Pro, Asp or Glu
  MOD_RES (8)..(8): Lys, Val or Phe
PXXXSTXX

SEQ ID NO: 107 - protein, 8 aa, Artificial Sequence
  Description of Artificial Sequence: Synthetic peptide
  MOD_RES (1)..(1): Pro or Phe
  MOD_RES (2)..(2): Phe, Ile, Leu, Met or Pro
  MOD_RES (3)..(3): Phe, Leu or Met
  MOD_RES (4)..(4): Thr, His or Gln
  MISC_FEATURE (6)..(6): Phosphorylation
  MOD_RES (8)..(8): Lys, Ile, Leu or Val
XXXXSTPX

SEQ ID NO: 108 - protein, 8 aa, Artificial Sequence
  Description of Artificial Sequence: Synthetic peptide
  MOD_RES (1)..(1): Pro or Phe
  MOD_RES (2)..(2): Phe, Ile, Leu, Met or Val
  MOD_RES (3)..(3): Ala, Met, Phe, Val or Ile
  MOD_RES (4)..(4): Thr, Ala, Gln, Met or His
  MISC_FEATURE (6)..(6): Phosphorylation
  MOD_RES (8)..(8): Leu, Ile or any amino acid other than Cys
XXXXSTPX

SEQ ID NO: 109 - protein, 222 aa, Homo sapiens
ALSDMLQQLHSVNASKPSERGLVRQAEAEDPACIPIFWVSKWVDYSDKYGLGYQLCDNSVGVLFNNSTRLILYNDGDSLQYIERDGTESYLTVSSHPNALMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSA

SEQ ID NO: 110 - protein, 10 aa, Artificial Sequence
  Description of Artificial Sequence: Synthetic peptide
  MOD_RES (1)..(1): Any amino acid except Cys
  MOD_RES (3)..(3): Gly, Asp or Glu
  MOD_RES (4)..(4): Leu, Ile, Met or Val
  MOD_RES (5)..(5): Phosphorylated Ser or phosphorylated Tyr
  MOD_RES (7)..(7): Val or Ile
  MOD_RES (8)..(8): Phe, Leu or Ile
  MOD_RES (10)..(10): Ile, Phe, Leu, Val or Tyr
XYXXXQXXPX

SEQ ID NO: 111 - protein, 10 aa, Artificial Sequence
  Description of Artificial Sequence: Synthetic peptide
  MOD_RES (1)..(2): Any amino acid except Cys
  MOD_RES (4)..(4): Ile, Met, Val or Leu
  MISC_FEATURE (5)..(5): Phosphorylation
  MOD_RES (6)..(6): Phe, Ile, Gln or Tyr
  MOD_RES (7)..(7): Val or Thr
  MOD_RES (9)..(9): Any amino acid except Cys
  MOD_RES (10)..(10): Ile, Phe, Met or Leu
XXEXSXXFXX

SEQ ID NO: 112 - protein, 10 aa, Artificial Sequence
  Description of Artificial Sequence: Synthetic peptide
  MOD_RES (3)..(3): Asp or Glu
  MOD_RES (4)..(4): Leu, Ile or Met
  MISC_FEATURE (5)..(5): Phosphorylation
  MOD_RES (6)..(6): Gln, Ile or Pro
  MOD_RES (7)..(7): Val or Ile
  MOD_RES (8)..(8): Phe, Ile, Val, Leu or Tyr
GYXXSXXXFY

SEQ ID NO: 113 - protein, 10 aa, Artificial Sequence
  Description of Artificial Sequence: Synthetic peptide
  MOD_RES (1)..(3): Any amino acid except Cys
  MOD_RES (4)..(4): Ile, Leu or Trp
  MISC_FEATURE (5)..(5): Phosphorylation
  MOD_RES (6)..(6): Gln, Phe or Ile
  MOD_RES (8)..(8): Ile, Leu or Val
  MOD_RES (9)..(9): Phe, Tyr or Pro
XXXXTXYXXA

SEQ ID NO: 114 - protein, 10 aa, Artificial Sequence
  Description of Artificial Sequence: Synthetic peptide
  MOD_RES (1)..(1): Any amino acid except Cys
  MOD_RES (2)..(2): Phe or Tyr
  MOD_RES (3)..(3): Asp or Glu
  MOD_RES (4)..(4): Ile, Val, Leu or Met
  MOD_RES (5)..(5): Phosphorylated Ser or phosphorylated Thr
  MOD_RES (7)..(7): Val, Thr, Ile or Ser
  MOD_RES (8)..(8): Phe or Tyr
  MOD_RES (9)..(9): Val or Pro
  MOD_RES (10)..(10): Phe or Gly
XXXXXQXXXX

SEQ ID NO: 115 - protein, 10 aa, Artificial Sequence
  Description of Artificial Sequence: Synthetic peptide
  MOD_RES (1)..(1): Any amino acid except Cys
  MOD_RES (2)..(2): Arg or Tyr
  MOD_RES (3)..(3): Glu or Asp
  MOD_RES (4)..(4): Val, Ile or Met
  MISC_FEATURE (5)..(5): Phosphorylation
  MOD_RES (6)..(6): Phe, Tyr, Ile or Gln
  MOD_RES (7)..(7): Thr or Val
  MOD_RES (9)..(9): Any amino acid except Cys
  MOD_RES (10)..(10): Phe, Met or Tyr
XXXXSXXFXX

SEQ ID NO: 116 - protein, 10 aa, Artificial Sequence
  Description of Artificial Sequence: Synthetic peptide
  MOD_RES (1)..(2): Any amino acid except Cys
  MOD_RES (4)..(4): Any amino acid except Cys
  MISC_FEATURE (5)..(5): Phosphorylation
  MOD_RES (6)..(6): Gln or Phe
  MOD_RES (7)..(7): Val or Ile
  MOD_RES (8)..(8): Phe or Tyr
  MOD_RES (10)..(10): Any amino acid except Cys
XXYXSXXXIX

SEQ ID NO: 117 - protein, 10 aa, Artificial Sequence
  Description of Artificial Sequence: Synthetic peptide
  MOD_RES (1)..(1): Any amino acid except Cys
  MOD_RES (3)..(3): Asp or Glu
  MOD_RES (4)..(4): Ile or Leu
  MISC_FEATURE (5)..(5): Phosphorylation
  MOD_RES (6)..(6): Gln, Glu or Phe
  MOD_RES (7)..(7): Asp, Tyr or Ile
  MOD_RES (8)..(8): Phe or Tyr
  MOD_RES (9)..(9): Asp or Pro
XEXXTXXXXA

SEQ ID NO: 118 - protein, 10 aa, Artificial Sequence
  Description of Artificial Sequence: Synthetic peptide
  MOD_RES (1)..(3): Any amino acid except Cys
  MOD_RES (4)..(4): Ala, Ile, Leu, Met, Asn, Pro, Ser, Thr or Val
  MOD_RES (5)..(5): Phosphorylated Ser or phosphorylated Thr
  MOD_RES (7)..(7): A biased mixture of 25% E, 75% of any amino acid except Arg, Cys, Lys or His
  MOD_RES (8)..(10): Any amino acid except Cys
XXXXXQXXXX

SEQ ID NO: 119 - protein, 10 aa, Artificial Sequence
  Description of Artificial Sequence: Synthetic peptide
  MOD_RES (1)..(1): Any amino acid
  MOD_RES (2)..(2): Cys, Phe, Trp or Tyr
  MOD_RES (3)..(3): Ile, Leu or Val
  MOD_RES (4)..(4): Any amino acid with aliphatic or aromatic side chains
  MISC_FEATURE (5)..(5): Phosphorylation
  MOD_RES (7)..(7): Asp, Glu, Met or Asn
  MOD_RES (8)..(8): Phe or Ile
  MOD_RES (9)..(9): Gly, His, Lys or Pro
  MOD_RES (10)..(10): Phe, Lys, Trp or Tyr
XXXXTQXXXX

SEQ ID NO: 120 - protein, 10 aa, Artificial Sequence
  Description of Artificial Sequence: Synthetic peptide
  MOD_RES (1)..(1): Any amino acid
  MOD_RES (2)..(2): Phe, Trp or Tyr
  MOD_RES (3)..(3): Ile, Leu or Val
  MOD_RES (4)..(4): Any amino acid with an aliphatic or aromatic side chain
  MISC_FEATURE (5)..(5): Phosphorylation
  MOD_RES (7)..(7): Ala, Met, Asn, Pro or Gln
  MOD_RES (8)..(8): Cys or Phe
  MOD_RES (9)..(9): Asp, Glu, Gly, Lys or Pro
  MOD_RES (10)..(10): Phe, Gly, His or Lys
XXXXTQXXXX

SEQ ID NO: 121 - protein, 10 aa, Artificial Sequence
  Description of Artificial Sequence: Synthetic peptide
  MOD_RES (1)..(4): Any amino acid
  MISC_FEATURE (5)..(5): Phosphorylation
  MOD_RES (6)..(7): Any amino acid
  MOD_RES (8)..(8): Phe, Ile, Leu, Asn or Tyr
  MOD_RES (9)..(10): Any amino acid
XXXXSXXXXX

SEQ ID NO: 122 - protein, 10 aa, Artificial Sequence
  Description of Artificial Sequence: Synthetic peptide
  MOD_RES (1)..(4): Any amino acid
  MISC_FEATURE (5)..(5): Phosphorylation
  MOD_RES (6)..(7): Any amino acid
  MOD_RES (8)..(8): Phe or Tyr
  MOD_RES (9)..(9): Phe, Ile, Leu, Val, Trp or Tyr
  MOD_RES (10)..(10): Any amino acid
XXXXSXXXXX

SEQ ID NO: 123 - protein, 16 aa, Artificial Sequence
  Description of Artificial Sequence: Synthetic peptide
  MISC_FEATURE (7)..(7): Phosphorylation
GAAYDISQVFPFAKKK

SEQ ID NO: 124 - protein, 16 aa, Artificial Sequence
  Description of Artificial Sequence: Synthetic peptide
  MISC_FEATURE (7)..(7): Phosphorylation
GAAYDITQVFPFAKKK

SEQ ID NO: 125 - protein, 16 aa, Artificial Sequence
  Description of Artificial Sequence: Synthetic peptide
GAAYDISQVFPFAKKK

SEQ ID NO: 126 - protein, 16 aa, Artificial Sequence
  Description of Artificial Sequence: Synthetic peptide
GAAYDITQVFPFAKKK

SEQ ID NO: 127 - protein, 16 aa, Artificial Sequence
  Description of Artificial Sequence: Synthetic peptide
  MISC_FEATURE (7)..(7): Phosphorylation
GAAYDISQVFPFAKKK

SEQ ID NO: 128 - protein, 16 aa, Artificial Sequence
  Description of Artificial Sequence: Synthetic peptide
GAAYDISQVFPFAKKK

SEQ ID NO: 129 - protein, 16 aa, Artificial Sequence
  Description of Artificial Sequence: Synthetic peptide
GAAYDITQVFPFAKKK